(19) United States
(12) Patent Application Publication
(10) Pub. No.: US 2020/0362054 A1
(43) Pub. Date: Nov. 19, 2020

(54) TRISPECIFIC BINDING MOLECULES AGAINST TUMOR-ASSOCIATED ANTIGENTS AND USE THEREOF

(71) Applicants: Brian GRANDA, Salisbury, MA (US); Connie HONG, Somerville, MA (US); Melissa RAMONES, West Roxbury, MA (US); Darko SKEGRO, Grenchen (CH)

(72) Inventors: Brian GRANDA, Salisbury, MA (US); Connie HONG, Somerville, MA (US); Melissa RAMONES, West Roxbury, MA (US); Darko SKEGRO, Grenchen (CH)

(21) Appl. No.: 16/765,830

(22) PCT Filed: Nov. 20, 2018
(86) PCT No.: PCT/US2018/062078

§ 371 Date: May 20, 2020

Related U.S. Application Data
(60) Provisional application No. 62/589,331, filed on Nov. 21, 2017.

(51) Int. Cl. C07K 16/30; C07K 16/28; A61P 35/00

(52) U.S. Cl. CPC C07K 16/30; C07K 16/2806; C07K 16/2809; C07K 2317/31; C07K 2317/55; C07K 2317/569; C07K 2317/64; A61P 35/00

(57) ABSTRACT

The present disclosure provides trispecific binding molecules that specifically bind to CD2, CD3 and a tumor-associated antigen, conjugates comprising the trispecific binding molecules, and pharmaceutical compositions comprising the trispecific binding molecules and the conjugates. The disclosure further provides methods of using the trispecific binding molecules to treat cancers that express the tumor-associated antigens. The disclosure yet further provides recombinant host cells engineered to express the trispecific binding molecules and methods of producing the trispecific binding molecules by culturing the host cells under conditions in which the trispecific binding molecules are expressed.

### 1. CROSS-REFERENCE TO RELATED APPLICATIONS

This application claims the priority benefit of U.S. provisional application No. 62/589,331, filed Nov. 21, 2017, the contents of which are incorporated herein in their entireties by reference thereto.

### 2. SEQUENCE LISTING

The instant application contains a Sequence Listing which has been submitted electronically in ASCII format and is hereby incorporated by reference in its entirety. Said ASCII copy, created on Nov. 19, 2018, is named NOV-001WO_SL.txt and is 592,187 bytes in size.

### 3. BACKGROUND

Redirected targeted T-cell lysis (RTCC) is an exciting mechanism for first line treatment and refractory settings. Antibodies and antibody fragments with their exquisite selectivity have been successfully engineered in a variety of formats to allow for the dual specificities required tso cross-link T-cells to a single receptor on the target cell.

First line treatments for some indications or the indications themselves may promote immune suppressive environments to promote T-cell anergy, reducing the efficacy of existing RTCC therapies. Acute myeloid lymphoma, for example, has been shown to be a particularly evasive disease in the context of avoid immune surveillance (Teague and Klein, 2013, Journal for ImmunoTherapy of Cancer 1:13). The solid tumor micro-environment can also provide mechanisms of escape and promote survival of tumor cells through a variety of pathways (Vaney et al., 2015, Seminars in Cancer Biology 35:S151-S184).

Therefore, there is a need for improved RTCC approaches.

### 4. SUMMARY

The present disclosure extends the principles of RTCC by providing mutispecific binding molecules that engage a tumor-associated antigen (“TAA”) and CD2 in addition to CD3 or other component of a TCR complex on T-cells.

The present invention is based, at least in part, on the finding that engaging CD2 in addition to a component of a TCR complex will improve the clinical outcomes of RTCC therapy by activating T cell subpopulations that would be refractory to stimulation using bispecific engagers that target only a TAA and a TCR complex. As shown in the Examples, the outcome of treating tumors with trispecific binding molecules that engage a TAA, CD2 and CD3 results in improved anti-tumor activity as compared to engaging the TAA and CD3 only, particularly in the presence of anergic T-cells. Without being bound by theory, the inventors believe that combining CD2- and TCR complex-engagement in a single multispecific molecule can stimulate both a primary signaling pathway that promotes T-cell mediated lysis of tumor cells (by clustering TCRs, for example) and a second co-stimulatory pathway to induce T-cell proliferation and potentially overcome anergy.

Accordingly, the present disclosure provides trispecific binding molecules (“TBMs”) that bind to (1) a tumor-associated antigen (“TAA”), (2) CD2, and (3) CD3 or other component of a TCR complex. The TBMs comprise at least three antigen-binding modules (“ABMs”) that can bind TAA, CD2 and a component of a TCR complex. In some embodiments, each antigen-binding module is capable of binding its respective target at the same time as each of the other antigen-binding modules is bound to its respective target. Each ABM may be immunoglobulin- or non-immunoglobulin-based, and therefore the TBMs of the disclosure can include immunoglobulin-based ABMs, non-immunoglobulin-based ABMs, or a combination thereof. Immunoglobulin-based ABMs that can be used in the TBMs of the disclosure are described in Section 6.2.1 and specific embodiments 31 to 406, 575 to 583, 591 to 660, 662 to 667, 671, 673 to 762 and 830 to 898, infra. Non-immunoglobulin-based ABMs that can be used in the TBMs of the disclosure are described in Section 6.2.2 and specific embodiments 2 to 30, 403 to 406 and 584 to 589, infra. Further features of exemplary ABMs that bind to a component of a TCR complex are described in Section 6.5 and specific embodiments 38 to 106, 590 to 667, and 1045, infra. Further features of exemplary ABMs that bind to CD2 are described in Section 6.6 and specific embodiments 2 to 37, 403 to 406, 575 to 589, and 1044, infra. Further features of exemplary ABMs that bind to TAAs are described in Section 6.7 and specific embodiments 107 to 276, 346 to 406, 668 to 762, and 1046 infra.

The ABMs of a TBM of the disclosure (or portions thereof) can be connected to each other, for example, by short peptide linkers or by an Fc domain. Methods and components for connecting ABMs to form a TBM are described in Section 6.3 and specific embodiments 401 to 453, 763 to 829 and 899 to 957, infra.

TBMs of the disclosure have at least three ABMs (i.e., a TBM is at least trivalent), but can also have more than three ABMs. For example, a TBM can have four ABMs (i.e., is tetravalent), five ABMs (i.e., is pentavalent), or six ABMs (i.e., is hexavalent), provided that the TBM has at least one ABM that can bind a TAA, at least one ABM that can bind CD2, and at least one ABM that can bind a component of a TCR complex. Exemplary trivalent, tetravalent, pentavalent, and hexavalent TBM configurations are shown in FIG. 1 and described in Section 6.4 and specific embodiments 958 to 1042, infra.

The disclosure further provides nucleic acids encoding the TBMs of the disclosure (either in a single nucleic acid or a plurality of nucleic acids) and recombinant host cells and cell lines engineered to express the nucleic acids and TBMs of the disclosure. Exemplary nucleic acids, host cells, and cell lines are described in Section 6.8 and specific embodiments 570 to 573 and 1169 to 1176, infra.

The present disclosure further provides drug conjugates comprising the TBMs of the disclosure. Such conjugates are referred to herein as “antibody-drug conjugates” or “ADCs” for convenience, notwithstanding that some or all of the ABMs can be non-immunoglobulin domains. Examples of ADCs are described in Section 6.9 and specific embodiments 469 to 507 and 1054 to 1093, infra.

The present disclosure further provides preparations comprising a TBM or conjugate of the disclosure. Examples of preparations are described in Section 6.10 and specific embodiments 508 to 565 and 1094 to 1151, infra.

Pharmaceutical compositions comprising the TBMs and ADCs of the disclosure are also provided. Examples of pharmaceutical compositions are described in Section 6.11 and specific embodiments 566 and 1152, infra.

Further provided herein are methods of using the TBMs, the ADCs, and the pharmaceutical compositions of the disclosure, for example for treating proliferative conditions (e.g., cancers), on which the TAAs are expressed. Exemplary methods are described in Section 6.12 and specific embodiments 567 to 568 and 1153 to 1155, infra.

The disclosure further provides methods of using the TBMs, the ADCs, and the pharmaceutical compositions of the disclosure in combination with other agents and therapies. Exemplary agents, therapies, and methods of combination therapy are described in Section 6.13 and specific embodiments 569 and 1156 to 1168, infra.

### 5. BRIEF DESCRIPTION OF THE FIGURES

FIGS. 1A-1U: Exemplary TBM configurations. FIG. 1A illustrates components of the exemplary TBM configurations illustrated in FIGS. 1B-1Z. Not all regions connecting the different domains of each chain are illustrated (e.g., the linker connecting the VH and VL domains of an scFv, the hinge connecting the CH2 and CH3 domains of an Fc, etc., are omitted). FIGS. 1B-1O and 1V-Z illustrate trivalent TBMs; FIGS. 1P-1R illustrate tetravalent TBMs; FIG. 1S illustrates a pentavalent TBM, and FIGS. 1T-1U illustrate hexavalent TBMs.

FIG. 2: Schematics of the bispecific and trispecific constructs of Example 1.

FIGS. 3A-3D: Results of a re-directed T cell cytotoxicity assay using human CD-19-expressing Nalm6 target cells and a bispecific construct targeting human CD3 and human growth hormone, a bispecific construct targeting human CD3 and human CD19, and a TBM targeting human CD3, human CD2 and human CD19. FIG. 3A-3B: results obtained using pan T effector cells from a first donor after 24 hour (FIG. 3A) and 48 hour (FIG. 3B) incubations. FIG. 3C-3D: results obtained using pan T effector cells from a second donor after 24 hour (FIG. 3C) and 48 hour (FIG. 3D) incubations.

FIGS. 4A-4B: Results of a re-directed T cell cytotoxicity assay using human CD-19-expressing Daudi target cells and a bispecific construct targeting human CD3 and human growth hormone, a bispecific construct targeting human CD3 and human CD19, and a TBM targeting human CD3, human CD2 and human CD19. FIG. 4A: results obtained using pan T effector cells from a first donor after a 48-hour incubation. FIG. 4B: results obtained using pan T effector cells from a second donor after a-48 hour incubation.

FIG. 5: Results of a re-directed T cell cytotoxicity assay using negative control human K562 target cells and a bispecific construct targeting human CD3 and human growth hormone, a bispecific construct targeting human CD3 and human CD19, and a TBM targeting human CD3, human CD2 and human CD19.

FIG. 6A-6E: Results of a re-directed T cell cytotoxicity assay using human CD-19-expressing Nalm6 target cells and a bispecific construct targeting human CD3 and human growth hormone, a bispecific construct targeting human CD3 and human CD19, and a TBM targeting human CD3, human CD2 and human CD19. FIG. 6A-6E show results obtained using pan T effector cells from five different donors.

FIG. 7: Results of a re-directed T cell cytotoxicity assay using human CD-19-expressing Nalm6 target cells and a bispecific construct targeting human CD3 and human growth hormone, a bispecific construct targeting human CD3 and human CD19, and a TBM targeting human CD3, human CD2 and human CD19. FIG. 7 shows results obtained using human cryopreserved PBMCs from a patient diagnosed with acute myeloid leukemia.

FIGS. 8A-8D: Results of a re-directed T cell cytotoxicity assay using human CD-19-expressing Nalm6 target cells and a bispecific construct targeting human CD3 and human growth hormone, a bispecific construct targeting human CD3 and human CD19, and a TBM targeting human CD3, human CD2 and human CD19. FIG. 8A-8D show results obtained from target cell rechallenges at 48 hours, 72 hours, 96 hours, and 120 hours, respectively.

FIG. 9A-9B: Cell proliferation assay results. CD4+ sorted cells are shown in FIG. 9A and CD8+ sorted cells are shown in FIG. 9B.

FIGS. 10A-10F: Results of a cytokine release assay using human CD-19-expressing Nalm6 target cells and a bispecific construct targeting human CD3 and human growth hormone, a bispecific construct targeting human CD3 and human CD19, and a TBM targeting human CD3, human CD2 and human CD19. FIG. 10A: IFNγ; FIG. 10B: TNFα; FIG. 10C: IL2; FIG. 10D: IL10; FIG. 10E: IL6; FIG. 10F: figure legend.

FIG. 11: Results of a granzyme B ELISpot assay using human CD-19-expressing Nalm6 target cells and a bispecific construct targeting human CD3 and human growth hormone, a bispecific construct targeting human CD3 and human CD19, and a TBM targeting human CD3, human CD2 and human CD19.

FIGS. 12A-C: Schematics of the trispecific constructs of Example 2. FIG. 12A: AB2-1;

FIG. 12B: AB2-2; FIG. 12C: AB2-3.

FIG. 13: Results of a re-directed T cell cytotoxicity assay using human CD-19-expressing Nalm6 target cells and TBMs targeting human CD3, human CD2 and human CD19 (Example 2).

FIG. 14: Results of a re-directed T cell cytotoxicity assay using human CD-19-expressing Nalm6 target cells and and a bispecific construct targeting human CD3 and human growth hormone, a bispecific construct targeting human CD3 and human CD19, and a TBM targeting human CD3, human CD2 and human CD19 (Example 2). FIG. 14 shows results obtained using cynomolgus monkey pan T effector cells.

FIG. 15: Results of a NFAT activation assay using TBMs targeting human CD3, human CD2 and human CD19 (Example 2).

FIGS. 16A-E: Schematics of the trispecific constructs of Example 3. FIG. 16A: AB3-1;

FIG. 16B: AB3-2; FIG. 16C: AB3-3; FIG. 16D: AB3-4; FIG. 16E: AB3-5.

FIG. 17: Results of a re-directed T cell cytotoxicity assay using human CD-19-expressing Nalm6 target cells and TBMs targeting human CD3, human CD2 and human CD19 (Example 3).

FIG. 18: Results of a NFAT activation assay using TBMs targeting human CD3, human CD2 and human CD19 (Example 3).

FIGS. 19A-C: Schematics of the trispecific constructs of Example 4. FIG. 19A: AB4-1;

FIG. 19B: AB4-2; FIG. 19C: AB4-3.

FIG. 20: Results of a re-directed T cell cytotoxicity assay using human CD-19-expressing Nalm6 target cells and TBMs targeting human CD3, human CD2 and human CD19 (Example 4).

FIG. 21: Results of a NFAT activation assay using TBMs targeting human CD3, human CD2 and human CD19 (Example 4).

FIGS. 22A-22D: Results of a cytokine release assay using TBMs targeting human CD3, human CD2 and human CD19 (Example 4). FIG. 22A: IL-2; FIG. 22B: TNFα; FIG. 22C: IFNγ;

FIG. 22D: figure legend

FIGS. 23A-23B: Schematics of the bispecific and trispecfic constructs of Example 5. FIG. 23A: bispecific construct; FIG. 23B: trispecfic construct.

FIG. 24: Results of a re-directed T cell cytotoxicity assay using human CD-19-expressing Nalm6 target cells and either bispecific constructs targeting human CD3 and human CD19 or TBMs targeting human CD3, human CD2 and human CD19 (Example 5).

FIGS. 25A-25B: Results of a cytokine release assay using human CD-19-expressing Nalm6 target cells and bispecific constructs targeting human CD3 and human CD19, and TBMs targeting human CD3, human CD2 and human CD19 (Example 5). FIG. 25A shows show results for constructs having CD3 binding arms corresponding to BMA031 and FIG. 25B shows results for constructs having CD3 binding arms corresponding to OKT3. Results for trispecific constructs are are shown in black; results for bispecific constructs are shown in gray.

FIGS. 26A-26B: Schematics of the bispecific and trispecfic constructs of Example 6. FIG. 26A: bispecific construct; FIG. 26B: trispecfic construct.

FIG. 27: Results of a re-directed T cell cytotoxicity assay using human Her2-expressing HCC1954 target cells and a bispecific construct targeting human CD3 and human Her2 or a TBM targeting human CD3, human CD2 and human Her2 (Example 6).

FIG. 28: Results of a cytokine release assay using human Her2-expressing HCC1954 target cells and a bispecific construct targeting human CD3 and human Her2, and TBMs targeting human CD3, human CD2 and either human Her2 or GH (Example 6).

FIGS. 29A-B: Schematics of the bispecific and trispecfic constructs of Example 7. FIG. 29A: bispecific construct; FIG. 29B: trispecfic construct.

FIG. 30: Results of a re-directed T cell cytotoxicity assay using human mesothelin-expressing OVCAR8 target cells and a bispecific construct targeting human CD3 and human mesothelin or a TBM targeting human CD3, human CD2 and human mesothelin (Example 7).

FIG. 31: Results of a cytokine release assay using human Mesothelin-expressing Ovcar8 target cells and a bispecific construct targeting human CD3 and human mesothelin (MSLN), and TBMs targeting human CD3, human CD2 and either human mesothelin or GH (Example 6).

FIG. 32: Schematics of the trispecfic construct of Example 8.

### 6. DETAILED DESCRIPTION

### 6.1. Definitions

As used herein, the following terms are intended to have the following meanings:

Antigen-binding module: The term “antigen-binding module” or “ABM” as used herein refers to a portion of a TBM of the disclosure that has the ability to bind to an antigen non-covalently, reversibly and specifically. An ABM may be immunoglobulin- or non-immunoglobulin-based. As used herein, the terms “ABM1” and “CD2 ABM” (and the like) refers to an ABM that binds specifically to CD2, the terms “ABM2” and “TCR ABM” (and the like) refers to an ABM that binds specifically to a component of a TCR complex, and the term “ABM3” and “TAA ABM” (and the like) refer to an ABM that binds specifically to a tumor-associated antigen. The terms ABM1, ABM2, and ABM3 are used merely for convenience and are not intended to convey any particular configuration of a TBM. In some embodiments, a TCR ABM binds to CD3 (referred to herein a “CD3 ABM” or the like). Accordingly, disclosures relating to ABM2 and TCR ABMs are also applicable to CD3 ABMs.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies of the disclosure). The antibodies can be of any isotype/class (e.g., IgG, IgE, IgM, IgD, IgA and IgY) or subclass (e.g., IgG1, IgG2, IgG3, IgG4, IgA1 and IgA2).

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminus is a variable region and at the C-terminus is a constant region; the CH3 and CL domains actually comprise the carboxy-terminus of the heavy and light chain, respectively.

Antibody fragment: The term “antibody fragment” of an antibody as used herein refers to one or more portions of an antibody. In some embodiments, these portions are part of the contact domain(s) of an antibody. In some other embodiments, these portion(s) are antigen-binding fragments that retain the ability of binding an antigen non-covalently, reversibly and specifically, sometimes referred to herein as the “antigen-binding fragment”, “antigen-binding fragment thereof,” “antigen-binding portion”, and the like. Examples of binding fragments include, but are not limited to, single-chain Fvs (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Thus, the term “antibody fragment” encompasses both proteolytic fragments of antibodies (e.g., Fab and F(ab)2 fragments) and engineered proteins comprising one or more portions of an antibody (e.g., an scFv).

Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136). Antibody fragments can be grafted into scaffolds based on polypeptides such as Fibronectin type III (Fn3) (see U.S. Pat. No. 6,703,199, which describes fibronectin polypeptide monobodies).

Antibody fragments can be incorporated into single chain molecules comprising a pair of tandem Fv segments (for example, VH-CH1-VH-CH1) which, together with complementary light chain polypeptides (for example, VL-VC-VL-VC), form a pair of antigen-binding regions (Zapata et al., 1995, Protein Eng. 8:1057-1062; and U.S. Pat. No. 5,641,870).

Antigen-binding domain: The term “antigen-binding domain” refers a portion of a molecule that has the ability to bind to an antigen non-covalently, reversibly and specifically. Exemplary antigen-binding domains include antigen-binding fragments and portions of both immunoglobulin and non-immunoglobulin based scaffolds that retain the ability of binding an antigen non-covalently, reversibly and specifically. As used herein, the term “antigen-binding domain” encompasses antibody fragments that retain the ability of binding an antigen non-covalently, reversibly and specifically.

Half Antibody: The term “half antibody” refers to a molecule that comprises at least one ABM or ABM chain and can associate with another molecule comprising an ABM or ABM chain through, e.g., a disulfide bridge or molecular interactions (e.g., knob-in-hole interactions between Fc heterodimers). A half antibody can be composed of one polypeptide chain or more than one polypeptide chains (e.g., the two polypeptide chains of a Fab). In a preferred embodiment, a half-antibody comprises an Fc region.

An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, wherein said VL and VH domains form an ABM. Yet another example of a half antibody is a polypeptide comprising an scFv domain, a CH2 domain and a CH3 domain.

A half antibody might include more than one ABM, for example a half-antibody comprising (in N- to C-terminal order) an scFv domain, a CH2 domain, a CH3 domain, and another scFv domain.

Half antibodies might also include an ABM chain that when associated with another ABM chain in another half antibody forms a complete ABM.

Thus, a TBM can comprise one, more typically two, or even more than two half antibodies, and a half antibody can comprise one or more ABMs or ABM chains.

In some TBMs, a first half antibody will associate, e.g., heterodimerize, with a second half antibody. In other TBMs, a first half antibody will be covalently linked to a second half antibody, for example through disulfide bridges or chemical crosslinking. In yet other TBMs, a first half antibody will associate with a second half antibody through both covalent attachments and non-covalent interactions, for example disulfide bridges and knob-in-hole interactions.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, Md. (“Kabat” numbering scheme), Al-Lazikani et al., 1997, JMB 273:927-948 (“Chothia” numbering scheme) and ImMunoGenTics (IMGT) numbering (Lefranc, 1999, The Immunologist 7:132-136 (1999); Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

Single Chain Fv or scFv: The term “single-chain Fv” or “scFv” as used herein refers to antibody fragments comprise the VH and VL domains of an antibody, wherein these domains are present in a single polypeptide chain. Preferably, the Fv polypeptide further comprises a polypeptide linker between the VH and VL domains which enables the scFv to form the desired structure for antigen-binding. For a review of scFv see Plückthun in The Pharmacology of Monoclonal Antibodies, vol. 113, Rosenburg and Moore eds., (1994) Springer-Verlag, New York, pp. 269-315.

Diabody: The term “diabody” as used herein refers to small antibody fragments with two antigen-binding sites, typically formed by pairing of scFv chains. Each scFv comprises a heavy chain variable domain (VH) connected to a light chain variable domain (VL) in the same polypeptide chain (VH-VL, where the VH is either N-terminal or C-terminal to the VL). Unlike a typical scFv in which the VH and VL are separated by a linker that allows the VH and VL on the same polypeptide chain to pair and form an antigen-binding domain, diabodies typically comprise a linker that is too short to allow pairing between the VH and VL domains on the same chain, forcing the VH and VL domains to pair with the complementary domains of another chain and create two antigen-binding sites. Diabodies are described more fully in, for example, EP 404,097; WO 93/11161; and Hollinger et al., 1993, Proc. Natl. Acad. Sci. USA 90:6444-6448.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. By way of example and not limitation, the VH and VL can come together in any configuration described herein to form a half antibody, or can each be present on a separate half antibody and come together to form an antigen binding domain when the separate half antibodies associate, for example to form a TBM of the disclosure. When present on a single polypeptide chain (e.g., a scFv), the VH and be N-terminal or C-terminal to the VL.

Multispecific binding molecules: The term “multispecific binding molecules” refers to molecules that specifically bind to at least two antigens and comprise two or more antigen-binding domains. The antigen-binding domains can each independently be an antibody fragment (e.g., scFv, Fab, nanobody), a ligand, or a non-antibody derived binder (e.g., fibronectin, Fynomer, DARPin).

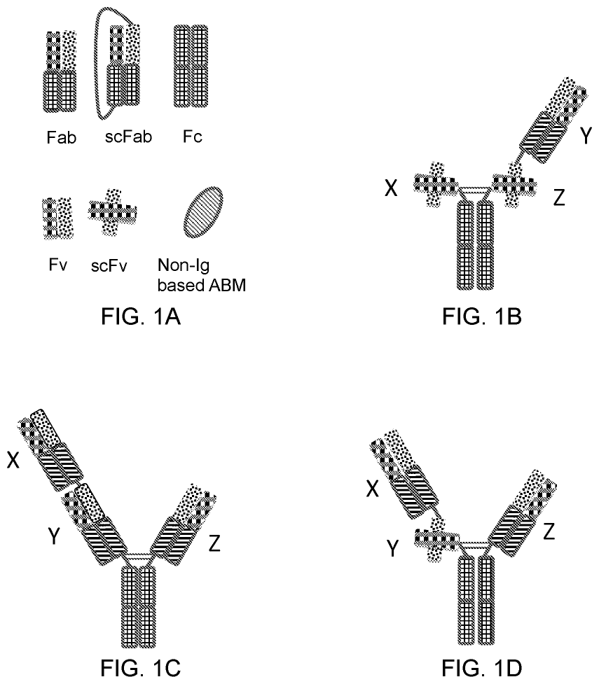

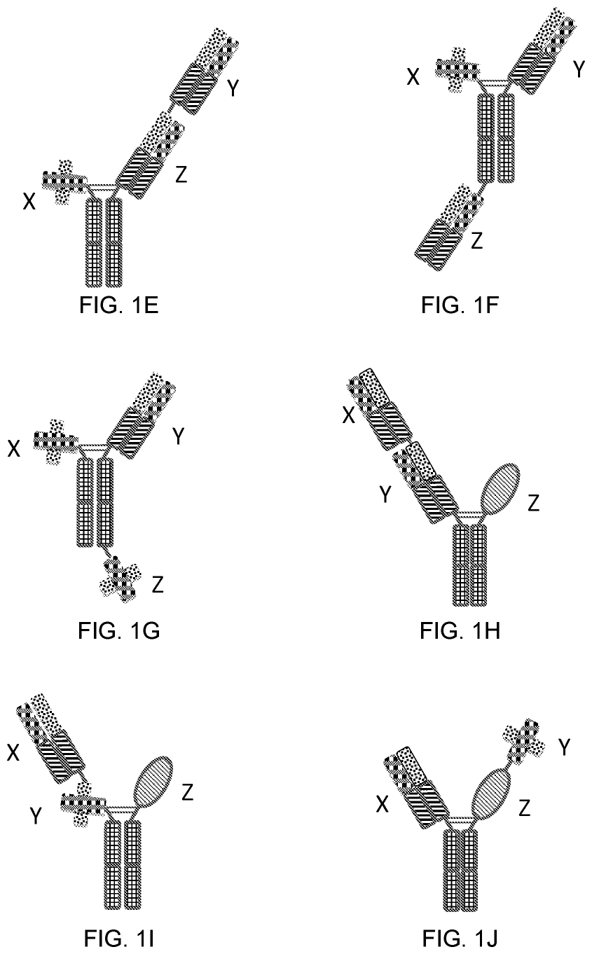

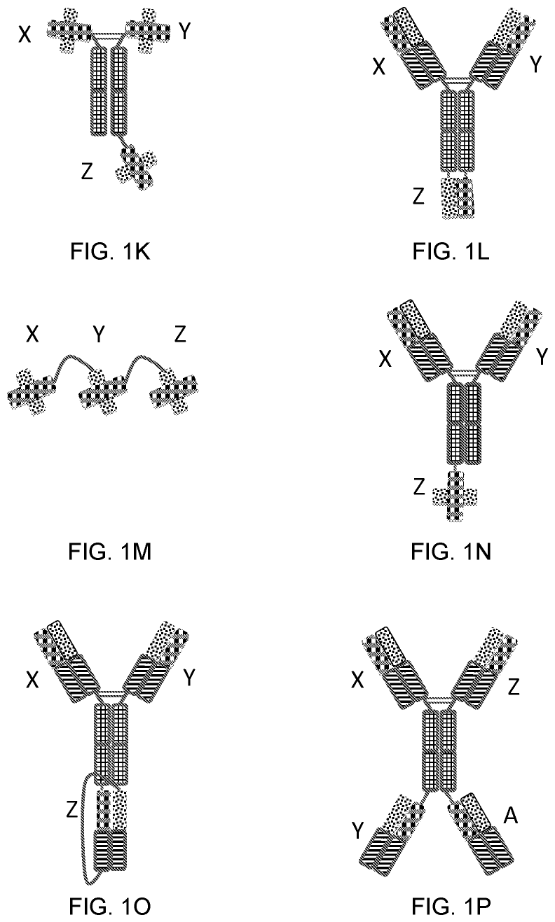

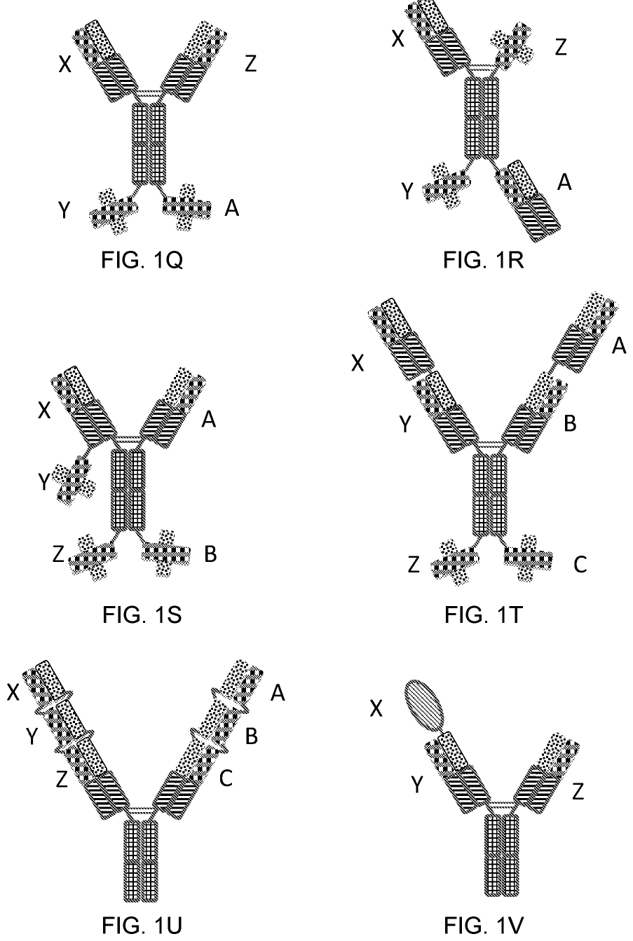

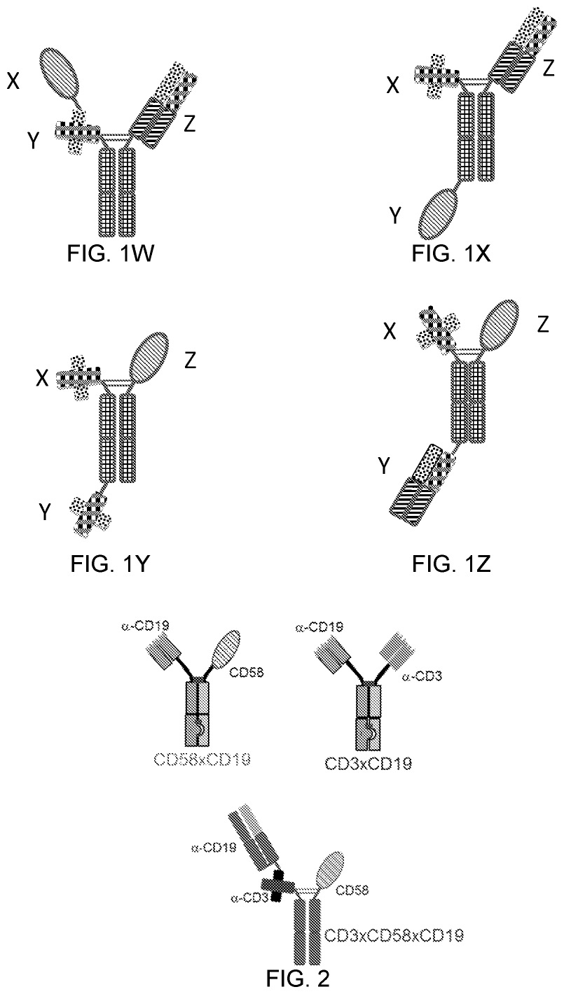

Trispecific binding molecules: The term “trispecific binding molecules” or “TBMs” refers to molecules that specifically bind to three antigens and comprise three or more antigen-binding domains. The TBMs of the disclosure comprise at least one antigen-binding domain which is specific for a component of a TCR complex, at least one antigen-binding domain which is specific for CD2, and at least one antigen-binding domain which is specific for a TAA. The antigen-binding domains can each independently be an antibody fragment (e.g., scFv, Fab, nanobody), a ligand, or a non-antibody derived binder (e.g., fibronectin, Fynomer, DARPin). Representative TBMs are illustrated in FIG. 1. TBMs can comprise one, two, three, four or even more polypeptide chains. For example, the TBM illustrated in FIG. 1M comprises a single polypeptide chain comprising three scFvs connected by ABM linkers one a single polypeptide chain. The TBM illustrated in FIG. 1K comprises two polypeptide chains comprising three scFvs connected by, inter alia, an Fc domain. The TBM illustrated in FIG. 1J comprises three polypeptide chains forming an scFv, a ligand, and a Fab connected by, inter alia, an Fc domain. The TBM illustrated in FIG. 1C comprises four polypeptide chains forming three Fabs connected by, inter alia, an Fc domain. The TBM illustrated in FIG. 1T comprises 6 polypeptide chains forming four Fabs and two scFvs connected by, inter alia, an Fc domain.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of a TBM of the disclosure, separate ABMs (or chains of an ABM) can be through peptide linker sequences. In the context of a nucleic acid encoding a fusion protein, such as a a polypeptide chain of a TBM of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Associated: The term “associated” in the context of a TBM refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional TBM in which ABM1, ABM2 and ABM3 can bind their respective targets. Examples of associations that might be present in a TBM of the disclosure include (but are not limited to) associations between Fc regions in an Fc domain (homodimeric or, more preferably, heterodimeric as described in Section 6.3.1.5), associations between VH and VL regions in a Fab or Fv, and associations between CH1 and CL in a Fab.

ABM chain: Individual ABMs can exist as one (e.g., in the case of an scFv) polypeptide chain or form through the association of more than one polypeptide chains (e.g., in the case of a Fab). As used herein, the term “ABM chain” refers to all or a portion of an ABM that exists on a single polypeptide chain. The use of the term “ABM chain” is intended for convenience and descriptive purposes only and does not connote a particular configuration or method of production.

Host cell or recombinant host cell: The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A host cell may carry the heterologous nucleic acid transiently, e.g., on an extrachromosomal heterologous expression vector, or stably, e.g., through integration of the heterologous nucleic acid into the host cell genome. For purposes of expressing a TBM of the disclosure, a host cell is preferably a cell line of mammalian origin or mammalian-like characteristics, such as monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Sequence identity: The term percent “identity,” in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or, in the case of a peptide or polypeptide, at least about 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, 1970, Adv. Appl. Math. 2:482c, by the homology alignment algorithm of Needleman and Wunsch, 1970, J. Mol. Biol. 48:443, by the search for similarity method of Pearson and Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., 2003, Current Protocols in Molecular Biology).

Two examples of algorithms that are suitable for determining percent sequence identity and sequence similarity are the BLAST and BLAST 2.0 algorithms, which are described in Altschul et al., 1977, Nuc. Acids Res. 25:3389-3402; and Altschul et al., 1990, J. Mol. Biol. 215:403-410, respectively. Software for performing BLAST analyses is publicly available through the National Center for Biotechnology Information.

The percent identity between two amino acid sequences can also be determined using the algorithm of Meyers and Miller, 1988, Comput. Appl. Biosci. 4:11-17, which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, 1970, J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Conservative Sequence Modifications: The term “conservative sequence modifications” refers to amino acid modifications that do not significantly affect or alter the binding characteristics of a TBM or a component thereof (e.g., an ABM or an Fc region). Such conservative modifications include amino acid substitutions, additions and deletions. Modifications can be introduced into a TBM of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a TBM of the disclosure can be replaced with other amino acid residues from the same side chain family and the altered TBM can be tested for, e.g., binding to target molecules and/or effective heterodimerization and/or effector function.

Mutation or modification: The terms “mutation” and “modification” in the context of a polypeptide as used herein can include substitution, addition or deletion of one or more amino acids.

Antibody Numbering Systems: In the present specification, the references to numbered amino acid residues in antibody domains are based on the EU numbering system unless otherwise specified (for example, in Tables 7B and 7C). This system was originally devised by Edelman et al., 1969, Proc. Nat'l Acad. Sci. USA 63:78-85 and is described in detail in Kabat et al., 1991, in Sequences of Proteins of Immunological Interest, US Department of Health and Human Services, NIH, USA.

dsFv: The term “dsFv” refers to disulfide-stabilized Fv fragments. In a dsFv, a VH and VL are connected by an interdomain disulfide bond. To generate such molecules, one amino acid each in the framework region of in VH and VL are mutated to a cysteine, which in turn form a stable interchain disulfide bond. Typically, position 44 in the VH and position 100 in the VL are mutated to cysteines. See Brinkmann, 2010, Antibody Engineering 181-189, D01:10.1007/978-3-642-01147-4_14. The term dsFv encompasses both what is known in the art a dsFv (a molecule in which the VH and VL are connected by an interchain disulfide bond but not a linker peptide) or scdsFv (a molecule in which the VH and VL are connected by a linker as well as an interchain disulfide bond).

Tandem of VH Domains: The term “a tandem of VH domains (or VHs)” as used herein refers to a string of VH domains, consisting of multiple numbers of identical VH domains of an antibody. Each of the VH domains, except the last one at the end of the tandem, has its C-terminus connected to the N-terminus of another VH domain with or without a linker. A tandem has at least 2 VH domains, and in particular embodiments of the TBMs of the disclosure has 3, 4, 5, 6, 7, 8, 9, or 10 VH domains. The tandem of VH can be produced by joining the encoding nucleic acids of each VH domain in a desired order using recombinant methods with or without a linker (e.g., as described in Section 6.3.3) that enables them to be made as a single polypeptide chain. The N-terminus of the first VH domain in the tandem is defined as the N-terminus of the tandem, while the C-terminus of the last VH domain in the tandem is defined as the C-terminus of the tandem.

Tandem of VL Domains: The term “a tandem of VL domains (or VLs)” as used herein refers to a string of VL domains, consisting of multiple numbers of identical VL domains of an antibody. Each of the VL domains, except the last one at the end of the tandem, has its C-terminus connected to the N-terminus of another VL with or without a linker. A tandem has at least 2 VL domains, and in particular embodiments of the TBMs of the disclosure has 3, 4, 5, 6, 7, 8, 9, or 10 VL domains. The tandem of VL can be produced by joining the encoding nucleic acids of each VL domain in a desired order using recombinant methods with or without a linker (e.g., as described in Section 6.3.3) that enables them to be made as a single polypeptide chain. The N-terminus of the first VL domain in the tandem is defined as the N-terminus of the tandem, while the C-terminus of the last VL domain in the tandem is defined as the C-terminus of the tandem.

Monovalent: The term “monovalent” as used herein in the context of an antigen-binding molecule refers to an antigen-binding molecule that has a single antigen-binding domain.

Bivalent: The term “bivalent” as used herein in the context of an antigen-binding molecule refers to an antigen-binding molecule that has two antigen-binding domains. The domains can be the same or different. Accordingly, a bivalent antigen-binding molecule can be monospecific or bispecific.

Trivalent: The term “trivalent” as used herein in the context of an antigen-binding molecule (e.g., a TBM) refers to an antigen-binding molecule that has three antigen-binding domains. The TBMs of the disclosure are trispecific and specifically bind to CD2, a component of a TCR complex and a TAA. Accordingly, the trivalent TBMs of the disclosure have at least three antigen-binding domains that each bind to a different antigen. Examples of trivalent TBMs of the disclosure are shown schematically in FIGS. 1B-1U.

Tetravalent: The term “tetravalent” as used herein in the context of an antigen-binding molecule (e.g., a TBM) refers to an antigen-binding molecule that has four antigen-binding domains. The TBMs of the disclosure are trispecific and specifically bind to CD2, a component of a TCR complex and a TAA. Accordingly, the tetravalent TBMs of the disclosure generally have two antigen-binding domains that bind to the same antigen (preferably the TAA) and two antigen-binding domains that each bind to a separate antigen (preferably CD2 and a component of a TCR complex). Examples of tetravalent TBMs of the disclosure are shown schematically in FIGS. 1P-1R.

Pentavalent: The term “pentavalent” as used herein in the context of an antigen-binding molecule (e.g., a TBM) refers to an antigen-binding molecule that has five antigen-binding domains. The TBMs of the disclosure are trispecific and specifically bind to CD2, a component of a TCR complex and a TAA. Accordingly, the pentavalent TBMs of the disclosure generally have either (a) two pairs of antigen-binding domains that each bind to the same antigen and a single antigen-binding domain that binds to the third antigen or (b) three antigen-binding domains that bind to the same antigen and two antigen-binding domains that each bind to a separate antigen. An example of a pentavalent TBM of the disclosure is shown schematically in FIG. 1S.

Hexavalent: The term “hexavalent” as used herein in the context of an antigen-binding molecule (e.g., a TBM) refers to an antigen-binding molecule that has six antigen-binding domains. The TBMs of the disclosure are trispecific and specifically bind to CD2, a component of a TCR complex and a TAA. The hexavalent TBMs of the disclosure generally have three pairs of antigen-binding domains that each bind to the same antigen, although different configurations (e.g., three antigen-binding domains that bind to the TAA, two antigen-binding domains that bind to a component of a TCR complex, and one antigen-binding domain that binds to CD2, or three antigen-binding domains that bind to the TAA, two antigen-binding domains that bind to CD2, and one antigen-binding domain that binds to a component of a TCR complex) are within the scope of the disclosure. Examples of hexavalent TBMs of the disclosure are shown schematically in FIGS. 1T-1U.

Specifically (or selectively) binds: The term “specifically (or selectively) binds” to an antigen or an epitope refers to a binding reaction that is determinative of the presence of a cognate antigen or an epitope in a heterogeneous population of proteins and other biologics. The binding reaction can be but need not be mediated by an antibody or antibody fragment, but can also be mediated by, for example, any type of ABM described in Section 6.2, such as a ligand, a DARPin, etc. An ABM of the disclosure typically also has a dissociation rate constant (KD) (koff/kon) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, or less than 10⁻⁹M, and binds to the target antigen with an affinity that is at least two-fold greater than its affinity for binding to a non-specific antigen (e.g., HSA). The term “specifically binds” does not exclude cross-species reactivity. For example, an antigen-binding module (e.g., an antigen-binding fragment of an antibody) that “specifically binds” to an antigen from one species may also “specifically bind” to that antigen in one or more other species. Thus, such cross-species reactivity does not itself alter the classification of an antigen-binding module as a “specific” binder. In certain embodiments, an antigen-binding module of the disclosure (e.g., ABM1, ABM2 and/or ABM3) that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus. In other embodiments, the antigen-binding module of the disclosure (e.g., ABM1, ABM2 and/or ABM3) does not have cross-species reactivity.

Monoclonal Antibody: The term “monoclonal antibody” as used herein refers to polypeptides, including antibodies, antibody fragments, molecules (including TBMs), etc. that are derived from the same genetic source.

Humanized: The term “humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin lo sequence. The humanized antibody optionally will also comprise at least a portion of an immunoglobulin constant region (Fc), typically that of a human immunoglobulin. For further details, see Jones et al., 1986, Nature 321:522-525; Riechmann et al., 1988, Nature 332:323-329; and Presta, 1992, Curr. Op. Struct. Biol. 2:593-596. See also the following review articles and references cited therein: Vaswani and Hamilton, 1998, Ann. Allergy, Asthma & Immunol. 1:105-115; Harris, 1995, Biochem. Soc. Transactions 23:1035-1038; Hurle and Gross, 1994, Curr. Op. Biotech. 5:428-433.

Human Antibody: The term “human antibody” as used herein includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., 2000, J Mol Biol 296, 57-86. The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Lazikani et al., 1997, J. Mol. Bio. 273:927 948; Kabat et al., 1991, Sequences of Proteins of Immunological Interest, 5th edit., NIH Publication no. 91-3242 U.S. Department of Health and Human Services; Chothia et al., 1987, J. Mol. Biol. 196:901-917; Chothia et al., 1989, Nature 342:877-883).

Human antibodies may include amino acid residues not encoded by human sequences (e.g., mutations introduced by random or site-specific mutagenesis in vitro or by somatic mutation in vivo, or a conservative substitution to promote stability or manufacturing). However, the term “human antibody”, as used herein, is not intended to include antibodies in which CDR sequences derived from the germline of another mammalian species, such as a mouse, have been grafted onto human framework sequences.

Chimeric Antibody: The term “chimeric antibody” (or antigen-binding fragment thereof) is an antibody molecule (or antigen-binding fragment thereof) in which (a) the constant region, or a portion thereof, is altered, replaced or exchanged so that the antigen-binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

Effector Function: The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which may be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody may be altered by altering, e.g., enhancing or reducing, the affinity of the antibody for an effector molecule such as an Fc receptor or a complement component. Binding affinity will generally be varied by modifying the effector molecule binding site, and in this case it is appropriate to locate the site of interest and modify at least part of the site in a suitable way. It is also envisaged that an alteration in the binding site on the antibody for the effector molecule need not alter significantly the overall binding affinity but may alter the geometry of the interaction rendering the effector mechanism ineffective as in non-productive binding. It is further envisaged that an effector function may also be altered by modifying a site not directly involved in effector molecule binding, but otherwise involved in performance of the effector function.

Recognize: The term “recognize” as used herein refers to an ABM that finds and interacts (e.g., binds) with its epitope.

Epitope: An epitope, or antigenic determinant, is a portion of an antigen recognized by an antibody or other antigen-binding moiety as described herein. An epitope can be linear or conformational.

Nucleic Acid: The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

Vector: The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

Binding Sequences: In reference to Tables 7, 8, 9, 11, 12 or 13 (including subparts thereof), the term “binding sequences” means an ABM having a full set of CDRs, a VH-VL pair, or an scFv set forth in that table.

VH-VL or VH-VL Pair: In reference to a VH-VL pair, whether on the same polypeptide chain or on different polypeptide chains, the terms “VH-VL” and “VH-VL pair” are used for convience and are not intended to convey any particular orientation, unless the context dictates otherwise. Thus, a scFv comprising a “VH-VL” or “VH-VL pair” can have the VH and VL domains in any orientation, for example the VH N-terminal to the VL or the VL N-terminal to the VH.

Polypeptide and Protein: The terms “polypeptide” and “protein” are used interchangeably herein to refer to a polymer of amino acid residues. The phrases also apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, binary tract cancer, bone cancer, endometrial cancer, eye cancer, fallopian tube cancer, genital tract cancers, large intestinal cancer, cancer of the meninges, oesophageal cancer, peritoneial cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like, e.g., any TAA-positive cancers of any of the foregoing types.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens (“TSAs”).

Treat, Treatment, Treating: As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more TBMs of the disclosure. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

### 6.2. Antigen Binding Modules

Typically, one or more ABMs of the TBMs of the disclosure comprise immunoglobulin-based antigen-binding domains, for example the sequences of antibody fragments or derivatives. These antibody fragments and derivatives typically include the CDRs of an antibody and can include larger fragments and derivatives thereof, e.g., Fabs, scFabs, Fvs, and scFvs.

6.2.1. Immunoglobulin Based Modules

6.2.1.1. Fabs

In certain aspects, an ABM of the disclosure is a Fab domain. Fab domains can be produced by proteolytic cleavage of immunoglobulin molecules, using enzymes such as papain, or through recombinant expression. Fab domains typically comprise a CH1 domain attached to a VH domain which pairs with a CL domain attached to a VL domain.

In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the TBMs of the disclosure, it is advantageous to use Fab heterodimerization strategies to permit the correct association of Fab domains belonging to the same ABM and minimize aberrant pairing of Fab domains belonging to different ABMs. For example, the Fab heterodimerization strategies shown in Table 1 below can be used:

TABLE 1
Fab Heterodimerization Strategies

| Name | STRATEGY | VH | CH1 | VL | CL | REFERENCE |
|---|---|---|---|---|---|---|
| F1 | CrossMabCH1- | WT | CL domain | WT | CH1 domain | Schaefer et al., 2011, |
| | CL | | | | | Cancer Cell 2011; |
| | | | | | | 20:472-86; |
| | | | | | | PMID:22014573. |
| F2 | orthogonal Fab | 39K, | H172A, | 1R, 38D, | L135Y, | Lewis et al., 2014, Nat |
| | VHVRD1CH1CRD2- | 62E | F174G | (36F) | S176W | Biotechnol 32:191-8 |
| | VLVRD1CλCRD2 | | | | | |
| F3 | orthogonal Fab | 39Y | WT | 38R | WT | Lewis et al., 2014, Nat |
| | VHVRD2CH1wt- | | | | | Biotechnol 32:191-8 |
| | VLVRD2Cλwt | | | | | |
| F4 | TCR CαCβ | 39K | TCR Cα | 38D | TCR Cβ | Wu et al., 2015, MAbs |
| | | | | | | 7:364-76 |
| F5 | CR3 | WT | T192E | WT | N137K, | Golay at al., 2016, J |
| | | | | | S114A | Immunol 196:3199- |
| | | | | | | 211. |
| F6 | MUT4 | WT | L143Q, | WT | V133T, | Golay at al., 2016, J |
| | | | S188V | | S176V | Immunol 196:3199- |
| | | | | | | 211. |
| F7 | DuetMab | WT | F126C | WT | S121C | Mazor et al., 2015, |
| | | | | | | MAbs 7:377-89; Mazor |
| | | | | | | et al., 2015, MAbs |
| | | | | | | 7:461-669. |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121C in the CL domain (see, Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the α T cell receptor and substituting the CL domain with the β domain of the T cell receptor, and pairing these domain replacements with an additional charge-charge interaction between the VL and VH domains by introducing a 38D modification in the VL domain and a 39K modification in the VH domain.

ABMs of the disclosure can comprise a single chain Fab fragment, which is a polypeptide consisting of an antibody heavy chain variable domain (VH), an antibody constant domain 1 (CH1), an antibody light chain variable domain (VL), an antibody light chain constant domain (CL) and a linker. In some embodiments, the antibody domains and the linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. The linker can be a polypeptide of at least 30 amino acids, preferably between 32 and 50 amino acids. The single chain Fab domains are stabilized via the natural disulfide bond between the CL domain and the CH1 domain.

In an embodiment the antibody domains and the linker in the single chain Fab fragment have one of the following orders in N-terminal to C-terminal direction: a) VH-CH1-linker-VL-CL, or b) VL-CL-linker-VH-CH1, more preferably VL-CL-linker-VH-CH1.

In another embodiment the antibody domains and the linker in the single chain Fab fragment have one of the following orders in N-terminal to C-terminal direction: a) VH-CL-linker-VL-CH1 or b) VL-CH1-linker-VH-CL.

Optionally in the single chain Fab fragment, additionally to the natural disulfide bond between the CL-domain and the CH1 domain, also the antibody heavy chain variable domain (VH) and the antibody light chain variable domain (VL) are disulfide stabilized by introduction of a disulfide bond between the following positions: i) heavy chain variable domain position 44 to light chain variable domain position 100, ii) heavy chain variable domain position 105 to light chain variable domain position 43, or iii) heavy chain variable domain position 101 to light chain variable domain position 100 (numbering according to EU index of Kabat).

Such further disulfide stabilization of single chain Fab fragments is achieved by the introduction of a disulfide bond between the variable domains VH and VL of the single chain Fab fragments. Techniques to introduce unnatural disulfide bridges for stabilization for a single chain Fv are described e.g. in WO 94/029350, Rajagopal et al., 1997, Prot. Engin. 10:1453-59; Kobayashi et al., 1998, Nuclear Medicine & Biology, 25:387-393; and Schmidt, et al., 1999, Oncogene 18:1711-1721. In one embodiment the optional disulfide bond between the variable domains of the single chain Fab fragments is between heavy chain variable domain position 44 and light chain variable domain position 100. In one embodiment the optional disulfide bond between the variable domains of the single chain Fab fragments is between heavy chain variable domain position 105 and light chain variable domain position 43 (numbering according to EU index of Kabat).

6.2.1.2. scFvs

Single chain Fv or “scFv” antibody fragments comprise the VH and VL domains of an antibody in a single polypeptide chain, are capable of being expressed as a single chain polypeptide, and retain the specificity of the intact antibody from which it is derived. Generally, the scFv polypeptide further comprises a polypeptide linker between the VH and VL domain that enables the scFv to form the desired structure for target binding. Examples of linkers suitable for connecting the VH and VL chains of an scFV are the ABM linkers identified in Section 6.3.3, for example any of the linkers designated L1 through L54.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

To create an scFv-encoding nucleic acid, the VH and VL-encoding DNA fragments are operably linked to another fragment encoding a linker, e.g., encoding any of the ABM linkers described in Section 6.3.3 (such as the amino acid sequence (Gly4 Ser)3) (SEQ ID NO: 1), such that the VH and VL sequences can be expressed as a contiguous single-chain protein, with the VL and VH regions joined by the flexible linker (see e.g., Bird et al., 1988, Science 242:423-426; Huston et al., 1988, Proc. Natl. Acad. Sci. USA 85:5879-5883; McCafferty et al., 1990, Nature 348:552-554).

6.2.1.3. Other Immunoglobulin-Based Modules

TBMs of the disclosure can also comprise ABMs having an immunoglobulin format which is other than Fab or scFv, for example Fv, dsFv, (Fab′)2, a single domain antibody (SDAB), a VH or VL domain, or a camelid VHH domain (also called a nanobody).

An ABM can be a single domain antibody composed of a single VH or VL domain which exhibits sufficient affinity to the target. In a specific embodiment, the single domain antibody is a camelid VHH domain (see, e.g., Riechmann, 1999, Journal of Immunological Methods 231:25-38; WO 94/04678).

6.2.2. Non-Immunoglobulin Based Modules

In certain embodiments, one or more of the ABMs of the disclosure are derived from non-antibody scaffold proteins (including, but not limited to, designed ankyrin repeat proteins (DARPins), Avimers (short for avidity multimers), Anticalin/Lipocalins, Centyrins, Kunitz domains, Adnexins, Affilins, Affitins (also known as Nonfitins), Knottins, Pronectins, Versabodies, Duocalins, and Fynomers), ligands, receptors, cytokines or chemokines.

Non-immunoglobulin scaffolds that can be used in the TBMs of the disclosure include those listed in Tables 3 and 4 of Mintz and Crea, 2013, Bioprocess International 11(2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18. The contents of Tables 3 and 4 of Mintz and Crea, 2013, Bioprocess International 11(2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18 (collectively, “Scaffold Disclosures”) are incorporated by reference herein. In a particular embodiment, the Scaffold Disclosures are incorporated by reference for what they disclose relating to Adnexins. In another embodiment, the Scaffold Disclosures are incorporated by reference for what they disclose relating to Avimers. In another embodiment, the Scaffold Disclosures are incorporated by reference for what they disclose relating to Affibodies. In yet another embodiment, the Scaffold Disclosures are incorporated by reference for what they disclose relating to Anticalins. In yet another embodiment, the Scaffold Disclosures are incorporated by reference for what they disclose relating to DARPins. In yet another embodiment, the Scaffold Disclosures are incorporated by reference for what they disclose relating to Kunitz domains. In yet another embodiment, the Scaffold Disclosures are incorporated by reference for what they disclose relating to Knottins. In yet another embodiment, the Scaffold Disclosures are incorporated by reference for what they disclose relating to Pronectins. In yet another embodiment, the Scaffold Disclosures are incorporated by reference for what they disclose relating to Nanofitins. In yet another embodiment, the Scaffold Disclosures are incorporated by reference for what they disclose relating to Affilins. In yet another embodiment, the Scaffold Disclosures are incorporated by reference for what they disclose relating to Adnectins. In yet another embodiment, the Scaffold Disclosures are incorporated by reference for what they disclose relating to ABDs. In yet another embodiment, the Scaffold Disclosures are incorporated by reference for what they disclose relating to Adhirons. In yet another embodiment, the Scaffold Disclosures are incorporated by reference for what they disclose relating to Affimers. In yet another embodiment, the Scaffold Disclosures are incorporated by reference for what they disclose relating to Alphabodies. In yet another embodiment, the Scaffold Disclosures are incorporated by reference for what they disclose relating to Armadillo Repeat Proteins. In yet another embodiment, the Scaffold Disclosures are incorporated by reference for what they disclose relating to Atrimers/Tetranectins. In yet another embodiment, the Scaffold Disclosures are incorporated by reference for what they disclose relating to Obodies/OB-folds. In yet another embodiment, the Scaffold Disclosures are incorporated by reference for what they disclose relating to Centyrins. In yet another embodiment, the Scaffold Disclosures are incorporated by reference for what they disclose relating to Repebodies. In yet another embodiment, the Scaffold Disclosures are incorporated by reference for what they disclose relating to Anticalins. In yet another embodiment, the Scaffold Disclosures are incorporated by reference for what they disclose relating to Atrimers. In yet another embodiment, the Scaffold Disclosures are incorporated by reference for what they disclose relating to bicyclic peptides. In yet another embodiment, the Scaffold Disclosures are incorporated by reference for what they disclose relating to cys-knots. In yet another embodiment, the Scaffold Disclosures are incorporated by reference for what they disclose relating to Fn3 scaffolds (including Adnectins, Centryrins, Pronectins, and Tn3).

In an embodiment, an ABM of the disclosure can be a designed ankyrin repeat protein (“DARPin”). DARPins are antibody mimetic proteins that typically exhibit highly specific and high-affinity target protein binding. They are typically genetically engineered and derived from natural ankyrin proteins and consist of at least three, usually four or five repeat motifs of these proteins. Their molecular mass is about 14 or 18 kDa (kilodaltons) for four- or five-repeat DARPins, respectively. Examples of DARPins can be found, for example in U.S. Pat. No. 7,417,130. Multispecific binding molecules comprising DARPin binding modules and immunoglobulin-based binding modules are disclosed in, for example, U.S. Publication No. 2015/0030596 A1.

In another embodiment, an ABM of the disclosure can be an Affibody. An Affibody is well known in the art and refers to affinity proteins based on a 58 amino acid residue protein domain, derived from one of the IgG binding domain of staphylococcal protein A.

In another embodiment, an ABM of the disclosure can be an Anticalin. Anticalins are well known in the art and refer to another antibody mimetic technology, wherein the binding specificity is derived from Lipocalins. Anticalins may also be formatted as dual targeting protein, called Duocalins.

In another embodiment, an ABM of the disclosure can be a Versabody. Versabodies are well known in the art and refer to another antibody mimetic technology. They are small proteins of 3-5 kDa with >15% cysteines, which form a high disulfide density scaffold, replacing the hydrophobic core the typical proteins have.

Other non-immunoglobulin ABMs include “A” domain oligomers (also known as Avimers) (see for example, U.S. Patent Application Publication Nos. 2005/0164301, 2005/0048512, and 2004/017576), Fn3 based protein scaffolds (see for example, U.S. Patent Application Publication 2003/0170753), VASP polypeptides, Avian pancreatic polypeptide (aPP), Tetranectin (based on CTLD3), Affililin (based on yB-crystallin/ubiquitin), Knottins, SH3 domains, PDZ domains, Tendamistat, Neocarzinostatin, Protein A domains, Lipocalins, Transferrin, and Kunitz domains. In one aspect, ABMs useful in the construction of the TBMs of the disclosure comprise fibronectin-based scaffolds as exemplified in WO 2011/130324.

Moreover, in certain aspects, an ABM comprises a ligand binding domain of a receptor or a receptor binding domain of a ligand. For example, if the TAA is the EGF receptor, ABM3 can comprise a portion of EGF that binds EGFR, and if the TAA is the PDGF receptor, ABM3 can comprise a portion of PDGF that binds PDGF, and so on and so forth. In a specific embodiment, ABM1 is a CD2 ligand, in particular a CD58 moiety as described in Section 6.6.2. The respective binding domains of numerous ligand/receptor pairs are well known in the art, and thus can be readily selected and adapted for use in the TBMs of the disclosure.

### 6.3. Connectors

It is contemplated that the TBMs of the disclosure can in some instances include pairs of ABMs or ABM chains (e.g., the VH-CH1 or VL-CL component of a Fab) connected directly to one another, e.g., as a fusion protein without a linker. More preferably, the TBMs of the disclosure comprise connector moieties linking individual ABMs or ABM chains. The use of connector moieties can improve target binding, for example by increasing flexibility of the ABMs within a TBM and thus reducing steric hindrance. The ABMs can be connected to one another through, for example, Fc domains (each Fc domain representing a pair of associated Fc regions) and/or ABM linkers. The use of Fc domains will typically require the use of hinge regions as connectors of the ABMs or ABM chains for optimal antigen binding. Thus, the term “connector” encompasses, but is not limited to, Fc regions, Fc domains, hinge regions, and

Examples of Fc domains (formed by the pairing of two Fc regions), hinge regions and ABM linkers are described in Sections 6.3.1, 6.3.2, and 6.3.3, respectively.

6.3.1. Fc Domains

The TBMs of the disclosure can include an Fc domain derived from any suitable species. In one embodiment the Fc domain is derived from a human Fc domain.

The Fc domain may be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4.

The Fc domain comprises two polypeptide chains, each referred to as a heavy chain Fc region. The two heavy chain Fc regions dimerize to create the Fc domain. The two Fc regions within the Fc domain may be the same or different from one another. In a native antibody the Fc regions are typically identical, but for the purpose of producing multispecific binding molecules, e.g., the TBMs of the disclosure, the Fc regions might advantageously be different to allow for heterodimerization, as described in Section 6.3.1.5 below.

Typically each heavy chain Fc region comprises or consists of two or three heavy chain constant domains.

In native antibodies, the heavy chain Fc region of IgA, IgD and IgG is composed of two heavy chain constant domains (CH2 and CH3) and that of IgE and IgM is composed of three heavy chain constant domains (CH2, CH3 and CH4). These dimerize to create an Fc domain.

In the present disclosure, the heavy chain Fc region can comprise heavy chain constant domains from one or more different classes of antibody, for example one, two or three different classes.

In one embodiment the heavy chain Fc region comprises CH2 and CH3 domains derived from IgG1.

In one embodiment the heavy chain Fc region comprises CH2 and CH3 domains derived from IgG2.

In one embodiment the heavy chain Fc region comprises CH2 and CH3 domains derived from IgG3.

In one embodiment the heavy chain Fc region comprises CH2 and CH3 domains derived from IgG4.

In one embodiment the heavy chain Fc region comprises a CH4 domain from IgM. The IgM CH4 domain is typically located at the C-terminus of the CH3 domain.

In one embodiment the heavy chain Fc region comprises CH2 and CH3 domains derived from IgG and a CH4 domain derived from IgM.

It will be appreciated that the heavy chain constant domains for use in producing a heavy chain Fc region for the TBMs of the present disclosure may include variants of the naturally occurring constant domains described above. Such variants may comprise one or more amino acid variations compared to wild type constant domains. In one example the heavy chain Fc region of the present disclosure comprises at least one constant domain that varies in sequence from the wild type constant domain. It will be appreciated that the variant constant domains may be longer or shorter than the wild type constant domain. Preferably the variant constant domains are at least 60% identical or similar to a wild type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar. Exemplary Fc variants are described in Sections 6.3.1.1 through 6.3.1.5, infra.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the TBMs of the present disclosure do not comprise a tailpiece.

The Fc domains that are incorporated into the TBMs of the present disclosure may comprise one or more modifications that alter the functional properties of the proteins, for example, binding to Fc-receptors such as FcRn or leukocyte receptors (for example, as described in Section 6.3.1.1), binding to complement (for example as described in Section 6.3.1.2), modified disulfide bond architecture (for example as described in Section 6.3.1.3), or altered glycosylation patterns (for example as described in Section 6.3.1.4). The Fc domains can also be altered to include modifications that improve manufacturability of asymmetric TBMs, for example by allowing heterodimerization, which is the preferential pairing of non-identical Fc regions over identical Fc regions. Heterodimerization permits the production of TBMs in which different ABMs are connected to one another by an Fc domain containing Fc regions that differ in sequence. Examples of heterodimerization strategies are exemplified in Section 6.3.1.5 (and subsections thereof).

It will be appreciated that any of the modifications described in Sections 6.3.1.1 through 6.3.1.5 can be combined in any suitable manner to achieve the desired functional properties and/or combined with other modifications to alter the properties of the TBMs.

6.3.1.1. Fc Domains with Altered FcR Binding

The Fc domains of the TBMs of the disclosure may show altered binding to one or more Fc-receptors (FcRs) in comparison with the corresponding native immunoglobulin. The binding to any particular Fc-receptor may be increased or decreased. In one embodiment, the Fc domain comprises one or more modifications which alter its Fc-receptor binding profile.

Human cells can express a number of membrane bound FcRs selected from FcaR, FccR, FcγR, FcRn and glycan receptors. Some cells are also capable of expressing soluble (ectodomain) FcR (Fridman et al., 1993, J Leukocyte Biology 54: 504-512 for review). FcγR can be further divided by affinity of IgG binding (high/low) and biological effect (activating/inhibiting). Human FcγRI is widely considered to be the sole ‘high affinity’ receptor whilst all of the others are considered as medium to low. FcγRIIb is the sole receptor with ‘inhibitory’ functionality by virtue of its intracellular ITIM motif whilst all of the others are considered as ‘activating’ by virtue of ITAM motifs or pairing with the common FcγR-γchain. FcγRIIIb is also unique in that although activatory it associates with the cell via a GPI anchor. In total, humans express six “standard” FcγRs: FcγRI, FcγRIIa, FcγRIIb, FcγRIIc, FcγRIIIa FcγRIIIb. In addition to these sequences there are a large number of sequence or allotypic variants spread across these families. Some of these have been found to have important functional consequence and so are sometimes considered to be receptor sub-types of their own. Examples include FcγRIIa^{H134R}, FcγRIIb^{I190T}, FcγRIIIa^{F158V} and FcγRIIIb^{NA1}, FcγRIIIb^{NA2} FcγRIII^{SH}. Each receptor sequence has been shown to have different affinities for the 4 sub-classes of IgG: IgG1, IgG2, IgG3 and IgG4 (Bruhns, 1993, Blood 113:3716-3725). Other species have somewhat different numbers and functionality of FcγR, with the mouse system being the best studied to date and comprising of 4 FcγR, FcγRI FcγRIIb FcγRIII FcγRIV (Bruhns, 2012, Blood 119:5640-5649). Human FcγRI on cells is normally considered to be ‘occupied’ by monomeric IgG in normal serum conditions due to its affinity for IgG1/IgG3/IgG4 (about 10⁻⁸ M) and the concentration of these IgG in serum (about 10 mg/ml). Hence cells bearing FcγRI on their surface are considered to be capable for “screening” or “sampling” of their antigenic environment vicariously through the bound polyspecific IgG. The other receptors having lower affinities for IgG sub-classes (in the range of about 10⁻⁵-10⁻⁷ M) are normally considered to be “unoccupied.” The low affinity receptors are hence inherently sensitive to the detection of and activation by antibody involved immune complexes. The increased Fc density in an antibody immune complex results in increased functional affinity of binding avidity to low affinity FcγR. This has been demonstrated in vitro using a number of methods (Shields et al., 2001, J Biol Chem 276(9):6591-6604; Lux et al., 2013, J Immunol 190:4315-4323). It has also been implicated as being one of the primary modes of action in the use of anti-RhD to treat ITP in humans (Crow, 2008, Transfusion Medicine Reviews 22:103-116).

Many cell types express multiple types of FcγR and so binding of IgG or antibody immune complex to cells bearing FcγR can have multiple and complex outcomes depending upon the biological context. Most simply, cells can either receive an activatory, inhibitory or mixed signal. This can result in events such as phagocytosis (e.g., macrophages and neutrophils), antigen processing (e.g., dendritic cells), reduced IgG production (e.g., B-cells) or degranulation (e.g., neutrophils, mast cells). There are data to support that the inhibitory signal from FcγRIIb can dominate that of activatory signals (Proulx, 2010, Clinical Immunology 135:422-429).

FcRn has a crucial role in maintaining the long half-life of IgG in the serum of adults and children. The receptor binds IgG in acidified vesicles (pH<6.5) protecting the IgG molecule from degradation, and then releasing it at the higher pH of 7.4 in blood.

FcRn is unlike leukocyte Fc receptors, and instead, has structural similarity to MHC class I molecules. It is a heterodimer composed of a β₂-microglobulin chain, non-covalently attached to a membrane-bound chain that includes three extracellular domains. One of these domains, including a carbohydrate chain, together with β₂-microglobulin interacts with a site between the CH2 and CH3 domains of Fc. The interaction includes salt bridges made to histidine residues on IgG that are positively charged at pH<6.5. At higher pH, the His residues lose their positive charges, the FcRn-IgG interaction is weakened and IgG dissociates.

In one embodiment, a TBM of the disclosure comprises an Fc domain that binds to human FcRn.

In one embodiment, the Fc domain has an (e.g., one or two) Fc regions comprising a histidine residue at position 310, and preferably also at position 435. These histidine residues are important for human FcRn binding. In one embodiment, the histidine residues at positions 310 and 435 are native residues, i.e., positions 310 and 435 are not modified. Alternatively, one or both of these histidine residues may be present as a result of a modification.

The TBMs of the disclosure may comprise one or more Fc regions that alter Fc binding to FcRn. The altered binding may be increased binding or decreased binding.

In one embodiment, the TBM comprises an Fc domain in which at least one (and optionally both) Fc regions comprises one or more modifications such that it binds to FcRn with greater affinity and avidity than the corresponding native immunoglobulin.

In one embodiment, the Fc region is modified by substituting the threonine residue at position 250 with a glutamine residue (T250Q).

In one embodiment, the Fc region is modified by substituting the methionine residue at position 252 with a tyrosine residue (M252Y)

In one embodiment, the Fc region is modified by substituting the serine residue at position 254 with a threonine residue (S254T).

In one embodiment, the Fc region is modified by substituting the threonine residue at position 256 with a glutamic acid residue (T256E).

In one embodiment, the Fc region is modified by substituting the threonine residue at position 307 with an alanine residue (T307A).

In one embodiment, the Fc region is modified by substituting the threonine residue at position 307 with a proline residue (T307P).

In one embodiment, the Fc region is modified by substituting the valine residue at position 308 with a cysteine residue (V308C).

In one embodiment, the Fc region is modified by substituting the valine residue at position 308 with a phenylalanine residue (V308F).

In one embodiment, the Fc region is modified by substituting the valine residue at position 308 with a proline residue (V308P).

In one embodiment, the Fc region is modified by substituting the glutamine residue at position 311 with an alanine residue (Q311A).

In one embodiment, the Fc region is modified by substituting the glutamine residue at position 311 with an arginine residue (Q311R).

In one embodiment, the Fc region is modified by substituting the methionine residue at position 428 with a leucine residue (M428L).

In one embodiment, the Fc region is modified by substituting the histidine residue at position 433 with a lysine residue (H433K).

In one embodiment, the Fc region is modified by substituting the asparagine residue at position 434 with a phenylalanine residue (N434F).

In one embodiment, the Fc region is modified by substituting the asparagine residue at position 434 with a tyrosine residue (N434Y).

In one embodiment, the Fc region is modified by substituting the methionine residue at position 252 with a tyrosine residue, the serine residue at position 254 with a threonine residue, and the threonine residue at position 256 with a glutamic acid residue (M252Y/S254T/T256E).

In one embodiment, the Fc region is modified by substituting the valine residue at position 308 with a proline residue and the asparagine residue at position 434 with a tyrosine residue (V308P/N434Y).

In one embodiment, the Fc region is modified by substituting the methionine residue at position 252 with a tyrosine residue, the serine residue at position 254 with a threonine residue, the threonine residue at position 256 with a glutamic acid residue, the histidine residue at position 433 with a lysine residue and the asparagine residue at position 434 with a phenylalanine residue (M252Y/S254T/T256E/H433K/N434F).

It will be appreciated that any of the modifications listed above may be combined to alter FcRn binding.

In one embodiment, the TBM comprises an Fc domain in which one or both Fc regions comprise one or more modifications such that the Fc domain binds to FcRn with lower affinity and avidity than the corresponding native immunoglobulin.

In one embodiment, the Fc region comprises any amino acid residue other than histidine at position 310 and/or position 435.

The TBM of the disclosure may comprise an Fc domain in which one or both Fc regions comprise one or more modifications which increase its binding to FcγRIIb. FcγRIIb is the only inhibitory receptor in humans and the only Fc receptor found on B cells.

In one embodiment, the Fc region is modified by substituting the proline residue at position 238 with an aspartic acid residue (P238D).

In one embodiment, the Fc region is modified by substituting the glutamic acid residue at position 258 with an alanine residue (E258A).

In one embodiment, the Fc region is modified by substituting the serine residue at position 267 with an alanine residue (S267A).

In one embodiment, the Fc region is modified by substituting the serine residue at position 267 with a glutamic acid residue (S267E).

In one embodiment, the Fc region is modified by substituting the leucine residue at position 328 with a phenylalanine residue (L328F).

In one embodiment, the Fc region is modified by substituting the glutamic acid residue at position 258 with an alanine residue and the serine residue at position 267 with an alanine residue (E258A/S267A).

In one embodiment, the Fc region is modified by substituting the serine residue at position 267 with a glutamic acid residue and the leucine residue at position 328 with a phenylalanine residue (S267E/L328F).

It will be appreciated that any of the modifications listed above may be combined to increase FcγRIIb binding.

In one embodiment of the disclosure, TBMs are provided comprising Fc domains which display decreased binding to FcγR.

In one embodiment TBM comprises an Fc domain in which one or both Fc regions comprise one or more modifications that decrease Fc binding to FcγR.

The Fc domain can be derived from IgG1.

In one embodiment, the Fc region is modified by substituting the leucine residue at position 234 with an alanine residue (L234A).

In one embodiment, the Fc region is modified by substituting the leucine residue at position 235 with an alanine residue (L235A).

In one embodiment, the Fc region is modified by substituting the glycine residue at position 236 with an arginine residue (G236R).

In one embodiment, the Fc region is modified by substituting the asparagine residue at position 297 with an alanine residue (N297A) or a glutamine residue (N297Q).

In one embodiment, the Fc region is modified by substituting the serine residue at position 298 with an alanine residue (S298A).

In one embodiment, the Fc region is modified by substituting the leucine residue at position 328 with an arginine residue (L328R).

In one embodiment, the Fc region is modified by substituting the leucine residue at position 234 with an alanine residue and the leucine residue at position 235 with an alanine residue (L234A/L235A).

In one embodiment, the Fc region is modified by substituting the phenylalanine residue at position 234 with an alanine residue and the leucine residue at position 235 with an alanine residue (F234A/L235A).

In one embodiment, the Fc region is modified by substituting the glycine residue at position 236 with an arginine residue and the leucine residue at position 328 with an arginine residue (G236R/L328R).

It will be appreciated that any of the modifications listed above may be combined to decrease FcγR binding.

In one embodiment a TBM of the present disclosure comprises an Fc domain in which one or both Fc regions comprise one or more modifications that decrease Fc binding to FcγRIIIa without affecting the Fc's binding to FcγRII.

In one embodiment, the Fc region is modified by substituting the serine residue at position 239 with an alanine residue (S239A).

In one embodiment, the Fc region is modified by substituting the glutamic acid residue at position 269 with an alanine residue (E269A).

In one embodiment, the Fc region is modified by substituting the glutamic acid residue at position 293 with an alanine residue (E293A).

In one embodiment, the Fc region is modified by substituting the tyrosine residue at position 296 with a phenylalanine residue (Y296F).

In one embodiment, the Fc region is modified by substituting the valine residue at position 303 with an alanine residue (V303A).

In one embodiment, the Fc region is modified by substituting the alanine residue at position 327 with a glycine residue (A327G).

In one embodiment, the Fc region is modified by substituting the lysine residue at position 338 with an alanine residue (K338A).

In one embodiment, the Fc region is modified by substituting the aspartic acid residue at position 376 with an alanine residue (D376A).

It will be appreciated that any of the modifications listed above may be combined to decrease FcγRIIIa binding.

6.3.1.2. Fc Domains with Altered Complement Binding

The TBM of the disclosure may comprise an Fc domain in which one or both Fc regions comprises one or more modifications that alter Fc binding to complement. Altered complement binding may be increased binding or decreased binding.

In one embodiment the Fc region comprises one or more modifications which decrease its binding to C1q. Initiation of the classical complement pathway starts with binding of hexameric C1q protein to the CH2 domain of antigen bound IgG and IgM.

In one embodiment the TBM of the disclosure comprises an Fc domain in which one or both Fc regions comprises one or more modifications to decrease Fc binding to C1q.

In one embodiment, the Fc region is modified by substituting the leucine residue at position 234 with an alanine residue (L234A).

In one embodiment, the Fc region is modified by substituting the leucine residue at position 235 with an alanine residue (L235A).

In one embodiment, the Fc region is modified by substituting the leucine residue at position 235 with a glutamic acid residue (L235E).

In one embodiment, the Fc region is modified by substituting the glycine residue at position 237 with an alanine residue (G237A).

In one embodiment, the Fc region is modified by substituting the lysine residue at position 322 with an alanine residue (K322A).

In one embodiment, the Fc region is modified by substituting the proline residue at position 331 with an alanine residue (P331A).

In one embodiment, the Fc region is modified by substituting the proline residue at position 331 with a serine residue (P331S).

In one embodiment, a TBM of the disclosure comprises an Fc domain derived from IgG4. IgG4 has a naturally lower complement activation profile than IgG1, but also weaker binding of FcγR. Thus, in one embodiment, the TBM comprises an IgG4 Fc domain and also comprises one or more modifications that increase FcγR binding.

It will be appreciated that any of the modifications listed above may be combined to reduce C1q binding.

6.3.1.3. Fc Domains with Altered Disulfide Architecture

The TBM of the disclosure can include an Fc domain comprising one or more modifications to create and/or remove a cysteine residue. Cysteine residues have an important role in the spontaneous assembly of Fc-based multispecific binding molecules, by forming disulfide bridges between individual pairs of polypeptide monomers. Thus, by altering the number and/or position of cysteine residues, it is possible to modify the structure of the TBM to produce a protein with improved therapeutic properties.

A TBM of the present disclosure can comprise an Fc domain in which one or both Fc regions, preferably both Fc regions, comprise a cysteine residue at position 309. In one embodiment, the cysteine residue at position 309 is created by a modification, e.g., for an Fc domain derived from IgG1, the leucine residue at position 309 is substituted with a cysteine residue (L309C), for an Fc domain derived from IgG2, the valine residue at position 309 is substituted with a cysteine residue (V309C).

In one embodiment, the Fc region is modified by substituting the valine residue at position 308 with a cysteine residue (V308C).

In one embodiment, two disulfide bonds in the hinge region are removed by mutating a core hinge sequence CPPC (SEQ ID NO: 2) to SPPS (SEQ ID NO: 3).

6.3.1.4. Fc Domains with Altered Glycosylation

In certain aspects, TBMs with improved manufacturability are provided that comprise fewer glycosylation sites than a corresponding immunoglobulin. These proteins have less complex post translational glycosylation patterns and are thus simpler and less expensive to manufacture.

In one embodiment a glycosylation site in the CH2 domain is removed by substituting the asparagine residue at position 297 with an alanine residue (N297A) or a glutamine residue (N297Q). In addition to improved manufacturability, these aglycosyl mutants also reduce FcγR binding as described herein above.

6.3.1.5. Fc Heterodimerization

Many multispecific molecule formats entail dimerization between two Fc regions that, unlike a native immunoglobulin, are operably linked to non-identical antigen-binding domains (or portions thereof, e.g., a VH or VH-CH1 of a Fab). Inadequate heterodimerization of two Fc regions to form an Fc domain has always been an obstacle for increasing the yield of desired multispecific molecules and represents challenges for purification. A variety of approaches available in the art can be used in for enhancing dimerization of Fc regions that might be present in the TBMs of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. W02009/089004A1.

The present disclosure provides TBMs comprising Fc heterodimers, i.e., Fc domains comprising heterologous, non-identical Fc regions. Heterodimerization strategies are used to enhance dimerization of Fc regions operably linked to different ABMs (or portions thereof, e.g., a VH or VH-CH1 of a Fab) and reduce dimerization of Fc regions operably linked to the same ABM or portion thereof. Typically, each Fc region in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Typically, the TBMs comprise other antibody fragments in addition to CH3 domains, such as, CH1 domains, CH2 domains, hinge domain, VH domain(s), VL domain(s), CDR(s), and/or antigen-binding fragments described herein. In some embodiments, the two hetero-polypeptides are two heavy chains forming a bispecific or multispecific molecules. Heterodimerization of the two different heavy chains at CH3 domains give rise to the desired antibody or antibody-like molecule, while homodimerization of identical heavy chains will reduce yield of the desired antibody or molecule. In an exemplary embodiment, the two or more hetero-polypeptide chains comprise two chains comprising CH3 domains and forming the molecules of any of the multispecific molecule formats described above of the present disclosure. In an embodiment, the two hetero-polypeptide chains comprising CH3 domains comprise modifications that favor heterodimeric association of the polypeptides, relative to unmodified chains. Various examples of modification strategies are provided below in Table 2 and Sections 6.3.1.5.1 to 6.3.1.5.3.

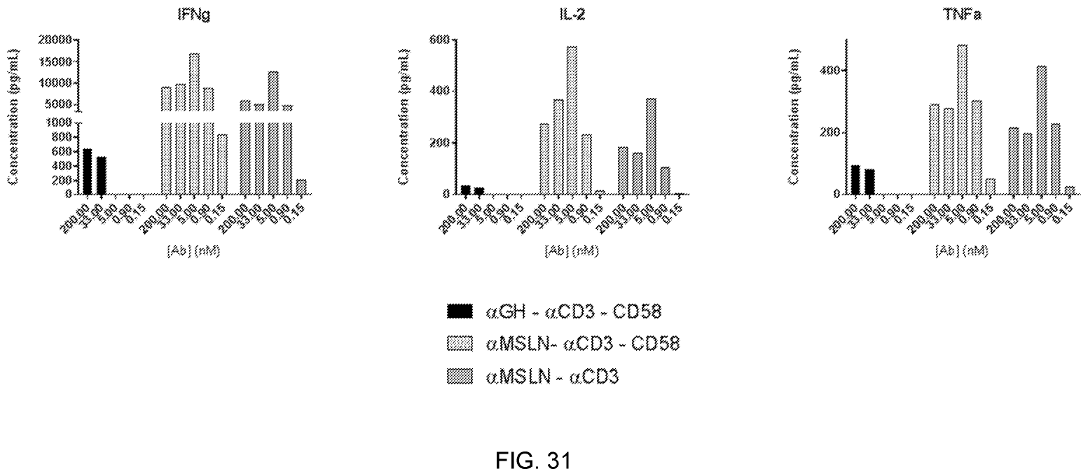

TABLE 2
Fc Heterodimerization Strategies

| NO. | STRATEGY | CH3 DOMAIN 1 | CH3 DOMAIN 2 | REFERENCES |
|---|---|---|---|---|
| Fc 1 | knobs-into-holes | T366Y | Y407T | Ridgway et al., 1996, |
| | (Y-T) | | | Protein Eng 9:617-21 |
| Fc 2 | knobs-into-holes | S354C, T366W | Y349C, T366S, | Atwell et al., 1997, J |
| | (CW-CSAV) | | L368A, Y407V | Mol Biol. 270(1):26- |
| | | | | 35; Merchant et al., |
| | | | | 1998, Nat Biotechnol |
| | | | | 16:677-681 |
| Fc 3 | HA-TF | S364H, F405A | Y349T, T394F | Moore et al., 2011, |
| | | | | MAbs 3(6):546-57 |
| Fc 4 | ZW1 (VYAV- | T350V, L351Y, | T350V, T366L, | Von Kreudenstein et |
| | VLLW) | F405A, Y407V | K392L, T394W | al., 2013, MAbs |
| | | | | 5:646-54 |
| Fc 5 | CH3 charge pairs | K392D, K409D | E356K, D399K | Gunasekaran et al., |
| | (DD-KK) | | | 2010, J Biol Chem |
| | | | | 285:19637-46 |
| Fc 6 | IgG1 hingE, CH3 | IgG1: D221E, | IgG1: D221R, | Strop et al., 2012, J |
| | charge pairs (EEE- | P228E, L368E | P228R, K409R | Mol Biol 420:204-19 |
| | RRR) | | | |
| Fc 7 | IgG2 hingE, CH3 | IgG2: C223E, | IgG2: C223R, | Strop et al., 2012, J |
| | charge pairs (EEE- | P228E, L368E | E225R, P228R, | Mol Biol 420:204-19 |
| | RRRR) | | K409R | |
| Fc 8 | EW-RVT | K360E, K409W, | Q347R, D399V, | Choi et al., 2013, Mol |
| | | | F405T | Cancer Ther |
| | | | | 12:2748-59 |
| Fc 9 | EW-RVTS-S | K360E, K409W, | Q347R, D399V, | Choi et al., 2015, Mol |
| | | Y349C | F405T, S354C | Immunol 65:377-83 |
| Fc 10 | Biclonic | 366K (+351K) | 351D or E or D at | Geuijen et al., 2014, |
| | | | 349, 368, 349, or | Journal of Clinical |
| | | | 349 + 355 | Oncology |
| | | | | 32:suppl:560 |
| Fc 11 | DuoBody (L-R) | F405L | K409R | Labrijn et al., 2013, |
| | | | | Proc Natl Acad Sci |
| | | | | U.S.A 110:5145-50 |
| Fc 12 | SEEDbody | IgG/A chimera | IgG/A chimera | Davis et al., 2010, |
| | | | | Protein Eng Des Sel |
| | | | | 23:195-202 |
| Fc 13 | BEAT | residues from | residues from | Moretti et al., 2013, |
| | | TCRα interface | TCRβ interface | BMC Proceedings |
| | | | | 7(Suppl 6):O9 |
| Fc 14 | 7.8.60 (DMA- | K360D, D399M, | E345R, Q347R, | Leaver-Fey et al., |
| | RRW) | Y407A | T366V, K409V | Structure 24:641-51 |
| Fc 15 | 20.8.34 (SYMV- | Y349S, K370Y, | E356G, E357D, | Leaver-Fey et al., |
| | GDQA) | T366M, K409V | S364Q, Y407A | Structure 24:641-51 |
| Fc 16 | Skew variant | None | none | FIG. 34 of U.S. |
| | 12757 | | | 2016/0355600 |
| Fc 17 | Skew variant | L368D, K370S | S364K | FIG. 34 of U.S. |
| | 12758 | | | 2016/0355600 |
| Fc 18 | Skew variant | L368D, K370S | S364K, E357L | FIG. 34 of U.S. |
| | 12759 | | | 2016/0355600 |
| Fc 19 | Skew variant | L368D, K370S | S364K, E357Q | FIG. 34 of U.S. |
| | 12760 | | | 2016/0355600 |
| Fc 20 | Skew variant | T411E, K360E, | D401K | FIG. 34 of U.S. |
| | 12761 | Q362E | | 2016/0355600 |
| Fc 21 | Skew variant | L368E, K370S | S364K | FIG. 34 of U.S. |
| | 12496 | | | 2016/0355600 |
| Fc 22 | Skew variant | K370S | S364K | FIG. 34 of U.S. |
| | 12511 | | | 2016/0355600 |
| Fc 23 | Skew variant | L368E, K370S | S364K, E357Q | FIG. 34 of U.S. |
| | 12840 | | | 2016/0355600 |
| Fc 24 | Skew variant | K370S | S364K, E357Q | FIG. 34 of U.S. |
| | 12841 | | | 2016/0355600 |
| Fc 25 | Skew variant | L368E, K370S | S364K | FIG. 34 of U.S. |
| | 12894 | | | 2016/0355600 |
| Fc 26 | Skew variant | K370S | S364K | FIG. 34 of U.S. |
| | 12895 | | | 2016/0355600 |
| Fc 27 | Skew variant | L368E, K370S | S364K, E357Q | FIG. 34 of U.S. |
| | 12896 | | | 2016/0355600 |
| Fc 28 | Skew variant | K370S | S364K, E357Q | FIG. 34 of U.S. |
| | 12901 | | | 2016/0355600 |
| Fc 29 | pl_ISO(−) | I199T, N203D, | | FIG. 31 of U.S. |
| | | K274Q, R355Q, | | 2016/0355600 |
| | | N384S, K392N, | | |
| | | V397M, Q419E, | | |
| | | DEL447 | | |
| Fc 30 | pl _(−)_Isosteric_A | N208D, Q295E, | | FIG. 31 of U.S. |
| | | N384D, Q418E, | | 2016/0355600 |
| | | N421D | | |
| Fc 31 | pl_(−)_isosteric_B | N208D, Q295E, | | FIG. 31 of U.S. |
| | | Q418E, N421D | | 2016/0355600 |
| Fc 32 | pl_ISO(+RR) | Q196K, I199T, | | FIG. 31 of U.S. |
| | | P217R, P228R, | | 2016/0355600 |
| | | N276K | | |
| Fc 33 | pl_ISO(+) | Q196K, I199T, | | FIG. 31 of U.S. |
| | | N276K | | 2016/0355600 |
| Fc 34 | pl_(+)_isosteric_A | E269Q, E272Q, | | FIG. 31 of U.S. |
| | | E283Q, E357Q, | | 2016/0355600 |
| Fc 35 | pl_(+)_isosteric_B | E269Q, E272Q, | | FIG. 31 of U.S. |
| | | E283Q | | 2016/0355600 |
| Fc 36 | pl_(+) | E269Q, E272Q | | FIG. 31 of U.S. |
| | isosteric_E269Q, | | | 2016/0355600 |
| | E272Q | | | |
| Fc 37 | pl_(+)_isosteric_E | E269Q, E283Q | | FIG. 31 of U.S. |
| | 269Q, E283Q | | | 2016/0355600 |
| Fc 38 | pl_(+) | E272Q, E283Q | | FIG. 31 of U.S. |
| | isosteric_E2720, | | | 2016/0355600 |
| | E283Q | | | |
| Fc 39 | pl_(+)_isosteric_E | E269Q | | FIG. 31 of U.S. |
| | 269Q | | | 2016/0355600 |
| Fc 40 | Heterodimerization | F405A | T394F | FIG. 30A of U.S. |
| | | | | 2016/0355600 |
| Fc 41 | Heterodimerization | S364D | Y349K | FIG. 30A of U.S. |
| | | | | 2016/0355600 |
| Fc 42 | Heterodimerization | S364E | L368K | FIG. 30A of U.S. |
| | | | | 2016/0355600 |
| Fc 43 | Heterodimerization | S364E | Y349K | FIG. 30A of U.S. |
| | | | | 2016/0355600 |
| Fc 44 | Heterodimerization | S364F | K370G | FIG. 30A of U.S. |
| | | | | 2016/0355600 |
| Fc 45 | Heterodimerization | S364H | Y349K | FIG. 30A of U.S. |
| | | | | 2016/0355600 |
| Fc 46 | Heterodimerization | S364H | Y349T | FIG. 30A of U.S. |
| | | | | 2016/0355600 |
| Fc 47 | Heterodimerization | S364Y | K370G | FIG. 30A of U.S. |
| | | | | 2016/0355600 |
| Fc 48 | Heterodimerization | T411K | K370E | FIG. 30A of U.S. |
| | | | | 2016/0355600 |
| Fc 49 | Heterodimerization | V397S, F405A | T394F | FIG. 30A of U.S. |
| | | | | 2016/0355600 |
| Fc 50 | Heterodimerization | K370R, T411K | K370E, T411E | FIG. 30A of U.S. |
| | | | | 2016/0355600 |
| Fc 51 | Heterodimerization | L351E, S364D | Y349K, L351K | FIG. 30A of U.S. |
| | | | | 2016/0355600 |
| Fc 52 | Heterodimerization | L351E, S364E | Y349K, L351K | FIG. 30A of U.S. |
| | | | | 2016/0355600 |
| Fc 53 | Heterodimerization | L351E, T366D | L351K, T366K | FIG. 30A of U.S. |
| | | | | 2016/0355600 |
| Fc 54 | Heterodimerization | P395T, V397S, | T394F | FIG. 30A of U.S. |
| | | F405A | | 2016/0355600 |
| Fc 55 | Heterodimerization | S364D, K370G | S364Y, K370R | FIG. 30A of U.S. |
| | | | | 2016/0355600 |
| Fc 56 | Heterodimerization | S364D, T394F | Y349K, F405A | FIG. 30A of U.S. |
| | | | | 2016/0355600 |
| Fc 57 | Heterodimerization | S364E, F405A | Y349K, T394F | FIG. 30A of U.S. |
| | | | | 2016/0355600 |
| Fc 58 | Heterodimerization | S364E, F405S | Y349K, T394Y | FIG. 30A of U.S. |
| | | | | 2016/0355600 |
| Fc 59 | Heterodimerization | S364E, T411E | Y349K, D401K | FIG. 30A of U.S. |
| | | | | 2016/0355600 |
| Fc 60 | Heterodimerization | S364H, D401K | Y349T, T411E | FIG. 30A of U.S. |
| | | | | 2016/0355600 |
| Fc 61 | Heterodimerization | S364H, F405A | Y349T, T394F | FIG. 30A of U.S. |
| | | | | 2016/0355600 |
| Fc 62 | Heterodimerization | S364H, T394F | Y349T, F405A | FIG. 30A of U.S. |
| | | | | 2016/0355600 |
| Fc 63 | Heterodimerization | Y349C, S364E | Y349K, S354C | FIG. 30A of U.S. |
| | | | | 2016/0355600 |
| Fc 64 | Heterodimerization | L351E, S364D, | Y349K, L351K, | FIG. 30A of U.S. |
| | | F405A | T394F | 2016/0355600 |
| Fc 65 | Heterodimerization | L351K, S364H, | Y349T, L351E, | FIG. 30A of U.S. |
| | | D401K | T411E | 2016/0355600 |
| Fc 66 | Heterodimerization | S364E, T411E, | Y349K, T394F, | FIG. 30A of U.S. |
| | | F405A | D401K | 2016/0355600 |
| Fc 67 | Heterodimerization | S364H, D401K, | Y349T, T394F, | FIG. 30A of U.S. |
| | | F405A | T411E | 2016/0355600 |
| Fc 68 | Heterodimerization | S364H, F405A, | Y349T, T394F, | FIG. 30A of U.S. |
| | | T411E | D401K | 2016/0355600 |
| Fc 69 | Heterodimerization | T411E, K360E, | D401K | FIG. 30C of U.S. |
| | | N390D | | 2016/0355600 |
| Fc 70 | Heterodimerization | T411E, Q362E, | D401K | FIG. 30C of U.S. |
| | | N390D | | 2016/0355600 |
| Fc 71 | Heterodimerization | T411E, Q347R | D401K, K360D | FIG. 30C of U.S. |
| | | | | 2016/0355600 |
| Fc 72 | Heterodimerization | T411E, Q347R | D401K, K360E | FIG. 30C of U.S. |
| | | | | 2016/0355600 |
| Fc 73 | Heterodimerization | T411E, K360 | D401K, Q347K | FIG. 30C of U.S. |
| | | | | 2016/0355600 |
| Fc 74 | Heterodimerization | T411E, K360D | D401K, Q347R | FIG. 30C of U.S. |
| | | | | 2016/0355600 |
| Fc 75 | Heterodimerization | T411E, K360E | D401K, Q347K | FIG. 30C of U.S. |
| | | | | 2016/0355600 |
| Fc 76 | Heterodimerization | T411E, K360E | D401K, Q347R | FIG. 30C of U.S. |
| | | | | 2016/0355600 |
| Fc 77 | Heterodimerization | T411E, S364K | D401K, K370S | FIG. 30C of U.S. |
| | | | | 2016/0355600 |
| Fc 78 | Heterodimerization | T411E, K370S | D401K, S364K | FIG. 30C of U.S. |
| | | | | 2016/0355600 |
| Fc 79 | Heterodimerization | Q347E | E357Q | FIG. 30C of U.S. |
| | | | | 2016/0355600 |
| Fc 80 | Heterodimerization | Q347E | E357Q, Q362K | FIG. 30C of U.S. |
| | | | | 2016/0355600 |
| Fc 81 | Heterodimerization | K360D, Q362E | Q347R | FIG. 30C of U.S. |
| | | | | 2016/0355600 |
| Fc 82 | Heterodimerization | K360D, Q362E | D401K | FIG. 30C of U.S. |
| | | | | 2016/0355600 |
| Fc 83 | Heterodimerization | K360D, Q362E | Q347R, D401K | FIG. 30C of U.S. |
| | | | | 2016/0355600 |
| Fc 84 | Heterodimerization | K360E, Q362E | Q347R | FIG. 30C of U.S. |
| | | | | 2016/0355600 |
| Fc 85 | Heterodimerization | K360E, Q362E | D401K | FIG. 30C of U.S. |
| | | | | 2016/0355600 |
| Fc 86 | Heterodimerization | K360E, Q362E | Q347R, D401K | FIG. 30C of U.S. |
| | | | | 2016/0355600 |
| Fc 87 | Heterodimerization | Q362E, N39QD | D401K | FIG. 30C of U.S. |
| | | | | 2016/0355600 |
| Fc 88 | Heterodimerization | Q347E, K360D | D401N | FIG. 30C of U.S. |
| | | | | 2016/0355600 |
| Fc 89 | Heterodimerization | K360D | Q347R, N390K | FIG. 30C of U.S. |
| | | | | 2016/0355600 |
| Fc 90 | Heterodimerization | K360D | N390K, D401N | FIG. 30C of U.S. |
| | | | | 2016/0355600 |
| Fc 91 | Heterodimerization | K360E | Y349H | FIG. 30C of U.S. |
| | | | | 2016/0355600 |
| Fc 92 | Heterodimerization | K370S, Q347E | S364K | FIG. 30C of U.S. |
| | | | | 2016/0355600 |
| Fc 93 | Heterodimerization | K370S, E357L | S364K | FIG. 30C of U.S. |
| | | | | 2016/0355600 |
| Fc 94 | Heterodimerization | K370S, E357Q | S364K | FIG. 30C of U.S. |
| | | | | 2016/0355600 |
| Fc 95 | Heterodimerization | K370S, Q347E, | S364K | FIG. 30C of U.S. |
| | | E357L | | 2016/0355600 |
| Fc 96 | Heterodimerization | K370S, Q347E, | S364K | FIG. 30C of U.S. |
| | | E357Q | | 2016/0355600 |
| Fc 97 | Heterodimerization | L368D, K370S, | S364K | FIG. 30D of U.S. |
| | | Q347E | | 2016/0355600 |
| Fc 98 | Heterodimerization | L368D, K370S, | S364K | FIG. 30D of U.S. |
| | | E357L | | 2016/0355600 |
| Fc 99 | Heterodimerization | L368D, K370S, | S364K | FIG. 30D of U.S. |
| | | E357Q | | 2016/0355600 |
| Fc 100 | Heterodimerization | L368D, K370S, | S364K | FIG. 30D of U.S. |
| | | Q347E, E357L | | 2016/0355600 |
| Fc 101 | Heterodimerization | L368D, K370S, | S364K | FIG. 30D of U.S. |
| | | Q347E, E357Q | | 2016/0355600 |
| Fc 102 | Heterodimerization | L368E, K370S, | S364K | FIG. 30D of U.S. |
| | | Q347E | | 2016/0355600 |
| Fc 103 | Heterodimerization | L368E, K370S, | S364K | FIG. 30D of U.S. |
| | | E357L | | 2016/0355600 |
| Fc 104 | Heterodimerization | L368E, K370S, | S364K | FIG. 30D of U.S. |
| | | E357Q | | 2016/0355600 |
| Fc 105 | Heterodimerization | L368E, K370S, | S364K | FIG. 30D of U.S. |
| | | Q347E, E357L | | 2016/0355600 |
| Fc 106 | Heterodimerization | L368E, K370S, | S364K | FIG. 30D of U.S. |
| | | Q347E, E357Q | | 2016/0355600 |
| Fc 107 | Heterodimerization | L368D, K370T, | S364K | FIG. 30D of U.S. |
| | | Q347E | | 2016/0355600 |
| Fc 108 | Heterodimerization | L368D, K370T, | S364K | FIG. 30D of U.S. |
| | | E357L | | 2016/0355600 |
| Fc 109 | Heterodimerization | L368D, K370T, | S364K | FIG. 30D of U.S. |
| | | E357Q | | 2016/0355600 |
| Fc 110 | Heterodimerization | L368D, K370T, | S364K | FIG. 30D of U.S. |
| | | Q347E, E357L | | 2016/0355600 |
| Fc 111 | Heterodimerization | L368D, K370T, | S364K | FIG. 30D of U.S. |
| | | Q347E, E357Q | | 2016/0355600 |
| Fc 112 | Heterodimerization | L368E, K370T, | S364K | FIG. 30D of U.S. |
| | | Q347E | | 2016/0355600 |
| Fc 113 | Heterodimerization | L368E, K370T, | S364K | FIG. 30D of U.S. |
| | | E357L | | 2016/0355600 |
| Fc 114 | Heterodimerization | L368E, K370T, | S364K | FIG. 30D of U.S. |
| | | E357Q | | 2016/0355600 |
| Fc 115 | Heterodimerization | L368E, K370T, | S364K | FIG. 30D of U.S. |
| | | Q347E, E357L | | 2016/0355600 |
| Fc 116 | Heterodimerization | L368E, K370T, | S364K | FIG. 30D of U.S. |
| | | Q347E, E357Q | | 2016/0355600 |
| Fc 117 | Heterodimerization | T411E, Q362E | D401K, T411K | FIG. 30D of U.S. |
| | | | | 2016/0355600 |
| Fc 118 | Heterodimerization | T411E, N390D | D401K, T411K | FIG. 30D of U.S. |
| | | | | 2016/0355600 |
| Fc 119 | Heterodimerization | T411E, Q362E | D401R, T411R | FIG. 30D of U.S. |
| | | | | 2016/0355600 |
| Fc 120 | Heterodimerization | T411E, N390D | D401R, T411R | FIG. 30D of U.S. |
| | | | | 2016/0355600 |
| Fc 121 | Heterodimerization | Y407T | T366Y | FIG. 30D of U.S. |
| | | | | 2016/0355600 |
| Fc 122 | Heterodimerization | F405A | T394W | FIG. 30D of U.S. |
| | | | | 2016/0355600 |
| Fc 123 | Heterodimerization | T366Y, F405A | T394W, Y407T | FIG. 30D of U.S. |
| | | | | 2016/0355600 |
| Fc 124 | Heterodimerization | T3665, L368A, | T366W | FIG. 30D of U.S. |
| | | Y407V | | 2016/0355600 |
| Fc 125 | Heterodimerization | T366S, L368A, | T366W, S354C | FIG. 30D of U.S. |
| | | Y407V, Y349C | | 2016/0355600 |
| Fc 126 | Heterodimerization | K392D, K4Q9D | E356K, D399K | FIG. 30E of U.S. |
| | | | | 2016/0355600 |
| Fc 127 | Heterodimerization | K370D, K392D, | E356K, E357K, | FIG. 30E of U.S. |
| | | K409D | D399K | 2016/0355600 |
| Fc 128 | Heterodimerization | I199T, N203D, | Q196K, L99T, | FIG. 30E of U.S. |
| | | K247Q, R355Q, | P217R, P228R, | 2016/0355600 |
| | | N384S, K392N, | N276K | |
| | | V397M, Q419E, | | |
| | | K447 | | |
| Fc 129 | Heterodimerization | I199T, N203D, | Q196K, L99T, | FIG. 30E of U.S. |
| | | K247Q, R355Q, | N276K | 2016/0355600 |
| | | N384S, K392N, | | |
| | | V397M, Q419E, | | |
| | | K447 | | |
| Fc 130 | Heterodimerization | N384S, K392N, | N276K | FIG. 30E of U.S. |
| | | V397M, Q419E | | 2016/0355600 |
| Fc 131 | Heterodimerization | D221E, P228E, | D221R, P228R, | FIG. 30E of U.S. |
| | | L368E | K409R | 2016/0355600 |
| Fc 132 | Heterodimerization | C220E, P228E, | C220R, E224R, | FIG. 30E of U.S. |
| | | L368E | P228R, K409R | 2016/0355600 |
| Fc 133 | Heterodimerization | F405L | K409R | FIG. 30E of U.S. |
| | | | | 2016/0355600 |
| Fc 134 | Heterodimerization | T366I, K392M, | F405A, Y407V | FIG. 30E of U.S. |
| | | T394W | | 2016/0355600 |
| Fc 135 | Heterodimerization | T366V, K409F | L351Y, Y407A | FIG. 30E of U.S. |
| | | | | 2016/0355600 |
| Fc 136 | Heterodimerization | T366A, K392E, | D399R, S400R, | FIG. 30E of U.S. |
| | | K409F, T411E | Y407A | 2016/0355600 |
| Fc 137 | Heterodimerization | L351K | L351E | FIG. 30E of U.S. |
| | | | | 2016/0355600 |
| Fc 138 | Heterodimerization | I199T, N203D, | Q196K, L199T, | FIG. 30E of U.S. |
| | | K247Q, R355Q, | P217R, P228R, | 2016/0355600 |
| | | Q419E, K447 | N276K | |
| Fc 139 | Heterodimerization | I199T, | Q196K, I199T, | FIG. 30E of U.S. |
| | | N203D ,K247Q, | N276K | 2016/0355600 |
| | | R355Q, Q419E, | | |
| | | K447 | | |
| Fc 140 | Heterodimerization | I199T, N203D, | | FIG. 30E of U.S. |
| | | K274Q, R355Q, | | 2016/0355600 |
| | | N384S, K392N, | | |
| | | V397M, Q419E | | |
| | | DEL447 | | |
| Fc 141 | Heterodimerization | N208D, Q295E | | FIG. 30E of U.S. |
| | | N384D, Q418E | | 2016/0355600 |
| | | N421D | | |
| Fc 142 | Heterodimerization | N208D, Q295E | | FIG. 30E of U.S. |
| | | Q418E, N421D | | 2016/0355600 |
| Fc 143 | Heterodimerization | Q196K, I199T | | FIG. 30E of U.S. |
| | | P217R, P228R | | 2016/0355600 |
| | | N276K | | |
| Fc 144 | Heterodimerization | Q196K, I199T | | FIG. 30E of U.S. |
| | | N276K | | 2016/0355600 |
| Fc 145 | Heterodimerization | E269Q, E272Q | | FIG. 30E of U.S. |
| | | E283Q, E357Q | | 2016/0355600 |
| Fc 146 | Heterodimerization | E269Q, E272Q | | FIG. 30E of U.S. |
| | | E283Q, | | 2016/0355600 |
| Fc 147 | Heterodimerization | E269Q, E272Q | | FIG. 30E of U.S. |
| | | | | 2016/0355600 |
| Fc 148 | Heterodimerization | E269Q, E283Q | | FIG. 30E of U.S. |
| | | | | 2016/0355600 |
| Fc 149 | Heterodimerization | E272Q, E283Q | | FIG. 30E of U.S. |
| | | | | 2016/0355600 |
| Fc 150 | Heterodimerization | E269Q | | FIG. 30E of U.S. |
| | | | | z2016/0355600 |

6.3.1.5.1. Knob-in-Hole (KIH)

TBMs of the disclosure may comprise one or more, e.g., a plurality, of modifications to one or more of the constant domains of an Fc domain, e.g., to the CH3 domains. In one example, a TBM of the present disclosure comprises two polypeptides that each comprise a heavy chain constant domain of an antibody, e.g., a CH2 or CH3 domain. In an example, the two heavy chain constant domains, e.g., the CH2 or CH3 domains of the TBM comprise one or more modifications that allow for a heterodimeric association between the two chains. In one aspect, the one or more modifications are disposed on CH2 domains of the two heavy chains. In one aspect, the one or more modifications are disposed on CH3 domains of at least two polypeptides of the TBM. In one aspect, the one or more modifications to a first polypeptide of the TBM comprising a heavy chain constant domain can create a “knob” and the one or more modifications to a second polypeptide of the TBM creates a “hole,” such that heterodimerization of the polypeptide of the TBM comprising a heavy chain constant domain causes the “knob” to interface (e.g., interact, e.g., a CH2 domain of a first polypeptide interacting with a CH2 domain of a second polypeptide, or a CH3 domain of a first polypeptide interacting with a CH3 domain of a second polypeptide) with the “hole.” As the term is used herein, a “knob” refers to at least one amino acid side chain which projects from the interface of a first polypeptide of the TBM comprising a heavy chain constant domain and is therefore positionable in a compensatory “hole” in the interface with a second polypeptide of the TBM comprising a heavy chain constant domain so as to stabilize the heteromultimer, and thereby favor heteromultimer formation over homomultimer formation, for example. The knob may exist in the original interface or may be introduced synthetically (e.g. by altering nucleic acid encoding the interface). The preferred import residues for the formation of a knob are generally naturally occurring amino acid residues and are preferably selected from arginine (R), phenylalanine (F), tyrosine (Y) and tryptophan (W). Most preferred are tryptophan and tyrosine. In the preferred embodiment, the original residue for the formation of the protuberance has a small side chain volume, such as alanine, asparagine, aspartic acid, glycine, serine, threonine or valine.

A “hole” refers to at least one amino acid side chain which is recessed from the interface of a second polypeptide of the TBM comprising a heavy chain constant domain and therefore accommodates a corresponding knob on the adjacent interfacing surface of a first polypeptide of the TBM comprising a heavy chain constant domain. The hole may exist in the original interface or may be introduced synthetically (e.g. by altering nucleic acid encoding the interface). The preferred import residues for the formation of a hole are usually naturally occurring amino acid residues and are preferably selected from alanine (A), serine (S), threonine (T) and valine (V). Most preferred are serine, alanine or threonine. In the preferred embodiment, the original residue for the formation of the hole has a large side chain volume, such as tyrosine, arginine, phenylalanine or tryptophan.

In a preferred embodiment, a first CH3 domain is modified at residue 366, 405 or 407 to create either a “knob” or a hole” (as described above), and the second CH3 domain that heterodimerizes with the first CH3 domain is modified at: residue 407 if residue 366 is modified in the first CH3 domain, residue 394 if residue 405 is modified in the first CH3 domain, or residue 366 if residue 407 is modified in the first CH3 domain to create a “hole” or “knob” complementary to the “knob” or “hole” of the first CH3 domain.

In another preferred embodiment, a first CH3 domain is modified at residue 366, and the second CH3 domain that heterodimerizes with the first CH3 domain is modified at residues 366, 368 and/or 407, to create a “hole” or “knob” complementary to the “knob” or “hole” of the first CH3 domain. In one embodiment, the modification to the first CH3 domain introduces a tyrosine (Y) residue at position 366. In an embodiment, the modification to the first CH3 is T366Y. In one embodiment, the modification to the first CH3 domain introduces a tryptophan (W) residue at position 366. In an embodiment, the modification to the first CH3 is T366W. In some embodiments, the modification to the second CH3 domain that heterodimerizes with the first CH3 domain modified at position 366 (e.g., has a tyrosine (Y) or tryptophan (W) introduced at position 366, e.g., comprises the modification T366Y or T366W), comprises a modification at position 366, a modification at position 368 and a modification at position 407. In some embodiments, the modification at position 366 introduces a serine (S) residue, the modification at position 368 introduces an alanine (A), and the modification at position 407 introduces a valine (V). In some embodiments, the modifications comprise T366S, L368A and Y407V. In one embodiment the first CH3 domain of the multispecific molecule comprises the modification T366Y, and the second CH3 domain that heterodimerizes with the first CH3 domain comprises the modifications T366S, L368A and Y407V, or vice versa. In one embodiment the first CH3 domain of the multispecific molecule comprises the modification T366W, and the second CH3 domain that heterodimerizes with the first CH3 domain comprises the modifications T366S, L368A and Y407V, or vice versa.

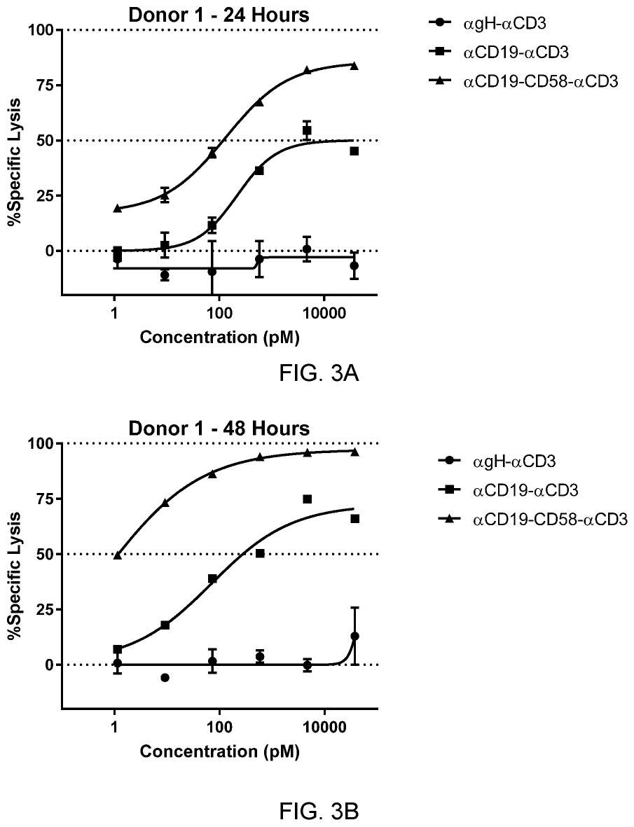

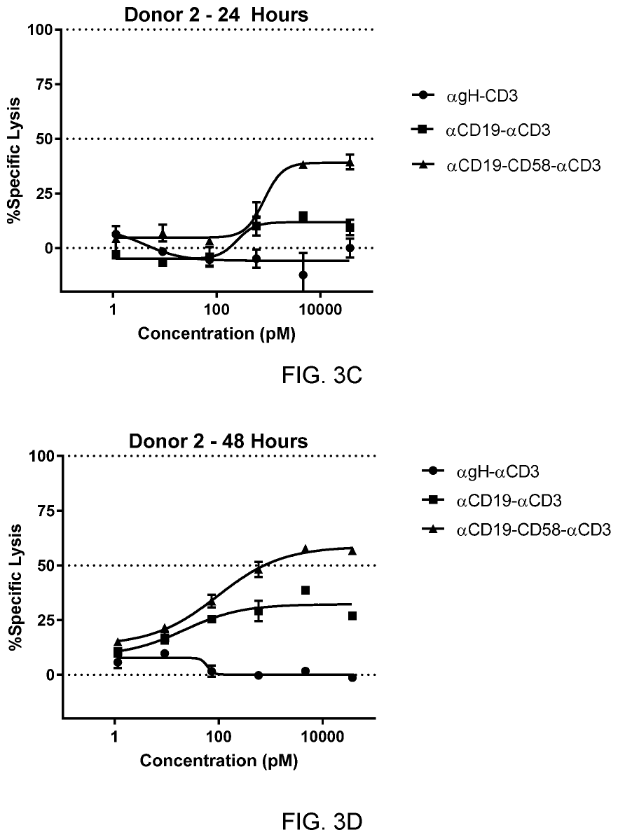

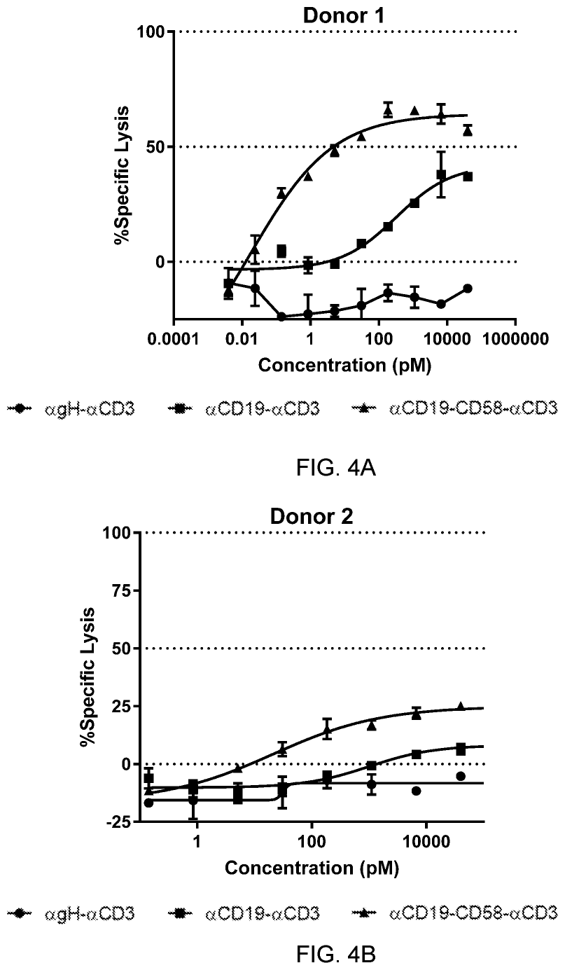

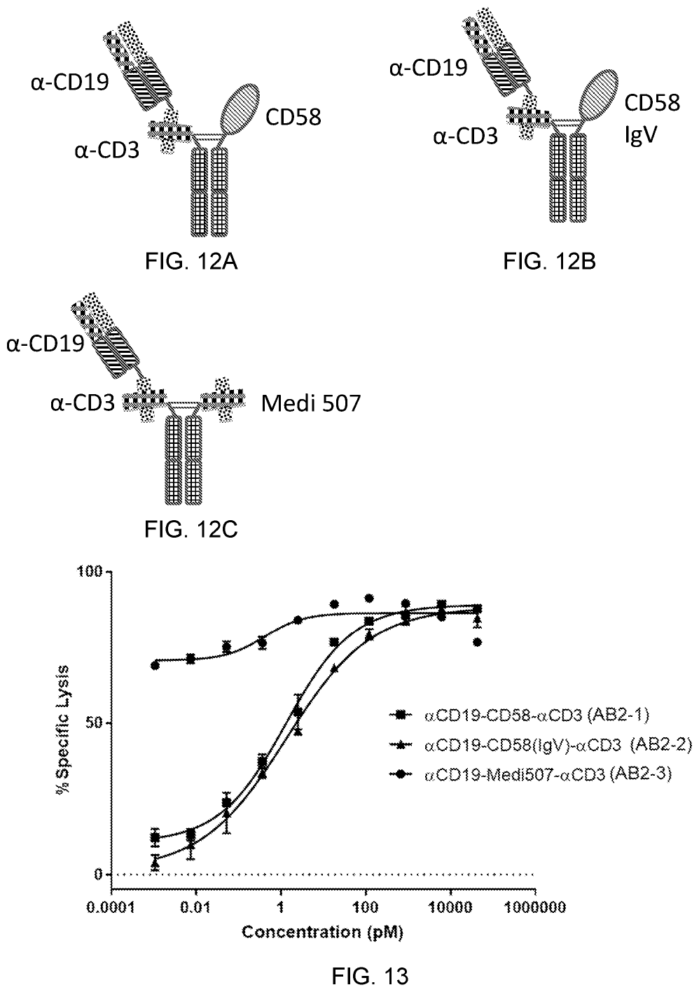

Additional steric or “skew” (e.g., knob in hole) modifications are described in PCT publication no. WO2014/145806 (for example, FIG. 3, FIG. 4 and FIG. 12 of WO2014/145806), PCT publication no. WO2014/110601, and PCT publication no. WO 2016/086186, WO 2016/086189, WO 2016/086196 and WO 2016/182751 the contents of which are incorporated herein in their entireties. An example of a KIH variant comprises a first constant chain comprising a L368D and a K370S modification, paired with a second constant chain comprising a S364K and E357Q modification.

Additional knob in hole modification pairs suitable for use in any of the multispecific molecules of the present disclosure are further described in, for example, WO1996/027011, and Merchant et al., 1998, Nat. Biotechnol., 16:677-681, the contents of which are hereby incorporated by reference in their entirety.

In further embodiments, the CH3 domains may be additionally modified to introduce a pair of cysteine residues. Without being bound by theory, it is believed that the introduction of a pair of cysteine residues capable of forming a disulfide bond provide stability to heterodimerized TBMs comprising paired CH3 domains. In some embodiments, the first CH3 domain comprises a cysteine at position 354, and the second CH3 domain that heterodimerizes with the first CH3 domain comprises a cysteine at position 349. In some embodiments, the first CH3 domain comprises a cysteine at position 354 (e.g., comprises the modification S354C) and a tyrosine (Y) at position 366 (e.g., comprises the modification T366Y), and the second CH3 domain that heterodimerizes with the first CH3 domain comprises a cysteine at position 349 (e.g., comprises the modification Y349C), a serine at position 366 (e.g., comprises the modification T366S), an alanine at position 368 (e.g., comprises the modification L368A), and a valine at position 407 (e.g., comprises the modification Y407V). In some embodiments, the first CH3 domain comprises a cysteine at position 354 (e.g., comprises the modification S354C) and a tryptophan (W) at position 366 (e.g., comprises the modification T366W), and the second CH3 domain that heterodimerizes with the first CH3 domain comprises a cysteine at position 349 (e.g., comprises the modification Y349C), a serine at position 366 (e.g., comprises the modification T366S), an alanine at position 368 (e.g., comprises the modification L368A), and a valine at position 407 (e.g., comprises the modification Y407V).

6.3.1.5.2. Alternative Knob and Hole: IgG Heterodimerization

Heterodimerization of polypeptide chains of a TBM comprising paired CH3 domains can be increased by introducing one or more modifications in a CH3 domain which is derived from the IgG1 antibody class. In an embodiment, the modifications comprise a K409R modification to one CH3 domain paired with F405L modification in the second CH3 domain. Additional modifications may also, or alternatively, be at positions 366, 368, 370, 399, 405, 407, and 409. Preferably, heterodimerization of polypeptides comprising such modifications is achieved under reducing conditions, e.g., 10-100 mM 2-MEA (e.g., 25, 50, or 100 mM 2-MEA) for 1-10, e.g., 1.5-5, e.g., 5, hours at 25-37 C, e.g., 25 C or 37 C.

The amino acid replacements described herein can be introduced into the CH3 domains using techniques which are well known in the art (see, e.g., McPherson, ed., 1991, Directed Mutagenesis: a Practical Approach; Adelman et al., 1983, DNA, 2:183).

The IgG heterodimerization strategy is further described in, for example, WO2008/119353, WO2011/131746, and WO2013/060867, the contents of which are hereby incorporated by reference in their entirety.

In any of the embodiments described in this Section, the CH3 domains can be additionally modified to introduce a pair of cysteine residues as described in Section 6.3.1.5.1.

6.3.1.5.3. Polar Bridge

Heterodimerization of polypeptide chains of TBMs comprising an Fc domain can be increased by introducing modifications based on the “polar-bridging” rationale, which is to make residues at the binding interface of the two polypeptide chains to interact with residues of similar (or complimentary) physical property in the heterodimer configuration, while with residues of different physical property in the homodimer configuration. In particular, these modifications are designed so that, in the heterodimer formation, polar residues interact with polar residues, while hydrophobic residues interact with hydrophobic residues. In contrast, in the homodimer formation, residues are modified so that polar residues interact with hydrophobic residues. The favorable interactions in the heterodimer configuration and the unfavorable interactions in the homodimer configuration work together to make it more likely for Fc regions to form heterodimers than to form homodimers.

In an exemplary embodiment, the above modifications are generated at one or more positions of residues 364, 368, 399, 405, 409, and 411 of a CH3 domain.

In some embodiments, one or more modifications selected from S364L, T366V, L368Q, N399K, F405S, K409F and R411K are introduced into one of the two CH3 domains. One or more modifications selected from Y407F, K409Q and T411N can be introduced into the second CH3 domain.

In another embodiment, one or more modifications selected from a group consisting of S364L, T366V, L368Q, D399K, F405S, K409F and T411K are introduced into one CH3 domain, while one or more modifications selected from Y407F, K409Q and T411D are introduced into the second CH3 domain.

In one exemplary embodiment, the original residue of threonine at position 366 of one CH3 domain is replaced by valine, while the original residue of tyrosine at position 407 of the other CH3 domain is replaced by phenylalanine.

In another exemplary embodiment, the original residue of serine at position 364 of one CH3 domain is replaced by leucine, while the original residue of leucine at position 368 of the same CH3 domain is replaced by glutamine.

In yet another exemplary embodiment, the original residue of phenylalanine at position 405 of one CH3 domain is replaced by serine and the original residue of lysine at position 409 of this CH3 domain is replaced by phenylalanine, while the original residue of lysine at position 409 of the other CH3 domain is replaced by glutamine.

In yet another exemplary embodiment, the original residue of aspartic acid at position 399 of one CH3 domain is replaced by lysine, and the original residue of threonine at position 411 of the same CH3 domain is replaced by lysine, while the original residue of threonine at position 411 of the other CH3 domain is replaced by aspartic acid.

The amino acid replacements described herein can be introduced into the CH3 domains using techniques which are well known in the art (see, e.g., McPherson, ed., 1991, Directed Mutagenesis: a Practical Approach; Adelman et al., 1983, DNA, 2:183). The polar bridge strategy is described in, for example, WO2006/106905, WO2009/089004 and K. Gunasekaran, et al. (2010) The Journal of Biological Chemistry, 285:19637-19646, the contents of which are hereby incorporated by reference in their entirety.

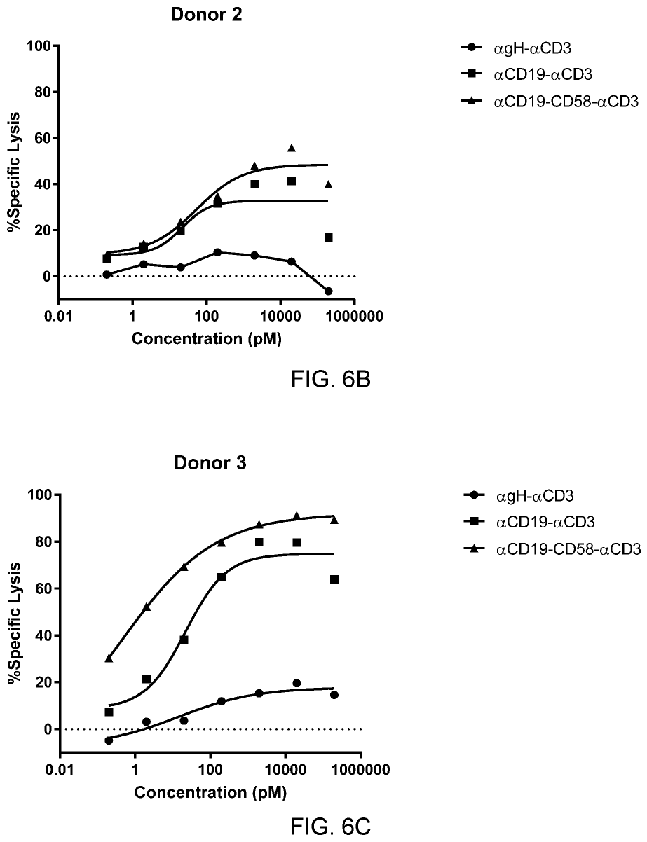

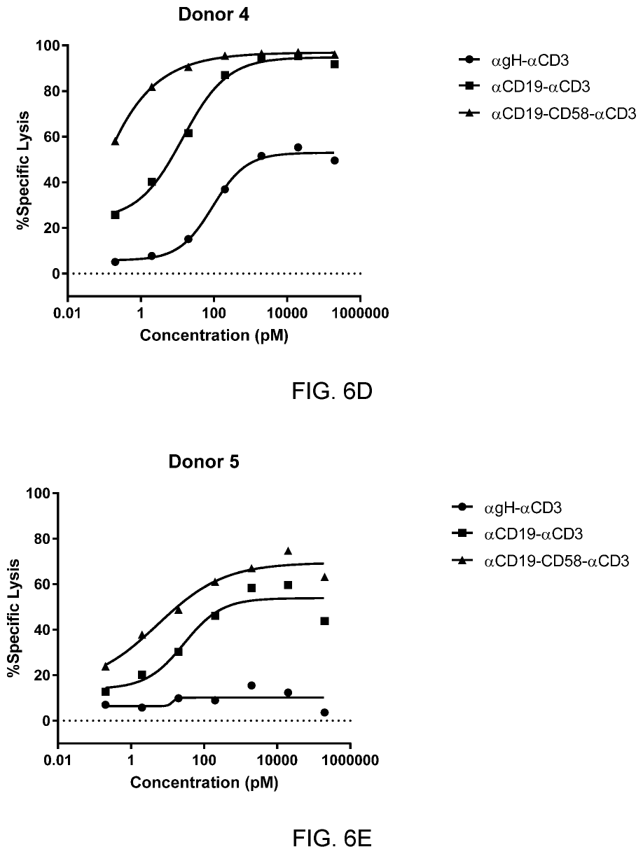

Additional polar bridge modifications are described in, for example, PCT publication no. WO2014/145806 (for example, FIG. 6 of WO2014/145806), PCT publication no. WO2014/110601, and PCT publication no. WO 2016/086186, WO 2016/086189, WO 2016/086196 and WO 2016/182751 the contents of which are incorporated herein in their entireties. An example of a polar bridge variant comprises a constant chain comprising a N208D, Q295E, N384D, Q418E and N421D modification.

In any of the embodiments described herein, the CH3 domains may be additionally modified to introduce a pair of cysteine residues as described in Section 6.3.1.5.1.

Additional strategies for enhancing heterodimerization are described in, for example, WO2016/105450, WO2016/086186, WO2016/086189, WO2016/086196, WO2016/141378, and WO2014/145806, and WO2014/110601, the entire contents of each of which is hereby incorporated by reference in its entirety. Any of said strategies can be employed in a TBM described herein.

6.3.2. Hinge Regions

The TBMs of the disclosure can also comprise hinge regions, e.g., connecting an antigen-binding module to an Fc region. The hinge region can be a native or a modified hinge region. Hinge regions are typically found at the N-termini of Fc regions.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO9915549, WO2005003170, WO2005003169, WO2005003170, WO9825971 and WO2005003171 and these are incorporated herein by reference.

Examples of suitable hinge sequences are shown in Table 3.

TABLE 3
Hinge Sequences

| Hinge | Hinge | | SEQ ID |
|---|---|---|---|
| Name | Description | Hinge Sequence | NO: |
| H1 | Human IgA1 | VPSTPPTPSPSTPPTPSPS | 4 |
| H2 | Human IgA2 | VPPPPP | 5 |
| H3 | Human IgD | ESPKAQASSVPTAQPQAEGS | 6 |
| | | LAKATTAPATTRNTGRGGEE | |
| | | KKKEKEKEEQEERETKTP | |
| H4 | Human IgG1 | EPKSCDKTHTCPPCP | 7 |
| H5 | Human IgG2 | ERKCCVECPPCP | 8 |
| H6 | Human IgG3 | ELKTPLGDTTHTCPRCPEPK | 9 |
| | | SCDTPPPCPRCPEPKSCDTP | |
| | | PPCPRCPEPKSCDTPPPCPR | |
| | | CP | |
| H7 | Human IgG4 | ESKYGPPCPSCP | 10 |
| H8 | Human IgG4(P) | ESKYGPPCPPCP | 11 |
| H9 | Engineered v1 | CPPC | 2 |
| H10 | Engineered v2 | CPSC | 12 |
| H11 | Engineered v3 | CPRC | 13 |
| H12 | Engineered v4 | SPPC | 14 |
| H13 | Engineered v5 | CPPS | 15 |
| H14 | Engineered v6 | SPPS | 3 |
| H15 | Engineered v7 | DKTHTCAA | 16 |
| H16 | Engineered v8 | DKTHTCPPCPA | 17 |
| H17 | Engineered v9 | DKTHTCPPCPATCPPCPA | 18 |
| H18 | Engineered v10 | DKTHTCPPCPATCPPCPATC | 19 |
| | | PPCPA | |
| H19 | Engineered v11 | DKTHTCPPCPAGKPTLYNSL | 20 |
| | | VMSDTAGTCY | |
| H20 | Engineered v12 | DKTHTCPPCPAGKPTHVNVS | 21 |
| | | VVMAEVDGTCY | |
| H21 | Engineered v13 | DKTHTCCVECPPCPA | 22 |
| H22 | Engineered v14 | DKTHTCPRCPEPKSCDTPPP | 23 |
| | | CPRCPA | |
| H23 | Engineered v15 | DKTHTCPSCPA | 24 |

In one embodiment, the heavy chain Fc region possesses an intact hinge region at its N-terminus.

In one embodiment the heavy chain Fc region and hinge region are derived from IgG4 and the hinge region comprises the modified sequence CPPC (SEQ ID NO: 2). The core hinge region of human IgG4 contains the sequence CPSC (SEQ ID NO: 12) compared to IgG1 which contains the sequence CPPC (SEQ ID NO: 2). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

6.3.3. ABM Linkers

In certain aspects, the present disclosure provides TBMs comprising at least three ABMs, wherein two or more components of an ABM (e.g., a VH and a VL of an scFv), two or more ABMs, or an ABM and a non-ABM domain (e.g., a dimerization domain such as an Fc region) are connected to one another by a peptide linker. Such linkers are referred to herein an “ABM linkers”, as opposed to the ADC linkers used to attach drugs to TBMs as described, for example, in Section 6.9.2.

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids or from 12 to 20 amino acids. In particular embodiments, a peptide linker is 2 amino acids, 3 amino acids, 4 amino acid, 5 amino acids, 6 amino acids, 7 amino acids, 8 amino acids, 9 amino acids, 10 amino acids, 11 amino acids, 12 amino acids, 13 amino acids, 14 amino acid, 15 amino acids, 16 amino acids, 17 amino acids, 18 amino acids, 19 amino acids, 20 amino acids, 21 amino acids, 22 amino acids, 23 amino acids, 24 amino acid, 25 amino acids, 26 amino acids, 27 amino acids, 28 amino acids, 29 amino acids, 30 amino acids, 31 amino acids, 32 amino acids, 33 amino acids, 34 amino acid, 35 amino acids, 36 amino acids, 37 amino acids, 38 amino acids, 39 amino acids, 40 amino acids, 41 amino acids, 42 amino acids, 43 amino acids, 44 amino acid, 45 amino acids, 46 amino acids, 47 amino acids, 48 amino acids, 49 amino acids, or 50 amino acids in length.

Charged and/or flexible linkers are particularly preferred.

Examples of flexible ABM linkers that can be used in the TBMs of the disclosure include those disclosed by Chen et al., 2013, Adv Drug Deliv Rev. 65(10):1357-1369 and Klein et al., 2014, Protein Engineering, Design & Selection 27(10):325-330. A particularly useful flexible linker is (GGGGS)n (SEQ ID NO: 25) (also referred to as (G4S)n (SEQ ID NO: 25)). In some embodiments, n is any number between 1 and 10, i.e., 1, 2, 3, 4, 5, 6, 7, 8, 9, and 10, or any range bounded by any two of the foregoing numbers, e.g., 1 to 5, 2 to 5, 3 to 6, 2 to 4, 1 to 4, and so on and so forth.

Other examples of suitable TBM linkers for use in the TBMs of the present disclosure are shown in Table 4 below:

TABLE 4
ABM Linker Sequences

| Linker | | SEQ ID |
|---|---|---|
| Name | Linker Sequence | NO: |
| L1 | ADAAP | 26 |
| L2 | ADAAPTVSIFP | 27 |
| L3 | ADAAPTVSIFPP | 28 |
| L4 | AKTTAP | 29 |
| L5 | AKTTAPSVYPLAP | 30 |
| L6 | AKTTPKLEEGEFSEARV | 31 |
| L7 | AKTTPKLGG | 32 |
| L8 | AKTTPP | 33 |
| L9 | AKTTPPSVTPLAP | 34 |
| L10 | ASTKGP | 35 |
| L11 | ASTKGPSVFPLAP | 36 |
| L12 | ASTKGPSVFPLAPASTKGPSVFPLAP | 37 |
| L13 | EGKSSGSGSESKST | 38 |
| L14 | GEGESGEGESGEGES | 39 |
| L15 | GEGESGEGESGEGESGEGES | 40 |
| L16 | GEGGSGEGGSGEGGS | 41 |
| L17 | GENKVEYAPALMALS | 42 |
| L18 | GGEGSGGEGSGGEGS | 43 |
| L19 | GGGESGGEGSGEGGS | 44 |
| L20 | GGGESGGGESGGGES | 45 |
| L21 | (GGGGS)ₙ (also referred to as | 46 |
| | (G4S)ₙ), wherein n can be 1-10. | |
| L22 | GGGGSGGGGS | 47 |
| L23 | GGGGSGGGGSGGGGS | 1 |
| L24 | GGGGSGGGGSGGGGSGGGGS | 48 |
| L25 | GGGKSGGGKSGGGKS | 49 |
| L26 | GGGKSGGKGSGKGGS | 50 |
| L27 | GGKGSGGKGSGGKGS | 51 |
| L28 | GGSGG | 52 |
| L29 | GGSGGGGSG | 53 |
| L30 | GGSGGGGSGGGGS | 54 |
| L31 | GHEAAAVMQVQYPAS | 55 |
| L32 | GKGGSGKGGSGKGGS | 56 |
| L33 | GKGKSGKGKSGKGKS | 57 |
| L34 | GKGKSGKGKSGKGKSGKGKS | 58 |
| L35 | GKPGSGKPGSGKPGS | 59 |
| L36 | GKPGSGKPGSGKPGSGKPGS | 60 |
| L37 | GPAKELTPLKEAKVS | 61 |
| L38 | GSAGSAAGSGEF | 62 |
| L39 | IRPRAIGGSKPRVA | 63 |
| L40 | KESGSVSSEQLAQFRSLD | 64 |
| L41 | KTTPKLEEGEFSEAR | 65 |
| L42 | QPKAAP | 66 |
| L43 | QPKAAPSVTLFPP | 67 |
| L44 | RADAAAA(G4S)₄ | 68 |
| L45 | RADAAAAGGPGS | 69 |
| L46 | RADAAP | 70 |
| L47 | RADAAPTVS | 71 |
| L48 | SAKTTP | 72 |
| L49 | SAKTTPKLEEGEFSEARV | 73 |
| L50 | SAKTTPKLGG | 74 |
| L51 | STAGDTHLGGEDFD | 75 |
| L52 | TVAAP | 76 |
| L53 | TVAAPSVFIFPP | 77 |
| L54 | TVAAPSVFIFPPTVAAPSVFIFPP | 78 |

In various aspects, the disclosure provides a TBM which comprises one or more ABM linkers. Each of the ABM linkers can be range from 2 amino acids to 60 amino acids in length, preferably 4 to 30 amino acids, from 5 to 25 amino acids, from 10 to 25 amino acids or from 12 to 20 amino acids in length, optionally selected from Table 4 above. In particular embodiments, the TBM comprises two, three, four, five or six ABM linkers. The ABM linkers can be on one, two, three, four or even more polypeptide chains of the TBM.

6.4. Exemplary Trispecific Binding Molecules

Exemplary TBM configurations are shown in FIG. 1. FIG. 1A shows the components of the TBM configurations shown in FIGS. 1B-1Z. The scFv, Fab, non-immunoglobulin based ABM, and Fc each can have the characteristics described for these components in Sections 6.2 and 6.3. The components of the TBM configurations shown in FIG. 1 can be associated with each other by any of the means described in Sections 6.2 and 6.3 (e.g., by direct bonds, ABM linkers, disulfide bonds, Fc domains with modified with knob in hole interactions, etc.). The orientations and associations of the various components shown in FIG. 1 are merely exemplary; as will be appreciated by skilled artisans, other orientations and associations may be suitable (e.g., as described in Sections 6.2 and 6.3).

TBMs of the disclosure are not limited to the configurations shown in FIG. 1. Other configurations that may be used are known to those skilled in the art. See, e.g., WO 2014/145806; WO 2017/124002; Liu et al., 2017, Front Immunol. 8:38; Brinkmann & Kontermann, 2017, mAbs 9:2, 182-212; US 2016/0355600; Klein et al., 2016, MAbs 8(6):1010-20; and US 2017/0145116.

6.4.1. Exemplary Trivalent TBMs

The TBMs of the disclosure can be trivalent, i.e., they have three antigen-binding domains, each of which binds one of CD2, a component of a TCR complex and TAA.

Exemplary trivalent TBM configurations are shown in FIGS. 1B through 1O and FIGS. 1V-1Z.

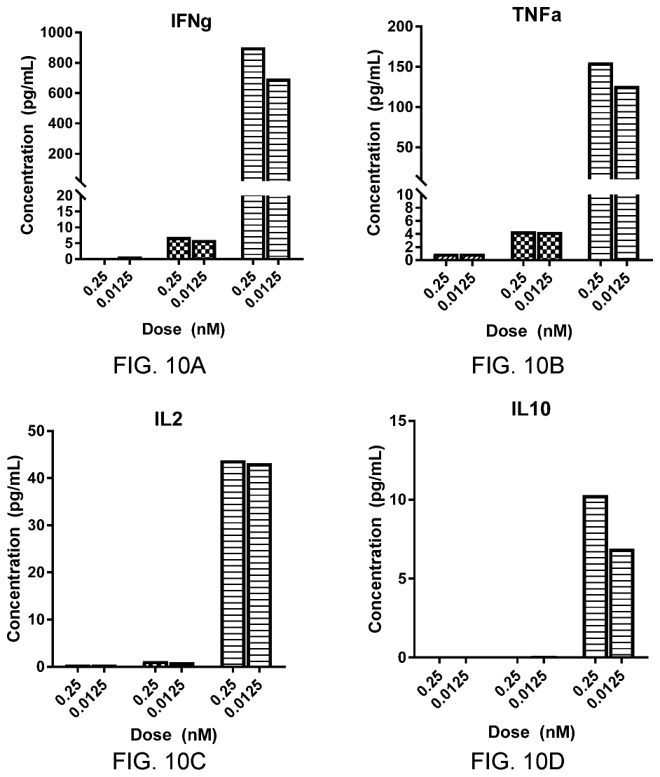

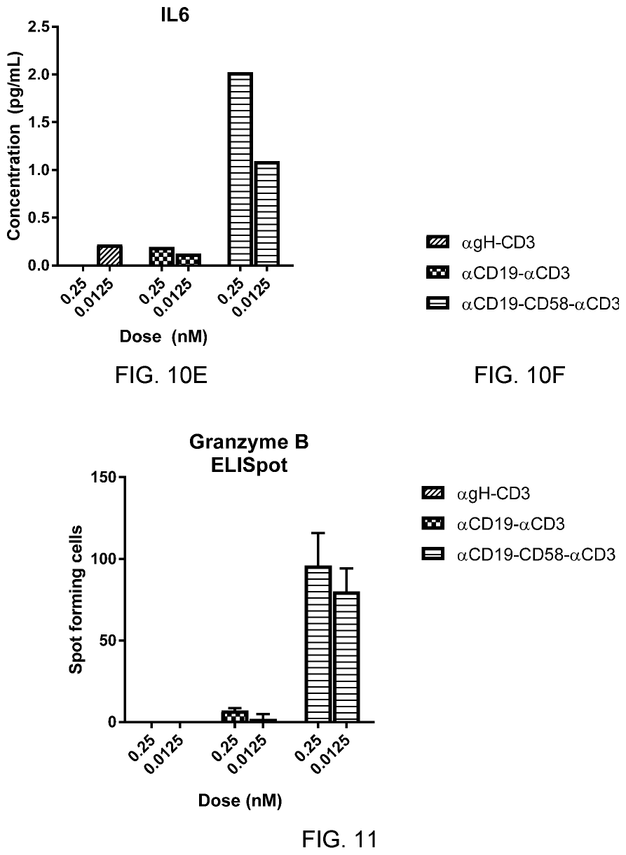

As depicted in FIGS. 1B-1K, 10 and 1V-1Z, a TBM can comprise two half antibodies, one comprising two ABMs and the other comprising one ABM, the two halves paired through an Fc domain.

In the embodiment of FIG. 1B, the first (or left) half antibody comprises an scFv and an Fc region, and the second (or right) half antibody comprises a Fab, an scFv and an Fc region. The first and second half antibodies are associated through the Fc regions forming an Fc domain.

In the embodiment of FIG. 1C, the first (or left) half antibody comprises two Fab and an Fc region, and the second (or right) half antibody comprises a Fab and an Fc region. The first and second half antibodies are associated through the Fc regions forming an Fc domain.

In the embodiment of FIG. 1D, the first (or left) half antibody comprises a Fab, an scFv and an Fc region, and the second (or right) half antibody comprises a Fab and an Fc region. The first and second half antibodies are associated through the Fc regions forming an Fc domain.

In the embodiment of FIG. 1E, the first (or left) half antibody comprises an scFv and an Fc region, and the second (or right) half antibody comprises two Fab and an Fc region. The first and second half antibodies are associated through the Fc regions forming an Fc domain.

In the embodiment of FIG. 1F, the first (or left) half antibody comprises an scFv, an Fc region, and a Fab, and the second (or right) half antibody comprises a Fab and an Fc region. The first and second half antibodies are associated through the Fc regions forming an Fc domain.

In the embodiment of FIG. 1G, the first (or left) half antibody comprises an scFv and an Fc region, and the second (or right) half antibody comprises a Fab an Fc region, and an scFV. The first and second half antibodies are associated through the Fc regions forming an Fc domain.

In the embodiment of FIG. 1H, the first (or left) half antibody comprises two Fab and an Fc region, and the second (or right) half antibody comprises a non-immunoglobulin based ABM and an Fc region. The first and second half antibodies are associated through the Fc regions forming an Fc domain.

In the embodiment of FIG. 1I, the first (or left) half antibody comprises a Fab, an scFv, and an Fc region, and the second (or right) half antibody comprises a non-immunoglobulin based ABM and an Fc region. The first and second half antibodies are associated through the Fc regions forming an Fc domain.

In the embodiment of FIG. 1J, the first (or left) half antibody comprises a Fab and an Fc region, and the second (or right) half antibody comprises an scFv, a non-immunoglobulin based ABM and an Fc region. The first and second half antibodies are associated through the Fc regions forming an Fc domain.

In the embodiment of FIG. 1K, the first (or left) half antibody comprises an scFv and an Fc region, and the second (or right) half antibody comprises an scFv, an Fc region, and a second scFv. The first and second half antibodies are associated through the Fc regions forming an Fc domain.

In the embodiment of FIG. 1N, the first (or left) half antibody comprises a Fab, an Fc region, and an scFv, and the second (or right) half antibody comprises a Fab, and an Fc region. The first and second half antibodies are associated through the Fc regions forming an Fc domain.

In the embodiment of FIG. 1O, the first (or left) half antibody comprises a Fab, an Fc region, and a scFab, and the second (or right) half antibody comprises a Fab and an Fc region. The first and second half antibodies are associated through the Fc regions forming an Fc domain.

In the embodiment of FIG. 1V, the first (or left) half antibody comprises a non-immunoglobulin based ABM, an Fab and an Fc region, and the second (or right) half antibody comprises an Fab and an Fc region. The first and second half antibodies are associated through the Fc regions forming an Fc domain.

In the embodiment of FIG. 1W, the first (or left) half antibody comprises a non-immunoglobulin based ABM, an scFv and an Fc region, and the second (or right) half antibody comprises an Fab and an Fc region. The first and second half antibodies are associated through the Fc regions forming an Fc domain.

In the embodiment of FIG. 1X, the first (or left) half antibody comprises an scFv, an Fc region and a non-immunoglobulin based ABM on the C-terminal end of the Fc region, and the second (or right) half antibody comprises an Fab and an Fc region. The first and second half antibodies are associated through the Fc regions forming an Fc domain.

In the embodiment of FIG. 1Y, the first (or left) half antibody comprises an scFv, an Fc region and an scFv on the C-terminal end of the Fc region, and the second (or right) half antibody comprises non-immunoglobulin based ABM and an Fc region. The first and second half antibodies are associated through the Fc regions forming an Fc domain.

In the embodiment of FIG. 1Z, the first (or left) half antibody comprises an scFv, an Fc region and an Fab on the C-terminal end of the Fc region, and the second (or right) half antibody comprises non-immunoglobulin based ABM and an Fc region. The first and second half antibodies are associated through the Fc regions forming an Fc domain.

Alternatively, as depicted in FIG. 1L, trivalent a TBM can comprise two half antibodies, each comprising one complete ABM and a portion of another ABM (one a VH, the other a VL). The two half antibodies are paired through an Fc domain, whereupon the VH and the VL associate to form a complete antigen-binding Fv domain.

The TBM can be a single chain, as shown in FIG. 1M. The TBM of FIG. 1M comprises three scFv domains connected through linkers.

In each of the configurations shown in FIGS. 1B-1O and 1V-Z, each of the domains designated X, Y, and Z represents a TCR ABM, a CD2 ABM, or a TAA ABM, although not necessarily in that order. In order words, X can be a TCR ABM, a CD2 ABM, or TAA ABM, Y can be a TCR ABM, a CD2 ABM, or a TAA ABM, and Z can be a CD2 ABM, a TCR ABM, or a TAA ABM, provided that the TBM comprises at least one TCR ABM, at least one CD2 ABM, and at least one TAA ABM.

Accordingly, in the present disclosure provides a trivalent TBM as shown in any one of FIGS. 1B through 1O and 1V through Z, wherein X is a CD2 ABM, Y is a TCR ABM and Z is a TAA ABM (this configuration of ABMs designated as “T1” for convenience).

The present disclosure also provides a trivalent TBM as shown in any one of FIGS. 1B through 1O and 1V through Z, wherein X is a CD2 ABM, Y is a TAA ABM, and Z is a TCR ABM (this configuration of ABMs designated as “T2” for convenience).

The present disclosure further provides a trivalent TBM as shown in any one of FIGS. 1B through 1O and 1V through Z, wherein X is a TCR ABM, Y is a CD2 ABM, and Z is a TAA ABM (this configuration of ABMs designated as “T3” for convenience).

The present disclosure yet further provides a trivalent TBM as shown in any one of FIGS. 1B through 1O and 1V through Z, wherein X is a TCR ABM, Y is a TAA ABM, and Z is a CD2 ABM (this configuration of ABMs designated as “T4” for convenience).

The present disclosure yet further provides a trivalent TBM as shown in any one of FIGS. 1B through 1O and 1V through Z, wherein X is a TAA ABM, Y is a CD2 ABM, and Z is a TCR ABM (this configuration of ABMs designated as “T5” for convenience).

The present disclosure yet further provides a trivalent TBM as shown in any one of FIGS. 1B through 1O and 1V through Z, wherein X is a TAA ABM, Y is a TCR ABM, and Z is a CD2 ABM (this configuration of ABMs designated as “T6” for convenience).

6.4.2. Exemplary Tetravalent TBMs

The TBMs of the disclosure can be tetravalent, i.e., they have four antigen-binding domains, one or two of which binds CD2, one or two of which binds a component of a TCR complex, and one or two of which binds TAA.

Exemplary tetravalent TBM configurations are shown in FIGS. 1P-1R.

As depicted in FIGS. 1P-1R, a tetravalent TBM can comprise two half antibodies, each comprising two complete ABMs, the two halves paired through an Fc domain.

In the embodiment of FIG. 1P, the first (or left) half antibody comprises a Fab, an Fc region, and a second Fab, and the second (or right) half antibody comprises a Fab, an Fc region, and a second Fab. The first and second half antibodies are associated through the Fc regions forming an Fc domain.

In the embodiment of FIG. 1Q, the first (or left) half antibody comprises a Fab, an Fc region, and an scFv, and the second (or right) half antibody comprises a Fab, an Fc region, and an scFv. The first and second half antibodies are associated through the Fc regions forming an Fc domain.

In the embodiment of FIG. 1R, the first (or left) half antibody comprises a Fab, an Fc region, and an scFv, and the second (or right) half antibody comprises an scFv, an Fc region, and a Fab. The first and second half antibodies are associated through the Fc regions forming an Fc domain.

In the configuration shown in FIGS. 1P-1R, each of X, Y, Z, and A represent a TCR ABM, a CD2 ABM, or a TAA ABM, although not necessarily in that order, and provided that the TBM comprises at least one TCR ABM, one CD2 ABM, and one TAA ABM. Thus, the tetravalent ABMs of the disclosure will include two ABMs against one of a component of a TCR complex, CD2 and TAA. Preferably, a tetravalent TBM has two TAA ABMs.

Accordingly, in the present disclosure provides a tetravalent TBM as shown in any one of FIGS. 1P-1R, wherein X is a CD2 ABM, Y is a TCR ABM and both Z and A are TAA ABMs (this configuration of ABMs designated as “Tv 1” for convenience).

The disclosure further provides a tetravalent TBM as shown in any one of FIGS. 1P-1R, wherein X is a CD2 ABM, both Y and A are TAA ABMs, and Z is a TCR ABM (this configuration of ABMs designated as “Tv 2” for convenience).

The disclosure yet further provides a tetravalent TBM as shown in any one of FIGS. 1P-1R, wherein X is a TCR ABM, Y is a CD2 ABM, and both Z and A are TAA ABMs (this configuration of ABMs designated as “Tv 3” for convenience).

The disclosure further provides a tetravalent TBM as shown in any one of FIGS. 1P-1R, wherein, X is a TCR ABM, both Y and A are TAA ABMs, and Z is a CD2 ABM (this configuration of ABMs designated as “Tv 4” for convenience).

The disclosure further provides a tetravalent TBM as shown in any one of FIGS. 1P-1R, wherein both X and A are TAA ABMs, Y is a CD2 ABM, and Z is a TCR ABM (this configuration of ABMs designated as “Tv 5” for convenience).

The disclosure further provides a tetravalent TBM as shown in any one of FIGS. 1P-1R, wherein both X and A are TAA ABMs, Y is a TCR ABM, and Z is a CD2 ABM (this configuration of ABMs designated as “Tv 6” for convenience).

The disclosure further provides a tetravalent TBM as shown in any one of FIGS. 1P-1R, wherein X is a CD2 ABM, both Y and Z are CD2 ABMs, and Z is a TCR ABM (this configuration of ABMs designated as “Tv 7” for convenience).

The disclosure further provides a tetravalent TBM as shown in any one of FIGS. 1P-1R, wherein both X and Z are TAA ABMs, Y is a CD2 ABM, and A is a TCR ABM (this configuration of ABMs designated as “Tv 8” for convenience).

The disclosure further provides a tetravalent TBM as shown in any one of FIGS. 1P-1R, wherein X is a TCR ABM, both Y and A are TAA ABMs, and Z is a CD2 ABM (this configuration of ABMs designated as “Tv 9” for convenience).

The disclosure further provides a tetravalent TBM as shown in any one of FIGS. 1P-1R, wherein both X and Y are TAA ABMs, Z is a CD2 ABM, and A is a TCR ABM (this configuration of ABMs designated as “Tv 10” for convenience).

The disclosure further provides a tetravalent TBM as shown in any one of FIGS. 1P-1R, wherein both X and Z are TAA ABMs, Y is a TCR ABM, and A is a CD2 ABM (this configuration of ABMs designated as “Tv 11” for convenience).

The disclosure further provides a tetravalent TBM as shown in any one of FIGS. 1P-1R, wherein both X and Y are TAA ABMs, Z is a TCR ABM, and A is a CD2 ABM (this configuration of ABMs designated as “Tv 12” for convenience).

6.4.3. Exemplary Pentavalent TBMs

The TBMs of the disclosure can be pentavalent, i.e., they have five antigen-binding domains, one, two, or three of which binds CD2, one, two, or three of which binds a component of a TCR complex, and one, two, or three of which binds TAA.

An exemplary pentavalent TBM configuration is shown in FIG. 15.

As depicted in FIG. 15, a pentavalent TBM can comprise two half antibodies, one of which comprises two complete ABMs and the other of which comprises one complete ABM, the two halves paired through an Fc domain.

In the embodiment of FIG. 15, the first (or left) half antibody comprises a Fab, an scFv, and an Fc region, and the second (or right) half antibody comprises a Fab, an Fc region, and an scFv. The first and second half antibodies are associated through the Fc regions forming an Fc domain.

In the configuration shown in FIG. 15, each of X, Y, Z, A, and B represent a TCR ABM, a CD2 ABM, or a TAA ABM, although not necessarily in that order, and provided that the TBM comprises at least one TCR ABM, one CD2 ABM, and one TAA ABM. Thus, the pentavalent TBMs of the disclosure can include two ABMs against two of a component of a TCR complex, CD2 and TAA, or three ABMs against one of a component of a TCR complex, CD2 and TAA. Preferably, a pentavalent TBM has two or three TAA ABMs. In a specific embodiment, a pentavalent TBM has three TAA ABMs.

Accordingly, the present disclosure provides a pentavalent TBM as shown in FIG. 15, wherein X, Y, Z, A, and B are ABMs directed to CD2, a component of a TCR complex, and TAA as shown in Table 5.

TABLE 5
ABM Permutations in Pentavalent TBMs

| Pentavalent | | | | | |
|---|---|---|---|---|---|
| Configuration | X | Y | Z | A | B |
| Pv 1 | CD2 | CD2 | TCR | TAA | TAA |
| Pv 2 | CD2 | CD2 | TAA | TCR | TAA |
| Pv 3 | CD2 | CD2 | TAA | TAA | TCR |
| Pv 4 | CD2 | TCR | CD2 | TAA | TAA |
| Pv 5 | CD2 | TAA | CD2 | TCR | TAA |
| Pv 6 | CD2 | TAA | CD2 | TAA | TCR |
| Pv 7 | CD2 | TCR | TAA | CD2 | TAA |
| Pv 8 | CD2 | TAA | TCR | CD2 | TAA |
| Pv 9 | CD2 | TAA | TAA | CD2 | TCR |
| Pv 10 | CD2 | TCR | TAA | TAA | CD2 |
| Pv 11 | CD2 | TAA | TCR | TAA | CD2 |
| Pv 12 | CD2 | TAA | TAA | TCR | CD2 |
| Pv 13 | TCR | CD2 | CD2 | TAA | TAA |
| Pv 14 | TAA | CD2 | CD2 | TCR | TAA |
| Pv 15 | TAA | CD2 | CD2 | TAA | TCR |
| Pv 16 | TCR | CD2 | TAA | CD2 | TAA |
| Pv 17 | TAA | CD2 | TCR | CD2 | TAA |
| Pv 18 | TAA | CD2 | TAA | CD2 | TCR |
| Pv 19 | TCR | CD2 | TAA | TAA | CD2 |
| Pv 20 | TAA | CD2 | TCR | TAA | CD2 |
| Pv 21 | TAA | CD2 | TAA | TCR | CD2 |
| Pv 22 | TCR | TAA | CD2 | CD2 | TAA |
| Pv 23 | TAA | TCR | CD2 | CD2 | TAA |
| Pv 24 | TAA | TAA | CD2 | CD2 | TCR |
| Pv 25 | TCR | TAA | CD2 | TAA | CD2 |
| Pv 26 | TAA | TCR | CD2 | TAA | CD2 |
| Pv 27 | TAA | TAA | CD2 | TCR | CD2 |
| Pv P28 | TCR | TAA | TAA | CD2 | CD2 |
| Pv 29 | TAA | TCR | TAA | CD2 | CD2 |
| Pv 30 | TAA | TAA | TCR | CD2 | CD2 |
| Pv 31 | CD2 | TCR | TCR | TAA | TAA |
| Pv 32 | CD2 | TCR | TAA | TCR | TAA |
| Pv 33 | CD2 | TCR | TAA | TAA | TCR |
| Pv 34 | CD2 | TAA | TCR | TCR | TAA |
| Pv 35 | CD2 | TAA | TCR | TAA | TCR |
| Pv 36 | CD2 | TAA | TAA | TCR | TCR |
| Pv 37 | TCR | CD2 | TCR | TAA | TAA |
| Pv 38 | TCR | CD2 | TAA | TCR | TAA |
| Pv 39 | TCR | CD2 | TAA | TAA | TCR |
| Pv 40 | TAA | CD2 | TCR | TCR | TAA |
| Pv 41 | TAA | CD2 | TCR | TAA | TCR |
| Pv 42 | TAA | CD2 | TAA | TCR | TCR |
| Pv 43 | TCR | TCR | CD2 | TAA | TAA |
| Pv 44 | TCR | TAA | CD2 | TCR | TAA |
| Pv 45 | TCR | TAA | CD2 | TAA | TCR |
| Pv 46 | TAA | TCR | CD2 | TCR | TAA |
| Pv 47 | TAA | TCR | CD2 | TAA | TCR |
| Pv 48 | TAA | TAA | CD2 | TCR | TCR |
| Pv 49 | TCR | TCR | TAA | CD2 | TAA |
| Pv 50 | TCR | TAA | TCR | CD2 | TAA |
| Pv 51 | TCR | TAA | TAA | CD2 | TCR |
| Pv 52 | TAA | TCR | TCR | CD2 | TAA |
| Pv 53 | TAA | TCR | TAA | CD2 | TCR |
| Pv 54 | TAA | TAA | TCR | CD2 | TCR |
| Pv 55 | TCR | TCR | TAA | TAA | CD2 |
| Pv 56 | TCR | TAA | TCR | TAA | CD2 |
| Pv 57 | TCR | TAA | TAA | TCR | CD2 |
| Pv 58 | TAA | TCR | TCR | TAA | CD2 |
| Pv 59 | TAA | TCR | TAA | TCR | CD2 |
| Pv 60 | TAA | TAA | TCR | TCR | CD2 |
| Pv 61 | CD2 | TCR | TAA | TAA | TAA |
| Pv 62 | CD2 | TAA | TCR | TAA | TAA |
| Pv 63 | CD2 | TAA | TAA | TCR | TAA |
| Pv 64 | CD2 | TAA | TAA | TAA | TCR |
| Pv 65 | TCR | CD2 | TAA | TAA | TAA |
| Pv 66 | TAA | CD2 | TCR | TAA | TAA |
| Pv 67 | TAA | CD2 | TAA | TCR | TAA |
| Pv 68 | TAA | CD2 | TAA | TAA | TCR |
| Pv 69 | TCR | TAA | CD2 | TAA | TAA |
| Pv 70 | TAA | TCR | CD2 | TAA | TAA |
| Pv 71 | TAA | TAA | CD2 | TCR | TAA |
| Pv 72 | TAA | TAA | CD2 | TAA | TCR |
| Pv 73 | TCR | TAA | TAA | CD2 | TAA |
| Pv 74 | TAA | TCR | TAA | CD2 | TAA |
| Pv 75 | TAA | TAA | TCR | CD2 | TAA |
| Pv 76 | TAA | TAA | TAA | CD2 | TCR |
| Pv 77 | TCR | TAA | TAA | TAA | CD2 |
| Pv 78 | TAA | TCR | TAA | TAA | CD2 |
| Pv 79 | TAA | TAA | TCR | TAA | CD2 |
| Pv 80 | TAA | TAA | TAA | TCR | CD2 |

6.4.4. Exemplary Hexavalent TBMs

The TBMs of the disclosure can be hexavalent, i.e., they have six antigen-binding domains, one, two, three, or four of which binds CD2, one, two, three, or four of which binds a component of a TCR complex, and one, two, three, or four of which binds TAA.

Exemplary hexavalent TBM configurations are shown in FIGS. 1T-1U.

As depicted in FIGS. 1T-1U, a pentavalent TBM can comprise two half antibodies, one of which comprises two complete ABMs and the other of which comprises one complete ABM, the two halves paired through an Fc domain.

In the embodiment of FIG. 1T, the first (or left) half antibody comprises a Fab, a second Fab, an Fc region, and an scFv, and the second (or right) half antibody comprises a Fab, a second Fab, an Fc region, and an scFv. The first and second half antibodies are associated through the Fc regions forming an Fc domain.

In the embodiment of FIG. 1U, the first (or left) half antibody comprises a first Fv, a second Fv, a third Fv, and an Fc region, and the second (or right) half antibody comprises a first Fv, a second Fv, a third Fv, and an Fc region. The first and second half antibodies are associated through the Fc regions forming an Fc domain.

In the configuration shown in FIGS. 1T-1U, each of X, Y, Z, A, B, and C represent a TCR ABM, a CD2 ABM, or a TAA ABM, although not necessarily in that order, and provided that the TBM comprises at least one TCR ABM, one CD2 ABM, and one TAA ABM. Thus, the hexavalent TBMs of the disclosure can include (i) two ABMs against each of a component of a TCR complex, CD2 and TAA, (ii) three ABMs against one of a component of a TCR complex, CD2 and TAA, (iii) or four ABMs against one of a component of a TCR complex, CD2, and TAA. For example, a hexavalent ABM can include three ABMs against TAA, two ABMs against CD2 and one ABM against a component of a TCR complex. As another example, a hexavalent ABM can include three ABMs against TAA, two ABMs against a component of a TCR complex and one ABM against CD2. Preferably, a hexavalent TBM has two, three, our four TAA ABMs. In a specific embodiment, a hexavalent TBM has three TAA ABMs. In other embodiments, a hexavalent TBM has four TAA ABMs.

Accordingly, in the present disclosure provides hexavalent TBMs as shown in any one of FIGS. 1T-1U, wherein X, Y, Z, A, B, and C are ABMs directed to CD2, a component of a TCR complex, and TAA as shown in Table 6.

TABLE 6
ABM Permutations in Hexavalent TBMs

| Hexa- | | | | | | |
|---|---|---|---|---|---|---|
| valent | | | | | | |
| Config- | | | | | | |
| uration | X | Y | Z | A | B | C |
| Hv 1 | CD2 | CD2 | TCR | TCR | TAA | TAA |
| Hv 2 | CD2 | CD2 | TCR | TAA | TCR | TAA |
| Hv 3 | CD2 | CD2 | TCR | TAA | TAA | TCR |
| Hv 4 | CD2 | CD2 | TAA | TCR | TCR | TAA |
| Hv 5 | CD2 | CD2 | TAA | TCR | TAA | TCR |
| Hv 6 | CD2 | CD2 | TAA | TAA | TCR | TCR |
| Hv 7 | CD2 | TCR | CD2 | TCR | TAA | TAA |
| Hv 8 | CD2 | TCR | CD2 | TAA | TCR | TAA |
| Hv 9 | CD2 | TCR | CD2 | TAA | TAA | TCR |
| Hv 10 | CD2 | TAA | CD2 | TCR | TCR | TAA |
| Hv 11 | CD2 | TAA | CD2 | TCR | TAA | TCR |
| Hv 12 | CD2 | TAA | CD2 | TAA | TCR | TCR |
| Hv 13 | CD2 | TCR | TCR | CD2 | TAA | TAA |
| Hv 14 | CD2 | TCR | TAA | CD2 | TCR | TAA |
| Hv 15 | CD2 | TCR | TAA | CD2 | TAA | TCR |
| Hv 16 | CD2 | TAA | TCR | CD2 | TCR | TAA |
| Hv 17 | CD2 | TAA | TCR | CD2 | TAA | TCR |
| Hv 18 | CD2 | TAA | TAA | CD2 | TCR | TCR |
| Hv 19 | CD2 | TCR | TCR | TAA | CD2 | TAA |
| Hv 20 | CD2 | TCR | TAA | TCR | CD2 | TAA |
| Hv 21 | CD2 | TCR | TAA | TAA | CD2 | TCR |
| Hv 22 | CD2 | TAA | TCR | TCR | CD2 | TAA |
| Hv 23 | CD2 | TAA | TCR | TAA | CD2 | TCR |
| Hv 24 | CD2 | TAA | TAA | TCR | CD2 | TCR |
| Hv 25 | CD2 | TCR | TCR | TAA | TAA | CD2 |
| Hv 26 | CD2 | TCR | TAA | TCR | TAA | CD2 |
| Hv 27 | CD2 | TCR | TAA | TAA | TCR | CD2 |
| Hv 28 | CD2 | TAA | TCR | TCR | TAA | CD2 |
| Hv 29 | CD2 | TAA | TCR | TAA | TCR | CD2 |
| Hv 30 | CD2 | TAA | TAA | TCR | TCR | CD2 |
| Hv 31 | TCR | CD2 | CD2 | TCR | TAA | TAA |
| Hv 32 | TCR | CD2 | CD2 | TAA | TCR | TAA |
| Hv 33 | TCR | CD2 | CD2 | TAA | TAA | TCR |
| Hv 34 | TAA | CD2 | CD2 | TCR | TCR | TAA |
| Hv 35 | TAA | CD2 | CD2 | TCR | TAA | TCR |
| Hv 36 | TAA | CD2 | CD2 | TAA | TCR | TCR |
| Hv 37 | TCR | CD2 | TCR | CD2 | TAA | TAA |
| Hv 38 | TCR | CD2 | TAA | CD2 | TCR | TAA |
| Hv 39 | TCR | CD2 | TAA | CD2 | TAA | TCR |
| Hv 40 | TAA | CD2 | TCR | CD2 | TCR | TAA |
| Hv 41 | TAA | CD2 | TCR | CD2 | TAA | TCR |
| Hv 42 | TAA | CD2 | TAA | CD2 | TCR | TCR |
| Hv 43 | TCR | CD2 | TCR | TAA | CD2 | TAA |
| Hv 44 | TCR | CD2 | TAA | TCR | CD2 | TAA |
| Hv 45 | TCR | CD2 | TAA | TAA | CD2 | TCR |
| Hv 46 | TAA | CD2 | TCR | TCR | CD2 | TAA |
| Hv 47 | TAA | CD2 | TCR | TAA | CD2 | TCR |
| Hv 48 | TAA | CD2 | TAA | TCR | CD2 | TCR |
| Hv 49 | TCR | CD2 | TCR | TAA | TAA | CD2 |
| Hv 50 | TCR | CD2 | TAA | TCR | TAA | CD2 |
| Hv 51 | TCR | CD2 | TAA | TAA | TCR | CD2 |
| Hv 52 | TAA | CD2 | TCR | TCR | TAA | CD2 |
| Hv 53 | TAA | CD2 | TCR | TAA | TCR | CD2 |
| Hv 54 | TAA | CD2 | TAA | TCR | TCR | CD2 |
| Hv 55 | TCR | TCR | CD2 | CD2 | TAA | TAA |
| Hv 56 | TCR | TAA | CD2 | CD2 | TCR | TAA |
| Hv 57 | TCR | TAA | CD2 | CD2 | TAA | TCR |
| Hv 58 | TAA | TCR | CD2 | CD2 | TCR | TAA |
| Hv 59 | TAA | TCR | CD2 | CD2 | TAA | TCR |
| Hv 60 | TAA | TAA | CD2 | CD2 | TCR | TCR |
| Hv 61 | TCR | TCR | CD2 | TAA | CD2 | TAA |
| Hv 62 | TCR | TAA | CD2 | TCR | CD2 | TAA |
| Hv 63 | TCR | TAA | CD2 | TAA | CD2 | TCR |
| Hv 64 | TAA | TCR | CD2 | TCR | CD2 | TAA |
| Hv 65 | TAA | TCR | CD2 | TAA | CD2 | TCR |
| Hv 66 | TAA | TAA | CD2 | TCR | CD2 | TCR |
| Hv 67 | TCR | TCR | CD2 | TAA | TAA | CD2 |
| Hv 68 | TCR | TAA | CD2 | TCR | TAA | CD2 |
| Hv 69 | TCR | TAA | CD2 | TAA | TCR | CD2 |
| Hv 70 | TAA | TCR | CD2 | TCR | TAA | CD2 |
| Hv 71 | TAA | TCR | CD2 | TAA | TCR | CD2 |
| Hv 72 | TAA | TAA | CD2 | TCR | TCR | CD2 |
| Hv 73 | TCR | TCR | TAA | CD2 | CD2 | TAA |
| Hv 74 | TCR | TAA | TCR | CD2 | CD2 | TAA |
| Hv 75 | TCR | TAA | TAA | CD2 | CD2 | TCR |
| Hv 76 | TAA | TCR | TCR | CD2 | CD2 | TAA |
| Hv 77 | TAA | TCR | TAA | CD2 | CD2 | TCR |
| Hv 78 | TAA | TAA | TCR | CD2 | CD2 | TCR |
| Hv 79 | TCR | TCR | TAA | CD2 | TAA | CD2 |
| Hv 80 | TCR | TAA | TCR | CD2 | TAA | CD2 |
| Hv 81 | TCR | TAA | TAA | CD2 | TCR | CD2 |
| Hv 82 | TAA | TCR | TCR | CD2 | TAA | CD2 |
| Hv 83 | TAA | TCR | TAA | CD2 | TCR | CD2 |
| Hv 84 | TAA | TAA | TCR | CD2 | TCR | CD2 |
| Hv 85 | TCR | TCR | TAA | TAA | CD2 | CD2 |
| Hv 86 | TCR | TAA | TCR | TAA | CD2 | CD2 |
| Hv 87 | TCR | TAA | TAA | TCR | CD2 | CD2 |
| Hv 88 | TAA | TCR | TCR | TAA | CD2 | CD2 |
| Hv 89 | TAA | TCR | TAA | TCR | CD2 | CD2 |
| Hv 90 | TAA | TAA | TCR | TCR | CD2 | CD2 |
| Hv 91 | CD2 | TCR | TCR | TAA | TAA | TAA |
| Hv 92 | CD2 | TCR | TAA | TCR | TAA | TAA |
| Hv 93 | CD2 | TCR | TAA | TAA | TCR | TAA |
| Hv 94 | CD2 | TCR | TAA | TAA | TAA | TCR |
| Hv 95 | CD2 | TAA | TCR | TCR | TAA | TAA |
| Hv 96 | CD2 | TAA | TCR | TAA | TCR | TAA |
| Hv 97 | CD2 | TAA | TCR | TAA | TAA | TCR |
| Hv 98 | CD2 | TAA | TAA | TCR | TCR | TAA |
| Hv 99 | CD2 | TAA | TAA | TCR | TAA | TCR |
| Hv 100 | CD2 | TAA | TAA | TAA | TCR | TCR |
| Hv 101 | TCR | CD2 | TCR | TAA | TAA | TAA |
| Hv 102 | TCR | CD2 | TAA | TCR | TAA | TAA |
| Hv 103 | TCR | CD2 | TAA | TAA | TCR | TAA |
| Hv 104 | TCR | CD2 | TAA | TAA | TAA | TCR |
| Hv 105 | TAA | CD2 | TCR | TCR | TAA | TAA |
| Hv 106 | TAA | CD2 | TCR | TAA | TCR | TAA |
| Hv 107 | TAA | CD2 | TCR | TAA | TAA | TCR |
| Hv 108 | TAA | CD2 | TAA | TCR | TCR | TAA |
| Hv 109 | TAA | CD2 | TAA | TCR | TAA | TCR |
| Hv 110 | TAA | CD2 | TAA | TAA | TCR | TCR |
| Hv 111 | TCR | TCR | CD2 | TAA | TAA | TAA |
| Hv 112 | TCR | TAA | CD2 | TCR | TAA | TAA |
| Hv 113 | TCR | TAA | CD2 | TAA | TCR | TAA |
| Hv 114 | TCR | TAA | CD2 | TAA | TAA | TCR |
| Hv 115 | TAA | TCR | CD2 | TCR | TAA | TAA |
| Hv 116 | TAA | TCR | CD2 | TAA | TCR | TAA |
| Hv 117 | TAA | TCR | CD2 | TAA | TAA | TCR |
| Hv 118 | TAA | TAA | CD2 | TCR | TCR | TAA |
| Hv 119 | TAA | TAA | CD2 | TCR | TAA | TCR |
| Hv 120 | TAA | TAA | CD2 | TAA | TCR | TCR |
| Hv 121 | TCR | TCR | TAA | CD2 | TAA | TAA |
| Hv 122 | TCR | TAA | TCR | CD2 | TAA | TAA |
| Hv 123 | TCR | TAA | TAA | CD2 | TCR | TAA |
| Hv 124 | TCR | TAA | TAA | CD2 | TAA | TCR |
| Hv 125 | TAA | TCR | TCR | CD2 | TAA | TAA |
| Hv 126 | TAA | TCR | TAA | CD2 | TCR | TAA |
| Hv 127 | TAA | TCR | TAA | CD2 | TAA | TCR |
| Hv 128 | TAA | TAA | TCR | CD2 | TCR | TAA |
| Hv 129 | TAA | TAA | TCR | CD2 | TAA | TCR |
| Hv 130 | TAA | TAA | TAA | CD2 | TCR | TCR |
| Hv 131 | TCR | TCR | TAA | TAA | CD2 | TAA |
| Hv 132 | TCR | TAA | TCR | TAA | CD2 | TAA |
| Hv 133 | TCR | TAA | TAA | TCR | CD2 | TAA |
| Hv 134 | TCR | TAA | TAA | TAA | CD2 | TCR |
| Hv 135 | TAA | TCR | TCR | TAA | CD2 | TAA |
| Hv 136 | TAA | TCR | TAA | TCR | CD2 | TAA |
| Hv 137 | TAA | TCR | TAA | TAA | CD2 | TCR |
| Hv 138 | TAA | TAA | TCR | TCR | CD2 | TAA |
| Hv 139 | TAA | TAA | TCR | TAA | CD2 | TCR |
| Hv 140 | TAA | TAA | TAA | TCR | CD2 | TCR |
| Hv 141 | TCR | TCR | TAA | TAA | TAA | CD2 |
| Hv 142 | TCR | TAA | TCR | TAA | TAA | CD2 |
| Hv 143 | TCR | TAA | TAA | TCR | TAA | CD2 |
| Hv 144 | TCR | TAA | TAA | TAA | TCR | CD2 |
| Hv 145 | TAA | TCR | TCR | TAA | TAA | CD2 |
| Hv 146 | TAA | TCR | TAA | TCR | TAA | CD2 |
| Hv 147 | TAA | TCR | TAA | TAA | TCR | CD2 |
| Hv 148 | TAA | TAA | TCR | TCR | TAA | CD2 |
| Hv 149 | TAA | TAA | TCR | TAA | TCR | CD2 |
| Hv 150 | TAA | TAA | TAA | TCR | TCR | CD2 |
| Hv 151 | CD2 | CD2 | TCR | TAA | TAA | TAA |
| Hv 152 | CD2 | CD2 | TAA | TCR | TAA | TAA |
| Hv 153 | CD2 | CD2 | TAA | TAA | TCR | TAA |
| Hv 154 | CD2 | CD2 | TAA | TAA | TAA | TCR |
| Hv 155 | CD2 | TCR | CD2 | TAA | TAA | TAA |
| Hv 156 | CD2 | TAA | CD2 | TCR | TAA | TAA |
| Hv 157 | CD2 | TAA | CD2 | TAA | TCR | TAA |
| Hv 158 | CD2 | TAA | CD2 | TAA | TAA | TCR |
| Hv 159 | CD2 | TCR | TAA | CD2 | TAA | TAA |
| Hv 160 | CD2 | TAA | TCR | CD2 | TAA | TAA |
| Hv 161 | CD2 | TAA | TAA | CD2 | TCR | TAA |
| Hv 162 | CD2 | TAA | TAA | CD2 | TAA | TCR |
| Hv 163 | CD2 | TCR | TAA | TAA | CD2 | TAA |
| Hv 164 | CD2 | TAA | TCR | TAA | CD2 | TAA |
| Hv 165 | CD2 | TAA | TAA | TCR | CD2 | TAA |
| Hv 166 | CD2 | TAA | TAA | TAA | CD2 | TCR |
| Hv 167 | CD2 | TCR | TAA | TAA | TAA | CD2 |
| Hv 168 | CD2 | TAA | TCR | TAA | TAA | CD2 |
| Hv 169 | CD2 | TAA | TAA | TCR | TAA | CD2 |
| Hv 170 | CD2 | TAA | TAA | TAA | TCR | CD2 |
| Hv 171 | TCR | CD2 | CD2 | TAA | TAA | TAA |
| Hv 172 | TAA | CD2 | CD2 | TCR | TAA | TAA |
| Hv 173 | TAA | CD2 | CD2 | TAA | TCR | TAA |
| Hv 174 | TAA | CD2 | CD2 | TAA | TAA | TCR |
| Hv 175 | TCR | CD2 | TAA | CD2 | TAA | TAA |
| Hv 176 | TAA | CD2 | TCR | CD2 | TAA | TAA |
| Hv 177 | TAA | CD2 | TAA | CD2 | TCR | TAA |
| Hv 178 | TAA | CD2 | TAA | CD2 | TAA | TCR |
| Hv 179 | TCR | CD2 | TAA | TAA | CD2 | TAA |
| Hv 180 | TAA | CD2 | TCR | TAA | CD2 | TAA |
| Hv 181 | TAA | CD2 | TAA | TCR | CD2 | TAA |
| Hv 182 | TAA | CD2 | TAA | TAA | CD2 | TCR |
| Hv 183 | TCR | CD2 | TAA | TAA | TAA | CD2 |
| Hv 184 | TAA | CD2 | TCR | TAA | TAA | CD2 |
| Hv 185 | TAA | CD2 | TAA | TCR | TAA | CD2 |
| Hv 186 | TAA | CD2 | TAA | TAA | TCR | CD2 |
| Hv 187 | TCR | TAA | CD2 | CD2 | TAA | TAA |
| Hv 188 | TAA | TCR | CD2 | CD2 | TAA | TAA |
| Hv 189 | TAA | TAA | CD2 | CD2 | TCR | TAA |
| Hv 190 | TAA | TAA | CD2 | CD2 | TAA | TCR |
| Hv 191 | TCR | TAA | CD2 | TAA | CD2 | TAA |
| Hv 192 | TAA | TCR | CD2 | TAA | CD2 | TAA |
| Hv 193 | TAA | TAA | CD2 | TCR | CD2 | TAA |
| Hv 194 | TAA | TAA | CD2 | TAA | CD2 | TCR |
| Hv 195 | TCR | TAA | CD2 | TAA | TAA | CD2 |
| Hv 196 | TAA | TCR | CD2 | TAA | TAA | CD2 |
| Hv 197 | TAA | TAA | CD2 | TCR | TAA | CD2 |
| Hv 198 | TAA | TAA | CD2 | TAA | TCR | CD2 |
| Hv 199 | TCR | TAA | TAA | CD2 | CD2 | TAA |
| Hv 200 | TAA | TCR | TAA | CD2 | CD2 | TAA |
| Hv 201 | TAA | TAA | TCR | CD2 | CD2 | TAA |
| Hv 202 | TAA | TAA | TAA | CD2 | CD2 | TCR |
| Hv 203 | TCR | TAA | TAA | CD2 | TAA | CD2 |
| Hv 204 | TAA | TCR | TAA | CD2 | TAA | CD2 |
| Hv 205 | TAA | TAA | TCR | CD2 | TAA | CD2 |
| Hv 206 | TAA | TAA | TAA | CD2 | TCR | CD2 |
| Hv 207 | TCR | TAA | TAA | TAA | CD2 | CD2 |
| Hv 208 | TAA | TCR | TAA | TAA | CD2 | CD2 |
| Hv 209 | TAA | TAA | TCR | TAA | CD2 | CD2 |
| Hv 210 | TAA | TAA | TAA | TCR | CD2 | CD2 |
| Hv 211 | CD2 | TCR | TAA | TAA | TAA | TAA |
| Hv 212 | CD2 | TAA | TCR | TAA | TAA | TAA |
| Hv 213 | CD2 | TAA | TAA | TCR | TAA | TAA |
| Hv 214 | CD2 | TAA | TAA | TAA | TCR | TAA |
| Hv 215 | CD2 | TAA | TAA | TAA | TAA | TCR |
| Hv 216 | TCR | CD2 | TAA | TAA | TAA | TAA |
| Hv 217 | TAA | CD2 | TCR | TAA | TAA | TAA |
| Hv 218 | TAA | CD2 | TAA | TCR | TAA | TAA |
| Hv 219 | TAA | CD2 | TAA | TAA | TCR | TAA |
| Hv 220 | TAA | CD2 | TAA | TAA | TAA | TCR |
| Hv 221 | TCR | TAA | CD2 | TAA | TAA | TAA |
| Hv 222 | TAA | TCR | CD2 | TAA | TAA | TAA |
| Hv 223 | TAA | TAA | CD2 | TCR | TAA | TAA |
| Hv 224 | TAA | TAA | CD2 | TAA | TCR | TAA |
| Hv 225 | TAA | TAA | CD2 | TAA | TAA | TCR |
| Hv 226 | TCR | TAA | TAA | CD2 | TAA | TAA |
| Hv 227 | TAA | TCR | TAA | CD2 | TAA | TAA |
| Hv 228 | TAA | TAA | TCR | CD2 | TAA | TAA |
| Hv 229 | TAA | TAA | TAA | CD2 | TCR | TAA |
| Hv 230 | TAA | TAA | TAA | CD2 | TAA | TCR |
| Hv 231 | TCR | TAA | TAA | TAA | CD2 | TAA |
| Hv 232 | TAA | TCR | TAA | TAA | CD2 | TAA |
| Hv 233 | TAA | TAA | TCR | TAA | CD2 | TAA |
| Hv 234 | TAA | TAA | TAA | TCR | CD2 | TAA |
| Hv 235 | TAA | TAA | TAA | TAA | CD2 | TCR |
| Hv 236 | TCR | TAA | TAA | TAA | TAA | CD2 |
| Hv 237 | TAA | TCR | TAA | TAA | TAA | CD2 |
| Hv 238 | TAA | TAA | TCR | TAA | TAA | CD2 |
| Hv 239 | TAA | TAA | TAA | TCR | TAA | CD2 |
| Hv 240 | TAA | TAA | TAA | TAA | TCR | CD2 |

### 6.5. TCR ABMs

The TBMs of the disclosure contain an ABM that specifically binds to a component of a TCR complex. The TCR is a disulfide-linked membrane-anchored heterodimeric protein normally consisting of the highly variable alpha (α) and beta (β) chains expressed as part of a complex with the invariant CD3 chain molecules. T cells expressing this receptor are referred to as α:β (or ββ) T cells, though a minority of T cells express an alternate receptor, formed by variable gamma (γ) and delta (δ) chains, referred as γδ T cells.

In a preferred embodiment, TBMs of the disclosure contain an ABM that specifically binds to CD3.

6.5.1. CD3 ABMs

The TBMs of the disclosure can contain an ABM that specifically binds to CD3. The term “CD3” refers to the cluster of differentiation 3 co-receptor (or co-receptor complex, or polypeptide chain of the co-receptor complex) of the T cell receptor. The amino acid sequence of the polypeptide chains of human CD3 are provided in NCBI Accession P04234, P07766 and P09693. CD3 proteins may also include variants. CD3 proteins may also include fragments. CD3 proteins also include post-translational modifications of the CD3 amino acid sequences. Post-translational modifications include, but are not limited to, N- and O-linked glycosylation.

In some embodiments, a TBM of the disclosure can comprise an ABM which is an anti-CD3 antibody (e.g., as described in US 2016/0355600, WO 2014/110601, and WO 2014/145806, the contents of which are hereby incorporated by reference) or an antigen-binding domain thereof. Exemplary anti-CD3 VH, VL, and scFV sequences that can be used in TBM of the disclosure are provided in Table 7A.

TABLE 7A
CD3 Binders-Variable domain sequences

| | | | SEQ |
|---|---|---|---|
| Binding | | | ID |
| Domain | Chain | Sequence | NO: |
| CD3-1 | VH | QVQLQQSGAELARPGASVKMSCKASGYTFTRYTMHWVKQRPGQG | 79 |
| | | LEWIGYINPSRGYTNYNQKFKDKATLTTDKSSSTAYMQLSSLTSEDS | |
| | | AVYYCARYYDDHYCLDYWGQGTTLTVSS | |
| | VL | QIVLTQSPAIMSASPGEKVTMTCSASSSVSYMNWYQQKSGTSPKR | 80 |
| | | WIYDTSKLASGVPAHFRGSGSGTSYSLTISGMEAEDAATYYCQQWS | |
| | | SNPFTFGSGTKLEIN | |
| CD3-2 | VH | EVQLVESGGGLVQPKGSLKLSCAASGFTFNTYAMNWVRQAPGKGL | 81 |
| | | EWVARIRSKYNNYATYYADSVKDRFTISRDDSQSILYLQMNNLKTED | |
| | | TAMYYCVRHGNFGNSYVSWFAYWGQGTLVTVSA | |
| | VL | QAVVTQESALTTSPGETVTLTCRSSTGAVTTSNYANWVQEKPDHLF | 82 |
| | | TGLIGGTNKRAPGVPARFSGSLIGDKAALTITGAQTEDEAIYFCALWY | |
| | | SNLWVFGGGTKLTVL | |
| CD3-3 | VH | QVQLQQSGAELARPGASVKMSCKASGYTFTSYTMHWVKQRPGQG | 83 |
| | | LEWIGYINPSSGYTKYNQKFKDKATLTADKSSSTAYMQLSSLTSEDS | |
| | | AVYYCARWQDYDVYFDYWGQGTTLTVSS | |
| | VL | QIVLSQSPAILSASPGEKVTMTCRASSSVSYMHWYQQKPGSSPKPW | 84 |
| | | IYATSNLASGVPARFSGSGSGTSYSLTISRVEAEDAATYYCQQWSSN | |
| | | PPTFGGGTKLETK | |
| CD3-4 | VH | QVQLQQSGAELARPGASVKMSCKASGYTFTRYTMHWVKQRPGQG | 79 |
| | | LEWIGYINPSRGYTNYNQKFKDKATLTTDKSSSTAYMQLSSLTSEDS | |
| | | AVYYCARYYDDHYCLDYWGQGTTLTVSS | |
| | VL | QIVLTQSPAIMSASPGEKVTMTCRASSSVSYMNWYQQKSGTSPKR | 85 |
| | | WIYDTSKVASGVPYRFSGSGSGTSYSLTISSMEAEDAATYYCQQWS | |
| | | SNPLTFGSGTKLEIN | |
| CD3-5 | VH | QVQLVQSGGGVVQPGRSLRLSCKASGYTFTRYTMHWVRQAPGKG | 86 |
| | | LEWIGYINPSRGYTNYNQKVKDRFTISRDNSKNTAFLQMDSLRPEDT | |
| | | GVYFCARYYDDHYCLDYWGQGTPVTVSS | |
| | VL | DIQMTQSPSSLSASVGDRVTITCSASSSVSYMNWYQQTPGKAPKR | 87 |
| | | WIYDTSKLASGVPSRFSGSGSGTDYTFTISSLQPEDIATYYCQQWSS | |
| | | NPFTFGQGTKLQIT | |
| CD3-6 | VH | QVQLVESGGGVVQPGRSLRLSCAASGFKFSGYGMHWVRQAPGKG | 88 |
| | | LEWVAVIWYDGSKKYYVDSVKGRFTISRDNSKNTLYLQMNSLRAED | |
| | | TAVYYCARQMGYWHFDLWGRGTLVTVSS | |
| | VL | EIVLTQSPATLSLSPGERATLSCRASQSVSSYLAWYQQKPGQAPRLL | 89 |
| | | IYDASNRATGIPARFSGSGSGTDFTLTISSLEPEDFAVYYCQQRSNW | |
| | | PPLTFGGGTKVEIK | |
| CD3-7 | VH | EVQLVESGGGLVQPGGSLRLSCAASGFTFSTYAMNWVRQAPGKGL | 90 |
| | | EWVGRIRSKYNNYATYYADSVKDRFISRDDSKNSLYLQMNSLKTED | |
| | | TAVYYCVRHGNFGNSYVSWFAYWGQGTLVTVSS | |
| | VL | QAVVTQEPSLTVSPGGTVTLTCRSSTGAVTTSNYANWVQQKPGQA | 91 |
| | | PRGLIGGTNKRAPWTPARFSGSLLGGKAALIGAQAEDEADYYCALW | |
| | | YSNLWVFGGGTKLTVL | |
| CD3-8 | VH | DIKLQQSGAELARPGASVKMSCKTSGYTFTRYTMHWVKQRPGQGL | 92 |
| | | EWIGYINPSRGYTNYNQKFKDKATLTTDKSSSTAYMQLSSLTSEDSA | |
| | | VYYCARYYDDHYCLDYWGQGTTLTVSS | |
| | VL | DIQLTQSPAIMSASPGEKVTMTCRASSSVSYMNWYQQKSGTSPKR | 93 |
| | | WIYDTSKVASGVPYRFSGSGSGTSYSLISSMEAEDAATYYCQQWSS | |
| | | NPLTFGAGTKLELK | |
| CD3-9 | VH | EVQLVESGGGLVQPGGSLRLSCAASGFTFNTYAMNWVRQAPGKGL | 94 |
| | | EWVARIRSKYNNYATYYADSVKDRFISRDDSKNSLYLQMNSLKTEDT | |
| | | AVYYCVRHGNFGNSYVSWFAYWGQGTLVTVSS | |
| | VL | QAVVTQEPSLTVSPGGTVTLTCRSSTGAVTTSNYANWVQQKPGQA | 91 |
| | | PRGLIGGTNKRAPWTPARFSGSLLGGKAALIGAQAEDEADYYCALW | |
| | | YSNLWVFGGGTKLTVL | |
| CD3-10 | VH | EVKLLESGGGLVQPKGSLKLSCAASGFTFNTYAMNWVRQAPGKGL | 95 |
| | | EWVARIRSKYNNYATYYADSVKDRFTISRDDSQSILYLQMNNLKTED | |
| | | TAMYYCVRHGNFGNSYVSWFAYWGQGTLVTVSA | |
| | VL | QAVVTQESALTTSPGETVTLTCRSSTGAVTTSNYANWVQEKPDHLF | 82 |
| | | TGLIGGTNKRAPGVPARFSGSLIGDKAALTITGAQTEDEAIYFCALWY | |
| | | SNLWVFGGGTKLTVL | |
| CD3-11 | VH | EVQLVESGGGLVQPGGSLKLSCAASGFTFNSYAMNWVRQAPGKGL | 96 |
| | | EWVARIRSKYNNYATYYADSVKGRFTISRDDSKNTAYLQMNNLKTE | |
| | | DTAVYYCVRHGNFGNSYVSWWAYWGQGTLVTVSS | |
| | VL | QTVVTQEPSLTVSPGGTVTLTCGSSTGAVTSGNYPNWVQQKPGQA | 97 |
| | | PRGLIGGTKFLAPGTPQRFSGSLLGGKAALTLSGVQPEDEAEYYCVL | |
| | | WYSNRWVFGGGTKLTVL | |
| CD3-12 | VH | EVQLVESGGGLVQPGGSLKLSCAASGFTFNKYAMNWVRQAPGKGL | 98 |
| | | EWVARIRSKYNNYATYYADSVKDRFTISRDDSKNTAYLQMNNLKTE | |
| | | DTAVYYCVRHGNFGNSYISYWAYWGQGTLVTVSS | |
| | VL | QTVVTQEPSLTVSPGGTVTLTCGSSTGAVTSGNYPNWVQQKPGQA | 99 |
| | | PRGLIGGTKFLAPGTPARFSGSLLGGKAALTLSGVQPEDEAEYYCVL | |
| | | WYSNRWVFGGGTKLTVL | |
| CD3-13 | VH | QVQLVQSGAEVKKPGASVKVSCKASGYTFTRYTMHWVRQAPGQGL | 100 |
| | | EWMGYINPSRGYTNYNQKFKDRVTMTTDTSISTAYMELSRLRSDDT | |
| | | AVYYCARYYDDHYCLDYWGQGTLVTVSS | |
| | VL | EIVLTQSPATLSLSPGERATLSCSASSSVSYMNWYQQKPGQAPRLLI | 101 |
| | | YDTSKLASGVPAHFRGSGSGTDFTLTISSLEPEDFAVYYCQQWSSN | |
| | | PFTFGQGTKVEIK | |
| CD3-14 | VH | EVQLVESGGGLVQPGGSLRLSCAASGFTFSTYAMNWVRQAPGKGL | 102 |
| | | EWVSRIRSKYNNYATYYADSVKDRFTISRDDSKNTLYLQMNSLRAED | |
| | | TAVYYCARHGNFGNSYVSWFAYWGQGTMVTVSS | |
| | VL | QAVVTQEPSLTVSPGGTVTLTCRSSTGAVTTSNYANWVQQKPGQA | 103 |
| | | PRGLIGGTNKRAPGTPARFSGSLLGGKAALTLSGVQPEDEAEYYCA | |
| | | LWYSNLWVFGGGTKLTVL | |
| CD3-15 | VH | EVQLVESGGGLVQPGGSLRLSCAASGFTFNTYAMNWVRQAPGKGL | 104 |
| | | EWVGRIRSKYNNYATYYADSVKDRFTISRDDSKNSLYLQMNSLKTE | |
| | | DTAVYYCVRHGNFGNSYVSWFAYWGQGTLVTVSS | |
| | VL | QAVVTQEPSLTVSPGGTVTLTCRSSTGAVTTSNYANWVQEKPGQA | 105 |
| | | PRGLIGGTNKRAPWTPARFSGSLLGGKAALTITGAQAEDEADYYCAL | |
| | | WYSNLWVFGGGTKLTVL | |
| CD3-16 | VH | EVQLVESGGGLVQPGGSLRLSCAASGFTFNTYAMNWVRQAPGKGL | 106 |
| | | EWVGRIRSKYNNYATYYADSVKGRFTISRDDSKNTLYLQMNSLRAE | |
| | | DTAVYYCVRHGNFGNSYVSWFAYWGQGTLVTVSS | |
| | VL | QAVVTQEPSLTVSPGGTVTLTCGSSTGAVTTSNYANWVQQKPGQA | 107 |
| | | PRGLIGGTNKRAPGVPARFSGSLLGGKAALTLSGAQPEDEAEYYCA | |
| | | LWYSNLWVFGGGTKLTVL | |
| CD3-17 | VH | EVQLVESGGGLVQPGGSLRLSCAASGFTFSTYAMNWVRQAPGKGL | 108 |
| | | EWVGRIRSKYNNYATYYADSVKGRFTISRDDSKNTLYLQMNSLRAE | |
| | | DTAVYYCVRHGNFGDSYVSWFAYWGQGTLVTVSS | |
| | VL | QAVVTQEPSLTVSPGGTVTLTCGSSTGAVTTSNYANWVQQKPGKS | 109 |
| | | PRGLIGGTNKRAPGVPARFSGSLLGGKAALTISGAQPEDEADYYCAL | |
| | | WYSNHWVFGGGTKLTVL | |
| CD3-18 | VH | QVQLVQSGGGVVQPGRSLRLSCKASGYTFTRYTMHWVRQAPGKG | 86 |
| | | LEWIGYINPSRGYTNYNQKVKDRFTISRDNSKNTAFLQMDSLRPEDT | |
| | | GVYFCARYYDDHYCLDYWGQGTPVTVSS | |
| | VL | DIQMTQSPSSLSASVGDRVTITCSASSSVSYMNWYQQTPGKAPKR | 110 |
| | | WIYDTSKLASGVPSRFSGSGSGTDYTFTISSLQPEDIATYYCQQWSS | |
| | | NPFTFGQGT | |
| CD3-19 | VH | QVQLVQSGGGVVQPGRSLRLSCKASGYTFTRYTMHWVRQAPGKG | 111 |
| | | LEWIGYINPSRGYTNYNQKVKDRFTISRDNSKNTAFLQMDSLRPEDT | |
| | | GVYFCARYYDDHYSLDYWGQGTPVTVSS | |
| | VL | DIQMTQSPSSLSASVGDRVTITCSASSSVSYMNWYQQTPGKAPKR | 110 |
| | | WIYDTSKLASGVPSRFSGSGSGTDYTFTISSLQPEDIATYYCQQWSS | |
| | | NPFTFGQGT | |
| CD3-20 | VH | EVQLQQSGPELVKPGASMKISCKASGYSFTGYTMNWVKQSHGKNL | 112 |
| | | EWMGLINPYKGVSTYNQKFKDKATLTVDKSSSTAYMELLSLTSEDSA | |
| | | VYYCARSGYYGDSDWYFDVWGQGTTLTVFS | |
| | VL | DIQMTQTTSSLSASLGDRVTISCRASQDIRNYLNWYQQKPDGTVKLL | 113 |
| | | IYYTSRLHSGVPSKFSGSGSGTDYSLTISNLEQEDIATYFCQQGNTLP | |
| | | WTFAGGTKLEIK | |
| CD3-21 | VH | EVQLVESGGGLVQPGGSLKLSCAASGFTFNTYAMNWVRQASGKGL | 114 |
| | | EWVGRIRSKYNNYATYYADSVKDRFTISRDDSKSTLYLQMNSLKTED | |
| | | TAVYYCVRHGNFGNSYVSWFAYWGQGTLVTVSS | |
| | VL | QAVVTQEPSLTVSPGGTVTLTCRSSTGAVTTSNYANWVQQKPGQA | 115 |
| | | PRGLIGGTNKRAPWTPARFSGSLLGDKAALTLSGAQPEDEAEYFCA | |
| | | LWYSNLWVFGGGTKLTVL | |
| | scFv | EVQLVESGGGLVQPGGSLKLSCAASGFTFNTYAMNWVRQASGKGL | 116 |
| | | EWVGRIRSKYNNYATYYADSVKDRFTISRDDSKSTLYLQMNSLKTED | |
| | | TAVYYCVRHGNFGNSYVSWFAYWGQGTLVTVSSGGGGSGGGGSG | |
| | | GGGSGGGGSQAVVTQEPSLTVSPGGTVTLTCRSSTGAVTTSNYAN | |
| | | WVQQKPGQAPRGLIGGTNKRAPWTPARFSGSLLGDKAALTLSGAQ | |
| | | PEDEAEYFCALWYSNLWVFGGGTKLTVL | |
| CD3-22 | VH | EVQLVESGGGLVQPGGSLRLSCAASGFTFSTYAMNWVRQAPGKGL | 108 |
| | | EWVGRIRSKYNNYATYYADSVKGRFTISRDDSKNTLYLQMNSLRAE | |
| | | DTAVYYCVRHGNFGDSYVSWFAYWGQGTLVTVSS | |
| | VL | QAVVTQEPSLTVSPGGTVTLTCGSSTGAVTTSNYANWVQQKPGKS | 109 |
| | | PRGLIGGTNKRAPGVPARFSGSLLGGKAALTISGAQPEDEADYYCAL | |
| | | WYSNHWVFGGGTKLTVL | |
| | scFv | EVQLVESGGGLVQPGGSLRLSCAASGFTFSTYAMNWVRQAPGKGL | 117 |
| | | EWVGRIRSKYNNYATYYADSVKGRFTISRDDSKNTLYLQMNSLRAE | |
| | | DTAVYYCVRHGNFGDSYVSWFAYWGQGTLVTVSSGKPGSGKPGS | |
| | | GKPGSGKPGSQAVVTQEPSLTVSPGGTVTLTCGSSTGAVTTSNYAN | |
| | | WVQQKPGKSPRGLIGGTNKRAPGVPARFSGSLLGGKAALTISGAQP | |
| | | EDEADYYCALWYSNHWVFGGGTKLTVL | |
| CD3-23 | VH | EVQLVESGGGLVQPGGSLRLSCAASGFTFSTYAMNWVRQAPGKGL | 118 |
| | | EWVGRIRSKANNYATYYADSVKGRFTISRDDSKNTLYLQMNSLRAE | |
| | | DTAVYYCVRHGNFGDSYVSWFAYWGQGTLVTVSS | |
| | VL | QAVVTQEPSLTVSPGGTVTLTCGSSTGAVTTSNYANWVQQKPGKS | 109 |
| | | PRGLIGGTNKRAPGVPARFSGSLLGGKAALTISGAQPEDEADYYCAL | |
| | | WYSNHWVFGGGTKLTVL | |
| | scFv | EVQLVESGGGLVQPGGSLRLSCAASGFTFSTYAMNWVRQAPGKGL | 119 |
| | | EWVGRIRSKANNYATYYADSVKGRFTISRDDSKNTLYLQMNSLRAE | |
| | | DTAVYYCVRHGNFGDSYVSWFAYWGQGTLVTVSSGKPGSGKPGS | |
| | | GKPGSGKPGSQAVVTQEPSLTVSPGGTVTLTCGSSTGAVTTSNYAN | |
| | | WVQQKPGKSPRGLIGGTNKRAPGVPARFSGSLLGGKAALTISGAQP | |
| | | EDEADYYCALWYSNHWVFGGGTKLTVL | |
| CD3-24 | VH | EVQLVESGGGLVQPGGSLRLSCAASGFTFSTYAMNWVRQAPGKGL | 120 |
| | | EWVGRIRSKYNNYATYYADSVKGRFTISRDDSKNTLYLQMNSLRAE | |
| | | DTAVYYCVRHGNFGDEYVSWFAYWGQGTLVTVSS | |
| | VL | QAVVTQEPSLTVSPGGTVTLTCGSSTGAVTTSNYANWVQQKPGKS | 109 |
| | | PRGLIGGTNKRAPGVPARFSGSLLGGKAALTISGAQPEDEADYYCAL | |
| | | WYSNHWVFGGGTKLTVL | |
| | scFv | EVQLVESGGGLVQPGGSLRLSCAASGFTFSTYAMNWVRQAPGKGL | 121 |
| | | EWVGRIRSKYNNYATYYADSVKGRFTISRDDSKNTLYLQMNSLRAE | |
| | | DTAVYYCVRHGNFGDEYVSWFAYWGQGTLVTVSSGKPGSGKPGS | |
| | | GKPGSGKPGSQAVVTQEPSLTVSPGGTVTLTCGSSTGAVTTSNYAN | |
| | | WVQQKPGKSPRGLIGGTNKRAPGVPARFSGSLLGGKAALTISGAQP | |
| | | EDEADYYCALWYSNHWVFGGGTKLTVL | |
| CD3-25 | VH | EVQLVESGGGLVQPGGSLRLSCAASGFTFSTYAMNWVRQAPGKGL | 122 |
| | | EWVGRIRSKYNNYATYYADSVKGRFTISRDDSKNTLYLQMNSLRAE | |
| | | DTAVYYCVRHGNFGDPYVSWFAYWGQGTLVTVSS | |
| | VL | QAVVTQEPSLTVSPGGTVTLTCGSSTGAVTTSNYANWVQQKPGKS | 109 |
| | | PRGLIGGTNKRAPGVPARFSGSLLGGKAALTISGAQPEDEADYYCAL | |
| | | WYSNHWVFGGGTKLTVL | |
| | scFv | EVQLVESGGGLVQPGGSLRLSCAASGFTFSTYAMNWVRQAPGKGL | 123 |
| | | EWVGRIRSKYNNYATYYADSVKGRFTISRDDSKNTLYLQMNSLRAE | |
| | | DTAVYYCVRHGNFGDPYVSWFAYWGQGTLVTVSSGKPGSGKPGS | |
| | | GKPGSGKPGSQAVVTQEPSLTVSPGGTVTLTCGSSTGAVTTSNYAN | |
| | | WVQQKPGKSPRGLIGGTNKRAPGVPARFSGSLLGGKAALTISGAQP | |
| | | EDEADYYCALWYSNHWVFGGGTKLTVL | |
| CD3-26 | VH | EVQLVESGGGLVQPGGSLRLSCAASGFTFSTYAMNWVRQAPGKGL | 124 |
| | | EWVGRIRSKYNNYATYYADSVKGRFTISRDDSKNTLYLQMNSLRAE | |
| | | DTAVYYCVRHGNFGDSYVSWFDYWGQGTLVTVSS | |
| | VL | QAVVTQEPSLTVSPGGTVTLTCGSSTGAVTTSNYANWVQQKPGKS | 109 |
| | | PRGLIGGTNKRAPGVPARFSGSLLGGKAALTISGAQPEDEADYYCAL | |
| | | WYSNHWVFGGGTKLTVL | |
| | scFv | EVQLVESGGGLVQPGGSLRLSCAASGFTFSTYAMNWVRQAPGKGL | 125 |
| | | EWVGRIRSKYNNYATYYADSVKGRFTISRDDSKNTLYLQMNSLRAE | |
| | | DTAVYYCVRHGNFGDSYVSWFDYWGQGTLVTVSSGKPGSGKPGS | |
| | | GKPGSGKPGSQAVVTQEPSLTVSPGGTVTLTCGSSTGAVTTSNYAN | |
| | | WVQQKPGKSPRGLIGGTNKRAPGVPARFSGSLLGGKAALTISGAQP | |
| | | EDEADYYCALWYSNHWVFGGGTKLTVL | |
| CD3-27 | VH | EVQLVESGGGLVQPGGSLRLSCAASGFTFSTYAMSWVRQAPGKGL | 126 |
| | | EWVGRIRSKYNNYATYYADSVKGRFTISRDDSKNTLYLQMNSLRAE | |
| | | DTAVYYCVRHGNFGDSYVSWFAYWGQGTLVTVSS | |
| | VL | QAVVTQEPSLTVSPGGTVTLTCGSSTGAVTTSNYANWVQQKPGKS | 109 |
| | | PRGLIGGTNKRAPGVPARFSGSLLGGKAALTISGAQPEDEADYYCAL | |
| | | WYSNHWVFGGGTKLTVL | |
| | scFv | EVQLVESGGGLVQPGGSLRLSCAASGFTFSTYAMSWVRQAPGKGL | 127 |
| | | EWVGRIRSKYNNYATYYADSVKGRFTISRDDSKNTLYLQMNSLRAE | |
| | | DTAVYYCVRHGNFGDSYVSWFAYWGQGTLVTVSSGKPGSGKPGS | |
| | | GKPGSGKPGSQAVVTQEPSLTVSPGGTVTLTCGSSTGAVTTSNYAN | |
| | | WVQQKPGKSPRGLIGGTNKRAPGVPARFSGSLLGGKAALTISGAQP | |
| | | EDEADYYCALWYSNHWVFGGGTKLTVL | |
| CD3-28 | VH | EVQLVESGGGLVQPGGSLRLSCAASGFTFNTYAMNWVRQAPGKGL | 106 |
| | | EWVGRIRSKYNNYATYYADSVKGRFTISRDDSKNTLYLQMNSLRAE | |
| | | DTAVYYCVRHGNFGNSYVSWFAYWGQGTLVTVSS | |
| | VL | QAVVTQEPSLTVSPGGTVTLTCGSSTGAVTTSNYANWVQQKPGQA | 107 |
| | | PRGLIGGTNKRAPGVPARFSGSLLGGKAALTLSGAQPEDEAEYYCA | |
| | | LWYSNLWVFGGGTKLTVL | |
| | scFv | EVQLVESGGGLVQPGGSLRLSCAASGFTFNTYAMNWVRQAPGKGL | 128 |
| | | EWVGRIRSKYNNYATYYADSVKGRFTISRDDSKNTLYLQMNSLRAE | |
| | | DTAVYYCVRHGNFGNSYVSWFAYWGQGTLVTVSSGGGGSGGGGS | |
| | | GGGGSQAVVTQEPSLTVSPGGTVTLTCGSSTGAVTTSNYANWVQQ | |
| | | KPGQAPRGLIGGTNKRAPGVPARFSGSLLGGKAALTLSGAQPEDEA | |
| | | EYYCALWYSNLWVFGGGTKLTVLGSHHHHHH | |

CDR sequences for a number of CD3 binders as defined by the Kabat numbering scheme (Kabat et al, 1991, Sequences of Proteins of Immunological Interest, 5^{th} Ed. Public Health Service, National Institutes of Health, Bethesda, Md.), Chothia numbering scheme (Al-Lazikani et al., 1997, J. Mol. Biol 273:927-948), and a combination of Kabat and Chothia numbering are provided in Tables 7B-7D, respectively.

TABLE 7B
CD3 Binders-CDR sequences according to Kabat numbering scheme

| Binding | | | SEQ ID | | SEQ ID | | SEQ ID |
|---|---|---|---|---|---|---|---|
| Domain | Chain | CDR1 | NO: | CDR2 | NO: | CDR3 | NO: |
| CD3-1 | VH | RYTMH | 129 | YINPSRGYTNYNQK | 149 | YYDDHYCLDY | 173 |
| | | | | FKD | | | |
| | VL | SASSSVSYM | 130 | DTSKLAS | 150 | QQWSSNPFT | 174 |
| | | N | | | | | |
| CD3-2 | VH | TYAMN | 131 | RIRSKYNNYATYYA | 151 | HGNFGNSYVSW | 175 |
| | | | | DSVKD | | FAY | |
| | VL | RSSTGAVTT | 132 | GTNKRAP | 152 | ALWYSNLWV | 176 |
| | | SNYAN | | | | | |
| CD3-3 | VH | SYTMH | 133 | YINPSSGYTKYNQK | 153 | WQDYDVYFDY | 177 |
| | | | | FKD | | | |
| | VL | RASSSVSYM | 134 | ATSNLAS | 154 | QQWSSNPPT | 178 |
| | | H | | | | | |
| CD3-4 | VH | RYTMH | 129 | YINPSRGYTNYNQK | 149 | YYDDHYCLDY | 173 |
| | | | | FKD | | | |
| | VL | RASSSVSYM | 135 | DTSKVAS | 155 | QQWSSNPLT | 179 |
| | | N | | | | | |
| CD3-5 | VH | RYTMH | 129 | YINPSRGYTNYNQK | 156 | YYDDHYCLDY | 173 |
| | | | | VKD | | | |
| | VL | SASSSVSYM | 130 | DTSKLAS | 150 | QQWSSNPFT | 174 |
| | | N | | | | | |
| CD3-6 | VH | GYGMH | 136 | VIWYDGSKKYYVDS | 157 | QMGYWHFDL | 180 |
| | | | | VKG | | | |
| | VL | RASQSVSSY | 137 | DASNRAT | 158 | QQRSNWPPLT | 181 |
| | | LA | | | | | |
| CD3-7 | VH | TYAMN | 131 | RIRSKYNNYATYYA | 159 | VRHGNFGNSYV | 182 |
| | | | | D | | SWFAY | |
| | VL | RSSTGAVTT | 132 | GTNKRAP | 152 | ALWYSNLWV | 176 |
| | | SNYAN | | | | | |
| CD3-8 | VH | RYTMH | 129 | YINPSRGYTNYNQK | 149 | YYDDHYCLDY | 173 |
| | | | | FKD | | | |
| | VL | RASSSVSYM | 135 | DTSKVAS | 155 | QQWSSNPLT | 179 |
| | | N | | | | | |
| CD3-9 | VH | TYAMN | 131 | RIRSKYNNYATYYA | 159 | VRHGNFGNSYV | 182 |
| | | | | D | | SWFAY | |
| | VL | RSSTGAVTT | 132 | GTNKRAP | 152 | ALWYSNLWV | 176 |
| | | SNYAN | | | | | |
| CD3-10 | VH | TYAMN | 131 | RIRSKYNNYATYYA | 151 | HGNFGNSYVSW | 175 |
| | | | | DSVKD | | FAY | |
| | VL | RSSTGAVTT | 132 | GTNKRAP | 152 | ALWYSNLWV | 176 |
| | | SNYAN | | | | | |
| CD3-11 | VH | SYAMN | 138 | RIRSKYNNYATYYA | 160 | HGNFGNSYVSW | 183 |
| | | | | DSVKG | | WAY | |
| | VL | GSSTGAVTS | 139 | GTKFLAP | 161 | VLWYSNRWV | 184 |
| | | GNYPN | | | | | |
| CD3-12 | VH | KYAMN | 140 | RIRSKYNNYATYYA | 151 | HGNFGNSYISY | 185 |
| | | | | DSVKD | | WAY | |
| | VL | GSSTGAVTS | 139 | GTKFLAP | 161 | VLWYSNRWV | 184 |
| | | GNYPN | | | | | |
| CD3-13 | VH | RYTMH | 129 | YINPSRGYTNYNQK | 149 | YYDDHYCLDY | 173 |
| | | | | FKD | | | |
| | VL | SASSSVSYM | 130 | DTSKLAS | 150 | QQWSSNPFT | 174 |
| | | N | | | | | |
| CD3-14 | VH | TYAMN | 131 | RIRSKYNNYATYYA | 151 | HGNFGNSYVSW | 175 |
| | | | | DSVKD | | FAY | |
| | VL | RSSTGAVTT | 132 | GTNKRAP | 152 | ALWYSNLWV | 176 |
| | | SNYAN | | | | | |
| CD3-15 | VH | TYAMN | 131 | RIRSKYNNYATYYA | 151 | HGNFGNSYVSW | 175 |
| | | | | DSVKD | | FAY | |
| | VL | RSSTGAVTT | 132 | GTNKRAP | 152 | ALWYSNLWV | 176 |
| | | SNYAN | | | | | |
| CD3-16 | VH | TYAMN | 131 | RIRSKYNNYATYYA | 160 | HGNFGNSYVSW | 175 |
| | | | | DSVKG | | FAY | |
| | VL | GSSTGAVTT | 141 | GTNKRAP | 152 | ALWYSNLWV | 176 |
| | | SNYAN | | | | | |
| CD3-17 | VH | TYAMN | 131 | RIRSKYNNYATYYA | 160 | HGNFGDSYVSW | 186 |
| | | | | DSVKG | | FAY | |
| | VL | GSSTGAVTT | 141 | GTNKRAP | 152 | ALWYSNHWV | 187 |
| | | SNYAN | | | | | |
| CD3-18 | VH | RYTMH | 129 | YINPSRGYTNYNQK | 156 | YYDDHYCLDY | 173 |
| | | | | VKD | | | |
| | VL | SASSSVSYM | 130 | DTSKLAS | 150 | QQWSSNPFT | 174 |
| | | N | | | | | |
| CD3-19 | VH | RYTMH | 129 | YINPSRGYTNYNQK | 156 | YYDDHYSLDY | 188 |
| | | | | VKD | | | |
| | VL | SASSSVSYM | 130 | DTSKLAS | 150 | QQWSSNPFT | 174 |
| | | N | | | | | |
| CD3-20 | VH | GYTMN | 142 | LINPYKGVSTYNQK | 162 | SGYYGDSDWYF | 189 |
| | | | | FKD | | DV | |
| | VL | RASQDIRNY | 143 | YTSRLH | 163 | QQGNTLPWT | 190 |
| | | LN | | | | | |
| CD3-21 | VH | TYAMN | 131 | RIRSKYNNYATYYA | 151 | HGNFGNSYVSW | 175 |
| | | | | DSVKD | | FAY | |
| | VL | RSSTGAVTT | 132 | GTNKRAP | 152 | ALWYSNLWV | 176 |
| | | SNYAN | | | | | |
| CD3-22 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-23 | VH | TYAMN | 131 | RIRSKANNYATYY | 164 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-24 | VH | TYAMN | 131 | RIRSKYNNYATYY | 160 | HGNFGDEYVS | 191 |
| | | | | ADSVKG | | WFAY | |
| | VL | GSSTGAVT | 141 | GTNKRAP | 152 | ALWYSNHWV | 187 |
| | | TSNYAN | | | | | |
| CD3-25 | VH | TYAMN | 131 | RIRSKYNNYATYY | 160 | HGNFGDPYVS | 192 |
| | | | | ADSVKG | | WFAY | |
| | VL | GSSTGAVT | 141 | GTNKRAP | 152 | ALWYSNHWV | 187 |
| | | TSNYAN | | | | | |
| CD3-26 | VH | TYAMN | 131 | RIRSKYNNYATYY | 160 | HGNFGDSYVS | 193 |
| | | | | ADSVKG | | WFDY | |
| | VL | GSSTGAVT | 141 | GTNKRAP | 152 | ALWYSNHWV | 187 |
| | | TSNYAN | | | | | |
| CD3-27 | VH | TYAMS | 144 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-28 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-29 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-30 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-31 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-32 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-33 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-34 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-35 | VH | TYAMH | 145 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-36 | VH | TYAMS | 144 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-37 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-38 | VH | TYAMN | 131 | RIRSKANNYYATY | 165 | HGNFGNSYVS | 175 |
| | | | | YADSVKG | | WFAY | |
| | VL | GSSTGAVT | 141 | GTNKRAP | 152 | ALWYSNLWV | 176 |
| | | TSNYAN | | | | | |
| CD3-39 | VH | TYAMN | 131 | RIRSKANSYATYY | 166 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-40 | VH | TYAMN | 131 | RIRSKYNNYATAY | 167 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-41 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-42 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-43 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-44 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-45 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-46 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-47 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-48 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-49 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-50 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNLWV | 176 |
| | | TSNYAN | | | | | |
| CD3-51 | VH | TYAMN | 131 | RIRSKYNNYATYY | 160 | HGNFGQSYVS | 194 |
| | | | | ADSVKG | | WFAY | |
| | VL | GSSTGAVT | 141 | GTNKRAP | 152 | ALWYSNLWV | 176 |
| | | TSNYAN | | | | | |
| CD3-52 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-53 | VH | TYAMN | 131 | RIRSKYNNYATYY | 160 | HGNFGNSYVS | 195 |
| | | | | ADSVKG | | WFDY | |
| | VL | GSSTGAVT | 141 | GTNKRAP | 152 | ALWYSNLWV | 176 |
| | | TSNYAN | | | | | |
| CD3-54 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-55 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-56 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-57 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-58 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-59 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-60 | VH | TYAMN | 131 | RIRSKYNNYATYY | 160 | HGNFGNSYVS | 175 |
| | | | | ADSVKG | | WFAY | |
| | VL | GSSTGAVT | 146 | GTNKRAP | 152 | ALWYSNLWV | 176 |
| | | SSNYAN | | | | | |
| CD3-61 | VH | TYAMN | 131 | RIRSKYNNYATYY | 160 | HGNFGNSYVS | 175 |
| | | | | ADSVKG | | WFAY | |
| | VL | GSSTGAVT | 147 | GTNKRAP | 152 | ALWYSNLWV | 176 |
| | | SGHYAN | | | | | |
| CD3-62 | VH | TYAMN | 131 | RIRSKYNNYATYY | 160 | HGNFGNSYVS | 175 |
| | | | | ADSVKG | | WFAY | |
| | VL | GSSTGAVT | 141 | DTNKRAP | 168 | ALWYSNLWV | 176 |
| | | TSNYAN | | | | | |
| CD3-63 | VH | TYAMN | 131 | RIRSKYNNYATYY | 160 | HGNFGNSYVS | 175 |
| | | | | ADSVKG | | WFAY | |
| | VL | GSSTGAVT | 141 | GTNNRAP | 169 | ALWYSNLWV | 176 |
| | | TSNYAN | | | | | |
| CD3-64 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAS | 170 | ALWYSNLWV | 176 |
| | | TSNYAN | | | | | |
| CD3-65 | VH | TYAMN | 131 | RIRSKYNNYATYY | 160 | HGNFGNSYVS | 175 |
| | | | | ADSVKG | | WFAY | |
| | VL | GSSTGAVT | 141 | GTSNKHS | 171 | ALWYSNLWV | 176 |
| | | TSNYAN | | | | | |
| CD3-66 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-67 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-68 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-69 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-70 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-71 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-72 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-73 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | LLWYSNLWV | 196 |
| | | TSNYAN | | | | | |
| CD3-74 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-75 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-76 | VH | TYAMN | 131 | RIRSKYNNYATYY | 160 | HGNFGNSYVS | 175 |
| | | | | ADSVKG | | WFAY | |
| | VL | RSSTGAVT | 132 | GTNKRAP | 152 | ALWYSNLWV | 176 |
| | | TSNYAN | | | | | |
| CD3-77 | VH | TYAMN | 131 | RIRSKYNNYATYY | 160 | HGNFGNSYVS | 175 |
| | | | | ADSVKG | | WFAY | |
| | VL | KSSTGAVT | 148 | GTNKRAP | 152 | ALWYSNLWV | 176 |
| | | TSNYAN | | | | | |
| CD3-78 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-79 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-80 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-81 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-82 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-83 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-84 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-85 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-86 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-87 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNLWV | 176 |
| | | TSNYAN | | | | | |
| CD3-88 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNLWV | 176 |
| | | TSNYAN | | | | | |
| CD3-89 | VH | TYAMN | 131 | RIRSKANNYATYY | 164 | HGNFGDSYVS | 186 |
| | | | | ADSVKG | | WFAY | |
| | VL | GSSTGAVT | 141 | GTNKRAP | 152 | ALWYSNLWV | 176 |
| | | TSNYAN | | | | | |
| CD3-90 | VH | TYAMN | 131 | RIRSKYNNYATYY | 160 | HGNFGDSYVS | 193 |
| | | | | ADSVKG | | WFDY | |
| | VL | GSSTGAVT | 141 | GTNKRAP | 152 | ALWYSNLWV | 176 |
| | | TSNYAN | | | | | |
| CD3-91 | VH | TYAMS | 144 | RIRSKANNYATYY | 164 | HGNFGDSYVS | 193 |
| | | | | ADSVKG | | WFDY | |
| | VL | GSSTGAVT | 141 | GTNKRAP | 152 | ALWYSNLWV | 176 |
| | | TSNYAN | | | | | |
| CD3-92 | VH | TYAMN | 131 | RIRSNGGYSTYYA | 172 | HGNFGNSYVS | 175 |
| | | | | DSVKG | | WFAY | |
| | VL | GSSTGAVT | 141 | GTNKRAP | 152 | ALWYSNLWV | 176 |
| | | TSNYAN | | | | | |
| CD3-93 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-94 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-95 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNLWV | 176 |
| | | TSNYAN | | | | | |
| CD3-96 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-97 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-98 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-99 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-100 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-101 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-102 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-103 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-104 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-105 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-106 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-107 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-108 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-109 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-110 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-111 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-112 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-113 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-114 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-115 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-116 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-117 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-118 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-119 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-120 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-121 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-122 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-123 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-124 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-125 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |
| CD3-126 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-127 | VH | TYAMN | 131 | RIRSKYNNYATYY | 160 | HGNFGNSYVS | 175 |
| | | | | ADSVKG | | WFAY | |
| | VL | GSSTGAVT | 141 | GTNKRAP | 152 | ALWYSNHWV | 187 |
| | | TSNYAN | | | | | |
| CD3-128 | VH | TYAMN | 131 | RIRSKYNNYATYY | 160 | HGNFGDSYVS | 186 |
| | | | | ADSVKG | | WFAY | |
| | VL | GSSTGAVT | 141 | GTNKRAP | 152 | ALWYSNHWV | 187 |
| | | TSNYAN | | | | | |

TABLE 7C
CD3 Binders - CDR sequences according to
Chothia numbering scheme

| | | | SEQ | | SEQ | | SEQ |
|---|---|---|---|---|---|---|---|
| Binding | | | ID | | ID | | ID |
| Domain | Chain | CDR1 | NO: | CDR2 | NO: | CDR3 | NO: |
| CD3-1 | VH | GYTFTRY | 197 | NPSRGY | 210 | YYDDHYCLDY | 173 |
| | VL | SSSVSY | 198 | DTS | 211 | WSSNPF | 222 |
| CD3-2 | VH | GFTFNTY | 199 | RSKYNN | 212 | HGNFGNSYVS | 175 |
| | | | | YA | | WFAY | |
| | VL | STGAVTTSNY | 200 | GTN | 213 | WYSNLW | 223 |
| CD3-3 | VH | GYTFTSY | 201 | NPSSGY | 214 | WQDYDVYFDY | 177 |
| | VL | SSSVSY | 198 | ATS | 215 | WSSNPP | 224 |
| CD3-4 | VH | GYTFTRY | 197 | NPSRGY | 210 | YYDDHYCLDY | 173 |
| | VL | SSSVSY | 198 | DTS | 211 | WSSNPL | 225 |
| CD3-5 | VH | GYTFTRY | 197 | NPSRGY | 210 | YYDDHYCLDY | 173 |
| | VL | SSSVSY | 198 | DTS | 211 | WSSNPF | 222 |
| CD3-6 | VH | GFKFSGY | 202 | WYDGSK | 216 | QMGYWHFDL | 180 |
| | VL | SQSVSSY | 203 | DAS | 217 | RSNWPPL | 226 |
| CD3-7 | VH | GFTFSTY | 204 | RSKYNN | 218 | HGNFGNSYVS | 227 |
| | | | | YAT | | WFA | |
| | VL | STGAVTTSNY | 200 | GTN | 213 | WYSNLW | 223 |
| CD3-8 | VH | GYTFTRY | 197 | NPSRGY | 210 | YYDDHYCLDY | 173 |
| | VL | SSSVSY | 198 | DTS | 211 | WSSNPL | 225 |
| CD3-9 | VH | GFTFNTY | 199 | RSKYNN | 218 | HGNFGNSYVS | 227 |
| | | | | YAT | | WFA | |
| | VL | STGAVTTSNY | 200 | GTN | 213 | WYSNLW | 223 |
| CD3-10 | VH | GFTFNTY | 199 | RSKYNN | 212 | HGNFGNSYVS | 175 |
| | | | | YA | | WFAY | |
| | VL | STGAVTTSNY | 200 | GTN | 213 | WYSNLW | 223 |
| CD3-11 | VH | GFTFNSY | 205 | RSKYNN | 212 | HGNFGNSYVS | 183 |
| | | | | YA | | WWAY | |
| | VL | STGAVTSGNY | 206 | GTK | 219 | WYSNRW | 228 |
| CD3-12 | VH | GFTFNKY | 207 | RSKYNN | 212 | HGNFGNSYISY | 185 |
| | | | | YA | | WAY | |
| | VL | STGAVTSGNY | 206 | GTK | 219 | WYSNRW | 228 |
| CD3-13 | VH | GYTFTRY | 197 | NPSRGY | 210 | YYDDHYCLDY | 173 |
| | VL | SSSVSY | 198 | DTS | 211 | WSSNPF | 222 |
| CD3-14 | VH | GFTFSTY | 204 | RSKYNN | 212 | HGNFGNSYVS | 175 |
| | | | | YA | | WFAY | |
| | VL | STGAVTTSNY | 200 | GTN | 213 | WYSNLW | 223 |
| CD3-15 | VH | GFTFNTY | 199 | RSKYNN | 212 | HGNFGNSYVS | 175 |
| | | | | YA | | WFAY | |
| | VL | STGAVTTSNY | 200 | GTN | 213 | WYSNLW | 223 |
| CD3-16 | VH | GFTFNTY | 199 | RSKYNN | 212 | HGNFGNSYVS | 175 |
| | | | | YA | | WFAY | |
| | VL | STGAVTTSNY | 200 | GTN | 213 | WYSNLW | 223 |
| CD3-17 | VH | GFTFSTY | 204 | RSKYNN | 212 | HGNFGDSYVS | 186 |
| | | | | YA | | WFAY | |
| | VL | STGAVTTSNY | 200 | GTN | 213 | WYSNHW | 229 |
| CD3-18 | VH | GYTFTRY | 197 | NPSRGY | 210 | YYDDHYCLDY | 173 |
| | VL | SSSVSY | 198 | DTS | 211 | WSSNPF | 222 |
| CD3-19 | VH | GYTFTRY | 197 | NPSRGY | 210 | YYDDHYSLDY | 188 |
| | VL | SSSVSY | 198 | DTS | 211 | WSSNPF | 222 |
| CD3-20 | VH | GYSFTGY | 208 | NPYKGV | 220 | SGYYGDSDWY | 189 |
| | | | | | | FDV | |
| | VL | SQDIRNY | 209 | YTS | 221 | GNTLPW | 230 |
| CD3-21 | VH | GFTFNTY | 199 | RSKYNN | 212 | HGNFGNSYVS | 175 |
| | | | | YA | | WFAY | |
| | VL | RSSTGAVTTS | 132 | GTNKRA | 152 | ALWYSNLWV | 176 |
| | | NYAN | | P | | | |

TABLE 7D
CD3 Binders - CDR sequences according to
combination of Kabat and Chothia
numbering schemes

| | | | SEQ | | SEQ | | SEQ |
|---|---|---|---|---|---|---|---|
| Binding | | | ID | | ID | | ID |
| Domain | Chain | CDR1 | NO: | CDR2 | NO: | CDR3 | NO: |
| CD3-1 | VH | GYTFTRYTM | 231 | YINPSRGYT | 149 | YYDDHYCL | 173 |
| | | H | | NYNQKFKD | | DY | |
| | VL | SASSSVSYM | 130 | DTSKLAS | 150 | QQWSSNPF | 174 |
| | | N | | | | T | |
| CD3-2 | VH | GFTFNTYAM | 232 | RIRSKYNNY | 151 | HGNFGNSY | 175 |
| | | N | | ATYYADSVK | | VSWFAY | |
| | | | | D | | | |
| | VL | RSSTGAVTT | 132 | GTNKRAP | 152 | ALWYSNLW | 176 |
| | | SNYAN | | | | V | |
| CD3-3 | VH | GYTFTSYTM | 233 | YINPSSGYT | 153 | WQDYDVYF | 177 |
| | | H | | KYNQKFKD | | DY | |
| | VL | RASSSVSYM | 134 | ATSNLAS | 154 | QQWSSNPP | 178 |
| | | H | | | | T | |
| CD3-4 | VH | GYTFTRYTM | 231 | YINPSRGYT | 149 | YYDDHYCL | 173 |
| | | H | | NYNQKFKD | | DY | |
| | VL | RASSSVSYM | 135 | DTSKVAS | 155 | QQWSSNPL | 179 |
| | | N | | | | T | |
| CD3-5 | VH | GYTFTRYTM | 231 | YINPSRGYT | 156 | YYDDHYCL | 173 |
| | | H | | NYNQKVKD | | DY | |
| | VL | SASSSVSYM | 130 | DTSKLAS | 150 | QQWSSNPF | 174 |
| | | N | | | | T | |
| CD3-6 | VH | GFKFSGYGM | 234 | VIWYDGSKK | 157 | QMGYWHFD | 180 |
| | | H | | YYVDSVKG | | L | |
| | VL | RASQSVSSY | 137 | DASNRAT | 158 | QQRSNWPP | 181 |
| | | LA | | | | LT | |
| CD3-7 | VH | GFTFSTYAM | 235 | RIRSKYNNY | 239 | HGNFGNSY | 175 |
| | | N | | ATYYADSVK | | VSWFAY | |
| | VL | RSSTGAVTT | 132 | GTNKRAP | 152 | ALWYSNLW | 176 |
| | | SNYAN | | | | V | |
| CD3-8 | VH | GYTFTRYTM | 231 | YINPSRGYT | 149 | YYDDHYCL | 173 |
| | | H | | NYNQKFKD | | DY | |
| | VL | RASSSVSYM | 135 | DTSKVAS | 155 | QQWSSNPL | 179 |
| | | N | | | | T | |
| CD3-9 | VH | GFTFNTYAM | 232 | RIRSKYNNY | 239 | HGNFGNSY | 175 |
| | | N | | ATYYADSVK | | VSWFAY | |
| | VL | RSSTGAVTT | 132 | GTNKRAP | 152 | ALWYSNLW | 176 |
| | | SNYAN | | | | V | |
| CD3-10 | VH | GFTFNTYAM | 232 | RIRSKYNNY | 151 | HGNFGNSY | 175 |
| | | N | | ATYYADSVK | | VSWFAY | |
| | | | | D | | | |
| | VL | RSSTGAVTT | 132 | GTNKRAP | 152 | ALWYSNLW | 176 |
| | | SNYAN | | | | V | |
| CD3-11 | VH | GFTFNSYAM | 236 | RIRSKYNNY | 160 | HGNFGNSY | 183 |
| | | N | | ATYYADSVK | | VSWWAY | |
| | | | | G | | | |
| | VL | GSSTGAVTS | 139 | GTKFLAP | 161 | VLWYSNRW | 184 |
| | | GNYPN | | | | V | |
| CD3-12 | VH | GFTFNKYAM | 237 | RIRSKYNNY | 151 | HGNFGNSY | 185 |
| | | N | | ATYYADSVK | | ISYWAY | |
| | | | | D | | | |
| | VL | GSSTGAVTS | 139 | GTKFLAP | 161 | VLWYSNRW | 184 |
| | | GNYPN | | | | V | |
| CD3-13 | VH | GYTFTRYTM | 231 | YINPSRGYT | 149 | YYDDHYCL | 173 |
| | | H | | NYNQKFKD | | DY | |
| | VL | SASSSVSYM | 130 | DTSKLAS | 150 | QQWSSNPF | 174 |
| | | N | | | | T | |
| CD3-14 | VH | GFTFSTYAM | 235 | RIRSKYNNY | 151 | HGNFGNSY | 175 |
| | | N | | ATYYADSVK | | VSWFAY | |
| | | | | D | | | |
| | VL | RSSTGAVTT | 132 | GTNKRAP | 152 | ALWYSNLW | 176 |
| | | SNYAN | | | | V | |
| CD3-15 | VH | GFTFNTYAM | 232 | RIRSKYNNY | 151 | HGNFGNSY | 175 |
| | | N | | ATYYADSVK | | VSWFAY | |
| | | | | D | | | |
| | VL | RSSTGAVTT | 132 | GTNKRAP | 152 | ALWYSNLW | 176 |
| | | SNYAN | | | | V | |
| CD3-16 | VH | GFTFNTYAM | 232 | RIRSKYNNY | 160 | HGNFGNSY | 175 |
| | | N | | ATYYADSVK | | VSWFAY | |
| | | | | G | | | |
| | VL | GSSTGAVTT | 141 | GTNKRAP | 152 | ALWYSNLW | 176 |
| | | SNYAN | | | | V | |
| CD3-17 | VH | GFTFSTYAM | 235 | RIRSKYNNY | 160 | HGNFGDSY | 186 |
| | | N | | ATYYADSVK | | VSWFAY | |
| | | | | G | | | |
| | VL | GSSTGAVTT | 141 | GTNKRAP | 152 | ALWYSNHW | 187 |
| | | SNYAN | | | | V | |
| CD3-18 | VH | GYTFTRYTM | 231 | YINPSRGYT | 156 | YYDDHYCL | 173 |
| | | H | | NYNQKVKD | | DY | |
| | VL | SASSSVSYM | 130 | DTSKLAS | 150 | QQWSSNPF | 174 |
| | | N | | | | T | |
| CD3-19 | VH | GYTFTRYTM | 231 | YINPSRGYT | 156 | YYDDHYSL | 188 |
| | | H | | NYNQKVKD | | DY | |
| | VL | SASSSVSYM | 130 | DTSKLAS | 150 | QQWSSNPF | 174 |
| | | N | | | | T | |
| CD3-20 | VH | GYSFTGYTM | 238 | LINPYKGVS | 162 | SGYYGDSD | 189 |
| | | N | | TYNQKFKD | | WYFDV | |
| | VL | RASQDIRNYL | 143 | YTSRLHS | 240 | QQGNTLPW | 190 |
| | | N | | | | T | |

In some embodiments, a TBM of the disclosure can comprise a CD3 ABM which comprises the CDRs of any of CD3-1 to CD3-128 as defined by Kabat numbering (e.g., as set forth in Table 7B). In other embodiments, a TBM of the disclosure can comprise a CD3 ABM which comprises the CDRs of any of CD3-1 to CD3-128 as defined by Chothia numbering (e.g., as set forth in Table 7C). In yet other embodiments, a TBM of the disclosure can comprise a CD3 ABM which comprises the CDRs of any of CD3-1 to CD3-128 as defined by a combination of Kabat and Chothia numbering (e.g., as set forth in Table 7D).

In some embodiments, a CD3 ABM comprises the CDR sequences of CD3-1. In some embodiments, a CD3 ABM comprises the CDR sequences of CD3-2. In some embodiments, a CD3 ABM comprises the CDR sequences of CD3-3. In some embodiments, a CD3 ABM comprises the CDR sequences of CD3-4. In some embodiments, a CD3 ABM comprises the CDR sequences of CD3-5. In some embodiments a CD3 ABM comprises the CDR sequences of CD3-6. In some embodiments, a CD3 ABM comprises the CDR sequences of CD3-7. In some embodiments, a CD3 ABM comprises the CDR sequences of CD3-8. In some embodiments, a CD3 ABM comprises the CDR sequences of CD3-9. In some embodiments, a CD3 ABM comprises the CDR sequences of CD3-10. In some embodiments, a CD3 ABM comprises the CDR sequences of CD3-11. In some embodiments, a CD3 ABM comprises the CDR sequences of CD3-12. In some embodiments, a CD3 ABM comprises the CDR sequences of CD3-13. In some embodiments, a CD3 ABM comprises the CDR sequences of CD3-14. In some embodiments, a CD3 ABM comprises the CDR sequences of CD3-15. In some embodiments, a CD3 ABM comprises the CDR sequences of CD3-16. In some embodiments, a CD3 ABM comprises the CDR sequences of CD3-17. In some embodiments, a CD3 ABM comprises the CDR sequences of CD3-18. In some embodiments, a CD3 ABM comprises the CDR sequences of CD3-19. In some embodiments, a CD3 ABM comprises the CDR sequences of CD3-20. In some embodiments, a CD3 ABM comprises the CDR sequences of CD3-21. In some embodiments, a CD3 ABM comprises the CDR sequences of CD3-22. In some embodiments, a CD3 ABM comprises the CDR sequences of CD3-23. In some embodiments, a CD3 ABM comprises the CDR sequences of CD3-24. In some embodiments, a CD3 ABM comprises the CDR sequences of CD3-25. In some embodiments, a CD3 ABM comprises the CDR sequences of CD3-26. In some embodiments, a CD3 ABM comprises the CDR sequences of CD3-27. In some embodiments, a CD3 ABM comprises the CDR sequences of CD3-28. In some embodiments, a CD3 ABM comprises the CDR sequences of CD3-29. In some embodiments, a CD3 ABM comprises the CDR sequences of CD3-30. In some embodiments, a CD3 ABM comprises the CDR sequences of CD3-31. In some embodiments, a CD3 ABM comprises the CDR sequences of CD3-32. In some embodiments, a CD3 ABM comprises the CDR sequences of CD3-33. In some embodiments, a CD3 ABM comprises the CDR sequences of CD3-34. In some embodiments, a CD3 ABM comprises the CDR sequences of CD3-35. In some embodiments, a CD3 ABM comprises the CDR sequences of CD3-36. In some embodiments, a CD3 ABM comprises the CDR sequences of CD3-37. In some embodiments, a CD3 ABM comprises the CDR sequences of CD3-38. In some embodiments, a CD3 ABM comprises the CDR sequences of CD3-39. In some embodiments, a CD3 ABM comprises the CDR sequences of CD3-40. In some embodiments, a CD3 ABM comprises the CDR sequences of CD3-41. In some embodiments, a CD3 ABM comprises the CDR sequences of CD3-42. In some embodiments, a CD3 ABM comprises the CDR sequences of CD3-43. In some embodiments, a CD3 ABM comprises the CDR sequences of CD3-44. In some embodiments, a CD3 ABM comprises the CDR sequences of CD3-45. In some embodiments, a CD3 ABM comprises the CDR sequences of CD3-46. In some embodiments, a CD3 ABM comprises the CDR sequences of CD3-47. In some embodiments, a CD3 ABM comprises the CDR sequences of CD3-48. In some embodiments, a CD3 ABM comprises the CDR sequences of CD3-49. In some embodiments, a CD3 ABM comprises the CDR sequences of CD3-50. In some embodiments, a CD3 ABM comprises the CDR sequences of CD3-51. In some embodiments, a CD3 ABM comprises the CDR sequences of CD3-52. In some embodiments, a CD3 ABM comprises the CDR sequences of CD3-53. In some embodiments, a CD3 ABM comprises the CDR sequences of CD3-54. In some embodiments, a CD3 ABM comprises the CDR sequences of CD3-55. In some embodiments, a CD3 ABM comprises the CDR sequences of CD3-56. In some embodiments, a CD3 ABM comprises the CDR sequences of CD3-57. In some embodiments, a CD3 ABM comprises the CDR sequences of CD3-58. In some embodiments, a CD3 ABM comprises the CDR sequences of CD3-59. In some embodiments, a CD3 ABM comprises the CDR sequences of CD3-60. In some embodiments, a CD3 ABM comprises the CDR sequences of CD3-61. In some embodiments, a CD3 ABM comprises the CDR sequences of CD3-62. In some embodiments, a CD3 ABM comprises the CDR sequences of CD3-63. In some embodiments, a CD3 ABM comprises the CDR sequences of CD3-64. In some embodiments, a CD3 ABM comprises the CDR sequences of CD3-65. In some embodiments, a CD3 ABM comprises the CDR sequences of CD3-66. In some embodiments, a CD3 ABM comprises the CDR sequences of CD3-67. In some embodiments, a CD3 ABM comprises the CDR sequences of CD3-68. In some embodiments, a CD3 ABM comprises the CDR sequences of CD3-69. In some embodiments, a CD3 ABM comprises the CDR sequences of CD3-70. In some embodiments, a CD3 ABM comprises the CDR sequences of CD3-71. In some embodiments, a CD3 ABM comprises the CDR sequences of CD3-72. In some embodiments, a CD3 ABM comprises the CDR sequences of CD3-73. In some embodiments, a CD3 ABM comprises the CDR sequences of CD3-74. In some embodiments, a CD3 ABM comprises the CDR sequences of CD3-75. In some embodiments, a CD3 ABM comprises the CDR sequences of CD3-76. In some embodiments, a CD3 ABM comprises the CDR sequences of CD3-77. In some embodiments, a CD3 ABM comprises the CDR sequences of CD3-78. In some embodiments, a CD3 ABM comprises the CDR sequences of CD3-79. In some embodiments, a CD3 ABM comprises the CDR sequences of CD3-80. In some embodiments, a CD3 ABM comprises the CDR sequences of CD3-81. In some embodiments, a CD3 ABM comprises the CDR sequences of CD3-82. In some embodiments, a CD3 ABM comprises the CDR sequences of CD3-83. In some embodiments, a CD3 ABM comprises the CDR sequences of CD3-84. In some embodiments, a CD3 ABM comprises the CDR sequences of CD3-85. In some embodiments, a CD3 ABM comprises the CDR sequences of CD3-86. In some embodiments, a CD3 ABM comprises the CDR sequences of CD3-87. In some embodiments, a CD3 ABM comprises the CDR sequences of CD3-88. In some embodiments, a CD3 ABM comprises the CDR sequences of CD3-89. In some embodiments, a CD3 ABM comprises the CDR sequences of CD3-90. In some embodiments, a CD3 ABM comprises the CDR sequences of CD3-91. In some embodiments, a CD3 ABM comprises the CDR sequences of CD3-92. In some embodiments, a CD3 ABM comprises the CDR sequences of CD3-93. In some embodiments, a CD3 ABM comprises the CDR sequences of CD3-94. In some embodiments, a CD3 ABM comprises the CDR sequences of CD3-95. In some embodiments, a CD3 ABM comprises the CDR sequences of CD3-96. In some embodiments, a CD3 ABM comprises the CDR sequences of CD3-97. In some embodiments, a CD3 ABM comprises the CDR sequences of CD3-98. In some embodiments, a CD3 ABM comprises the CDR sequences of CD3-99. In some embodiments, a CD3 ABM comprises the CDR sequences of CD3-100. In some embodiments, a CD3 ABM comprises the CDR sequences of CD3-101. In some embodiments, a CD3 ABM comprises the CDR sequences of CD3-102. In some embodiments, a CD3 ABM comprises the CDR sequences of CD3-103. In some embodiments, a CD3 ABM comprises the CDR sequences of CD3-104. In some embodiments, a CD3 ABM comprises the CDR sequences of CD3-105. In some embodiments, a CD3 ABM comprises the CDR sequences of CD3-106. In some embodiments, a CD3 ABM comprises the CDR sequences of CD3-107. In some embodiments, a CD3 ABM comprises the CDR sequences of CD3-108. In some embodiments, a CD3 ABM comprises the CDR sequences of CD3-109. In some embodiments, a CD3 ABM comprises the CDR sequences of CD3-110. In some embodiments, a CD3 ABM comprises the CDR sequences of CD3-111. In some embodiments, a CD3 ABM comprises the CDR sequences of CD3-112. In some embodiments, a CD3 ABM comprises the CDR sequences of CD3-113. In some embodiments, a CD3 ABM comprises the CDR sequences of CD3-114. In some embodiments, a CD3 ABM comprises the CDR sequences of CD3-115. In some embodiments, a CD3 ABM comprises the CDR sequences of CD3-116. In some embodiments, a CD3 ABM comprises the CDR sequences of CD3-117. In some embodiments, a CD3 ABM comprises the CDR sequences of CD3-118. In some embodiments, a CD3 ABM comprises the CDR sequences of CD3-119. In some embodiments, a CD3 ABM comprises the CDR sequences of CD3-120. In some embodiments, a CD3 ABM comprises the CDR sequences of CD3-121. In some embodiments, a CD3 ABM comprises the CDR sequences of CD3-122. In some embodiments, a CD3 ABM comprises the CDR sequences of CD3-123. In some embodiments, a CD3 ABM comprises the CDR sequences of CD3-124. In some embodiments, a CD3 ABM comprises the CDR sequences of CD3-125. In some embodiments, a CD3 ABM comprises the CDR sequences of CD3-126. In some embodiments, a CD3 ABM comprises the CDR sequences of CD3-127. In some embodiments, a CD3 ABM comprises the CDR sequences of CD3-128.

A TBM of the disclosure can comprise the complete heavy and light variable sequences of any of CD3-1 to CD3-128. In some embodiments, a TBM of the disclosure comprises a CD3 ABM which comprises the VH and VL sequences of CD3-1. In some embodiments, a TBM of the disclosure comprises a CD3 ABM which comprises the VH and VL sequences of CD3-1. In some embodiments, a TBM of the disclosure comprises a CD3 ABM which comprises the VH and VL sequences of CD3-2. In some embodiments, a TBM of the disclosure comprises a CD3 ABM which comprises the VH and VL sequences of CD3-3. In some embodiments, a TBM of the disclosure comprises a CD3 ABM which comprises the VH and VL sequences of CD3-4. In some embodiments, a TBM of the disclosure comprises a CD3 ABM which comprises the VH and VL sequences of CD3-5. In some embodiments, a TBM of the disclosure comprises a CD3 ABM which comprises the VH and VL sequences of CD3-6. In some embodiments, a TBM of the disclosure comprises a CD3 ABM which comprises the VH and VL sequences of CD3-7. In some embodiments, a TBM of the disclosure comprises a CD3 ABM which comprises the VH and VL sequences of CD3-8. In some embodiments, a TBM of the disclosure comprises a CD3 ABM which comprises the VH and VL sequences of CD3-9. In some embodiments, a TBM of the disclosure comprises a CD3 ABM which comprises the VH and VL sequences of CD3-10. In some embodiments, a TBM of the disclosure comprises a CD3 ABM which comprises the VH and VL sequences of CD3-11. In some embodiments, a TBM of the disclosure comprises a CD3 ABM which comprises the VH and VL sequences of CD3-12. In some embodiments, a TBM of the disclosure comprises a CD3 ABM which comprises the VH and VL sequences of CD3-13. In some embodiments, a TBM of the disclosure comprises a CD3 ABM which comprises the VH and VL sequences of CD3-14. In some embodiments, a TBM of the disclosure comprises a CD3 ABM which comprises the VH and VL sequences of CD3-15. In some embodiments, a TBM of the disclosure comprises a CD3 ABM which comprises the VH and VL sequences of CD3-16. In some embodiments, a TBM of the disclosure comprises a CD3 ABM which comprises the VH and VL sequences of CD3-17. In some embodiments, a TBM of the disclosure comprises a CD3 ABM which comprises the VH and VL sequences of CD3-18. In some embodiments, a TBM of the disclosure comprises a CD3 ABM which comprises the VH and VL sequences of CD3-19. In some embodiments, a TBM of the disclosure comprises a CD3 ABM which comprises the VH and VL sequences of CD3-20. In some embodiments, a TBM of the disclosure comprises a CD3 ABM which comprises the VH and VL sequences of CD3-21. In some embodiments, a TBM of the disclosure comprises a CD3 ABM which comprises the VH and VL sequences of CD3-22. In some embodiments, a TBM of the disclosure comprises a CD3 ABM which comprises the VH and VL sequences of CD3-23. In some embodiments, a TBM of the disclosure comprises a CD3 ABM which comprises the VH and VL sequences of CD3-24. In some embodiments, a TBM of the disclosure comprises a CD3 ABM which comprises the VH and VL sequences of CD3-25. In some embodiments, a TBM of the disclosure comprises a CD3 ABM which comprises the VH and VL sequences of CD3-26. In some embodiments, a TBM of the disclosure comprises a CD3 ABM which comprises the VH and VL sequences of CD3-27. In some embodiments, a TBM of the disclosure comprises a CD3 ABM which comprises the VH and VL sequences of CD3-28.

6.5.2. TCR-α/β ABMs

The TBMs of the disclosure can contain an ABM that specifically binds to the TCR-α chain, the TCR-β chain, or the TCR-αβ dimer. Exemplary anti-TCR-α/β antibodies are known in the art (see, e.g., US 2012/0034221; Borst et al., 1990, Hum Immunol. 29(3):175-88 (describing antibody BMA031), the contents of each of which are incorporated herein by reference). The VH, VL, and Kabat CDR sequences of antibody BMA031 as described in US 2012/0034221 are provided in Table 8.

TABLE 8
BMA031 sequences

| | | SEQ |
|---|---|---|
| | | ID |
| Domain | Sequence | NO: |
| BMA031 | KASGYKFTSYVMH | 241 |
| CDR-H1 | | |
| BMA031 | YINPYNDVTKYNEKFK | 242 |
| CDR-H2 | | |
| BMA031 | GSYYDYDGFVY | 243 |
| CDR-H3 | | |
| BMA031 | SATSSVSYMH | 244 |
| CDR-L1 | | |
| BMA031 | DTSKLAS | 150 |
| CDR-L2 | | |
| BMA031 | QQWSSNPLT | 179 |
| CDR-L3 | | |
| BMA031 | EVQLQQSGPELVKPGASVKMSCKASGYKFTSYVM | 245 |
| VH | HWVKQKPGQGLEWIGYINPYNDVTKYNEKFKGKA | |
| | TLTSDKSSSTAYMELSSLTSEDSAVHYCARGSYY | |
| | DYDGFVYWGQGTLVTVSA | |
| BMA031 | QIVLTQSPAIMSASPGEKVTMTCSATSSVSYMHW | 246 |
| VL | YQQKSGTSPKRWIYDTSKLASGVPARFSGSGSGT | |
| | SYSLTISSMEAEDAATYYCQQWSSNPLTFGAGTK | |
| | LELK | |

In an embodiment, a TCR ABM can comprise the CDR sequences of antibody BMA031. In other embodiments, a TCR ABM can comprise the VH and VL sequences of antibody BMA031.

6.5.3. TCR-γ/δ ABMs

The TBMs of the disclosure can contain an ABM that specifically binds to the TCR-γ chain, the TCR-δ chain, or the TCR-γδ dimer. Exemplary anti-TCR-γ/δ antibodies are known in the art (see, e.g., U.S. Pat. No. 5,980,892 (describing OTCS1, produced by the hybridoma deposited with the ATCC as accession number HB 9578), the contents of which are incorporated herein by reference).

### 6.6. CD2 ABMs

6.6.1. Immunoglobulin-Based CD2 ABMs

In some embodiments, a TBM of the disclosure can comprise a ABM which is an anti-CD2 antibody or an antigen-binding domain thereof. Exemplary anti-CD2 antibodies are known in the art (see, e.g., U.S. Pat. No. 6,849,258, CN102827281A, US 2003/0139579 A1, and U.S. Pat. No. 5,795,572). Table 9 provides exemplary CDR, VH, and VL sequences that can be included in anti-CD2 antibodies or antigen-binding fragments thereof, for use in TBMs of the disclosure.

TABLE 9
Immunoglobulin Based CD2 Binders

| | | | SEQ |
|---|---|---|---|
| | Do- | | ID |
| Name | main | Sequence | NO: |
| CD2-1 | CDR- | EYYMY (Rat Lo-CD2a = BTI-322 | 247 |
| | H1 | from FIG. 33 of | |
| | | U.S. Pat. No. 6,849,258) | |
| CD2-1 | CDR- | RIDPEDGSIDYVEKFKK (Rat Lo- | 248 |
| | H2 | CD2a = BTI-322 from FIG. | |
| | | 33 of U.S. Pat. No. 6,849,258) | |
| CD2-1 | CDR- | GKFNYRFAY (Rat Lo-CD2a = | 249 |
| | H3 | BTI-322 from FIG. 33 of | |
| | | U.S. Pat. No. 6,849,258) | |
| CD2-1 | CDR- | RSSQSLLHSSGNTYLN (Rat Lo- | 250 |
| | L1 | CD2a = BTI-322 from FIG. | |
| | | 31 of U.S. Pat. No. 6,849,258) | |
| CD2-1 | CDR- | LVSKLES (Rat Lo-CD2a = | 251 |
| | L2 | BTI-322 from FIG. 31 of | |
| | | U.S. Pat. No. 6,849,258) | |
| CD2-1 | CDR- | QFTHYPYT (Rat Lo-CD2a = | 252 |
| | L3 | BTI-322 from FIG. 31 of | |
| | | U.S. Pat. No. 6,849,258) | |
| CD2-1 | VH | EVQLQQSGPELQRPGASVKLSCKASGY | 253 |
| | | IFTEYYMYWVKQRPKQGLELVGRIDPE | |
| | | DGSIDYVEKFKKKATLTADTSSNTAYM | |
| | | QLSSLTSEDTATYFCARGKFNYRFAYW | |
| | | GQGTLVTVSS (SEQ ID NO: 100 | |
| | | of U.S. Pat. No. 6,849,258) | |
| CD2-1 | VL | DVVLTQTPPTLLATIGQSVSISCRSSQ | 254 |
| | | SLLHSSGNTYLNWLLQRTGQSPQPLIY | |
| | | LVSKLESGVPNRFSGSGSGTDFTLKIS | |
| | | GVEAEDLGVYYCMQFTHYPYTFGAGTK | |
| | | LELK (Rat Lo-CD2a Vk from | |
| | | SEQ ID NO: 92, without signal | |
| | | sequence as shown in FIG. 31 | |
| | | of U.S. Pat. No. 6,849,258) | |
| hu1CD2- | VH | QVQLVQSGAEVKKPGASVKVSCKASGY | 255 |
| 1 | | TFTEYYMYWVRQAPGQGLELMGRIDPE | |
| | | DGSIDYVEKFKKKVTLTADTSSSTAYM | |
| | | ELSSLTSDDTAVYYCARGKFNYRFAYW | |
| | | GQGTLVTVSS (SEQ ID NO: 101 | |
| | | of U.S. Pat. No. 6,849,258) | |
| | VL | DVVMTQSPPSLLVTLGQPASISCRSSQ | 256 |
| | | SLLHSSGNTYLNWLLQRPGQSPQPLIY | |
| | | LVSKLESGVPDRFSGSGSGTDFTLKIS | |
| | | GVEAEDVGVYYCMQFTHYPYTFGQGTK | |
| | | LEIK (SEQ ID NO: 96 of | |
| | | U.S.Pat. No. 6,849,258) | |
| hu2CD2- | VH | EVQLQQSGPELQRPGASVKLSCKASGY | 257 |
| 1 | | IFTEYYMYWVKQRPKQGLELVGRIDPE | |
| | | DGSIDYVEKFKKKATLTADTSSNTAYM | |
| | | QLSSLTSEDTATYFCARGKFNYRFAYW | |
| | | GQGTLVTVSS (Vh of MEDI-507; | |
| | | SEQ ID NO: 105 of U.S. Pat. | |
| | | No. 6,849,258) | |
| | VL | DVVMTQSPPSLLVTLGQPASISCRSSQ | 256 |
| | | SLLHSSGNTYLNWLLQRPGQSPQPLIY | |
| | | LVSKLESGVPDRFSGSGSGTDFTLKIS | |
| | | GVEAEDVGVYYCMQFTHYPYTFGQGTK | |
| | | LEIK (SEQ ID NO: 96 of U.S. | |
| | | Pat. No. 6,849,258)(same as | |
| | | hu1CD2-1) | |

In some embodiments, a CD2 ABM comprises the CDR sequences of CD2-1 (SEQ ID NOS: 247-252). In some embodiments, a CD2 ABM comprises the heavy and light chain variable sequences of CD2-1 (SEQ ID NOS: 253-254). In some embodiments, a CD2 ABM comprises the heavy and light chain variable sequences of hu1CD2-1 (SEQ ID NOS: 255-256). In some embodiments, a CD2 ABM comprises the heavy and light chain variable sequences of hu2CD2-1 (SEQ ID NOS: 257 and 256, respectively).

In other embodiments, a CD2 ABM can comprise the CDR sequences of antibody 9D1 produced by the hybridoma deposited with the Chinese Culture Collection Committee General Microbiology Center on May 16, 2012 with accession no. CGMCC 6132, and which is described in CN102827281A. In other embodiments, a CD2 ABM can comprise the CDR sequences of antibody LO-CD2b produced by the hybridoma deposited with the American Type Culture Collection on Jun. 22, 1999 with accession no. PTA-802, and which is described in US 2003/0139579 A1. In yet other embodiments, a CD2 ABM can comprise the CDR sequences of the CD2 SFv-Ig produced by expression of the construct cloned in the recombinant E. coli deposited with the ATCC on Apr. 9, 1993 with accession no. 69277, and which is described in U.S. Pat. No. 5,795,572.

In other embodiments, a CD2 ABM can comprise the VH and VL sequences of antibody 9D1. In other embodiments, a CD2 ABM can comprise the VH and VL sequences of antibody LO-CD2b. In yet other embodiments, a CD2 ABM can comprise the VH and VL sequences of the CD2 SFv-Ig produced by expression of the construct cloned in the recombinant E. coli having ATCC accession no. 69277.

6.6.2. CD58-Based CD2 ABMs

In certain aspects the present disclosure provides a TBM comprising a CD2 ABM which is a ligand. The CD2 ABM specifically binds to human CD2, whose natural ligand is CD58, also known as LFA-3. CD58/LFA-3 proteins are glycoproteins that are expressed on the surfaces of a variety of cell types (Dustin et al., 1991, Annu. Rev. Immunol. 9:27) and play roles in mediating T-cell interactions with APCs in both antigen-dependent and antigen-independent manners (Wallner et al., 1987, J. Exp. Med. 166:923). Accordingly, in certain aspects, the CD2 ABM is a CD58 moiety. As used herein, a CD58 moiety comprises an amino acid sequence comprising at least 70% sequence identity to a CD2-binding portion of CD58, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a CD2-binding portion of CD58. The sequence of human CD58 has the Uniprot identifier P19256 (www.uniprot.org/uniprot/P19256). It has been established that CD58 fragments containing amino acid residues 30-123 of full length CD58 (i.e., the sequence designated as CD58-4 in Table 10 below) are sufficient for binding to CD2. Wang et al., 1999, Cell 97:791-803. Accordingly, in certain aspects, a CD58 moiety comprises an amino acid sequence comprising at least 70% sequence identity to amino acids 30-123 of CD58, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence designated CD58-4.

The interactions between CD58 and CD2 have been mapped through x-ray crystallography and molecular modeling. The substitution of residues E25, K29, K30, K32, D33, K34, E37, D84 and K87 (with numbering referring to the in the mature polypeptide) reduces binding to CD2. Ikemizu et al., 1999, Proc. Natl. Acad. Sci. USA 96:4289-94. Accordingly, in preferred embodiments the CD58 moiety of the disclosure retains the wild type residues at E25, K29, K30, K32, D33, K34, E37, D84 and K87.

In contrast, the following substitutions (with numbering referring to the full length polypeptide) did not impact binding to CD2: F29S; V37K; V49Q; V86K; T113S; and L121G. Accordingly, a CD58 moiety of the disclosure can include one, two, three, four, five or all six of the foregoing substitutions.

Exemplary CD58 moieties are provided in Table 10 below:

TABLE 10
CD58 sequences

| | | | SEQ |
|---|---|---|---|
| | Descrip- | | ID |
| Name | tion | Sequence | NO: |
| CD58- | Full | MVAGSDAGRA LGVLSVVCLL | 258 |
| 1 | length | HCFGFISCFS QQIYGVVYGN | |
| | CD58, | VTFHVPSNVP LKEVLWKKQK | |
| | including | DKVAELENSE FRAFSSFKNR | |
| | signal | VYLDTVSGSL TIYNLTSSDE | |
| | sequence | DEYEMESPNI TDTMKFFLYV | |
| | | LESLPSPTLT CALTNGSIEV | |
| | | QCMIPEHYNS HRGLIMYSWD | |
| | | CPMEQCKRNS TSIYFKMEND | |
| | | LPQKIQCTLS NPLFNTTSSI | |
| | | ILTTCIPSSG HSRHRYALIP | |
| | | IPLAVITTCI VLYMNGILKC | |
| | | DRKPDRTNSN | |
| CD58- | Extra- | FS QQIYGVVYGN VTFHVPSNVP | 259 |
| 2 | cellular | LKEVLWKKQK DKVAELENSE | |
| | domain of | FRAFSSFKNR VYLDTVSGSL | |
| | CD58, | TIYNLTSSDE DEYEMESPNI | |
| | corres- | TDTMKFFLYV LESLPSPTLT | |
| | ponding | CALTNGSIEV QCMIPEHYNS | |
| | to amino | HRGLIMYSWD CPMEQCKRNS | |
| | acids 29- | TSIYFKMEND LPQKIQCTLS | |
| | 215 of | NPLFNTTSSI ILTTCIPSSG | |
| | CD58 (WT) | HSRHR | |
| CD58- | Extra- | BS QQIYGVJYGN VTFHVPSNOP | 260 |
| 3 | cellular | LKEVLWKKQK DKVAELENSE | |
| | domain of | FRAFSSFKNR VYLDTUSGSL | |
| | CD58, | TIYNLTSSDE DEYEMESPNI | |
| | corres- | TDXMKFFLYV ZESLPSPTLT | |
| | ponding | CALTNGSIEV QCMIPEHYNS | |
| | to amino | HRGLIMYSWD CPMEQCKRNS | |
| | acids 29- | TSIYFKMEND LPQKIQCTLS | |
| | 215 of | NPLFNTTSSI ILTTCIPSSG | |
| | CD58 | HSRHR | |
| | (with | B = F or S | |
| | permitted | J = V or K | |
| | substi- | O = V or Q | |
| | tutions) | U = V or K | |
| | | X = T or S | |
| | | Z = L or G | |
| CD58- | Amino | S QQIYGVVYGN VTFHVPSNVP | 261 |
| 4 | acids 30- | LKEVLWKKQK DKVAELENSE | |
| | 123 (WT) | FRA | |
| CD58- | Amino | S QQIYGVJYGN VTFHVPSNOP | 262 |
| 5 | acids 30- | LKEVLWKKQK DKVAELENSE | |
| | 123 (with | FRA | |
| | permitted | J = V or K | |
| | substi- | O = V or Q | |
| | tutions) | | |

6.6.3. CD48-Based CD2 ABMs

In certain aspects the present disclosure provides a TBM comprising a CD2 ABM which is CD48 moiety. As used herein, a CD48 moiety comprises an amino acid sequence comprising at least 70% sequence identity to a CD2-binding portion of CD48, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a CD2-binding portion of CD48. The sequence of human CD48 has the Uniprot identifier P09326 (www.uniprot.org/uniprot/P09326), which includes a signal peptide (amino acids 1-26) and a GPI anchor (amino acids 221-243). In certain aspects, a CD48 moiety comprises an amino acid sequence comprising at least 70% sequence identity (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to the amino acid sequence of consisting of amino acids 27-220 of Uniprot identifier P09326. Human CD48 has an Ig-like C2-type I domain (amino acids 29-127 of Uniprot identifier P09326) and a Ig-like C2 type 2 domain (amino acids 132-212 of Uniprot identifier P09326). Accordingly, in some embodiments, a CD48 moiety comprises an amino acid sequence comprising at least 70% sequence identity (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to the amino acid sequence of consisting of amino acids 29-212 of Uniprot identifier P09326, to the C2-type I domain (amino acids 29-127 of Uniprot identifier P09326) and/or to the Ig-like C2 type 2 domain (amino acids 132-212 of Uniprot identifier P09326). A CD48 moiety can in some embodiments comprise one or more natural variants relative to the sequence of Uniprot identifier P09326. For example, a CD48 moiety can include a E102Q substitution. As another example, a CD48 moiety can comprise an amino acid sequence corresponding to a CD-48 isoform or a CD2 binding portion thereof, e.g., the isoform having Uniprot identifier P09326-2 or a CD2 binding portion thereof.

### 6.7. Tumor-Associated Antigen ABMs

The TBMs of the disclosure comprise at least one ABMs that bind specifically to a tumor-associated antigen (TAA). Preferably, the TAA is a human TAA. The antigen may or may not be present on normal cells. In certain embodiments, the TAA is preferentially expressed or upregulated on tumor cells as compared to normal cells. In other embodiments, the TAA is a lineage marker.

It is anticipated that any type of tumor and any type of TAA may be targeted by the TBMs of the disclosure. Exemplary types of cancers that may be targeted include acute lymphoblastic leukemia, acute myelogenous leukemia, biliary cancer, B-cell leukemia, B-cell lymphoma, biliary cancer, bone cancer, brain cancer, breast cancer, triple-negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and other urinary bladder cancers. However, the skilled artisan will realize that TAAs are known for virtually any type of cancer.

Exemplary TAAs for which a TBM of the disclosure can be created include ABCF1; ACVR1; ACVR1B; ACVR2; ACVR2B; ACVRL1; ADORA2A; ADRB3; Aggrecan; AGR2; AICDA; AIF1; AIG1; AKAP1; AKAP2; ALK; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; Cadherin 17; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD32b; CD40; CD40L; CD44; CD45RB; CD52; CD69; CD72; CD74; CD79A; CD79B; CD8; CD80; CD81; CD83; CD86; CD97; CD179a; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN6; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10(IP-10); CXCL11 (1-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; CGRP; C1q; C1r; C1; C4a; C4b; C2a; C2b; C3a; C3b; DAB21P; DES; DKFZp451J0118; DNCL1; DPP4; E-selectin; E2F1; ECGF1; EDG1; EFNA1; EFNA3; EFNB2; EGF; EGFR; EGFRvlll; ELAC2; ENG; ENO1; ENO2; ENO3; EPHB4; EPO; ERBB2 (Her-2); EREG; ERK8; ESR1; ESR2; F3 (TF); Factor VII; Factor IX; Factor V; Factor Vila; Factor Factor X; Factor XII; Factor XIII; FADD; FasL; FASN; FCER1A; FCER2; Fc gamma receptor; FCGR3A; FCRL5; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; Folate receptor alpha; Folate receptor beta; FOS; FOSL1 (FRA-1); Fucosyl GM1; FY (DARC); GABRP (GABAa); GAGEB1; GAGEC1; GALNAC4S-65T; GATA3; GDF5; GFI1; GGT1; GM-CSF; GloboH; GNAS1; GNRH1; GPNMB; GPR2 (CCR10); GPR20; GPR31; GPR44; GPR64; GPR81 (FKSG80); GPRC5D; GRCC10 (010); GRP; GSN (Gelsolin); GSTP1; glycoprotein (gP) IIb/IIIa; HAVCR1; HAVCR2; HDAC4; HDAC5; HDAC7A; HDAC9; Her2; HER3; HGF; HIF1A; HIP1; histamine and histamine receptors; HLA-A; HLA-DRA; HM74; HMGB1; HMOX1; HMWMAA; HUMCYT2A; ICEBERG; ICOSL; ID2; IFN-α; IFNA1; IFNA2; IFNA4; IFNA5; IFNA6; IFNA7; IFNB1; IFN-γ; IFNW1; IGBP1; IGF1; IGF1R; IGF2; IGFBP2; IGFBP3; IGFBP6; IL-1; IL-α; IL-1-β; IL10; IL10 RA; IL10 RB; IL11; IL11 RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1 HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); L-selectin; LAMAS; LEP (leptin); Lingo-p75; Lingo-Troy; LRP6; LPS; LTA (TNF-b); LTB; LTB4R (GPR16); LTB4R2; LTBR; LY6K; LYPD8; MACMARCKS; MAG or Omgp; MAP2K7 (c-Jun); MDK; mesothelin; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NKG2D; NFKB1; NFKB2; NGF; NGFB (NGF); NGFR; NgR-Lingo; NgR-Nogo66 (Nogo); NgR-p75; NgR-Troy; NME1 (NM23A); NOX5; NPPB; NROB1; NROB2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; NY-BR-1; o-acetyl-GD2; ODZ1; OPRD1; OR51E2; P2RX7; PANX3; PAP; PART1; PATE; PAWR; PCA3; PCNA; PDGFA; PDGFB; PECAM1; PF4 (CXCL4); PGE2; PGF; PGR; phosphacan; PIAS2; PIK3CG; PLAC1; plasminogen activator; PLAU (uPA); PLG; PLXDC1; polysialic acid; PPBP (CXCL7); PPID; PR1; PRKCQ; PRKD1; PRL; PROC; Protein C; PROK2; PSAP; PSCA; PTAFR; PTEN; PTGS2 (COX-2); PTN; RAC2 (p21Rac2); RAGE; RARB; RGS1; RGS13; RGS3; RNF110 (ZNF144); ROBO2; SUI0A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINBS (maspin); SERPINE1 (PAI-1); SERPINF1; SHBG; SLA2; SLC2A2; SLC33A1; SLC34A2; SLC39A6; SLC43A1; SLIT2; SLITRK6; SPP1; SPRR1B (Spr1); ST6GAL1; STAB1; STATE; STEAP; STEAP2; substance P; TACSTD2; TB4R2; TBX21; TCP10; TDGF1; TEK; TEM1/CD248; TEM7R; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-α; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase ha); TP53; TPM1; TPM2; TRADD; TRAF1; TRAF2; TRAF3; TRAF4; TRAF5; TRAF6; TREM1; TREM2; TRPC6; TSHR; TSLP; TWEAK; thrombomodulin; thrombin; UPK2; VEGF; VEGFB; VEGFC; versican; VHL C5; VLA-4; XCL1 (lymphotactin); XCL2 (SCM-1b); XCR1 (GPRS/CCXCR1); YY1; and ZFPM2.

In some embodiments, the TAA is ADRB3. In some embodiments, the TAA is AKAP-4. In some embodiments, the TAA is ALK. In some embodiments, the TAA is androgen receptor. In some embodiments, the TAA is B7H3. In some embodiments, the TAA is BCMA. In some embodiments, the TAA is BORIS. In some embodiments, the TAA is BST2. In some embodiments, the TAA is Cadherin17. In some embodiments, the TAA is CAIX. In some embodiments, the TAA is CD171. In some embodiments, the TAA is CD179a. In some embodiments, the TAA is CD19. In some embodiments, the TAA is CD20. In some embodiments, the TAA is CD22. In some embodiments, the TAA is CD24. In some embodiments, the TAA is CD30. In some embodiments, the TAA is CD300LF. In some embodiments, the TAA is CD32b. In some embodiments, the TAA is CD33. In some embodiments, the TAA is CD38. In some embodiments, the TAA is CD44v6. In some embodiments, the TAA is CD72. In some embodiments, the TAA is CD79a. In some embodiments, the TAA is CD79b. In some embodiments, the TAA is CD97. In some embodiments, the TAA is CEA. In some embodiments, the TAA is CLDN6. In some embodiments, the TAA is CLEC12A. In some embodiments, the TAA is CLL-1. In some embodiments, the TAA is CS-1. In some embodiments, the TAA is CXORF61. In some embodiments, the TAA is Cyclin B1. In some embodiments, the TAA is CYP1B1. In some embodiments, the TAA is EGFR. In some embodiments, the TAA is EGFRvIII. In some embodiments, the TAA is EMR2. In some embodiments, the TAA is EPCAM. In some embodiments, the TAA is EphA2. In some embodiments, the TAA is EphB2. In some embodiments, the TAA is ERBB2. In some embodiments, the TAA is ERG (TMPRSS2 ETS fusion gene). In some embodiments, the TAA is ETV6-AML. In some embodiments, the TAA is FAP. In some embodiments, the TAA is FCAR. In some embodiments, the TAA is FCRL5. In some embodiments, the TAA is FLT3. In some embodiments, the TAA is FLT3. In some embodiments, the TAA is folate receptor alpha. In some embodiments, the TAA is folate receptor beta. In some embodiments, the TAA is Fos-related antigen 1. In some embodiments, the TAA is fucosyl GM1. In some embodiments, the TAA is GD2. In some embodiments, the TAA is GD2. In some embodiments, the TAA is GD3. In some embodiments, the TAA is GloboH. In some embodiments, the TAA is GM3. In some embodiments, the TAA is gp100Tn. In some embodiments, the TAA is GPC3. In some embodiments, the TAA is GPNMB. In some embodiments, the TAA is GPR20. In some embodiments, the TAA is GPRC5D. In some embodiments, the TAA is GPR64. In some embodiments, the TAA is HAVCR1. In some embodiments, the TAA is HER3. In some embodiments, the TAA is HMWMAA. In some embodiments, the TAA is hTERT. In some embodiments, the TAA is Igf-I receptor. In some embodiments, the TAA is IGLL1. In some embodiments, the TAA is IL-11Ra. In some embodiments, the TAA is IL-13Ra2. In some embodiments, the TAA is KIT. In some embodiments, the TAA is LAIR1. In some embodiments, the TAA is LCK. In some embodiments, the TAA is LewisY. In some embodiments, the TAA is LILRA2. In some embodiments, the TAA is LMP2. In some embodiments, the TAA is LRP6. In some embodiments, the TAA is LY6K. In some embodiments, the TAA is LY75. In some embodiments, the TAA is LYPD8. In some embodiments, the TAA is MAD-CT-1. In some embodiments, the TAA is MAD-CT-2. In some embodiments, the TAA is mesothelin. In some embodiments, the TAA is ML-IAP. In some embodiments, the TAA is MUC1. In some embodiments, the TAA is MYCN. In some embodiments, the TAA is NA17. In some embodiments, the TAA is NCAM. In some embodiments, the TAA is NKG2D. In some embodiments, the TAA is NY-BR-1. In some embodiments, the TAA is o-acetyl-GD2. In some embodiments, the TAA is OR51E2. In some embodiments, the TAA is OY-TES1. In some embodiments, the TAA is a p53 mutant. In some embodiments, the TAA is PANX3. In some embodiments, the TAA is PAX3. In some embodiments, the TAA is PAX5. In some embodiments, the TAA is PDGFR-beta. In some embodiments, the TAA is PLAC1. In some embodiments, the TAA is polysialic acid. In some embodiments, the TAA is PRSS21. In some embodiments, the TAA is PSCA. In some embodiments, the TAA is RhoC. In some embodiments, the TAA is ROR1. In some embodiments, the TAA is a sarcoma translocation breakpoint protein. In some embodiments, the TAA is SART3. In some embodiments, the TAA is SLC34A2. In some embodiments, the TAA is SLC39A6. In some embodiments, the TAA is sLe. In some embodiments, the TAA is SLITRK6. In some embodiments, the TAA is sperm protein 17. In some embodiments, the TAA is SSEA-4. In some embodiments, the TAA is SSX2. In some embodiments, the TAA is TAAG72. In some embodiments, the TAA is TAARP. In some embodiments, the TAA is TACSTD2. In some embodiments, the TAA is TEM1/CD248. In some embodiments, the TAA is TEM7R. In some embodiments, the TAA is TGS5. In some embodiments, the TAA is Tie 2. In some embodiments, the TAA is Tn Ag. In some embodiments, the TAA is TSHR. In some embodiments, the TAA is tyrosinase. In some embodiments, the TAA is UPK2. In some embodiments, the TAA is VEGFR2. In some embodiments, the TAA is WT1. In some embodiments, the TAA is XAGE1.

A TAA ABM can comprise, for example, a ligand- or an antibody-based moiety. For example, in the case of BCMA as a TAA, the ABM can be APRIL, the BCMA ligand, or a portion thereof that binds BCMA, or an anti-BCMA antibody or an antigen-binding fragment thereof. Ligands and antibodies that bind to TAAs are well-known in the art. In the case of antibody-based moieties, the anti-TAA antibody or antigen-binding fragment can comprise, for example, the CDR sequences of an antibody set forth in Table 11. In some embodiments, the anti-TAA antibody or antigen-binding domain thereof has the heavy and light chain variable region sequences of an antibody set forth in Table 11.

TABLE 11
Exemplary Anti-Tumor-Associated Antigen Antibodies

| Target | Examples of Antibody Name and/or Reference(s) and/or Source |
|---|---|
| ALK | antibodies described in e.g., Mino-Kenudson et al., 2010, Clin Cancer Res |
| | 16(5): 1561-1571 |
| B7H3 | MGA271 (Macrogenics) |
| BCMA | Any BCMA antibody described in WO2012163805, WO200112812, or |
| | WO2003062401. |
| CAIX | Antibody clone 303123 (R&D Systems) |
| CD123 | US8,852,551; EP2426148; WO2014138819; WO2016028896; WO2014130635 |
| CD171 | Hong et al., 2014, J Immunother 37(2): 93-104. |
| CD19 | WO2014031687; WO2012079000; WO2014153270; U.S. Pat. No. 7,741,465; the |
| | CD19 binder of Yescarta or Blinatumomab |
| CD20 | Rituximab, Ofatumumab, Ocrelizumab, Veltuzumab, or GA101 |
| CD22 | Haso et al., 2013, Blood, 121(7): 1165-1174; Wayne et al., 2010, Clin Cancer Res |
| | 16(6): 1894-1903; Kato et al., 2013, Leuk Res 37(1): 83-88; Creative BioMart |
| | (creativebiomart.net): MOM-18047-S(P). |
| CD24 | Maliar et al., Gastroenterology 143(5): 1375-1384 (2012) |
| CD30 | Any CD30 antibody described in US7090843 B1, or EP0805871 |
| CD33 | Bross et al., 2001, Clin Cancer Res 7(6): 1490-1496 (Gemtuzumab Ozogamicin, |
| | hP67.6), Caron et al., 1992, Cancer Res 52(24): 6761-6767 (Lintuzumab, HuM195), |
| | Lapusan et al., 2012, Invest New Drugs 30(3): 1121-1131 (AVE9633), Aigner et al., |
| | 2013, Leukemia 27(5): 1107-1115 (AMG330, CD33 BiTE), Dutour et al., 2012, Adv |
| | Hematol 2012: 683065, or Pizzitola et al., 2014, Leukemia |
| | doi: 10.1038/Lue.2014.62. |
| CD38 | Daratumumab (see, e.g., Groen et al., 2010, Blood 116(21): 1261-1262; MOR202 |
| | (see, e.g., U.S. Pat. No. 8,263,746); or any CD38 antibody described in U.S. |
| | Pat. No. 8,362,211. |
| CD44v6 | Casucci et al., 2013, Blood 122(20): 3461-3472. |
| CD97 | antibodies described in, e.g., U.S. Pat. No. 6,846,911; de Groot et al., 2009, |
| | J Immunol 183(6): 4127-4134; antibody from R&D: MAB373 |
| CEA | Chmielewski et al., 2012, Gastoenterology 143(4): 1095-1107. |
| CLDN6 | WO2015069794; IMAB027, mAb, Ganymed Pharmaceuticals |
| CLL-1 | PE-CLL1-hu Cat #353604 (BioLegend); and PE-CLL1 (CLEC12A) Cat #562566 |
| | (BD); WO 2014/051433 A1; U.S. 2016/0368994 A1; U.S. 2013/0295118 A1; |
| | U.S. Pat. No. 8,536,310 B2; Lu et al., 2014, Angewandte Chemie International |
| | Edition 53(37): 9841-9845; Leong et al., 2017, Blood 129(5): 609-618 |
| CS1 | Elotuzumab (BMS), see e.g., Tai et al., 2008, Blood 112(4): 1329-37; Tai et al., |
| | 2007, Blood. 110(5): 1656-63. |
| EGFR | Cetuximab, panitumumab, zalutumumab, nimotuzumab, or matuzumab |
| EGFRvIII | WO2012138475; WO2014130657 |
| EPCAM | MT110, EpCAM-CD3 bispecific Ab (see, e.g., clinicaltrials.gov/ct2/show/ |
| | NCT00635596); Edrecolomab; 3622W94; ING-1; or adecatumumab (MT201). |
| EphA2 | Yu et al., 2014, Mol Ther 22(1): 102-111. |
| Ephrin B2 | Abengozar et al., 2012, Blood 119(19): 4565-4576. |
| ERBB2 | Trastuzumab or pertuzumab. |
| (Her2/neu) | |
| FAP | Ostermann et al., 2008, Clinical Cancer Research 14: 4584-4592 (FAP5), U.S. Pat. |
| | Publication No. 2009/0304718; sibrotuzumab (see e.g., Hofheinz et al., 2003, |
| | Oncology Research and Treatment 26(1): 44-48); and Tran et al., 2013, J Exp Med |
| | 210(6): 1125-1135. |
| FLT3 | Any FLT3 antibody described in WO2011076922, U.S. Pat. No. 5777084, |
| | EP0754230, or US20090297529. |
| Folate | IMGN853, or any folate receptor alpha antibody described in US20120009181; U.S. |
| receptor | Pat. No. 4851332, LK26: U.S. Pat No. 5952484. |
| alpha | |
| Folate | antibodies described in, e.g., US20100297138 ; WO2007/067992 |
| receptor | |
| beta | |
| GD2 | Mujoo et al., Cancer Res. 47(4): 1098-1104 (1987); Cheung et al., Cancer Res |
| | 45(6): 2642-2649 (1985), Cheung et al., J Clin Oncol 5(9): 1430-1440 (1987), |
| | Cheung et al., J Clin Oncol 16(9): 3053-3060 (1998), Handgretinger et al., Cancer |
| | Immunol Immunother 35(3): 199-204 (1992); |
| | mAb 14.18, 14G2a, ch14.18, hu14.18, 3F8, hu3F8, 3G6, 8B6, 60C3, 10B8, |
| | ME36.1, or 8H9 (see e.g., WO2012033885, WO2013040371, WO2013192294, |
| | WO2013061273, WO2013123061, WO2013074916, and WO201385552). |
| | Any GD2 antibody described in U.S. Publication No.: 20100150910 or PCT |
| | Publication No.: WO2011160119. |
| GD3 | Any GD3 antibody described in U.S. Pat. No. 7253263; U.S. Pat. No. 8,207,308; |
| | U.S. 20120276046; EP1013761; WO2005035577; or U.S. Pat. No. 6437098. |
| GloboH | VK9; Kudryashov et al., 1998, Glycoconj J. 15(3): 243-9; Lou et al., 2014, Proc Natl |
| | Acad Sci U.S.A 111(7): 2482-2487; MBr1: Bremer et al., 1984, J Biol Chem |
| | 259: 14773-14777. |
| gp100 | HMB45, NKIbetaB, or any anti-gp100 antibody described in WO2013165940, or |
| | US20130295007 |
| GPRC5D | R&Dsystems: FAB6300A; Lifespan Biosciences: LS-A4180 |
| HMWMAA | antibodies described in, e.g., Kmiecik et al., 2014, Oncoimmunology 3(1): e27185 |
| | (PMID: 24575382) (mAb9.2.27); U.S. Pat. No. 6528481; WO2010033866; U.S. |
| | 20140004124 |
| IGF-I | Any IGF-I receptor antibody described in US8344112 B2; EP2322550 A1; WO |
| receptor | 2006/138315, or PCT/U52006/022995. |
| IL-11Ra | Abcam (cat #ab55262) or Novus Biologicals (cat #EPR5446) |
| IL-13Ra2 | Any IL-13Ra2 antibody described in WO2008/146911, WO2004087758, or |
| | WO2004087758 |
| KIT | Any KIT antibody described in US7915391, US20120288506 |
| KLRG2 | ab121563 (Abcam); B-12 or sc-514346 (Santa Cruz); HPA018199 (Sigma Aldrich) |
| LewisY | Kelly et al., Cancer Biother Radiopharm 23(4): 411-423 (2008) (hu3S193 Ab |
| | (scFvs)); Dolezal et al., Protein Engineering 16(1): 47-56 (2003) (NC10 scFv) |
| LMP2 | Any LMP2 antibody described in U.S. 7,410,640 or U.S. 2005/0129701 |
| LRP6 | WO2009064944, WO2009056634, WO2011119661, WO2011138392, |
| | WO2011138391, WO2013067355, WO2014029752, WO2017093478 |
| Mesothelin | Any mesothelin antibody described in U.S. 20110262448, U.S. 2012/0107933 or |
| | U.S. 9,719,996 |
| MUC1 | SAR566658 |
| NCAM | 2-2B: MAB5324 (EMD Millipore) |
| NY-BR-1 | antibodies described in, e.g., Jager et al., 2007, Appl lmmunohitochem Mol |
| | Morphol 15(1): 77-83 |
| o-acetyl- | 8B6 |
| GD2 | |
| PDGFR- | Abcam ab32570 |
| beta | |
| PLAC1 | antibodies described in, e.g., Ghods et al., 2013, Biotechnol Appl Biochem |
| | doi: 10.1002/bab.1177 |
| Polysialic | antibodies described in e.g., Nagae et al., 2013, J Biol Chem 288(47): 33784- |
| acid | 33796 |
| PRSS21 | Any PRSS21 antibody described in U.S. Pat. No.: 8,080,650. |
| PSCA | Morgenroth et al., Prostate 67(10): 1121-1131 (2007) (scFv 7F5); Nejatollahi et al., |
| | J of Oncology 2013(2013), article ID 839831 (scFv C5-II); or any PSCA antibody |
| | described in U.S. Pat Publication No. 20090311181. |
| PSMA | Parker et al., Protein Expr Purif 89(2): 136-145 (2013), U.S. 20110268656 (J591 |
| | ScFv); Frigerio et al, European J Cancer 49(9): 2223-2232 (2013) (scFvD2B); WO |
| | 2006125481 (mAbs 3/A12, 3/E7 and 3/F11) or single chain antibody fragments |
| | (scFv A5 and D7). |
| ROR1 | Hudecek et al., Olin Cancer Res 19(12): 3153-3164 (2013); or any ROR1 antibody |
| | described in WO 2011159847 or US20130101607. |
| SSEA-4 | MC813 (Cell Signaling) |
| TAG72 | Hombach et al., Gastroenterology 113(4): 1163-1170 (1997) or Abcam ab691. |
| TEM1/ | antibodies described in, e.g., Marty et al., 2006, Cancer Lett 235(2): 298-308; Zhao |
| CD248 | et al., 2011, J Immunol Methods 363(2): 221-232 |
| Tn | Brooks et al., PNAS 107(22): 10056-10061 (2010); Stone et al., Oncolmmunology |
| | 1(6): 863-873(2012); any Tn antibody described in US8,440,798 |
| TSHR | antibodies described in, e.g., Marty et al., 2006, Cancer Lett 235(2): 298-308; Zhao |
| | et al., 2011, J Immunol Methods 363(2): 221-232 |
| Tyrosinase | Any tyrosinase antibody described in U.S. Pat. No. 5843674 or US19950504048. |
| VEGFR2 | Chinnasamy et al., J Clin Invest 120(11): 3953-3968 (2010). |

6.7.1. BCMA

In certain aspects, the present disclosure provides a TBM in which ABM3 BCMA is a tumor necrosis family receptor (TNFR) member expressed on cells of the B-cell lineage. BCMA expression is the highest on terminally differentiated B cells that assume the long lived plasma cell fate, including plasma cells, plasmablasts and a subpopulation of activated B cells and memory B cells. BCMA is involved in mediating the survival of plasma cells for maintaining long-term humoral immunity. The expression of BCMA has been recently linked to a number of cancers, autoimmune disorders, and infectious diseases. Cancers with increased expression of BCMA include some hematological cancers, such as multiple myeloma, Hodgkin's and non-Hodgkin's lymphoma, various leukemias, and glioblastoma.

TBMs comprising a ABM that binds to BCMA can comprise, for example, an anti-BCMA antibody or an antigen-binding domain thereof. The anti-BCMA antibody or antigen-binding domain thereof can comprise, for example, CDR, VH, VL, or scFV sequences set forth in Tables 12A-12G.

TABLE 12A
BCMA Binders - Variable domain and
scFv sequences

| | | | SEQ |
|---|---|---|---|
| Anti- | Do- | | ID |
| body | main | Sequence | NO. |
| BCMA- | VH | EVQLVESGGGLVQPGGSLRLSCAVSGFALSNHGMS | 263 |
| 1 | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSS | |
| | VL | DIQLTQSPSSLSASVGDRVTITCRASQSISSYLNW | 264 |
| | | YQQKPGKAPKLLIYAASSLQSGVPSRFSGSGSGTD | |
| | | FTLTISSLQPEDFATYYCQQSYSTPYTFGQGTKVE | |
| | | IK | |
| | scFv | EVQLVESGGGLVQPGGSLRLSCAVSGFALSNHGMS | 265 |
| | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSSASGGGGSGGRASGGGGSDIQLTQSP | |
| | | SSLSASVGDRVTITCRASQSISSYLNWYQQKPGKA | |
| | | PKLLIYAASSLQSGVPSRFSGSGSGTDFTLTISSL | |
| | | QPEDFATYYCQQSYSTPYTFGQGTKVEIK | |
| BCMA- | VH | QVQLVESGGGLVQPGRSLRLSCAASGFTFSNYAMS | 266 |
| 2 | | WVRQAPGKGLGWVSGISRSGENTYYADSVKGRFTI | |
| | | SRDNSKNTLYLQMNSLRDEDTAVYYCARSPAHYYG | |
| | | GMDVWGQGTTVTVSS | |
| | VL | DIVLTQSPGTLSLSPGERATLSCRASQSISSSFLA | 267 |
| | | WYQQKPGQAPRLLIYGASRRATGIPDRFSGSGSGT | |
| | | DFTLTISRLEPEDSAVYYCQQYHSSPSWTFGQGTK | |
| | | LEIK | |
| | scFv | QVQLVESGGGLVQPGRSLRLSCAASGFTFSNYAMS | 268 |
| | | WVRQAPGKGLGWVSGISRSGENTYYADSVKGRFTI | |
| | | SRDNSKNTLYLQMNSLRDEDTAVYYCARSPAHYYG | |
| | | GMDVWGQGTTVTVSSASGGGGSGGRASGGGGSDIV | |
| | | LTQSPGTLSLSPGERATLSCRASQSISSSFLAWYQ | |
| | | QKPGQAPRLLIYGASRRATGIPDRFSGSGSGTDFT | |
| | | LTISRLEPEDSAVYYCQQYHSSPSWTFGQGTKLEI | |
| | | K | |
| BCMA- | VH | QVQLVESGGGLVQPGRSLRLSCAASGFTFDDYAMH | 269 |
| 3 | | WVRQAPGKGLEWVSGISWNSGSIGYADSVKGRFTI | |
| | | SRDNAKNSLYLQMNSLRAEDTALYYCSVHSFLAYW | |
| | | GQGTLVTVSS | |
| | VL | DIVMTQTPLSLPVTPGEPASISCRSSQSLLHSNGY | 270 |
| | | NYLDWYLQKPGQSPQLLIYLGSNRASGVPDRFSGS | |
| | | GSGTDFTLKISRVEAEDVGVYYCMQALQTPYTFGQ | |
| | | GTKVEIK | |
| | scFv | QVQLVESGGGLVQPGRSLRLSCAASGFTFDDYAMH | 271 |
| | | WVRQAPGKGLEWVSGISWNSGSIGYADSVKGRFTI | |
| | | SRDNAKNSLYLQMNSLRAEDTALYYCSVHSFLAYW | |
| | | GQGTLVTVSSASGGGGSGGRASGGGGSDIVMTQTP | |
| | | LSLPVTPGEPASISCRSSQSLLHSNGYNYLDWYLQ | |
| | | KPGQSPQLLIYLGSNRASGVPDRFSGSGSGTDFTL | |
| | | KISRVEAEDVGVYYCMQALQTPYTFGQGTKVEIK | |
| BCMA- | VH | EVQLLESGGGLVQPGGSLRLSCAVSGFALSNHGMS | 272 |
| 4 | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSS | |
| | VL | DIVMTQTPLSLSVTPGQPASISCKSSQSLLRNDGK | 273 |
| | | TPLYWYLQKAGQPPQLLIYEVSNRFSGVPDRFSGS | |
| | | GSGTDFTLKISRVEAEDVGAYYCMQNIQFPSFGGG | |
| | | TKLEIK | |
| | scFv | EVQLLESGGGLVQPGGSLRLSCAVSGFALSNHGMS | 274 |
| | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSSASGGGGSGGRASGGGGSDIVMTQTP | |
| | | LSLSVTPGQPASISCKSSQSLLRNDGKTPLYWYLQ | |
| | | KAGQPPQLLIYEVSNRFSGVPDRFSGSGSGTDFTL | |
| | | KISRVEAEDVGAYYCMQNIQFPSFGGGTKLEIK | |
| BCMA- | VH | QVQLVQSGAEVRKTGASVKVSCKASGYIFDNFGIN | 275 |
| 5 | | WVRQAPGQGLEWMGWINPKNNNTNYAQKFQGRVTI | |
| | | TADESTNTAYMEVSSLRSEDTAVYYCARGPYYYQS | |
| | | YMDVWGQGTMVTVSS | |
| | VL | DIVMTQTPLSLPVTPGEPASISCRSSQSLLHSNGY | 276 |
| | | NYLNWYLQKPGQSPQLLIYLGSKRASGVPDRFSGS | |
| | | GSGTDFTLHITRVGAEDVGVYYCMQALQTPYTFGQ | |
| | | GTKLEIK | |
| | scFv | QVQLVQSGAEVRKTGASVKVSCKASGYIFDNFGIN | 277 |
| | | WVRQAPGQGLEWMGWINPKNNNTNYAQKFQGRVTI | |
| | | TADESTNTAYMEVSSLRSEDTAVYYCARGPYYYQS | |
| | | YMDVWGQGTMVTVSSASGGGGSGGRASGGGGSDIV | |
| | | MTQTPLSLPVTPGEPASISCRSSQSLLHSNGYNYL | |
| | | NWYLQKPGQSPQLLIYLGSKRASGVPDRFSGSGSG | |
| | | TDFTLHITRVGAEDVGVYYCMQALQTPYTFGQGTK | |
| | | LEIK | |
| BCMA- | VH | QVQLQESGGGLVQPGGSLRLSCAASGFTFSSDAMT | 278 |
| 6 | | WVRQAPGKGLEQVSVISGSGGTTYYADSVKGRFTI | |
| | | SRDNSKNTLYLQMNSLRAEDTAVYYCAKLDSSGYY | |
| | | YARGPRYWGQGTLVTVSS | |
| | VL | DIQLTQSPSSLSASVGDRVTITCRASQSISSYLNW | 279 |
| | | YQQKPGKAPKLLIYGASTLASGVPARFSGSGSGTH | |
| | | FTLTINSLQSEDSATYYCQQSYKRASFGQGTKVEI | |
| | | K | |
| | scFv | QVQLQESGGGLVQPGGSLRLSCAASGFTFSSDAMT | 280 |
| | | WVRQAPGKGLEWVSVISGSGGTTYYADSVKGRFTI | |
| | | SRDNSKNTLYLQMNSLRAEDTAVYYCAKLDSSGYY | |
| | | YARGPRYWGQGTLVTVSSASGGGGSGGRASGGGGS | |
| | | DIQLTQSPSSLSASVGDRVTITCRASQSISSYLNW | |
| | | YQQKPGKAPKLLIYGASTLASGVPARFSGSGSGTH | |
| | | FTLTINSLQSEDSATYYCQQSYKRASFGQGTKVEI | |
| | | K | |
| BCMA- | VH | QVQLVQSGAEVKKPGASVKVSCKASGYTFSNYGIT | 281 |
| 7 | | WVRQAPGQGLEWMGWISAYNGNTNYAQKFQGRVTM | |
| | | TRNTSISTAYMELSSLRSEDTAVYYCARGPYYYYM | |
| | | DVWGKGTMVTVSS | |
| | VL | EIVMTQSPLSLPVTPGEPASISCRSSQSLLYSNGY | 282 |
| | | NYVDWYLQKPGQSPQLLIYLGSNRASGVPDRFSGS | |
| | | GSGTDFKLQISRVEAEDVGIYYCMQGRQFPYSFGQ | |
| | | GTKVEIK | |
| | scFv | QVQLVQSGAEVKKPGASVKVSCKASGYTFSNYGIT | 283 |
| | | WVRQAPGQGLEWMGWISAYNGNTNYAQKFQGRVTM | |
| | | TRNTSISTAYMELSSLRSEDTAVYYCARGPYYYYM | |
| | | DVWGKGTMVTVSSASGGGGSGGRASGGGGSEIVMT | |
| | | QSPLSLPVTPGEPASISCRSSQSLLYSNGYNYVDW | |
| | | YLQKPGQSPQLLIYLGSNRASGVPDRFSGSGSGTD | |
| | | FKLQISRVEAEDVGIYYCMQGRQFPYSFGQGTKVE | |
| | | IK | |
| BCMA- | VH | EVQLLETGGGLVQPGGSLRLSCAVSGFALSNHGMS | 284 |
| 8 | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSS | |
| | VL | EIVLTQSPATLSVSPGESATLSCRASQSVSSNLAW | 285 |
| | | YQQKPGQAPRLLIYGASTRASGIPDRFSGSGSGTD | |
| | | FTLTISSLQAEDVAVYYCQQYGSSLTFGGGTKVEI | |
| | | K | |
| | scFv | EVQLLETGGGLVQPGGSLRLSCAVSGFALSNHGMS | 286 |
| | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSSASGGGGSGGRASGGGGSEIVLTQSP | |
| | | ATLSVSPGESATLSCRASQSVSSNLAWYQQKPGQA | |
| | | PRLLIYGASTRASGIPDRFSGSGSGTDFTLTISSL | |
| | | QAEDVAVYYCQQYGSSLTFGGGTKVEIK | |
| BCMA- | VH | EVQLVETGGGLVQPGGSLRLSCAVSGFALSNHGMS | 287 |
| 9 | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSS | |
| | VL | EIVMTQSPATLSVSPGERATLSCRASQSVSSKLAW | 288 |
| | | YQQKPGQAPRLLMYGASIRATGIPDRFSGSGSGTE | |
| | | FTLTISSLEPEDFAVYYCQQYGSSSWTFGQGTKVE | |
| | | IK | |
| | scFv | EVQLVETGGGLVQPGGSLRLSCAVSGFALSNHGMS | 289 |
| | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSSASGGGGSGGRASGGGGSEIVMTQSP | |
| | | ATLSVSPGERATLSCRASQSVSSKLAWYQQKPGQA | |
| | | PRLLMYGASIRATGIPDRFSGSGSGTEFTLTISSL | |
| | | EPEDFAVYYCQQYGSSSWTFGQGTKVEIK | |
| BCMA- | VH | EVQLVETGGGVVQPGGSLRLSCAVSGFALSNHGMS | 290 |
| 10 | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSS | |
| | VL | EIVLTQSPGTLSLSPGERATLSCRASQSVGSTNLA | 291 |
| | | WYQQKPGQAPRLLIYDASNRATGIPDRFSGGGSGT | |
| | | DFTLTISRLEPEDFAVYYCQQYGSSPPWTFGQGTK | |
| | | VEIK | |
| | scFv | EVQLVETGGGVVQPGGSLRLSCAVSGFALSNHGMS | 292 |
| | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSSASGGGGSGGRASGGGGSEIVLTQSP | |
| | | GTLSLSPGERATLSCRASQSVGSTNLAWYQQKPGQ | |
| | | APRLLIYDASNRATGIPDRFSGGGSGTDFTLTISR | |
| | | LEPEDFAVYYCQQYGSSPPWTFGQGTKVEIK | |
| BCMA- | VH | QVQLVESGGGLVKPGGSLRLSCAASGFTFSDYYMS | 293 |
| 11 | | WIRQAPGKGLEWVSYISSSGSTIYYADSVKGRFTI | |
| | | SRDNAKNSLYLQMNSLRAEDTAVYYCARESGDGMD | |
| | | VWGQGTTVTVSS | |
| | VL | DIQMTQSPSSLSASVGDRVTITCRASQSISSYLNW | 294 |
| | | YQQKPGKAPKLLIYAASSLQSGVPSRFSGSGSGTD | |
| | | FTLTISSLQPEDFATYYCQQSYTLAFGQGTKVDIK | |
| | scFv | QVQLVESGGGLVKPGGSLRLSCAASGFTFSDYYMS | 295 |
| | | WIRQAPGKGLEWVSYISSSGSTIYYADSVKGRFTI | |
| | | SRDNAKNSLYLQMNSLRAEDTAVYYCARESGDGMD | |
| | | VWGQGTTVTVSSASGGGGSGGRASGGGGSDIQMTQ | |
| | | SPSSLSASVGDRVTITCRASQSISSYLNWYQQKPG | |
| | | KAPKLLIYAASSLQSGVPSRFSGSGSGTDFTLTIS | |
| | | SLQPEDFATYYCQQSYTLAFGQGTKVDIK | |
| BCMA- | VH | QVQLVQSGGGLVKPGGSLRLSCAASGFTFSDYYMS | 296 |
| 12 | | WIRQAPGKGLEWVSYISSSGNTIYYADSVKGRFTI | |
| | | SRDNAKNSLYLQMNSLRAEDTAVYYCARSTMVRED | |
| | | YWGQGTLVTVSS | |
| | VL | DIVLTQSPLSLPVTLGQPASISCKSSESLVHNSGK | 297 |
| | | TYLNWFHQRPGQSPRRLIYEVSNRDSGVPDRFTGS | |
| | | GSGTDFTLKISRVEAEDVGVYYCMQGTHWPGTFGQ | |
| | | GTKLEIK | |
| | scFv | QVQLVQSGGGLVKPGGSLRLSCAASGFTFSDYYMS | 298 |
| | | WIRQAPGKGLEWVSYISSSGNTIYYADSVKGRFTI | |
| | | SRDNAKNSLYLQMNSLRAEDTAVYYCARSTMVRED | |
| | | YWGQGTLVTVSSASGGGGSGGRASGGGGSDIVLTQ | |
| | | SPLSLPVTLGQPASISCKSSESLVHNSGKTYLNWF | |
| | | HQRPGQSPRRLIYEVSNRDSGVPDRFTGSGSGTDF | |
| | | TLKISRVEAEDVGVYYCMQGTHWPGTFGQGTKLEI | |
| | | K | |
| BCMA- | VH | QVQLVESGGGLVQPGGSLRLSCAVSGFALSNHGMS | 299 |
| 13 | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSS | |
| | VL | DIRLTQSPSPLSASVGDRVTITCQASEDINKFLNW | 300 |
| | | YHQTPGKAPKLLIYDASTLQTGVPSRFSGSGSGTD | |
| | | FTLTINSLQPEDIGTYYCQQYESLPLTFGGGTKVE | |
| | | IK | |
| | scFv | QVQLVESGGGLVQPGGSLRLSCAVSGFALSNHGMS | 301 |
| | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSSASGGGGSGGRASGGGGSDIRLTQSP | |
| | | SPLSASVGDRVTITCQASEDINKFLNWYHQTPGKA | |
| | | PKLLIYDASTLQTGVPSRFSGSGSGTDFTLTINSL | |
| | | QPEDIGTYYCQQYESLPLTFGGGTKVEIK | |
| BCMA- | VH | EVQLVETGGGLVQPGGSLRLSCAVSGFALSNHGMS | 287 |
| 14 | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSS | |
| | VL | ETTLTQSPATLSVSPGERATLSCRASQSVGSNLAW | 302 |
| | | YQQKPGQGPRLLIYGASTRATGIPARFSGSGSGTE | |
| | | FTLTISSLQPEDFAVYYCQQYNDWLPVTFGQGTKV | |
| | | EIK | |
| | scFv | EVQLVETGGGLVQPGGSLRLSCAVSGFALSNHGMS | 303 |
| | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSSASGGGGSGGRASGGGGSETTLTQSP | |
| | | ATLSVSPGERATLSCRASQSVGSNLAWYQQKPGQG | |
| | | PRLLIYGASTRATGIPARFSGSGSGTEFTLTISSL | |
| | | QPEDFAVYYCQQYNDWLPVTFGQGTKVEIK | |
| BCMA- | VH | EVQLVESGGGLVQPGGSLRLSCAVSGFALSNHGMS | 263 |
| 15 | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSS | |
| | VL | EIVLTQSPGTLSLSPGERATLSCRASQSIGSSSLA | 304 |
| | | WYQQKPGQAPRLLMYGASSRASGIPDRFSGSGSGT | |
| | | DFTLTISRLEPEDFAVYYCQQYAGSPPFTFGQGTK | |
| | | VEIK | |
| | scFv | EVQLVESGGGLVQPGGSLRLSCAVSGFALSNHGMS | 305 |
| | | WVRRAPGKGLEWVSGIVYSGSTYYAASVKGRFTIS | |
| | | RDNSRNTLYLQMNSLRPEDTAIYYCSAHGGESDVW | |
| | | GQGTTVTVSSASGGGGSGGRASGGGGSEIVLTQSP | |
| | | GTLSLSPGERATLSCRASQSIGSSSLAWYQQKPGQ | |
| | | APRLLMYGASSRASGIPDRFSGSGSGTDFTLTISR | |
| | | LEPEDFAVYYCQQYAGSPPFTFGQGTKVEIK | |
| BCMA- | VH | QVQLQESGPGLVKPSETLSLTCTVSGGSISSSYYY | 306 |
| 16 | | WGWIRQPPGKGLEWIGSIYYSGSAYYNPSLKSRVT | |
| | | ISVDTSKNQFSLRLSSVTAADTAVYYCARHWQEWP | |
| | | DAFDIWGQGTMVTVSS | |
| | VL | ETTLTQSPAFMSATPGDKVIISCKASQDIDDAMNW | 307 |
| | | YQQKPGEAPLFIIQSATSPVPGIPPRFSGSGFGTD | |
| | | FSLTINNIESEDAAYYFCLQHDNFPLTFGQGTKLE | |
| | | IK | |
| | scFv | QVQLQESGPGLVKPSETLSLTCTVSGGSISSSYYY | 308 |
| | | WGWIRQPPGKGLEWIGSIYYSGSAYYNPSLKSRVT | |
| | | ISVDTSKNQFSLRLSSVTAADTAVYYCARHWQEWP | |
| | | DAFDIWGQGTMVTVSSGGGGSGGGGSGGGGSETTL | |
| | | TQSPAFMSATPGDKVIISCKASQDIDDAMNWYQQK | |
| | | PGEAPLFIIQSATSPVPGIPPRFSGSGFGTDFSLT | |
| | | INNIESEDAAYYFCLQHDNFPLTFGQGTKLEIK | |
| BCMA- | VH | QVNLRESGPALVKPTQTLTLTCTFSGFSLRTSGMC | 309 |
| 17 | | VSWIRQPPGKALEWLARIDWDEDKFYSTSLKTRLT | |
| | | ISKDTSDNQVVLRMTNMDPADTATYYCARSGAGGT | |
| | | SATAFDIWGPGTMVTVSS | |
| | VL | DIQMTQSPSSLSASVGDRVTITCRASQDIYNNLAW | 310 |
| | | FQLKPGSAPRSLMYAANKSQSGVPSRFSGSASGTD | |
| | | FTLTISSLQPEDFATYYCQHYYRFPYSFGQGTKLE | |
| | | IK | |
| | scFv | VNLRESGPALVKPTQTLTLTCTFSGFSLRTSGMCV | 311 |
| | | SWIRQPPGKALEWLARIDWDEDKFYSTSLKTRLTI | |
| | | SKDTSDNQVVLRMTNMDPADTATYYCARSGAGGTS | |
| | | ATAFDIWGPGTMVTVSSGGGGSGGGGSGGGGSDIQ | |
| | | MTQSPSSLSASVGDRVTITCRASQDIYNNLAWFQL | |
| | | KPGSAPRSLMYAANKSQSGVPSRFSGSASGTDFTL | |
| | | TISSLQPEDFATYYCQHYYRFPYSFGQGTKLEIK | |
| BCMA- | VH | EVQLVESGGGLVKPGGSLRLSCAASGFTFSSYSMN | 312 |
| 18 | | WVRQAPGKGLEWVSSISSSSSYIYYADSVKGRFTI | |
| | | SRDNAKNSLYLQMNSLRAEDTAVYYCAKTIAAVYA | |
| | | FDIWGQGTTVTVSS | |
| | VL | EIVLTQSPLSLPVTPEEPASISCRSSQSLLHSNGY | 313 |
| | | NYLDWYLQKPGQSPQLLIYLGSNRASGVPDRFSGS | |
| | | GSGTDFTLKISRVEAEDVGVYYCMQALQTPYTFGQ | |
| | | GTKLEIK | |
| | scFv | EVQLVESGGGLVKPGGSLRLSCAASGFTFSSYSMN | 314 |
| | | WVRQAPGKGLEWVSSISSSSSYIYYADSVKGRFTI | |
| | | SRDNAKNSLYLQMNSLRAEDTAVYYCAKTIAAVYA | |
| | | FDIWGQGTTVTVSSGGGGSGGGGSGGGGSEIVLTQ | |
| | | SPLSLPVTPEEPASISCRSSQSLLHSNGYNYLDWY | |
| | | LQKPGQSPQLLIYLGSNRASGVPDRFSGSGSGTDF | |
| | | TLKISRVEAEDVGVYYCMQALQTPYTFGQGTKLEI | |
| | | K | |
| BCMA- | VH | EVQLVESGGGLVKPGGSLRLSCAASGFTFSDYYMS | 315 |
| 19 | | WIRQAPGKGLEWVSYISSSGSTIYYADSVKGRFTI | |
| | | SRDNAKNSLYLQMNSLRAEDTAVYYCARDLRGAFD | |
| | | IWGQGTMVTVSS | |
| | VL | SYVLTQSPSVSAAPGYTATISCGGNNIGTKSVHWY | 316 |
| | | QQKPGQAPLLVIRDDSVRPSKIPGRFSGSNSGNMA | |
| | | TLTISGVQAGDEADFYCQVWDSDSEHVVFGGGTKL | |
| | | TVL | |
| | scFv | EVQLVESGGGLVKPGGSLRLSCAASGFTFSDYYMS | 317 |
| | | WIRQAPGKGLEWVSYISSSGSTIYYADSVKGRFTI | |
| | | SRDNAKNSLYLQMNSLRAEDTAVYYCARDLRGAFD | |
| | | IWGQGTMVTVSSGGGGSGGGGSGGGGSSYVLTQSP | |
| | | SVSAAPGYTATISCGGNNIGTKSVHWYQQKPGQAP | |
| | | LLVIRDDSVRPSKIPGRFSGSNSGNMATLTISGVQ | |
| | | AGDEADFYCQVWDSDSEHVVFGGGTKLTVL | |
| BCMA- | VH | QVQLVQSGAEVKKPGASVKVSCKPSGYTVTSHYIH | 318 |
| 20 | | WVRRAPGQGLEWMGMINPSGGVTAYSQTLQGRVTM | |
| | | TSDTSSSTVYMELSSLRSEDTAMYYCAREGSGSGW | |
| | | YFDFWGRGTLVTVSS | |
| | VL | SYVLTQPPSVSVSPGQTASITCSGDGLSKKYVSWY | 319 |
| | | QQKAGQSPVVLISRDKERPSGIPDRFSGSNSADTA | |
| | | TLTISGTQAMDEADYYCQAWDDTTVVFGGGTKLTV | |
| | | L | |
| | scFv | QVQLVQSGAEVKKPGASVKVSCKPSGYTVTSHYIH | 320 |
| | | WVRRAPGQGLEWMGMINPSGGVTAYSQTLQGRVTM | |
| | | TSDTSSSTVYMELSSLRSEDTAMYYCAREGSGSGW | |
| | | YFDFWGRGTLVTVSSGGGGSGGGGSGGGGSSYVLT | |
| | | QPPSVSVSPGQTASITCSGDGLSKKYVSWYQQKAG | |
| | | QSPVVLISRDKERPSGIPDRFSGSNSADTATLTIS | |
| | | GTQAMDEADYYCQAWDDTTVVFGGGTKLTVL | |
| BCMA- | VH | QVQLQESGPGLVKPSQTLSLTCTVSGGSISSGGYY | 321 |
| 21 | | WSWIRQHPGKGLEWIGYIYYSGSTYYNPSLKSRVT | |
| | | ISVDTSKNQFSLKLSSVTAADTAVYYCARAGIAAR | |
| | | LRGAFDIWGQGTMVTVSS | |
| | VL | DIVMTQSPSSVSASVGDRVIITCRASQGIRNWLAW | 322 |
| | | YQQKPGKAPNLLIYAASNLQSGVPSRFSGSGSGAD | |
| | | FTLTISSLQPEDVATYYCQKYNSAPFTFGPGTKVD | |
| | | IK | |
| | scFv | QVQLQESGPGLVKPSQTLSLTCTVSGGSISSGGYY | 323 |
| | | WSWIRQHPGKGLEWIGYIYYSGSTYYNPSLKSRVT | |
| | | ISVDTSKNQFSLKLSSVTAADTAVYYCARAGIAAR | |
| | | LRGAFDIWGQGTMVTVSSGGGGSGGGGSGGGGSDI | |
| | | VMTQSPSSVSASVGDRVIITCRASQGIRNWLAWYQ | |
| | | QKPGKAPNLLIYAASNLQSGVPSRFSGSGSGADFT | |
| | | LTISSLQPEDVATYYCQKYNSAPFTFGPGTKVDIK | |
| BCMA- | VH | QVQLVQSGAEVKKPGSSVKVSCKASGGTFSSYAIS | 324 |
| 22 | | WVRQAPGQGLEWMGGIIPIFGTANYAQKFQGRVTI | |
| | | TADESTSTAYMELSSLRSEDTAVYYCARRGGYQLL | |
| | | RWDVGLLRSAFDIWGQGTMVTVSS | |
| | VL | SYVLTQPPSVSVAPGQTARITCGGNNIGSKSVHWY | 325 |
| | | QQKPGQAPVLVLYGKNNRPSGVPDRFSGSRSGTTA | |
| | | SLTITGAQAEDEADYYCSSRDSSGDHLRVFGTGTK | |
| | | VTVL | |
| | scFv | QVQLVQSGAEVKKPGSSVKVSCKASGGTFSSYAIS | 326 |
| | | WVRQAPGQGLEWMGGIIPIFGTANYAQKFQGRVTI | |
| | | TADESTSTAYMELSSLRSEDTAVYYCARRGGYQLL | |
| | | RWDVGLLRSAFDIWGQGTMVTVSSGGGGSGGGGSG | |
| | | GGGSSYVLTQPPSVSVAPGQTARITCGGNNIGSKS | |
| | | VHWYQQKPGQAPVLVLYGKNNRPSGVPDRFSGSRS | |
| | | GTTASLTITGAQAEDEADYYCSSRDSSGDHLRVFG | |
| | | TGTKVTVL | |
| BCMA- | VH | EVQLQQSGPGLVKPSQTLSLTCAISGDSVSSNSAA | 327 |
| 23 | | WNWIRQSPSRGLEWLGRTYYRSKWYSFYAISLKSR | |
| | | IIINPDTSKNQFSLQLKSVTPEDTAVYYCARSSPE | |
| | | GLFLYWFDPWGQGTLVTVSS | |
| | VL | SSELTQDPAVSVALGQTIRITCQGDSLGNYYATWY | 328 |
| | | QQKPGQAPVLVIYGTNNRPSGIPDRFSASSSGNTA | |
| | | SLTITGAQAEDEADYYCNSRDSSGHHLLFGTGTKV | |
| | | TVL | |
| | ScFv | EVQLQQSGPGLVKPSQTLSLTCAISGDSVSSNSAA | 329 |
| | | WNWIRQSPSRGLEWLGRTYYRSKWYSFYAISLKSR | |
| | | IIINPDTSKNQFSLQLKSVTPEDTAVYYCARSSPE | |
| | | GLFLYWFDPWGQGTLVTVSSGGDGSGGGGSGGGGS | |
| | | SSELTQDPAVSVALGQTIRITCQGDSLGNYYATWY | |
| | | QQKPGQAPVLVIYGTNNRPSGIPDRFSASSSGNTA | |
| | | SLTITGAQAEDEADYYCNSRDSSGHHLLFGTGTKV | |
| | | TVL | |
| BCMA- | VH | EVQLVESGGGLVQPGGSLRLSCAASGFTFSSYAMS | 330 |
| 24 | | WVRQAPGKGLEQVSAISGSGGSTYYADSVKGRFTI | |
| | | SRDNSKNTLYLQMNSLRAEDTAVYYCAKVEGSGSL | |
| | | DYWGQGTLVTVSS | |
| | VL | EIVMTQSPGTLSLSPGERATLSCRASQSVSSAYLA | 331 |
| | | WYQQKPGQPPRLLISGASTRATGIPDRFGGSGSGT | |
| | | DFTLTISRLEPEDFAVYYCQHYGSSFNGSSLFTFG | |
| | | QGTRLEIK | |
| | scFv | EVQLVESGGGLVQPGGSLRLSCAASGFTFSSYAMS | 332 |
| | | WVRQAPGKGLEQVSAISGSGGSTYYADSVKGRFTI | |
| | | SRDNSKNTLYLQMNSLRAEDTAVYYCAKVEGSGSL | |
| | | DYWGQGTLVTVSSGGGGSGGGGSGGGGSEIVMTQS | |
| | | PGTLSLSPGERATLSCRASQSVSSAYLAWYQQKPG | |
| | | QPPRLLISGASTRATGIPDRFGGSGSGTDFTLTIS | |
| | | RLEPEDFAVYYCQHYGSSFNGSSLFTFGQGTRLEI | |
| | | K | |
| | VH | EVQLVETGGGLVQPGGSLRLSCAASGITFSRYPMS | 333 |
| | | QVRQAPGKGLEWVSGISDSGVSTYYADSAKGRFTI | |
| | | SRDNSKNTLFLQMSSLRDEDTAVYYCVTRAGSEAS | |
| | | DIWGQGTMVTVSS | |
| BCMA- | VL | EIVLTQSPATLSLSPGERATLSCRASQSVSNSLAW | 334 |
| 25 | | YQQKPGQAPRLLIYDASSRATGIPDRFSGSGSGTD | |
| | | FTLTISRLEPEDFAIYYCQQFGTSSGLTFGGGTKL | |
| | | EIK | |
| | scFv | EVQLVETGGGLVQPGGSLRLSCAASGITFSRYPMS | 335 |
| | | WVRQAPGKGLEWVSGISDSGVSTYYADSAKGRFTI | |
| | | SRDNSKNTLFLQMSSLRDEDTAVYYCVTRAGSEAS | |
| | | DIWGQGTMVTVSSGGGGSGGGGSGGGGSEIVLTQS | |
| | | PATLSLSPGERATLSCRASQSVSNSLAWYQQKPGQ | |
| | | APRLLIYDASSRATGIPDRFSGSGSGTDFTLTISR | |
| | | LEPEDFAIYYCQQFGTSSGLTFGGGTKLEIK | |
| BCMA- | VH | QVQLVESGGGLVQPGGSLRLSCAASGFTFSSYAMS | 336 |
| 26 | | WVRQAPGKGLEWVSAISGSGGSTYYADSVKGRFTI | |
| | | SRDNAKNSLYLQMNSLRAEDTAIYYCARATYKREL | |
| | | RYYYGMDVWGQGTMVTVSS | |
| | VL | EIVMTQSPGTVSLSPGERATLSCRASQSVSSSFLA | 337 |
| | | WYQQKPGQAPRLLIYGASSRATGIPDRFSGSGSGT | |
| | | DFTLTISRLEPEDSAVYYCQQYHSSPSWTFGQGTR | |
| | | LEIK | |
| | scFv | QVQLVESGGGLVQPGGSLRLSCAASGFTFSSYAMS | 338 |
| | | WVRQAPGKGLEWVSAISGSGGSTYYADSVKGRFTI | |
| | | SRDNAKNSLYLQMNSLRAEDTAIYYCARATYKREL | |
| | | RYYYGMDVWGQGTMVTVSSGGGGSGGGGSGGGGSE | |
| | | IVMTQSPGTVSLSPGERATLSCRASQSVSSSFLAW | |
| | | YQQKPGQAPRLLIYGASSRATGIPDRFSGSGSGTD | |
| | | FTLTISRLEPEDSAVYYCQQYHSSPSWTFGQGTRL | |
| | | EIK | |
| BCMA- | VH | EVQLVETGGGLVQPGGSLRLSCAASGFTFSSYAMS | 339 |
| 27 | | WVRQAPGKGLEWVSAISGSGGSTYYADSVKGRFTI | |
| | | SRDNSKNTLYLQMNTLKAEDTAVYYCARATYKREL | |
| | | RYYYGMDVWGQGTTVTVSS | |
| | VL | EIVLTQSPSTLSLSPGESATLSCRASQSVSTTFLA | 340 |
| | | WYQQKPGQAPRLLIYGSSNRATGIPDRFSGSGSGT | |
| | | DFTLTIRRLEPEDFAVYYCQQYHSSPSWTFGQGTK | |
| | | VEIK | |
| | scFv | EVQLVETGGGLVQPGGSLRLSCAASGFTFSSYAMS | 341 |
| | | WVRQAPGKGLEWVSAISGSGGSTYYADSVKGRFTI | |
| | | SRDNSKNTLYLQMNTLKAEDTAVYYCARATYKREL | |
| | | RYYYGMDVWGQGTTVTVSSGGGGSGGGGSGGGGSE | |
| | | IVLTQSPSTLSLSPGESATLSCRASQSVSTTFLAW | |
| | | YQQKPGQAPRLLIYGSSNRATGIPDRFSGSGSGTD | |
| | | FTLTIRRLEPEDFAVYYCQQYHSSPSWTFGQGTKV | |
| | | EIK | |
| BCMA- | VH | EVQLVETGGGLVQPGRSLRLSCAASGFTFDDYAMH | 342 |
| 28 | | WVRQAPGKGLEWVSGISWNSGSIGYADSVKGRFTI | |
| | | SRDNAKNSLYLQMNSLRDEDTAVYYCARVGKAVPD | |
| | | VWGQGTTVTVSS | |
| | VL | DIVMTQTPSSLSASVGDRVTITCRASQSISSYLNW | 343 |
| | | YQQKPGKAPKLLIYAASSLQSGVPSRFSGSGSGTD | |
| | | FTLTISSLQPEDFATYYCQQSYSTPYSFGQGTRLE | |
| | | IK | |
| | scFv | EVQLVETGGGLVQPGRSLRLSCAASGFTFDDYAMH | 344 |
| | | WVRQAPGKGLEWVSGISWNSGSIGYADSVKGRFTI | |
| | | SRDNAKNSLYLQMNSLRDEDTAVYYCARVGKAVPD | |
| | | VWGQGTTVTVSSGGGGSGGGGSGGGGSDIVMTQTP | |
| | | SSLSASVGDRVTITCRASQSISSYLNWYQQKPGKA | |
| | | PKLLIYAASSLQSGVPSRFSGSGSGTDFTLTISSL | |
| | | QPEDFATYYCQQSYSTPYSFGQGTRLEIK | |
| BCMA- | VH | EVQLVESGGGLVQPGRSLRLSCTASGFTFDDYAMH | 345 |
| 29 | | WVRQRPGKGLWEWVASINWKGNSLAYGDSVKGRFA | |
| | | ISRDNAKNTVFLQMNSLRTEDTAVYYCASHQGVAY | |
| | | YNYAMDVWGRGTLVTVSS | |
| | VL | EIVLTQSPGTLSLSPGERATLSCRATQSIGSSFLA | 346 |
| | | WYQQRPGQAPRLLIYGASQRATGIPDRFSGRGSGT | |
| | | DFTLTISRVEPEDSAVYYCQHYESSPSWTFGQGTK | |
| | | VEIK | |
| | scFv | EVQLVESGGGLVQPGRSLRLSCTASGFTFDDYAMH | 347 |
| | | WVRQRPGKGLEWVASINWKGNSLAYGDSVKGRFAI | |
| | | SRDNAKNTVFLQMNSLRTEDTAVYYCASHQGVAYY | |
| | | NYAMDVWGRGTLVTVSSGGGGSGGGGSGGGGSEIV | |
| | | LTQSPGTLSLSPGERATLSCRATQSIGSSFLAWYQ | |
| | | QRPGQAPRLLIYGASQRATGIPDRFSGRGSGTDFT | |
| | | LTISRVEPEDSAVYYCQHYESSPSWTFGQGTKVEI | |
| | | K | |
| BCMA- | VH | EVQLVESGGGLVQPGGSLRLSCAASGFTFSSYAMS | 348 |
| 30 | | WVRQAPGKGLEWVSAISGSGGSTYYADSVKGRFTI | |
| | | SRDNSKNTLYLQMNSLRAEDTAVYYCAKVVRDGMD | |
| | | VWGQGTTVTVSS | |
| | VL | EIVLTQSPATLSLSPGERATLSCRASQSVSSSYLA | 349 |
| | | WYQQKPGQAPRLLIYGASSRATGIPDRFSGNGSGT | |
| | | DFTLTISRLEPEDFAVYYCQQYGSPPRFTFGPGTK | |
| | | VDIK | |
| | scFv | EVQLVESGGGLVQPGGSLRLSCAASGFTFSSYAMS | 350 |
| | | WVRQAPGKGLEWVSAISGSGGSTYYADSVKGRFTI | |
| | | SRDNSKNTLYLQMNSLRAEDTAVYYCAKVVRDGMD | |
| | | VWGQGTTVTVSSGGGGSGGGGSGGGGSEIVLTQSP | |
| | | ATLSLSPGERATLSCRASQSVSSSYLAWYQQKPGQ | |
| | | APRLLIYGASSRATGIPDRFSGNGSGTDFTLTISR | |
| | | LEPEDFAVYYCQQYGSPPRFTFGPGTKVDIK | |
| BCMA- | VH | EVQLLESGGGLVQPGGSLRLSCAASGFTFSSYAMS | 351 |
| 31 | | WVRQAPGKGLEWVSAISGSGGSTYYADSVKGRFTI | |
| | | SRDNSKNTLYLQMNSLRAEDTAVYYCAKIPQTGTF | |
| | | DYWGQGTLVTVSS | |
| | VL | EIVLTQSPGTLSLSPGERATLSCRASQSVSSSYLA | 352 |
| | | WYQQRPGQAPRLLIYGASSRATGIPDRFSGSGSGT | |
| | | DFTLTISRLEPEDFAVYYCQHYGSSPSWTFGQGTR | |
| | | LEIK | |
| | scFv | EVQLLESGGGLVQPGGSLRLSCAASGFTFSSYAMS | 353 |
| | | WVRQAPGKGLEWVSAISGSGGSTYYADSVKGRFTI | |
| | | SRDNSKNTLYLQMNSLRAEDTAVYYCAKIPQTGTF | |
| | | DYWGQGTLVTVSSGGGGSGGGGSGGGGSEIVLTQS | |
| | | PGTLSLSPGERATLSCRASQSVSSSYLAWYQQRPG | |
| | | QAPRLLIYGASSRATGIPDRFSGSGSGTDFTLTIS | |
| | | RLEPEDFAVYYCQHYGSSPSWTFGQGTRLEIK | |
| BCMA- | VH | EVQLVETGGGLVQPGGSLRLSCAASGFTFSSYAMS | 354 |
| 32 | | WVRQAPGKGLEWVSAISGSGGSTYYADSVKGRFTM | |
| | | SRENDKNSVFLQMNSLRVEDTGVYYCARANYKREL | |
| | | RYYYGMDVWGQGTMVTVSS | |
| | VL | EIVMTQSPGTLSLSPGESATLSCRASQRVASNYLA | 355 |
| | | WYQHKPGQAPSLLISGASSRATGVPDRFSGSGSGT | |
| | | DFTLAISRLEPEDSAVYYCQHYDSSPSWTFGQGTK | |
| | | VEIK | |
| | scFv | EVQLVETGGGLVQPGGSLRLSCAASGFTFSSYAMS | 356 |
| | | WVRQAPGKGLEWVSAISGSGGSTYYADSVKGRFTM | |
| | | SRENDKNSVFLQMNSLRVEDTGVYYCARANYKREL | |
| | | RYYYGMDVWGQGTMVTVSSGGGGSGGGGSGGGGSE | |
| | | IVMTQSPGTLSLSPGESATLSCRASQRVASNYLAW | |
| | | YQHKPGQAPSLLISGASSRATGVPDRFSGSGSGTD | |
| | | FTLAISRLEPEDSAVYYCQHYDSSPSWTFGQGTKV | |
| | | EIK | |
| BCMA- | VH | EVQLLETGGGLVQPGGSLRLSCAASGFSFSSYAMS | 357 |
| 33 | | WVRQAPGKGLEWVSAISGSGGSTYYADSVKGRFTI | |
| | | SRDNSKNTLYLQMNSLRAEDTAVYYCAKALVGATG | |
| | | AFDIWGQGTLVTVSS | |
| | VL | EIVLTQSPGTLSLSPGERATLSCRASQSLSSNFLA | 358 |
| | | WYQQKPGQAPGLLIYGASNWATGTPDRFSGSGSGT | |
| | | DFTLTITRLEPEDFAVYYCQYYGTSPMYTFGQGTK | |
| | | VEIK | |
| | scFv | EVQLLETGGGLVQPGGSLRLSCAASGFSFSSYAMS | 359 |
| | | WVRQAPGKGLEWVSAISGSGGSTYYADSVKGRFTI | |
| | | SRDNSKNTLYLQMNSLRAEDTAVYYCAKALVGATG | |
| | | AFDIWGQGTLVTVSSGGGGSGGGGSGGGGSEIVLT | |
| | | QSPGTLSLSPGERATLSCRASQSLSSNFLAWYQQK | |
| | | PGQAPGLLIYGASNWATGTPDRFSGSGSGTDFTLT | |
| | | ITRLEPEDFAVYYCQYYGTSPMYTFGQGTKVEIK | |
| BCMA- | VH | EVQLLESGGGLVQPGGSLRLSCAASGFTFSSYAMS | 360 |
| 34 | | WVRQAPGKGLEWVSAISGSGGSTYYADSVKGRFTI | |
| | | SRDNSKNTLYLQMNSLRAEDTAVYYCVLWFGEGFD | |
| | | PWGQGTLVTVSS | |
| | VL | DIVLTQSPLSLPVTPGEPASISCRSSQSLLHSNGY | 361 |
| | | NYLDWYLQKPGQSPQLLIYLGSNRASGVPDRFSGS | |
| | | GSGTDFTLKISRVEAEDVGVYYCMQALQTPLTFGG | |
| | | GTKVDIK | |
| | scFv | EVQLLESGGGLVQPGGSLRLSCAASGFTFSSYAMS | 362 |
| | | WVRQAPGKGLEWVSAISGSGGSTYYADSVKGRFTI | |
| | | SRDNSKNTLYLQMNSLRAEDTAVYYCVLWFGEGFD | |
| | | PWGQGTLVTVSSGGGGSGGGGSGGGGSDIVLTQSP | |
| | | LSLPVTPGEPASISCRSSQSLLHSNGYNYLDWYLQ | |
| | | KPGQSPQLLIYLGSNRASGVPDRFSGSGSGTDFTL | |
| | | KISRVEAEDVGVYYCMQALQTPLTFGGGTKVDIK | |
| BCMA- | VH | QVQLVESGGGLVQPGGSLRLSCAASGFTFSSYAMS | 363 |
| 35 | | WVRQAPGKGLEWVSAISGSGGSTYYADSVKGRFTI | |
| | | SRDNSKNTLYLQMNSLRAEDTAVYYCAKVGYDSSG | |
| | | YYRDYYGMDVWGQGTTVTVSS | |
| | VL | EIVLTQSPGTLSLSPGERATLSCRASQSVSSSYLA | 364 |
| | | WYQQKPGQAPRLLIYGTSSRATGISDRFSGSGSGT | |
| | | DFTLTISRLEPEDFAVYYCQHYGNSPPKFTFGPGT | |
| | | KLEIK | |
| | scFv | QVQLVESGGGLVQPGGSLRLSCAASGFTFSSYAMS | 365 |
| | | WVRQAPGKGLEWVSAISGSGGSTYYADSVKGRFTI | |
| | | SRDNSKNTLYLQMNSLRAEDTAVYYCAKVGYDSSG | |
| | | YYRDYYGMDVWGQGTTVTVSSGGGGSGGGGSGGGG | |
| | | SEIVLTQSPGTLSLSPGERATLSCRASQSVSSSYL | |
| | | AWYQQKPGQAPRLLIYGTSSRATGISDRFSGSGSG | |
| | | TDFTLTISRLEPEDFAVYYCQHYGNSPPKFTFGPG | |
| | | TKLEIK | |
| BCMA- | VH | EVQLVESGGGLVQPGGSLRLSCAASGFTFSSYAMS | 366 |
| 36 | | WVRQAPGKGLEWVSAISGSGGSTYYADSVKGRFTI | |
| | | SRDNSKNTLYLQMNSLRAEDTAVYYCAKMGWSSGY | |
| | | LGAFDIWGQGTTVTVSS | |
| | VL | EIVLTQSPGTLSLSPGERATLSCRASQSVASSFLA | 367 |
| | | WYQQKPGQAPRLLIYGASGRATGIPDRFSGSGSGT | |
| | | DFTLTISRLEPEDFAVYYCQHYGGSPRLTFGGGTK | |
| | | VDIK | |
| | scFv | EVQLVESGGGLVQPGGSLRLSCAASGFTFSSYAMS | 368 |
| | | WVRQAPGKGLEWVSAISGSGGSTYYADSVKGRFTI | |
| | | SRDNSKNTLYLQMNSLRAEDTAVYYCAKMGWSSGY | |
| | | LGAFDIWGQGTTVTVSSGGGGSGGGGSGGGGSEIV | |
| | | LTQSPGTLSLSPGERATLSCRASQSVASSFLAWYQ | |
| | | QKPGQAPRLLIYGASGRATGIPDRFSGSGSGTDFT | |
| | | LTISRLEPEDFAVYYCQHYGGSPRLTFGGGTKVDI | |
| | | K | |
| BCMA- | VH | QIQLVQSGPDLKKPGETVKLSCKASGYTFTNFGMN | 369 |
| 37 | | WVKQAPGKGFKWMAWINTYTGESYFADDFKGRFAF | |
| | | SVETSATTAYLQINNLKTEDTATYFCARGEIYYGY | |
| | | DGGFAYWGQGTLVTVSA | |
| | VL | DVVMTQSHRFMSTSVGDRVSITCRASQDVNTAVSW | 370 |
| | | YQQKPGQSPKLLIFSASYRYTGVPDRFTGSGSGAD | |
| | | FTLTISSVQAEDLAVYYCQQHYSTPWTFGGGTKLD | |
| | | IK | |
| | scFv | QIQLVQSGPDLKKPGETVKLSCKASGYTFTNFGMN | 371 |
| | | WVKQAPGKGFKWMAWINTYTGESYFADDFKGRFAF | |
| | | SVETSATTAYLQINNLKTEDTATYFCARGEIYYGY | |
| | | DGGFAYWGQGTLVTVSAGGGGSGGGGSGGGGSDVV | |
| | | MTQSHRFMSTSVGDRVSITCRASQDVNTAVSWYQQ | |
| | | KPGQSPKLLIFSASYRYTGVPDRFTGSGSGADFTL | |
| | | TISSVQAEDLAVYYCQQHYSTPWTFGGGTKLDIK | |
| BCMA- | VH | QIQLVQSGPELKKPGETVKISCKASGYTFTDYSIN | 372 |
| 38 | | WVKRAPGKGLKWMGWINTETREPAYAYDFRGRFAF | |
| | | SLETSASTAYLQINNLKYEDTATYFCALDYSYAMD | |
| | | YWGQGTSVTVSS | |
| | VL | DIVLTQSPASLAMSLGKRATISCRASESVSVIGAH | 373 |
| | | LIHWYQQKPGQPPKLLIYLASNLETGVPARFSGSG | |
| | | SGTDFTLTIDPVEEDDVAIYSCLQSRIFPRTFGGG | |
| | | TKLEIK | |
| | scFv | QIQLVQSGPELKKPGETVKISCKASGYTFTDYSIN | 374 |
| | | WVKRAPGKGLKWMGWINTETREPAYAYDFRGRFAF | |
| | | SLETSASTAYLQINNLKYEDTATYFCALDYSYAMD | |
| | | YWGQGTSVTVSSGGGGSGGGGSGGGGSQIQLVQSG | |
| | | PELKKPGETVKISCKASGYTFTDYSINWVKRAPGK | |
| | | GLKWMGWINTETREPAYAYDFRGRFAFSLETSAST | |
| | | AYLQINNLKYEDTATYFCALDYSYAMDYWGQGTSV | |
| | | TVSS | |
| BCMA- | VH | QIQLVQSGPELKKPGETVKISCKASGYTFRHYSMN | 375 |
| 39 | | WVKQAPGKGLKWMGRINTESGVPIYADDFKGRFAF | |
| | | SVETSASTAYLVINNLKDEDTASYFCSNDYLYSLD | |
| | | FWGQGTALTVSS | |
| | VL | DIVLTQSPPSLAMSLGKRATISCRASESVTILGSH | 376 |
| | | LIYWYQQKPGQPPTLLIQLASNVQTGVPARFSGSG | |
| | | SRTDFTLTIDPVEEDDVAVYYCLQSRTIPRTFGGG | |
| | | TKLEIK | |
| | scFv | QIQLVQSGPELKKPGETVKISCKASGYTFRHYSMN | 377 |
| | | WVKQAPGKGLKWMGRINTESGVPIYADDFKGRFAF | |
| | | SVETSASTAYLVINNLKDEDTASYFCSNDYLYSLD | |
| | | FWGQGTALTVSSGGGGSGGGGSGGGGSDIVLTQSP | |
| | | PSLAMSLGKRATISCRASESVTILGSHLIYWYQQK | |
| | | PGQPPTLLIQLASNVQTGVPARFSGSGSRTDFTLT | |
| | | IDPVEEDDVAVYYCLQSRTIPRTFGGGTKLEIK | |
| BCMA- | VH | QIQLVQSGPELKKPGETVKISCKASGYTFTHYSMN | 378 |
| 40 | | WVKQAPGKGLKWMGRINTETGEPLYADDFKGRFAF | |
| | | SLETSASTAYLVINNLKNEDTATFFCSNDYLYSCD | |
| | | YWGQGTTLTVSS | |
| | VL | DIVLTQSPPSLAMSLGKRATISCRASESVTILGSH | 376 |
| | | LIYWYQQKPGQPPTLLIQLASNVQTGVPARFSGSG | |
| | | SRTDFTLTIDPVEEDDVAVYYCLQSRTIPRTFGGG | |
| | | TKLEIK | |
| | scFv | QIQLVQSGPELKKPGETVKISCKASGYTFTHYSMN | 379 |
| | | WVKQAPGKGLKWMGRINTETGEPLYADDFKGRFAF | |
| | | SLETSASTAYLVINNLKNEDTATFFCSNDYLYSCD | |
| | | YWGQGTTLTVSSGGGGSGGGGSGGGGSDIVLTQSP | |
| | | PSLAMSLGKRATISCRASESVTILGSHLIYWYQQK | |
| | | PGQPPTLLIQLASNVQTGVPARFSGSGSRTDFTLT | |
| | | IDPVEEDDVAVYYCLQSRTIPRTFGGGTKLEIK | |

TABLE 12B
BCMA Binders - Light chain CDR sequences
according to Kabat numbering scheme

| | | SEQ | | SEQ | | SEQ |
|---|---|---|---|---|---|---|
| Anti- | | ID | | ID | | ID |
| body | CDR-L1 | NO: | CDR-L2 | NO: | CDR-L3 | NO: |
| BCMA- | RASQSISSYLN | 380 | AASSLQS | 412 | QQSYSTPYT | 441 |
| 1 | | | | | | |
| BCMA- | RASQSISSSFLA | 381 | GASRRAT | 413 | QQYHSSPSWT | 442 |
| 2 | | | | | | |
| BCMA- | RSSQSLLHSNGY | 382 | LGSNRAS | 414 | MQALQTPYT | 443 |
| 3 | NYLD | | | | | |
| BCMA- | KSSQSLLRNDGK | 383 | EVSNRFS | 415 | MQNIQFPS | 444 |
| 4 | TPLY | | | | | |
| BCMA- | RSSQSLLHSNGY | 384 | LGSKRAS | 416 | MQALQTPYT | 443 |
| 5 | NYLN | | | | | |
| BCMA- | RASQSISSYLN | 380 | GASTLAS | 417 | QQSYKRAS | 445 |
| 6 | | | | | | |
| BCMA- | RSSQSLLYSNGY | 385 | LGSNRAS | 414 | MQGRQFPYS | 446 |
| 7 | NYVD | | | | | |
| BCMA- | RASQSVSSNLA | 386 | GASTRAS | 418 | QQYGSSLT | 447 |
| 8 | | | | | | |
| BCMA- | RASQSVSSKLA | 387 | GASIRAT | 419 | QQYGSSSWT | 448 |
| 9 | | | | | | |
| BCMA- | RASQSVGSTNLA | 388 | DASNRAT | 158 | QQYGSSPPWT | 449 |
| 10 | | | | | | |
| BCMA- | RASQSISSYLN | 380 | AASSLQS | 412 | QQSYTLA | 450 |
| 11 | | | | | | |
| BCMA- | KSSESLVHNSGK | 389 | EVSNRDS | 420 | MQGTHWPGT | 451 |
| 12 | TYLN | | | | | |
| BCMA- | QASEDINKFLN | 390 | DASTLQT | 421 | QQYESLPLT | 452 |
| 13 | | | | | | |
| BCMA- | RASQSVGSNLA | 391 | GASTRAT | 422 | QQYNDWLPVT | 453 |
| 14 | | | | | | |
| BCMA- | RASQSIGSSSLA | 392 | GASSRAS | 423 | QQYAGSPPFT | 454 |
| 15 | | | | | | |
| BCMA- | KASQDIDDAMN | 393 | SATSPVP | 424 | LQHDNFPLT | 455 |
| 16 | | | | | | |
| BCMA- | RASQDIYNNLA | 394 | AANKSQS | 425 | QHYYRFPYS | 456 |
| 17 | | | | | | |
| BCMA- | RSSQSLLHSNGY | 382 | LGSNRAS | 414 | MQALQTPYT | 443 |
| 18 | NYLD | | | | | |
| BCMA- | GGNNIGTKSVH | 395 | DDSVRPS | 426 | QVWDSDSEHV | 457 |
| 19 | | | | | V | |
| BCMA- | SGDGLSKKYVS | 396 | RDKERPS | 427 | QAWDDTTVV | 458 |
| 20 | | | | | | |
| BCMA- | RASQGIRNWLA | 397 | AASNLQS | 428 | QKYNSAPFT | 459 |
| 21 | | | | | | |
| BCMA- | GGNNIGSKSVH | 398 | GKNNRPS | 429 | SSRDSSGDHL | 460 |
| 22 | | | | | RV | |
| BCMA- | QGDSLGNYYAT | 399 | GTNNRPS | 430 | NSRDSSGHHL | 461 |
| 23 | | | | | L | |
| BCMA- | RASQSVSSAYLA | 400 | GASTRAT | 422 | QHYGSSFNGS | 462 |
| 24 | | | | | SLFT | |
| BCMA- | RASQSVSNSLA | 401 | DASSRAT | 431 | QQFGTSSGLT | 463 |
| 25 | | | | | | |
| BCMA- | RASQSVSSSFLA | 402 | GASSRAT | 432 | QQYHSSPSWT | 442 |
| 26 | | | | | | |
| BCMA- | RASQSVSTTFLA | 403 | GSSNRAT | 433 | QQYHSSPSWT | 442 |
| 27 | | | | | | |
| BCMA- | RASQSISSYLN | 380 | AASSLQS | 412 | QQSYSTPYS | 464 |
| 28 | | | | | | |
| BCMA- | RATQSIGSSFLA | 404 | GASQRAT | 434 | QHYESSPSWT | 465 |
| 29 | | | | | | |
| BCMA- | RASQSVSSSYLA | 405 | GASSRAT | 432 | QQYGSPPRFT | 466 |
| 30 | | | | | | |
| BCMA- | RASQSVSSSYLA | 405 | GASSRAT | 432 | QHYGSSPSWT | 467 |
| 31 | | | | | | |
| BCMA- | RASQRVASNYLA | 406 | GASSRAT | 432 | QHYDSSPSWT | 468 |
| 32 | | | | | | |
| BCMA- | RASQSLSSNFLA | 407 | GASNWAT | 435 | QYYGTSPMYT | 469 |
| 33 | | | | | | |
| BCMA- | RSSQSLLHSNGY | 382 | LGSNRAS | 414 | MQALQTPLT | 470 |
| 34 | NYLD | | | | | |
| BCMA- | RASQSVSSSYLA | 405 | GTSSRAT | 436 | QHYGNSPPKF | 471 |
| 35 | | | | | T | |
| BCMA- | RASQSVASSFLA | 408 | GASGRAT | 437 | QHYGGSPRLT | 472 |
| 36 | | | | | | |
| BCMA- | RASQDVNTAVS | 409 | SASYRYT | 438 | QQHYSTPWT | 473 |
| 37 | | | | | | |
| BCMA- | RASESVSVIGAH | 410 | LASNLET | 439 | LQSRIFPRT | 474 |
| 38 | LIH | | | | | |
| BCMA- | RASESVTILGSH | 411 | LASNVQT | 440 | LQSRTIPRT | 475 |
| 39 | LIY | | | | | |
| BCMA- | RASESVTILGSH | 411 | LASNVQT | 440 | LQSRTIPRT | 475 |
| 40 | LIY | | | | | |

TABLE 12C
BCMA Binders - Light chain CDR sequences
according to Chothia numbering scheme

| | | SEQ | | SEQ | | SEQ |
|---|---|---|---|---|---|---|
| Anti- | | ID | CDR- | ID | | ID |
| body | CDR-L1 | NO: | L2 | NO: | CDR-L3 | NO: |
| BCMA-1 | SQSISSY | 476 | AAS | 507 | SYSTPY | 520 |
| BCMA-2 | SQSISSSF | 477 | GAS | 508 | YHSSPSW | 521 |
| BCMA-3 | SQSLLHSNGYNY | 478 | LGS | 509 | ALQTPY | 522 |
| BCMA-4 | SQSLLRNDGKTP | 479 | EVS | 510 | NIQFP | 523 |
| BCMA-5 | SQSLLHSNGYNY | 478 | LGS | 509 | ALQTPY | 522 |
| BCMA-6 | SQSISSY | 476 | GAS | 508 | SYKRA | 524 |
| BCMA-7 | SQSLLYSNGYNY | 480 | LGS | 509 | GRQFPY | 525 |
| BCMA-8 | SQSVSSN | 481 | GAS | 508 | YGSSL | 526 |
| BCMA-9 | SQSVSSK | 482 | GAS | 508 | YGSSSW | 527 |
| BCMA-10 | SQSVGSTN | 483 | DAS | 217 | YGSSPPW | 528 |
| BCMA-11 | SQSISSY | 476 | AAS | 507 | SYTL | 529 |
| BCMA-12 | SESLVHNSGKTY | 484 | EVS | 510 | GTHWPG | 530 |
| BCMA-13 | SEDINKF | 485 | DAS | 217 | YESLPL | 531 |
| BCMA-14 | SQSVGSN | 486 | GAS | 508 | YNDWLPV | 532 |
| BCMA-15 | SQSIGSSS | 487 | GAS | 508 | YAGSPPF | 533 |
| BCMA-16 | SQDIDDA | 488 | SAT | 511 | HDNFPL | 534 |
| BCMA-17 | SQDIYNN | 489 | AAN | 512 | YYRFPY | 535 |
| BCMA-18 | SQSLLHSNGYNY | 478 | LGS | 509 | ALQTPY | 522 |
| BCMA-19 | NNIGTKS | 490 | DDS | 513 | WDSDSEHV | 536 |
| BCMA-20 | DGLSKKY | 491 | RDK | 514 | WDDTTV | 537 |
| BCMA-21 | SQGIRNW | 492 | AAS | 507 | YNSAPF | 538 |
| BCMA-22 | NNIGSKS | 493 | GKN | 515 | RDSSGDHLR | 539 |
| BCMA-23 | DSLGNYY | 494 | GTN | 213 | RDSSGHHL | 540 |
| BCMA-24 | SQSVSSAY | 495 | GAS | 508 | YGSSFNGSSLF | 541 |
| BCMA-25 | SQSVSNS | 496 | DAS | 217 | FGTSSGL | 542 |
| BCMA-26 | SQSVSSSF | 497 | GAS | 508 | YHSSPSW | 521 |
| BCMA-27 | SQSVSTTF | 498 | GSS | 516 | YHSSPSW | 521 |
| BCMA-28 | SQSISSY | 476 | AAS | 507 | SYSTPY | 520 |
| BCMA-29 | TQSIGSSF | 499 | GAS | 508 | YESSPSW | 543 |
| BCMA-30 | SQSVSSSY | 500 | GAS | 508 | YGSPPRF | 544 |
| BCMA-31 | SQSVSSSY | 500 | GAS | 508 | YGSSPSW | 545 |
| BCMA-32 | SQRVASNY | 501 | GAS | 508 | YDSSPSW | 546 |
| BCMA-33 | SQSLSSNF | 502 | GAS | 508 | YGTSPMY | 547 |
| BCMA-34 | SQSLLHSNGYNY | 478 | LGS | 509 | ALQTPL | 548 |
| BCMA-35 | SQSVSSSY | 500 | GTS | 517 | YGNSPPKF | 549 |
| BCMA-36 | SQSVASSF | 503 | GAS | 508 | YGGSPRL | 550 |
| BCMA-37 | SQDVNTA | 504 | SAS | 518 | HYSTPW | 551 |
| BCMA-38 | SESVSVIGAHL | 505 | LAS | 519 | SRIFPR | 552 |
| BCMA-39 | SESVTILGSHL | 506 | LAS | 519 | SRTIPR | 553 |
| BCMA-40 | SESVTILGSHL | 506 | LAS | 519 | SRTIPR | 553 |

TABLE 12D
BCMA Binders - Light chain CDR sequences according
to combination of Kabat and Chothia numbering schemes

| | | SEQ | | SEQ | | SEQ |
|---|---|---|---|---|---|---|
| Antibody | CDR-L1 | ID NO: | CDR-L2 | ID NO: | CDR-L3 | ID NO: |
| BCMA-1 | RASQSISSYLN | 380 | AASSLQS | 412 | QQSYSTPYT | 441 |
| BCMA-2 | RASQSISSSFLA | 381 | GASRRAT | 413 | QQYHSSPSWT | 442 |
| BCMA-3 | RSSQSLLHSNGYN | 382 | LGSNRAS | 414 | MQALQTPYT | 443 |
| | YLD | | | | | |
| BCMA-4 | KSSQSLLRNDGKT | 383 | EVSNRFS | 415 | MQNIQFPS | 444 |
| | PLY | | | | | |
| BCMA-5 | RSSQSLLHSNGYN | 384 | LGSKRAS | 416 | MQALQTPYT | 443 |
| | YLN | | | | | |
| BCMA-6 | RASQSISSYLN | 380 | GASTLAS | 417 | QQSYKRAS | 445 |
| BCMA-7 | RSSQSLLYSNGYN | 385 | LGSNRAS | 414 | MQGRQFPYS | 446 |
| | YVD | | | | | |
| BCMA-8 | RASQSVSSNLA | 386 | GASTRAS | 418 | QQYGSSLT | 447 |
| BCMA-9 | RASQSVSSKLA | 387 | GASIRAT | 419 | QQYGSSSWT | 448 |
| BCMA-10 | RASQSVGSTNLA | 388 | DASNRAT | 158 | QQYGSSPPWT | 449 |
| BCMA-11 | RASQSISSYLN | 380 | AASSLQS | 412 | QQSYTLA | 450 |
| BCMA-12 | KSSESLVHNSGKT | 389 | EVSNRDS | 420 | MQGTHWPGT | 451 |
| | YLN | | | | | |
| BCMA-13 | QASEDINKFLN | 390 | DASTLQT | 421 | QQYESLPLT | 452 |
| BCMA-14 | RASQSVGSNLA | 391 | GASTRAT | 422 | QQYNDWLPVT | 453 |
| BCMA-15 | RASQSIGSSSLA | 392 | GASSRAS | 423 | QQYAGSPPFT | 454 |
| BCMA-16 | KASQDIDDAMN | 393 | SATSPVP | 424 | LQHDNFPLT | 455 |
| BCMA-17 | RASQDIYNNLA | 394 | AANKSQS | 425 | QHYYRFPYS | 456 |
| BCMA-18 | RSSQSLLHSNGYN | 382 | LGSNRAS | 414 | MQALQTPYT | 443 |
| | YLD | | | | | |
| BCMA-19 | GGNNIGTKSVH | 395 | DDSVRPS | 426 | QVWDSDSEHVV | 457 |
| BCMA-20 | SGDGLSKKYVS | 396 | RDKERPS | 427 | QAWDDTTVV | 458 |
| BCMA-21 | RASQGIRNWLA | 397 | AASNLQS | 428 | QKYNSAPFT | 459 |
| BCMA-22 | GGNNIGSKSVH | 398 | GKNNRPS | 429 | SSRDSSGDHLRV | 460 |
| BCMA-23 | QGDSLGNYYAT | 399 | GTNNRPS | 430 | NSRDSSGHHL | 461 |
| BCMA-24 | RASQSVSSAYLA | 400 | GASTRAT | 422 | QHYGSSFNGSSL | 462 |
| | | | | | FT | |
| BCMA-25 | RASQSVSNSLA | 401 | DASSRAT | 431 | QQFGTSSGLT | 463 |
| BCMA-26 | RASQSVSSSFLA | 402 | GASSRAT | 432 | QQYHSSPSWT | 442 |
| BCMA-27 | RASQSVSTTFLA | 403 | GSSNRAT | 433 | QQYHSSPSWT | 442 |
| BCMA-28 | RASQSISSYLN | 380 | AASSLQS | 412 | QQSYSTPYS | 464 |
| BCMA-29 | RATQSIGSSFLA | 404 | GASQRAT | 434 | QHYESSPSWT | 465 |
| BCMA-30 | RASQSVSSSYLA | 405 | GASSRAT | 432 | QQYGSPPRFT | 466 |
| BCMA-31 | RASQSVSSSYLA | 405 | GASSRAT | 432 | QHYGSSPSWT | 467 |
| BCMA-32 | RASQRVASNYLA | 406 | GASSRAT | 432 | QHYDSSPSWT | 468 |
| BCMA-33 | RASQSLSSNFLA | 407 | GASNWAT | 435 | QYYGTSPMYT | 469 |
| BCMA-34 | RSSQSLLHSNGYN | 382 | LGSNRAS | 414 | MQALQTPLT | 470 |
| | YLD | | | | | |
| BCMA-35 | RASQSVSSSYLA | 405 | GTSSRAT | 436 | QHYGNSPPKFT | 471 |
| BCMA-36 | RASQSVASSFLA | 408 | GASGRAT | 437 | QHYGGSPRLT | 472 |
| BCMA-37 | RASQDVNTAVS | 409 | SASYRYT | 438 | QQHYSTPWT | 473 |
| BCMA-38 | RASESVSVIGAHLI | 410 | LASNLET | 439 | LQSRIFPRT | 474 |
| | H | | | | | |
| BCMA-39 | RASESVTILGSHLI | 411 | LASNVQT | 440 | LQSRTIPRT | 475 |
| | Y | | | | | |
| BCMA-40 | RASESVTILGSHLI | 411 | LASNVQT | 440 | LQSRTIPRT | 475 |
| | Y | | | | | |

TABLE 12E
BCMA Binders - Heavy chain CDR sequences
according to Kabat numbering scheme

| | | SEQ | | SEQ | | SEQ |
|---|---|---|---|---|---|---|
| Antibody | CDR-H1 | ID NO: | CDR-H2 | ID NO: | CDR-H3 | ID NO: |
| BCMA-1 | NHGMS | 554 | GIVYSGSTYYAASVK | 573 | HGGESDV | 595 |
| | | | G | | | |
| BCMA-2 | NYAMS | 555 | GISRSGENTYYADSV | 574 | SPAHYYGGMDV | 596 |
| | | | KG | | | |
| BCMA-3 | DYAMH | 556 | GISWNSGSIGYADSV | 575 | HSFLAY | 597 |
| | | | KG | | | |
| BCMA-4 | NHGMS | 554 | GIVYSGSTYYAASVK | 573 | HGGESDV | 595 |
| | | | G | | | |
| BCMA-5 | NFGIN | 557 | WINPKNNNTNYAQKF | 576 | GPYYYQSYMDV | 598 |
| | | | QG | | | |
| BCMA-6 | SDAMT | 558 | VISGSGGTTYYADSV | 577 | LDSSGYYYARGPR | 599 |
| | | | KG | | Y | |
| BCMA-7 | NYGIT | 559 | WISAYNGNTNYAQKF | 578 | GPYYYYMDV | 600 |
| | | | QG | | | |
| BCMA-8 | NHGMS | 554 | GIVYSGSTYYAASVK | 573 | HGGESDV | 595 |
| | | | G | | | |
| BCMA-9 | NHGMS | 554 | GIVYSGSTYYAASVK | 573 | HGGESDV | 595 |
| | | | G | | | |
| BCMA-10 | NHGMS | 554 | GIVYSGSTYYAASVK | 573 | HGGESDV | 595 |
| | | | G | | | |
| BCMA-11 | DYYMS | 560 | YISSSGSTIYYADSVK | 579 | ESGDGMDV | 601 |
| | | | G | | | |
| BCMA-12 | DYYMS | 560 | YISSSGNTIYYADSVK | 580 | STMVREDY | 602 |
| | | | G | | | |
| BCMA-13 | NHGMS | 554 | GIVYSGSTYYAASVK | 573 | HGGESDV | 595 |
| | | | G | | | |
| BCMA-14 | NHGMS | 554 | GIVYSGSTYYAASVK | 573 | HGGESDV | 595 |
| | | | G | | | |
| BCMA-15 | NHGMS | 554 | GIVYSGSTYYAASVK | 573 | HGGESDV | 595 |
| | | | G | | | |
| BCMA-16 | SSYYYW | 561 | SIYYSGSAYYNPSLKS | 581 | HWQEWPDAFDI | 603 |
| | G | | | | | |
| BCMA-17 | TSGMCV | 562 | RIDWDEDKFYSTSLK | 582 | SGAGGTSATAFDI | 604 |
| | S | | T | | | |
| BCMA-18 | SYSMN | 563 | SISSSSSYIYYADSVK | 583 | TIAAVYAFDI | 605 |
| | | | G | | | |
| BCMA-19 | DYYMS | 560 | YISSSGSTIYYADSVK | 579 | DLRGAFDI | 606 |
| | | | G | | | |
| BCMA-20 | SHYIH | 564 | MINPSGGVTAYSQTL | 584 | EGSGSGVVYFDF | 607 |
| | | | QG | | | |
| BCMA-21 | SGGYYW | 565 | YIYYSGSTYYNPSLKS | 585 | AGIAARLRGAFDI | 608 |
| | S | | | | | |
| BCMA-22 | SYAIS | 566 | GIIPIFGTANYAQKFQ | 586 | RGGYQLLRWDVG | 609 |
| | | | G | | LLRSAFDI | |
| BCMA-23 | SNSAAW | 567 | RTYYRSKVVYSFYAISL | 587 | SSPEGLFLYWFDP | 610 |
| | N | | KS | | | |
| BCMA-24 | SYAMS | 568 | AISGSGGSTYYADSV | 588 | VEGSGSLDY | 611 |
| | | | KG | | | |
| BCMA-25 | RYPMS | 569 | GISDSGVSTYYADSA | 589 | RAGSEASDI | 612 |
| | | | KG | | | |
| BCMA-26 | SYAMS | 568 | AISGSGGSTYYADSV | 588 | ATYKRELRYYYGM | 613 |
| | | | KG | | DV | |
| BCMA-27 | SYAMS | 568 | AISGSGGSTYYADSV | 588 | ATYKRELRYYYGM | 613 |
| | | | KG | | DV | |
| BCMA-28 | DYAMH | 556 | GISWNSGSIGYADSV | 575 | VGKAVPDV | 614 |
| | | | KG | | | |
| BCMA-29 | DYAMH | 556 | SINWKGNSLAYGDSV | 590 | HQGVAYYNYAMD | 615 |
| | | | KG | | V | |
| BCMA-30 | SYAMS | 568 | AISGSGGSTYYADSV | 588 | VVRDGMDV | 616 |
| | | | KG | | | |
| BCMA-31 | SYAMS | 568 | AISGSGGSTYYADSV | 588 | IPQTGTFDY | 617 |
| | | | KG | | | |
| BCMA-32 | SYAMS | 568 | AISGSGGSTYYADSV | 588 | ANYKRELRYYYGM | 618 |
| | | | KG | | DV | |
| BCMA-33 | SYAMS | 568 | AISGSGGSTYYADSV | 588 | ALVGATGAFDI | 619 |
| | | | KG | | | |
| BCMA-34 | SYAMS | 568 | AISGSGGSTYYADSV | 588 | WFGEGFDP | 620 |
| | | | KG | | | |
| BCMA-35 | SYAMS | 568 | AISGSGGSTYYADSV | 588 | VGYDSSGYYRDYY | 621 |
| | | | KG | | GMDV | |
| BCMA-36 | SYAMS | 568 | AISGSGGSTYYADSV | 588 | MGWSSGYLGAFDI | 622 |
| | | | KG | | | |
| BCMA-37 | NFGMN | 570 | WINTYTGESYFADDF | 591 | GEIYYGYDGGFAY | 623 |
| | | | KG | | | |
| BCMA-38 | DYSIN | 571 | WINTETREPAYAYDF | 592 | DYSYAMDY | 624 |
| | | | RG | | | |
| BCMA-39 | HYSMN | 572 | RINTESGVPIYADDFK | 593 | DYLYSLDF | 625 |
| | | | G | | | |
| BCMA-40 | HYSMN | 572 | RINTETGEPLYADDFK | 594 | DYLYSCDY | 626 |
| | | | G | | | |

TABLE 12F
BCMA Binders - Heavy chain CDR sequences according
to Chothia numbering scheme

| | | SEQ | | SEQ | | SEQ |
|---|---|---|---|---|---|---|
| Antibody | CDR-H1 | ID NO: | CDR-H2 | ID NO: | CDR-H3 | ID NO: |
| BCMA-1 | GFALSNH | 627 | VYSGS | 647 | HGGESDV | 595 |
| BCMA-2 | GFTFSNY | 628 | SRSGEN | 648 | SPAHYYGGMDV | 596 |
| BCMA-3 | GFTFDDY | 629 | SWNSGS | 649 | HSFLAY | 597 |
| BCMA-4 | GFALSNH | 627 | VYSGS | 647 | HGGESDV | 595 |
| BCMA-5 | GYIFDNF | 630 | NPKNNN | 650 | GPYYYQSYMDV | 598 |
| BCMA-6 | GFTFSSD | 631 | SGSGGT | 651 | LDSSGYYYARGPRY | 599 |
| BCMA-7 | GYTFSNY | 632 | SAYNGN | 652 | GPYYYYMDV | 600 |
| BCMA-8 | GFALSNH | 627 | VYSGS | 647 | HGGESDV | 595 |
| BCMA-9 | GFALSNH | 627 | VYSGS | 647 | HGGESDV | 595 |
| BCMA-10 | GFALSNH | 627 | VYSGS | 647 | HGGESDV | 595 |
| BCMA-11 | GFTFSDY | 633 | SSSGST | 653 | ESGDGMDV | 601 |
| BCMA-12 | GFTFSDY | 633 | SSSGNT | 654 | STMVREDY | 602 |
| BCMA-13 | GFALSNH | 627 | VYSGS | 647 | HGGESDV | 595 |
| BCMA-14 | GFALSNH | 627 | VYSGS | 647 | HGGESDV | 595 |
| BCMA-15 | GFALSNH | 627 | VYSGS | 647 | HGGESDV | 595 |
| BCMA-16 | GGSISSS | 634 | YYSGS | 655 | HWQEWPDAFDI | 603 |
| | YY | | | | | |
| BCMA-17 | GFSLRTS | 635 | DWDED | 656 | SGAGGTSATAFDI | 604 |
| | GM | | | | | |
| BCMA-18 | GFTFSSY | 636 | SSSSSY | 657 | TIAAVYAFDI | 605 |
| BCMA-19 | GFTFSDY | 633 | SSSGST | 653 | DLRGAFDI | 606 |
| BCMA-20 | GYTVTSH | 637 | NPSGGV | 658 | EGSGSGWYFDF | 607 |
| BCMA-21 | GGSISSG | 638 | YYSGS | 655 | AGIAARLRGAFDI | 608 |
| | GY | | | | | |
| BCMA-22 | GGTFSSY | 639 | IPIFGT | 659 | RGGYQLLRWDVGLLRS | 609 |
| | | | | | AFDI | |
| BCMA-23 | GDSVSSN | 640 | YYRSKWY | 660 | SSPEGLFLYWFDP | 610 |
| | SA | | | | | |
| BCMA-24 | GFTFSSY | 636 | SGSGGS | 661 | VEGSGSLDY | 611 |
| BCMA-25 | GITFSRY | 641 | SDSGVS | 662 | RAGSEASDI | 612 |
| BCMA-26 | GFTFSSY | 636 | SGSGGS | 661 | ATYKRELRYYYGMDV | 613 |
| BCMA-27 | GFTFSSY | 636 | SGSGGS | 661 | ATYKRELRYYYGMDV | 613 |
| BCMA-28 | GFTFDDY | 629 | SWNSGS | 649 | VGKAVPDV | 614 |
| BCMA-29 | GFTFDDY | 629 | NWKGNS | 663 | HQGVAYYNYAMDV | 615 |
| BCMA-30 | GFTFSSY | 636 | SGSGGS | 661 | VVRDGMDV | 616 |
| BCMA-31 | GFTFSSY | 636 | SGSGGS | 661 | IPQTGTFDY | 617 |
| BCMA-32 | GFTFSSY | 636 | SGSGGS | 661 | ANYKRELRYYYGMDV | 618 |
| BCMA-33 | GFSFSSY | 642 | SGSGGS | 661 | ALVGATGAFDI | 619 |
| BCMA-34 | GFTFSSY | 636 | SGSGGS | 661 | WFGEGFDP | 620 |
| BCMA-35 | GFTFSSY | 636 | SGSGGS | 661 | VGYDSSGYYRDYYGMD | 621 |
| | | | | | V | |
| BCMA-36 | GFTFSSY | 636 | SGSGGS | 661 | MGWSSGYLGAFDI | 622 |
| BCMA-37 | GYTFTNF | 643 | NTYTGE | 664 | GEIYYGYDGGFAY | 623 |
| BCMA-38 | GYTFTDY | 644 | NTETRE | 665 | DYSYAMDY | 624 |
| BCMA-39 | GYTFRHY | 645 | NTESGV | 666 | DYLYSLDF | 625 |
| BCMA-40 | GYTFTHY | 646 | NTETGE | 667 | DYLYSCDY | 626 |

TABLE 12G
BCMA Binders - Heavy chain CDR sequences according to
combination of Kabat and Chothia numbering schemes

| | | SEQ | | SEQ | | SEQ |
|---|---|---|---|---|---|---|
| Antibody | CDR-H1 | ID NO: | CDR-H2 | ID NO: | CDR-H3 | ID NO: |
| BCMA-1 | GFALSNHG | 668 | GIVYSGSTYYAAS | 573 | HGGESDV | 595 |
| | MS | | VKG | | | |
| BCMA-2 | GFTFSNYA | 669 | GISRSGENTYYAD | 574 | SPAHYYGGMDV | 596 |
| | MS | | SVKG | | | |
| BCMA-3 | GFTFDDYA | 670 | GISWNSGSIGYAD | 575 | HSFLAY | 597 |
| | MH | | SVKG | | | |
| BCMA-4 | GFALSNHG | 668 | GIVYSGSTYYAAS | 573 | HGGESDV | 595 |
| | MS | | VKG | | | |
| BCMA-5 | GYIFDNFGI | 671 | WINPKNNNTNYA | 576 | GPYYYQSYMDV | 598 |
| | N | | QKFQG | | | |
| BCMA-6 | GFTFSSDA | 672 | VISGSGGTTYYAD | 577 | LDSSGYYYARGPR | 599 |
| | MT | | SVKG | | Y | |
| BCMA-7 | GYTFSNYG | 673 | WISAYNGNTNYA | 578 | GPYYYYMDV | 600 |
| | IT | | QKFQG | | | |
| BCMA-8 | GFALSNHG | 668 | GIVYSGSTYYAAS | 573 | HGGESDV | 595 |
| | MS | | VKG | | | |
| BCMA-9 | GFALSNHG | 668 | GIVYSGSTYYAAS | 573 | HGGESDV | 595 |
| | MS | | VKG | | | |
| BCMA-10 | GFALSNHG | 668 | GIVYSGSTYYAAS | 573 | HGGESDV | 595 |
| | MS | | VKG | | | |
| BCMA-11 | GFTFSDYY | 674 | YISSSGSTIYYADS | 579 | ESGDGMDV | 601 |
| | MS | | VKG | | | |
| BCMA-12 | GFTFSDYY | 674 | YISSSGNTIYYAD | 580 | STMVREDY | 602 |
| | MS | | SVKG | | | |
| BCMA-13 | GFALSNHG | 668 | GIVYSGSTYYAAS | 573 | HGGESDV | 595 |
| | MS | | VKG | | | |
| BCMA-14 | GFALSNHG | 668 | GIVYSGSTYYAAS | 573 | HGGESDV | 595 |
| | MS | | VKG | | | |
| BCMA-15 | GFALSNHG | 668 | GIVYSGSTYYAAS | 573 | HGGESDV | 595 |
| | MS | | VKG | | | |
| BCMA-16 | GGSISSSY | 675 | SIYYSGSAYYNPS | 581 | HWQEWPDAFDI | 603 |
| | YYWG | | LKS | | | |
| BCMA-17 | GFSLRTSG | 676 | RIDWDEDKFYSTS | 582 | SGAGGTSATAFDI | 604 |
| | MCVS | | LKT | | | |
| BCMA-18 | GFTFSSYS | 677 | SISSSSSYIYYADS | 583 | TIAAVYAFDI | 605 |
| | MN | | VKG | | | |
| BCMA-19 | GFTFSDYY | 674 | YISSSGSTIYYADS | 579 | DLRGAFDI | 606 |
| | MS | | VKG | | | |
| BCMA-20 | GYTVTSHYI | 678 | MINPSGGVTAYS | 584 | EGSGSGWYFDF | 607 |
| | H | | QTLQG | | | |
| BCMA-21 | GGSISSGG | 679 | YIYYSGSTYYNPS | 585 | AGIAARLRGAFDI | 608 |
| | YYWS | | LKS | | | |
| BCMA-22 | GGTFSSYAI | 680 | GIIPIFGTANYAQK | 586 | RGGYQLLRWDVGL | 609 |
| | S | | FQG | | LRSAFDI | |
| BCMA-23 | GDSVSSNS | 681 | RTYYRSKWYSFY | 587 | SSPEGLFLYWFDP | 610 |
| | AAWN | | AISLKS | | | |
| BCMA-24 | GFTFSSYA | 682 | AISGSGGSTYYAD | 588 | VEGSGSLDY | 611 |
| | MS | | SVKG | | | |
| BCMA-25 | GITFSRYP | 683 | GISDSGVSTYYAD | 589 | RAGSEASDI | 612 |
| | MS | | SAKG | | | |
| BCMA-26 | GFTFSSYA | 682 | AISGSGGSTYYAD | 588 | ATYKRELRYYYGM | 613 |
| | MS | | SVKG | | DV | |
| BCMA-27 | GFTFSSYA | 682 | AISGSGGSTYYAD | 588 | ATYKRELRYYYGM | 613 |
| | MS | | SVKG | | DV | |
| BCMA-28 | GFTFDDYA | 670 | GISWNSGSIGYAD | 575 | VGKAVPDV | 614 |
| | MH | | SVKG | | | |
| BCMA-29 | GFTFDDYA | 670 | SINWKGNSLAYG | 590 | HQGVAYYNYAMDV | 615 |
| | MH | | DSVKG | | | |
| BCMA-30 | GFTFSSYA | 682 | AISGSGGSTYYAD | 588 | VVRDGMDV | 616 |
| | MS | | SVKG | | | |
| BCMA-31 | GFTFSSYA | 682 | AISGSGGSTYYAD | 588 | IPQTGTFDY | 617 |
| | MS | | SVKG | | | |
| BCMA-32 | GFTFSSYA | 682 | AISGSGGSTYYAD | 588 | ANYKRELRYYYGM | 618 |
| | MS | | SVKG | | DV | |
| BCMA-33 | GFSFSSYA | 684 | AISGSGGSTYYAD | 588 | ALVGATGAFDI | 619 |
| | MS | | SVKG | | | |
| BCMA-34 | GFTFSSYA | 682 | AISGSGGSTYYAD | 588 | WFGEGFDP | 620 |
| | MS | | SVKG | | | |
| BCMA-35 | GFTFSSYA | 682 | AISGSGGSTYYAD | 588 | VGYDSSGYYRDYY | 621 |
| | MS | | SVKG | | GMDV | |
| BCMA-36 | GFTFSSYA | 682 | AISGSGGSTYYAD | 588 | MGWSSGYLGAFDI | 622 |
| | MS | | SVKG | | | |
| BCMA-37 | GYTFTNFG | 685 | WINTYTGESYFAD | 591 | GEIYYGYDGGFAY | 623 |
| | MN | | DFKG | | | |
| BCMA-38 | GYTFTDYSI | 686 | WINTETREPAYAY | 592 | DYSYAMDY | 624 |
| | N | | DFRG | | | |
| BCMA-39 | GYTFRHYS | 687 | RINTESGVPIYAD | 593 | DYLYSLDF | 625 |
| | MN | | DFKG | | | |
| BCMA-40 | GYTFTHYS | 688 | RINTETGEPLYAD | 594 | DYLYSCDY | 626 |
| | MN | | DFKG | | | |

In some embodiments, the ABM comprises the CDR sequences of BCMA-1. In some embodiments, the ABM comprises the CDR sequences of BCMA-2. In some embodiments, the ABM comprises the CDR sequences of BCMA-3. In some embodiments, the ABM comprises the CDR sequences of BCMA-4. In some embodiments, the ABM comprises the CDR sequences of BCMA-5. In some embodiments, the ABM comprises the CDR sequences of BCMA-6. In some embodiments, the ABM comprises the CDR sequences of BCMA-7. In some embodiments, the ABM comprises the CDR sequences of BCMA-8. In some embodiments, the ABM comprises the CDR sequences of BCMA-9. In some embodiments, the ABM comprises the CDR sequences of BCMA-10. In some embodiments, the ABM comprises the CDR sequences of BCMA-11. In some embodiments, the ABM comprises the CDR sequences of BCMA-12. In some embodiments, the ABM comprises the CDR sequences of BCMA-13. In some embodiments, the ABM comprises the CDR sequences of BCMA-14. In some embodiments, the ABM comprises the CDR sequences of BCMA-15. In some embodiments, the ABM comprises the CDR sequences of BCMA-16. In some embodiments, the ABM comprises the CDR sequences of BCMA-17. In some embodiments, the ABM comprises the CDR sequences of BCMA-18. In some embodiments, the ABM comprises the CDR sequences of BCMA-19. In some embodiments, the ABM comprises the CDR sequences of BCMA-20. In some embodiments, the ABM comprises the CDR sequences of BCMA-21. In some embodiments, the ABM comprises the CDR sequences of BCMA-22. In some embodiments, the ABM comprises the CDR sequences of BCMA-23. In some embodiments, the ABM comprises the CDR sequences of BCMA-24. In some embodiments, the ABM comprises the CDR sequences of BCMA-25. In some embodiments, the ABM comprises the CDR sequences of BCMA-26. In some embodiments, the ABM comprises the CDR sequences of BCMA-27. In some embodiments, the ABM comprises the CDR sequences of BCMA-28. In some embodiments, the ABM comprises the CDR sequences of BCMA-29. In some embodiments, the ABM comprises the CDR sequences of BCMA-30. In some embodiments, the ABM comprises the CDR sequences of BCMA-31. In some embodiments, the ABM comprises the CDR sequences of BCMA-32. In some embodiments, the ABM comprises the CDR sequences of BCMA-33. In some embodiments, the ABM comprises the CDR sequences of BCMA-34. In some embodiments, the ABM comprises the CDR sequences of BCMA-35. In some embodiments, the ABM comprises the CDR sequences of BCMA-36. In some embodiments, the ABM comprises the CDR sequences of BCMA-37. In some embodiments, the ABM comprises the CDR sequences of BCMA-38. In some embodiments, the ABM comprises the CDR sequences of BCMA-39. In some embodiments, the ABM comprises the CDR sequences of BCMA-40.

In some embodiments, the CDRs are defined by Kabat numbering, as set forth in Table 12B and 10E. In other embodiments, the CDRs are defined by Chothia numbering, as set forth in Table 12C and 10F. In yet other embodiments, the CDRs are defined by a combination of Kabat and Chothia numbering, as set forth in Table 12D and 10G.

In some embodiments, the TBMs comprising a ABM that binds to BCMA can comprise the heavy and light chain variable sequences of any of BCMA-1 to BCMA-40.

In some embodiments, the ABM comprises the heavy and light chain variable sequences of BCMA-1, as set forth in Table 12A. In some embodiments, the ABM comprises the heavy and light chain variable sequences of BCMA-2, as set forth in Table 12A. In some embodiments, the ABM comprises the heavy and light chain variable sequences of BCMA-3, as set forth in Table 12A. In some embodiments, the ABM comprises the heavy and light chain variable sequences of BCMA-4, as set forth in Table 12A. In some embodiments, the ABM comprises the heavy and light chain variable sequences of BCMA-5, as set forth in Table 12A. In some embodiments, the ABM comprises the heavy and light chain variable sequences of BCMA-6, as set forth in Table 12A. In some embodiments, the ABM comprises the heavy and light chain variable sequences of BCMA-7, as set forth in Table 12A. In some embodiments, the ABM comprises the heavy and light chain variable sequences of BCMA-8, as set forth in Table 12A. In some embodiments, the ABM comprises the heavy and light chain variable sequences of BCMA-9, as set forth in Table 12A. In some embodiments, the ABM comprises the heavy and light chain variable sequences of BCMA-10, as set forth in Table 12A. In some embodiments, the ABM comprises the heavy and light chain variable sequences of BCMA-11, as set forth in Table 12A. In some embodiments, the ABM comprises the heavy and light chain variable sequences of BCMA-12, as set forth in Table 12A. In some embodiments, the ABM comprises the heavy and light chain variable sequences of BCMA-13, as set forth in Table 12A. In some embodiments, the ABM comprises the heavy and light chain variable sequences of BCMA-14, as set forth in Table 12A. In some embodiments, the ABM comprises the heavy and light chain variable sequences of BCMA-15, as set forth in Table 12A. In some embodiments, the ABM comprises the heavy and light chain variable sequences of BCMA-16, as set forth in Table 12A. In some embodiments, the ABM comprises the heavy and light chain variable sequences of BCMA-17, as set forth in Table 12A. In some embodiments, the ABM comprises the heavy and light chain variable sequences of BCMA-18, as set forth in Table 12A. In some embodiments, the ABM comprises the heavy and light chain variable sequences of BCMA-19, as set forth in Table 12A. In some embodiments, the ABM comprises the heavy and light chain variable sequences of BCMA-20, as set forth in Table 12A. In some embodiments, the ABM comprises the heavy and light chain variable sequences of BCMA-21, as set forth in Table 12A. In some embodiments, the ABM comprises the heavy and light chain variable sequences of BCMA-22, as set forth in Table 12A.

In some embodiments, the ABM comprises the heavy and light chain variable sequences of BCMA-23, as set forth in Table 12A. In some embodiments, the ABM comprises the heavy and light chain variable sequences of BCMA-24, as set forth in Table 12A. In some embodiments, the ABM comprises the heavy and light chain variable sequences of BCMA-25, as set forth in Table 12A. In some embodiments, the ABM comprises the heavy and light chain variable sequences of BCMA-26, as set forth in Table 12A. In some embodiments, the ABM comprises the heavy and light chain variable sequences of BCMA-27, as set forth in Table 12A. In some embodiments, the ABM comprises the heavy and light chain variable sequences of BCMA-28, as set forth in Table 12A. In some embodiments, the ABM comprises the heavy and light chain variable sequences of BCMA-29, as set forth in Table 12A. In some embodiments, the ABM comprises the heavy and light chain variable sequences of BCMA-30, as set forth in Table 12A. In some embodiments, the ABM comprises the heavy and light chain variable sequences of BCMA-31, as set forth in Table 12A. In some embodiments, the ABM comprises the heavy and light chain variable sequences of BCMA-32, as set forth in Table 12A. In some embodiments, the ABM comprises the heavy and light chain variable sequences of BCMA-33, as set forth in Table 12A. In some embodiments, the ABM comprises the heavy and light chain variable sequences of BCMA-34, as set forth in Table 12A. In some embodiments, the ABM comprises the heavy and light chain variable sequences of BCMA-35, as set forth in Table 12A. In some embodiments, the ABM comprises the heavy and light chain variable sequences of BCMA-36, as set forth in Table 12A. In some embodiments, the ABM comprises the heavy and light chain variable sequences of BCMA-37, as set forth in Table 12A. In some embodiments, the ABM comprises the heavy and light chain variable sequences of BCMA-38, as set forth in Table 12A. In some embodiments, the ABM comprises the heavy and light chain variable sequences of BCMA-39, as set forth in Table 12A. In some embodiments, the ABM comprises the heavy and light chain variable sequences of BCMA-40, as set forth in Table 12A.

6.7.2. CD19

B cells express cell surface proteins which can be utilized as markers for differentiation and identification. One such human B-cell marker is a CD19 antigen and is found on mature B cells but not on plasma cells. CD19 is expressed during early pre-B cell development and remains until plasma cell differentiation. CD19 is expressed on both normal B cells and malignant B cells whose abnormal growth can lead to B-cell lymphomas. For example, CD19 is expressed on B-cell lineage malignancies, including, but not limited to non-Hodgkin's lymphoma (B-NHL), chronic lymphocytic leukemia, and acute lymphoblastic leukemia.

In certain aspects, a TBM of the disclosure comprises an ABM3 that specifically binds to CD19. Exemplary CDR and variable domain sequences that can be incorporated into an ABM3 that specifically binds to CD19 are set forth in Table 13 below

TABLE 13
CD19 Binders

| | | | SEQ |
|---|---|---|---|
| | | | ID |
| Name | Domain | Sequence | NO: |
| CD19-H1 | CDR-H1 | DYGVS | 689 |
| CD19-H2A | CDR-H2 | VIWGSETTYYNSALKS | 690 |
| CD19-H2B | CDR-H2 | VIWGSETTYYSSSLKS | 691 |
| CD19-H2C | CDR-H2 | VIWGSETTYYQSSLKS | 692 |
| CD19-H2D | CDR-H2 | VIWGSETTYYNSSLKS | 693 |
| CD19-H3 | CDR-H3 | HYYYGGSYAMDY | 694 |
| CD19-L1 | CDR-L1 | RASQDISKYLN | 695 |
| CD19-L2 | CDR-L2 | HTSRLHS | 696 |
| CD19-L3 | CDR-L3 | QQGNTLPYT | 697 |
| CD19-VHA | VH | EVKLQESGPGLVAPSQSLSVTCTVSGVSLPDYGVSWI | 698 |
| | | RQPPRKGLEWLGVIWGSETTYYNSALKSRLTIIKDNSK | |
| | | SQVFLKMNSLQTDDTAIYYCAKHYYYGGSYAMDYWG | |
| | | QGTSVTVSS | |
| CD19-VHB | VH | QVQLQESGPGLVKPSETLSLTCTVSGVSLPDYGVSWIR | 699 |
| | | QPPGKGLEWIGVIWGSETTYYSSSLKSRVTISKDNSKN | |
| | | QVSLKLSSVTAADTAVYYCAKHYYYGGSYAMDYWGQ | |
| | | GTLVTVSS | |
| CD19-VHC | VH | QVQLQESGPGLVKPSETLSLTCTVSGVSLPDYGVSWIR | 700 |
| | | QPPGKGLEWIGVIWGSETTYYQSSLKSRVTISKDNSKN | |
| | | QVSLKLSSVTAADTAVYYCAKHYYYGGSYAMDYWGQ | |
| | | GTLVTVSS | |
| CD19-VHD | VH | QVQLQESGPGLVKPSETLSLTCTVSGVSLPDYGVSWIR | 701 |
| | | QPPGKGLEWIGVIWGSETTYYNSSLKSRVTISKDNSKN | |
| | | QVSLKLSSVTAADTAVYYCAKHYYYGGSYAMDYWGQ | |
| | | GTLVTVSS | |
| CD19-VLA | VL | DIQMTQTTSSLSASLGDRVTISCRASQDISKYLNWYQQ | 702 |
| | | KPDGTVKLLIYHTSRLHSGVPSRFSGSGSGTDYSLTISN | |
| | | LEQEDIATYFCQQGNTLPYTFGGGTKLEIT | |
| CD19-VLB | VL | EIVMTQSPATLSLSPGERATLSCRASQDISKYLNWYQQ | 703 |
| | | KPGQAPRLLIYHTSRLHSGIPARFSGSGSGTDYTLTISS | |
| | | LQPEDFAVYFCQQGNTLPYTFGQGTKLEIK | |
| CD19-scFv1 | scFv | EIVMTQSPATLSLSPGERATLSCRASQDISKYLNWYQQ | 704 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVSL | |
| | | PDYGVSWIRQPPGKGLEWIGVIWGSETTYYSSSLKSR | |
| | | VTISKDNSKNQVSLKLSSVTAADTAVYYCAKHYYYGGS | |
| | | YAMDYWGQGTLVTVSS | |
| CD19-scFv2 | scFv | EIVMTQSPATLSLSPGERATLSCRASQDISKYLNWYQQ | 705 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVSL | |
| | | PDYGVSWIRQPPGKGLEWIGVIWGSETTYYQSSLKSR | |
| | | VTISKDNSKNQVSLKLSSVTAADTAVYYCAKHYYYGGS | |
| | | YAMDYWGQGTLVTVSS | |
| CD19-scFv3 | scFv | QVQLQESGPGLVKPSETLSLTCTVSGVSLPDYGVSWIR | 706 |
| | | QPPGKGLEWIGVIWGSETTYYSSSLKSRVTISKDNSKN | |
| | | QVSLKLSSVTAADTAVYYCAKHYYYGGSYAMDYWGQ | |
| | | GTLVTVSSGGGGSGGGGSGGGGSEIVMTQSPATLSLS | |
| | | PGERATLSCRASQDISKYLNWYQQKPGQAPRLLIYHTS | |
| | | RLHSGIPARFSGSGSGTDYTLTISSLQPEDFAVYFCQQ | |
| | | GNTLPYTFGQGTKLEIK | |
| CD19-scFv4 | scFv | QVQLQESGPGLVKPSETLSLTCTVSGVSLPDYGVSWIR | 707 |
| | | QPPGKGLEWIGVIWGSETTYYQSSLKSRVTISKDNSKN | |
| | | QVSLKLSSVTAADTAVYYCAKHYYYGGSYAMDYWGQ | |
| | | GTLVTVSSGGGGSGGGGSGGGGSEIVMTQSPATLSLS | |
| | | PGERATLSCRASQDISKYLNWYQQKPGQAPRLLIYHTS | |
| | | RLHSGIPARFSGSGSGTDYTLTISSLQPEDFAVYFCQQ | |
| | | GNTLPYTFGQGTKLEIK | |
| CD19-scFv5 | scFv | EIVMTQSPATLSLSPGERATLSCRASQDISKYLNWYQQ | 708 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYS | |
| | | SSLKSRVTISKDNSKNQVSLKLSSVTAADTAVYYCAKH | |
| | | YYYGGSYAMDYWGQGTLVTVSS | |
| CD19-scFv6 | scFv | EIVMTQSPATLSLSPGERATLSCRASQDISKYLNWYQQ | 709 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYQ | |
| | | SSLKSRVTISKDNSKNQVSLKLSSVTAADTAVYYCAKH | |
| | | YYYGGSYAMDYWGQGTLVTVSS | |
| CD19-scFv7 | scFv | QVQLQESGPGLVKPSETLSLTCTVSGVSLPDYGVSWIR | 710 |
| | | QPPGKGLEWIGVIWGSETTYYSSSLKSRVTISKDNSKN | |
| | | QVSLKLSSVTAADTAVYYCAKHYYYGGSYAMDYWGQ | |
| | | GTLVTVSSGGGGSGGGGSGGGGSGGGGSEIVMTQS | |
| | | PATLSLSPGERATLSCRASQDISKYLNVVYQQKPGQAP | |
| | | RLLIYHTSRLHSGIPARFSGSGSGTDYTLTISSLQPEDF | |
| | | AVYFCQQGNTLPYTFGQGTKLEIK | |
| CD19-scFv8 | scFv | QVQLQESGPGLVKPSETLSLTCTVSGVSLPDYGVSWIR | 711 |
| | | QPPGKGLEWIGVIWGSETTYYQSSLKSRVTISKDNSKN | |
| | | QVSLKLSSVTAADTAVYYCAKHYYYGGSYAMDYWGQ | |
| | | GTLVTVSSGGGGSGGGGSGGGGSGGGGSEIVMTQS | |
| | | PATLSLSPGERATLSCRASQDISKYLNWYQQKPGQAP | |
| | | RLLIYHTSRLHSGIPARFSGSGSGTDYTLTISSLQPEDF | |
| | | AVYFCQQGNTLPYTFGQGTKLEIK | |
| CD19-scFv9 | scFv | EIVMTQSPATLSLSPGERATLSCRASQDISKYLNWYQQ | 712 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYN | |
| | | SSLKSRVTISKDNSKNQVSLKLSSVTAADTAVYYCAKH | |
| | | YYYGGSYAMDYWGQGTLVTVSS | |
| CD19- | scFv | QVQLQESGPGLVKPSETLSLTCTVSGVSLPDYGVSWIR | 713 |
| scFv10 | | QPPGKGLEWIGVIWGSETTYYNSSLKSRVTISKDNSKN | |
| | | QVSLKLSSVTAADTAVYYCAKHYYYGGSYAMDYWGQ | |
| | | GTLVTVSSGGGGSGGGGSGGGGSGGGGSEIVMTQS | |
| | | PATLSLSPGERATLSCRASQDISKYLNWYQQKPGQAP | |
| | | RLLIYHTSRLHSGIPARFSGSGSGTDYTLTISSLQPEDF | |
| | | AVYFCQQGNTLPYTFGQGTKLEIK | |
| CD19- | scFv | EIVMTQSPATLSLSPGERATLSCRASQDISKYLNWYQQ | 714 |
| scFv11 | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVSL | |
| | | PDYGVSWIRQPPGKGLEWIGVIWGSETTYYNSSLKSR | |
| | | VTISKDNSKNQVSLKLSSVTAADTAVYYCAKHYYYGGS | |
| | | YAMDYWGQGTLVTVSS | |
| CD19- | scFv | QVQLQESGPGLVKPSETLSLTCTVSGVSLPDYGVSWIR | 715 |
| scFv12 | | QPPGKGLEWIGVIWGSETTYYNSSLKSRVTISKDNSKN | |
| | | QVSLKLSSVTAADTAVYYCAKHYYYGGSYAMDYWGQ | |
| | | GTLVTVSSGGGGSGGGGSGGGGSEIVMTQSPATLSLS | |
| | | PGERATLSCRASQDISKYLNWYQQKPGQAPRLLIYHTS | |
| | | RLHSGIPARFSGSGSGTDYTLTISSLQPEDFAVYFCQQ | |
| | | GNTLPYTFGQGTKLEIK | |

In certain aspects, the ABM3 comprises heavy chain CDRs having the amino acid sequences of CD19-H1, CD19-H2A, and CD19-H3 as set forth in Table 13 and light chain CDRs having the amino acid sequences of CD19-L1, CD19-L2, and CD19-L3 as set forth in Table 13. In a specific embodiment, the ABM3 comprises a heavy chain variable region having the amino acid sequences of VHA as set forth in Table 13 and a light chain variable region having the amino acid sequences of VLA as set forth in Table 13.

In other aspects, the ABM3 comprises heavy chain CDRs having the amino acid sequences of CD19-H1, CD19-H2B, and CD19-H3 as set forth in Table 13 and light chain CDRs having the amino acid sequences of CD19-L1, CD19-L2, and CD19-L3 as set forth in Table 13. In a specific embodiment, the ABM3 comprises a heavy chain variable region having the amino acid sequences of VHB as set forth in Table 13 and a light chain variable region having the amino acid sequences of VLB as set forth in Table 13.

In further aspects, the ABM3 comprises heavy chain CDRs having the amino acid sequences of CD19-H1, CD19-H2C, and CD19-H3 as set forth in Table 13 and light chain CDRs having the amino acid sequences of CD19-L1, CD19-L2, and CD19-L3 as set forth in Table 13. In a specific embodiment, ABM3 comprises a heavy chain variable region having the amino acid sequences of VHC as set forth in Table 13 and a light chain variable region having the amino acid sequences of VLB as set forth in Table 13.

In further aspects, the ABM3 comprises heavy chain CDRs having the amino acid sequences of CD19-H1, CD19-H2D, and CD19-H3 as set forth in Table 13 and light chain CDRs having the amino acid sequences of CD19-L1, CD19-L2, and CD19-L3 as set forth in Table 13. In a specific embodiment, the ABM3 comprises a heavy chain variable region having the amino acid sequences of VHD as set forth in Table 13 and a light chain variable region having the amino acid sequences of VLB as set forth in Table 13.

In yet further aspects, the ABM3 is in the form of an scFV. Exemplary anti-CD19 scFvs comprise the amino acid sequence of any one of CD19-scFv1 through CD19-scFv12 as set forth in Table 13.

### 6.8. Nucleic Acids and Host Cells

In another aspect, the disclosure provides nucleic acids encoding the TBMs of the disclosure. In some embodiments, the TBMs are encoded by a single nucleic acid. In other embodiments, the TBMs are encoded by a plurality (e.g., two, three, four or more) nucleic acids.

A single nucleic acid can encode a TBM that comprises a single polypeptide chain, a TBM that comprises two or more polypeptide chains, or a portion of a TBM that comprises more than two polypeptide chains (for example, a single nucleic acid can encode two polypeptide chains of a TBM comprising three, four or more polypeptide chains, or three polypeptide chains of a TBM comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a TBM comprising two or more polypeptide chains is encoded by two or more nucleic acids. The number of nucleic acids encoding a TBM can be equal to or less than the number of polypeptide chains in the TBM (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

6.8.1. Vectors

The disclosure provides vectors comprising nucleotide sequences encoding a TBM or a TBM component described herein. In one embodiment, the vectors comprise nucleotides encoding an immunoglobulin-based ABM described herein. In one embodiment, the vectors comprise nucleotides encoding an Fc domain described herein. In one embodiment, the vectors comprise nucleotides encoding a recombinant non-immunoglobulin based ABM described herein. A vector of the disclosure can encode one or more ABMs, one or more Fc domains, one or more non-immunoglobulin based ABM, or a combination thereof (e.g., when multiple components or sub-components are encoded as a single polypeptide chain). In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

6.8.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.9. Antibody-Drug Conjugates

The TBMs of the disclosure can be conjugated, e.g., via a linker, to a drug moiety. Such conjugates are referred to herein as antibody-drug conjugates (or “ADCs”) for convenience, notwithstanding the fact that one or more (or all) of the ABMs might be based on non-immunoglobulin scaffolds.

In certain aspects, the drug moiety exerts a cytotoxic or cytostatic activity. In one embodiment, the drug moiety is chosen from a maytansinoid, a kinesin-like protein KIF11 inhibitor, a V-ATPase (vacuolar-type H+-ATPase) inhibitor, a pro-apoptotic agent, a Bcl2 (B-cell lymphoma 2) inhibitor, an MCL1 (myeloid cell leukemia 1) inhibitor, a HSP90 (heat shock protein 90) inhibitor, an IAP (inhibitor of apoptosis) inhibitor, an mTOR (mechanistic target of rapamycin) inhibitor, a microtubule stabilizer, a microtubule destabilizer, an auristatin, a dolastatin, a MetAP (methionine aminopeptidase), a CRM1 (chromosomal maintenance 1) inhibitor, a DPPIV (dipeptidyl peptidase IV) inhibitor, a proteasome inhibitor, an inhibitor of a phosphoryl transfer reaction in mitochondria, a protein synthesis inhibitor, a kinase inhibitor, a CDK2 (cyclin-dependent kinase 2) inhibitor, a CDK9 (cyclin-dependent kinase 9) inhibitor, a kinesin inhibitor, an HDAC (histone deacetylase) inhibitor, a DNA damaging agent, a DNA alkylating agent, a DNA intercalator, a DNA minor groove binder, a RNA polymerase inhibitor, a topoisomerase inhibitor, or a DHFR (dihydrofolate reductase) inhibitor.

In one embodiment, the linker is chosen from a cleavable linker, a non-cleavable linker, a hydrophilic linker, a procharged linker, or a dicarboxylic acid based linker.

In specific embodiments, the ADCs are compounds according to structural formula (I):

[D-L-XY]ₙ-Ab

or salts thereof, where each “D” represents, independently of the others, a cytotoxic and/or cytostatic agent (“drug”); each “L” represents, independently of the others, a linker; “Ab” represents a TBM described herein; each “XY” represents a linkage formed between a functional group R^{x} on the linker and a “complementary” functional group R^{y} on the antibody, and n represents the number of drugs linked to, or drug-to-antibody ratio (DAR), of the ADC.

Specific embodiments of the various antibodies (Ab) that can comprise the ADCs include the various embodiments of TBMs described above.

In some specific embodiments of the ADCs and/or salts of structural formula (I), each D is the same and/or each L is the same.

Specific embodiments of cytotoxic and/or cytostatic agents (D) and linkers (L) that can comprise the ADCs of the disclosure, as well as the number of cytotoxic and/or cytostatic agents linked to the ADCs, are described in more detail below.

6.9.1. Cytotoxic and/or Cytostatic Agents

The cytotoxic and/or cytostatic agents may be any agents known to inhibit the growth and/or replication of and/or kill cells, and in particular cancer and/or tumor cells. Numerous agents having cytotoxic and/or cytostatic properties are known in the literature. Non-limiting examples of classes of cytotoxic and/or cytostatic agents include, by way of example and not limitation, radionuclides, alkylating agents, topoisomerase I inhibitors, topoisomerase II inhibitors, DNA intercalating agents (e.g., groove binding agents such as minor groove binders), RNA/DNA antimetabolites, cell cycle modulators, kinase inhibitors, protein synthesis inhibitors, histone deacetylase inhibitors, mitochondria inhibitors, and antimitotic agents.

Specific non-limiting examples of agents within certain of these various classes are provided below.

Alkylating Agents: asaley ((L-Leucine, N—[N-acetyl-4-[bis-(2-chloroethyl)amino]-DL-phenylalanyl]-, ethylester; NSC 167780; CAS Registry No. 3577897)); AZQ ((1,4-cyclohexadiene-1,4-dicarbamic acid, 2,5-bis(1-aziridinyl)-3,6-dioxo-, diethyl ester; NSC 182986; CAS Registry No. 57998682)); BCNU ((N,N′-Bis(2-chloroethyl)-N-nitrosourea; NSC 409962; CAS Registry No. 154938)); busulfan (1,4-butanediol dimethanesulfonate; NSC 750; CAS Registry No. 55981); (carboxyphthalato)platinum (NSC 27164; CAS Registry No. 65296813); CBDCA ((cis-(1,1-cyclobutanedicarboxylato)diammineplatinum(II)); NSC 241240; CAS Registry No. 41575944)); CCNU ((N-(2-chloroethyl)-N′-cyclohexyl-N-nitrosourea; NSC 79037; CAS Registry No. 13010474)); CHIP (iproplatin; NSC 256927); chlorambucil (NSC 3088; CAS Registry No. 305033); chlorozotocin ((2-[[[(2-chloroethyl) nitrosoamino]carbonyl]amino]-2-deoxy-D-glucopyranose; NSC 178248; CAS Registry No. 54749905)); cis-platinum (cisplatin; NSC 119875; CAS Registry No. 15663271); clomesone (NSC 338947; CAS Registry No. 88343720); cyanomorpholinodoxorubicin (NCS 357704; CAS Registry No. 88254073); cyclodisone (NSC 348948; CAS Registry No. 99591738); dianhydrogalactitol (5,6-diepoxydulcitol; NSC 132313; CAS Registry No. 23261203); fluorodopan ((5-[(2-chloroethyl)-(2-fluoroethyl)amino]-6-methyl-uracil; NSC 73754; CAS Registry No. 834913); hepsulfam (NSC 329680; CAS Registry No. 96892578); hycanthone (NSC 142982; CAS Registry No. 23255938); melphalan (NSC 8806; CAS Registry No. 3223072); methyl CCNU ((1-(2-chloroethyl)-3-(trans-4-methylcyclohexane)-1-nitrosourea; NSC 95441; 13909096); mitomycin C (NSC 26980; CAS Registry No. 50077); mitozolamide (NSC 353451; CAS Registry No. 85622953); nitrogen mustard ((bis(2-chloroethyl)methylamine hydrochloride; NSC 762; CAS Registry No. 55867); PCNU ((1-(2-chloroethyl)-3-(2,6-dioxo-3-piperidyl)-1-nitrosourea; NSC 95466; CAS Registry No. 13909029)); piperazine alkylator ((1-(2-chloroethyl)-4-(3-chloropropyl)-piperazine dihydrochloride; NSC 344007)); piperazinedione (NSC 135758; CAS Registry No. 41109802); pipobroman ((N,N-bis(3-bromopropionyl) piperazine; NSC 25154; CAS Registry No. 54911)); porfiromycin (N-methylmitomycin C; NSC 56410; CAS Registry No. 801525); spirohydantoin mustard (NSC 172112; CAS Registry No. 56605164); teroxirone (triglycidylisocyanurate; NSC 296934; CAS Registry No. 2451629); tetraplatin (NSC 363812; CAS Registry No. 62816982); thio-tepa (N,N′,N″-tri-1,2-ethanediylthio phosphoramide; NSC 6396; CAS Registry No. 52244); triethylenemelamine (NSC 9706; CAS Registry No. 51183); uracil nitrogen mustard (desmethyldopan; NSC 34462; CAS Registry No. 66751); Yoshi-864 ((bis(3-mesyloxy propyl)amine hydrochloride; NSC 102627; CAS Registry No. 3458228).

Topoisomerase I Inhibitors: camptothecin (NSC 94600; CAS Registry No. 7689-03-4); various camptothecin derivatives and analogs (for example, NSC 100880, NSC 603071, NSC 107124, NSC 643833, NSC 629971, NSC 295500, NSC 249910, NSC 606985, NSC 74028, NSC 176323, NSC 295501, NSC 606172, NSC 606173, NSC 610458, NSC 618939, NSC 610457, NSC 610459, NSC 606499, NSC 610456, NSC 364830, and NSC 606497); morpholinisoxorubicin (NSC 354646; CAS Registry No. 89196043); SN-38 (NSC 673596; CAS Registry No. 86639-52-3).

Topoisomerase II Inhibitors: doxorubicin (NSC 123127; CAS Registry No. 25316409); amonafide (benzisoquinolinedione; NSC 308847; CAS Registry No. 69408817); m-AMSA ((4′-(9-acridinylamino)-3′-methoxymethanesulfonanilide; NSC 249992; CAS Registry No. 51264143)); anthrapyrazole derivative ((NSC 355644); etoposide (VP-16; NSC 141540; CAS Registry No. 33419420); pyrazoloacridine ((pyrazolo[3,4,5-kl]acridine-2(6H)-propanamine, 9-methoxy-N, N-dimethyl-5-nitro-, monomethanesulfonate; NSC 366140; CAS Registry No. 99009219); bisantrene hydrochloride (NSC 337766; CAS Registry No. 71439684); daunorubicin (NSC 821151; CAS Registry No. 23541506); deoxydoxorubicin (NSC 267469; CAS Registry No. 63950061); mitoxantrone (NSC 301739; CAS Registry No. 70476823); menogaril (NSC 269148; CAS Registry No. 71628961); N,N-dibenzyl daunomycin (NSC 268242; CAS Registry No. 70878512); oxanthrazole (NSC 349174; CAS Registry No. 105118125); rubidazone (NSC 164011; CAS Registry No. 36508711); teniposide (VM-26; NSC 122819; CAS Registry No. 29767202).

DNA Intercalating Agents: anthramycin (CAS Registry No. 4803274); chicamycin A (CAS Registry No. 89675376); tomaymycin (CAS Registry No. 35050556); DC-81 (CAS Registry No. 81307246); sibiromycin (CAS Registry No. 12684332); pyrrolobenzodiazepine derivative (CAS Registry No. 945490095); SGD-1882 ((S)-2-(4-aminophenyl)-7-methoxy-8-(3-4(S)-7-methoxy-2-(4-methoxyphenyl)-5-oxo-5,11a-dihydro-1H-benzo[e]pyrrolo[1,2-a][1,4]diazepin-8-yl)oxy)propox-y)-1H-benzo[e]pyrrolo[1,2-a][1,4]diazepin-5(11aH)-one); SG2000 (SJG-136; (11aS,11a′S)-8,8′-(propane-1,3-diylbis(oxy))bis(7-methoxy-2-methylene-2,3-dihydro-1H-benzo[e]pyrrolo[1,2-a][1,4]diazepin-5(11aH)-one); NSC 694501; CAS Registry No. 232931576).

RNA/DNA Antimetabolites: L-alanosine (NSC 153353; CAS Registry No. 59163416); 5-azacytidine (NSC 102816; CAS Registry No. 320672); 5-fluorouracil (NSC 19893; CAS Registry No. 51218); acivicin (NSC 163501; CAS Registry No. 42228922); aminopterin derivative N-[2-chloro-5-[[(2,4-diamino-5-methyl-6-quinazolinyl)methyl]amino]benzoyl-]L-aspartic acid (NSC 132483); aminopterin derivative N44-[[(2,4-diamino-5-ethyl-6-quinazolinyl)methyl]amino]benzoyl]L-asparti-c acid (NSC 184692); aminopterin derivative N-[2-chloro-4-[[(2,4-diamino-6-pteridinyl)methyl]amino]benzoyl]L-aspartic acid monohydrate (NSC 134033); an antifo ((N^{α}-(4-amino-4-deoxypteroyl)-N⁷-hemiphthaloyl-L-ornithin-e; NSC 623017)); Baker's soluble antifol (NSC 139105; CAS Registry No. 41191042); dichlorallyl lawsone ((2-(3,3-dichloroallyl)-3-hydroxy-1,4-naphthoquinone; NSC 126771; CAS Registry No. 36417160); brequinar (NSC 368390; CAS Registry No. 96201886); ftorafur ((pro-drug; 5-fluoro-1-(tetrahydro-2-furyl)-uracil; NSC 148958; CAS Registry No. 37076689); 5,6-dihydro-5-azacytidine (NSC 264880; CAS Registry No. 62402317); methotrexate (NSC 740; CAS Registry No. 59052); methotrexate derivative (N-[[4-[[(2,4-diamino-6-pteridinyl)methyl]methylamino]-1-naphthalenyl]car-bonyl]L-glutamic acid; NSC 174121); PALA ((N-(phosphonoacetyl)-L-aspartate; NSC 224131; CAS Registry No. 603425565); pyrazofurin (NSC 143095; CAS Registry No. 30868305); trimetrexate (NSC 352122; CAS Registry No. 82952645).

DNA Antimetabolites: 3-HP (NSC 95678; CAS Registry No. 3814797); 2′-deoxy-5-fluorouridine (NSC 27640; CAS Registry No. 50919); 5-HP (NSC 107392; CAS Registry No. 19494894); α-TGDR (α-2′-deoxy-6-thioguanosine; NSC 71851 CAS Registry No. 2133815); aphidicolin glycinate (NSC 303812; CAS Registry No. 92802822); ara C (cytosine arabinoside; NSC 63878; CAS Registry No. 69749); 5-aza-2′-deoxycytidine (NSC 127716; CAS Registry No. 2353335); β-TGDR (β-2′-deoxy-6-thioguanosine; NSC 71261; CAS Registry No. 789617); cyclocytidine (NSC 145668; CAS Registry No. 10212256); guanazole (NSC 1895; CAS Registry No. 1455772); hydroxyurea (NSC 32065; CAS Registry No. 127071); inosine glycodialdehyde (NSC 118994; CAS Registry No. 23590990); macbecin II (NSC 330500; CAS Registry No. 73341738); pyrazoloimidazole (NSC 51143; CAS Registry No. 6714290); thioguanine (NSC 752; CAS Registry No. 154427); thiopurine (NSC 755; CAS Registry No. 50442).

Cell Cycle Modulators: silibinin (CAS Registry No. 22888-70-6); epigallocatechin gallate (EGCG; CAS Registry No. 989515); procyanidin derivatives (e.g., procyanidin A1 [CAS Registry No. 103883030], procyanidin B1 [CAS Registry No. 20315257], procyanidin B4 [CAS Registry No. 29106512], arecatannin B1 [CAS Registry No. 79763283]); isoflavones (e.g., genistein [4′,5,7-trihydroxyisoflavone; CAS Registry No. 446720], daidzein [4′,7-dihydroxyisoflavone, CAS Registry No. 486668]; indole-3-carbinol (CAS Registry No. 700061); quercetin (NSC 9219; CAS Registry No. 117395); estramustine (NSC 89201; CAS Registry No. 2998574); nocodazole (CAS Registry No. 31430189); podophyllotoxin (CAS Registry No. 518285); vinorelbine tartrate (NSC 608210; CAS Registry No. 125317397); cryptophycin (NSC 667642; CAS Registry No. 124689652).

Kinase Inhibitors: afatinib (CAS Registry No. 850140726); axitinib (CAS Registry No. 319460850); ARRY-438162 (binimetinib) (CAS Registry No. 606143899); bosutinib (CAS Registry No. 380843754); cabozantinib (CAS Registry No. 1140909483); ceritinib (CAS Registry No. 1032900256); crizotinib (CAS Registry No. 877399525); dabrafenib (CAS Registry No. 1195765457); dasatinib (NSC 732517; CAS Registry No. 302962498); erlotinib (NSC 718781; CAS Registry No. 183319699); everolimus (NSC 733504; CAS Registry No. 159351696); fostamatinib (NSC 745942; CAS Registry No. 901119355); gefitinib (NSC 715055; CAS Registry No. 184475352); ibrutinib (CAS Registry No. 936563961); imatinib (NSC 716051; CAS Registry No. 220127571); lapatinib (CAS Registry No. 388082788); lenvatinib (CAS Registry No. 857890392); mubritinib (CAS 366017096); nilotinib (CAS Registry No. 923288953); nintedanib (CAS Registry No. 656247175); palbociclib (CAS Registry No. 571190302); pazopanib (NSC 737754; CAS Registry No. 635702646); pegaptanib (CAS Registry No. 222716861); ponatinib (CAS Registry No. 1114544318); rapamycin (NSC 226080; CAS Registry No. 53123889); regorafenib (CAS Registry No. 755037037); AP 23573 (ridaforolimus) (CAS Registry No. 572924540); INCB018424 (ruxolitinib) (CAS Registry No. 1092939177); ARRY-142886 (selumetinib) (NSC 741078; CAS Registry No. 606143-52-6); sirolimus (NSC 226080; CAS Registry No. 53123889); sorafenib (NSC 724772; CAS Registry No. 475207591); sunitinib (NSC 736511; CAS Registry No. 341031547); tofacitinib (CAS Registry No. 477600752); temsirolimus (NSC 683864; CAS Registry No. 163635043); trametinib (CAS Registry No. 871700173); vandetanib (CAS Registry No. 443913733); vemurafenib (CAS Registry No. 918504651); SU6656 (CAS Registry No. 330161870); CEP-701 (lesaurtinib) (CAS Registry No. 111358884); XL019 (CAS Registry No. 945755566); PD-325901 (CAS Registry No. 391210109); PD-98059 (CAS Registry No. 167869218); ATP-competitive TORC1/TORC2 inhibitors including PI-103 (CAS Registry No. 371935749), PP242 (CAS Registry No. 1092351671), PP30 (CAS Registry No. 1092788094), Torin 1 (CAS Registry No. 1222998368), LY294002 (CAS Registry No. 154447366), XL-147 (CAS Registry No. 934526893), CAL-120 (CAS Registry No. 870281348), ETP-45658 (CAS Registry No. 1198357797), PX 866 (CAS Registry No. 502632668), GDC-0941 (CAS Registry No. 957054307), BGT226 (CAS Registry No. 1245537681), BEZ235 (CAS Registry No. 915019657), XL-765 (CAS Registry No. 934493762).

Protein Synthesis Inhibitors: acriflavine (CAS Registry No. 65589700); amikacin (NSC 177001; CAS Registry No. 39831555); arbekacin (CAS Registry No. 51025855); astromicin (CAS Registry No. 55779061); azithromycin (NSC 643732; CAS Registry No. 83905015); bekanamycin (CAS Registry No. 4696768); chlortetracycline (NSC 13252; CAS Registry No. 64722); clarithromycin (NSC 643733; CAS Registry No. 81103119); clindamycin (CAS Registry No. 18323449); clomocycline (CAS Registry No. 1181540); cycloheximide (CAS Registry No. 66819); dactinomycin (NSC 3053; CAS Registry No. 50760); dalfopristin (CAS Registry No. 112362502); demeclocycline (CAS Registry No. 127333); dibekacin (CAS Registry No. 34493986); dihydrostreptomycin (CAS Registry No. 128461); dirithromycin (CAS Registry No. 62013041); doxycycline (CAS Registry No. 17086281); emetine (NSC 33669; CAS Registry No. 483181); erythromycin (NSC 55929; CAS Registry No. 114078); flurithromycin (CAS Registry No. 83664208); framycetin (neomycin B; CAS Registry No. 119040); gentamycin (NSC 82261; CAS Registry No. 1403663); glycylcyclines, such as tigecycline (CAS Registry No. 220620097); hygromycin B (CAS Registry No. 31282049); isepamicin (CAS Registry No. 67814760); josamycin (NSC 122223; CAS Registry No. 16846245); kanamycin (CAS Registry No. 8063078); ketolides such as telithromycin (CAS Registry No. 191114484), cethromycin (CAS Registry No. 205110481), and solithromycin (CAS Registry No. 760981837); lincomycin (CAS Registry No. 154212); lymecycline (CAS Registry No. 992212); meclocycline (NSC 78502; CAS Registry No. 2013583); metacycline (rondomycin; NSC 356463; CAS Registry No. 914001); midecamycin (CAS Registry No. 35457808); minocycline (NSC 141993; CAS Registry No. 10118908); miocamycin (CAS Registry No. 55881077); neomycin (CAS Registry No. 119040); netilmicin (CAS Registry No. 56391561); oleandomycin (CAS Registry No. 3922905); oxazolidinones, such as eperezolid (CAS Registry No. 165800044), linezolid (CAS Registry No. 165800033), posizolid (CAS Registry No. 252260029), radezolid (CAS Registry No. 869884786), ranbezolid (CAS Registry No. 392659380), sutezolid (CAS Registry No. 168828588), tedizolid (CAS Registry No. 856867555); oxytetracycline (NSC 9169; CAS Registry No. 2058460); paromomycin (CAS Registry No. 7542372); penimepicycline (CAS Registry No. 4599604); peptidyl transferase inhibitors, e.g., chloramphenicol (NSC 3069; CAS Registry No. 56757) and derivatives such as azidamfenicol (CAS Registry No. 13838089), florfenicol (CAS Registry No. 73231342), and thiamphenicol (CAS Registry No. 15318453), and pleuromutilins such as retapamulin (CAS Registry No. 224452668), tiamulin (CAS Registry No. 55297955), valnemulin (CAS Registry No. 101312929); pirlimycin (CAS Registry No. 79548735); puromycin (NSC 3055; CAS Registry No. 53792); quinupristin (CAS Registry No. 120138503); ribostamycin (CAS Registry No. 53797356); rokitamycin (CAS Registry No. 74014510); rolitetracycline (CAS Registry No. 751973); roxithromycin (CAS Registry No. 80214831); sisomicin (CAS Registry No. 32385118); spectinomycin (CAS Registry No. 1695778); spiramycin (CAS Registry No. 8025818); streptogramins such as pristinamycin (CAS Registry No. 270076603), quinupristin/dalfopristin (CAS Registry No. 126602899), and virginiamycin (CAS Registry No. 11006761); streptomycin (CAS Registry No. 57921); tetracycline (NSC 108579; CAS Registry No. 60548); tobramycin (CAS Registry No. 32986564); troleandomycin (CAS Registry No. 2751099); tylosin (CAS Registry No. 1401690); verdamicin (CAS Registry No. 49863481).

Histone Deacetylase Inhibitors: abexinostat (CAS Registry No. 783355602); belinostat (NSC 726630; CAS Registry No. 414864009); chidamide (CAS Registry No. 743420022); entinostat (CAS Registry No. 209783802); givinostat (CAS Registry No. 732302997); mocetinostat (CAS Registry No. 726169739); panobinostat (CAS Registry No. 404950807); quisinostat (CAS Registry No. 875320299); resminostat (CAS Registry No. 864814880); romidepsin (CAS Registry No. 128517077); sulforaphane (CAS Registry No. 4478937); thioureidobutyronitrile (Kevetrin™; CAS Registry No. 6659890); valproic acid (NSC 93819; CAS Registry No. 99661); vorinostat (NSC 701852; CAS Registry No. 149647789); ACY-1215 (rocilinostat; CAS Registry No. 1316214524); CUDC-101 (CAS Registry No. 1012054599); CHR-2845 (tefinostat; CAS Registry No. 914382608); CHR-3996 (CAS Registry No. 1235859138); 4SC-202 (CAS Registry No. 910462430); CG200745 (CAS Registry No. 936221339); SB939 (pracinostat; CAS Registry No. 929016966).

Mitochondria Inhibitors: pancratistatin (NSC 349156; CAS Registry No. 96281311); rhodamine-123 (CAS Registry No. 63669709); edelfosine (NSC 324368; CAS Registry No. 70641519); d-alpha-tocopherol succinate (NSC 173849; CAS Registry No. 4345033); compound 11β (CAS Registry No. 865070377); aspirin (NSC 406186; CAS Registry No. 50782); ellipticine (CAS Registry No. 519233); berberine (CAS Registry No. 633658); cerulenin (CAS Registry No. 17397896); GX015-070 (Obatoclax®; 1H-Indole, 2-(2-((3,5-dimethyl-1H-pyrrol-2-yl)methylene)-3-methoxy-2H-pyrrol-5-yl)-; NSC 729280; CAS Registry No. 803712676); celastrol (tripterine; CAS Registry No. 34157830); metformin (NSC 91485; CAS Registry No. 1115704); Brilliant green (NSC 5011; CAS Registry No. 633034); ME-344 (CAS Registry No. 1374524556).

Antimitotic Agents: allocolchicine (NSC 406042); auristatins, such as MMAE (monomethyl auristatin E; CAS Registry No. 474645-27-7) and MMAF (monomethyl auristatin F; CAS Registry No. 745017-94-1; halichondrin B (NSC 609395); colchicine (NSC 757; CAS Registry No. 64868); cholchicine derivative (N-benzoyl-deacetyl benzamide; NSC 33410; CAS Registry No. 63989753); dolastatin 10 (NSC 376128; CAS Registry No 110417-88-4); maytansine (NSC 153858; CAS Registry No. 35846-53-8); rhozoxin (NSC 332598; CAS Registry No. 90996546); taxol (NSC 125973; CAS Registry No. 33069624); taxol derivative ((2′-N-[3-(dimethylamino)propyl]glutaramate taxol; NSC 608832); thiocolchicine (3-demethylthiocolchicine; NSC 361792); trityl cysteine (NSC 49842; CAS Registry No. 2799077); vinblastine sulfate (NSC 49842; CAS Registry No. 143679); vincristine sulfate (NSC 67574; CAS Registry No. 2068782).

Any of these agents that include or that may be modified to include a site of attachment to a TBM may be included in the ADCs disclosed herein.

In a specific embodiment, the cytotoxic and/or cytostatic agent is an antimitotic agent.

In another specific embodiment, the cytotoxic and/or cytostatic agent is an auristatin, for example, monomethyl auristatin E (“MMAE:) or monomethyl auristatin F (“MMAF”).

6.9.2. ADC Linkers

In the ADCs of the disclosure, the cytotoxic and/or cytostatic agents are linked to the TBM by way of ADC linkers. The ADC linker linking a cytotoxic and/or cytostatic agent to the TBM of an ADC may be short, long, hydrophobic, hydrophilic, flexible or rigid, or may be composed of segments that each independently have one or more of the above-mentioned properties such that the linker may include segments having different properties. The linkers may be polyvalent such that they covalently link more than one agent to a single site on the TBM, or monovalent such that covalently they link a single agent to a single site on the TBM.

As will be appreciated by skilled artisans, the ADC linkers link cytotoxic and/or cytostatic agents to the TBM by forming a covalent linkage to the cytotoxic and/or cytostatic agent at one location and a covalent linkage to the TBM at another. The covalent linkages are formed by reaction between functional groups on the ADC linker and functional groups on the agents and TBM. As used herein, the expression “ADC linker” is intended to include (i) unconjugated forms of the ADC linker that include a functional group capable of covalently linking the ADC linker to a cytotoxic and/or cytostatic agent and a functional group capable of covalently linking the ADC linker to a TBM; (ii) partially conjugated forms of the ADC linker that include a functional group capable of covalently linking the ADC linker to a TBM and that is covalently linked to a cytotoxic and/or cytostatic agent, or vice versa; and (iii) fully conjugated forms of the ADC linker that are covalently linked to both a cytotoxic and/or cytostatic agent and a TBM. In some specific embodiments of ADC linkers and ADCs of the disclosure, as well as synthons used to conjugate linker-agents to TBMs, moieties comprising the functional groups on the ADC linker and covalent linkages formed between the ADC linker and TBM are specifically illustrated as R, and XY, respectively.

The ADC linkers are preferably, but need not be, chemically stable to conditions outside the cell, and may be designed to cleave, immolate and/or otherwise specifically degrade inside the cell. Alternatively, ADC linkers that are not designed to specifically cleave or degrade inside the cell may be used. Choice of stable versus unstable ADC linker may depend upon the toxicity of the cytotoxic and/or cytostatic agent. For agents that are toxic to normal cells, stable linkers are preferred. Agents that are selective or targeted and have lower toxicity to normal cells may utilize, chemical stability of the ADC linker to the extracellular milieu is less important. A wide variety of ADC linkers useful for linking drugs to TBMs in the context of ADCs are known in the art. Any of these ADC linkers, as well as other ADC linkers, may be used to link the cytotoxic and/or cytostatic agents to the TBM of the ADCs of the disclosure.

Exemplary polyvalent ADC linkers that may be used to link many cytotoxic and/or cytostatic agents to a single TBM molecule are described, for example, in WO 2009/073445; WO 2010/068795; WO 2010/138719; WO 2011/120053; WO 2011/171020; WO 2013/096901; WO 2014/008375; WO 2014/093379; WO 2014/093394; WO 2014/093640, the content of which are incorporated herein by reference in their entireties. For example, the Fleximer linker technology developed by Mersana et al. has the potential to enable high-DAR ADCs with good physicochemical properties. As shown below, the Mersana technology is based on incorporating drug molecules into a solubilizing poly-acetal backbone via a sequence of ester bonds. The methodology renders highly-loaded ADCs (DAR up to 20) while maintaining good physicochemical properties.

Additional examples of dendritic type linkers can be found in US 2006/116422; US 2005/271615; de Groot et al., 2003, Angew. Chem. Int. Ed. 42:4490-4494; Amir et al., 2003, Angew. Chem. Int. Ed. 42:4494-4499; Shamis et al., 2004, J. Am. Chem. Soc. 126:1726-1731; Sun et al., 2002, Bioorganic & Medicinal Chemistry Letters 12:2213-2215; Sun et al., 2003, Bioorganic & Medicinal Chemistry 11:1761-1768; King et al., 2002, Tetrahedron Letters 43:1987-1990, each of which is incorporated herein by reference.

Exemplary monovalent ADC linkers that may be used are described, for example, in Nolting, 2013, Antibody-Drug Conjugates, Methods in Molecular Biology 1045:71-100; Kitson et al., 2013, CROs-MOs—Chemica-ggi—Chemistry Today 31(4):30-38; Ducry et al., 2010, Bioconjugate Chem. 21:5-13; Zhao et al., 2011, J. Med. Chem. 54:3606-3623; U.S. Pat. Nos. 7,223,837; 8,568,728; 8,535,678; and WO2004010957, each of which is incorporated herein by reference.

By way of example and not limitation, some cleavable and noncleavable ADC linkers that may be included in the ADCs of the disclosure are described below.

6.9.2.1. Cleavable ADC Linkers

In certain embodiments, the ADC linker selected is cleavable in vivo. Cleavable ADC linkers may include chemically or enzymatically unstable or degradable linkages. Cleavable ADC linkers generally rely on processes inside the cell to liberate the drug, such as reduction in the cytoplasm, exposure to acidic conditions in the lysosome, or cleavage by specific proteases or other enzymes within the cell. Cleavable ADC linkers generally incorporate one or more chemical bonds that are either chemically or enzymatically cleavable while the remainder of the ADC linker is noncleavable. In certain embodiments, an ADC linker comprises a chemically labile group such as hydrazone and/or disulfide groups. Linkers comprising chemically labile groups exploit differential properties between the plasma and some cytoplasmic compartments. The intracellular conditions to facilitate drug release for hydrazone containing ADC linkers are the acidic environment of endosomes and lysosomes, while the disulfide containing ADC linkers are reduced in the cytosol, which contains high thiol concentrations, e.g., glutathione. In certain embodiments, the plasma stability of an ADC linker comprising a chemically labile group may be increased by introducing steric hindrance using substituents near the chemically labile group.

Acid-labile groups, such as hydrazone, remain intact during systemic circulation in the blood's neutral pH environment (pH 7.3-7.5) and undergo hydrolysis and release the drug once the ADC is internalized into mildly acidic endosomal (pH 5.0-6.5) and lysosomal (pH 4.5-5.0) compartments of the cell. This pH dependent release mechanism has been associated with nonspecific release of the drug. To increase the stability of the hydrazone group of the ADC linker, the ADC linker may be varied by chemical modification, e.g., substitution, allowing tuning to achieve more efficient release in the lysosome with a minimized loss in circulation.

Hydrazone-containing ADC linkers may contain additional cleavage sites, such as additional acid-labile cleavage sites and/or enzymatically labile cleavage sites. ADCs including exemplary hydrazone-containing ADC linkers include the following structures:

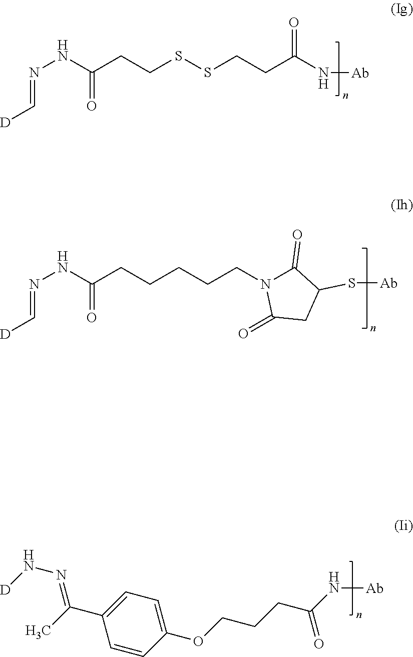

wherein D and Ab represent the cytotoxic and/or cytostatic agent (drug) and Ab, respectively, and n represents the number of drug-ADC linkers linked to the TBM. In certain ADC linkers such as linker (Ig), the ADC linker comprises two cleavable groups—a disulfide and a hydrazone moiety. For such ADC linkers, effective release of the unmodified free drug requires acidic pH or disulfide reduction and acidic pH. Linkers such as (Ih) and (Ii) have been shown to be effective with a single hydrazone cleavage site.

Additional ADC linkers which remain intact during systemic circulation and undergo hydrolysis and release the drug when the ADC is internalized into acidic cellular compartments include carbonates. Such ADC linkers can be useful in cases where the cytotoxic and/or cytostatic agent can be covalently attached through an oxygen.

Other acid-labile groups that may be included in ADC linkers include cis-aconityl-containing ADC linkers. cis-Aconityl chemistry uses a carboxylic acid juxtaposed to an amide bond to accelerate amide hydrolysis under acidic conditions.

Cleavable ADC linkers may also include a disulfide group. Disulfides are thermodynamically stable at physiological pH and are designed to release the drug upon internalization inside cells, wherein the cytosol provides a significantly more reducing environment compared to the extracellular environment. Scission of disulfide bonds generally requires the presence of a cytoplasmic thiol cofactor, such as (reduced) glutathione (GSH), such that disulfide-containing ADC linkers are reasonably stable in circulation, selectively releasing the drug in the cytosol. The intracellular enzyme protein disulfide isomerase, or similar enzymes capable of cleaving disulfide bonds, may also contribute to the preferential cleavage of disulfide bonds inside cells. GSH is reported to be present in cells in the concentration range of 0.5-10 mM compared with a significantly lower concentration of GSH or cysteine, the most abundant low-molecular weight thiol, in circulation at approximately 5 Tumor cells, where irregular blood flow leads to a hypoxic state, result in enhanced activity of reductive enzymes and therefore even higher glutathione concentrations. In certain embodiments, the in vivo stability of a disulfide-containing ADC linker may be enhanced by chemical modification of the ADC linker, e.g., use of steric hindrance adjacent to the disulfide bond.

ADCs including exemplary disulfide-containing ADC linkers include the following structures:

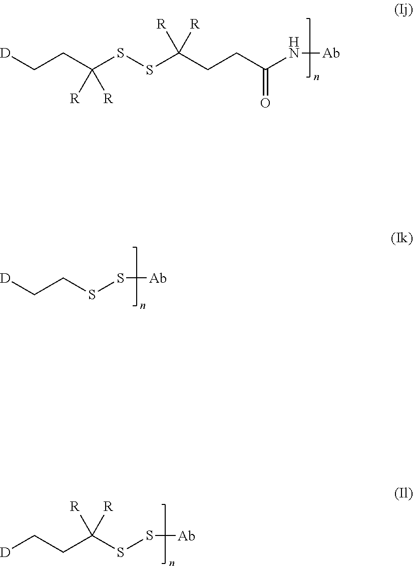

wherein D and Ab represent the drug and TBM, respectively, n represents the number of drug-ADC linkers linked to the TBM and R is independently selected at each occurrence from hydrogen or alkyl, for example. In certain embodiments, increasing steric hindrance adjacent to the disulfide bond increases the stability of the ADC linker. Structures such as (ID and (II) show increased in vivo stability when one or more R groups is selected from a lower alkyl such as methyl.

Another type of cleavable ADC linker that may be used is an ADC linker that is specifically cleaved by an enzyme. Such ADC linkers are typically peptide-based or include peptidic regions that act as substrates for enzymes. Peptide based ADC linkers tend to be more stable in plasma and extracellular milieu than chemically labile ADC linkers. Peptide bonds generally have good serum stability, as lysosomal proteolytic enzymes have very low activity in blood due to endogenous inhibitors and the unfavorably high pH value of blood compared to lysosomes. Release of a drug from a TBM occurs specifically due to the action of lysosomal proteases, e.g., cathepsin and plasmin. These proteases may be present at elevated levels in certain tumor cells.

In exemplary embodiments, the cleavable peptide is selected from tetrapeptides such as Gly-Phe-Leu-Gly (SEQ ID NO: 716), Ala-Leu-Ala-Leu (SEQ ID NO: 717) or dipeptides such as Val-Cit, Val-Ala, Met-(D)Lys, Asn-(D)Lys, Val-(D)Asp, Phe-Lys, Ile-Val, Asp-Val, His-Val, NorVal-(D)Asp, Ala-(D)Asp 5, Met-Lys, Asn-Lys, Ile-Pro, Me3Lys-Pro, PhenylGly-(D)Lys, Met-(D)Lys, Asn-(D)Lys, Pro-(D)Lys, Met-(D)Lys, Asn-(D)Lys, AM Met-(D)Lys, Asn-(D)Lys, AW Met-(D)Lys, and Asn-(D)Lys. In certain embodiments, dipeptides are preferred over longer polypeptides due to hydrophobicity of the longer peptides.

A variety of dipeptide-based cleavable ADC linkers useful for linking drugs such as doxorubicin, mitomycin, camptothecin, pyrrolobenzodiazepine, tallysomycin and auristatin/auristatin family members to TBMs have been described (see, Dubowchik et al., 1998, J. Org. Chem. 67:1866-1872; Dubowchik et al., 1998, Bioorg. Med. Chem. Lett. 8(21):3341-3346; Walker et al., 2002, Bioorg. Med. Chem. Lett. 12:217-219; Walker et al., 2004, Bioorg. Med. Chem. Lett. 14:4323-4327; Sutherland et al., 2013, Blood 122: 1455-1463; and Francisco et al., 2003, Blood 102:1458-1465, of each of which is incorporated herein by reference). All of these dipeptide ADC linkers, or modified versions of these dipeptide ADC linkers, may be used in the ADCs of the disclosure. Other dipeptide ADC linkers that may be used include those found in ADCs such as Seattle Genetics' Brentuximab Vendotin SGN-35 (Adcetris™), Seattle Genetics SGN-75 (anti-CD-70, Val-Cit-monomethyl auristatin F(MMAF), Seattle Genetics SGN-CD33A (anti-CD-33, Val-Ala-(SGD-1882)), Celldex Therapeutics glembatumumab (CDX-011) (anti-NMB, Val-Cit-monomethyl auristatin E (MMAE), and Cytogen PSMA-ADC (PSMA-ADC-1301) (anti-PSMA, Val-Cit-MMAE).

Enzymatically cleavable ADC linkers may include a self-immolative spacer to spatially separate the drug from the site of enzymatic cleavage. The direct attachment of a drug to a peptide ADC linker can result in proteolytic release of an amino acid adduct of the drug, thereby impairing its activity. The use of a self-immolative spacer allows for the elimination of the fully active, chemically unmodified drug upon amide bond hydrolysis.

One self-immolative spacer is the bifunctional para-aminobenzyl alcohol group, which is linked to the peptide through the amino group, forming an amide bond, while amine containing drugs may be attached through carbamate functionalities to the benzylic hydroxyl group of the ADC linker (PABC). The resulting prodrugs are activated upon protease-mediated cleavage, leading to a 1,6-elimination reaction releasing the unmodified drug, carbon dioxide, and remnants of the ADC linker group. The following scheme depicts the fragmentation of p-amidobenzyl ether and release of the drug:

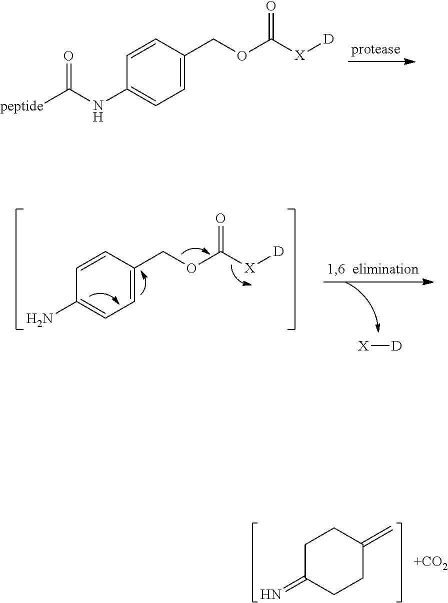

wherein X-D represents the unmodified drug.

Heterocyclic variants of this self-immolative group have also been described. See for example, U.S. Pat. No. 7,989,434, incorporated herein by reference.

In some embodiments, the enzymatically cleavable ADC linker is a β-glucuronic acid-based ADC linker. Facile release of the drug may be realized through cleavage of the β-glucuronide glycosidic bond by the lysosomal enzyme β-glucuronidase. This enzyme is present abundantly within lysosomes and is overexpressed in some tumor types, while the enzyme activity outside cells is low. β-Glucuronic acid-based ADC linkers may be used to circumvent the tendency of an ADC to undergo aggregation due to the hydrophilic nature of β-glucuronides. In some embodiments, β-glucuronic acid-based ADC linkers are preferred as ADC linkers for ADCs linked to hydrophobic drugs. The following scheme depicts the release of the drug from and ADC containing a β-glucuronic acid-based ADC linker:

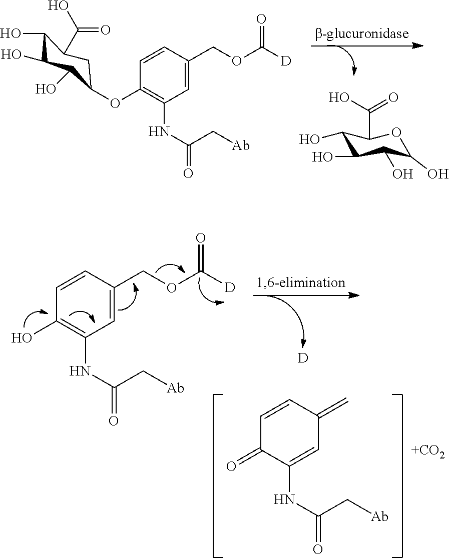

A variety of cleavable β-glucuronic acid-based ADC linkers useful for linking drugs such as auristatins, camptothecin and doxorubicin analogues, CBI minor-groove binders, and psymberin to TBMs have been described (see, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013; Jeffrey et al., 2006, Bioconjug. Chem. 17:831-840; Jeffrey et al., 2007, Bioorg. Med. Chem. Lett. 17:2278-2280; and Jiang et al., 2005, J. Am. Chem. Soc. 127:11254-11255, each of which is incorporated herein by reference). All of these β-glucuronic acid-based ADC linkers may be used in the ADCs of the disclosure.

Additionally, cytotoxic and/or cytostatic agents containing a phenol group can be covalently bonded to an ADC linker through the phenolic oxygen. One such ADC linker, described in WO 2007/089149, relies on a methodology in which a diamino-ethane “SpaceLink” is used in conjunction with traditional “PABO”-based self-immolative groups to deliver phenols. The cleavage of the ADC linker is depicted schematically below, where D represents a cytotoxic and/or cytostatic agent having a phenolic hydroxyl group.

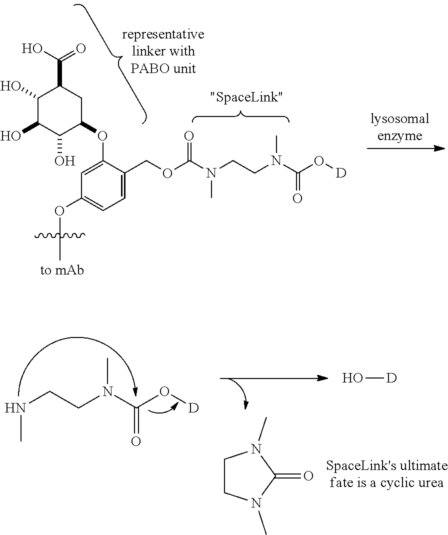

Cleavable ADC linkers may include noncleavable portions or segments, and/or cleavable segments or portions may be included in an otherwise non-cleavable ADC linker to render it cleavable. By way of example only, polyethylene glycol (PEG) and related polymers may include cleavable groups in the polymer backbone. For example, a polyethylene glycol or polymer ADC linker may include one or more cleavable groups such as a disulfide, a hydrazone or a dipeptide.

Other degradable linkages that may be included in ADC linkers include ester linkages formed by the reaction of PEG carboxylic acids or activated PEG carboxylic acids with alcohol groups on a biologically active agent, wherein such ester groups generally hydrolyze under physiological conditions to release the biologically active agent. Hydrolytically degradable linkages include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

In certain embodiments, the ADC linker comprises an enzymatically cleavable peptide moiety, for example, an ADC linker comprising structural formula (IVa) or (IVb):

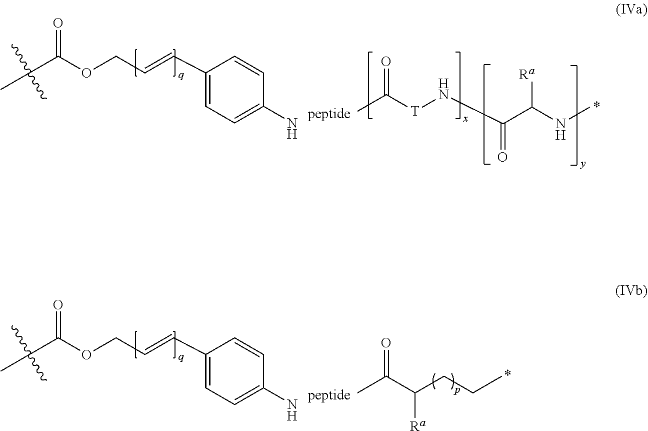

or a salt thereof, wherein: peptide represents a peptide (illustrated C→N and not showing the carboxy and amino “termini”) cleavable by a lysosomal enzyme; T represents a polymer comprising one or more ethylene glycol units or an alkylene chain, or combinations thereof; R^{a} is selected from hydrogen, alkyl, sulfonate and methyl sulfonate; p is an integer ranging from 0 to 5; q is 0 or 1; x is 0 or 1; y is 0 or 1; [symbol] represents the point of attachment of the ADC linker to a cytotoxic and/or cytostatic agent; and * represents the point of attachment to the remainder of the ADC linker.

In certain embodiments, the peptide is selected from a tripeptide or a dipeptide. In particular embodiments, the dipeptide is selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Val-Lys; Ala-Lys; Phe-Cit; Leu-Cit; Ile-Cit; Phe-Arg; and Trp-Cit. In certain embodiments, the dipeptide is selected from: Cit-Val; and Ala-Val.

Specific exemplary embodiments of ADC linkers according to structural formula (IVa) that may be included in the ADCs of the disclosure include the ADC linkers illustrated below (as illustrated, the ADC linkers include a group suitable for covalently linking the ADC linker to a TBM):

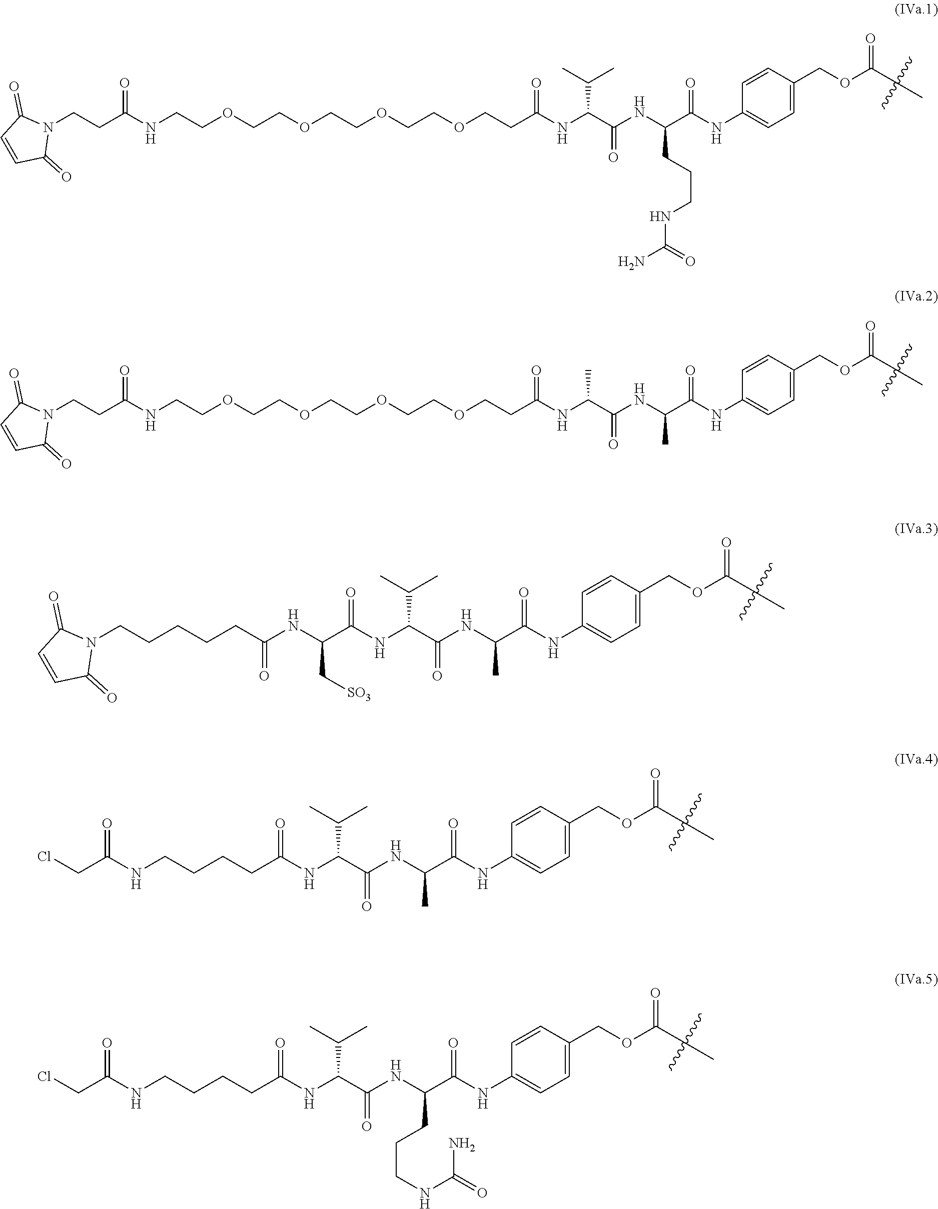

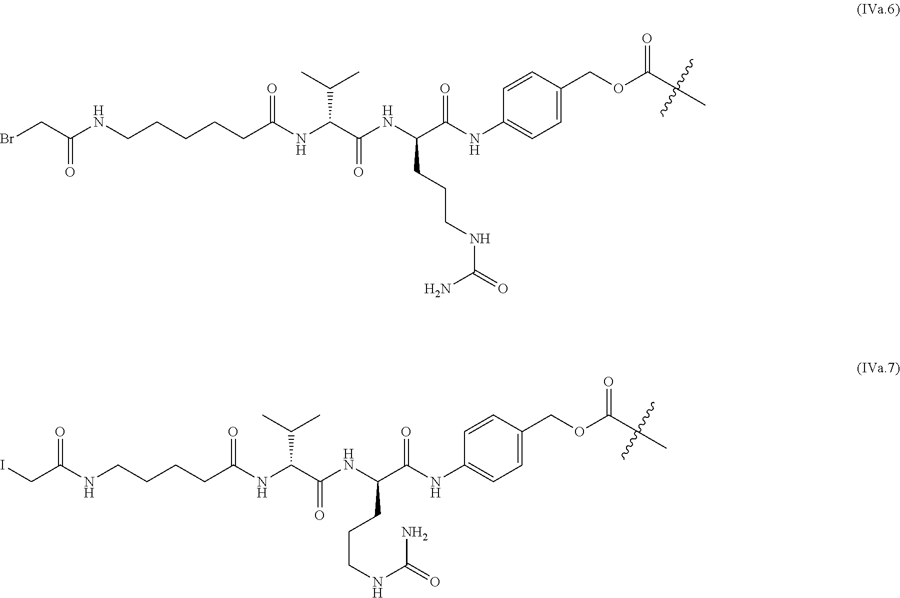 [SMILES: *C(=O)OCc1ccc(NC(=O)[C@@H](CCCNC(N)=O)NC(=O)[C@H](NC(=O)CCCCCNC(=O)CBr)C(C)C)cc1 ; *C(=O)OCc1ccc(NC(=O)[C@@H](CCCNC(N)=O)NC(=O)[C@H](NC(=O)CCCCNC(=O)CI)C(C)C)cc1]

Specific exemplary embodiments of ADC linkers according to structural formula (IVb) that may be included in the ADCs of the disclosure include the ADC linkers illustrated below (as illustrated, the ADC linkers include a group suitable for covalently linking the ADC linker to a TBM):

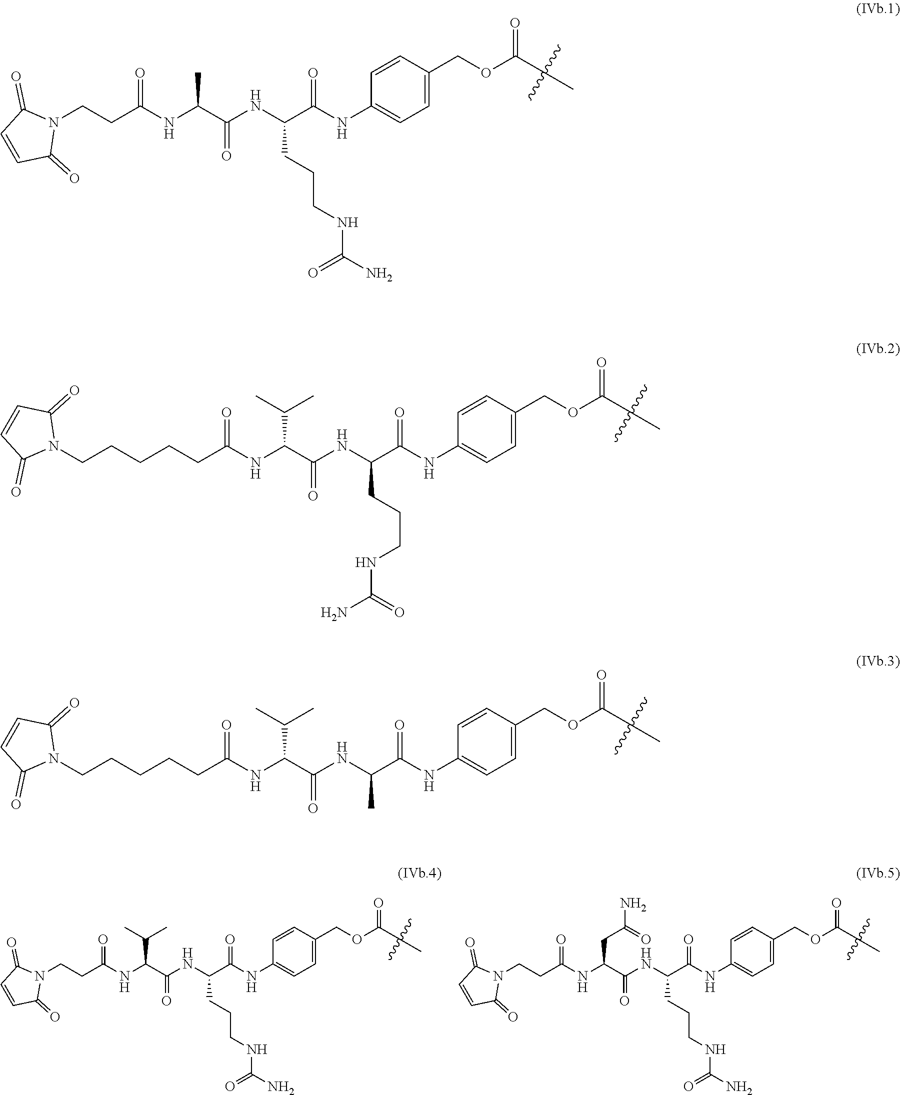 [SMILES: *C(=O)OCc1ccc(NC(=O)[C@H](CCCNC(N)=O)NC(=O)[C@H](C)NC(=O)CCN2C(=O)C=CC2=O)cc1 ; *C(=O)OCc1ccc(NC(=O)[C@@H](CCCNC(N)=O)NC(=O)[C@H](NC(=O)CCCCCN2C(=O)C=CC2=O)C(C)C)cc1 ; *C(=O)OCc1ccc(NC(=O)[C@@H](C)NC(=O)[C@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCN2C(=O)C=CC2=O)C(C)C)cc1 ; *C(=O)OCc1ccc(NC(=O)[C@H](CCCNC(N)=O)NC(=O)[C@H](CC(N)=O)NC(=O)CCN2C(=O)C=CC2=O)cc1]

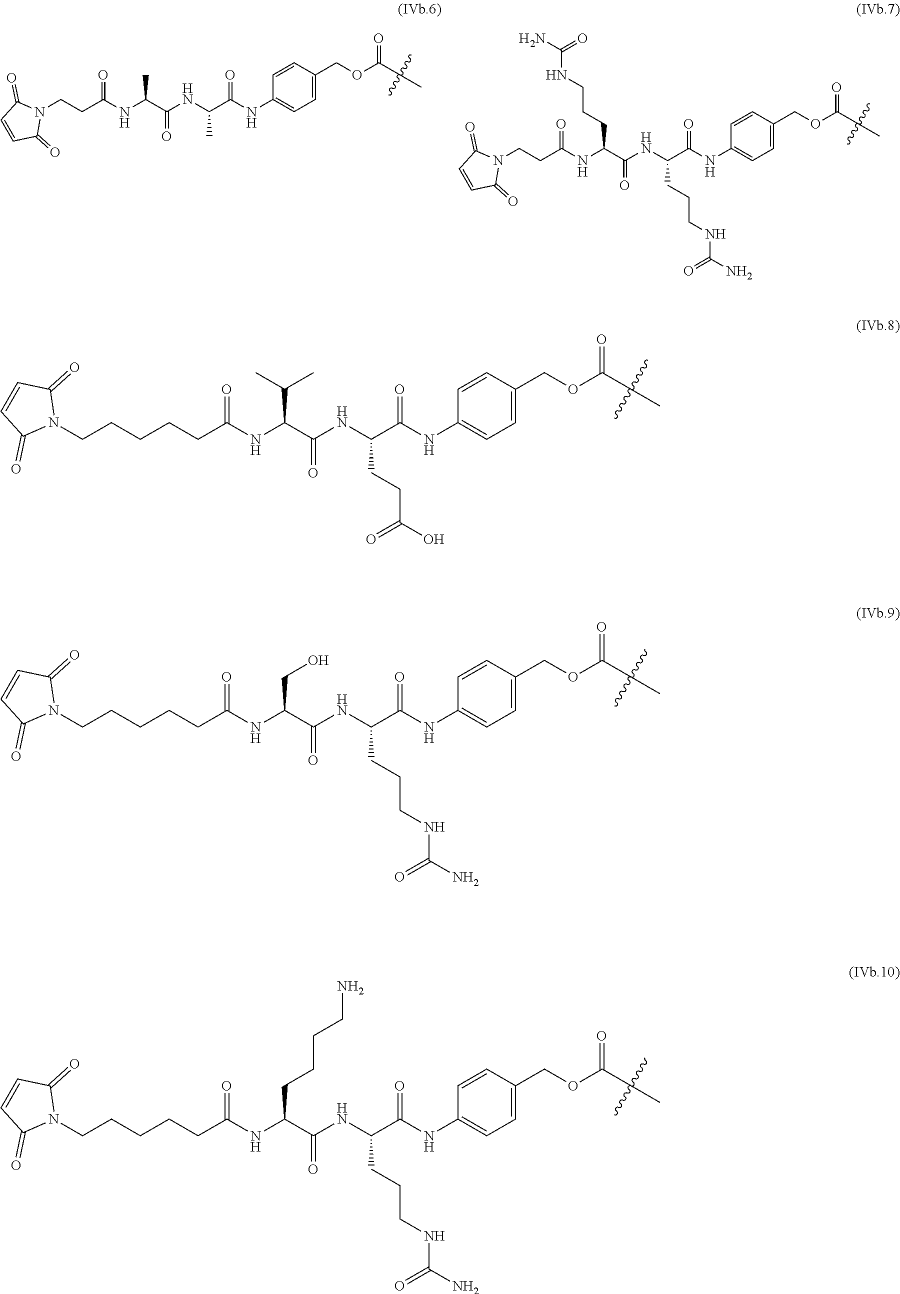 [SMILES: *C(=O)OCc1ccc(NC(=O)[C@H](C)NC(=O)[C@H](C)NC(=O)CCN2C(=O)C=CC2=O)cc1 ; *C(=O)OCc1ccc(NC(=O)[C@H](CCCNC(N)=O)NC(=O)[C@H](CCCNC(N)=O)NC(=O)CCN2C(=O)C=CC2=O)cc1 ; *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1]

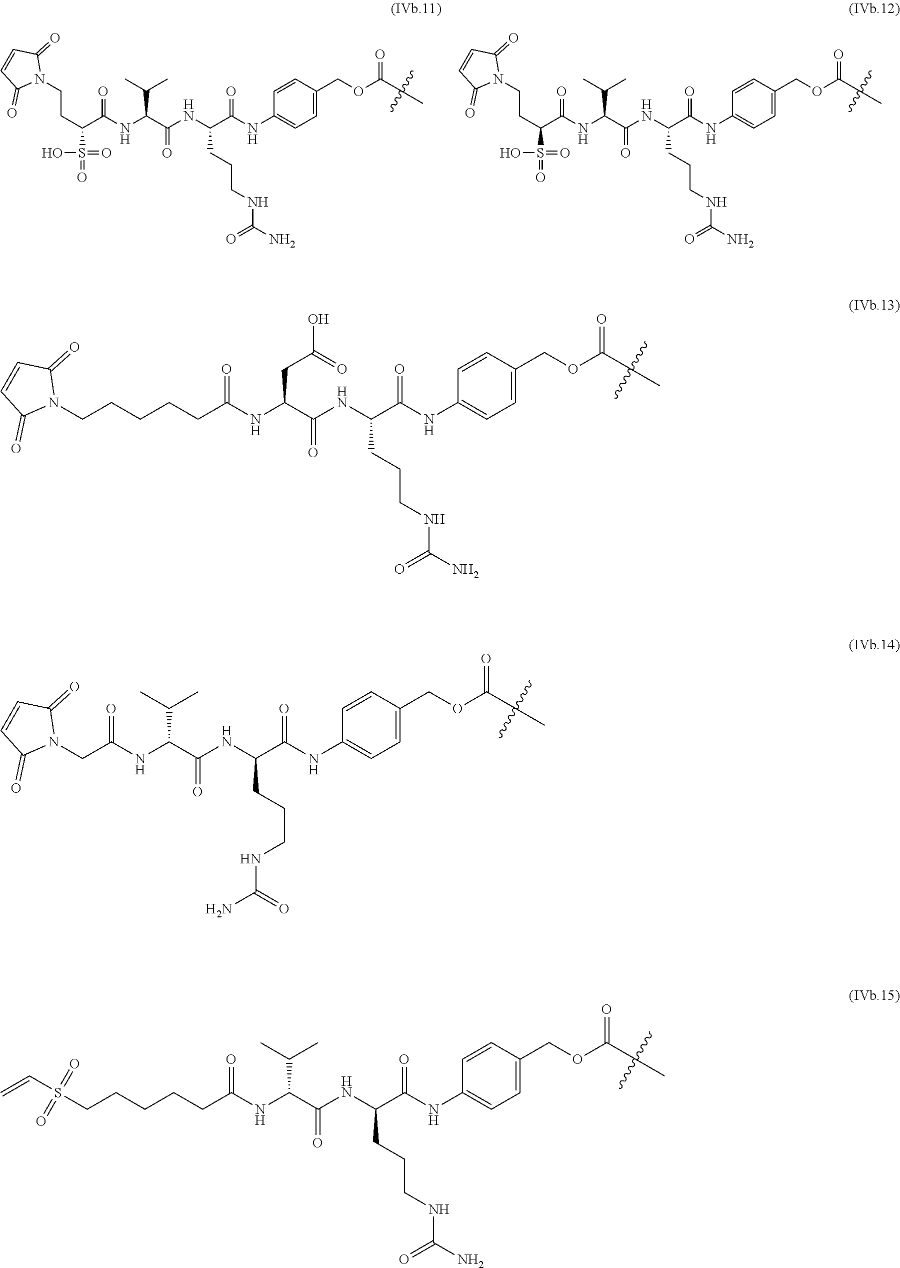 [SMILES: *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1 ; *C(=O)OCc1ccc(NC(=O)[C@H](CCCNC(N)=O)NC(=O)[C@H](CC(=O)O)NC(=O)CCCCCN2C(=O)C=CC2=O)cc1 ; *C(=O)OCc1ccc(NC(=O)[C@@H](CCCNC(N)=O)NC(=O)[C@H](NC(=O)CN2C(=O)C=CC2=O)C(C)C)cc1 ; *C(=O)OCc1ccc(NC(=O)[C@@H](CCCNC(N)=O)NC(=O)[C@H](NC(=O)CCCCCS(=O)(=O)C=C)C(C)C)cc1]

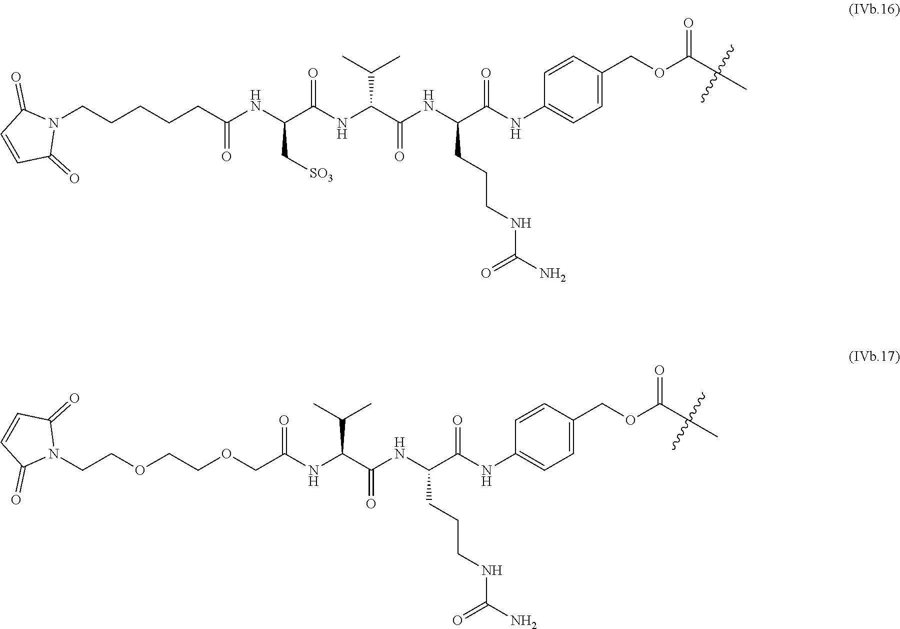

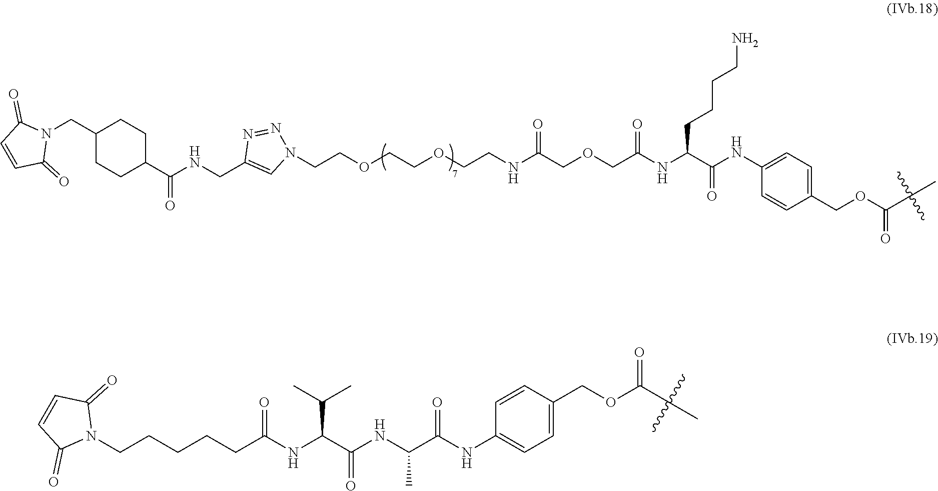

In certain embodiments, the ADC linker comprises an enzymatically cleavable peptide moiety, for example, an ADC linker comprising structural formula (IVc) or (IVd):

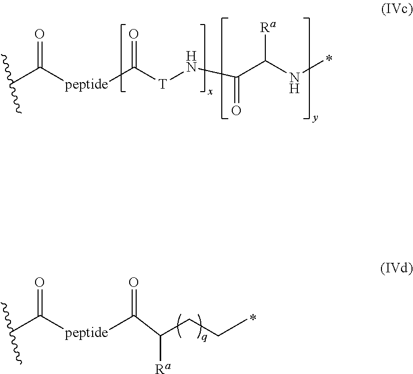

or a salt thereof, wherein: peptide represents a peptide (illustrated C→N and not showing the carboxy and amino “termini”) cleavable by a lysosomal enzyme; T represents a polymer comprising one or more ethylene glycol units or an alkylene chain, or combinations thereof; Ra is selected from hydrogen, alkyl, sulfonate and methyl sulfonate; p is an integer ranging from 0 to 5; q is 0 or 1; x is 0 or 1; y is 0 or 1; [symbol] represents the point of attachment of the ADC linker to a cytotoxic and/or cytostatic agent; and * represents the point of attachment to the remainder of the ADC linker.

Specific exemplary embodiments of ADC linkers according to structural formula (IVc) that may be included in the ADCs of the disclosure include the ADC linkers illustrated below (as illustrated, the ADC linkers include a group suitable for covalently linking the ADC linker to a TBM):

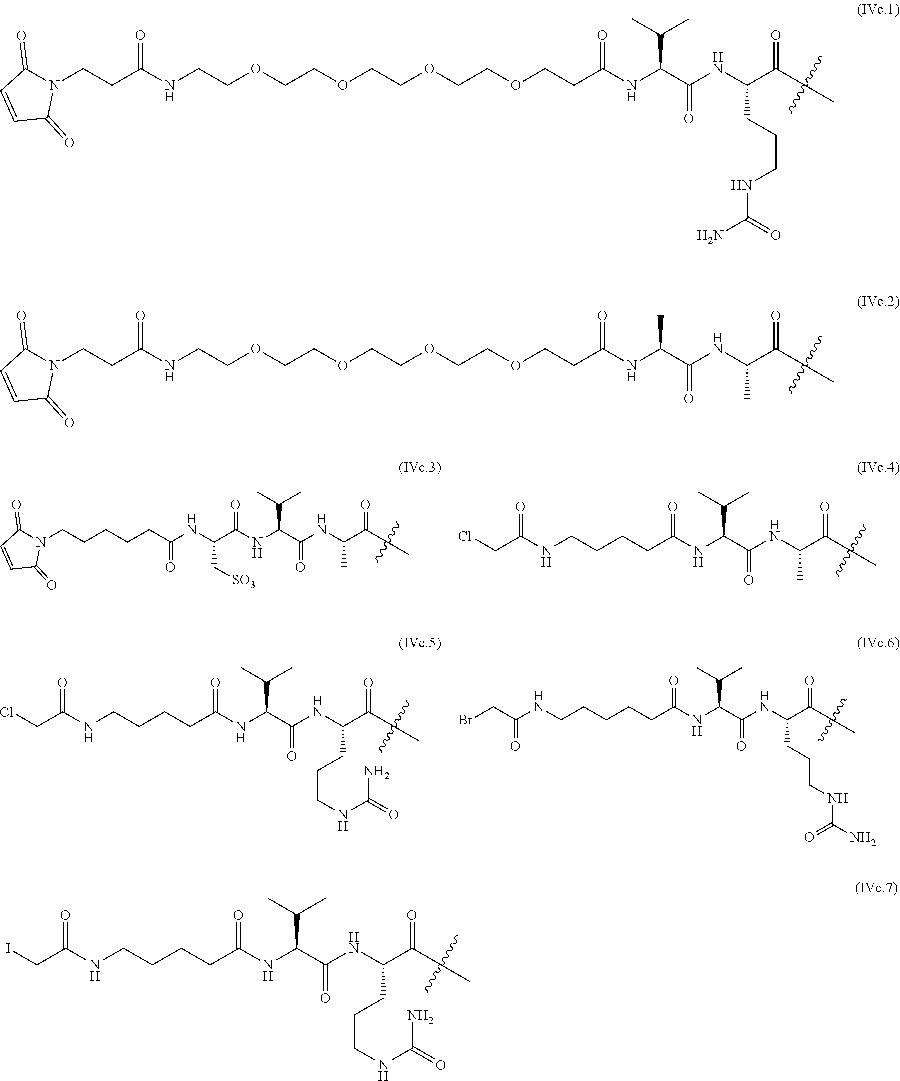

Specific exemplary embodiments of ADC linkers according to structural formula (IVd) that may be included in the ADCs of the disclosure include the ADC linkers illustrated below (as illustrated, the ADC linkers include a group suitable for covalently linking the ADC linker to a TBM):

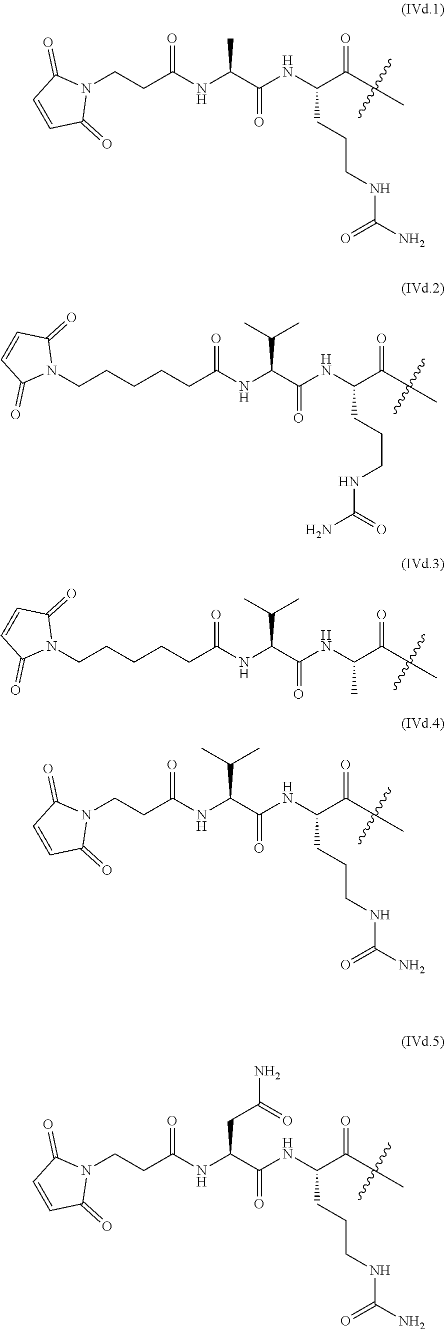 [SMILES: *C(=O)[C@H](CCCNC(N)=O)NC(=O)[C@H](C)NC(=O)CCN1C(=O)C=CC1=O ; *C(=O)[C@H](CCCNC(N)=O)NC(=O)[C@@H](NC(=O)CCCCCN1C(=O)C=CC1=O)C(C)C ; *C(=O)[C@H](C)NC(=O)[C@@H](NC(=O)CCCCCN1C(=O)C=CC1=O)C(C)C ; *C(=O)[C@H](CCCNC(N)=O)NC(=O)[C@@H](NC(=O)CCN1C(=O)C=CC1=O)C(C)C ; *C(=O)[C@H](CCCNC(N)=O)NC(=O)[C@H](CC(N)=O)NC(=O)CCN1C(=O)C=CC1=O]

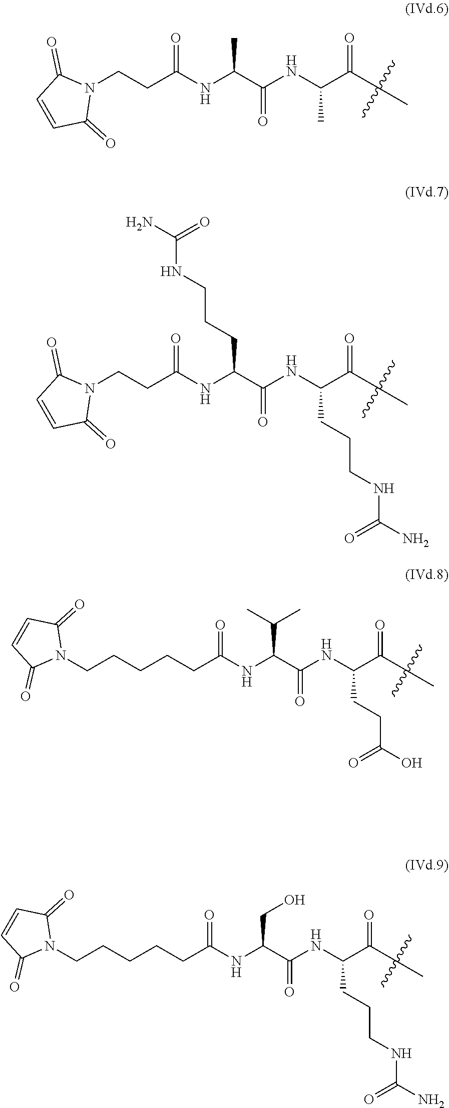 [SMILES: *C(=O)[C@H](C)NC(=O)[C@H](C)NC(=O)CCN1C(=O)C=CC1=O ; *C(=O)[C@H](CCCNC(N)=O)NC(=O)[C@H](CCCNC(N)=O)NC(=O)CCN1C(=O)C=CC1=O ; *C(=O)[C@H](CCC(=O)O)NC(=O)[C@@H](NC(=O)CCCCCN1C(=O)C=CC1=O)C(C)C ; *C(=O)[C@H](CCCNC(N)=O)NC(=O)[C@H](CO)NC(=O)CCCCCN1C(=O)C=CC1=O]

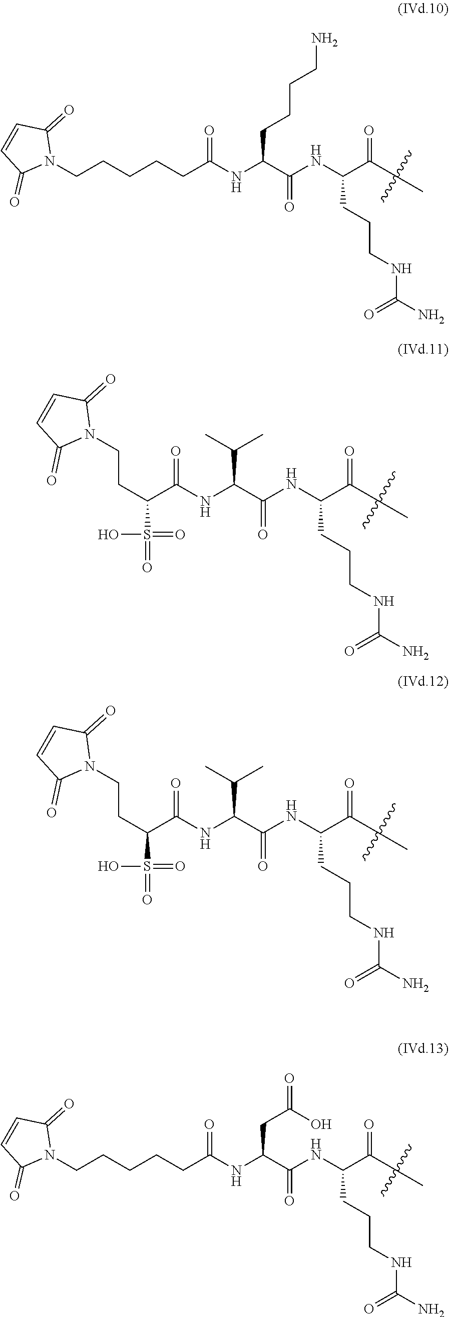 [SMILES: *C(=O)[C@H](CCCNC(N)=O)NC(=O)[C@H](CCCCN)NC(=O)CCCCCN1C(=O)C=CC1=O ; *C(=O)[C@H](CCCNC(N)=O)NC(=O)[C@@H](NC(=O)[C@@H](CCN1C(=O)C=CC1=O)S(=O)(=O)O)C(C)C ; *C(=O)[C@H](CCCNC(N)=O)NC(=O)[C@@H](NC(=O)[C@H](CCN1C(=O)C=CC1=O)S(=O)(=O)O)C(C)C ; *C(=O)[C@H](CCCNC(N)=O)NC(=O)[C@H](CC(=O)O)NC(=O)CCCCCN1C(=O)C=CC1=O]

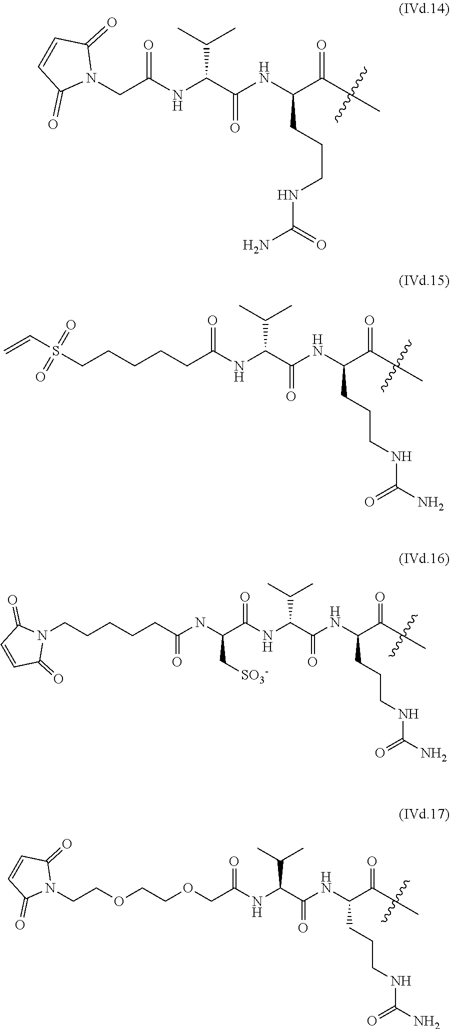

In certain embodiments, the ADC linker comprising structural formula (IVa), (IVb), (IVc), or (IVd) further comprises a carbonate moiety cleavable by exposure to an acidic medium. In particular embodiments, the ADC linker is attached through an oxygen to a cytotoxic and/or cytostatic agent.

6.9.2.2. Non-Cleavable Linkers

Although cleavable ADC linkers may provide certain advantages, the ADC linkers comprising the ADCs of the disclosure need not be cleavable. For noncleavable ADC linkers, the release of drug does not depend on the differential properties between the plasma and some cytoplasmic compartments. The release of the drug is postulated to occur after internalization of the ADC via antigen-mediated endocytosis and delivery to lysosomal compartment, where the TBM is degraded to the level of amino acids through intracellular proteolytic degradation. This process releases a drug derivative, which is formed by the drug, the ADC linker, and the amino acid residue to which the ADC linker was covalently attached. The amino acid drug metabolites from conjugates with noncleavable ADC linkers are more hydrophilic and generally less membrane permeable, which leads to less bystander effects and less nonspecific toxicities compared to conjugates with a cleavable ADC linker. In general, ADCs with noncleavable ADC linkers have greater stability in circulation than ADCs with cleavable ADC linkers. Non-cleavable ADC linkers may be alkylene chains, or maybe polymeric in natures, such as, for example, based upon polyalkylene glycol polymers, amide polymers, or may include segments of alkylene chains, polyalkylene glocols and/or amide polymers.

A variety of non-cleavable ADC linkers used to link drugs to TBMs have been described. See, Jeffrey et al., 2006, Bioconjug. Chem. 17; 831-840; Jeffrey et al., 2007, Bioorg. Med. Chem. Lett. 17:2278-2280; and Jiang et al., 2005, J. Am. Chem. Soc. 127:11254-11255, each of which is incorporated herein by reference. All of these ADC linkers may be included in the ADCs of the disclosure.

In certain embodiments, the ADC linker is non-cleavable in vivo, for example an ADC linker according to structural formula (VIa), (VIb), (VIc) or (VId) (as illustrated, the ADC linkers include a group suitable for covalently linking the ADC linker to a TBM:

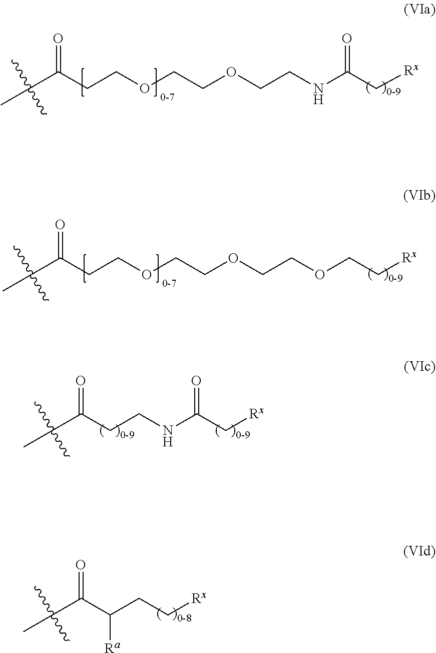

or salts thereof, wherein: R^{a} is selected from hydrogen, alkyl, sulfonate and methyl sulfonate; R^{x} is a moiety including a functional group capable of covalently linking the ADC linker to a TBM; and [symbol] represents the point of attachment of the ADC linker to a cytotoxic and/or cytostatic agent.

Specific exemplary embodiments of ADC linkers according to structural formula (VIa)-(VId) that may be included in the ADCs of the disclosure include the ADC linkers illustrated below (as illustrated, the ADC linkers include a group suitable for covalently linking the ADC linker to a TBM, and [symbol] represents the point of attachment to a cytotoxic and/or cytostatic agent):

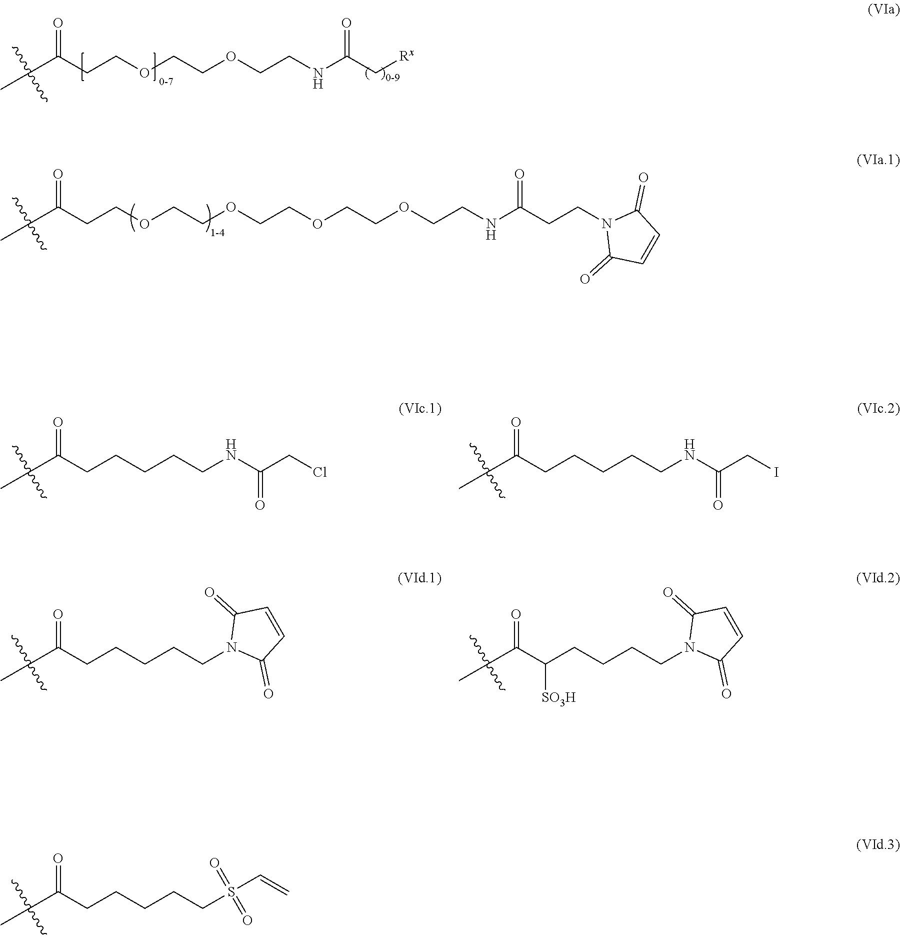

6.9.2.3. Groups Used to Attach Linkers to TBMs

A variety of groups may be used to attach ADC linker-drug synthons to TBMs to yield ADCs. Attachment groups can be electrophilic in nature and include: maleimide groups, activated disulfides, active esters such as NHS esters and HOBt esters, haloformates, acid halides, alkyl and benzyl halides such as haloacetamides. As discussed below, there are also emerging technologies related to “self-stabilizing” maleimides and “bridging disulfides” that can be used in accordance with the disclosure. The specific group used will depend, in part, on the site of attachment to the TBM.

One example of a “self-stabilizing” maleimide group that hydrolyzes spontaneously under TBM conjugation conditions to give an ADC species with improved stability is depicted in the schematic below. See US20130309256 A1; also Lyon et al., Nature Biotech published online, doi:10.1038/nbt.2968.

Normal System:

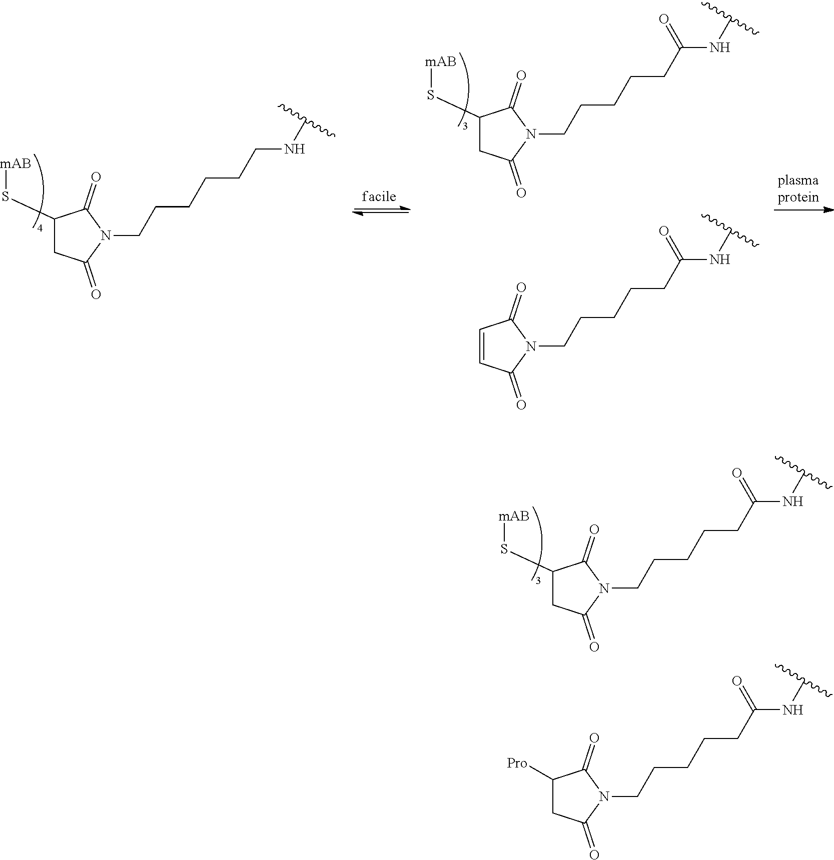

Leads to “DAR loss” over time

SGN MaIDPR (maleimido dipropylamino) system:

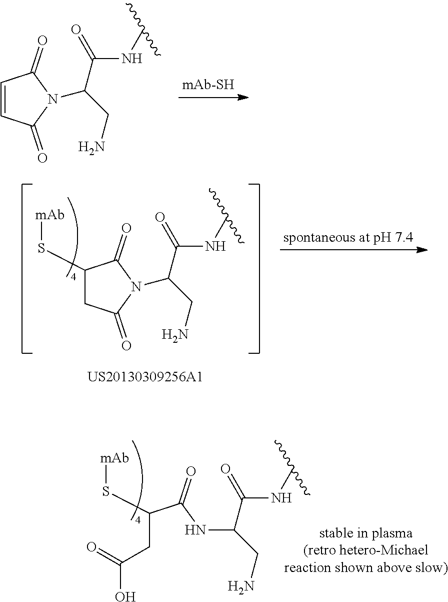

Polytherics has disclosed a method for bridging a pair of sulfhydryl groups derived from reduction of a native hinge disulfide bond. See, Badescu et al., 2014, Bioconjugate Chem. 25:1124-1136. The reaction is depicted in the schematic below. An advantage of this methodology is the ability to synthesize enriched DAR4 ADCs by full reduction of IgGs (to give 4 pairs of sulfhydryls) followed by reaction with 4 equivalents of the alkylating agent. ADCs containing “bridged disulfides” are also said to have increased stability.

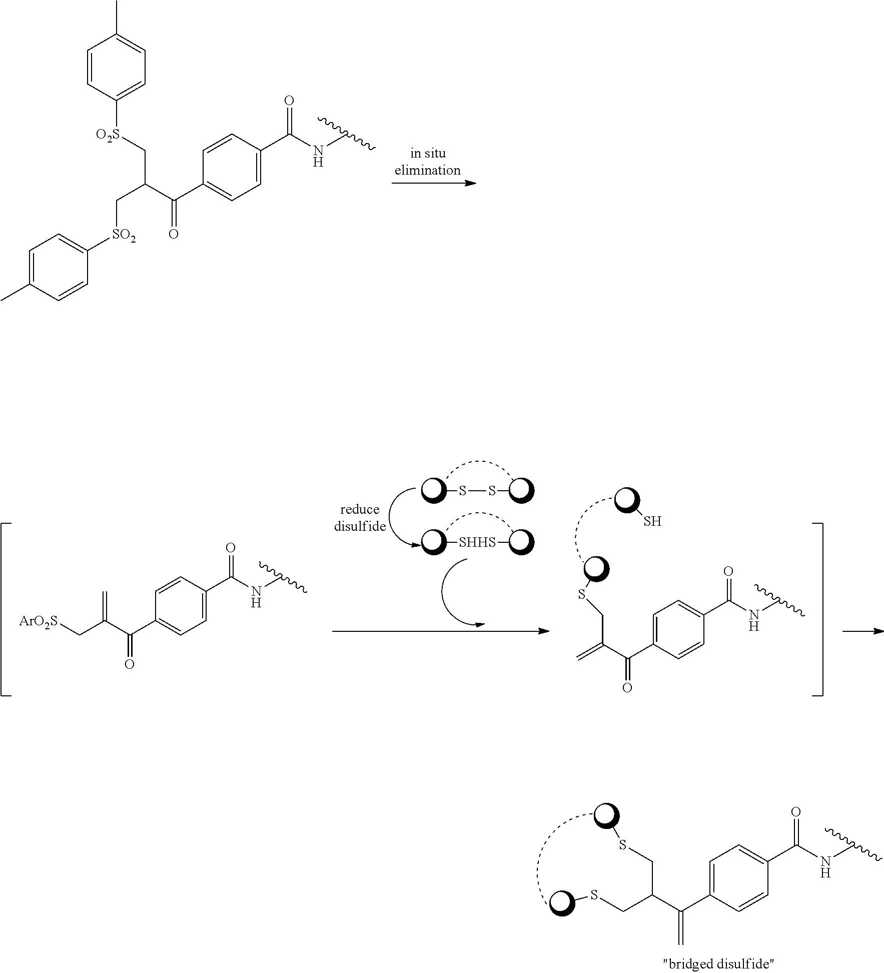

Similarly, as depicted below, a maleimide derivative (1, below) that is capable of bridging a pair of sulfhydryl groups has been developed. See WO2013/085925.

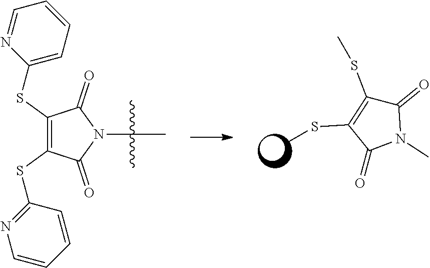

6.9.2.4. ADC Linker Selection Considerations

As is known by skilled artisans, the ADC linker selected for a particular ADC may be influenced by a variety of factors, including but not limited to, the site of attachment to the TBM (e.g., lys, cys or other amino acid residues), structural constraints of the drug pharmacophore and the lipophilicity of the drug. The specific ADC linker selected for an ADC should seek to balance these different factors for the specific TBM/drug combination. For a review of the factors that are influenced by choice of ADC linkers in ADCs, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013.

For example, ADCs have been observed to effect killing of bystander antigen-negative cells present in the vicinity of the antigen-positive tumor cells. The mechanism of bystander cell killing by ADCs has indicated that metabolic products formed during intracellular processing of the ADCs may play a role. Neutral cytotoxic metabolites generated by metabolism of the ADCs in antigen-positive cells appear to play a role in bystander cell killing while charged metabolites may be prevented from diffusing across the membrane into the medium and therefore cannot affect bystander killing. In certain embodiments, the ADC linker is selected to attenuate the bystander killing effect caused by cellular metabolites of the ADC. In certain embodiments, the ADC linker is selected to increase the bystander killing effect.

The properties of the ADC linker may also impact aggregation of the ADC under conditions of use and/or storage. Typically, ADCs reported in the literature contain no more than 3-4 drug molecules per antibody molecule (see, e.g., Chari, 2008, Acc Chem Res 41:98-107). Attempts to obtain higher drug-to-antibody ratios (“DAR”) often failed, particularly if both the drug and the ADC linker were hydrophobic, due to aggregation of the ADC (King et al., 2002, J Med Chem 45:4336-4343; Hollander et al., 2008, Bioconjugate Chem 19:358-361; Burke et al., 2009 Bioconjugate Chem 20:1242-1250). In many instances, DARs higher than 3-4 could be beneficial as a means of increasing potency. In instances where the cytotoxic and/or cytostatic agent is hydrophobic in nature, it may be desirable to select ADC linkers that are relatively hydrophilic as a means of reducing ADC aggregation, especially in instances where DARS greater than 3-4 are desired. Thus, in certain embodiments, the ADC linker incorporates chemical moieties that reduce aggregation of the ADCs during storage and/or use. An ADC linker may incorporate polar or hydrophilic groups such as charged groups or groups that become charged under physiological pH to reduce the aggregation of the ADCs. For example, an ADC linker may incorporate charged groups such as salts or groups that deprotonate, e.g., carboxylates, or protonate, e.g., amines, at physiological pH.

Exemplary polyvalent ADC linkers that have been reported to yield DARs as high as 20 that may be used to link numerous cytotoxic and/or cytostatic agents to a TBM are described in WO 2009/073445; WO 2010/068795; WO 2010/138719; WO 2011/120053; WO 2011/171020; WO 2013/096901; WO 2014/008375; WO 2014/093379; WO 2014/093394; WO 2014/093640, the content of which are incorporated herein by reference in their entireties.

In particular embodiments, the aggregation of the ADCs during storage or use is less than about 10% as determined by size-exclusion chromatography (SEC). In particular embodiments, the aggregation of the ADCs during storage or use is less than 10%, such as less than about 5%, less than about 4%, less than about 3%, less than about 2%, less than about 1%, less than about 0.5%, less than about 0.1%, or even lower, as determined by size-exclusion chromatography (SEC).

6.9.3. Methods of Making ADCs

The ADCs of the disclosure may be synthesized using chemistries that are well-known. The chemistries selected will depend upon, among other things, the identity of the cytotoxic and/or cytostatic agent(s), the ADC linker and the groups used to attach ADC linker to the TBM. Generally, ADCs according to formula (I) may be prepared according to the following scheme:

D-L-R^{x}+Ab-R^{y}→[D-L-XY]ₙ-Ab (I)

where D, L, Ab, XY and n are as previously defined, and R^{x} and R^{y} represent complementary groups capable of forming a covalent linkages with one another, as discussed above.

The identities of groups R^{x} and R^{y} will depend upon the chemistry used to link synthon D-L-R^{x} to the TBM. Generally, the chemistry used should not alter the integrity of the TBM, for example its ability to bind its target. Preferably, the binding properties of the conjugated antibody will closely resemble those of the unconjugated TBM. A variety of chemistries and techniques for conjugating molecules to biological molecules and in particular to immunoglobulins, whose components are typically building blocks of the TBMs of the disclosure, are well-known. See, e.g., Amon et al., “Monoclonal Antibodies For Immunotargeting Of Drugs In Cancer Therapy,” in: Monoclonal Antibodies And Cancer Therapy, Reisfeld et al. Eds., Alan R. Liss, Inc., 1985; Hellstrom et al., “Antibodies For Drug Delivery,” in: Controlled Drug Delivery, Robinson et al. Eds., Marcel Dekker, Inc., 2nd Ed. 1987; Thorpe, “Antibody Carriers Of Cytotoxic Agents In Cancer Therapy: A Review,” in: Monoclonal Antibodies '84: Biological And Clinical Applications, Pinchera et al., Eds., 1985; “Analysis, Results, and Future Prospective of the Therapeutic Use of Radiolabeled Antibody In Cancer Therapy,” in: Monoclonal Antibodies For Cancer Detection And Therapy, Baldwin et al., Eds., Academic Press, 1985; Thorpe et al., 1982, Immunol. Rev. 62:119-58; PCT publication WO 89/12624. Any of these chemistries may be used to link the synthons to a TBM.

A number of functional groups R^{x} and chemistries useful for linking synthons to accessible lysine residues are known, and include by way of example and not limitation NHS-esters and isothiocyanates.

A number of functional groups R^{x} and chemistries useful for linking synthons to accessible free sulfhydryl groups of cysteine residues are known, and include by way of example and not limitation haloacetyls and maleimides.

However, conjugation chemistries are not limited to available side chain groups. Side chains such as amines may be converted to other useful groups, such as hydroxyls, by linking an appropriate small molecule to the amine. This strategy can be used to increase the number of available linking sites on the antibody by conjugating multifunctional small molecules to side chains of accessible amino acid residues of the TBM. Functional groups R^{x} suitable for covalently linking the synthons to these “converted” functional groups are then included in the synthons.

The TBM may also be engineered to include amino acid residues for conjugation. An approach for engineering TBMs to include non-genetically encoded amino acid residues useful for conjugating drugs in the context of ADCs is described by Axup et al., 2012, Proc Natl Acad Sci USA. 109(40):16101-16106, as are chemistries and functional group useful for linking synthons to the non-encoded amino acids.

Typically, the synthons are linked to the side chains of amino acid residues of the TBM, including, for example, the primary amino group of accessible lysine residues or the sulfhydryl group of accessible cysteine residues. Free sulfhydryl groups may be obtained by reducing interchain disulfide bonds.

For linkages where R^{y} is a sulfhydryl group (for example, when R^{x} is a maleimide), the TBM is generally first fully or partially reduced to disrupt interchain disulfide bridges between cysteine residues.

Cysteine residues that do not participate in disulfide bridges may engineered into a TBM by modification of one or more codons. Reducing these unpaired cysteines yields a sulfhydryl group suitable for conjugation. Preferred positions for incorporating engineered cysteines include, by way of example and not limitation, positions S112C, S113C, A114C, S115C, A176C, 5180C, S252C, V286C, V292C, S357C, A359C, S398C, S428C (Kabat numbering) on the human IgG₁ heavy chain and positions V110C, S114C, S121C, S127C, S168C, V205C (Kabat numbering) on the human Ig kappa light chain (see, e.g., U.S. Pat. Nos. 7,521,541, 7,855,275 and 8,455,622).

As will appreciated by skilled artisans, the number of cytotoxic and/or cytostatic agents linked to a TBM molecule may vary, such that a collection of ADCs may be heterogeneous in nature, where some TBMs contain one linked agent, some two, some three, etc. (and some none). The degree of heterogeneity will depend upon, among other things, the chemistries used for linking the cytotoxic and/or cytostatic agents. For example, where the TBMs are reduced to yield sulfhydryl groups for attachment, heterogeneous mixtures of TBMs having zero, 2, 4, 6 or 8 linked agents per molecule are often produced. Furthermore, by limiting the molar ratio of attachment compound, TBMs having zero, 1, 2, 3, 4, 5, 6, 7 or 8 linked agents per molecule are often produced. Thus, it will be understood that depending upon context, stated drug TBM ratios (DTRs) may be averages for a collection of TBMs. For example, “DTR4” can refer to an ADC preparation that has not been subjected to purification to isolate specific DTR peaks and can comprise a heterogeneous mixture of ADC molecules having different numbers of cytostatic and/or cytotoxic agents attached per TBM (e.g., 0, 2, 4, 6, 8 agents per TBM), but has an average drug-to-TBM ratio of 4. Similarly, in some embodiments, “DTR2” refers to a heterogeneous ADC preparation in which the average drug-to-TBM ratio is 2.

When enriched preparations are desired, TBMs having defined numbers of linked cytotoxic and/or cytostatic agents may be obtained via purification of heterogeneous mixtures, for example, via column chromatography, e.g., hydrophobic interaction chromatography.

Purity may be assessed by a variety of methods, as is known in the art. As a specific example, an ADC preparation may be analyzed via HPLC or other chromatography and the purity assessed by analyzing areas under the curves of the resultant peaks.

### 6.10. Preparations

The disclosure provides preparations comprising a plurality of TBMs and/or a plurality of TBM conjugates, e.g., at least 100, at least 1,000, at least 10,000, or at least 100,000 TBMs and/or TBM conjugates. Preparations include, for example, cell culture supernatants comprising TBM molecules and compositions of enriched or purified TBM molecules (e.g., TBMs fractionated or purified from a cell culture supernatant).

Preparations can comprise, for example, a plurality of TBMs or conjugates wherein at least 50% (e.g., at least 50%, at least 60%, at least 70%, at least 80%, at least 90%, at least 95%, at least 97%, at least 98%, at least 99%) of the trispecific molecules in the preparation have the same primary amino acid sequence. In various embodiments, 50% to 99% of the trispecific molecules in a preparation have the same primary amino acid sequence (e.g., 50% to 95%, 50% to 80%, 50% to 70%, 60% to 95%, 60% to 80%, 60% to 70%, 70% to 95%, 70% to 80%, 80% to 95% 95% to 99%, or any range bounded by any two of the foregoing values).

In some embodiments, a majority of the the trispecific molecules in a preparation have the same interchain crosslinks (e.g., at least 50%, at least 60%, at least 70%, at least 80%, at least 90%, at least 95%, at least 97%, at least 98%, or at least 99%). As used herein, an “interchain crosslink” refers to a crosslink between two linear polypeptide chains, e.g., crosslinks created by disulfide bridges. In various embodiments, 50% to 99% of the trispecific molecules in a preparation have the same interchain crosslinks (e.g., 50% to 95%, 50% to 80%, 50% to 70%, 60% to 95%, 60% to 80%, 60% to 70%, 70% to 95%, 70% to 80%, 80% to 95% 95% to 99%, or any range bounded by any two of the foregoing values).

In some embodiments, a majority of the the trispecific molecules in a preparation have the same ABM1:ABM2:ABM3 ratio (e.g., at least 50%, at least 60%, at least 70%, at least 80%, at least 90%, at least 95%, at least 97%, at least 98%, or at least 99%). In various embodiments, 50% to 99% of the trispecific molecules in a preparation have the same ABM1:ABM2:ABM3 ratio (e.g., 50% to 95%, 50% to 80%, 50% to 70%, 60% to 95%, 60% to 80%, 60% to 70%, 70% to 95%, 70% to 80%, 80% to 95% 95% to 99%, or any range bounded by any two of the foregoing values).

### 6.11. Pharmaceutical Compositions

The TBMs of the disclosure (as well as their conjugates; references to TBMs in this disclosure also refers to conjugates comprising the TBMs, such as ADCs, unless the context dictates otherwise) can be formulated as pharmaceutical compositions comprising the TBMs, for example containing one or more pharmaceutically acceptable excipients or carriers. To prepare pharmaceutical or sterile compositions comprising the TBMs of the present disclosure a TBM preparation can be combined with one or more pharmaceutically acceptable excipient or carrier.

For example, formulations of TBMs can be prepared by mixing TBMs with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

Selecting an administration regimen for a TBM depends on several factors, including the serum or tissue turnover rate of the TBM, the level of symptoms, the immunogenicity of the TBM, and the accessibility of the target cells. In certain embodiments, an administration regimen maximizes the amount of TBM delivered to the subject consistent with an acceptable level of side effects. Accordingly, the amount of TBM delivered depends in part on the particular TBM and the severity of the condition being treated. Guidance in selecting appropriate doses of antibodies and small molecules are available (see, e.g., Wawrzynczak, 1996, Antibody Therapy, Bios Scientific Pub. Ltd, Oxfordshire, UK; Kresina (ed.), 1991, Monoclonal Antibodies, Cytokines and Arthritis, Marcel Dekker, New York, N.Y.; Bach (ed.), 1993, Monoclonal Antibodies and Peptide Therapy in Autoimmune Diseases, Marcel Dekker, New York, N.Y.; Baert et al., 2003, New Engl. J. Med. 348:601-608; Milgrom et al., 1999, New Engl. J. Med. 341:1966-1973; Slamon et al., 2001, New Engl. J. Med. 344:783-792; Beniaminovitz et al., 2000, New Engl. J. Med. 342:613-619; Ghosh et al., 2003, New Engl. J. Med. 348:24-32; Lipsky et al., 2000, New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the TBMs in the pharmaceutical compositions of the present disclosure may be varied so as to obtain an amount of the TBM which is effective to achieve the desired therapeutic response for a particular subject, composition, and mode of administration, without being toxic to the subject. The selected dosage level will depend upon a variety of pharmacokinetic factors including the activity of the particular TBM, the route of administration, the time of administration, the rate of excretion of the particular TBM being employed, the duration of the treatment, other agents (e.g., active agents such as therapeutic drugs or compounds and/or inert materials used as carriers) in combination with the particular TBM employed, the age, sex, weight, condition, general health and prior medical history of the subject being treated, and like factors known in the medical arts.

Compositions comprising the TBMs of the disclosure can be provided by continuous infusion, or by doses at intervals of, e.g., one day, one week, or 1-7 times per week. Doses can be provided intravenously, subcutaneously, topically, orally, nasally, rectally, intramuscular, intracerebrally, or by inhalation. A specific dose protocol is one involving the maximal dose or dose frequency that avoids significant undesirable side effects.

An effective amount for a particular subject may vary depending on factors such as the condition being treated, the overall health of the subject, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., 1983, Biopolymers 22:547-556; Langer et al., 1981, J. Biomed. Mater. Res. 15:167-277; Langer, 1982, Chem. Tech. 12:98-105; Epstein et al., 1985, Proc. Natl. Acad. Sci. USA 82:3688-3692; Hwang et al., 1980, Proc. Natl. Acad. Sci. USA 77:4030-4034; U.S. Pat. Nos. 6,350,466 and 6,316,024). Where necessary, the composition may also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

A composition of the present disclosure may also be administered via one or more routes of administration using one or more of a variety of methods known in the art. As will be appreciated by the skilled artisan, the route and/or mode of administration will vary depending upon the desired results. Selected routes of administration for TBMs include intravenous, intramuscular, intradermal, intraperitoneal, subcutaneous, spinal or other general routes of administration, for example by injection or infusion. General administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-general route, such as a topical, epidermal or mucosal route of administration, for example, intranasally, orally, vaginally, rectally, sublingually or topically. In one embodiment, the TBMs is administered by infusion. In another embodiment, the multispecific epitope binding protein of the disclosure is administered subcutaneously.

If the TBMs are administered in a controlled release or sustained release system, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). Polymeric materials can be used to achieve controlled or sustained release of the therapies of the disclosure (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more TBMs of the disclosure. See, e.g., U.S. Pat. No. 4,526,938, PCT publication WO 91/05548, PCT publication WO 96/20698, Ning et al., 1996, Radiotherapy & Oncology 39:179-189, Song et al., 1995, PDA Journal of Pharmaceutical Science & Technology 50:372-397, Cleek et al., 1997, Pro. Intl Symp. Control. Rel. Bioact. Mater. 24:853-854, and Lam et al., 1997, Proc. Intl Symp. Control Rel. Bioact. Mater. 24:759-760, each of which is incorporated herein by reference in their entirety.

If the TBMs are administered topically, they can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the compositions comprising the TBMs are administered intranasally, the TBMs can be formulated in an aerosol form, spray, mist or in the form of drops. In particular, prophylactic or therapeutic agents for use according to the present disclosure can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

The TBMs of the disclosure can be administered in combination therapy regimens, as described in Section 6.13, infra.

In certain embodiments, the TBMs can be formulated to ensure proper distribution in vivo. For example, the blood-brain barrier (BBB) excludes many highly hydrophilic compounds. To ensure that the therapeutic compounds of the disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade, 1989, J. Clin. Pharmacol. 29:685). Exemplary targeting moieties include folate or biotin (see, e.g., U.S. Pat. No. 5,416,016 to Low et al.); mannosides (Umezawa et al., 1988, Biochem. Biophys. Res. Commun. 153:1038); antibodies (Bloeman et al., 1995, FEBS Lett. 357:140; Owais et al., 1995, Antimicrob. Agents Chemother. 39:180); surfactant protein A receptor (Briscoe et al., 1995, Am. J. Physiol. 1233:134); p 120 (Schreier et al., 1994, J. Biol. Chem. 269:9090); see also Keinanen and Laukkanen, 1994, FEBS Lett. 346:123; Killion and Fidler, 1994, Immunomethods 4:273.

When used in combination therapy, e.g., as described in Section 6.13, infra, a TBM of the disclosure and one or more additional agents can be administered to a subject in the same pharmaceutical composition. Alternatively, the TBM and the additional agent(s) of the combination therapies can be administered concurrently to a subject in separate pharmaceutical compositions.

The therapeutic methods described herein may further comprise carrying a “companion diagnostic” test whereby a sample from a subject who is a candidate for therapy with a TBM of the disclosure is tested for the expression of the TAA targeted by ABM3. The companion diagnostic test can be performed prior to initiating therapy with a TBM of the disclosure and/or during a therapeutic regimen with a TBM of the disclosure to monitor the subject's continued suitability for TBM therapy. The agent used in the companion diagnostic can be the TBM itself or another diagnostic agent, for example a labeled monospecific antibody against the TAA recognized by ABM3 or a nucleic acid probe to detect TAA RNA. The sample that can be tested in a companion diagnostic assay can be any sample in which the cells targeted by the TBM may be present, from example a tumor (e.g., a solid tumor) biopsy, lymph, stool, urine, blood or any other bodily fluid that might contain circulating tumor cells.

### 6.12. Therapeutic Indications

The TBMs of the disclosure can be used in the treatment of any proliferative disorder (e.g., cancer) that expresses a TAA. In particular embodiments, the cancer is HER2+ cancer, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, or Wilms tumor.

Table 14 below shows exemplary indications that TBMs targeting particular TAAs can be used against.

TABLE 14
Examples of Tumor-Associated Antigen Indications

| Target | Exemplary Indication(s) |
|---|---|
| ADRB3 | Ewing sarcoma |
| ALK | NSCLC, ALCL, IMT, neuroblastoma |
| B7H3 | melanoma, osteosarcoma, leukemia, breast, prostate, ovarian, pancreatic, |
| | colorectal cancers |
| BCMA | multiple myeloma, leukemia (e.g., acute lymphoblastic leukemia (“ALL”), |
| | acute myeloid leukemia (“AML”), chronic lymphocytic leukemia (“CLL”), |
| | chronic myeloid leukemia (“CML”) and hairy cell leukemia (“HCL”)); |
| | lymphoma (e.g., Hodgkin’s lymphoma, non-Hodgkin’s lymphoma, |
| | including diffuse large B-cell lymphoma (“DLBCL”)) |
| Cadherin 17 | gastric, pancreatic, and colorectal adenocarcinomas |
| CAIX | clear-cell renal cell carcinoma, hypoxic solid tumors, head and neck |
| | squamous carcinoma |
| CD123 | leukemia (e.g., ALL, CLL, AML, CML, HCL); lymphoma (e.g., Hodgkin’s |
| | lymphoma, non-Hodgkin’s lymphoma, e.g., DLBCL); multiple myeloma. |
| | In a preferred embodiment, the indication is AML. |
| CD171 | neuroblastoma, paraganglioma |
| CD179a | B cell malignancies |
| CD19 | leukemia (e.g., ALL, CLL, AML, CML, HCL); lymphoma (e.g., Hodgkin’s |
| | lymphoma, non-Hodgkin's lymphoma, e.g., DLBCL); multiple myeloma. |
| CD20 | leukemia (e.g., ALL, CLL, AML, CML, HCL); lymphoma (e.g., Hodgkin’s |
| | lymphoma, non-Hodgkin's lymphoma, e.g., DLBCL); multiple myeloma. |
| CD22 | leukemia (e.g., ALL, CLL, AML, CML, HCL); lymphoma (e.g., Hodgkin’s |
| | lymphoma, non-Hodgkin’s lymphoma, e.g., DLBCL); multiple myeloma; |
| | lung cancer |
| CD24 | ovarian, breast, prostate, bladder, renal, non-small cell carcinomas |
| CD30 | anaplastic large cell lymphoma, embryonal carcinoma, Hodgkin Lymphoma |
| CD32b | B cell malignancies, gastric, pancreatic, esophageal, glioblastoma, breast, |
| | colorectal |
| CD33 | leukemia (e.g., ALL, CLL, AML, CML, HCL); lymphoma (e.g., Hodgkin’s |
| | lymphoma, non-Hodgkin’s lymphoma, e.g., DLBCL); multiple myeloma. In |
| | a preferred embodiment, the indication is AML. |
| CD38 | leukemia (e.g., ALL, CLL, AML, CML, HCL); lymphoma (e.g., Hodgkin’s |
| | lymphoma, non-Hodgkin’s lymphoma, e.g., DLBCL); multiple myeloma |
| CD44v6 | colon cancer, head and neck small cell carcinoma |
| CD97 | B cell malignancies, gastric, pancreatic, esophageal, glioblastoma, breast, |
| | colorectal |
| CEA | colorectal carcinoma, gastric carcinoma, pancreatic carcinoma, lung |
| | cancer, breast cancer, medullary thyroid carcinoma |
| CLDN6 | ovarian, breast, lung cancer |
| CLL-1 | leukemia (e.g., ALL, CLL, AML, CML, HCL); lymphoma (e.g., Hodgkin’s |
| | lymphoma, non-Hodgkin’s lymphoma, e.g., DLBCL); multiple myeloma. In |
| | a preferred embodiment, the indication is AML. |
| CS1 | multiple myeloma |
| EGFR | squamous cell carcinoma of lung, anal cancer, glioblastoma, epithelian |
| | tumors of head and neck, colon cancer |
| EGFRvIII | Glioblastoma |
| EPCAM | gastrointestestinal carcinoma, colorectal cancer |
| EphA2 | kaposi's sarcoma, glioblastoma, solid tumors, glioma |
| Ephrin B2 | thyroid cancer, breast cancer, malignant melanoma |
| ERBB2 | breast, ovarian, gastric cancers, lung adenocarcinoma, non-small cell lung |
| (Her2/neu) | cancer, uterine cancer, uterine serous endometrial carcinoma, salivary duct |
| | carcinoma, |
| FAP | pancreatic cancer, colorectal cancer, metastasis, epithelial cancers, soft |
| | tissue sarcomas |
| FCRL5 | multiple myeloma |
| FLT3 | leukemia (e.g., ALL, CLL, AML, CML, HCL); lymphoma (e.g., Hodgkin’s |
| | lymphoma, non-Hodgkin’s lymphoma, e.g., DLBCL); multiple myeloma. In |
| | a preferred embodiment, the indication is AML. |
| Folate receptor | ovarian, breast, renal, lung, colorectal, brain cancers |
| alpha | |
| Folate receptor | ovarian cancer |
| beta | |
| Fucosyl GM1 | AML, myeloma |
| GD2 | malignant melanoma, neuroblastoma |
| GD3 | Melanoma |
| GloboH | ovarian, gastric, prostate, lung, breast, and pancreatic cancers |
| gp100 | Melanoma |
| GPNMB | breast cancer, head and neck cancers |
| GPR20 | GIST |
| GPR64 | Ewing sarcoma, prostate, kidney and lung sarcomas |
| GPRC5D | multiple myeloma |
| HAVCR1 | renal cancer |
| HER3 | colon and gastric cancers |
| HMWMAA | melanoma, glioblastoma, breast cancer |
| IGF-I receptor | breast, prostate, lung cancers |
| IL-11Ra | papillary thyroid cancer, osteosarcoma, colorectal adenocarcinoma, |
| | lymphocytic leukemia |
| IL-13Ra2 | renal cell carcinoma, prostate cancer, gliomas, head and neck cancer, |
| | astrocytoma |
| KIT | myeloid leukemia, kaposi’s sarcoma, erythroleukemia, gastrointestinal |
| | stromal tumors |
| KLRG2 | breast cancers, lung cancers and ovarian cancers. |
| LewisY | squamous cell lung carcinoma, lung adenocarcinoma, ovarian carcinoma, |
| | and colorectal adenocarcinoma |
| LMP2 | prostate cancer, Hodgkin’s lymphoma, nasopharyngeal carcinoma |
| LRP6 | breast cancer |
| LY6K | breast, lung, ovarian, and cervical cancer |
| LYPD8 | colorectal and gastric cancers |
| Mesothelin | mesothelioma, pancreatic cancer, ovarian cancer, stomach cancer, lung |
| | cancer, endometrial cancer. |
| MUC1 | breast and ovarian cancers, lung, stomach, pancreatic, prostate cancers |
| NCAM | melanoma, Wilms’ tumor, small cell lung cancer, neuroblastoma, myeloma, |
| | paraganglioma, pancreatic acinar cell carcinoma, myeloid leukemia |
| NY-BR-1 | breast cancer |
| o-acetyl GD2 | neuroblastoma, melanoma |
| OR51E2 | prostate cancer |
| PANX3 | osteosarcoma |
| PLAC1 | hepatocellular carcinoma |
| Polysialic acid | small cell lung cancer |
| PDGFR-beta | myelomonocytic leukemia, chronic myeloid leukemia, acute myelogenous |
| | leukemia, acute lymphoblastic leukemia |
| PRSS21 | colon cancer, testicular cancer, ovarian cancer |
| PSCA | prostate cancer, gastric and bladder cancers |
| PSMA | prostate cancer, |
| ROR1 | metastatic cancers, chronic lymphocytic leukemia, solid tumors in lung, |
| | breast, ovarian, colon, pancreatic, sarcoma |
| SLC34A2 | bladder cancer |
| SLC39A6 | breast cancer, esophageal cancer |
| SLITRK6 | breast cancer, urothelial cancer, lung cancer |
| SSEA-4 | breast cancer, cancer stem cells, epithelial ovarian carcinoma |
| TACSTD2 | carcinomas, e.g., non-small-cell lung cancer |
| TAG72 | ovarian, breast, colon, lung, pancreatic cancers, gastric cancer |
| TEM1/CD248 | colorectal cancer |
| TEM7R | colorectal cancer |
| Tn | colorectal, breast cancers, cervical, lung, stomach cancers |
| TSHR | thyroid cancer, multiple myeloma |
| Tyrosinase | prostate cancer, melanoma |
| UPK2 | bladder cancer |
| VEGFR2 | ovarian and pancreatic cancers, renal cell carcinoma, colorectal cancer, |
| | medullary thyroid carcinoma |

Accordingly, the present disclosure provides methods of treating cancer comprising administering to a subject suffering from cancer a TBM in which ABM3 (i.e., the TAA ABM) binds to a TAA expressed on that type of cancer. In some embodiments, a TBM that targets a TAA identified in Table 14 is can be administered to a subject afflicted with a cancer that Table 14 indicates expressed the TAA. By way of example and not limitation, a TBM that targets EPCAM or folate receptor alpha can be administered to a subject afflicted with colorectal cancer, a TBM that targets BCMA or CD19 can be administered to a subject afflicted with a blood cancer such as multiple myeloma, a TBM that targets PSCA or PCMA can be administered a subject afflicted with prostate cancer, a TBM that targets tyrosinase or GP3 can be administered to a subject afflicted with melanoma, a TBM that targets CD33, CLL-1 or FLT3 can be administered to a subject afflicted with a blood cancer such as acute myeloid leukemia.

### 6.13. Combination Therapy

A TBM of the disclosure may be used in combination other known agents and therapies. For example, the TBMs of the disclosure can be used in treatment regimens in combination with surgery, chemotherapy, antibodies, radiation, peptide vaccines, steroids, cytoxins, or a combination thereof.

For convenience, an agent that is used in combination with a TBM of the disclosure is referred to herein as an “additional” agent.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. The term “concurrently” is not limited to the administration of therapies (e.g., a TBM and an additional agent) at exactly the same time, but rather it is meant that a pharmaceutical composition comprising a TBM of the disclosure is administered to a subject in a sequence and within a time interval such that the TBMs of the disclosure can act together with the additional therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic effect.

A TBM of the disclosure and one or more additional agents can be administered simultaneously, in the same or in separate compositions, or sequentially. For sequential administration, the TBM can be administered first, and the additional agent can be administered second, or the order of administration can be reversed.

The TBM and the additional agent(s) can be administered to a subject in any appropriate form and by any suitable route. In some embodiments, the routes of administration are the same. In other embodiments the routes of administration are different.

In other embodiments, the delivery of one treatment ends before the delivery of the other treatment begins.

In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The TBMs of the disclosure and/or additional agents can be administered during periods of active disorder, or during a period of remission or less active disease. A TBM can be administered before the treatment with the additional agent(s), concurrently with the treatment with the additional agent(s), post-treatment with the additional agent(s), or during remission of the disorder.

When administered in combination, the TBM and/or the additional agent(s) can be administered in an amount or dose that is higher, lower or the same than the amount or dosage of each agent used individually, e.g., as a monotherapy.

The additional agent(s) of the combination therapies of the disclosure can be administered to a subject concurrently. The term “concurrently” is not limited to the administration of therapies (e.g., prophylactic or therapeutic agents) at exactly the same time, but rather it is meant that a pharmaceutical composition comprising a TBM of the disclosure is administered to a subject in a sequence and within a time interval such that the molecules of the disclosure can act together with the additional therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route.

The TBM and the additional agent(s) may be administered to a subject by the same or different routes of administration.

The TBMs and the additional agent(s) may be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain instances, the one or more additional agents, are other anti-cancer agents, anti-allergic agents, anti-nausea agents (or anti-emetics), pain relievers, cytoprotective agents, and combinations thereof.

In one embodiment, a TBM of the disclosure can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab, obinutuzumab, ofatumumab, daratumumab, elotuzumab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with the TBMs of the present disclosure include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.04,9] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine- (SEQ ID NO: 718), inner salt (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

In certain aspects, “cocktails” of different chemotherapeutic agents are administered as the additional agent(s).

### 7. EXAMPLES

### 7.1. Example 1: Production and Characterization of Anti-CD19-Anti-CD3-Anti-CD2 Human IgG1 Bi- and Tri-Specific Antibodies

7.1.1. Materials and Methods

7.1.1.1. Gene Construction, Expression and Purification of Bi- and Tri-Specific Binding Molecules

Gene synthesis with mammalian expression codon optimization was performed externally for constructs represented in FIG. 2. Amino acid sequences for the constructs represented in FIG. 2 are shown in Table 15.

TABLE 15
Amino Acid Sequences of Bi- and Tri-specific Constructs

| | | |
|---|---|---|
| | | SEQ |
| Component | Sequence | ID NO: |

Bispecific Ab1
(CD19 scFV/hCD3 scFv 1×1 format)

| | | |
|---|---|---|
| Anti-CD19 scFv | | |
| HCDR1 (Kabat) | DYGVS | 689 |
| HCDR2 (Kabat) | VIWGSETTYYQSSLKS | 692 |
| HCDR3 (Kabat) | HYYYGGSYAMDY | 694 |
| HCDR1 (Chothia) | GVSLPDY | 719 |
| HCDR2 (Chothia) | WGSET | 720 |
| HCDR3 (Chothia) | HYYYGGSYAMDY | 694 |
| HCDR1 (IMGT) | GVSLPDY | 719 |
| HCDR2 (IMGT) | WGSET | 720 |
| HCDR3 (IMGT) | HYYYGGSYAMDY | 694 |
| VH | QVQLQESGPGLVKPSETLSLTCTVSGVSLPDYGVSWIRQ | 700 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SS | |
| LCDR1 (Kabat) | RASQDISKYLN | 695 |
| LCDR2 (Kabat) | HTSRLHS | 696 |
| LCDR3 (Kabat) | QQGNTLPYT | 697 |
| LCDR1 (Chothia) | SQDISKY | 721 |
| LCDR2 (Chothia) | HTS | 722 |
| LCDR3 (Chothia) | GNTLPY | 723 |
| LCDR1 (IMGT) | QDISKY | 724 |
| LCDR2 (IMGT) | HTS | 722 |
| LCDR3 (IMGT) | QQGNTLPYT | 697 |
| VL | EIVMTQSPATLSLSPGERATLSCRASQDISKYLNWYQQKP | 703 |
| | GQAPRLLIYHTSRLHSGIPARFSGSGSGTDYTLTISSLQPE | |
| | DFAVYFCQQGNTLPYTFGQGTKLEIK | |
| Linker | GGGGSGGGGSGGGGS | 1 |
| scFv (VH-Iinker-VL) | QVQLQESGPGLVKPSETLSLTCTVSGVSLPDYGVSWIRQ | 707 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SSGGGGSGGGGSGGGGSEIVMTQSPATLSLSPGERATL | |
| | SCRASQDISKYLNWYQQKPGQAPRLLIYHTSRLHSGIPAR | |
| | FSGSGSGTDYTLTISSLQPEDFAVYFCQQGNTLPYTFGQ | |
| | GTKLEIK | |
| Anti-CD3 scFv | | |
| HCDR1 (Kabat) | TYAMN | 131 |
| HCDR2 (Kabat) | RIRSKYNNYATYYADSVKD | 151 |
| HCDR3 (Kabat) | HGNFGNSYVSWFAY | 175 |
| HCDR1 (Chothia) | GFTFNTY | 199 |
| HCDR2 (Chothia) | RSKYNNYA | 212 |
| HCDR3 (Chothia) | HGNFGNSYVSWFAY | 175 |
| HCDR1 (IMGT) | GFTFNTYA | 725 |
| HCDR2 (IMGT) | IRSKYNNYAT | 726 |
| HCDR3 (IMGT) | VRHGNFGNSYVSWFAY | 182 |
| VH | EVQLVESGGGLVQPGGSLKLSCAASGFTFNTYAMNWVR | 114 |
| | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSS | |
| LCDR1 (Kabat) | RSSTGAVTTSNYAN | 132 |
| LCDR2 (Kabat) | GTNKRAP | 152 |
| LCDR3 (Kabat) | ALWYSNLWV | 176 |
| LCDR1 (Chothia) | STGAVTTSNY | 200 |
| LCDR2 (Chothia) | GTN | 213 |
| LCDR3 (Chothia) | WYSNLW | 223 |
| LCDR1 (IMGT) | TGAVTTSNY | 727 |
| LCDR2 (IMGT) | GTNKRAPWTP | 728 |
| LCDR3 (IMGT) | ALWYSNLWV | 176 |
| VL | QAWTQEPSLTVSPGGTVTLTCRSSTGAVTTSNYANWVQ | 115 |
| | QKPGQAPRGLIGGTNKRAPWTPARFSGSLLGDKAALTLS | |
| | GAQPEDEAEYFCALWYSNLWVFGGGTKLTVL | |
| Linker | GGGGSGGGGSGGGGSGGGGS | 48 |
| scFv (VH-linker-VL) | EVQLVESGGGLVQPGGSLKLSCAASGFTFNTYAMNWVR | 116 |
| | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSSGGGGSGGGGSGGGGSGGGGSQAVVTQ | |
| | EPSLTVSPGGTVTLTCRSSTGAVTTSNYANWVQQKPGQA | |
| | PRGLIGGTNKRAPWTPARFSGSLLGDKAALTLSGAQPED | |
| | EAEYFCALWYSNLWVFGGGTKLTVL | |
| Full Ab region | | |
| Linker CD19 arm | GGGGS | 25 |
| Fc-HC CD19 arm | DKTHTCPPCPAPELLGGPSVFLFPPKPKDTLMISRTPEVT | 729 |
| | CVVVAVSHEDPEVKFNWYVDGVEVHNAKTKPREEQYNS | |
| | TYRVVSVLTVLHQDWLNGKEYKCKVSNKALAAPIEKTISK | |
| | AKGQPREPQVYTLPPCRDELTKNQVSLWCLVKGFYPSDI | |
| | AVEWESNGQPENNYKTTPPVLDSDGSFFLYSKLTVDKSR | |
| | WQQGNVFSCSVMHEALHNHYTQKSLSLSPGK | |
| Full CD19 arm | QVQLQESGPGLVKPSETLSLTCTVSGVSLPDYGVSWIRQ | 730 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SSGGGGSGGGGSGGGGSEIVMTQSPATLSLSPGERATL | |
| | SCRASQDISKYLNWYQQKPGQAPRLLIYHTSRLHSGIPAR | |
| | FSGSGSGTDYTLTISSLQPEDFAVYFCQQGNTLPYTFGQ | |
| | GTKLEIKGGGGSDKTHTCPPCPAPELLGGPSVFLFPPKPK | |
| | DTLMISRTPEVTCVVVAVSHEDPEVKFNWYVDGVEVHNA | |
| | KTKPREEQYNSTYRVVSVLTVLHQDWLNGKEYKCKVSNK | |
| | ALAAPIEKTISKAKGQPREPQVYTLPPCRDELTKNQVSLW | |
| | CLVKGFYPSDIAVEWESNGQPENNYKTTPPVLDSDGSFF | |
| | LYSKLTVDKSRWQQGNVFSCSVMHEALHNHYTQKSLSLS | |
| | PGK | |
| Linker CD3 arm | GGGGS | 25 |
| Fc-HC CD3 arm | DKTHTCPPCPAPELLGGPSVFLFPPKPKDTLMISRTPEVT | 731 |
| | CVVVAVSHEDPEVKFNWYVDGVEVHNAKTKPREEQYNS | |
| | TYRVVSVLTVLHQDWLNGKEYKCKVSNKALAAPIEKTISK | |
| | AKGQPREPQVCTLPPSRDELTKNQVSLSCAVKGFYPSDI | |
| | AVEWESNGQPENNYKTTPPVLDSDGSFFLVSKLTVDKSR | |
| | WQQGNVFSCSVMHEALHNHYTQKSLSLSPGK | |
| Full CD3 arm | EVQLVESGGGLVQPGGSLKLSCAASGFTFNTYAMNWVR | 732 |
| | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSSGGGGSGGGGSGGGGSGGGGSQAVVTQ | |
| | EPSLTVSPGGTVTLTCRSSTGAVTTSNYANWVQQKPGQA | |
| | PRGLIGGTNKRAPWTPARFSGSLLGDKAALTLSGAQPED | |
| | EAEYFCALWYSNLWVFGGGTKLTVLGGGGSDKTHTCPP | |
| | CPAPELLGGPSVFLFPPKPKDTLMISRTPEVTCVVVAVSH | |
| | EDPEVKFNWYVDGVEVHNAKTKPREEQYNSTYRVVSVLT | |
| | VLHQDWLNGKEYKCKVSNKALAAPIEKTISKAKGQPREPQ | |
| | VCTLPPSRDELTKNQVSLSCAVKGFYPSDIAVEWESNGQ | |
| | PENNYKTTPPVLDSDGSFFLVSKLTVDKSRWQQGNVFSC | |
| | SVMHEALHNHYTQKSLSLSPGK | |

Bispecific Ab2
(hCD19 scFv/hCD58 ligand 1×1 format)

| | | |
|---|---|---|
| Anti-CD19 scFv | | |
| HCDR1 (Kabat) | DYGVS | 689 |
| HCDR2 (Kabat) | VIWGSETTYYQSSLKS | 692 |
| HCDR3 (Kabat) | HYYYGGSYAMDY | 694 |
| HCDR1 (Chothia) | GVSLPDY | 719 |
| HCDR2 (Chothia) | WGSET | 720 |
| HCDR3 (Chothia) | HYYYGGSYAMDY | 694 |
| HCDR1 (IMGT) | GVSLPDY | 719 |
| HCDR2 (IMGT) | WGSET | 720 |
| HCDR3 (IMGT) | HYYYGGSYAMDY | 694 |
| VH | QVQLQESGPGLVKPSETLSLTCTVSGVSLPDYGVSWIRQ | 700 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SS | |
| LCDR1 (Kabat) | RASQDISKYLN | 695 |
| LCDR2 (Kabat) | HTSRLHS | 696 |
| LCDR3 (Kabat) | QQGNTLPYT | 697 |
| LCDR1 (Chothia) | SQDISKY | 721 |
| LCDR2 (Chothia) | HTS | 722 |
| LCDR3 (Chothia) | GNTLPY | 723 |
| LCDR1 (IMGT) | QDISKY | 724 |
| LCDR2 (IMGT) | HTS | 722 |
| LCDR3 (IMGT) | QQGNTLPYT | 697 |
| VL | EIVMTQSPATLSLSPGERATLSCRASQDISKYLNWYQQKP | 703 |
| | GQAPRLLIYHTSRLHSGIPARFSGSGSGTDYTLTISSLQPE | |
| | DFAVYFCQQGNTLPYTFGQGTKLEIK | |
| Linker | GGGGSGGGGSGGGGS | 1 |
| scFv (VH-linker-VL) | QVQLQESGPGLVKPSETLSLTCTVSGVSLPDYGVSWIRQ | 707 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SSGGGGSGGGGSGGGGSEIVMTQSPATLSLSPGERATL | |
| | SCRASQDISKYLNWYQQKPGQAPRLLIYHTSRLHSGIPAR | |
| | FSGSGSGTDYTLTISSLQPEDFAVYFCQQGNTLPYTFGQ | |
| | GTKLEIK | |
| CD58 ligand | | |
| CD58 ligand | FSQQIYGVVYGNVTFHVPSNVPLKEVLWKKQKDKVAELE | 733 |
| | NSEFRAFSSFKNRVYLDTVSGSLTIYNLTSSDEDEYEMES | |
| | PNITDTMKFFLYVLESLPSPTLTCALTNGSIEVQCMIPEHY | |
| | NSHRGLIMYSWDCPMEQCKRNSTSIYFKMENDLPQKIQC | |
| | TLSNPLFNTTSSIILTTCIPSSGHSRHR | |
| Full Ab region | | |
| Linker CD19 arm | GGGGS | 25 |
| Fc-HC CD19 arm | DKTHTCPPCPAPELLGGPSVFLFPPKPKDTLMISRTPEVT | 729 |
| | CVVVAVSHEDPEVKFNWYVDGVEVHNAKTKPREEQYNS | |
| | TYRVVSVLTVLHQDWLNGKEYKCKVSNKALAAPIEKTISK | |
| | AKGQPREPQVYTLPPCRDELTKNQVSLWCLVKGFYPSDI | |
| | AVEWESNGQPENNYKTTPPVLDSDGSFFLYSKLTVDKSR | |
| | WQQGNVFSCSVMHEALHNHYTQKSLSLSPGK | |
| Full CD19 arm | QVQLQESGPGLVKPSETLSLTCTVSGVSLPDYGVSWIRQ | 730 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SSGGGGSGGGGSGGGGSEIVMTQSPATLSLSPGERATL | |
| | SCRASQDISKYLNWYQQKPGQAPRLLIYHTSRLHSGIPAR | |
| | FSGSGSGTDYTLTISSLQPEDFAVYFCQQGNTLPYTFGQ | |
| | GTKLEIKGGGGSDKTHTCPPCPAPELLGGPSVFLFPPKPK | |
| | DTLMISRTPEVTCVVVAVSHEDPEVKFNWYVDGVEVHNA | |
| | KTKPREEQYNSTYRVVSVLTVLHQDWLNGKEYKCKVSNK | |
| | ALAAPIEKTISKAKGQPREPQVYTLPPCRDELTKNQVSLW | |
| | CLVKGFYPSDIAVEWESNGQPENNYKTTPPVLDSDGSFF | |
| | LYSKLTVDKSRWQQGNVFSCSVMHEALHNHYTQKSLSLS | |
| | PGK | |
| Linker CD58 arm | GGGGS | 25 |
| Fc-HC CD3 arm | DKTHTCPPCPAPELLGGPSVFLFPPKPKDTLMISRTPEVT | 731 |
| | CVVVAVSHEDPEVKFNWYVDGVEVHNAKTKPREEQYNS | |
| | TYRVVSVLTVLHQDWLNGKEYKCKVSNKALAAPIEKTISK | |
| | AKGQPREPQVCTLPPSRDELTKNQVSLSCAVKGFYPSDI | |
| | AVEWESNGQPENNYKTTPPVLDSDGSFFLVSKLTVDKSR | |
| | WQQGNVFSCSVMHEALHNHYTQKSLSLSPGK | |
| Full CD58 arm | FSQQIYGVVYGNVTFHVPSNVPLKEVLWKKQKDKVAELE | 734 |
| | NSEFRAFSSFKNRVYLDTVSGSLTIYNLTSSDEDEYEMES | |
| | PNITDTMKFFLYVLESLPSPTLTCALTNGSIEVQCMIPEHY | |
| | NSHRGLIMYSWDCPMEQCKRNSTSIYFKMENDLPQKIQC | |
| | TLSNPLFNTTSSIILTTCIPSSGHSRHRGGGGSDKTHTCPP | |
| | CPAPELLGGPSVFLFPPKPKDTLMISRTPEVTCVVVAVSH | |
| | EDPEVKFNWYVDGVEVHNAKTKPREEQYNSTYRVVSVLT | |
| | VLHQDWLNGKEYKCKVSNKALAAPIEKTISKAKGQPREPQ | |
| | VCTLPPSRDELTKNQVSLSCAVKGFYPSDIAVEWESNGQ | |
| | PENNYKTTPPVLDSDGSFFLVSKLTVDKSRWQQGNVFSC | |
| | SVMHEALHNHYTQKSLSLSPGK | |

Trispecific Ab1
(CD19Fab/hCD3 scFv-CD58 ligand 1×1 format)

| | | |
|---|---|---|
| Anti-CD19 Fab | | |
| HCDR1 (Kabat) | DYGVS | 689 |
| HCDR2 (Kabat) | VIWGSETTYYQSSLKS | 692 |
| HCDR3 (Kabat) | HYYYGGSYAMDY | 694 |
| HCDR1 (Chothia) | GVSLPDY | 719 |
| HCDR2 (Chothia) | WGSET | 720 |
| HCDR3 (Chothia) | HYYYGGSYAMDY | 694 |
| HCDR1 (IMGT) | GVSLPDY | 719 |
| HCDR2 (IMGT) | WGSET | 720 |
| HCDR3 (IMGT) | HYYYGGSYAMDY | 694 |
| VH | QVQLQESGPGLVKPSETLSLTCTVSGVSLPDYGVSWIRQ | 700 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SS | |
| VH-CH1 Fab fragment | QVQLQESGPGLVKPSETLSLTCTVSGVSLPDYGVSWIRQ | 735 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSC | |
| LCDR1 (Kabat) | RASQDISKYLN | 695 |
| LCDR2 (Kabat) | HTSRLHS | 696 |
| LCDR3 (Kabat) | QQGNTLPYT | 697 |
| LCDR1 (Chothia) | SQDISKY | 721 |
| LCDR2 (Chothia) | HTS | 722 |
| LCDR3 (Chothia) | GNTLPY | 723 |
| LCDR1 (IMGT) | QDISKY | 724 |
| LCDR2 (IMGT) | HTS | 722 |
| LCDR3 (IMGT) | QQGNTLPYT | 697 |
| VL | EIVMTQSPATLSLSPGERATLSCRASQDISKYLNWYQQKP | 703 |
| | GQAPRLLIYHTSRLHSGIPARFSGSGSGTDYTLTISSLQPE | |
| | DFAVYFCQQGNTLPYTFGQGTKLEIK | |
| Light chain | EIVMTQSPATLSLSPGERATLSCRASQDISKYLNWYQQKP | 736 |
| | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| Anti-CD3 scFv | | |
| HCDR1 (Kabat) | TYAMN | 131 |
| HCDR2 (Kabat) | RIRSKYNNYATYYADSVKD | 151 |
| HCDR3 (Kabat) | HGNFGNSYVSWFAY | 175 |
| HCDR1 (Chothia) | GFTFNTY | 199 |
| HCDR2 (Chothia) | RSKYNNYA | 212 |
| HCDR3 (Chothia) | HGNFGNSYVSWFAY | 175 |
| HCDR1 (IMGT) | GFTFNTYA | 725 |
| HCDR2 (IMGT) | IRSKYNNYAT | 726 |
| HCDR3 (IMGT) | VRHGNFGNSYVSWFAY | 182 |
| VH | EVQLVESGGGLVQPGGSLKLSCAASGFTFNTYAMNWVR | 114 |
| | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSS | |
| LCDR1 (Kabat) | RSSTGAVTTSNYAN | 132 |
| LCDR2 (Kabat) | GTNKRAP | 152 |
| LCDR3 (Kabat) | ALWYSNLWV | 176 |
| LCDR1 (Chothia) | STGAVTTSNY | 200 |
| LCDR2 (Chothia) | GTN | 213 |
| LCDR3 (Chothia) | WYSNLW | 223 |
| LCDR1 (IMGT) | TGAVTTSNY | 727 |
| LCDR2 (IMGT) | GTNKRAPWTP | 728 |
| LCDR3 (IMGT) | ALWYSNLWV | 176 |
| VL | QAVVTQEPSLTVSPGGTVTLTCRSSTGAVTTSNYANWVQ | 115 |
| | QKPGQAPRGLIGGTNKRAPWTPARFSGSLLGDKAALTLS | |
| | GAQPEDEAEYFCALWYSNLWVFGGGTKLTVL | |
| Linker | GGGGSGGGGSGGGGSGGGGS | 48 |
| scFv (VH-linker-VL) | EVQLVESGGGLVQPGGSLKLSCAASGFTFNTYAMNWVR | 116 |
| | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSSGGGGSGGGGSGGGGSGGGGSQAVVTQ | |
| | EPSLTVSPGGTVTLTCRSSTGAVTTSNYANWVQQKPGQA | |
| | PRGLIGGTNKRAPWTPARFSGSLLGDKAALTLSGAQPED | |
| | EAEYFCALWYSNLWVFGGGTKLTVL | |
| CD58 ligand | | |
| CD58 ligand | FSQQIYGVVYGNVTFHVPSNVPLKEVLWKKQKDKVAELE | 733 |
| | NSEFRAFSSFKNRVYLDTVSGSLTIYNLTSSDEDEYEMES | |
| | PNITDTMKFFLYVLESLPSPTLTCALTNGSIEVQCMIPEHY | |
| | NSHRGLIMYSWDCPMEQCKRNSTSIYFKMENDLPQKIQC | |
| | TLSNPLFNTTSSIILTTCIPSSGHSRHR | |
| Full Ab region | | |
| Linker CD19-CD3 | GGGGSGGGGS | 47 |
| Linker CD3-Fc | GGGGS | 25 |
| Fc-HC CD19 arm | DKTHTCPPCPAPELLGGPSVFLFPPKPKDTLMISRTPEVT | 737 |
| | CVVVAVSHEDPEVKFNWYVDGVEVHNAKTKPREEQYGS | |
| | TYRVVSVLTVLHQDWLNGKEYKCKVSNKALAAPIEKTISK | |
| | AKGQPREPQVCTLPPSRDELTKNQVSLSCAVKGFYPSDI | |
| | AVEWESNGQPENNYKTTPPVLDSDGSFFLVSKLTVDKSR | |
| | WQQGNVFSCSVMHEALHNHYTQKSLSLSPGK | |
| Full CD19-CD3 arm | QVQLQESGPGLVKPSETLSLTCTVSGVSLPDYGVSWIRQ | 738 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCGGGGSGGGGSEV | |
| | QLVESGGGLVQPGGSLKLSCAASGFTFNTYAMNWVRQA | |
| | SGKGLEWVGRIRSKYNNYATYYADSVKDRFTISRDDSKS | |
| | TLYLQMNSLKTEDTAVYYCVRHGNFGNSYVSWFAYWGQ | |
| | GTLVTVSSGGGGSGGGGSGGGGSGGGGSQAVVTQEPS | |
| | LTVSPGGTVTLTCRSSTGAVTTSNYANWVQQKPGQAPR | |
| | GLIGGTNKRAPWTPARFSGSLLGDKAALTLSGAQPEDEA | |
| | EYFCALWYSNLWVFGGGTKLTVLGGGGSDKTHTCPPCP | |
| | APELLGGPSVFLFPPKPKDTLMISRTPEVTCVVVAVSHED | |
| | PEVKFNWYVDGVEVHNAKTKPREEQYGSTYRVVSVLTVL | |
| | HQDWLNGKEYKCKVSNKALAAPIEKTISKAKGQPREPQV | |
| | CTLPPSRDELTKNQVSLSCAVKGFYPSDIAVEWESNGQP | |
| | ENNYKTTPPVLDSDGSFFLVSKLTVDKSRWQQGNVFSCS | |
| | VMHEALHNHYTQKSLSLSPGK | |
| Linker CD58 ligand | GGGS | 739 |
| Fc CD58 ligand arm | DKTHTCPPCPAPELLGGPSVFLFPPKPKDTLMISRTPEVT | 740 |
| | CVVVDVSHEDPEVKFNWYVDGVEVHNAKTKPREEQYAS | |
| | TYRVVSVLTVLHQDWLNGKEYKCKVSNKALPAPIEKTISK | |
| | AKGQPREPQVYTLPPCREEMTKNQVSLWCLVKGFYPSDI | |
| | AVEWESNGQPENNYKTTPPVLDSDGSFFLYSKLTVDKSR | |
| | WQQGNVFSCSVMHEALHNRYTQKSLSLSPGK | |
| Full CD58 ligand arm | FSQQIYGVVYGNVTFHVPSNVPLKEVLWKKQKDKVAELE | 741 |
| | NSEFRAFSSFKNRVYLDTVSGSLTIYNLTSSDEDEYEMES | |
| | PNITDTMKFFLYVLESLPSPTLTCALTNGSIEVQCMIPEHY | |
| | NSHRGLIMYSWDCPMEQCKRNSTSIYFKMENDLPQKIQC | |
| | TLSNPLFNTTSSIILTTCIPSSGHSRHRGGGSDKTHTCPPC | |
| | PAPELLGGPSVFLFPPKPKDTLMISRTPEVTCVVVDVSHE | |
| | DPEVKFNWYVDGVEVHNAKTKPREEQYASTYRVVSVLTV | |
| | LHQDWLNGKEYKCKVSNKALPAPIEKTISKAKGQPREPQV | |
| | YTLPPCREEMTKNQVSLWCLVKGFYPSDIAVEWESNGQP | |
| | ENNYKTTPPVLDSDGSFFLYSKLTVDKSRWQQGNVFSCS | |
| | VMHEALHNRYTQKSLSLSPGK | |

Hetero-dimerization of Fc for the trispecific antibody was achieved by the ‘knobs-into-holes’ engineering (Ridgway et al. 1999, Protein Eng. 9(7):617-21). The extracellular domain of human CD58 was fused to human IgG1 Fc with a short flexible linker, while the other arm was encoded with a target binding Fab followed by a short flexible linker, followed by an anti-CD3 scFv, fused to human IgG1 Fc with a short flexible linker such as GGGGS (SEQ ID NO: 25). The corresponding full target light chain plasmid was also synthesized. Constant human IgG1 Fc sequence contained modifications that silence antibody dependent cellular cytotoxicity and facilitate purification of heterodimeric Fc multi-specific antibodies.

TBMs were expressed transiently in Human Embryonic Kidney (HEK293) cells. Briefly, transfection was performed using PEI (Polyethylenimine, MW 25,000 linear, Cat No. 23966-1, Polysciences, USA) as the transfection reagent. For small scale (≤1 L) transfections, cells were grown in flat-bottomed shake flasks on an orbital shaker (125 rpm) in a 37° C. humidified incubator at 8% CO2. Plasmids were combined with PEI at a final ratio of 1:3=DNA:PEI. 1 mg of plasmid per L culture was used for transfection at 2.0 million cells/mL media. After 5 days of expression, the antibody was harvested by clarification of the media via centrifugation and filtration. Purification was performed via Protein A affinity chromatography (rProtein A Sepharose Fast Flow, GE Healthcare Life Sciences, Uppsala, Sweden) by batch-binding using approximately 5 mL resin/L harvested supernatant. The batch-bound resin slurry was poured over Econo Pac® Chromatography Columns (Cat No. 7321010, BioRad Laboratories, Hercules, Calif.), and the resin bed washed with 10 CV of dPBS. Protein was eluted with 4 CV of 20 mM citrate, 125 mM NaCl, 50 mM sucrose, pH 3.0. The eluted protein was titrated to pH 5.5 with 1 M Sodium Citrate, pH 8.2. If the antibody contained aggregates, preparative size exclusion chromatography was performed using Superdex200 (GE Healthcare Life Sciences, Uppsala, Sweden) as a final polishing step with a mobile phase of 20 mM citrate, 125 mM NaCl, 50 mM sucrose, pH 5.5. Antibody titer, monomer content, and sample endotoxin levels were measured prior to assaying.

7.1.1.2. Re-Directed T Cell Cytotoxicity (Quantitative Luciferase Assay)

In a series of studies, a trispecific construct targeting human CD3, human CD2 and human CD19 was compared with a bispecific construct targeting human CD3 and human CD19 as well as a non-targeting control construct targeting human CD3 and human growth hormone (gH). The constructs were all analyzed for their potential to induce T cell-mediated apoptosis in tumor target cells. The amino acid sequences of the non-targeting control construct targeting human CD3 and gH are shown in Table 16.

TABLE 16
Amino Acid Sequences of anti-CD3 × anti-gH control antibody

| | | SEQ |
|---|---|---|
| Component | Sequence | ID NO: |
| anti-gH scFv-Fc | EVQLVESGGGLVKPGGSLRLSCAASGFTFSPYSVFWWVRQ | 742 |
| | APGKGLEWVSSINTDSTYKYYADSVKGRFTISRDNAENSI | |
| | FLQMNSLRAEDTAVYYCARDRSYYAFSSGSLSDYYYGLD | |
| | VWGQGTLVTVSSGGGGSGGGGSGGGGSGGGGSDIVMT | |
| | QSPLSLSVTPGEPASISCRSSQSLLHTNLYNYLDWYVQKP | |
| | GQSPQLLIYLASNRASGVPDRFSGSGSGTDFTLKISRVET | |
| | EDVGVYYCMQALQIPRTFGQGTKLEIKGGGGSDKTHTCP | |
| | PCPAPELLGGPSVFLFPPKPKDTLMISRTPEVTCVVVAVS | |
| | HEDPEVKFNWYVDGVEVHNAKTKPREEQYNSTYRVVSV | |
| | LTVLHQDWLNGKEYKCKVSNKALAAPIEKTISKAKGQPRE | |
| | PQVYTLPPCRDELTKNQVSLWCLVKGFYPSDIAVEWESN | |
| | GQPENNYKTTPPVLDSDGSFFLYSKLTVDKSRWQQGNVF | |
| | SCSVMHEALHNHYTQKSLSLSPGK | |
| anti-CD3 scFv-Fc | EVQLVESGGGLVQPGGSLKLSCAASGFTFNTYAMNWVR | 732 |
| | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSSGGGGSGGGGSGGGGSGGGGSQAVVTQ | |
| | EPSLTVSPGGTVTLTCRSSTGAVTTSNYANWVQQKPGQA | |
| | PRGLIGGTNKRAPWTPARFSGSLLGDKAALTLSGAQPED | |
| | EAEYFCALVVYSNLWVFGGGTKLTVLGGGGSDKTHTCPP | |
| | CPAPELLGGPSVFLFPPKPKDTLMISRTPEVTCVVVAVSH | |
| | EDPEVKFNWYVDGVEVHNAKTKPREEQYNSTYRVVSVLT | |
| | VLHQDWLNGKEYKCKVSNKALAAPIEKTISKAKGQPREPQ | |
| | VCTLPPSRDELTKNQVSLSCAVKGFYPSDIAVEWESNGQ | |
| | PENNYKTTPPVLDSDGSFFLVSKLTVDKSRWQQGNVFSC | |
| | SVMHEALHNHYTQKSLSLSPGK | |

The purified constructs were compared across multiple donor effector cells. Briefly, target cells (huCD19-expressing Nalm6 cells, huCD19-expressing Daudi cells, and human K562 cells (negative control)) were engineered to overexpress firefly luciferase. Cells were harvested and resuspended in RPMI medium (Invitrogen #11875-093) with 10% FBS. 10,000 target cells per well were plated in a flat-bottom 96-well plate. Human pan T effector cells were isolated via MACS negative selection (Miltenyi Biotec #130-096-535) from donors from cryopreserved PBMC (Cellular Technologies Limited #CTL-UP1; Hemacare #PB009C-1-AML) then added to the plate to obtain a final E:T ratio of 5:1 or 10:1. Co-cultured cells were incubated with a serial dilution of all constructs and controls. For normalization, average maximum luminescence refers to target cells co-incubated with effector cells, but without any test construct. After an incubation of either 24 or 48 hr at 37° C., 5% CO2, OneGlo luciferase substrate (Promega #E6120) was added to the plate. Luminescence was measured on an Envision plate reader after a 10 minute incubation. Percent specific lysis was calculated using the following equation:

Specific lysis (%)=(1−(sample luminescence/average maximum luminescence))*100

Studies were also performed in which 10,000 additional target cells were added to the co-cultures to mimic T cell exhaustion at 48 hr, 72 hr, and 96 hr with luciferase based cytotoxicity measurements being made at 48 hr, 72 hr, 96 hr and 120 hr. Additionally, the live cell populations at 48 and 96 hours were analyzed for by FACS for activation markers, checkpoint molecules, and phenotypic markers. Cells treated with 1 nM of the bispecific or trispecific construct were harvested via centrifugation after the first challenge at 48 hours or after the third challenge at 96 hours, and the live cell population was gated through either CD4 or CD8 and analyzed for the activation markers CD25 and CD69 (after the 96 hour challenge), checkpoint molecules PD1, LAG3, and TIM3 (after the 48 hour and 96 hour challenges), and the phenotypic markers CCR7 and CD45RO (after the 48 hour and 96 hour challenges).

7.1.1.3. Proliferation Assay

The purified TBM was tested by flow cytometry for its potential to induce proliferation of CD8+ or CD4+ T cells in the presence of human CD19-expressing Nalm6 tumor cells.

Briefly, freshly isolated human pan T cells were adjusted to 1 mio cells per ml in warm PBS and stained with 1 μM CellTrace Far Red (Invitrogen #C34564) at 37° C. for 20 minutes. The staining volume was quadrupled by addition of RPM11640 medium, containing 10% FBS. After incubation at 37° C. for an additional 5 min, the cells were washed once with pre-warmed medium to remove remaining dye. The stained effector T cells were adjusted to 1.111×10⁶ viable cells per ml in RPM11640 medium containing 10% FBS and BME. 180 μL of the cell suspension was added per well into a tissue culture treated 24-well plate. Nalm6 target cells were harvested, counted and checked for viability. Cells were adjusted to 0.222×10⁶ viable cells per ml in RPM11640 medium, containing 10% FBS and BME. 180 μL of the cell suspension was added to the plated effector T cells to obtain a final E:T ratio of 5:1. 40 μL of the test constructs were added to the cell-containing wells to obtain a final concentration of either 100, 10, 1 or 0.1 nM. As a positive control, effector T cells were plated in the absence of target cells or test construct with the addition of CD3/CD28 conjugated T-Activator DynaBeads (Invitrogen #111.41D). T cells plated alone with no stimuli served as a negative control.

After a four day incubation at 37° C., 5% CO2, cells were centrifuged (5 min, 300×g) and washed twice with 150 μL/well PBS, including 0.5% FBS.

Surface staining for CD8 (mouse IgGI.K; clone HIT8a; BD #555635) or CD4 (mouse IgGI.K; clone RPA-T4; BD #557695) was performed on ice for 1 hr, according to the supplier's suggestions. Cells were washed three times with 200 μL/well PBS containing 0.5% FBS, resuspended in 150 μL/well PBS with 0.5% FBS, and analyzed using a Beckman-Coulter FACS Cytoflex machine (CytExpert Software).

The live cell population was gated through either CD4 or CD8 for determination of subsequent proliferation of indicated cell type.

7.1.1.4. Cytokine Release Assay

The purified TBM targeting human CD3, human CD2 and human CD19 was analyzed for its ability to induce T cell-mediated de novo secretion of cytokines in the presence or absence of tumor target cells.

Briefly, huCD19-expressing Nalm6 target cells were harvested and resuspendend in RPMI medium with 10% FBS. 20 000 target cells per well were plated in a flat-bottom 96-well plate. Human pan T effector cells were isolated via MACS negative selection from cryopreserved PBMC then added to the plate to obtain a final E:T ratio of 5:1. Co-cultured cells were incubated with two concentrations of all constructs and controls. After an incubation of 24 hr at 37° C., 5% CO2 the supernatants were harvested by centrifugation at 300×g for 5 min for subsequent analysis.

A multiplexed ELISA was performed according to the manufacturer's instructions using a V-PLEX Proinflammatory Panel 1 Kit (MesoScale Discovery #K15049D).

7.1.1.5. Granzyme B ELISpot Assay

The purified TBM targeting human CD3, human CD2 and human CD19 was analyzed for its ability to induce Granzyme B secretion in the presence or absence of tumor target cells.

Briefly, Human pan T effector cells were isolated via MACS negative selection from cryopreserved PBMC. 50 000 effector cells per well were added to an ELISpot plate precoated with anti-Granzyme B antibody (MABTech #3485-4APW) and allowed to settle for 30 min at room temperature. 10 000 huCD19-expressing Nalm6 target cells were harvested and added carefully to the plate so as not to disturb the T cell layer. Co-cultured cells were incubated with two concentrations of all constructs and controls. After an incubation of 48 hr at 37° C., 5% CO2 the plate was washed and detection performed according to the manufacturer's instructions. The plate was dried and submitted for analysis to ZellNet consulting.

7.1.2. Results

7.1.2.1. Results: Re-Directed T Cell Cytotoxicity Assay

FIG. 3 shows that the TBM construct induced superior cytotoxicity in huCD19-expressing Nalm6 target cells compared to the bispecific construct in an assay performed with a final E:T ratio of 5:1 and either 24 or 48 hour incubation.

FIG. 4 shows that the TBM construct induced superior cytotoxic activity in huCD19-expressing Daudi target cells compared to the bispecific construct in an assay performed with a final E:T ratio of 5:1 and 48 hour incubation. As expected, the non-targeting anti-gH showed negligible cytotoxic activity.

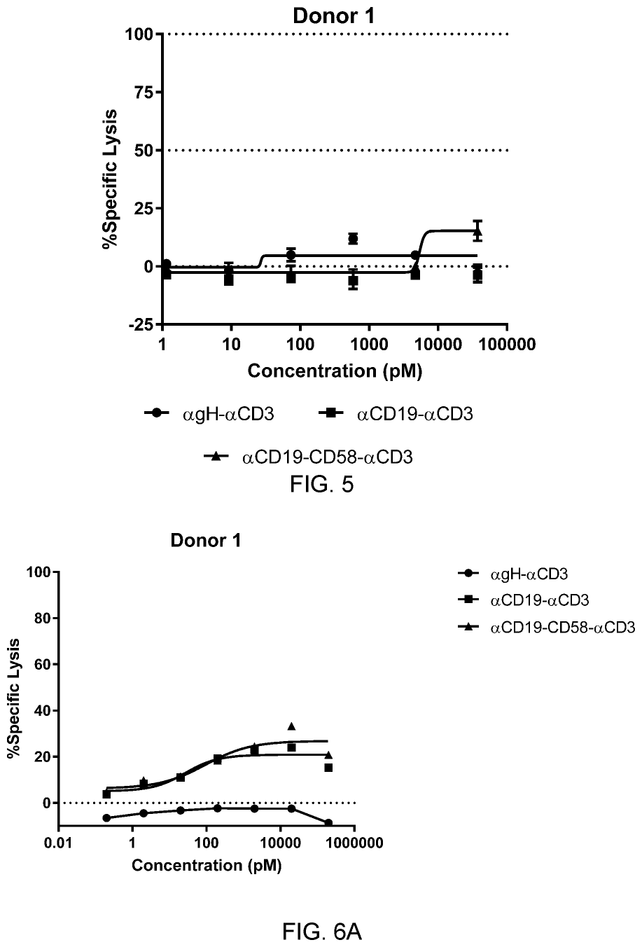

FIG. 5 shows that the bispecific and TBM constructs do not induce apoptosis of the negative control K562 cell line, which does not express human CD19, in an assay performed with a final E:T ratio of 5:1 and 48 hour incubation.

FIG. 6 shows that the TBM exhibited comparable or superior potency and efficacy compared to the bispecific construct in huCD19-expressing Nalm6 target cells when using human cryopreserved PBMCs that were thawed and rested overnight prior to use and with a final E:T ratio of 10:1 and 24 hour incubation.

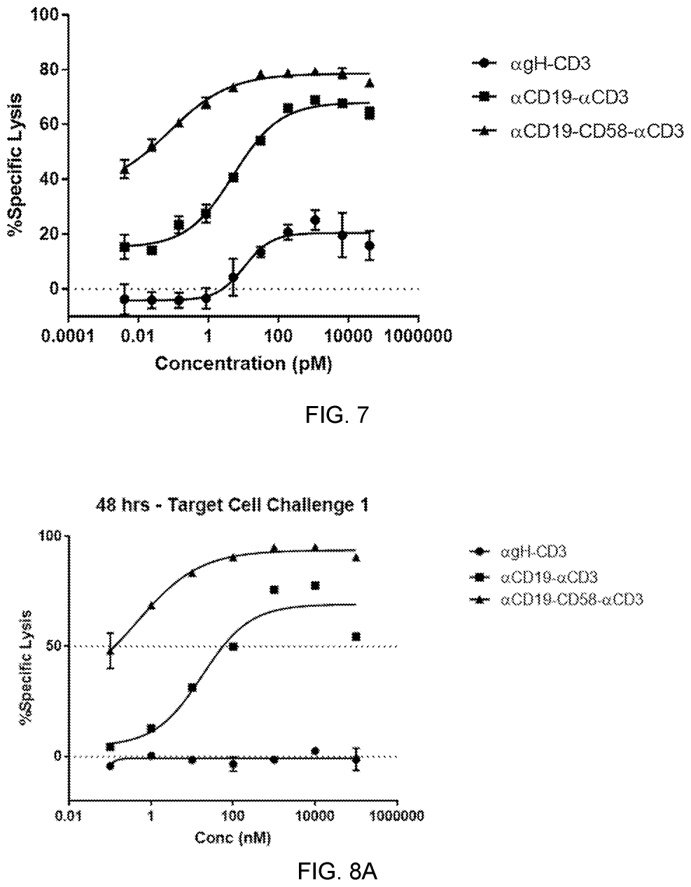

FIG. 7 shows that the TBM construct induced superior cytotoxic activity in huCD19-expressing Nalm6 target cells compared to the bispecific when using human cryopreserved PBMCs from a patient diagnosed with acute myeloid leukemia (Hemacare #PB009C-1-AML) that were thawed and rested overnight prior to use and with a final E:T ratio of 10:1 and 24 hour incubation. As expected, the non-targeting anti-gH showed negligible cytotoxic activity.

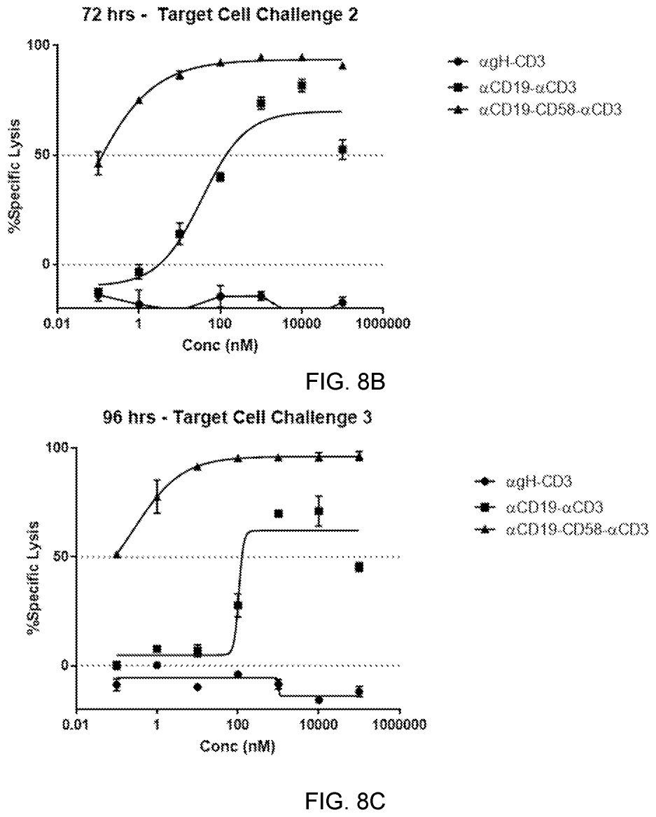

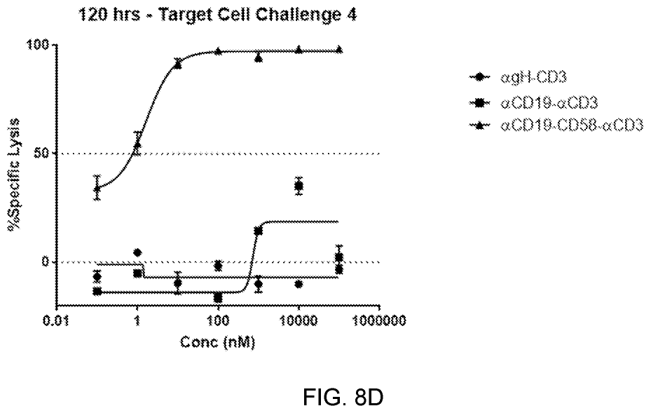

FIGS. 8A-8D shows that in a rechallenge assay with huCD19 expressing Nalm6 target cells, the TBM continued to induce apoptosis up to 120 hours with little apparent loss in activity across each rechallenge while the bispecific construct loses significant potency and activity with each subsequent target cell addition.

As expected, the non-targeting anti-gH showed negligible cytotoxic activity in each assay.

Table 17 shows the results of the FACS analysis for the activation markers CD25 and CD69 performed after the third challenge at 96 hours. The trispecific construct mediated higher levels of CD25 and double positive expression (CD25 and CD69) compared to the bispecific construct in both the CD4 and CD8 compartments. Values in Table 17 are expressed as percentage of positive cells.

TABLE 17

| | | | |
|---|---|---|---|
| Activation Marker | No Treatment | Bispecific at 1 nM | Trispecific at 1 nM |

CD4

| | | | |
|---|---|---|---|
| CD25+ | 4 | 3 | 54 |
| CD69+ | 2 | 5 | 10 |
| Double Positive | 0 | 0 | 19 |
| Double Negative | 94 | 92 | 17 |

CD8

| | | | |
|---|---|---|---|
| CD25+ | 0 | 7 | 78 |
| CD69+ | 2 | 13 | 4 |
| Double Positive | 0 | 4 | 6 |
| Double Negative | 98 | 76 | 12 |

Table 18 shows the results of the FACS analysis for the checkpoint molecules PD1, LAG3 and TIM3 performed after either the first challenge at 48 hours or the third challenge at 96 hours. The trispecific construct mediated higher levels of PD1, LAG3 and TIM3 compared to the bispecific construct in both the CD4 and CD8 compartments. Values in Table 18 are expressed as percentage of positive cells.

TABLE 18

| | Post Challenge 1 at 48 hours | | | Post Challenge 3 at 96 hours | | |
|---|---|---|---|---|---|---|
| Checkpoint | No | Bispecific | Trispecific | No | Bispecific | Trispecific |
| Marker | Treatment | at 1 nM | at 1 nM | Treatment | at 1 nM | at 1 nM |

CD4

| PD1+ | 7 | 12 | 25 | 7 | 5 | 40 |
|---|---|---|---|---|---|---|
| TIM3+ | 5 | 10 | 5 | 0 | 0 | 3 |
| Double | 0 | 2 | 17 | 0 | 0 | 26 |
| Positive | | | | | | |
| Double | 88 | 76 | 53 | 93 | 95 | 31 |
| Negative | | | | | | |
| Activation | No | Bispecific | Trispecific | No | Bispecific | Trispecific |
| Marker | Treatment | at 1 nM | at 1 nM | Treatment | at 1 nM | at 1 nM |

CD4

| PD1+ | 6 | 12 | 18 | 7 | 18 | 24 |
|---|---|---|---|---|---|---|
| LAG3+ | 0 | 0 | 1 | 0 | 0 | 4 |
| Double | 0 | 0 | 23 | 0 | 1 | 10 |
| Positive | | | | | | |
| Double | 94 | 88 | 58 | 93 | 81 | 62 |
| Negative | | | | | | |
| Checkpoint | No | Bispecific | Trispecific | No | Bispecific | Trispecific |
| Marker | Treatment | at 1 nM | at 1 nM | Treatment | at 1 nM | at 1 nM |

CD8

| PD1+ | 8 | 16 | 22 | 13 | 26 | 28 |
|---|---|---|---|---|---|---|
| TIM3+ | 6 | 9 | 8 | 0 | 0 | 12 |
| Double | 0 | 3 | 12 | 0 | 2 | 17 |
| Positive | | | | | | |
| Double | 86 | 72 | 58 | 87 | 72 | 43 |
| Negative | | | | | | |
| Activation | No | Bispecific | Trispecific | No | Bispecific | Trispecific |
| Marker | Treatment | at 1 nM | at 1 nM | Treatment | at 1 nM | at 1 nM |

CD8

| | | | | | | |
|---|---|---|---|---|---|---|
| PD1+ | 11 | 22 | 24 | 7 | 19 | 24 |
| LAG3+ | 1 | 1 | 1 | 0 | 0 | 4 |
| Double | 0 | 0 | 13 | 0 | 0 | 10 |
| Positive | | | | | | |
| Double | 88 | 77 | 62 | 93 | 81 | 62 |
| Negative | | | | | | |

Table 19 shows the results of the FACS analysis for the phenotypic markers CCR7 and CD45RO performed after the first challenge at 48 hours and the third challenge at 96 hours. The trispecific construct mediated a greater amount of central memory T cells compared to the bispecific construct. Values in Table 19 are expressed as percentage of positive cells.

TABLE 19

| | Post Challenge | | | Post Challenge | | |
|---|---|---|---|---|---|---|
| | 1 at 48 hours | | | 3 at 96 hours | | |
| | | Bi- | Tri- | | Bi- | Tri- |
| | | speci- | speci- | | speci- | speci- |
| | No | fic | fic | No | fic | fic |
| | Treat- | at 1 | at 1 | Treat- | at 1 | at 1 |
| Phenotype | ment | nM | nM | ment | nM | nM |
| CD4 | | | | | | |
| T_{effector} | 1 | 0 | 0 | 1 | 3 | 2 |
| (CCR7- | | | | | | |
| CD45RO−) | | | | | | |
| T_{effector memory} | 28 | 25 | 12 | 46 | 43 | 26 |
| (CCR7- | | | | | | |
| CD45RO+) | | | | | | |
| T_{central memory} | 38 | 47 | 61 | 25 | 18 | 62 |
| (CCR7+ | | | | | | |
| CD45RO+) | | | | | | |
| Tₙₐᵢᵥₑ | 33 | 28 | 27 | 28 | 36 | 10 |
| (CCR7+ | | | | | | |
| CD45RO−) | | | | | | |

7.1.2.2. Proliferation Assay

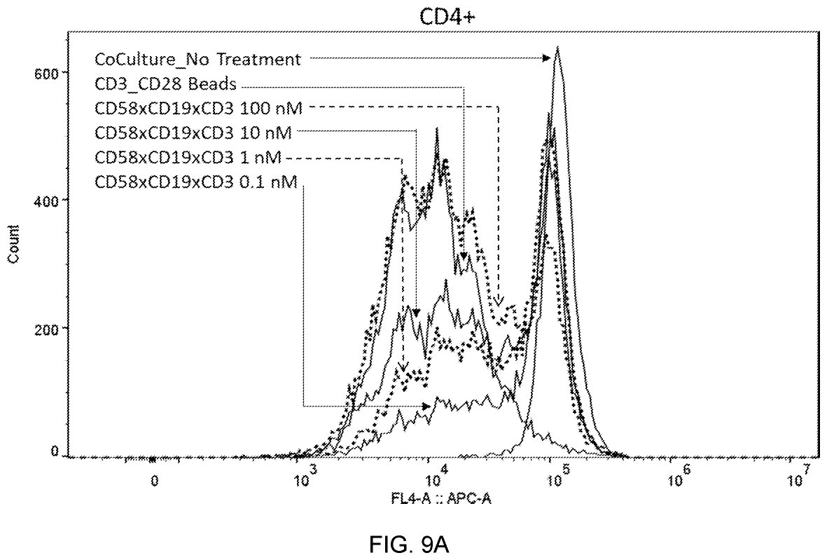

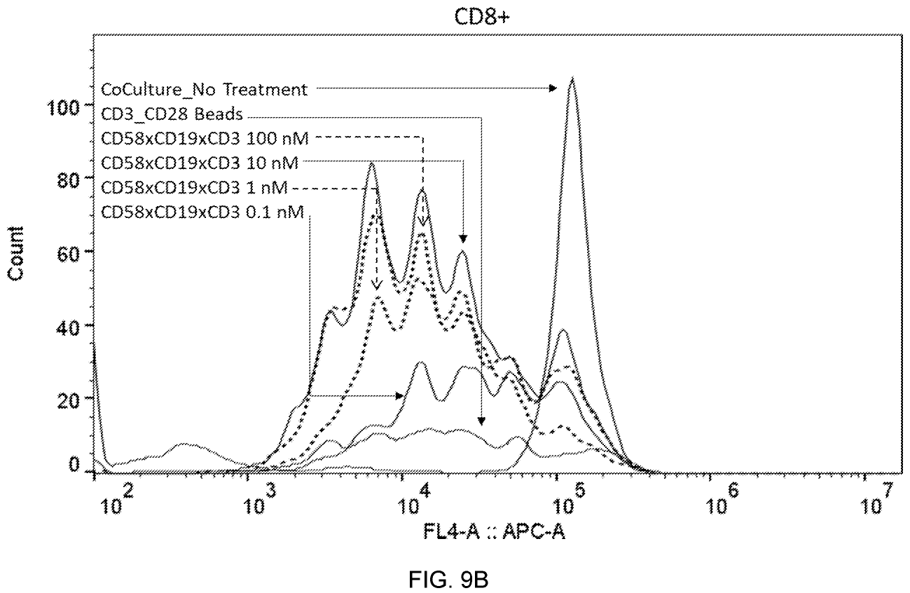

FIG. 9 shows that the TBM induces proliferation of CD4+ T cells or CD8+ T cells in the presence of target cells in a dose dependent manner. The TBM also appears to induce proliferation of CD8+ T cells to a greater extent than the CD3/CD28 conjugated T-Activator DynaBeads.

7.1.2.3. Cytokine Release Assay

FIG. 10 shows levels of the different cytokine measured in the supernatant. The results show that the trispecific molecule induced production of IFNγ, TNFα, IL2, IL6 and IL10 in human pan T effector cells in the presence of huCD19-expressing Nalm6 target cells. The induction by the CD2-CD3-CD19-targeting TBM was several fold greater than the CD3-CD19 bispecific antibody.

7.1.2.4. Results: Granzyme B ELISpot Assay

FIG. 11 shows that the number of Granzyme B spotforming T cells is much higher upon treatment with the CD2-CD3-CD19-targeting TBM than with the CD3-CD19 bispecific antibody.

### 7.2. Example 2: Anti-CD19-Anti-CD3-Anti-CD2 TBMs with Different CD2 Binding Arms

Trispecific binding molecules with different anti-human CD2 binding arms were generated and analyzed for their potential to induce T cell-mediated apoptosis in tumor target cells and for their potential to induce nuclear factor of activated T-cells (NFAT) in the absence of target cells.

7.2.1. Materials and Methods

7.2.1.1. Constructs

TBM constructs included the TBM of Example 1 having a full length CD58 moiety (AB2-1 in this Example), a TBM having a truncated CD58 comprising the IgV-like domain of CD58 (AB2-2 in this Example), and TBM having an scFv corresponding to the anti-CD2 antibody Medi 507 (AB2-3 in this Example). Amino acid sequences of the TBMs are shown in Table 20. The TBMs are schematically shown in FIG. 12.

TABLE 20
Amino Acid Sequences of TBMs of Example 2

| | | |
|---|---|---|
| | | SEQ |
| Component | Sequence | ID NO: |

AB2-1 (FIG. 12A)
(αCD19-CD58-αCD3)

| | | |
|---|---|---|
| αCD19 Light Chain | EIVMTQSPATLSLSPGERATLSCRASQDISKYLNWYQQKP | 736 |
| | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| CD58-Fc | FSQQIYGVVYGNVTFHVPSNVPLKEVLWKKQKDKVAELE | 741 |
| | NSEFRAFSSFKNRVYLDTVSGSLTIYNLTSSDEDEYEMES | |
| | PNITDTMKFFLYVLESLPSPTLTCALTNGSIEVQCMIPEHY | |
| | NSHRGLIMYSWDCPMEQCKRNSTSIYFKMENDLPQKIQC | |
| | TLSNPLFNTTSSIILTTCIPSSGHSRHRGGGSDKTHTCPPC | |
| | PAPELLGGPSVFLFPPKPKDTLMISRTPEVTCVVVDVSHE | |
| | DPEVKFNWYVDGVEVHNAKTKPREEQYASTYRVVSVLTV | |
| | LHQDWLNGKEYKCKVSNKALPAPIEKTISKAKGQPREPQV | |
| | YTLPPCREEMTKNQVSLWCLVKGFYPSDIAVEWESNGQP | |
| | ENNYKTTPPVLDSDGSFFLYSKLTVDKSRWQQGNVFSCS | |
| | VMHEALHNRYTQKSLSLSPGK | |
| αCD19_Fab- | QVQLQESGPGLVKPSETLSLTCTVSGVSLPDYGVSWIRQ | 738 |
| αCD3_scFv-Fc | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCGGGGSGGGGSEV | |
| | QLVESGGGLVQPGGSLKLSCAASGFTFNTYAMNWVRQA | |
| | SGKGLEWVGRIRSKYNNYATYYADSVKDRFTISRDDSKS | |
| | TLYLQMNSLKTEDTAVYYCVRHGNFGNSYVSWFAYWGQ | |
| | GTLVTVSSGGGGSGGGGSGGGGSGGGGSQAVVTQEPS | |
| | LTVSPGGTVTLTCRSSTGAVTTSNYANWVQQKPGQAPR | |
| | GLIGGTNKRAPWTPARFSGSLLGDKAALTLSGAQPEDEA | |
| | EYFCALVVYSNLWVFGGGTKLTVLGGGGSDKTHTCPPCP | |
| | APELLGGPSVFLFPPKPKDTLMISRTPEVTCVVVAVSHED | |
| | PEVKFNWYVDGVEVHNAKTKPREEQYGSTYRVVSVLTVL | |
| | HQDWLNGKEYKCKVSNKALAAPIEKTISKAKGQPREPQV | |
| | CTLPPSRDELTKNQVSLSCAVKGFYPSDIAVEWESNGQP | |
| | ENNYKTTPPVLDSDGSFFLVSKLTVDKSRWQQGNVFSCS | |
| | VMHEALHNHYTQKSLSLSPGK | |

AB2-2 (FIG. 12B)
(αCD19-CD58(IgV)-αCD3)

| | | |
|---|---|---|
| αCD19 Light Chain | EIVMTQSPATLSLSPGERATLSCRASQDISKYLNWYQQKP | 736 |
| | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| αCD19_Fab- | QVQLQESGPGLVKPSETLSLTCTVSGVSLPDYGVSWIRQ | 743 |
| αCD3_scFv-Fc | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCGGGGSGGGGSEV | |
| | QLVESGGGLVQPGGSLKLSCAASGFTFNTYAMNWVRQA | |
| | SGKGLEWVGRIRSKYNNYATYYADSVKDRFTISRDDSKS | |
| | TLYLQMNSLKTEDTAVYYCVRHGNFGNSYVSWFAYWGQ | |
| | GTLVTVSSGGGGSGGGGSGGGGSGGGGSQAVVTQEPS | |
| | LTVSPGGTVTLTCRSSTGAVTTSNYANWVQQKPGQAPR | |
| | GLIGGTNKRAPWTPARFSGSLLGDKAALTLSGAQPEDEA | |
| | EYFCALWYSNLWVFGGGTKLTVLGGGGSDKTHTCPPCP | |
| | APELLGGPSVFLFPPKPKDTLMISRTPEVTCVVVAVSHED | |
| | PEVKFNWYVDGVEVHNAKTKPREEQYASTYRVVSVLTVL | |
| | HQDWLNGKEYKCKVSNKALAAPIEKTISKAKGQPREPQV | |
| | CTLPPSREEMTKNQVSLSCAVKGFYPSDIAVEWESNGQP | |
| | ENNYKTTPPVLDSDGSFFLVSKLTVDKSRWQQGNVFSCS | |
| | VMHEALHNHYTQKSLSLSPGK | |
| CD58 IgV-Fc | SQQIYGVVYGNVTFHVPSNVPLKEVLWKKQKDKVAELEN | 744 |
| | SEFRAFSSFKNRVYLDTVSGSLTIYNLTSSDEDEYEMESP | |
| | NITDTMKFFLYVLESGGGGSDKTHTCPPCPAPELLGGPSV | |
| | FLFPPKPKDTLMISRTPEVTCVVVAVSHEDPEVKFNWYVD | |
| | GVEVHNAKTKPREEQYASTYRVVSVLTVLHQDWLNGKEY | |
| | KCKVSNKALAAPIEKTISKAKGQPREPQVYTLPPCREEMT | |
| | KNQVSLWCLVKGFYPSDIAVEWESNGQPENNYKTTPPVL | |
| | DSDGSFFLYSKLTVDKSRWQQGNVFSCSVMHEALHNRY | |
| | TQKSLSLSPGK | |

AB2-3 (FIG. 12C)
(αCD19-Medi507-αCD3)

| | | |
|---|---|---|
| αCD19 Light Chain | EIVMTQSPATLSLSPGERATLSCRASQDISKYLNWYQQKP | 736 |
| | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| αCD19_Fab- | QVQLQESGPGLVKPSETLSLTCTVSGVSLPDYGVSWIRQ | 743 |
| αCD3_scFv-Fc | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCGGGGSGGGGSEV | |
| | QLVESGGGLVQPGGSLKLSCAASGFTFNTYAMNWVRQA | |
| | SGKGLEWVGRIRSKYNNYATYYADSVKDRFTISRDDSKS | |
| | TLYLQMNSLKTEDTAVYYCVRHGNFGNSYVSWFAYWGQ | |
| | GTLVTVSSGGGGSGGGGSGGGGSGGGGSQAVVTQEPS | |
| | LTVSPGGTVTLTCRSSTGAVTTSNYANWVQQKPGQAPR | |
| | GLIGGTNKRAPWTPARFSGSLLGDKAALTLSGAQPEDEA | |
| | EYFCALWYSNLWVFGGGTKLTVLGGGGSDKTHTCPPCP | |
| | APELLGGPSVFLFPPKPKDTLMISRTPEVTCVVVAVSHED | |
| | PEVKFNWYVDGVEVHNAKTKPREEQYASTYRVVSVLTVL | |
| | HQDWLNGKEYKCKVSNKALAAPIEKTISKAKGQPREPQV | |
| | CTLPPSREEMTKNQVSLSCAVKGFYPSDIAVEWESNGQP | |
| | ENNYKTTPPVLDSDGSFFLVSKLTVDKSRWQQGNVFSCS | |
| | VMHEALHNHYTQKSLSLSPGK | |
| Medi 507 scFv-Fc | QVQLVQSGAEVQRPGASVKVSCKASGYIFTEYYMYWVR | 745 |
| | QAPGQGLELVGRIDPEDGSIDYVEKFKKKVTLTADTSSST | |
| | AYMELSSLTSDDTAVYYCARGKFNYRFAYWGQGTLVTVS | |
| | SGGGGSGGGGSGGGGSGGGGSDVVMTQSPPSLLVTLG | |
| | QPASISCRSSQSLLHSSGNTYLNWLLQRPGQSPQPLIYLV | |
| | SKLESGVPDRFSGSGSGTDFTLKISGVEAEDVGVYYCMQ | |
| | FTHYPYTFGQGTKLEIKGGGGSDKTHTCPPCPAPELLGG | |
| | PSVFLFPPKPKDTLMISRTPEVTCVVVAVSHEDPEVKFNW | |
| | YVDGVEVHNAKTKPREEQYNSTYRVVSVLTVLHQDWLN | |
| | GKEYKCKVSNKALAAPIEKTISKAKGQPREPQVCTLPPSR | |
| | DELTKNQVSLSCAVKGFYPSDIAVEWESNGQPENNYKTT | |
| | PPVLDSDGSFFLVSKLTVDKSRWQQGNVFSCSVMHEALH | |
| | NHYTQKSLSLSPGK | |

7.2.1.2. Re-Directed T Cell Cytotoxicity Assay

A re-directed T cell cytotoxicity assay was performed on the TBMs as described in Example 1, using human CD-19 expressing Nalm6 target cells at an E:T ratio of 5:1 and an incubation time of 48 hours.

A re-directed T cell cytotoxicity assay was also performed with AB2-2 using cynomolgus monkey pan T effector cells isolated via MACS negative selection (Miltenyi Biotec #130-091-993) from cryopreserved PBMC (iQ Biosciences #IQB-MnPB102). The cynomolgus monkey pan T effector cells were used in the assay at an E:T ratio of 5:1 and an incubation time of 48 hr.

7.2.1.3. NFAT Activation Assay

A Jurkat-NFAT reporter cell line can be used to evaluate the functional activity of trispecific constructs, specifically the non-specific activation of N FAT. Jurkat cells (cell line E6-1) stably expressing a NFAT-LUC reporter (JNL) were grown in RPMI-1640 media containing 2 mM glutamine and 10% fetal bovine serum with puromycin at 0.5 ug/ml. 100,000 JNL cells per well were plated in a flat-bottom 96-well plate and were incubated with a serial dilution of all constructs and controls. After an incubation of 6 hr at 37° C., 5% CO2, OneGlo luciferase substrate (Promega #E6120) was added to the plate. Luminescence was measured on an Envision plate reader after a 10 minute incubation.

7.2.2. Results

The results of the re-directed T cell cytotoxicity assay performed with Nalm6 target cells and human Pan T effector cells are shown in FIG. 13. The trispecific construct with the truncated CD58 IgV only domain (AB2-2) was observed to have similar cytotoxic activity to the trispecific construct having the full length CD58 molecule (AB2-1). The trispecific construct comprising a Medi 507 scFv (AB2-3) was observed in this assay to have superior cytotoxic activity compared to the trispecific constructs comprising a CD58 moiety.

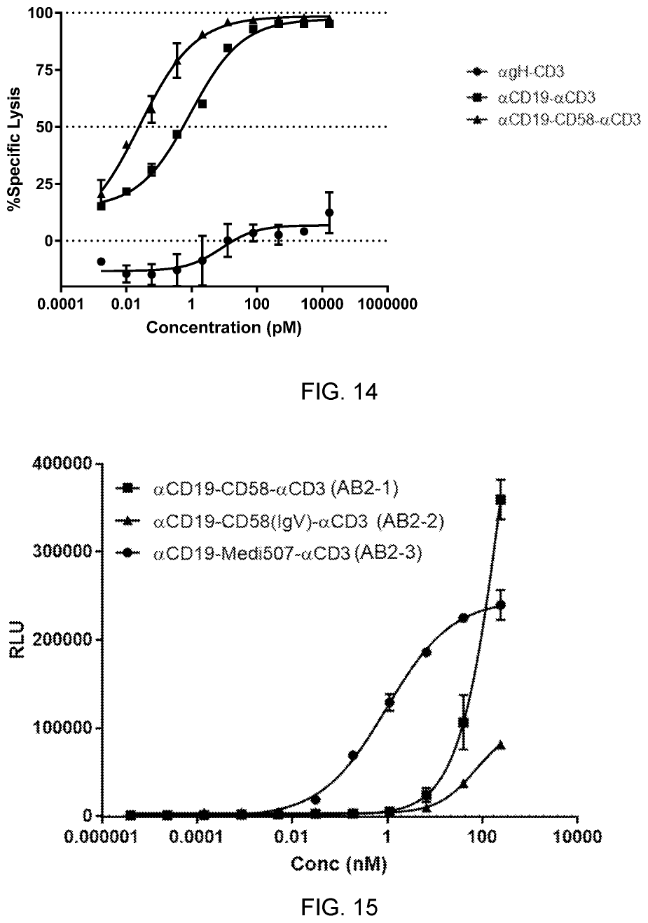

The results of the re-directed T cell cytotoxicity assay performed with Nalm6 target cells and cynomolgus monkey pan T effector cells are shown in FIG. 14. TBM AB2-2 exhibited superior cytotoxic activity in huCD19-expressing Nalm6 target cells compared to the bispecific molecule.

The results of the NFAT activation assay are shown in FIG. 15. The construct with truncated CD58 IgV only domain (AB2-2) was observed to induce less NFAT activation compared to the construct with the full length CD58 molecule (AB2-1). The construct having a scFv corresponding to the anti-CD2 antibody Medi 507 (AB2-3) showed a much greater induction of NFAT, indicating a higher potential for non-specific activation compared to the CD58 comprising trispecifics.

### 7.3. Example 3: Alternative Format Anti-CD19-Anti-CD3-Anti-CD2 TBMs

7.3.1. Materials and Methods

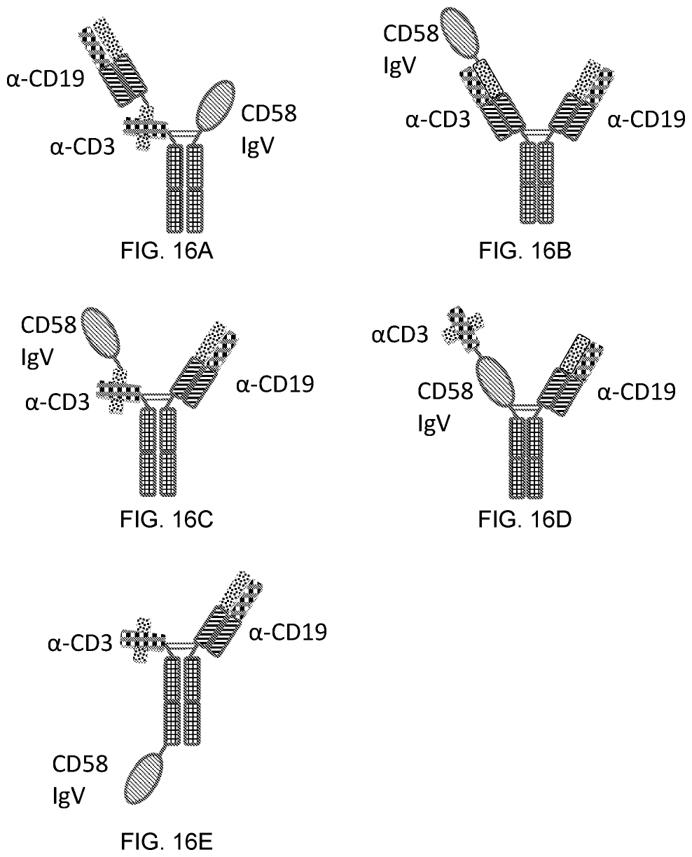

Trispecific binding molecules with various TBM formats were generated and analyzed for their potential to induce T cell-mediated apoptosis in tumor target cells. The TBM constructs included the TBM having the CD58 IgV domain from Example 2 (AB3-1 in this Example), a TBM having CD58 IgV domain N-terminal to an anti-CD3 scFab and an anti-CD19Fab (AB3-2), a TBM having CD58 IgV domain N-terminal to an anti-CD3 scFv, and an anti-CD19Fab (AB3-3), a TBM having an anti-CD3 scFv N-terminal to a CD58 IgV domain, and an anti-CD19 Fab (AB4-4), and a TBM having an anti-CD3 scFV, a C-terminal CD58 IgV domain, and an anti-CD19 Fab (AB4-5). Amino acid sequences of the TBMs of this Example are shown in Table 21. The TBMs are schematically shown in FIG. 16.

TABLE 21
Amino Acid Sequences of TBMs of Example 3

| | | |
|---|---|---|
| | | SEQ |
| Component | Sequence | ID NO: |

AB3-1 (FIG. 16A)
(αCD19-CD58(IgV)-αCD3)

| | | |
|---|---|---|
| Anti-CD19 Light | EIVMTQSPATLSLSPGERATLSCRASQDISKYLNWYQQKP | 736 |
| Chain | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| Anti-CD19 Fab-Anti- | QVQLQESGPGLVKPSETLSLTCTVSGVSLPDYGVSWIRQ | 738 |
| CD3_scFv-Fc | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCGGGGSGGGGSEV | |
| | QLVESGGGLVQPGGSLKLSCAASGFTFNTYAMNWVRQA | |
| | SGKGLEWVGRIRSKYNNYATYYADSVKDRFTISRDDSKS | |
| | TLYLQMNSLKTEDTAVYYCVRHGNFGNSYVSWFAYWGQ | |
| | GTLVTVSSGGGGSGGGGSGGGGSGGGGSQAVVTQEPS | |
| | LTVSPGGTVTLTCRSSTGAVTTSNYANWVQQKPGQAPR | |
| | GLIGGTNKRAPWTPARFSGSLLGDKAALTLSGAQPEDEA | |
| | EYFCALWYSNLWVFGGGTKLTVLGGGGSDKTHTCPPCP | |
| | APELLGGPSVFLFPPKPKDTLMISRTPEVTCVVVAVSHED | |
| | PEVKFNWYVDGVEVHNAKTKPREEQYGSTYRVVSVLTVL | |
| | HQDWLNGKEYKCKVSNKALAAPIEKTISKAKGQPREPQV | |
| | CTLPPSRDELTKNQVSLSCAVKGFYPSDIAVEWESNGQP | |
| | ENNYKTTPPVLDSDGSFFLVSKLTVDKSRWQQGNVFSCS | |
| | VMHEALHNHYTQKSLSLSPGK | |
| CD58 IgV-Fc | FSQQIYGVVYGNVTFHVPSNVPLKEVLWKKQKDKVAELE | 746 |
| | NSEFRAFSSFKNRVYLDTVSGSLTIYNLTSSDEDEYEMES | |
| | PNITDTMKFFLYVLESGGGGSDKTHTCPPCPAPELLGGPS | |
| | VFLFPPKPKDTLMISRTPEVTCVVVDVSHEDPEVKFNWYV | |
| | DGVEVHNAKTKPREEQYASTYRVVSVLTVLHQDWLNGKE | |
| | YKCKVSNKALPAPIEKTISKAKGQPREPQVYTLPPCREEM | |
| | TKNQVSLWCLVKGFYPSDIAVEWESNGQPENNYKTTPPV | |
| | LDSDGSFFLYSKLTVDKSRWQQGNVFSCSVMHEALHNR | |
| | YTQKSLSLSPGK | |

AB3-2 (FIG. 16B)
(CD58-αCD3Fab-αCD19Fab)

| | | |
|---|---|---|
| Anti-CD19 Heavy | QVQLQESGPGLVKPSETLSLTCTVSGVSLPDYGVSWIRQ | 747 |
| Chain | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCDKTHTCPPCPAPEL | |
| | LGGPSVFLFPPKPKDTLMISRTPEVTCVVVDVSHEDPEVK | |
| | FNWYVDGVEVHNAKTKPREEQYASTYRVVSVLTVLHQD | |
| | WLNGKEYKCKVSNKALPAPIEKTISKAKGQPREPQVCTLP | |
| | PSREEMTKNQVSLSCAVKGFYPSDIAVEWESNGQPENNY | |
| | KTTPPVLDSDGSFFLVSKLTVDKSRWQQGNVFSCSVMHE | |
| | ALHNHYTQKSLSLSPGK | |
| CD58-CD3_Fab-Fc | SQQIYGVVYGNVTFHVPSNVPLKEVLWKKQKDKVAELEN | 748 |
| | SEFRAFSSFKNRVYLDTVSGSLTIYNLTSSDEDEYEMESP | |
| | NITDTMKFFLYVLESLPSPTLTCALTNGSIEVQCMIPEHYN | |
| | SHRGLIMYSWDCPMEQCKRNSTSIYFKMENDLPQKIQCT | |
| | LSNPLFNTTSSIILTTCIPSSGHSRHRGGGGSGGGGSQAV | |
| | VTQEPSLTVSPGGTVTLTCRSSTGAVTTSNYANWVQQKP | |
| | GQAPRGLIGGTNKRAPWTPARFSGSLLGDKAALTLSGAQ | |
| | PEDEAEYFCALWYSNLWVFGGGTKLTVLGQPKAAPSVTL | |
| | FPPSSEELQANKATLVCLISDFYPGAVTVAWKADSSPVKA | |
| | GVETTTPSKQSNNKYAASSYLSLTPEQWKSHRSYSCQVT | |
| | HEGSTVEKTVAPTECSGSDKTHTCPPCPAPELLGGPSVF | |
| | LFPPKPKDTLMISRTPEVTCVVVDVSHEDPEVKFNWYVD | |
| | GVEVHNAKTKPREEQYASTYRVVSVLTVLHQDWLNGKEY | |
| | KCKVSNKALPAPIEKTISKAKGQPREPQVYTLPPCREEMT | |
| | KNQVSLWCLVKGFYPSDIAVEWESNGQPENNYKTTPPVL | |
| | DSDGSFFLYSKLTVDKSRWQQGNVFSCSVMHEALHNRY | |
| | TQKSLSLSPGK | |
| Anti-CD19 Light | EIVMTQSPATLSLSPGERATLSCRASQDISKYLNWYQQKP | 736 |
| Chain | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| Anti-CD3 Light Chain | EVQLVESGGGLVQPGGSLKLSCAASGFTFNTYAMNWVR | 749 |
| | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSSASTKGPSVFPLAPSSKSTSGGTAALGCLV | |
| | KDYFPEPVTVSWNSGALTSGVHTFPAVLQSSGLYSLSSV | |
| | VTVPSSSLGTQTYICNVNHKPSNTKVDKRVEPKSC | |

AB3-3 (FIG. 16C)
(CD58-αCD3scFv-αCD19Fab)

| | | |
|---|---|---|
| Anti-CD19 Light | EIVMTQSPATLSLSPGERATLSCRASQDISKYLNWYQQKP | 736 |
| Chain | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| Anti-CD19 Heavy | QVQLQESGPGLVKPSETLSLTCTVSGVSLPDYGVSWIRQ | 747 |
| Chain | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCDKTHTCPPCPAPEL | |
| | LGGPSVFLFPPKPKDTLMISRTPEVTCVVVDVSHEDPEVK | |
| | FNWYVDGVEVHNAKTKPREEQYASTYRVVSVLTVLHQD | |
| | WLNGKEYKCKVSNKALPAPIEKTISKAKGQPREPQVCTLP | |
| | PSREEMTKNQVSLSCAVKGFYPSDIAVEWESNGQPENNY | |
| | KTTPPVLDSDGSFFLVSKLTVDKSRWQQGNVFSCSVMHE | |
| | ALHNHYTQKSLSLSPGK | |
| CD58_AntiCD3scFv- | SQQIYGVVYGNVTFHVPSNVPLKEVLWKKQKDKVAELEN | 750 |
| Fc | SEFRAFSSFKNRVYLDTVSGSLTIYNLTSSDEDEYEMESP | |
| | NITDTMKFFLYVLESLPSPTLTCALTNGSIEVQCMIPEHYN | |
| | SHRGLIMYSWDCPMEQCKRNSTSIYFKMENDLPQKIQCT | |
| | LSNPLFNTTSSIILTTCIPSSGHSRHRGGGGSGGGGSEVQ | |
| | LVESGGGLVQPGGSLKLSCAASGFTFNTYAMNWVRQAS | |
| | GKGLEWVGRIRSKYNNYATYYADSVKDRFTISRDDSKSTL | |
| | YLQMNSLKTEDTAVYYCVRHGNFGNSYVSWFAYWGQGT | |
| | LVTVSSGGGGSGGGGSGGGGSGGGGSQAVVTQEPSLT | |
| | VSPGGTVTLTCRSSTGAVTTSNYANWVQQKPGQAPRGLI | |
| | GGTNKRAPWTPARFSGSLLGDKAALTLSGAQPEDEAEYF | |
| | CALWYSNLWVFGGGTKLTVLGGGGSDKTHTCPPCPAPE | |
| | LLGGPSVFLFPPKPKDTLMISRTPEVTCVVVDVSHEDPEV | |
| | KFNWYVDGVEVHNAKTKPREEQYASTYRVVSVLTVLHQD | |
| | WLNGKEYKCKVSNKALPAPIEKTISKAKGQPREPQVYTLP | |
| | PCREEMTKNQVSLWCLVKGFYPSDIAVEWESNGQPENN | |
| | YKTTPPVLDSDGSFFLYSKLTVDKSRWQQGNVFSCSVMH | |
| | EALHNRYTQKSLSLSPGK | |

AB3-4 (FIG. 16D)
(αCD3-CD58-αCD19Fab)

| | | |
|---|---|---|
| Anti-CD19 Light | EIVMTQSPATLSLSPGERATLSCRASQDISKYLNWYQQKP | 736 |
| Chain | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| Anti-CD19 Heavy | QVQLQESGPGLVKPSETLSLTCTVSGVSLPDYGVSWIRQ | 747 |
| Chain | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCDKTHTCPPCPAPEL | |
| | LGGPSVFLFPPKPKDTLMISRTPEVTCVVVDVSHEDPEVK | |
| | FNWYVDGVEVHNAKTKPREEQYASTYRVVSVLTVLHQD | |
| | WLNGKEYKCKVSNKALPAPIEKTISKAKGQPREPQVCTLP | |
| | PSREEMTKNQVSLSCAVKGFYPSDIAVEWESNGQPENNY | |
| | KTTPPVLDSDGSFFLVSKLTVDKSRWQQGNVFSCSVMHE | |
| | ALHNHYTQKSLSLSPGK | |
| AntiCD3scFv-CD58- | EVQLVESGGGLVQPGGSLKLSCAASGFTFNTYAMNWVR | 751 |
| Fc | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSSGGGGSGGGGSGGGGSGGGGSQAVVTQ | |
| | EPSLTVSPGGTVTLTCRSSTGAVTTSNYANWVQQKPGQA | |
| | PRGLIGGTNKRAPWTPARFSGSLLGDKAALTLSGAQPED | |
| | EAEYFCALWYSNLWVFGGGTKLTVLGGGGSGGGGSSQ | |
| | QIYGVVYGNVTFHVPSNVPLKEVLWKKQKDKVAELENSE | |
| | FRAFSSFKNRVYLDTVSGSLTIYNLTSSDEDEYEMESPNIT | |
| | DTMKFFLYVLESLPSPTLTCALTNGSIEVQCMIPEHYNSHR | |
| | GLIMYSWDCPMEQCKRNSTSIYFKMENDLPQKIQCTLSN | |
| | PLFNTTSSIILTTCIPSSGHSRHRGSDKTHTCPPCPAPELL | |
| | GGPSVFLFPPKPKDTLMISRTPEVTCVVVDVSHEDPEVKF | |
| | NWYVDGVEVHNAKTKPREEQYASTYRVVSVLTVLHQDW | |
| | LNGKEYKCKVSNKALPAPIEKTISKAKGQPREPQVYTLPP | |
| | CREEMTKNQVSLWCLVKGFYPSDIAVEWESNGQPENNY | |
| | KTTPPVLDSDGSFFLYSKLTVDKSRWQQGNVFSCSVMHE | |
| | ALHNRYTQKSLSLSPGK | |

AB3-5 (FIG. 16E)
(αCD3scFv-αCD19Fab-Fc-CD58)

| | | |
|---|---|---|
| Anti-CD19 Light | EIVMTQSPATLSLSPGERATLSCRASQDISKYLNWYQQKP | 736 |
| Chain | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| Anti-CD19 Heavy | QVQLQESGPGLVKPSETLSLTCTVSGVSLPDYGVSWIRQ | 747 |
| Chain | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCDKTHTCPPCPAPEL | |
| | LGGPSVFLFPPKPKDTLMISRTPEVTCVVVDVSHEDPEVK | |
| | FNWYVDGVEVHNAKTKPREEQYASTYRVVSVLTVLHQD | |
| | WLNGKEYKCKVSNKALPAPIEKTISKAKGQPREPQVCTLP | |
| | PSREEMTKNQVSLSCAVKGFYPSDIAVEWESNGQPENNY | |
| | KTTPPVLDSDGSFFLVSKLTVDKSRWQQGNVFSCSVMHE | |
| | ALHNHYTQKSLSLSPGK | |
| AntiCD3 scFv-Fc- | EVQLVESGGGLVQPGGSLKLSCAASGFTFNTYAMNWVR | 752 |
| CD58 | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSSGGGGSGGGGSGGGGSGGGGSQAVVTQ | |
| | EPSLTVSPGGTVTLTCRSSTGAVTTSNYANWVQQKPGQA | |
| | PRGLIGGTNKRAPWTPARFSGSLLGDKAALTLSGAQPED | |
| | EAEYFCALWYSNLWVFGGGTKLTVLGGGGSDKTHTCPP | |
| | CPAPELLGGPSVFLFPPKPKDTLMISRTPEVTCVVVDVSH | |
| | EDPEVKFNWYVDGVEVHNAKTKPREEQYASTYRVVSVLT | |
| | VLHQDWLNGKEYKCKVSNKALPAPIEKTISKAKGQPREPQ | |
| | VYTLPPCREEMTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGKGGGGSGGGGSGGGGS | |
| | GGGGSSQQIYGVVYGNVTFHVPSNVPLKEVLWKKQKDK | |
| | VAELENSEFRAFSSFKNRVYLDTVSGSLTIYNLTSSDEDE | |
| | YEMESPNITDTMKFFLYVLESLPSPTLTCALTNGSIEVQCM | |
| | IPEHYNSHRGLIMYSWDCPMEQCKRNSTSIYFKMENDLP | |
| | QKIQCTLSNPLFNTTSSIILTTCIPSSGHSRHR | |

Re-directed T cell cytotoxicity assays were performed on the TBMs as described in Example 1, using human CD-19 expressing Nalm6 target cells at an E:T ratio of 5:1 and an incubation time of 48 hours.

A NFAT activation assay to assess non-specific NFAT activation of the constructs was performed as described in Example 2.

7.3.2. Results

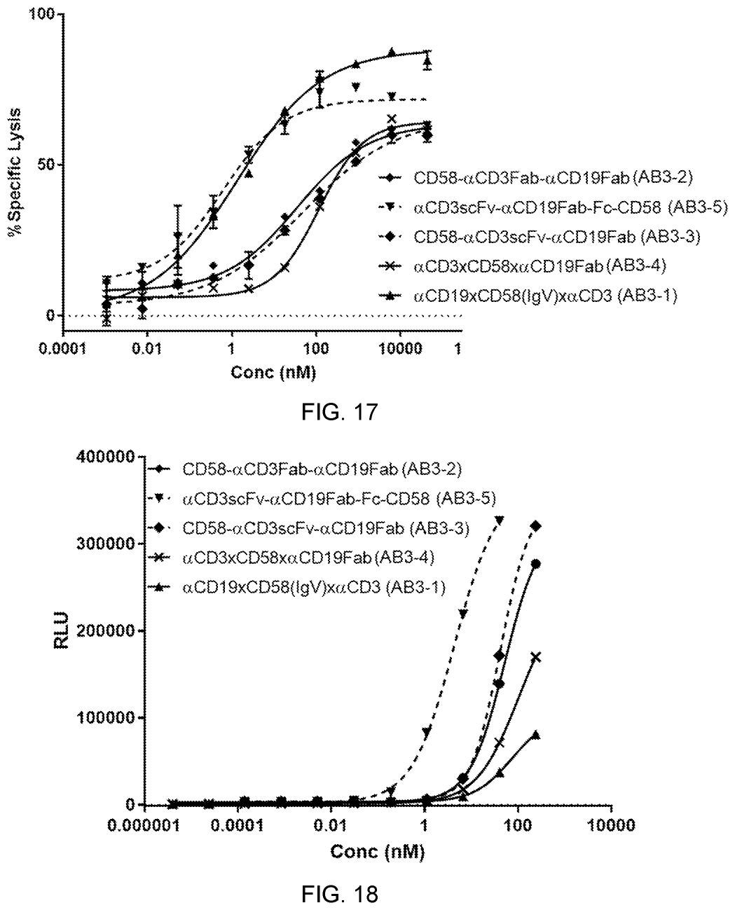

The results of the re-directed T cell cytotoxicity assay are shown in FIG. 17. The TBM with the truncated CD58 IgV only domain on the C-terminus (AB3-5) displayed similar cytotoxic activity to trispecific AB3-1. The other alternative formats showed inferior activity.

The results of the NFAT activation assay are shown in FIG. 18. The trispecific construct with the truncated CD58 IgV only domain on the C-terminus (AB3-5) displayed greater induction of NFAT compared to the other constructs. The original format (AB3-1) showed the lowest induction of NFAT.

### 7.4. Example 4: Anti-CD19-Anti-CD3-Anti-CD2 TBMs with Different CD19 Binding Arm Configurations

7.4.1. Materials and Methods

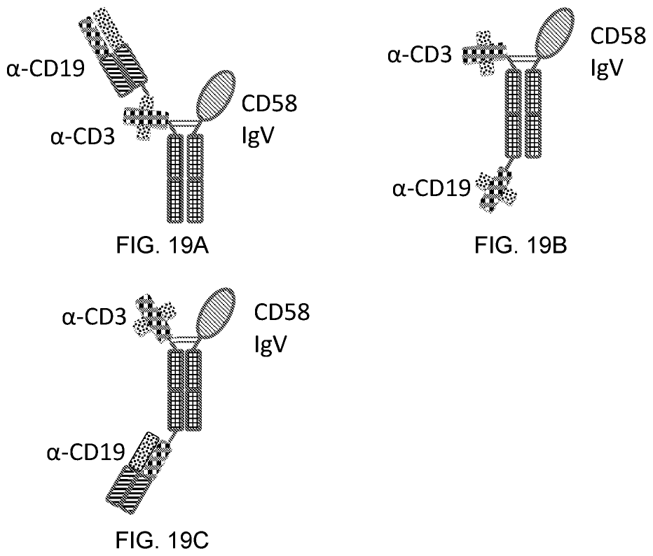

Trispecific binding molecules with different anti-CD19 arm configurations were analyzed for their potential to induce T cell-mediated apoptosis in tumor target cells. Constructs included the TBM having an anti-CD19 Fab N-terminal to an anti-CD3 scFV, and a CD58 IgV domain (the same construct as AB2-2 and AB3-1, labeled AB4-1 in this Example), a TBM having an anti-CD3scFv, a C-terminal anti-CD19 scFv, and a CD58 IgV domain (AB4-2), and a TBM having an anti-CD3scFv, a C-terminal anti-CD19 Fab, and a CD58 IgV domain (AB4-3). Amino acid sequences of the TBMs of this Example are shown in Table 22. The TBMs are schematically shown in FIG. 19.

TABLE 22
Amino Acid Sequences of TBMs of Example 4

| | | |
|---|---|---|
| | | SEQ |
| Component | Sequence | ID NO: |

AB4-1 (FIG. 19A)
(αCD19xCD58(IgV)xαCD3)

| | | |
|---|---|---|
| αCD19 Light Chain | EIVMTQSPATLSLSPGERATLSCRASQDISKYLNWYQQKP | 736 |
| | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| αCD19_Fab- | QVQLQESGPGLVKPSETLSLTCTVSGVSLPDYGVSWIRQ | 743 |
| αCD3_scFv-Fc | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCGGGGSGGGGSEV | |
| | QLVESGGGLVQPGGSLKLSCAASGFTFNTYAMNWVRQA | |
| | SGKGLEWVGRIRSKYNNYATYYADSVKDRFTISRDDSKS | |
| | TLYLQMNSLKTEDTAVYYCVRHGNFGNSYVSWFAYWGQ | |
| | GTLVTVSSGGGGSGGGGSGGGGSGGGGSQAVVTQEPS | |
| | LTVSPGGTVTLTCRSSTGAVTTSNYANWVQQKPGQAPR | |
| | GLIGGTNKRAPWTPARFSGSLLGDKAALTLSGAQPEDEA | |
| | EYFCALWYSNLWVFGGGTKLTVLGGGGSDKTHTCPPCP | |
| | APELLGGPSVFLFPPKPKDTLMISRTPEVTCVVVAVSHED | |
| | PEVKFNWYVDGVEVHNAKTKPREEQYASTYRVVSVLTVL | |
| | HQDWLNGKEYKCKVSNKALAAPIEKTISKAKGQPREPQV | |
| | CTLPPSREEMTKNQVSLSCAVKGFYPSDIAVEWESNGQP | |
| | ENNYKTTPPVLDSDGSFFLVSKLTVDKSRWQQGNVFSCS | |
| | VMHEALHNHYTQKSLSLSPGK | |
| CD58 IgV-Fc | SQQIYGVVYGNVTFHVPSNVPLKEVLWKKQKDKVAELEN | 744 |
| | SEFRAFSSFKNRVYLDTVSGSLTIYNLTSSDEDEYEMESP | |
| | NITDTMKFFLYVLESGGGGSDKTHTCPPCPAPELLGGPSV | |
| | FLFPPKPKDTLMISRTPEVTCVVVAVSHEDPEVKFNWWYVD | |
| | GVEVHNAKTKPREEQYASTYRVVSVLTVLHQDWLNGKEY | |
| | KCKVSNKALAAPIEKTISKAKGQPREPQVYTLPPCREEMT | |
| | KNQVSLWCLVKGFYPSDIAVEWESNGQPENNYKTTPPVL | |
| | DSDGSFFLYSKLTVDKSRWQQGNVFSCSVMHEALHNRY | |
| | TQKSLSLSPGK | |

AB4-2 (FIG. 19B)
(αCD3xCD58xαCD19scFv)

| | | |
|---|---|---|
| αCD3_scFv-Fc- | EVQLVESGGGLVQPGGSLKLSCAASGFTFNTYAMNWVR | 753 |
| αCD19_scFv | QASGKGLEWVGRIRSKYNNYATYYADSVKDRFTISRDDS | |
| | KSTLYLQMNSLKTEDTAWYCVRHGNFGNSYVSWFAYW | |
| | GQGTLVTVSSGGGGSGGGGSGGGGSGGGGSCAVVTQ | |
| | EPSLTVSPGGTVTLTCRSSTGAVTTSNYANWVQQKPGQA | |
| | PRGLIGGTNKRAPWTPARFSGSLLGDKAALTLSGAQPED | |
| | EAEYFCALWYSNLWVFGGGTKLTVLGGGGSDKTHTCPP | |
| | CPAPELLGGPSVFLFPPKPKDTLMISRTPEVTCVVVAVSH | |
| | EDPEVKFNWYVDGVEVHNAKTKPREEQYNSTYRVVSVLT | |
| | VLHQDWLNGKEYKCKVSNKALAAPIEKTISKAKGQPREPQ | |
| | VYTLPPCREEMTKNQVSLWQLVKGFYPSDAVEWESNGQ | |
| | PENNYKTTPPVLDSDGSFFLYSKLTVDKSRWQQGNVFSC | |
| | SVMHEALHNHYTQKSLSLSPGKGGGGSGGGGSGGGGS | |
| | GGGGSQVQLQESGPGLVKPSETLSLTCTVSGVSLPDYGV | |
| | SWIRQPPGKGLEWIGVIWGSETTYYQSSLKSRVTISKDNS | |
| | KNQVSLKLSSVTAADTAVYYCAKHYYYGGSYAMDYWGQ | |
| | GTLVTVSSGGGGSGGGGSGGGGSGGGGSEIVIVITQSPA | |
| | TLSLSPGERATLSCRASQDISKYLNWYQQKPGQAPRLLIY | |
| | HTSRLHSGIPARFSGSGSGTDYTLTISSLQPEDFAVYFCQ | |
| | QGNTLPYTFGQGTKLEIK | |
| CD58 IgV-Fc | SQQIYGVVYGNVTFHVPSNVPLKEVLWKKQKDKVAELEN | 754 |
| | SEFRAFSSFKNRVYLDTVSGSLTIYNLTSSDEDEYEMESP | |
| | NITDTMKFFLYVLESGGGSDKTHTCPPCPAPELLGGPSVF | |
| | LFPPKPKDTLMISRTPEVTCVVVAVSHEDPEVKFNWYVD | |
| | GVEVHNAKTKPREEQYGSTYRVVSVLTVLHQDWLNGKE | |
| | YKCKVSNKALAAPIEKTISKAKGQPREPQVCTLPPSRDEL | |
| | TKNQVSLSCAVKGFYPSDIAVEWESNGQPENNYKTTPPV | |
| | LDSDGSFFLVSKLTVDKSRWQQGNVFSCSVMHEALHNR | |
| | YTQKSLSLSPGK | |

AB4-3 (FIG. 19C)
(αCD3xCD58xαCD19Fab)

| | | |
|---|---|---|
| αCD19 Light Chain | EIVMTQSPATLSLSPGERATLSCRASQDISKYLNWYQQKP | 736 |
| | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| αCD3_scFv-Fc- | EVQLVESGGGLVQPGGSLKLSCAASGFTFNTYAMNWVR | 755 |
| αCD19_VH-CH1 | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSSGGGGSGGGGSGGGGSGGGGSQAVVTQ | |
| | EPSLTVSPGGTVTLTCRSSTGAVTTSNYANWVQQKPGQA | |
| | PRGLIGGTNKRAPWTPARFSGSLLGDKAALTLSGAQPED | |
| | EAEYFCALWYSNLWVFGGGTKLTVLGGGGSDKTHTCPP | |
| | CPAPELLGGPSVFLFPPKPKDTLMISRTPEVTCVVVAVSH | |
| | EDPEVKFNWYVDGVEVHNAKTKPREEQYNSTYRVVSVLT | |
| | VLHQDWLNGKEYKCKVSNKALAAPIEKTISKAKGQPREPQ | |
| | VYTLPPCREEMTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGKGGGGSGGGGSGGGGS | |
| | GGGGSQVQLQESGPGLVKPSETLSLTCTVSGVSLPDYGV | |
| | SWIRQPPGKGLEWIGVIWGSETTYYQSSLKSRVTISKDNS | |
| | KNQVSLKLSSVTAADTAVYYCAKHYYYGGSYAMDYWGQ | |
| | GTLVTVSSASTKGPSVFPLAPSSKSTSGGTAALGCLVKDY | |
| | FPEPVTVSWNSGALTSGVHTFPAVLQSSGLYSLSSVVTV | |
| | PSSSLGTQTYICNVNHKPSNTKVDKRVEPKSC | |
| CD58 IgV-Fc | SQQIYGVVYGNVTFHVPSNVPLKEVLWKKQKDKVAELEN | 754 |
| | SEFRAFSSFKNRVYLDTVSGSLTIYNLTSSDEDEYEMESP | |
| | NITDTMKFFLYVLESGGGSDKTHTCPPCPAPELLGGPSVF | |
| | LFPPKPKDTLMISRTPEVTCVVVAVSHEDPEVKFNWYVD | |
| | GVEVHNAKTKPREEQYGSTYRVVSVLTVLHQDWLNGKE | |
| | YKCKVSNKALAAPIEKTISKAKGQPREPQVCTLPPSRDEL | |
| | TKNQVSLSCAVKGFYPSDIAVEWESNGQPENNYKTTPPV | |
| | LDSDGSFFLVSKLTVDKSRWQQGNVFSCSVMHEALHNR | |
| | YTQKSLSLSPGK | |

Re-directed T cell cytotoxicity assays were performed on the TBMs as described in Example 1, using human CD-19 expressing Nalm6 target cells at an E:T ratio of 5:1 and an incubation time of 48 hours.

A NFAT activation assay to assess non-specific NFAT activation of the constructs was performed as described in Example 2.

A cytokine release assay as performed in Example 1 was performed using TBMs AB4-1, AB4-2, AB3-3, an anti-CD3-anti-CD19 bispecific construct, and an anti-gH-anti-CD3-CD58 trispecific construct.

7.4.2. Results

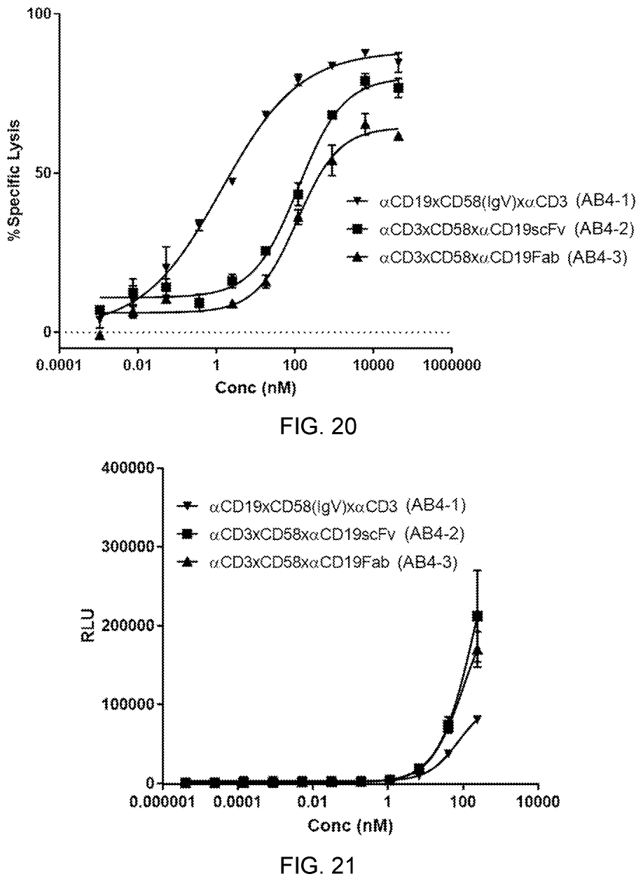

The results of the re-directed T cell cytotoxicity assay are shown in FIG. 20. TBMs with the CD19 binding arm on the C-terminus (AB4-2 and AB4-3) were observed to have less cytotoxic activity compared to the N-terminus format (AB4-1).

The results of the NFAT activation assay are shown in FIG. 21. Constructs with the CD19 binding arm on the C-terminus (AB4-2 and AB4-3) induced a higher induction of NFAT compared to the the N-terminus format (AB4-1).

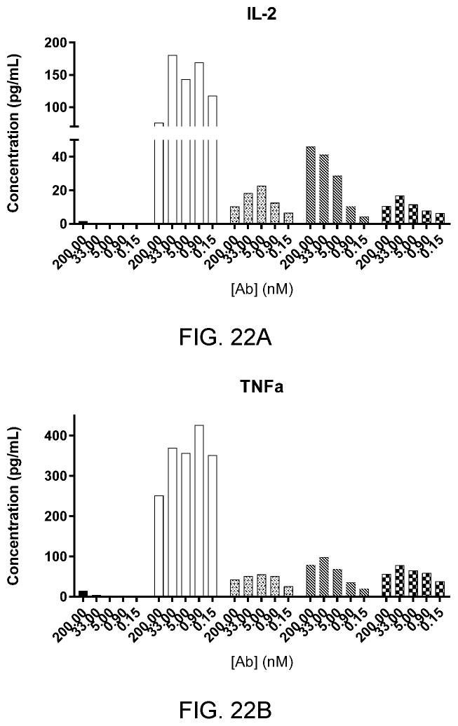

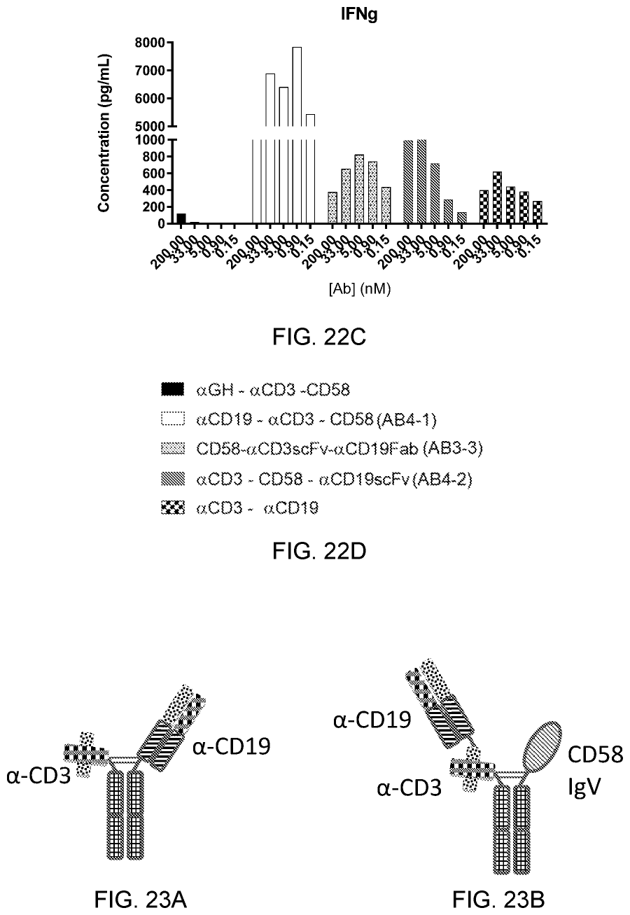

The results of the cytokine release assay are shown in FIG. 22. The AB4-1 construct format induced the highest cytokine levels. The other trispecific scaffolds tested stimulated cytokine levels that were relatively comparable to the bispecific format.

### 7.5. Example 5: Anti-CD19-Anti-CD3-Anti-CD2 TBMs with Different CD3 Binding Arms

7.5.1. Materials and Methods

Trispecific binding molecules with different anti-CD3 binding arms, namely scFvs corresponding to anti-CD3 antibodies BMA031 and OKT3, were generated and analyzed for their potential to induce T cell-mediated apoptosis in tumor target cells. The TBMs were compared to bispecific constructs targeting CD3 and CD19. Amino acid sequences of the bispecific and trispecific constructs used in this example are shown in Table 23. The bispecific and trispecfic constructs used in this Example are schematically shown in FIG. 23.

TABLE 23A
Amino Acid Sequences of BMA031 Constructs of Example 5

| | | |
|---|---|---|
| | | SEQ |
| Component | Sequence | ID NO: |

Bispecific (FIG. 23A)
αCD19-αCD3 (BMA031)

| | | |
|---|---|---|
| αCD19 Light Chain | EIVMTQSPATLSLSPGERATLSCRASQDISKYLNWYQQKP | 736 |
| | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| αCD19 Heavy Chain | QVQLQESGPGLVKPSETLSLTCTVSGVSLPDYGVSWIRQ | 756 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCDKTHTCPPCPAPEL | |
| | LGGPSVFLFPPKPKDTLMISRTPEVTCVVVAVSHEDPEVK | |
| | FNWYVDGVEVHNAKTKPREEQYASTYRVVSVLTVLHQD | |
| | WLNGKEYKCKVSNKALAAPIEKTISKAKGQPREPQVCTLP | |
| | PSREEMTKNQVSLSCAVKGFYPSDIAVEWESNGQPENNY | |
| | KTTPPVLDSDGSFFLVSKLTVDKSRWQQGNVFSCSVMHE | |
| | ALHNHYTQKSLSLSPGK | |
| BMA031 scFv-Fc | | |
| HCDR1 (Combined) | GYKFTSYVMH | 757 |
| HCDR2 (Combined) | YINPYNDVTKYNEKFKG | 758 |
| HCDR3 (Combined) | GSYYDYDGFVY | 243 |
| HCDR1 (Kabat) | SYVMH | 759 |
| HCDR2 (Kabat) | YINPYNDVTKYNEKFKG | 758 |
| HCDR3 (Kabat) | GSYYDYDGFVY | 243 |
| HCDR1 (Chothia) | GYKFTSY | 760 |
| HCDR2 (Chothia) | NPYNDV | 761 |
| HCDR3 (Chothia) | GSYYDYDGFVY | 243 |
| HCDR1 (IMGT) | GYKFTSYV | 762 |
| HCDR2 (IMGT) | INPYNDVT | 763 |
| HCDR3 (IMGT) | ARGSYYDYDGFVY | 764 |
| VH | EVQLQQSGPELVKPGASVKMSCKASGYKFTSYVMHWVK | 245 |
| | QKPGQGLEWIGYINPYNDVTKYNEKFKGKATLTSDKSSST | |
| | AYMELSSLTSEDSAVHYCARGSYYDYDGFVYWGQGTLV | |
| | TVSA | |
| LCDR1 (Combined) | SATSSVSYMH | 244 |
| LCDR2 (Combined) | DTSKLAS | 150 |
| LCDR3 (Combined) | QQWSSNPLT | 179 |
| LCDR1 (Kabat) | SATSSVSYMH | 244 |
| LCDR2 (Kabat) | DTSKLAS | 150 |
| LCDR3 (Kabat) | QQWSSNPLT | 179 |
| LCDR1 (Chothia) | TSSVSY | 765 |
| LCDR2 (Chothia) | DTS | 211 |
| LCDR3 (Chothia) | WSSNPL | 225 |
| LCDR1 (IMGT) | SSVSY | 766 |
| LCDR2 (IMGT) | DTS | 211 |
| LCDR3 (IMGT) | QQWSSNPLT | 179 |
| VL | QIVLTQSPAIMSASPGEKVTMTCSATSSVSYMHWYQQKS | 246 |
| | GTSPKRWIYDTSKLASGVPARFSGSGSGTSYSLTISSMEA | |
| | EDAATYYCQQWSSNPLTFGAGTKLELK | |
| BMA031 scFv-Fc full | EVQLQQSGPELVKPGASVKMSCKASGYKFTSYVMHWVK | 767 |
| sequence | QKPGQGLEWIGYINPYNDVTKYNEKFKGKATLTSDKSSST | |
| | AYMELSSLTSEDSAVHYCARGSYYDYDGFVYWGQGTLV | |
| | TVSAGGGGSGGGGSGGGGSGGGGSQIVLTQSPAIMSAS | |
| | PGEKVTMTCSATSSVSYMHWYQQKSGTSPKRWIYDTSK | |
| | LASGVPARFSGSGSGTSYSLTISSMEAEDAATYYCQQWS | |
| | SNPLTFGAGTKLELKGGGSDKTHTCPPCPAPELLGGPSV | |
| | FLFPPKPKDTLMISRTPEVTCVVVDVSHEDPEVKFNWYVD | |
| | GVEVHNAKTKPREEQYASTYRVVSVLTVLHQDWLNGKEY | |
| | KCKVSNKALPAPIEKTISKAKGQPREPQVYTLPPCREEMT | |
| | KNQVSLWCLVKGFYPSDIAVEWESNGQPENNYKTTPPVL | |
| | DSDGSFFLYSKLTVDKSRWQQGNVFSCSVMHEALHNHY | |
| | TQKSLSLSPGK | |

Trispecific (FIG. 23B)
αCD19-αCD3(BMA031)-CD58IgV

| | | |
|---|---|---|
| αCD19Light Chain | EIVMTQSPATLSLSPGERATLSCRASQDISKYLNWYQQKP | 736 |
| | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| αCD19_Fab- | QVQLQESGPGLVKPSETLSLTCTVSGVSLPDYGVSWIRQ | 768 |
| BMA031_scFv-Fc | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCGGGGSGGGGSEV | |
| | QLQQSGPELVKPGASVKMSCKASGYKFTSYVMHWVKQK | |
| | PGQGLEWIGYINPYNDVTKYNEKFKGKATLTSDKSSSTAY | |
| | MELSSLTSEDSAVHYCARGSYYDYDGFVYWGQGTLVTV | |
| | SAGGGGSGGGGSGGGGSGGGGSQIVLTQSPAIMSASPG | |
| | EKVTMTCSATSSVSYMHWYQQKSGTSPKRWIYDTSKLAS | |
| | GVPARFSGSGSGTSYSLTISSMEAEDAATYYCQQWSSNP | |
| | LTFGAGTKLELKGGGGSDKTHTCPPCPAPELLGGPSVFL | |
| | FPPKPKDTLMISRTPEVTCVVVAVSHEDPEVKFNWYVDG | |
| | VEVHNAKTKPREEQYGSTYRVVSVLTVLHQDWLNGKEYK | |
| | CKVSNKALAAPIEKTISKAKGQPREPQVCTLPPSRDELTK | |
| | NQVSLSCAVKGFYPSDIAVEWESNGQPENNYKTTPPVLD | |
| | SDGSFFLVSKLTVDKSRWQQGNVFSCSVMHEALHNHYT | |
| | QKSLSLSPGK | |
| CD58 IgV-Fc | SQQIYGVVYGNVTFHVPSNVPLKEVLWKKQKDKVAELEN | 744 |
| | SEFRAFSSFKNRVYLDTVSGSLTIYNLTSSDEDEYEMESP | |
| | NITDTMKFFLYVLESGGGGSDKTHTCPPCPAPELLGGPSV | |
| | FLFPPKPKDTLMISRTPEVTCVVVAVSHEDPEVKFNWYVD | |
| | GVEVHNAKTKPREEQYASTYRVVSVLTVLHQDWLNGKEY | |
| | KCKVSNKALAAPIEKTISKAKGQPREPQVYTLPPCREEMT | |
| | KNQVSLWCLVKGFYPSDIAVEWESNGQPENNYKTTPPVL | |
| | DSDGSFFLYSKLTVDKSRWQQGNVFSCSVMHEALHNRY | |
| | TQKSLSLSPGK | |

TABLE 23B
Amino Acid Sequences of OKT3 Constructs of Example 5

| | | |
|---|---|---|
| | | SEQ |
| Component | Sequence | ID NO: |

Bispecific (FIG. 23A)
αCD19-αCD3(OKT3)

| | | |
|---|---|---|
| αCD19Light Chain | EIVMTQSPATLSLSPGERATLSCRASQDISKYLNWYQQKP | 736 |
| | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| αCD19 Heavy Chain | QVQLQESGPGLVKPSETLSLTCTVSGVSLPDYGVSWIRQ | 756 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCDKTHTCPPCPAPEL | |
| | LGGPSVFLFPPKPKDTLMISRTPEVTCVVVAVSHEDPEVK | |
| | FNWYVDGVEVHNAKTKPREEQYASTYRVVSVLTVLHQD | |
| | WLNGKEYKCKVSNKALAAPIEKTISKAKGQPREPQVCTLP | |
| | PSREEMTKNQVSLSCAVKGFYPSDIAVEWESNGQPENNY | |
| | KTTPPVLDSDGSFFLVSKLTVDKSRWQQGNVFSCSVMHE | |
| | ALHNHYTQKSLSLSPGK | |
| OKT3 scFv-Fc | | |
| HCDR 1 (Combined) | GYTFTRYTMH | 231 |
| HCDR2 (Combined) | YINPSRGYTNYNQKVKD | 156 |
| HCDR3 (Combined) | YYDDHYSLDY | 188 |
| HCDR1 (Kabat) | RYTMH | 129 |
| HCDR2 (Kabat) | YINPSRGYTNYNQKVKD | 156 |
| HCDR3 (Kabat) | YYDDHYSLDY | 188 |
| HCDR1 (Chothia) | GYTFTRY | 197 |
| HCDR2 (Chothia) | NPSRGY | 210 |
| HCDR3 (Chothia) | YYDDHYSLDY | 188 |
| HCDR1 (IMGT) | GYTFTRYT | 769 |
| HCDR2 (IMGT) | INPSRGYT | 770 |
| HCDR3 (IMGT) | ARYYDDHYSLDY | 771 |
| VH | QVQLVQSGGGVVQPGRSLRLSCKASGYTFTRYTMHWVR | 111 |
| | QAPGKGLEWIGYINPSRGYTNYNQKVKDRFTISRDNSKNT | |
| | AFLQMDSLRPEDTGVYFCARYYDDHYSLDYWGQGTPVT | |
| | VSS | |
| LCDR1 (Combined) | SASSSVSYMN | 130 |
| LCDR2 (Combined) | DTSKLAS | 150 |
| LCDR3 (Combined) | QQWSSNPFT | 174 |
| LCDR1 (Kabat) | SASSSVSYMN | 130 |
| LCDR2 (Kabat) | DTSKLAS | 150 |
| LCDR3 (Kabat) | QQWSSNPFT | 174 |
| LCDR1 (Chothia) | SSSVSY | 198 |
| LCDR2 (Chothia) | DTS | 211 |
| LCDR3 (Chothia) | WSSNPF | 222 |
| LCDR1 (IMGT) | SSVSY | 766 |
| LCDR2 (IMGT) | DTS | 211 |
| LCDR3 (IMGT) | QQWSSNPFT | 174 |
| VL | DIQMTQSPSSLSASVGDRVTITCSASSSVSYMNWYQQTP | 87 |
| | GKAPKRWIYDTSKLASGVPSRFSGSGSGTDYTFTISSLQP | |
| | EDIATYYCQQWSSNPFTFGQGTKLQIT | |
| OKT3 scFv-Fc full | QVQLVQSGGGVVQPGRSLRLSCKASGYTFTRYTMHWVR | 772 |
| sequence | QAPGKGLEWIGYINPSRGYTNYNQKVKDRFTISRDNSKNT | |
| | AFLQMDSLRPEDTGVYFCARYYDDHYSLDYWGQGTPVT | |
| | VSSGGGGSGGGKSKKGGSGGGGSDIQMTQSPSSLSASV | |
| | GDRVTITCSASSSVSYMNWYQQTPGKAPKRWIYDTSKLA | |
| | SGVPSRFSGSGSGTDYTFTISSLQPEDIATYYCQQWSSN | |
| | PFTFGQGTKLQITGGGGSDKTHTCPPCPAPELLGGPSVF | |
| | LFPPKPKDTLMISRTPEVTCVVVAVSHEDPEVKFNWYVD | |
| | GVEVHNAKTKPREEQYASTYRVVSVLTVLHQDWLNGKEY | |
| | KCKVSNKALAAPIEKTISKAKGQPREPQVYTLPPCREEMT | |
| | KNQVSLWCLVKGFYPSDIAVEWESNGQPENNYKTTPPVL | |
| | DSDGSFFLYSKLTVDKSRWQQGNVFSCSVMHEALHNHY | |
| | TQKSLSLSPGK | |

Trispecific (FIG. 23B)
αCD19-αCD3(OKT3)-CD58IgV

| | | |
|---|---|---|
| αCD19Light Chain | EIVMTQSPATLSLSPGERATLSCRASQDISKYLNWYQQKP | 736 |
| | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| αCD19_Fab- | QVQLQESGPGLVKPSETLSLTCTVSGVSLPDYGVSWIRQ | 773 |
| OKT3_scFv-Fc | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCGGGGSGGGGSQV | |
| | QLVQSGGGVVQPGRSLRLSCKASGYTFTRYTMHWVRQA | |
| | PGKGLEWIGYINPSRGYTNYNQKVKDRFTISRDNSKNTAF | |
| | LQMDSLRPEDTGVYFCARYYDDHYSLDYWGQGTPVTVS | |
| | SGGGGSGGGGSGGGGSGGGGSDIQMTQSPSSLSASVG | |
| | DRVTITCSASSSVSYMNWYQQTPGKAPKRWIYDTSKLAS | |
| | GVPSRFSGSGSGTDYTFTISSLQPEDIATYYCQQWSSNP | |
| | FTFGQGTKLQITGGGGSDKTHTCPPCPAPELLGGPSVFLF | |
| | PPKPKDTLMISRTPEVTCVVVAVSHEDPEVKFNWYVDGV | |
| | EVHNAKTKPREEQYG | |
| | STYRVVSVLTVLHQDWLNGKEYKCKVSNKALAAPIEKTIS | |
| | KAKGQPREPQVCTLPPSRDELTKNQVSLSCAVKGFYPSD | |
| | IAVEWESNGQPENNYKTTPPVLDSDGSFFLVSKLTVDKSR | |
| | WQQGNVFSCSVMHEALHNHYTQKSLSLSPGK | |
| CD58 IgV-Fc | SQQIYGVVYGNVTFHVPSNVPLKEVLWKKQKDKVAELEN | 744 |
| | SEFRAFSSFKNRVYLDTVSGSLTIYNLTSSDEDEYEMESP | |
| | NITDTMKFFLYVLESGGGGSDKTHTCPPCPAPELLGGPSV | |
| | FLFPPKPKDTLMISRTPEVTCVVVAVSHEDPEVKFNEWYVD | |
| | GVEVHNAKTKPREEQYASTYRVVSVLTVLHQDWLNGKEY | |
| | KCKVSNKALAAPIEKTISKAKGQPREPQVYTLPPCREEMT | |
| | KNQVSLWCLVKGFYPSDIAVEWESNGQPENNYKTTPPVL | |
| | DSDGSFFLYSKLTVDKSRWQQGNVFSCSVMHEALHNRY | |
| | TQKSLSLSPGK | |

Re-directed T cell cytotoxicity assays were performed using the constructs as described in Example 1, using human CD-19 expressing Nalm6 target cells at an E:T ratio of 5:1 and an incubation time of 48 hours.

Cytokine release assays were also performed as in Example 1.

7.5.2. Results

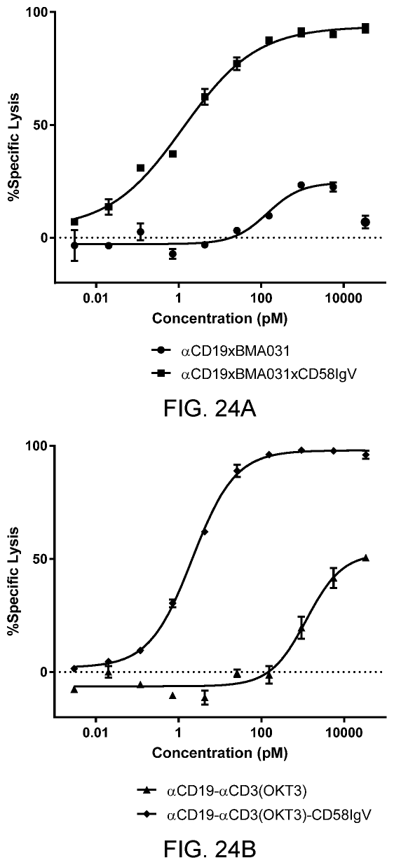

The results of the re-directed T cell cytotoxicity assay are shown in FIG. 24A and FIG. 24B. The two trispecfic constructs exhibited superior cytotoxicity in target cells compared to the bispecific constructs.

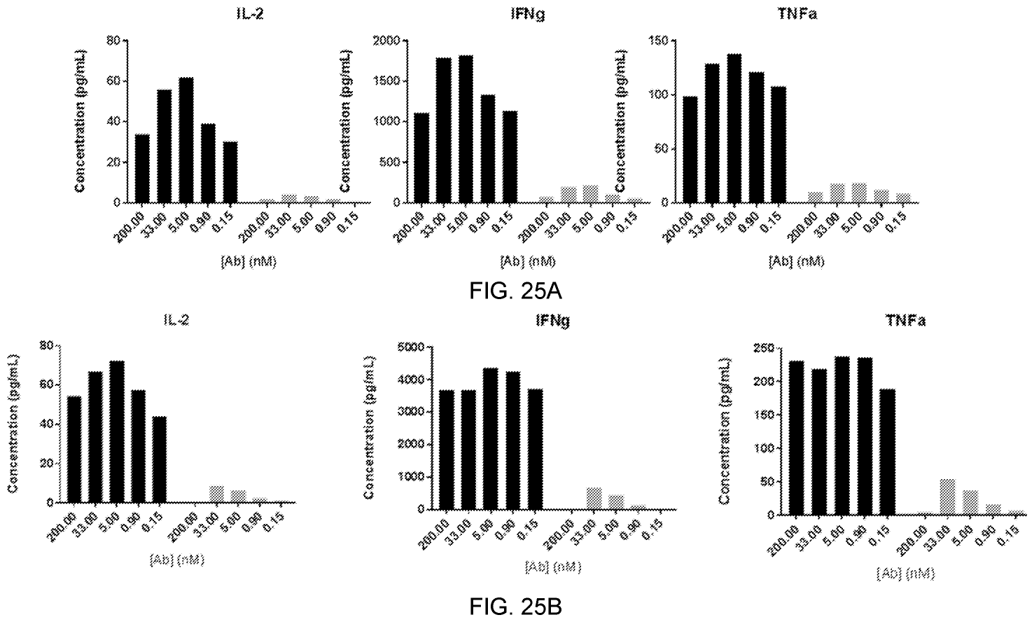

Results of the cytokine release assay are shown in FIG. 25. The trispecific constructs were observed to elicit a stronger cytokine response when compared to their bispecific counterpart.

### 7.6. Example 6: Production and Characterization of an Anti-Her2-Anti-CD3-Anti-CD2 TBM

7.6.1. Materials and Methods

A trispecific binding molecules targeting Her2, CD3 and CD2 was generated and analyzed for its potential to induce T cell-mediated apoptosis in tumor target cells. The TBM was compared to a bispecific construct targeting CD3 and Her2. Amino acid sequences of the bispecific and trispecific constructs used in this Example are shown in Table 24. The bispecific and trispecfic constructs used in this Example are schematically shown in FIG. 26.

TABLE 24
Amino Acid Sequences of Constucts of Example 6

| | | |
|---|---|---|
| | | SEQ |
| Component | Sequence | ID NO: |

Bispecific (FIG. 26A)
αHer2xαCD3

| | | |
|---|---|---|
| αHer2 Fab | | |
| HCDR1 (Combined) | GFNIKDTYIH | 774 |
| HCDR2 (Combined) | RIYPTNGYTRYADSVKG | 775 |
| HCDR3 (Combined) | WGGDGFYAMDY | 776 |
| HCDR1 (Kabat) | DTYIH | 777 |
| HCDR2 (Kabat) | RIYPTNGYTRYADSVKG | 775 |
| HCDR3 (Kabat) | WGGDGFYAMDY | 776 |
| HCDR1 (Chothia) | GFNIKDT | 778 |
| HCDR2 (Chothia) | YPTNGY | 779 |
| HCDR3 (Chothia) | WGGDGFYAMDY | 776 |
| HCDR1 (IMGT) | GFNIKDTY | 780 |
| HCDR2 (IMGT) | IYPTNGYT | 781 |
| HCDR3 (IMGT) | SRWGGDGFYAMDY | 782 |
| VH | EVQLVESGGGLVQPGGSLRLSCAASGFNIKDTYIHWVRQ | 783 |
| | APGKGLEWVARIYPTNGYTRYADSVKGRFTISADTSKNTA | |
| | YLQMNSLRAEDTAVYYCSRWGGDGFYAMDYWGQGTLV | |
| | TVSS | |
| LCDR1 (Combined) | RASQDVNTAVA | 784 |
| LCDR2 (Combined) | SASFLYS | 785 |
| LCDR3 (Combined) | QQHYTTPPT | 786 |
| LCDR1 (Kabat) | RASQDVNTAVA | 784 |
| LCDR2 (Kabat) | SASFLYS | 785 |
| LCDR3 (Kabat) | QQHYTTPPT | 786 |
| LCDR1 (Chothia) | SQDVNTA | 504 |
| LCDR2 (Chothia) | SAS | 518 |
| LCDR3 (Chothia) | HYTTPP | 787 |
| LCDR1 (IMGT) | QDVNTA | 788 |
| LCDR2 (IMGT) | SAS | 518 |
| LCDR3 (IMGT) | QQHYTTPPT | 786 |
| VL | DIQMTQSPSSLSASVGDRVTITCRASQDVNTAVAWYQQK | 789 |
| | PGKAPKLLIYSASFLYSGVPSRFSGSRSGTDFTLTISSLQP | |
| | EDFATYYCQQHYTTPPTFGQGTKVEIK | |
| αHer2 Light Chain | DIQMTGSPSSLSASVGDRVITICRASQDVNTAVAWYQQK | 790 |
| | PGKAPKWYSASFLYSGVPSRFSGSRSGTDFTLTISSLQP | |
| | EDFATYYCQQHYTTPPTFGQGTKVEIKRTVAAPSVFIFPP | |
| | SDEQLKSGTASVVCLLNNFYPREAKVQWKVDNALQSGN | |
| | SQESVTEQDSKDSTYSLSSTLTLSKADYEKHKVYACEVTH | |
| | QGLSSPVTKSFNRGEC | |
| αHer2 Heavy Chain | EVQLVESGGGLVQPGGSLRLSCAASGFNIKDTYIHWVRQ | 791 |
| | APGKGLEWVARIYPTNGYTRYADSVKGRFTISADTSKNTA | |
| | YLQMNSLRAEDTAVYYCSRWGGDGFYAMDYWGQGTLV | |
| | TVSSASTKGPSVFPLAPSSKSTSGGTAALGCLVKDYFPEP | |
| | VTVSWNSGALTSGVHTFPAVLQSSGLYSLSSVVTVPSSSL | |
| | GTQTYICNVNHKPSNTKVDKRVEPKSCDKTHTCPPCPAP | |
| | ELLGGPSVFLFPPKPKDTLMISRTPEVTCVVVAVSHEDPE | |
| | VKFNWYVDGVEVHNAKTKPREEQYASTYRVVSVLTVLHQ | |
| | DWLNGKEYKCKVSNKALAAPIEKTISKAKGQPREPQVCTL | |
| | PPSREEMTKNQVSLSCAVKGFYPSDIAVEWESNGQPENN | |
| | YKTTPPVLDSDGSFFLVSKLTVDKSRWQQGNVFSCSVMH | |
| | EALHNHYTQKSLSLSPGK | |
| αCD3 scFv-Fc | EVQLVESGGGLVQPGGSLKLSCAASGFTFNTYAMNWVR | 792 |
| | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSSGGGGSGGGGSGGGGSGGGGSQAVVTQ | |
| | EPSLTVSPGGTVTLTCRSSTGAVTTSNYANWVQQKPGQA | |
| | PRGLIGGTNKRAPWTPARFSGSLLGDKAALTLSGAQPED | |
| | EAEYFCALWYSNLWVFGGGTKLTVLGGGGSDKTHTCPP | |
| | CPAPELLGGPSVFLFPPKPKDTLMISRTPEVTCVVVAVSH | |
| | EDPEVKFNWYVDGVEVHNAKTKPREEQYASTYRVVSVLT | |
| | VLHQDWLNGKEYKCKVSNKALAAPIEKTISKAKGQPREPQ | |
| | VYTLPPCREEMTKNQVSLWCLVKGFYPSDIAVEWESNGQ | |
| | PENNYKTTPPVLDSDGSFFLYSKLTVDKSRWQQGNVFSC | |
| | SVMHEALHNRYTQKSLSLSPGK | |

Trispecific (FIG. 26B)
αHer2xαCD3xCD58IgV

| | | |
|---|---|---|
| αHer2 Light Chain | DIQMTQSPSSLSASVGDRVTITCRASQDVNTAVAWYQQK | 790 |
| | PGKAPKLLIYSASFLYSGVPSRFSGSRSGTDFTLTISSLQP | |
| | EDFATYYCQQHYTTPPTFGQGTKVEIKRTVAAPSVFIFPP | |
| | SDEQLKSGTASVVCLLNNFYPREAKVQWKVDNALQSGN | |
| | SQESVTEQDSKDSTYSLSSTLTLSKADYEKHKVYACEVTH | |
| | QGLSSPVTKSFNRGEC | |
| αHer2_Fab- | EVQLVESGGGLVQPGGSLRLSCAASGFNIKDTYIHWVRQ | 793 |
| αCD3_scFv-Fc | APGKGLEWVARIYPTNGYTRYADSVKGRFTISADTSKNTA | |
| | YLQMNSLRAEDTAVYYCSRWGGDGFYAMDYWGQGTLV | |
| | TVSSASTKGPSVFPLAPSSKSTSGGTAALGCLVKDYFPEP | |
| | VTVSWNSGALTSGVHTFPAVLQSSGLYSLSSVVTVPSSSL | |
| | GTQTYICNVNHKPSNTKVDKRVEPKSCGGGGSGGGGSE | |
| | VQLVESGGGLVQPGGSLKLSCAASGFTFNTYAMNWVRQ | |
| | ASGKGLEWVGRIRSKYNNYATYYADSVKDRFTISRDDSK | |
| | STLYLQMNSLKTEDTAVYYCVRHGNFGNSYVSWFAYWG | |
| | QGTLVTVSSGGGGSGGGGSGGGGSGGGGSQAVVTQEP | |
| | SLTVSPGGTVTLTCRSSTGAVTTSNYANWVQQKPGQAP | |
| | RGLIGGTNKRAPWTPARFSGSLLGDKAALTLSGAQPEDE | |
| | AEYFCALWYSNLWVFGGGTKLTVLGGGGSDKTHTCPPC | |
| | PAPELLGGPSVFLFPPKPKDTLMISRTPEVTCVVVAVSHE | |
| | DPEVKFNWYVDGVEVHNAKTKPREEQYASTYRVVSVLTV | |
| | LHQDWLNGKEYKCKVSNKALAAPIEKTISKAKGQPREPQV | |
| | CTLPPSREEMTKNQVSLSCAVKGFYPSDIAVEWESNGQP | |
| | ENNYKTTPPVLDSDGSFFLVSKLTVDKSRWQQGNVFSCS | |
| | VMHEALHNHYTQKSLSLSPGK | |
| CD58 IgV-Fc | SQQIYGVVYGNVTFHVPSNVPLKEVLWKKQKDKVAELEN | 744 |
| | SEFRAFSSFKNRVYIDTVSGSLTIYNLTSSDEDEYEMESP | |
| | NITDTMKFFLYVLESGGGGSDKTHTCPPCPAPELLGGPSV | |
| | FLFPPKPKDTLMISRTPEVTCVVVAVSHEDPEVKFNWYVD | |
| | GVEVHNAKTKPREEQYASTYRVVSVLTVLHQDWLNGKEY | |
| | KCKVSNKALAAPIEKTISKAKGQPREPQVYTLPPCREEMT | |
| | KNQVSLWCLVKGFYPSDIAVEWESNGQPENNYKTTPPVL | |
| | DSDGSFFLYSKLTVDKSRWQQGNVFSCSVMHEALHNRY | |
| | TQKSLSLSPGK | |

Re-directed T cell cytotoxicity assays were performed using the constructs as described in Example 1, using human huHer2-expressing HCC1954 target cells (cultured in RPMI medium with 10% FBS) and human pan T effector cells at an E:T ratio of 5:1 and an incubation of 48 hours.

Cytokine release assays were also performed. Secreted cytokines were measured in the supernatant of trispecific or bispecific treated effector and target cell co-cultures using huHer2-expressing HCC1954 target cells (cultured in RPMI medium with 10% FBS) and human pan T effector cells at an E:T ratio of 5:1 and an incubation of 24 hr.

7.6.2. Results

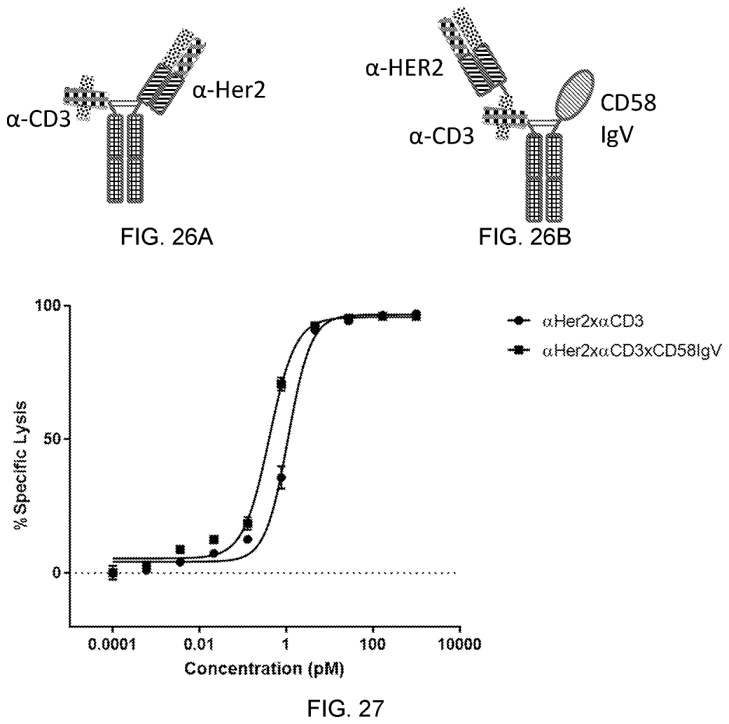

The results of the re-directed T cell cytotoxicity assay are shown in FIG. 27. The TBM showed equivalent to superior cytotoxic activity compared to the bispecific construct.

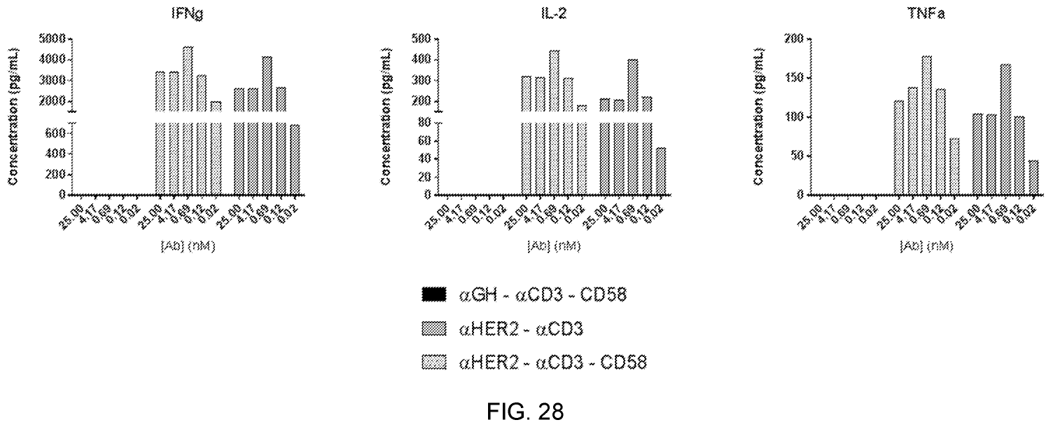

Results of the cytokine release assay are shown in FIG. 28. The trispecific construct targeting human CD3, human CD2 and human Her2 produced cytokine levels that were at least comparable to the bispecific molecule targeting human CD3 and human Her2.

### 7.7. Example 7: Production and Characterization of an Anti-Mesothelin-Anti-CD3-Anti-CD2 TBM

7.7.1. Materials and Methods

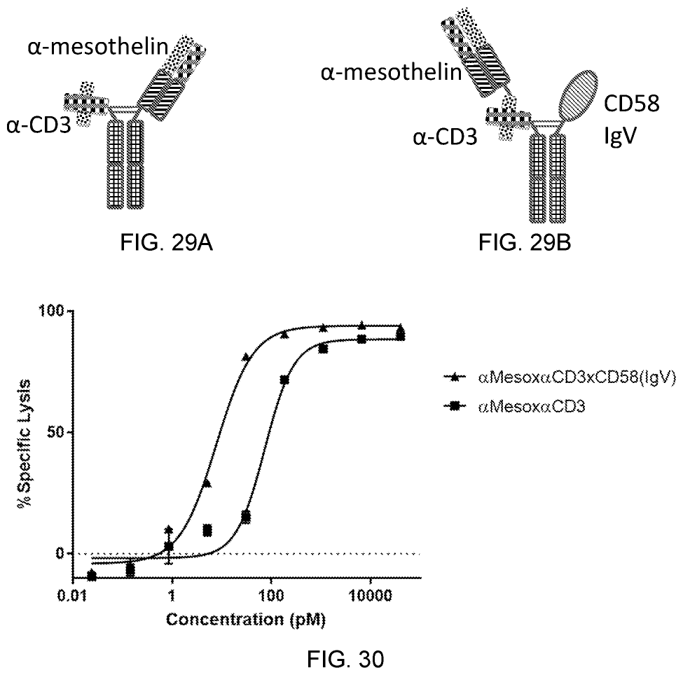

A trispecific binding molecules targeting mesothelin, CD3 and CD2 was generated and analyzed for its potential to induce T cell-mediated apoptosis in tumor target cells. The TBM was compared to a bispecific construct targeting CD3 and mesothelin. Amino acid sequences of the bispecific and trispecific constructs used in this Example are shown in Table 25. The bispecific and trispecfic constructs used in this Example are schematically shown in FIG. 29.

TABLE 25
Amino Acid Sequences of Constucts of Example 7

| | | |
|---|---|---|
| | | SEQ |
| Component | Sequence | ID NO: |

Bispecific (FIG. 29A)
αMesoxαCD3

| | | |
|---|---|---|
| αMesothelin Fab | | |
| HCDR1 (Combined) | GYTFTDYYMH | 794 |
| HCDR2 (Combined) | WINPNSGGTNYAQKFQG | 795 |
| HCDR3 (Combined) | GWDFDY | 796 |
| HCDR1 (Kabat) | DYYMH | 797 |
| HCDR2 (Kabat) | WINPNSGGTNYAQKFQG | 795 |
| HCDR3 (Kabat) | GWDFDY | 796 |
| HCDR1 (Chothia) | GYTFTDY | 644 |
| HCDR2 (Chothia) | NPNSGG | 798 |
| HCDR3 (Chothia) | GWDFDY | 796 |
| HCDR1 (IMGT) | GYTFTDYY | 799 |
| HCDR2 (IMGT) | INPNSGGT | 800 |
| HCDR3 (IMGT) | ASGWDFDY | 801 |
| VH | QVQLVQSGAEVEKPGASVKVSCKASGYTFTDYYMHWVR | 802 |
| | QAPGQGLEWMGWINPNSGGTNYAQKFQGRVTMTRDTSI | |
| | STAYMELSRLRSDDTAVYYCASGWDFDYWGQGTLVTVS | |
| | S | |
| LCDR1 (Combined) | RASQSIRYYLS | 803 |
| LCDR2 (Combined) | TASILQN | 804 |
| LCDR3 (Combined) | LQTYTTPD | 805 |
| LCDR1 (Kabat) | RASQSIRYYLS | 803 |
| LCDR2 (Kabat) | TASILQN | 804 |
| LCDR3 (Kabat) | LQTYTTPD | 805 |
| LCDR1 (Chothia) | SQSIRYY | 806 |
| LCDR2 (Chothia) | TAS | 807 |
| LCDR3 (Chothia) | TYTTP | 808 |
| LCDR1 (IMGT) | QSIRYY | 809 |
| LCDR2 (IMGT) | TASILQNGVP | 810 |
| LCDR3 (IMGT) | LQTYTTPD | 805 |
| VL | DIVMTQSPSSLSASVGDRVTITCRASQSIRYYLSWYQQKP | 811 |
| | GKAPKLLIYTASILQNGVPSRFSGSGSGTDFTLTISSLQPE | |
| | DFATYYCLQTYTTPDFGPGTKVEIK | |
| αMesothelin Light | DIVMTQSPSSLSASVGDRVTITCRASQSIRYYLSWYQQKP | 812 |
| Chain | GKAPKLLIYTASILQNGVPSRFSGSGSGTDFTLTISSLQPE | |
| | DFATYYCLQTYTTPDFGPGTKVEIKRTVAAPSVFIFPPSDE | |
| | QLKSGTASVVCLLNNFYPREAKVQWKVDNALQSGNSQE | |
| | SVTEQDSKDSTYSLSSTLTLSKADYEKHKVYACEVTHQGL | |
| | SSPVTKSFNRGEC | |
| αMesothelin Heavy | QVQLVQSGAEVEKPGASVKVSCKASGYTFTDYYMHWVR | 813 |
| Chain | QAPGQGLEWMGWINPNSGGTNYAQKFQGRVTMTRDTSI | |
| | STAYMELSRLRSDDTAVYYCASGWDFDYWGQGTLVTVS | |
| | SASTKGPSVFPLAPSSKSTSGGTAALGCLVKDYFPEPVTV | |
| | SWNSGALTSGVHTFPAVLQSSGLYSLSSVVTVPSSSLGT | |
| | QTYICNVNHKPSNTKVDKRVEPKSCDKTHTCPPCPAPELL | |
| | GGPSVFLFPPKPKDTLMISRTPEVTCVVVAVSHEDPEVKF | |
| | NWYVDGVEVHNAKTKPREEQYASTYRVVSVLTVLHQDW | |
| | LNGKEYKCKVSNKALAAPIEKTISKAKGQPREPQVCTLPP | |
| | SREEMTKNQVSLSCAVKGFYPSDIAVEWESNGQPENNYK | |
| | TTPPVLDSDGSFFLVSKLTVDKSRWQQGNVFSCSVMHEA | |
| | LHNHYTQKSLSLSPGK | |
| αCD3 scFv-Fc | EVQLVESGGGLVQPGGSLKLSCAASGFTFNTYAMNWVR | 792 |
| | QASGKGLEWVGRIRSKYNNYATYYADSVKDRFTISRDDS | |
| | KSTLYLQMNSLKTEDTAVYYCVRHGNFGNSYVSWFAYW | |
| | GQGTLVTVSSGGGGSGGGGSGGGGSGGGGSQAVVTQ | |
| | EPSLTVSPGGTVTLTCRSSTGAVTTSNYANWVQQKPGQA | |
| | PRGLIGGTNKRAPWTPARFSGSLLGDKAALTLSGAQPED | |
| | EAEYFCALWYSNLWVFGGGTKLTVLGGGGSDKTHTCPP | |
| | CPAPELLGGPSVFLFPPKPKDTLMISRTPEVTCVVVAVSH | |
| | EDPEVKFNWYVDGVEVHNAKTKPREEQYASTYRVVSVLT | |
| | VLHQDWLNGKEYKCKVSNKALAAPIEKTISKAKGQPREPQ | |
| | VYTLPPCREEMTKNQVSLWCLVKGFYPSDIAVEWESNGQ | |
| | PENNYKTTPPVLDSDGSFFLYSKLTVDKSRWQQGNVFSC | |
| | SVMHEALHNRYTQKSLSLSPGK | |

Trispecific (FIG. 29B)
αMesoxαCD3xCD58IgV

| | | |
|---|---|---|
| αMesothelin Light | DIVMTQSPSSLSASVGDRVTITCRASQSIRYYLSWYQQKP | 812 |
| Chain | GKAPKLLIYTASILQNGVPSRFSGSGSGTDFTLTISSLQPE | |
| | DFATYYCLQTYTTPDFGPGTKVEIKRTVAAPSVFIFPPSDE | |
| | QLKSGTASVVCLLNNFYPREAKVQWKVDNALQSGNSQE | |
| | SVTEQDSKDSTYSLSSTLTLSKADYEKHKVYACEVTHQGL | |
| | SSPVTKSFNRGEC | |
| αMesothelin_Fab- | QVQLVQSGAEVEKPGASVKVSCKASGYTFTDYYMHWVR | 814 |
| αCD3_scFv-Fc | QAPGQGLEWMGWINPNSGGTNYAQKFQGRVTMTRDTSI | |
| | STAYMELSRLRSDDTAVYYCASGWDFDYWGQGTLVTVS | |
| | SASTKGPSVFPLAPSSKSTSGGTAALGCLVKDYFPEPVTV | |
| | SWNSGALTSGVHTFPAVLQSSGLYSLSSVVTVPSSSLGT | |
| | QTYICNVNHKPSNTKVDKRVEPKSCGGGGSGGGGSEVQ | |
| | LVESGGGLVQPGGSLKLSCAASGFTFNTYAMNWVRQAS | |
| | GKGLEWVGRIRSKYNNYATYYADSVKDRFTISRDDSKSTL | |
| | YLQMNSLKTEDTAVYYCVRHGNFGNSYVSWFAYWGQGT | |
| | LVTVSSGGGGSGGGGSGGGGSGGGGSQAVVTQEPSLT | |
| | VSPGGTVTLTCRSSTGAVTTSNYANWVQQKPGQAPRGLI | |
| | GGTNKRAPWTPARFSGSLLGDKAALTLSGAQPEDEAEYF | |
| | CALWYSNLWVFGGGTKLTVLGGGGSDKTHTCPPCPAPE | |
| | LLGGPSVFLFPPKPKDTLMISRTPEVTCVVVAVSHEDPEV | |
| | KFNWYVDGVEVHNAKTKPREEQYGSTYRVVSVLTVLHQ | |
| | DWLNGKEYKCKVSNKALAAPIEKTISKAKGQPREPQVCTL | |
| | PPSRDELTKNQVSLSCAVKGFYPSDIAVEWESNGQPENN | |
| | YKTTPPVLDSDGSFFLVSKLTVDKSRWQQGNVFSCSVMH | |
| | EALHNHYTQKSLSLSPGK | |
| CD58 IgV-Fc | SQQIYGVVYGNVTFHVPSNVPLKEVLWKKQKDKVAELEN | 744 |
| | SEFRAFSSFKNRVYLDTVSGSLTIYNLTSSDEDEYEMESP | |
| | NITDTMKFFLYVLESGGGGSDKTHTCPPCPAPELLGGPSV | |
| | FLFPPKPKDTLMISRTPEVTCVVVAVSHEDPEVKFNWYVD | |
| | GVEVHNAKTKPREEQYASTYRVVSVLTVLHQDWLNGKEY | |
| | KCKVSNKALAAPIEKTISKAKGQPREPQVYTLPPCREEMT | |
| | KNQVSLWCLVKGFYPSDIAVEWESNGQPENNYKTTPPVL | |
| | DSDGSFFLYSKLTVDKSRWQQGNVFSCSVMHEALHNRY | |
| | TQKSLSLSPGK | |

Re-directed T cell cytotoxicity assays were performed using the constructs as described in Example 1, using human huMesothlin-expressing OVCAR8 target cells (cultured in DMEM medium with 10% FBS) and human pan T effector cells at an E:T ratio of 5:1 and an incubation of 48 hours.

Cytokine release assays were also performed. Secreted cytokines were measured in the supernatant of trispecific or bispecific treated effector and target cell co-cultures using huMesothelin-expressing Ovcar8 target cells (cultured in DMEM medium with 10% FBS) and human pan T effector cells at an E:T ratio of 5:1 and an incubation of 24 hr.

7.7.2. Results

The results of the re-directed T cell cytotoxicity assay are shown in FIG. 30. The TBM showed superior cytotoxic activity compared to the bispecific construct.

Results of the cytokine release assay are shown in FIG. 31. The trispecific construct targeting human CD3, human CD2 and human mesothelin produced cytokine levels that are at least comparable to the bispecific molecule targeting human CD3 and human mesothelin.

### 7.8. Example 8: Production and Characterization of an Anti-CD19-Anti-CD3-Anti-CD2 TBM with CD48 as the CD2 Binding Arm

7.8.1. Materials and Methods

7.8.1.1. Gene Construction, Expression and Purification of Bi- and Tri-Specific Binding Molecules

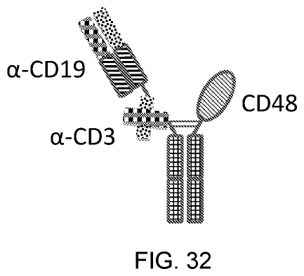

A trispecific construct is made as in Example 1, but with a CD48 moiety substituted for the CD58 moiety. The trispecific construct is represented schematically in FIG. 32. Amino acid sequences for the construct are shown in Table 26.

TABLE 26
Amino Acid Sequences of the Tri-specific Construct of Example 8
(CD19Fab/hCD3 scFv-CD48 ligand 1x1 format)

| | | SEQ |
|---|---|---|
| Component | Sequence | ID NO: |
| CD48 ligand (positions | QGHLVHMTVVSGSNVTLNISESLPENYKQLTWFYTFDQKI | 815 |
| 27-220 of UniProt | VEWDSRKSKYFESKFKGRVRLDPQSGALYISKVQKEDNS | |
| identifier P09326) | TYIMRVLKKTGNEQEWKIKLQVLDPVPKPVIKIEKIEDMDD | |
| | NCYLKLSCVIPGESVNYTWYGDKRPFPKELQNSVLETTLM | |
| | PHNYSRCYTCQVSNSVSSKNGTVCLSPPCTLARS | |
| αCD19 Light chain | EIVMTQSPATLSLSPGERATLSCRASQDISKYLNWYQQKP | 736 |
| | GQAPRLLIYHTSRLHSGIPARFSGSGSGTDYTLTISSLQPE | |
| | DFAVYFCQQGNTLPYTFGQGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| Full CD19-CD3 arm | QVQLQESGPGLVKPSETLSLTCTVSGVSLPDYGVSWIRQ | 738 |
| | PPGKGLEWIGVIWGSETTYYQSSLKSRVTISKDNSKNQVS | |
| | LKLSSVTAADTAVYYCAKHYYYGGSYAMDYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLG | |
| | TQTYICNVNHKPSNTKVDKRVEPKSCGGGGSGGGGSEV | |
| | QLVESGGGLVQPGGSLKLSCAASGFTFNTYAMNWVRQA | |
| | SGKGLEWVGRIRSKYNNYATYYADSVKDRFTISRDDSKS | |
| | TLYLQMNSLKTEDTAVYYCVRHGNFGNSYVSWFAYWGQ | |
| | GTLVTVSSGGGGSGGGGSGGGGSGGGGSQAVVTQEPS | |
| | LTVSPGGTVTLTCRSSTGAVTTSNYANVWQQKPGQAPR | |
| | GLIGGTNKRAPWTPARFSGSLLGDKAALTLSGAQPEDEA | |
| | EYFCALWYSNLWVFGGGTKLTVLGGGGSDKTHTCPPCP | |
| | APELLGGPSVFLFPPKPKDTLMISRTPEVTCVVVAVSHED | |
| | PEVKFNWYVDGVEVHNAKTKPREEQYGSTYRVVSVLTVL | |
| | HQDWLNGKEYKCKVSNKALAAPIEKTISKAKGQPREPQV | |
| | CTLPPSRDELTKNQVSLSCAVKGFYPSDIAVEWESNGQP | |
| | ENNYKTTPPVLDSDGSFFLVSKLTVDKSRWQQGNVFSCS | |
| | VMHEALHNHYTQKSLSLSPGK | |
| Full CD48 ligand arm | QGHLVHMTVVSGSNVTLNISESLPENYKQLTWFYTFDQKI | 816 |
| | VEWDSRKSKYFESKFKGRVRLDPQSGALYISKVQKEDNS | |
| | TYIMRVLKKTGNEQEWKIKLQVLDPVPKPVIKIEKIEDMDD | |
| | NCYLKLSCVIPGESVNYTWYGDKRPFPKELQNSVLETTLM | |
| | PHNYSRCYTCQVSNSVSSKNGTVCLSPPCTLARSGGGS | |
| | DKTHTCPPCPAPELLGGPSVFLFPPKPKDTLMISRTPEVT | |
| | CVVVDVSHEDPEVKFNWYVDGVEVHNAKTKPREEQYAS | |
| | TYRVVSVLTVLHQDWLNGKEYKCKVSNKALPAPIEKTISK | |
| | AKGQPREPQVYTLPPCREEMTKNQVSLWCLVKGFYPSDI | |
| | AVEWESNGQPENNYKTTPPVLDSDGSFFLYSKLTVDKSR | |
| | WQQGNVFSCSVMHEALHNRYTQKSLSLSPGK | |

The TBM is expressed and purified according to the protocol described in Example 1.

A re-directed T cell cytotoxicity assay, proliferation assay, cytokine release assay, and granzyme B ELISpot assay are performed as in Example 1, with the anti-CD19-anti-CD3 bispecfiic construct described in Example 1 as comparator molecule.

7.8.2. Results

7.8.2.1. Results

The TBM construct induces superior cytotoxicity in huCD19-expressing Nalm6 target cells compared to the bispecific construct in an assay performed with a final E:T ratio of 5:1 and either 24 or 48 hour incubation.

The TBM construct induces superior cytotoxic activity in huCD19-expressing Daudi target cells compared to the bispecific construct in an assay performed with a final E:T ratio of 5:1 and 48 hour incubation.

The TBM construct does not induce apoptosis of the negative control K562 cell line, which does not express human CD19, in an assay performed with a final E:T ratio of 5:1 and 48 hour incubation.

The TBM exhibits comparable or superior potency and efficacy compared to the bispecific construct in huCD19-expressing Nalm6 target cells when using human cryopreserved PBMCs that are thawed and rested overnight prior to use and with a final E:T ratio of 10:1 and 24 hour incubation.

The TBM construct induces superior cytotoxic activity in huCD19-expressing Nalm6 target cells compared to the bispecific when using human cryopreserved PBMCs from a patient diagnosed with acute myeloid leukemia (Hemacare #PB009C-1-AML) that are thawed and rested overnight prior to use and with a final E:T ratio of 10:1 and 24 hour incubation.

In a rechallenge assay with huCD19 expressing Nalm6 target cells, the TBM continues to induce apoptosis up to 120 hours with little apparent loss in activity across each rechallenge while the bispecific construct loses significant potency and activity with each subsequent target cell addition.

The trispecific construct mediates higher levels of CD25 and double positive expression (CD25 and CD69) compared to the bispecific construct in both the CD4 and CD8 compartments.

The trispecific construct mediates higher levels of PD1, LAG3 and TIM3 compared to the bispecific construct in both the CD4 and CD8 compartments.

The trispecific construct mediates a greater amount of central memory T cells compared to the bispecific construct.

The TBM induces proliferation of CD8+ T cells.

The trispecific molecule induces production of IFNγ, TNFα, IL2, IL6 and IL10 in human pan T effector cells in the presence of huCD19-expressing Nalm6 target cells.

The number of Granzyme B spotforming T cells is higher upon treatment with the CD2-CD3-CD19-targeting TBM than with the bispecific construct.

### 8. SPECIFIC EMBODIMENTS, CITATION OF REFERENCES

While various specific embodiments have been illustrated and described, it will be appreciated that various changes can be made without departing from the spirit and scope of the disclosure(s). The present disclosure is exemplified by the numbered embodiments set forth below.

1. A trispecific binding molecule (TBM), comprising:
(a) an antigen-binding module 1 (ABM1) that binds specifically to human CD2;
(b) an antigen-binding module 2 (ABM2) that binds specifically to a component of a human T-cell receptor (TCR) complex; and
(c) an antigen-binding module 3 (ABM3) that binds specifically to a human tumor-associated antigen (TAA),
   optionally wherein each antigen-binding module is capable of binding its respective target at the same time as each of the other antigen-binding modules is bound to its respective target.

2. The TBM of embodiment 1, wherein ABM1 comprises a receptor binding domain of a CD2 ligand.

3. The TBM of embodiment 2, wherein the CD2 ligand is CD58.

4. The TBM of embodiment 2, wherein the CD2 ligand is CD48.

5. The TBM of embodiment 1, wherein ABM1 is a CD58 moiety.

6. The TBM of embodiment 5, wherein the CD58 moiety comprises the amino acid sequence of CD58-5.

7. The TBM of embodiment 6, wherein the amino acid designated as J is a valine.

8. The TBM of embodiment 6, wherein the amino acid designated as J is a lysine.

9. The TBM of any one of embodiments 6 to 8, wherein the amino acid designated as O is a valine.

10. The TBM of any one of embodiments 6 to 8, wherein the amino acid designated as O is a glutamine.

11. The TBM of embodiment 5, wherein the CD58 moiety comprises the amino acid sequence of CD58-3.

12. The TBM of embodiment 11, wherein the amino acid designated as B is a phenylalanine.

13. The TBM of embodiment 11, wherein the amino acid designated as B is a serine.

14. The TBM of any one of embodiments 11 to 13, wherein the amino acid designated as J is a valine.

15. The TBM of any one of embodiments 11 to 13, wherein the amino acid designated as J is a lysine.

16. The TBM of any one of embodiments 11 to 15, wherein the amino acid designated as O is a valine.

17. The TBM of any one of embodiments 11 to 15, wherein the amino acid designated as O is a glutamine.

18. The TBM of any one of embodiments 11 to 17, wherein the amino acid designated as U is a valine.

19. The TBM of any one of embodiments 11 to 17, wherein the amino acid designated as U is a lysine.

20. The TBM of any one of embodiments 11 to 19, wherein the amino acid designated as X is a threonine.

21. The TBM of any one of embodiments 11 to 19, wherein the amino acid designated as X is a serine.

22. The TBM of any one of embodiments 11 to 21, wherein the amino acid designated as Z is a leucine.

23. The TBM of any one of embodiments 11 to 21, wherein the amino acid designated as X is a glycine.

24. The TBM of embodiment 2, wherein the CD58 moiety comprises amino acids 1-94 of CD58-2.

25. The TBM of embodiment 1, wherein ABM1 is a CD48 moiety.

26. The TBM of embodiment 25, wherein the CD48 moiety has at least 70% sequence identity to amino acids 27-220 of the amino acid sequence of Uniprot identifier P09326.

27. The TBM of embodiment 25, wherein the CD48 moiety has at least 80% sequence identity to amino acids 27-220 of the amino acid sequence of Uniprot identifier P09326.

28. The TBM of embodiment 25, wherein the CD48 moiety has at least 90% sequence identity to amino acids 27-220 of the amino acid sequence of Uniprot identifier P09326.

29. The TBM of embodiment 25, wherein the CD48 moiety has at least 95% sequence identity to f amino acids 27-220 of the amino acid sequence of Uniprot identifier P09326.

30. The TBM of embodiment 25, wherein the CD48 moiety has at least 99% sequence identity to amino acids 27-220 of the amino acid sequence of Uniprot identifier P09326.

31. The TBM of embodiment 1, wherein ABM1 is an anti-CD2 antibody or an antigen-binding domain thereof.

32. The TBM of embodiment 31, wherein ABM1 comprises the CDR sequences of CD2-1.

33. The TBM of embodiment 32, wherein ABM1 comprises the heavy and light chain variable sequences of CD2-1.

34. The TBM of embodiment 32, wherein ABM1 comprises the heavy and light chain variable sequences of hu1CD2-1.

35. The TBM of embodiment 32, wherein ABM1 comprises the heavy and light chain variable sequences of hu2CD2-1.

36. The TBM of embodiment 31, wherein ABM1 comprises the CDR sequences of Medi 507.

37. The TBM of embodiment 36, wherein ABM1 comprises the heavy and light chain variable sequences of Medi 507.

38. The TBM of any one of embodiments 1 to 37, wherein the component of the TCR complex is CD3.

39. The TBM of embodiment 38, wherein ABM2 is an anti-CD3 antibody or an antigen-binding domain thereof.

40. The TBM of embodiment 39, wherein ABM2 comprises the CDR sequences of CD3-1.

41. The TBM of embodiment 39, wherein ABM2 comprises the CDR sequences of CD3-2.

42. The TBM of embodiment 39, wherein ABM2 comprises the CDR sequences of CD3-3.

43. The TBM of embodiment 39, wherein ABM2 comprises the CDR sequences of CD3-4.

44. The TBM of embodiment 39, wherein ABM2 comprises the CDR sequences of CD3-5.

45. The TBM of embodiment 39, wherein ABM2 comprises the CDR sequences of CD3-6.

46. The TBM of embodiment 39, wherein ABM2 comprises the CDR sequences of CD3-7.

47. The TBM of embodiment 39, wherein ABM2 comprises the CDR sequences of CD3-8.

48. The TBM of embodiment 39, wherein ABM2 comprises the CDR sequences of CD3-9.

49. The TBM of embodiment 39, wherein ABM2 comprises the CDR sequences of CD3-10.

50. The TBM of embodiment 39, wherein ABM2 comprises the CDR sequences of CD3-11.

51. The TBM of embodiment 39, wherein ABM2 comprises the CDR sequences of CD3-12.

52. The TBM of embodiment 39, wherein ABM2 comprises the CDR sequences of CD3-13.

53. The TBM of embodiment 39, wherein ABM2 comprises the CDR sequences of CD3-14.

54. The TBM of embodiment 39, wherein ABM2 comprises the CDR sequences of CD3-15.

55. The TBM of embodiment 39, wherein ABM2 comprises the CDR sequences of CD3-16.

56. The TBM of embodiment 39, wherein ABM2 comprises the CDR sequences of CD3-17.

57. The TBM of embodiment 39, wherein ABM2 comprises the CDR sequences of CD3-18.

58. The TBM of embodiment 39, wherein ABM2 comprises the CDR sequences of CD3-19.

59. The TBM of embodiment 39, wherein ABM2 comprises the CDR sequences of CD3-20.

60. The TBM of embodiment 39, wherein ABM2 comprises the CDR sequences of CD3-21.

61. The TBM of any one of embodiment 40 to embodiment 60, wherein the CDRs are defined by Kabat numbering, as set forth in Table 7B.

62. The TBM of any one of embodiment 40 to embodiment 60, wherein the CDRs are defined by Chothia numbering, as set forth in Table 7C.

63. TBM of any one of embodiment 40 to embodiment 60, wherein the CDRs are defined by a combination of Kabat and Chothia numbering, as set forth in Table 7D.

64. The TBM of embodiment 39, wherein ABM2 comprises the heavy and light chain variable sequences of CD3-1, as set forth in Table 7A.

65. The TBM of embodiment 39, wherein ABM2 comprises the heavy and light chain variable sequences of CD3-2, as set forth in Table 7A.

66. The TBM of embodiment 39, wherein ABM2 comprises the heavy and light chain variable sequences of CD3-3, as set forth in Table 7A.

67. The TBM of embodiment 39, wherein ABM2 comprises the heavy and light chain variable sequences of CD3-4, as set forth in Table 7A.

68. The TBM of embodiment 39, wherein ABM2 comprises the heavy and light chain variable sequences of CD3-5, as set forth in Table 7A.

69. The TBM of embodiment 39, wherein ABM2 comprises the heavy and light chain variable sequences of CD3-6, as set forth in Table 7A.

70. The TBM of embodiment 39, wherein ABM2 comprises the heavy and light chain variable sequences of CD3-7, as set forth in Table 7A.

71. The TBM of embodiment 39, wherein ABM2 comprises the heavy and light chain variable sequences of CD3-8, as set forth in Table 7A.

72. The TBM of embodiment 39, wherein ABM2 comprises the heavy and light chain variable sequences of CD3-9, as set forth in Table 7A.

73. The TBM of embodiment 39, wherein ABM2 comprises the heavy and light chain variable sequences of CD3-10, as set forth in Table 7A.

74. The TBM of embodiment 39, wherein ABM2 comprises the heavy and light chain variable sequences of CD3-11, as set forth in Table 7A.

75. The TBM of embodiment 39, wherein ABM2 comprises the heavy and light chain variable sequences of CD3-12, as set forth in Table 7A.

76. The TBM of embodiment 39, wherein ABM2 comprises the heavy and light chain variable sequences of CD3-13, as set forth in Table 7A.

77. The TBM of embodiment 39, wherein ABM2 comprises the heavy and light chain variable sequences of CD3-14, as set forth in Table 7A.

78. The TBM of embodiment 39, wherein ABM2 comprises the heavy and light chain variable sequences of CD3-15, as set forth in Table 7A.

79. The TBM of embodiment 39, wherein ABM2 comprises the heavy and light chain variable sequences of CD3-16, as set forth in Table 7A.

80. The TBM of embodiment 39, wherein ABM2 comprises the heavy and light chain variable sequences of CD3-17, as set forth in Table 7A.

81. The TBM of embodiment 39, wherein ABM2 comprises the heavy and light chain variable sequences of CD3-18, as set forth in Table 7A.

82. The TBM of embodiment 39, wherein ABM2 comprises the heavy and light chain variable sequences of CD3-19, as set forth in Table 7A.

83. The TBM of embodiment 39, wherein ABM2 comprises the heavy and light chain variable sequences of CD3-20, as set forth in Table 7A.

84. The TBM of embodiment 39, wherein ABM2 comprises the heavy and light chain variable sequences of CD3-21, as set forth in Table 7A.

85. The TBM of embodiment 39, wherein ABM2 comprises the CDR sequences of OKT3.

86. The TBM of embodiment 85, wherein the CDRs are defined by Kabat numbering, as set forth in Table 23B.

87. The TBM of embodiment 85, wherein the CDRs are defined by Chothia numbering, as set forth in Table 23B.

88. The TBM of embodiment 85, wherein the CDRs are defined by IMGT numbering, as set forth in Table 23B.

89. TBM of embodiment 85, wherein the CDRs are defined by a combination of Kabat and Chothia numbering, as set forth in Table 23B.

90. TBM of embodiment 85, wherein ABM2 comprises the heavy and light chain variable sequences of OKT3, as set forth in Table 23B.

91. The TBM of any one of embodiments 1 to 35, wherein the component of the TCR complex is TCR-α, TCR-β, or a TCR-α/β dimer.

92. The TBM of embodiment 91, wherein ABM2 is an antibody or an antigen-binding domain thereof.

93. The TBM of embodiment 92, wherein ABM2 comprises the CDR sequences of BMA031.

94. The TBM of embodiment 93, wherein the CDR sequences are defined as in Table 8.

95. The TBM of embodiment 93, wherein the CDR sequences are defined by Kabat numbering, as set forth in Table 23A.

96. The TBM of embodiment 93, wherein the CDR sequences are defined by IMGT numbering, as set forth in Table 23A.

97. The TBM of embodiment 93, wherein the CDR sequences are defined by Chothia numbering, as set forth in Table 23A.

98. The TBM of embodiment 93, wherein the CDR sequences are defined by a combination of Kabat and Chothia numbering, as set forth in Table 23A.

99. The TBM of embodiment 93, wherein ABM2 comprises the heavy and light chain variable sequences of BMA031.

100. The TBM of any any one of embodiments 1 to 35, wherein the component of the TCR complex is TCR-γ, TCR-δ, or a TCR-γ/δ dimer.

101. The TBM of embodiment 100, wherein ABM2 is an antibody or an antigen-binding domain thereof.

102. The TBM of embodiment 101 wherein ABM2 comprises the CDR sequences of OTCS1.

103. The TBM of embodiment 102, wherein the CDR sequences are defined by Kabat numbering.

104. The TBM of embodiment 102, wherein the CDR sequences are defined by Chothia numbering.

105. The TBM of embodiment 102, wherein the CDR sequences are defined by a combination of Kabat and Chothia numbering.

106. The TBM of embodiment 102, wherein ABM2 comprises the heavy and light chain variable sequences of OTCS1.

107. The TBM of any one of embodiments 1 to 106 wherein ABM3 is an anti-TAA antibody or an antigen-binding domain thereof.

108. The TBM of embodiment 107, wherein the TAA is TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, FLT3, TAAG72, CD22, CD33, GD2, BCMA, gp100Tn, FAP, tyrosinase, EPCAM, CEA, Igf-I receptor, EphB2, mesothelin, Cadherin17, CD32b, EGFRvIII, GPNMB, GPR64, HER3, LRP6, LYPD8, NKG2D, SLC34A2, SLC39A6, SLITRK6, or TACSTD2.

109. The TBM of embodiment 108, wherein the anti-TAA antibody or antigen-binding domain thereof has the CDR sequences of an antibody set forth in Table 11.

110. The TBM of embodiment 108, wherein the anti-TAA antibody or antigen-binding domain thereof has the heavy and light chain variable region sequences of an antibody set forth in Table 11.

111. The TBM of embodiment 109 or embodiment 110, wherein the anti-TAA antibody or antigen-binding domain thereof binds to CD22.

112. The TBM of embodiment 109 or embodiment 110, wherein the anti-TAA antibody or antigen-binding domain thereof binds to CS1.

113. The TBM of embodiment 109 or embodiment 110, wherein the anti-TAA antibody or antigen-binding domain thereof binds to CD33.

114. The TBM of embodiment 109 or embodiment 110, wherein the anti-TAA antibody or antigen-binding domain thereof binds to GD2.

115. The TBM of embodiment 109 or embodiment 110, wherein the anti-TAA antibody or antigen-binding domain thereof binds to BCMA.

116. The TBM of embodiment 109 or embodiment 110, wherein the anti-TAA antibody or antigen-binding domain thereof binds to Tn.

117. The TBM of embodiment 109 or embodiment 110, wherein the anti-TAA antibody or antigen-binding domain thereof binds to PSMA.

118. The TBM of embodiment 109 or embodiment 110, wherein the anti-TAA antibody or antigen-binding domain thereof binds to ROR1.

119. The TBM of embodiment 109 or embodiment 110, wherein the anti-TAA antibody or antigen-binding domain thereof binds to FLT3.

120. The TBM of embodiment 109 or embodiment 110, wherein the anti-TAA antibody or antigen-binding domain thereof binds to TAAG72.

121. The TBM of embodiment 109 or embodiment 110, wherein the anti-TAA antibody or antigen-binding domain thereof binds to FAP.

122. The TBM of embodiment 109 or embodiment 110, wherein the anti-TAA antibody or antigen-binding domain thereof binds to CD38.

123. The TBM of embodiment 109 or embodiment 110, wherein the anti-TAA antibody or antigen-binding domain thereof binds to CD44v6.

124. The TBM of embodiment 109 or embodiment 110, wherein the anti-TAA antibody or antigen-binding domain thereof binds to CEA.

125. The TBM of embodiment 109 or embodiment 110, wherein the anti-TAA antibody or antigen-binding domain thereof binds to EPCAM.

126. The TBM of embodiment 109 or embodiment 110, wherein the anti-TAA antibody or antigen-binding domain thereof binds to PRSS21.

127. The TBM of embodiment 109 or embodiment 110, wherein the anti-TAA antibody or antigen-binding domain thereof binds to B7H3.

128. The TBM of embodiment 109 or embodiment 110, wherein the anti-TAA antibody or antigen-binding domain thereof binds to KIT.

129. The TBM of embodiment 109 or embodiment 110, wherein the anti-TAA antibody or antigen-binding domain thereof binds to IL-13Ra2.

130. The TBM of embodiment 109 or embodiment 110, wherein the anti-TAA antibody or antigen-binding domain thereof binds to CD30.

131. The TBM of embodiment 109 or embodiment 110, wherein the anti-TAA antibody or antigen-binding domain thereof binds to GD3.

132. The TBM of embodiment 109 or embodiment 110, wherein the anti-TAA antibody or antigen-binding domain thereof binds to CD171.

133. The TBM of embodiment 109 or embodiment 110, wherein the anti-TAA antibody or antigen-binding domain thereof binds to IL-11Ra.

134. The TBM of embodiment 109 or embodiment 110, wherein the anti-TAA antibody or antigen-binding domain thereof binds to PSCA.

135. The TBM of embodiment 109 or embodiment 110, wherein the anti-TAA antibody or antigen-binding domain thereof binds to VEGFR2.

136. The TBM of embodiment 109 or embodiment 110, wherein the anti-TAA antibody or antigen-binding domain thereof binds to LewisY.

137. The TBM of embodiment 109 or embodiment 110, wherein the anti-TAA antibody or antigen-binding domain thereof binds to CD24.

138. The TBM of embodiment 109 or embodiment 110, wherein the anti-TAA antibody or antigen-binding domain thereof binds to PDGFR-beta.

139. The TBM of embodiment 109 or embodiment 110, wherein the anti-TAA antibody or antigen-binding domain thereof binds to SSEA-4.

140. The TBM of embodiment 109 or embodiment 110, wherein the anti-TAA antibody or antigen-binding domain thereof binds to CD20.

141. The TBM of embodiment 109 or embodiment 110, wherein the anti-TAA antibody or antigen-binding domain thereof binds to folate receptor alpha.

142. The TBM of embodiment 109 or embodiment 110, wherein the anti-TAA antibody or antigen-binding domain thereof binds to ERBB2.

143. The TBM of embodiment 109 or embodiment 110, wherein the anti-TAA antibody or antigen-binding domain thereof binds to MUC1.

144. The TBM of embodiment 109 or embodiment 110, wherein the anti-TAA antibody or antigen-binding domain thereof binds to EGFR.

145. The TBM of embodiment 109 or embodiment 110, wherein the anti-TAA antibody or antigen-binding domain thereof binds to NCAM.

146. The TBM of embodiment 109 or embodiment 110, wherein the anti-TAA antibody or antigen-binding domain thereof binds to ephrin B2

147. The TBM of embodiment 109 or embodiment 110, wherein the anti-TAA antibody or antigen-binding domain thereof binds to IGF-I receptor.

148. The TBM of embodiment 109 or embodiment 110, wherein the anti-TAA antibody or antigen-binding domain thereof binds to CAIX.

149. The TBM of embodiment 109 or embodiment 110, wherein the anti-TAA antibody or antigen-binding domain thereof binds to LMP2.

150. The TBM of embodiment 109 or embodiment 110, wherein the anti-TAA antibody or antigen-binding domain thereof binds to gp100.

151. The TBM of embodiment 109 or embodiment 110, wherein the anti-TAA antibody or antigen-binding domain thereof binds to tyrosinase.

152. The TBM of embodiment 109 or embodiment 110, wherein the anti-TAA antibody or antigen-binding domain thereof binds to ephA2.

153. The TBM of embodiment 109 or embodiment 110, wherein the anti-TAA antibody or antigen-binding domain thereof binds to mesothelin.

154. The TBM of embodiment 109 or embodiment 110, wherein the anti-TAA antibody or antigen-binding domain thereof binds to ALK.

155. The TBM of embodiment 109 or embodiment 110, wherein the anti-TAA antibody or antigen-binding domain thereof binds to CD19.

156. The TBM of embodiment 109 or embodiment 110, wherein the anti-TAA antibody or antigen-binding domain thereof binds to CD97.

157. The TBM of embodiment 109 or embodiment 110, wherein the anti-TAA antibody or antigen-binding domain thereof binds to CLDN6.

158. The TBM of embodiment 109 or embodiment 110, wherein the anti-TAA antibody or antigen-binding domain thereof binds to EGFRvIII.

159. The TBM of embodiment 109 or embodiment 110, wherein the anti-TAA antibody or antigen-binding domain thereof binds to folate receptor beta.

160. The TBM of embodiment 109 or embodiment 110, wherein the anti-TAA antibody or antigen-binding domain thereof binds to GloboH.

161. The TBM of embodiment 109 or embodiment 110, wherein the anti-TAA antibody or antigen-binding domain thereof binds to GPRC5D.

162. The TBM of embodiment 109 or embodiment 110, wherein the anti-TAA antibody or antigen-binding domain thereof binds to HMWMAA.

163. The TBM of embodiment 109 or embodiment 110, wherein the anti-TAA antibody or antigen-binding domain thereof binds to LRP6.

164. The TBM of embodiment 109 or embodiment 110, wherein the anti-TAA antibody or antigen-binding domain thereof binds to NY-BR-1.

165. The TBM of embodiment 109 or embodiment 110, wherein the anti-TAA antibody or antigen-binding domain thereof binds to PLAC1.

166. The TBM of embodiment 109 or embodiment 110, wherein the anti-TAA antibody or antigen-binding domain thereof binds to polysialic acid.

167. The TBM of embodiment 109 or embodiment 110, wherein the anti-TAA antibody or antigen-binding domain thereof binds to TEM1/CD248.

168. The TBM of embodiment 109 or embodiment 110, wherein the anti-TAA antibody or antigen-binding domain thereof binds to TSHR.

169. The TBM of embodiment 107, wherein the TAA is CD19.

170. The TBM of embodiment 169, wherein the anti-TAA antibody or antigen-binding domain thereof comprises heavy chain CDRs having the amino acid sequences of CD19-H1, CD19-H2A, and CD19-H3 as set forth in Table 13 and light chain CDRs having the amino acid sequences of CD19-L1, CD19-L2, and CD19-L3 as set forth in Table 13.

171. The TBM of embodiment 170, wherein the anti-TAA antibody or antigen-binding domain thereof comprises a heavy chain variable region having the amino acid sequences of VHA as set forth in Table 13 and a light chain variable region having the amino acid sequences of VLA as set forth in Table 13.

172. The TBM of embodiment 169, wherein the anti-TAA antibody or antigen-binding domain thereof comprises heavy chain CDRs having the amino acid sequences of CD19-H1, CD19-H2B, and CD19-H3 as set forth in Table 13 and light chain CDRs having the amino acid sequences of CD19-L1, CD19-L2, and CD19-L3 as set forth in Table 13.

173. The TBM of embodiment 172, wherein the anti-TAA antibody or antigen-binding domain thereof comprises a heavy chain variable region having the amino acid sequences of VHB as set forth in Table 13 and a light chain variable region having the amino acid sequences of VLB as set forth in Table 13.

174. The TBM of embodiment 169, wherein the anti-TAA antibody or antigen-binding domain thereof comprises heavy chain CDRs having the amino acid sequences of CD19-H1, CD19-H2C, and CD19-H3 as set forth in Table 13 and light chain CDRs having the amino acid sequences of CD19-L1, CD19-L2, and CD19-L3 as set forth in Table 13.

175. The TBM of embodiment 174, wherein the anti-TAA antibody or antigen-binding domain thereof comprises a heavy chain variable region having the amino acid sequences of VHC as set forth in Table 13 and a light chain variable region having the amino acid sequences of VLB as set forth in Table 13.

176. The TBM of embodiment 169, wherein the anti-TAA antibody or antigen-binding domain thereof comprises heavy chain CDRs having the amino acid sequences of CD19-H1, CD19-H2D, and CD19-H3 as set forth in Table 13 and light chain CDRs having the amino acid sequences of CD19-L1, CD19-L2, and CD19-L3 as set forth in Table 13.

177. The TBM of embodiment 176, wherein the anti-TAA antibody or antigen-binding domain thereof comprises a heavy chain variable region having the amino acid sequences of VHD as set forth in Table 13 and a light chain variable region having the amino acid sequences of VLB as set forth in Table 13.

178. The TBM of embodiment 107, wherein the TAA is Her2.

179. The TBM of embodiment 178, wherein ABM3 comprises the CDR sequences of the VH and VL sequences of the anti-Her2 Fab set forth in Table 24.

180. The TBM of embodiment 179, wherein the CDRs are defined by Kabat numbering, as set forth in Table 24.

181. The TBM of embodiment 179, wherein the CDRs are defined by Chothia numbering, as set forth in Table 24.

182. The TBM of embodiment 179, wherein the CDRs are defined by IMGT numbering, as set forth in Table 24.

183. The TBM of embodiment 179, wherein the CDRs are defined by a combination of Kabat and Chothia numbering, as set forth in Table 24.

184. The TBM of embodiment 178, wherein ABM3 comprises the VH and VL sequences of the anti-Her2 Fab set forth in Table 24.

185. The TBM of embodiment 107, wherein the TAA is mesothelin.

186. The TBM of embodiment 185, wherein ABM3 comprises the CDR sequences of the VH and VL sequences of the anti-mesothelin Fab set forth in Table 25.

187. The TBM of embodiment 186, wherein the CDRs are defined by Kabat numbering, as set forth in Table 25.

188. The TBM of embodiment 186, wherein the CDRs are defined by Chothia numbering, as set forth in Table 25.

189. The TBM of embodiment 186, wherein the CDRs are defined by IMGT numbering, as set forth in Table 25.

190. The TBM of embodiment 186, wherein the CDRs are defined by a combination of Kabat and Chothia numbering, as set forth in Table 25.

191. The TBM of embodiment 185, wherein ABM3 comprises the VH and VL sequences of the anti-mesothelin Fab set forth in Table 25.

192. The TBM of embodiment 107, wherein the TAA is BCMA.

193. The TBM of embodiment 192, wherein ABM3 comprises the CDR sequences of BCMA-1.

194. The TBM of embodiment 192, wherein ABM3 comprises the CDR sequences of BCMA-2.

195. The TBM of embodiment 192, wherein ABM3 comprises the CDR sequences of BCMA-3.

196. The TBM of embodiment 192, wherein ABM3 comprises the CDR sequences of BCMA-4.

197. The TBM of embodiment 192, wherein ABM3 comprises the CDR sequences of BCMA-5.

198. The TBM of embodiment 192, wherein ABM3 comprises the CDR sequences of BCMA-6.

199. The TBM of embodiment 192, wherein ABM3 comprises the CDR sequences of BCMA-7.

200. The TBM of embodiment 192, wherein ABM3 comprises the CDR sequences of BCMA-8.

201. The TBM of embodiment 192, wherein ABM3 comprises the CDR sequences of BCMA-9.

202. The TBM of embodiment 178, wherein ABM3 comprises the CDR sequences of BCMA-10.

203. The TBM of embodiment 192, wherein ABM3 comprises the CDR sequences of BCMA-11.

204. The TBM of embodiment 192, wherein ABM3 comprises the CDR sequences of BCMA-12.

205. The TBM of embodiment 192, wherein ABM3 comprises the CDR sequences of BCMA-13.

206. The TBM of embodiment 192, wherein ABM3 comprises the CDR sequences of BCMA-14.

207. The TBM of embodiment 192, wherein ABM3 comprises the CDR sequences of BCMA-15.

208. The TBM of embodiment 192, wherein ABM3 comprises the CDR sequences of BCMA-16.

209. The TBM of embodiment 192, wherein ABM3 comprises the CDR sequences of BCMA-17.

210. The TBM of embodiment 192, wherein ABM3 comprises the CDR sequences of BCMA-18.

211. The TBM of embodiment 192, wherein ABM3 comprises the CDR sequences of BCMA-19.

212. The TBM of embodiment 192, wherein ABM3 comprises the CDR sequences of BCMA-20.

213. The TBM of embodiment 192, wherein ABM3 comprises the CDR sequences of BCMA-21.

214. The TBM of embodiment 192, wherein ABM3 comprises the CDR sequences of BCMA-22.

215. The TBM of embodiment 192, wherein ABM3 comprises the CDR sequences of BCMA-23.

216. The TBM of embodiment 192, wherein ABM3 comprises the CDR sequences of BCMA-24.

217. The TBM of embodiment 192, wherein ABM3 comprises the CDR sequences of BCMA-25.

218. The TBM of embodiment 192, wherein ABM3 comprises the CDR sequences of BCMA-26.

219. The TBM of embodiment 192, wherein ABM3 comprises the CDR sequences of BCMA-27.

220. The TBM of embodiment 192, wherein ABM3 comprises the CDR sequences of BCMA-28.

221. The TBM of embodiment 192, wherein ABM3 comprises the CDR sequences of BCMA-29.

222. The TBM of embodiment 192, wherein ABM3 comprises the CDR sequences of BCMA-30.

223. The TBM of embodiment 192, wherein ABM3 comprises the CDR sequences of BCMA-31.

224. The TBM of embodiment 192, wherein ABM3 comprises the CDR sequences of BCMA-32.

225. The TBM of embodiment 192, wherein ABM3 comprises the CDR sequences of BCMA-33.

226. The TBM of embodiment 192, wherein ABM3 comprises the CDR sequences of BCMA-34.

227. The TBM of embodiment 192, wherein ABM3 comprises the CDR sequences of BCMA-35.

228. The TBM of embodiment 192, wherein ABM3 comprises the CDR sequences of BCMA-36.

229. The TBM of embodiment 192, wherein ABM3 comprises the CDR sequences of BCMA-37.

230. The TBM of embodiment 192, wherein ABM3 comprises the CDR sequences of BCMA-38.

231. The TBM of embodiment 192, wherein ABM3 comprises the CDR sequences of BCMA-39.

232. The TBM of embodiment 192, wherein ABM3 comprises the CDR sequences of BCMA-40.

233. The TBM of any one of embodiments 193 to 232, wherein the CDRs are defined by Kabat numbering, as set forth in Table 12B and 12E.

234. The TBM of any one of embodiments 193 to 232, wherein the CDRs are defined by Chothia numbering, as set forth in Table 12C and 12F.

235. TBM of any one of embodiments 193 to 232, wherein the CDRs are defined by a combination of Kabat and Chothia numbering, as set forth in Table 12D and 12G.

236. The TBM of embodiment 192, wherein ABM3 comprises the heavy and light chain variable sequences of BCMA-1, as set forth in Table 12A.

237. The TBM of embodiment 192, wherein ABM3 comprises the heavy and light chain variable sequences of BCMA-2, as set forth in Table 12A.

238. The TBM of embodiment 192, wherein ABM3 comprises the heavy and light chain variable sequences of BCMA-3, as set forth in Table 12A.

239. The TBM of embodiment 192, wherein ABM3 comprises the heavy and light chain variable sequences of BCMA-4, as set forth in Table 12A.

240. The TBM of embodiment 192, wherein ABM3 comprises the heavy and light chain variable sequences of BCMA-5, as set forth in Table 12A.

241. The TBM of embodiment 192, wherein ABM3 comprises the heavy and light chain variable sequences of BCMA-6, as set forth in Table 12A.

242. The TBM of embodiment 192, wherein ABM3 comprises the heavy and light chain variable sequences of BCMA-7, as set forth in Table 12A.

243. The TBM of embodiment 192, wherein ABM3 comprises the heavy and light chain variable sequences of BCMA-8, as set forth in Table 12A.

244. The TBM of embodiment 192, wherein ABM3 comprises the heavy and light chain variable sequences of BCMA-9, as set forth in Table 12A.

245. The TBM of embodiment 192, wherein ABM3 comprises the heavy and light chain variable sequences of BCMA-10, as set forth in Table 12A.

246. The TBM of embodiment 192, wherein ABM3 comprises the heavy and light chain variable sequences of BCMA-11, as set forth in Table 12A.

247. The TBM of embodiment 192, wherein ABM3 comprises the heavy and light chain variable sequences of BCMA-12, as set forth in Table 12A.

248. The TBM of embodiment 192, wherein ABM3 comprises the heavy and light chain variable sequences of BCMA-13, as set forth in Table 12A.

249. The TBM of embodiment 192, wherein ABM3 comprises the heavy and light chain variable sequences of BCMA-14, as set forth in Table 12A.

250. The TBM of embodiment 192, wherein ABM3 comprises the heavy and light chain variable sequences of BCMA-15, as set forth in Table 12A.

251. The TBM of embodiment 192, wherein ABM3 comprises the heavy and light chain variable sequences of BCMA-16, as set forth in Table 12A.

252. The TBM of embodiment 192, wherein ABM3 comprises the heavy and light chain variable sequences of BCMA-17, as set forth in Table 12A.

253. The TBM of embodiment 192, wherein ABM3 comprises the heavy and light chain variable sequences of BCMA-18, as set forth in Table 12A.

254. The TBM of embodiment 192, wherein ABM3 comprises the heavy and light chain variable sequences of BCMA-19, as set forth in Table 12A.

255. The TBM of embodiment 192, wherein ABM3 comprises the heavy and light chain variable sequences of BCMA-20, as set forth in Table 12A.

256. The TBM of embodiment 192, wherein ABM3 comprises the heavy and light chain variable sequences of BCMA-21, as set forth in Table 12A.

257. The TBM of embodiment 192, wherein ABM3 comprises the heavy and light chain variable sequences of BCMA-22, as set forth in Table 12A.

258. The TBM of embodiment 192, wherein ABM3 comprises the heavy and light chain variable sequences of BCMA-23, as set forth in Table 12A.

259. The TBM of embodiment 192, wherein ABM3 comprises the heavy and light chain variable sequences of BCMA-24, as set forth in Table 12A.

260. The TBM of embodiment 192, wherein ABM3 comprises the heavy and light chain variable sequences of BCMA-25, as set forth in Table 12A.

261. The TBM of embodiment 192, wherein ABM3 comprises the heavy and light chain variable sequences of BCMA-26, as set forth in Table 12A.

262. The TBM of embodiment 192, wherein ABM3 comprises the heavy and light chain variable sequences of BCMA-27, as set forth in Table 12A.

263. The TBM of embodiment 192, wherein ABM3 comprises the heavy and light chain variable sequences of BCMA-28, as set forth in Table 12A.

264. The TBM of embodiment 192, wherein ABM3 comprises the heavy and light chain variable sequences of BCMA-29, as set forth in Table 12A.

265. The TBM of embodiment 192, wherein ABM3 comprises the heavy and light chain variable sequences of BCMA-30, as set forth in Table 12A.

266. The TBM of embodiment 192, wherein ABM3 comprises the heavy and light chain variable sequences of BCMA-31, as set forth in Table 12A.

267. The TBM of embodiment 192, wherein ABM3 comprises the heavy and light chain variable sequences of BCMA-32, as set forth in Table 12A.

268. The TBM of embodiment 192, wherein ABM3 comprises the heavy and light chain variable sequences of BCMA-33, as set forth in Table 12A.

269. The TBM of embodiment 192, wherein ABM3 comprises the heavy and light chain variable sequences of BCMA-34, as set forth in Table 12A.

270. The TBM of embodiment 192, wherein ABM3 comprises the heavy and light chain variable sequences of BCMA-35, as set forth in Table 12A.

271. The TBM of embodiment 192, wherein ABM3 comprises the heavy and light chain variable sequences of BCMA-36, as set forth in Table 12A.

272. The TBM of embodiment 192, wherein ABM3 comprises the heavy and light chain variable sequences of BCMA-37, as set forth in Table 12A.

273. The TBM of embodiment 192, wherein ABM3 comprises the heavy and light chain variable sequences of BCMA-38, as set forth in Table 12A.

274. The TBM of embodiment 192, wherein ABM3 comprises the heavy and light chain variable sequences of BCMA-39, as set forth in Table 12A.

275. The TBM of embodiment 192, wherein ABM3 comprises the heavy and light chain variable sequences of BCMA-40, as set forth in Table 12A.

276. The TBM of embodiment 192, wherein ABM3 comprises the heavy and light chain variable sequences of BCMA-40, as set forth in Table 12A.

277. The TBM of any one of embodiments 1 to 106, wherein if TAA is a receptor, ABM3 comprises a receptor binding domain of a ligand of the receptor, and if TAA is a ligand, ABM3 comprises a ligand binding domain of a receptor of the ligand.

278. The TBM of embodiment 277, wherein the TAA is TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, FLT3, TAAG72, CD22, CD33, GD2, BCMA, gp100Tn, FAP, tyrosinase, EPCAM, CEA, Igf-I receptor, EphB2, mesothelin, Cadherin17, CD32b, EGFRvIII, GPNMB, GPR64, HER3, LRP6, LYPD8, NKG2D, SLC34A2, SLC39A6, SLITRK6, or TACSTD2.

279. The TBM of embodiment 278, wherein the TAA is CD22.

280. The TBM of embodiment 278, wherein the TAA is CS1.

281. The TBM of embodiment 278, wherein the TAA is CD33.

282. The TBM of embodiment 278, wherein the TAA is GD2.

283. The TBM of embodiment 278, wherein the TAA is BCMA.

284. The TBM of embodiment 278, wherein the TAA is Tn.

285. The TBM of embodiment 278, wherein the TAA is PSMA.

286. The TBM of embodiment 278, wherein the TAA is ROR1.

287. The TBM of embodiment 278, wherein the TAA is FLT3.

288. The TBM of embodiment 278, wherein the TAA is TAAG72.

289. The TBM of embodiment 278, wherein the TAA is FAP.

290. The TBM of embodiment 278, wherein the TAA is CD38.

291. The TBM of embodiment 278, wherein the TAA is CD44v6.

292. The TBM of embodiment 278, wherein the TAA is CEA.

293. The TBM of embodiment 278, wherein the TAA is EPCAM.

294. The TBM of embodiment 278, wherein the TAA is PRSS21.

295. The TBM of embodiment 278, wherein the TAA is B7H3.

296. The TBM of embodiment 278, wherein the TAA is KIT.

297. The TBM of embodiment 278, wherein the TAA is IL-13Ra2.

298. The TBM of embodiment 278, wherein the TAA is CD30.

299. The TBM of embodiment 278, wherein the TAA is GD3.

300. The TBM of embodiment 278, wherein the TAA is CD171.

301. The TBM of 278, wherein the TAA is IL-11Ra.

302. The TBM of embodiment 278, wherein the TAA is PSCA.

303. The TBM of embodiment 278, wherein the TAA is VEGFR2.

304. The TBM of embodiment 278, wherein the TAA is LewisY.

305. The TBM of embodiment 278, wherein the TAA is CD24.

306. The TBM of embodiment 278, wherein the TAA is PDGFR-beta.

307. The TBM of embodiment 278, wherein the TAA is SSEA-4.

308. The TBM of embodiment 278, wherein the TAA is CD20.

309. The TBM of embodiment 278, wherein the TAA is folate receptor alpha.

310. The TBM of embodiment 278, wherein the TAA is ERBB2.

311. The TBM of embodiment 278, wherein the TAA is MUC1.

312. The TBM of embodiment 278, wherein the TAA is EGFR.

313. The TBM of embodiment 278, wherein the TAA is NCAM.

314. The TBM of embodiment 278, wherein the TAA is ephrin B2

315. The TBM of embodiment 278, wherein the TAA is IGF-I receptor.

316. The TBM of embodiment 278, wherein the TAA is CAIX.

317. The TBM of embodiment 278, wherein the TAA is LMP2.

318. The TBM of embodiment 278, wherein the TAA is gp100.

319. The TBM of embodiment 278, wherein the TAA is tyrosinase.

320. The TBM of embodiment 278, wherein the TAA is ephA2.

321. The TBM of embodiment 278, wherein the TAA is mesothelin.

322. The TBM of embodiment 278, wherein the TAA is ALK.

323. The TBM of embodiment 278, wherein the TAA is CD19.

324. The TBM of embodiment 278, wherein the TAA is CD97.

325. The TBM of embodiment 278, wherein the TAA is CLDN6.

326. The TBM of embodiment 278, wherein the TAA is EGFRvIII.

327. The TBM of embodiment 278, wherein the TAA is folate receptor beta.

328. The TBM of embodiment 278, wherein the TAA is GloboH.

329. The TBM of embodiment 278, wherein the TAA is GPRC5D.

330. The TBM of embodiment 278, wherein the TAA is HMWMAA.

331. The TBM of embodiment 278, wherein the TAA is LRP6.

332. The TBM of embodiment 278, wherein the TAA is NY-BR-1.

333. The TBM of embodiment 278, wherein the TAA is PLAC1.

334. The TBM of embodiment 278, wherein the TAA is polysialic acid.

335. The TBM of embodiment 278, wherein the TAA is TEM1/CD248.

336. The TBM of embodiment 278, wherein the TAA is TSHR.

337. The TBM of embodiment 278, wherein the TAA is CD19.

338. The TBM of embodiment 278, wherein the TAA is Her2.

339. The TBM of embodiment of any one of embodiments 31 to 338, wherein ABM1 is an antibody, an antibody fragment, an scFv, a dsFv, a Fv, a Fab, an scFab, a (Fab′)2, a single domain antibody (SDAB), a VH or VL domain, or a camelid VHH domain.

340. The TBM of embodiment 339, wherein ABM1 is an scFv.

341. The TBM of embodiment of embodiment 339, wherein ABM1 is a Fab.

342. The TBM of any one of embodiments 1 to 341, wherein ABM2 is an antibody, an antibody fragment, an scFv, a dsFv, a Fv, a Fab, an scFab, a (Fab′)2, a single domain antibody (SDAB), a VH or VL domain, or a camelid VHH domain.

343. The TBM of embodiment 342, wherein ABM2 is an scFv.

344. The TBM of embodiment 343, wherein the scFv comprises the amino acid sequence of the scFV binding domain designated as CD3-21.

345. The TBM of any of embodiment 342, wherein ABM2 is a Fab.

346. The TBM of any one of embodiments 1 to 345, except when depending from any one of embodiments 277 to 338, wherein ABM3 is an antibody, an antibody fragment, an scFv, a dsFv, a Fv, a Fab, an scFab, a (Fab′)2, a single domain antibody (SDAB), a VH or VL domain, or a camelid VHH domain.

347. The TBM of embodiment 346, wherein ABM3 is an scFv.

348. The TBM of embodiment 347, wherein the TAA is CD19 and the scFv comprises the amino acid sequence of CD19-scFv1 as set forth in Table 13.

349. The TBM of embodiment 347, wherein the TAA is CD19 and the scFv comprises the amino acid sequence of CD19-scFv2 as set forth in Table 13.

350. The TBM of embodiment 347, wherein the TAA is CD19 and the scFv comprises the amino acid sequence of CD19-scFv3 as set forth in Table 13.

351. The TBM of embodiment 347, wherein the TAA is CD19 and the scFv comprises the amino acid sequence of CD19-scFv4 as set forth in Table 13.

352. The TBM of embodiment 347, wherein the TAA is CD19 and the scFv comprises the amino acid sequence of CD19-scFv5 as set forth in Table 13.

353. The TBM of embodiment 347, wherein the TAA is CD19 and the scFv comprises the amino acid sequence of CD19-scFv6 as set forth in Table 13.

354. The TBM of embodiment 347, wherein the TAA is CD19 and the scFv comprises the amino acid sequence of CD19-scFv7 as set forth in Table 13.

355. The TBM of embodiment 347, wherein the TAA is CD19 and the scFv comprises the amino acid sequence of CD19-scFv8 as set forth in Table 13.

356. The TBM of embodiment 347, wherein the TAA is CD19 and the scFv comprises the amino acid sequence of CD19-scFv9 as set forth in Table 13.

357. The TBM of embodiment 347, wherein the TAA is CD19 and the scFv comprises the amino acid sequence of CD19-scFv10 as set forth in Table 13.

358. The TBM of embodiment 347, wherein the TAA is CD19 and the scFv comprises the amino acid sequence of CD19-scFv11 as set forth in Table 13.

359. The TBM of embodiment 347, wherein the TAA is CD19 and the scFv comprises the amino acid sequence of CD19-scFv12 as set forth in Table 13.

360. The TBM of embodiment 347, wherein the TAA is BCMA and the scFv comprises the amino acid sequence of scFv corresponding to BCMA-1 as set forth in Table 12A.

361. The TBM of embodiment 347, wherein the TAA is BCMA and the scFv comprises the amino acid sequence of scFv corresponding to BCMA-2 as set forth in Table 12A.

362. The TBM of embodiment 347, wherein the TAA is BCMA and the scFv comprises the amino acid sequence of scFv corresponding to BCMA-3 as set forth in Table 12A.

363. The TBM of embodiment 347, wherein the TAA is BCMA and the scFv comprises the amino acid sequence of scFv corresponding to BCMA-4 as set forth in Table 12A.

364. The TBM of embodiment 347, wherein the TAA is BCMA and the scFv comprises the amino acid sequence of scFv corresponding to BCMA-5 as set forth in Table 12A.

365. The TBM of embodiment 347, wherein the TAA is BCMA and the scFv comprises the amino acid sequence of scFv corresponding to BCMA-6 as set forth in Table 12A.

366. The TBM of embodiment 347, wherein the TAA is BCMA and the scFv comprises the amino acid sequence of scFv corresponding to BCMA-7 as set forth in Table 12A.

367. The TBM of embodiment 347, wherein the TAA is BCMA and the scFv comprises the amino acid sequence of scFv corresponding to BCMA-8 as set forth in Table 12A.

368. The TBM of embodiment 347, wherein the TAA is BCMA and the scFv comprises the amino acid sequence of scFv corresponding to BCMA-9 as set forth in Table 12A.

369. The TBM of embodiment 347, wherein the TAA is BCMA and the scFv comprises the amino acid sequence of scFv corresponding to BCMA-10 as set forth in Table 12A.

370. The TBM of embodiment 347, wherein the TAA is BCMA and the scFv comprises the amino acid sequence of scFv corresponding to BCMA-11 as set forth in Table 12A.

371. The TBM of embodiment 347, wherein the TAA is BCMA and the scFv comprises the amino acid sequence of scFv corresponding to BCMA-12 as set forth in Table 12A.

372. The TBM of embodiment 347, wherein the TAA is BCMA and the scFv comprises the amino acid sequence of scFv corresponding to BCMA-13 as set forth in Table 12A.

373. The TBM of embodiment 347, wherein the TAA is BCMA and the scFv comprises the amino acid sequence of scFv corresponding to BCMA-14 as set forth in Table 12A.

374. The TBM of embodiment 347, wherein the TAA is BCMA and the scFv comprises the amino acid sequence of scFv corresponding to BCMA-15 as set forth in Table 12A.

375. The TBM of embodiment 347, wherein the TAA is BCMA and the scFv comprises the amino acid sequence of scFv corresponding to BCMA-16 as set forth in Table 12A.

376. The TBM of embodiment 347, wherein the TAA is BCMA and the scFv comprises the amino acid sequence of scFv corresponding to BCMA-17 as set forth in Table 12A.

377. The TBM of embodiment 347, wherein the TAA is BCMA and the scFv comprises the amino acid sequence of scFv corresponding to BCMA-18 as set forth in Table 12A.

378. The TBM of embodiment 347, wherein the TAA is BCMA and the scFv comprises the amino acid sequence of scFv corresponding to BCMA-19 as set forth in Table 12A.

379. The TBM of embodiment 347, wherein the TAA is BCMA and the scFv comprises the amino acid sequence of scFv corresponding to BCMA-20 as set forth in Table 12A.

380. The TBM of embodiment 347, wherein the TAA is BCMA and the scFv comprises the amino acid sequence of scFv corresponding to BCMA-21 as set forth in Table 12A.

381. The TBM of embodiment 347, wherein the TAA is BCMA and the scFv comprises the amino acid sequence of scFv corresponding to BCMA-22 as set forth in Table 12A.

382. The TBM of embodiment 347, wherein the TAA is BCMA and the scFv comprises the amino acid sequence of scFv corresponding to BCMA-23 as set forth in Table 12A.

383. The TBM of embodiment 347, wherein the TAA is BCMA and the scFv comprises the amino acid sequence of scFv corresponding to BCMA-24 as set forth in Table 12A.

384. The TBM of embodiment 347, wherein the TAA is BCMA and the scFv comprises the amino acid sequence of scFv corresponding to BCMA-25 as set forth in Table 12A.

385. The TBM of embodiment 347, wherein the TAA is BCMA and the scFv comprises the amino acid sequence of scFv corresponding to BCMA-26 as set forth in Table 12A.

386. The TBM of embodiment 347, wherein the TAA is BCMA and the scFv comprises the amino acid sequence of scFv corresponding to BCMA-27 as set forth in Table 12A.

387. The TBM of embodiment 347, wherein the TAA is BCMA and the scFv comprises the amino acid sequence of scFv corresponding to BCMA-28 as set forth in Table 12A.

388. The TBM of embodiment 347, wherein the TAA is BCMA and the scFv comprises the amino acid sequence of scFv corresponding to BCMA-29 as set forth in Table 12A.

389. The TBM of embodiment 347, wherein the TAA is BCMA and the scFv comprises the amino acid sequence of scFv corresponding to BCMA-30 as set forth in Table 12A.

390. The TBM of embodiment 347, wherein the TAA is BCMA and the scFv comprises the amino acid sequence of scFv corresponding to BCMA-31 as set forth in Table 12A.

391. The TBM of embodiment 347, wherein the TAA is BCMA and the scFv comprises the amino acid sequence of scFv corresponding to BCMA-32 as set forth in Table 12A.

392. The TBM of embodiment 347, wherein the TAA is BCMA and the scFv comprises the amino acid sequence of scFv corresponding to BCMA-33 as set forth in Table 12A.

393. The TBM of embodiment 347, wherein the TAA is BCMA and the scFv comprises the amino acid sequence of scFv corresponding to BCMA-34 as set forth in Table 12A.

394. The TBM of embodiment 347, wherein the TAA is BCMA and the scFv comprises the amino acid sequence of scFv corresponding to BCMA-35 as set forth in Table 12A.

395. The TBM of embodiment 347, wherein the TAA is BCMA and the scFv comprises the amino acid sequence of scFv corresponding to BCMA-36 as set forth in Table 12A.

396. The TBM of embodiment 347, wherein the TAA is BCMA and the scFv comprises the amino acid sequence of scFv corresponding to BCMA-37 as set forth in Table 12A.

397. The TBM of embodiment 347, wherein the TAA is BCMA and the scFv comprises the amino acid sequence of scFv corresponding to BCMA-38 as set forth in Table 12A.

398. The TBM of embodiment 347, wherein the TAA is BCMA and the scFv comprises the amino acid sequence of scFv corresponding to BCMA-39 as set forth in Table 12A.

399. The TBM of embodiment 347, wherein the TAA is BCMA and the scFv comprises the amino acid sequence of scFv corresponding to BCMA-40 as set forth in Table 12A.

400. The TBM of embodiment 346, wherein ABM3 is a Fab. 401. The TBM of any one of embodiments 1 to 400, which comprises:
(a) a first monomer or half antibody comprising:
   (i) a first chain comprising a first variant Fc region and a first heavy chain variable domain;
   (ii) a first scFv domain; and
(b) a second monomer or half antibody comprising:
   (i) a second chain comprising a second variant Fc region and first heavy chain variable domain;
   (ii) a second scFv domain; and
(c) a third chain comprising a light chain constant domain and a light chain variable domain; wherein:
   (1) the first and second variant Fc regions form a heterodimer,
   (2) the first heavy chain variable domain and the light chain variable domain form ABM1, provided that TBM1 is not a CD58 moiety,
   (3) the first scFv domain forms ABM2, and
   (4) the second scFv domain forms ABM3.

402. The TBM of any one of embodiments 1 to 400, which comprises:
(a) a first monomer or half antibody comprising:
   (i) a first chain comprising a first variant Fc region and a first heavy chain variable domain;
   (ii) a first scFv domain; and
(b) a second monomer or half antibody comprising:
   (i) a second chain comprising a second variant Fc region and first heavy chain variable domain;
   (ii) a second scFv domain; and
(c) a third chain comprising a light chain constant domain and a light chain variable domain; wherein:
   (1) the first and second variant Fc regions form a heterodimer,
   (2) the first heavy chain variable domain and the light chain variable domain form ABM1, provided that TBM1 is not a CD58 moiety,
   (3) the first scFv domain forms ABM3, and
   (4) the second scFv domain forms ABM2.

403. The TBM of any one of embodiments 1 to 400, which comprises:
(a) a first monomer or half antibody comprising:
   (i) a first chain comprising a first variant Fc region and a first heavy chain variable domain;
   (ii) a first scFv domain or CD58 moiety; and
(b) a second monomer or half antibody comprising:
   (i) a second chain comprising a second variant Fc region and first heavy chain variable domain;
   (ii) a second scFv domain; and
(c) a third chain comprising a light chain constant domain and a light chain variable domain; wherein
   (1) the first and second variant Fc regions form a heterodimer,
   (2) the first heavy chain variable domain and the light chain variable domain form ABM2,
   (3) the first scFv domain or CD58 moiety forms ABM1, and
   (4) the second scFv domain forms ABM3.

404. The TBM of any one of embodiments 1 to 400, which comprises:
(a) a first monomer or half antibody comprising:
   (i) a first chain comprising a first variant Fc region and a first heavy chain variable domain;
   (ii) a first scFv domain; and
(b) a second monomer or half antibody comprising:
   (i) a second chain comprising a second variant Fc region and first heavy chain variable domain;
   (ii) a second scFv domain or a CD58 moiety; and
(c) a third chain comprising a light chain constant domain and a light chain variable domain; wherein
   (1) the first and second variant Fc regions form a heterodimer,
   (2) the first heavy chain variable domain and the light chain variable domain form ABM2,
   (3) the first scFv domain forms ABM3, and
   (4) the second scFv domain or CD58 moiety forms ABM1.

405. The TBM of any one of embodiments 1 to 400, which comprises:
(a) a first monomer or half antibody comprising:
   (i) a first chain comprising a first variant Fc region and a first heavy chain variable domain;
   (ii) a first scFv domain; and
(b) a second monomer or half antibody comprising:
   (i) a second chain comprising a second variant Fc region and first heavy chain variable domain;
   (ii) a second scFv domain or a CD58 moiety; and
a third chain comprising a light chain constant domain and a light chain variable domain; wherein:
   (1) the first and second variant Fc regions form a heterodimer,
   (2) the first heavy chain variable domain and the light chain variable domain form ABM3,
   (3) the first scFv domain forms ABM2, and
   (4) the second scFv domain or CD58 moiety forms ABM1.

406. The TBM of any one of embodiments 1 to 400, which comprises:
(a) a first monomer or half antibody comprising:
   (i) a first chain comprising a first variant Fc region and a first heavy chain variable domain;
   (ii) a first scFv domain or CD58 moiety; and
(b) a second monomer or half antibody comprising:
   (i) a second chain comprising a second variant Fc region and first heavy chain variable domain;

(ii) a second scFv domain; and
(c) a third chain comprising a light chain constant domain and a light chain variable domain; wherein
   (1) the first and second variant Fc regions form a heterodimer,
   (2) the first heavy chain variable domain and the light chain variable domain form ABM3,
   (3) the first scFv domain or CD58 moiety forms ABM1, and
   (4) the second scFv domain forms ABM2.

407. The TBM of any one of embodiments 401 to 406, wherein the first and second variant Fc regions comprise the amino acid substitutions S364K/E357Q:L368D/K370S.

408. The TBM of any one of embodiments 401 to 406, wherein the first and second variant Fc regions comprise the amino acid substitutions L368D/K370S:S364.

409. The TBM of any one of embodiments 401 to 406, wherein the first and second variant Fc regions comprise the amino acid substitutions L368E/K370S:S364K.

410. The TBM of any one of embodiments 401 to 406, wherein the first and second variant Fc regions comprise the amino acid substitutions T411T/E360E/Q362E:D401K.

411. The TBM of any one of embodiments 401 to 406, wherein the first and second variant Fc regions comprise the amino acid substitutions L368D 370S:S364/E357L.

412. The TBM of any one of embodiments 401 to 406, wherein the first and second variant Fc regions comprise the amino acid substitutions 370S:S364K/E357Q.

413. The TBM of any one of embodiments 401 to 406, wherein the first and second variant Fc regions comprise the amino acid substitutions of any of the steric variants listed in FIG. 4 of WO 2014/110601 (reproduced in Table 2).

414. The TBM of any one of embodiments 401 to 406, wherein the first and second variant Fc regions comprise the amino acid substitutions of any of the variants listed in FIG. 5 of WO 2014/110601 (reproduced in Table 2).

415. The TBM of any one of embodiments 401 to 406, wherein the first and second variant Fc regions comprise the amino acid substitutions of any of the variants listed in FIG. 6 of WO 2014/110601 (reproduced in Table 2).

416. The TBM of any one of embodiments 401 to 415, wherein at least one of the monomers or half antibodies further comprises pI variant substitutions.

417. The TBM of embodiment 416 wherein said pI variant substitutions are selected from Table 2.

418. The TBM of embodiment 417, wherein the pI variant substitutions comprise the substitutions present in pI_ISO(−).

419. The TBM of embodiment 417, wherein the pI variant substitutions comprise the substitutions present in pI_(−)_isosteric_A.

420. The TBM of embodiment 417, wherein the pI variant substitutions comprise the substitutions present in pI_(−)_isosteric_B.

421. The TBM of embodiment 417, wherein the pI variant substitutions comprise the substitutions present in PI_ISO(+RR).

422. The TBM of embodiment 417, wherein the pI variant substitutions comprise the substitutions present in pI_ISO(+).

423. The TBM of embodiment 417, wherein the pI variant substitutions comprise the substitutions present in pI_(+)_isosteric_A.

424. The TBM of embodiment 417, wherein the pI variant substitutions comprise the substitutions present in pI_(+)_isosteric_B.

425. The TBM of embodiment 417, wherein the pI variant substitutions comprise the substitutions present in pI_(+)_isosteric_E269Q/E272Q.

426. The TBM of embodiment 417, wherein the pI variant substitutions comprise the substitutions present in pI_(+)_isosteric_E269Q/E283Q.

427. The TBM of embodiment 417, wherein the pI variant substitutions comprise the substitutions present in pI_(+)_isosteric_E2720/E283Q.

428. The TBM of embodiment 417, wherein the pI variant substitutions comprise the substitutions present in pI_(+)_isosteric_E269Q.

429. The TBM of embodiment any of embodiments 401 to 428, wherein said first and second scFv domains are covalently attached to the C-terminus of said first and second chains, respectively.

430. The TBM of embodiment any of embodiments 401 to 428, wherein said first and second scFv domains are covalently attached to the N-terminus of said first and second chains, respectively.

431. The TBM of embodiment any of embodiments 401 to 428, wherein each of the scFv domains is attached between said Fc region and the CH domain of said chain.

432. The TBM of embodiment any of embodiments 401 to 431, wherein the scFv domains are covalently attached using one or more domain linkers.

433. The TBM of embodiment any of embodiments 401 to 432, wherein the scFv domains comprise at least one scFv linker.

434. The TBM of embodiment 433, wherein at least one scFv linker is charged.

435. The TBM of embodiment 434, wherein the charged linker is selected from L1 through L54.

436. The TBM of embodiment any of embodiments 401 to 435, wherein the first and/or second Fc region further comprises one or more amino acid substitution(s) selected from 434A, 434S, 428L, 308F, 2591, 428L/434S, 2591/308F, 4361/428L, 4361 or V/434S, 436V/428L, 252Y, 252Y/254T/256E, 2591/308F/428L, 236A, 239D, 239E, 332E, 332D, 239D/332E, 267D, 267E, 328F, 267E/328F, 236A/332E, 239D/332E/330Y, 239D, 332E/330L, 236R, 328R, 236R/328R, 236N/267E, 243L, 298A and 299T.

437. The TBM of embodiment any of embodiments 401 to 435, wherein the first and/or second Fc region further comprises one or more amino acid substitution comprises the amino acid substitution 434A, 434S or 434V.

438. The TBM of embodiment 437, wherein the first and/or second Fc region further comprises one or more amino acid substitution comprises the amino acid substitution 428L.

439. The TBM of any one of embodiments 437 to 438, wherein the first and/or second Fc region further comprises one or more amino acid substitution comprises the amino acid substitution 308F.

440. The TBM of any one of embodiments 437 to 439, wherein the first and/or second Fc region further comprises one or more amino acid substitution comprises the amino acid substitution 2591.

441. The TBM of any one of embodiments 437 to 440, wherein the first and/or second Fc region further comprises one or more amino acid substitution comprises the amino acid substitution 4361.

442. The TBM of any one of embodiments 437 to 441, wherein the first and/or second Fc region further comprises one or more amino acid substitution comprises the amino acid substitution 252Y.

443. The TBM of any one of embodiments 437 to 442, wherein the first and/or second Fc region further comprises one or more amino acid substitution comprises the amino acid substitution 254T.

444. The TBM of any one of embodiments 437 to 443, wherein the first and/or second Fc region further comprises one or more amino acid substitution comprises the amino acid substitution 256E.

445. The TBM of any one of embodiments 437 to 444, wherein the first and/or second Fc region further comprises one or more amino acid substitution comprises the amino acid substitution 239D or 239E.

446. The TBM of any one of embodiments 437 to 445, wherein the first and/or second Fc region further comprises one or more amino acid substitution comprises the amino acid substitution 332E or 332D.

447. The TBM of any one of embodiments 437 to 446, wherein the first and/or second Fc region further comprises one or more amino acid substitution comprises the amino acid substitution 267D or 267E.

448. The TBM of any one of embodiments 437 to 447, wherein the first and/or second Fc region further comprises one or more amino acid substitution comprises the amino acid substitution 330L.

449. The TBM of any one of embodiments 437 to 448, wherein the first and/or second Fc region further comprises one or more amino acid substitution comprises the amino acid substitution 236R or 236N.

450. The TBM of any one of embodiments 437 to 449, wherein the first and/or second Fc region further comprises one or more amino acid substitution comprises the amino acid substitution 328R.

451. The TBM of any one of embodiments 437 to 450, wherein the first and/or second Fc region further comprises one or more amino acid substitution comprises the amino acid substitution 243L.

452. The TBM of any one of embodiments 437 to 451, wherein the first and/or second Fc region further comprises one or more amino acid substitution comprises the amino acid substitution 298A.

453. The TBM of any one of embodiments 437 to 452, wherein the first and/or second Fc region further comprises one or more amino acid substitution comprises the amino acid substitution 299T.

454. The TBM of embodiment 1, which comprises the amino acid sequences of trispecific AB1 set forth in Table 15.

455. The TBM of embodiment 1, which comprises the amino acid sequences of AB2-2 set forth in Table 20.

456. The TBM of embodiment 1, which comprises the amino acid sequences of AB2-3 set forth in Table 20.

457. The TBM of embodiment 1, which comprises the amino acid sequences of AB3-2 set forth in Table 21.

458. The TBM of embodiment 1, which comprises the amino acid sequences of AB3-3 set forth in Table 21.

459. The TBM of embodiment 1, which comprises the amino acid sequences of AB3-4 set forth in Table 21.

460. The TBM of embodiment 1, which comprises the amino acid sequences of AB3-5 set forth in Table 21.

461. The TBM of embodiment 1, which comprises the amino acid sequences of AB4-2 set forth in Table 22.

462. The TBM of embodiment 1, which comprises the amino acid sequences of AB4-3 set forth in Table 22.

463. The TBM of embodiment 1, which comprises the amino acid sequences of the TBM set forth in Table 23A.

464. The TBM of embodiment 1, which comprises the amino acid sequences of the TBM set forth in Table 23B.

465. The TBM of embodiment 1, which comprises the amino acid sequences of the TBM set forth in Table 24.

466. The TBM of embodiment 1, which comprises the amino acid sequences of the TBM set forth in Table 25.

467. The TBM of embodiment 1, which comprises the amino acid sequences of the TBM set forth in Table 26.

468. The TBM of any one of embodiments 1 to 467, which has been recombinantly produced, optionally in a mammalian host cell, which is optionally selected from Vero cells, HeLa cells, COS cells, CHO cells, HEK293 cells, BHK cells and MDCKII cells.

469. A conjugate comprising the TBM of any one of embodiments 1 to 468 and an agent.

470. The conjugate of embodiment 469, wherein the agent is a therapeutic agent, a diagnostic agent, a masking moiety, a cleavable moiety, or any combination thereof.

471. The conjugate of embodiment 470, wherein the agent is any of the agents described in Section 4.9.

472. The conjugate of embodiment 470, wherein the agent is any of the agents described in Section 4.9.1.

473. The conjugate of any one of embodiments 469 to 472, wherein the TBM is conjugated to a radionuclide.

474. The conjugate of any one of embodiments 469 to 472, wherein the TBM is conjugated to an alkylating agent.

475. The conjugate of any one of embodiments 469, wherein the TBM is conjugated to a topoisomerase inhibitor, which is optionally a topoisomerase I inhibitor or a topoisomerase II inhibitor.

476. The conjugate of any one of embodiments 469 to 472, wherein the TBM is conjugated to a DNA damaging agent.

477. The conjugate of any one of embodiments 469 to 472, wherein the TBM is conjugated to a DNA intercalating agent, optionally a groove binding agent such as a minor groove binding agent.

478. The conjugate of any one of embodiments 469 to 472, wherein the TBM is conjugated to a RNA/DNA antimetabolite.

479. The conjugate of any one of embodiments 469 to 472, wherein the TBM is conjugated to a kinase inhibitor.

480. The conjugate of any one of embodiments 469 to 472, wherein the TBM is conjugated to a protein synthesis inhibitor.

481. The conjugate of any one of embodiments 469 to 472, wherein the TBM is conjugated to a histone deacetylase (HDAC) inhibitor.

482. The conjugate of any one of embodiments 469 to 472, wherein the TBM is conjugated to a mitochondrial. Inhibitor, which is optionally an inhibitor of a phosphoryl transfer reaction in mitochondria.

483. The conjugate of any one of embodiments 469 to 472, wherein the TBM is conjugated to an antimitotic agent.

484. The conjugate of any one of embodiments 469 to 472, wherein the TBM is conjugated to a maytansinoid.

485. The conjugate of any one of embodiments 469 to 472, wherein the TBM is conjugated to a kinesin inhibitor.

486. The conjugate of any one of embodiments 469 to 472, wherein the TBM is conjugated to a kinesin-like protein KIF11 inhibitor.

487. The conjugate of any one of embodiments 469 to 472, wherein the TBM is conjugated to a V-ATPase (vacuolar-type H+-ATPase) inhibitor.

488. The conjugate of any one of embodiments 469 to 472, wherein the TBM is conjugated to a pro-apoptotic agent.

489. The conjugate of any one of embodiments 469 to 472, wherein the TBM is conjugated to a Bcl2 (B-cell lymphoma 2) inhibitor.

490. The conjugate of any one of embodiments 469 to 472, wherein the TBM is conjugated to an MCL1 (myeloid cell leukemia 1) inhibitor.

491. The conjugate of any one of embodiments 469 to 472, wherein the TBM is conjugated to a HSP90 (heat shock protein 90) inhibitor.

492. The conjugate of any one of embodiments 469 to 472, wherein the TBM is conjugated to an IAP (inhibitor of apoptosis) inhibitor.

493. The conjugate of any one of embodiments 469 to 472, wherein the TBM is conjugated to an mTOR (mechanistic target of rapamycin) inhibitor.

494. The conjugate of any one of embodiments 469 to 472, wherein the TBM is conjugated to a microtubule stabilizer.

495. The conjugate of any one of embodiments 469 to 472, wherein the TBM is conjugated to a microtubule destabilizer.

496. The conjugate of any one of embodiments 469 to 472, wherein the TBM is conjugated to an auristatin.

497. The conjugate of any one of embodiments 469 to 472, wherein the TBM is conjugated to a dolastatin.

498. The conjugate of any one of embodiments 469 to 472, wherein the TBM is conjugated to a MetAP (methionine aminopeptidase).

499. The conjugate of any one of embodiments 469 to 472, wherein the TBM is conjugated to a CRM1 (chromosomal maintenance 1) inhibitor.

500. The conjugate of any one of embodiments 469 to 472, wherein the TBM is conjugated to a DPPIV (dipeptidyl peptidase IV) inhibitor.

501. The conjugate of any one of embodiments 469 to 472, wherein the TBM is conjugated to a proteasome inhibitor.

502. The conjugate of any one of embodiments 469 to 472, wherein the TBM is conjugated to a protein synthesis inhibitor.

503. The conjugate of any one of embodiments 469 to 472, wherein the TBM is conjugated to a CDK2 (cyclin-dependent kinase 2) inhibitor.

504. The conjugate of any one of embodiments 469 to 472, wherein the TBM is conjugated to a CDK9 (cyclin-dependent kinase 9) inhibitor.

505. The conjugate of any one of embodiments 469 to 472, wherein the TBM is conjugated to a RNA polymerase inhibitor.

506. The conjugate of any one of embodiments 469 to 472, wherein the TBM is conjugated to a DHFR (dihydrofolate reductase) inhibitor.

507. The conjugate of any one of embodiments 469 to 506, wherein the agent is attached to the TBM with a linker, which is optionally a cleavable linker or a non-cleavabe linker, e.g., a linker as described in Section 4.9.2.

508. A preparation of TBMs comprising a plurality of TBM molecules according to any one of embodiments 1 to 468 or a plurality of conjugate molecules according to any one of embodiments 469 to 507, optionally wherein the plurality comprises at least 100, at least 1,000, at least 10,000, or at least 100,000 TBM molecules or conjugate molecules.

509. The preparation of embodiment 508, wherein at least 50% of the trispecific molecules in the preparation have the same primary amino acid sequence.

510. The preparation of embodiment 508, wherein at least 60% of the trispecific molecules in the preparation have the same primary amino acid sequence.

511. The preparation of embodiment 508, wherein at least 70% of the trispecific molecules in the preparation have the same primary amino acid sequence.

512. The preparation of embodiment 508, wherein at least 80% of the trispecific molecules in the preparation have the same primary amino acid sequence.

513. The preparation of embodiment 508, wherein at least 90% of the trispecific molecules in the preparation have the same primary amino acid sequence.

514. The preparation of embodiment 508, wherein at least 95% of the trispecific molecules in the preparation have the same primary amino acid sequence.

515. The preparation of embodiment 508, wherein at least 97% of the trispecific molecules in the preparation have the same primary amino acid sequence.

516. The preparation of embodiment 508, wherein at least 98% of the trispecific molecules in the preparation have the same primary amino acid sequence.

517. The preparation of embodiment 508, wherein at least 99% of the trispecific molecules in the preparation have the same primary amino acid sequence.

518. The preparation of embodiment 508, wherein 50% to 95% of the trispecific molecules in the preparation have the same primary amino acid sequence.

519. The preparation of embodiment 508, wherein 50% to 80% of the trispecific molecules in the preparation have the same primary amino acid sequence.

520. The preparation of embodiment 508, wherein 50% to 70% of the trispecific molecules in the preparation have the same primary amino acid sequence.

521. The preparation of embodiment 508, wherein 60% to 95% of the trispecific molecules in the preparation have the same primary amino acid sequence.

522. The preparation of embodiment 508, wherein 60% to 80% of the trispecific molecules in the preparation have the same primary amino acid sequence.

523. The preparation of embodiment 508, wherein 60% to 70% of the trispecific molecules in the preparation have the same primary amino acid sequence.

524. The preparation of embodiment 508, wherein 70% to 95% of the trispecific molecules in the preparation have the same primary amino acid sequence.

525. The preparation of embodiment 508, wherein 70% to 80% of the trispecific molecules in the preparation have the same primary amino acid sequence.

526. The preparation of embodiment 508, wherein 80% to 95% of the trispecific molecules in the preparation have the same primary amino acid sequence.

527. The preparation of embodiment 508, wherein 95% to 99% of the trispecific molecules in the preparation have the same primary amino acid sequence.

528. The preparation of any one of embodiments 508 to 527, wherein at least 50% of the trispecific molecules in the preparation have the same interchain crosslinks.

529. The preparation of any one of embodiments 508 to 527, wherein at least 60% of the trispecific molecules in the preparation have the same interchain crosslinks.

530. The preparation of any one of embodiments 508 to 527, wherein at least 70% of the trispecific molecules in the preparation have the same interchain crosslinks.

531. The preparation of any one of embodiments 508 to 527, wherein at least 80% of the trispecific molecules in the preparation have the same interchain crosslinks.

532. The preparation of any one of embodiments 508 to 527, wherein at least 90% of the trispecific molecules in the preparation have the same interchain crosslinks.

533. The preparation of any one of embodiments 508 to 527, wherein at least 95% of the trispecific molecules in the preparation have the same interchain crosslinks.

534. The preparation of any one of embodiments 508 to 527, wherein at least 97% of the trispecific molecules in the preparation have the same interchain crosslinks.

535. The preparation of any one of embodiments 508 to 527, wherein at least 98% of the trispecific molecules in the preparation have the same interchain crosslinks.

536. The preparation of any one of embodiments 508 to 527, wherein at least 99% of the trispecific molecules in the preparation have the same interchain crosslinks.

537. The preparation of any one of embodiments 508 to 527, wherein 50% to 95% of the trispecific molecules in the preparation have the same interchain crosslinks.

538. The preparation of any one of embodiments 508 to 527, wherein 50% to 80% of the trispecific molecules in the preparation have the same interchain crosslinks.

539. The preparation of any one of embodiments 508 to 527, wherein 50% to 70% of the trispecific molecules in the preparation have the same interchain crosslinks.

540. The preparation of any one of embodiments 508 to 527, wherein 60% to 95% of the trispecific molecules in the preparation have the same interchain crosslinks.

541. The preparation of any one of embodiments 508 to 527, wherein 60% to 80% of the trispecific molecules in the preparation have the same interchain crosslinks.

542. The preparation of any one of embodiments 508 to 527, wherein 60% to 70% of the trispecific molecules in the preparation have the same interchain crosslinks.

543. The preparation of any one of embodiments 508 to 527, wherein 70% to 95% of the trispecific molecules in the preparation have the same interchain crosslinks.

544. The preparation of any one of embodiments 508 to 527, wherein 70% to 80% of the trispecific molecules in the preparation have the same interchain crosslinks.

545. The preparation of any one of embodiments 508 to 527, wherein 80% to 95% of the trispecific molecules in the preparation have the same interchain crosslinks.

546. The preparation of any one of embodiments 508 to 527, wherein 95% to 99% of the trispecific molecules in the preparation have the same interchain crosslinks.

547. The preparation of any one of embodiments 508 to 546, wherein at least 50% of the trispecific molecules in the preparation have the same ABM1:ABM2:ABM3 ratio.

548. The preparation of any one of embodiments 508 to 546, wherein at least 60% of the trispecific molecules in the preparation have the same ABM1:ABM2:ABM3 ratio.

549. The preparation of any one of embodiments 508 to 546, wherein at least 70% of the trispecific molecules in the preparation have the same ABM1:ABM2:ABM3 ratio.

550. The preparation of any one of embodiments 508 to 546, wherein at least 80% of the trispecific molecules in the preparation have the same ABM1:ABM2:ABM3 ratio.

551. The preparation of any one of embodiments 508 to 546, wherein at least 90% of the trispecific molecules in the preparation have the same ABM1:ABM2:ABM3 ratio.

552. The preparation of any one of embodiments 508 to 546, wherein at least 95% of the trispecific molecules in the preparation have the same ABM1:ABM2:ABM3 ratio.

553. The preparation of any one of embodiments 508 to 546, wherein at least 97% of the trispecific molecules in the preparation have the same ABM1:ABM2:ABM3 ratio.

554. The preparation of any one of embodiments 508 to 546, wherein at least 98% of the trispecific molecules in the preparation have the same ABM1:ABM2:ABM3 ratio.

555. The preparation of any one of embodiments 508 to 546, wherein at least 99% of the trispecific molecules in the preparation have the same ABM1:ABM2:ABM3 ratio.

556. The preparation of any one of embodiments 508 to 546, wherein 50% to 95% of the trispecific molecules in the preparation have the same ABM1:ABM2:ABM3 ratio.

557. The preparation of any one of embodiments 508 to 546, wherein 50% to 80% of the trispecific molecules in the preparation have the same ABM1:ABM2:ABM3 ratio.

558. The preparation of any one of embodiments 508 to 546, wherein 50% to 70% of the trispecific molecules in the preparation have the same ABM1:ABM2:ABM3 ratio.

559. The preparation of any one of embodiments 508 to 546, wherein 60% to 95% of the trispecific molecules in the preparation have the same ABM1:ABM2:ABM3 ratio.

560. The preparation of any one of embodiments 508 to 546, wherein 60% to 80% of the trispecific molecules in the preparation have the same ABM1:ABM2:ABM3 ratio.

561. The preparation of any one of embodiments 508 to 546, wherein 60% to 70% of the trispecific molecules in the preparation have the same ABM1:ABM2:ABM3 ratio.

562. The preparation of any one of embodiments 508 to 546, wherein 70% to 95% of the trispecific molecules in the preparation have the same ABM1:ABM2:ABM3 ratio.

563. The preparation of any one of embodiments 508 to 546, wherein 70% to 80% of the trispecific molecules in the preparation have the same ABM1:ABM2:ABM3 ratio.

564. The preparation of any one of embodiments 508 to 546, wherein 80% to 95% of the trispecific molecules in the preparation have the same ABM1:ABM2:ABM3 ratio.

565. The preparation of any one of embodiments 508 to 546, wherein 95% to 99% of the trispecific molecules in the preparation have the same ABM1:ABM2:ABM3 ratio.

566. A pharmaceutical composition comprising the TBM of any one of embodiments 1 to 468, the conjugate of any one of embodiments 469 to 507, or the preparation of any one of embodiments 508 to 565, and an excipient.

567. A method of treating a subject with cancer, comprising administering to a subject suffering from cancer an effective amount of the TBM of any one of embodiments 1 to 468, the conjugate of any one of embodiments 469 to 507, the preparation of any one of embodiments 508 to 565, or the pharmaceutical composition of embodiment 566.

568. The method of embodiment 567, wherein the cancer is selected from HER2+ cancer, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

569. The method of any of embodiment 567 or embodiment 568, further comprising administering at least one further agent to the subject.

570. A cell engineered to express the TBM of any one of embodiments 1 to 468.

571. A cell transfected with one or more expression vectors comprising one or more nucleic acid sequences encoding the TBM of any one of embodiments 1 to 468 under the control of one or more promoters.

572. The cell of embodiment 570 or embodiment 571, wherein expression of the TBM is under the control of an inducible promoter.

573. The cell of any one of embodiments 570 to 572, wherein the TBM is produced in secretable form.

574. A method of producing a TBM, comprising:
(a) culturing the cell of any one of embodiments 570 to 573 in conditions under which the TBM is expressed; and
(b) recovering the TBM from the cell culture.

575. The TBM of embodiment 1, wherein ABM1 is
(a) an immunoglobulin scaffold-based ABM which is optionally an anti-CD2 antibody, an antibody fragment, an scFv, a dsFv, a Fv, a Fab, an scFab, a (Fab′)2, a single domain antibody (SDAB), a VH or VL domain, or a camelid VHH domain; or
(b) a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.

576. The TBM of embodiment 575, wherein ABM1 is an scFv.

577. The TBM of embodiment 575, wherein ABM1 is a Fab.

578. The TBM of embodiment 577, wherein the Fab is a Fab heterodimer.

579. The TBM of any one of embodiments 575 to 578, wherein ABM1 comprises any of the binding sequences set forth in Table 9.

580. The TBM of embodiment 579, wherein ABM1 comprises the heavy and light chain CDRs of CD2-1.

581. The TBM of embodiment 579, wherein ABM1 comprises the VH and VL sequences of CD2-1.

582. The TBM of embodiment 579, wherein ABM1 comprises the VH and VL sequences of hu1CD2-1.

583. The TBM of embodiment 579, wherein ABM1 comprises the VH and VL sequences of hu2CD2-1.

584. The TBM of embodiment 1, wherein ABM1 is a CD58 moiety.

585. The TBM of embodiment 584, wherein ABM1 comprises any of the binding sequences set forth in Table 10.

586. The TBM of embodiment 585, wherein ABM1 comprises the amino acid sequence designated as CD58-2.

587. The TBM of embodiment 585, wherein ABM1 comprises the amino acid sequence designated CD58-2.

588. The TBM of embodiment 585, wherein ABM1 comprises the amino acid sequence designated as CD58-4.

589. The TBM of embodiment 585, wherein ABM1 comprises the amino acid sequence designated as CD58-5.

590. The TBM of any one of embodiments 1 to 589, wherein the component of a human TCR complex is CD3.

591. The TBM of embodiment 590, wherein ABM2 is:
(a) an immunoglobulin scaffold-based ABM which is optionally an anti-CD3 antibody, an antibody fragment, an scFv, a dsFv, a Fv, a Fab, an scFab, a (Fab′)2, a single domain antibody (SDAB), a VH or VL domain, or a camelid VHH domain; or
(b) a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.

592. The TBM of embodiment 590 or embodiment 591, wherein ABM2 comprises any of the binding sequences set forth in any one of Tables 7A through 7D.

593. The TBM of embodiment 592, wherein ABM2 comprises the heavy and light chain CDRs (as defined by Kabat, Chothia or a combination thereof) of any one of the binding domains designated as CD3-1 through CD3-28.

594. The TBM of embodiment 593, wherein ABM2 comprises the heavy and light chain CDRs (as defined by Kabat, Chothia or a combination thereof) of the binding domain designated as CD3-1.

595. The TBM of embodiment 593, wherein ABM2 comprises the VH and/or VL sequences of the binding domain designated as CD3-1.

596. The TBM of embodiment 593, wherein ABM2 comprises the heavy and light chain CDRs (as defined by Kabat, Chothia or a combination thereof) of the binding domain designated as CD3-2.

597. The TBM of embodiment 593, wherein ABM2 comprises the VH and/or VL sequences of the binding domain designated as CD3-2.

598. The TBM of embodiment 593, wherein ABM2 comprises the heavy and light chain CDRs (as defined by Kabat, Chothia or a combination thereof) of the binding domain designated as CD3-3.

599. The TBM of embodiment 593, wherein ABM2 comprises the VH and/or VL sequences of the binding domain designated as CD3-3.

600. The TBM of embodiment 593, wherein ABM2 comprises the heavy and light chain CDRs (as defined by Kabat, Chothia or a combination thereof) of the binding domain designated as CD3-4.

601. The TBM of embodiment 593, wherein ABM2 comprises the VH and/or VL sequences of the binding domain designated as CD3-4.

602. The TBM of embodiment 593, wherein ABM2 comprises the heavy and light chain CDRs (as defined by Kabat, Chothia or a combination thereof) of the binding domain designated as CD3-5.

603. The TBM of embodiment 593, wherein ABM2 comprises the VH and/or VL sequences of the binding domain designated as CD3-5.

604. The TBM of embodiment 593, wherein ABM2 comprises the heavy and light chain CDRs (as defined by Kabat, Chothia or a combination thereof) of the binding domain designated as CD3-6.

605. The TBM of embodiment 593, wherein ABM2 comprises the VH and/or VL sequences of the binding domain designated as CD3-6.

606. The TBM of embodiment 593, wherein ABM2 comprises the heavy and light chain CDRs (as defined by Kabat, Chothia or a combination thereof) of the binding domain designated as CD3-7.

607. The TBM of embodiment 593, wherein ABM2 comprises the VH and/or VL sequences of the binding domain designated as CD3-7.

608. The TBM of embodiment 593, wherein ABM2 comprises the heavy and light chain CDRs (as defined by Kabat, Chothia or a combination thereof) of the binding domain designated as CD3-8.

609. The TBM of embodiment 593, wherein ABM2 comprises the VH and/or VL sequences of the binding domain designated as CD3-8.

610. The TBM of embodiment 593, wherein ABM2 comprises the heavy and light chain CDRs (as defined by Kabat, Chothia or a combination thereof) of the binding domain designated as CD3-9.

611. The TBM of embodiment 593, wherein ABM2 comprises the VH and/or VL sequences of the binding domain designated as CD3-9.

612. The TBM of embodiment 593, wherein ABM2 comprises the heavy and light chain CDRs (as defined by Kabat, Chothia or a combination thereof) of the binding domain designated as CD3-10.

613. The TBM of embodiment 593, wherein ABM2 comprises the VH and/or VL sequences of the binding domain designated as CD3-10.

614. The TBM of embodiment 593, wherein ABM2 comprises the heavy and light chain CDRs (as defined by Kabat, Chothia or a combination thereof) of the binding domain designated as CD3-11.

615. The TBM of embodiment 593, wherein ABM2 comprises the VH and/or VL sequences of the binding domain designated as CD3-11.

616. The TBM of embodiment 593, wherein ABM2 comprises the heavy and light chain CDRs (as defined by Kabat, Chothia or a combination thereof) of the binding domain designated as CD3-12.

617. The TBM of embodiment 593, wherein ABM2 comprises the VH and/or VL sequences of the binding domain designated as CD3-12.

618. The TBM of embodiment 593, wherein ABM2 comprises the heavy and light chain CDRs (as defined by Kabat, Chothia or a combination thereof) of the binding domain designated as CD3-13.

619. The TBM of embodiment 593, wherein ABM2 comprises the VH and/or VL sequences of the binding domain designated as CD3-13.

620. The TBM of embodiment 593, wherein ABM2 comprises the heavy and light chain CDRs (as defined by Kabat, Chothia or a combination thereof) of the binding domain designated as CD3-14.

621. The TBM of embodiment 593, wherein ABM2 comprises the VH and/or VL sequences of the binding domain designated as CD3-14.

622. The TBM of embodiment 593, wherein ABM2 comprises the heavy and light chain CDRs (as defined by Kabat, Chothia or a combination thereof) of the binding domain designated as CD3-15.

623. The TBM of embodiment 593, wherein ABM2 comprises the VH and/or VL sequences of the binding domain designated as CD3-15.

624. The TBM of embodiment 593, wherein ABM2 comprises the heavy and light chain CDRs (as defined by Kabat, Chothia or a combination thereof) of the binding domain designated as CD3-16.

625. The TBM of embodiment 593, wherein ABM2 comprises the VH and/or VL sequences of the binding domain designated as CD3-16.

626. The TBM of embodiment 593, wherein ABM2 comprises the heavy and light chain CDRs (as defined by Kabat, Chothia or a combination thereof) of the binding domain designated as CD3-17.

627. The TBM of embodiment 593, wherein ABM2 comprises the VH and/or VL sequences of the binding domain designated as CD3-17.

628. The TBM of embodiment 593, wherein ABM2 comprises the heavy and light chain CDRs (as defined by Kabat, Chothia or a combination thereof) of the binding domain designated as CD3-18.

629. The TBM of embodiment 593, wherein ABM2 comprises the VH and/or VL sequences of the binding domain designated as CD3-18.

630. The TBM of embodiment 593, wherein ABM2 comprises the heavy and light chain CDRs (as defined by Kabat, Chothia or a combination thereof) of the binding domain designated as CD3-19.

631. The TBM of embodiment 593, wherein ABM2 comprises the VH and/or VL sequences of the binding domain designated as CD3-19.

632. The TBM of embodiment 593, wherein ABM2 comprises the heavy and light chain CDRs (as defined by Kabat, Chothia or a combination thereof) of the binding domain designated as CD3-20.

633. The TBM of embodiment 593, wherein ABM2 comprises the VH and/or VL sequences of the binding domain designated as CD3-20.

634. The TBM of embodiment 593, wherein ABM2 comprises the heavy and light chain CDRs (as defined by Kabat, Chothia or a combination thereof) of the binding domain designated as CD3-21.

635. The TBM of embodiment 593, wherein ABM2 comprises the VH and/or VL sequences of the binding domain designated as CD3-21.

636. The TBM of embodiment 593, wherein ABM2 comprises the heavy and light chain CDRs (as defined by Kabat, Chothia or a combination thereof) of the binding domain designated as CD3-22.

637. The TBM of embodiment 593, wherein ABM2 comprises the VH and/or VL sequences of the binding domain designated as CD3-22.

638. The TBM of embodiment 593, wherein ABM2 comprises the heavy and light chain CDRs (as defined by Kabat, Chothia or a combination thereof) of the binding domain designated as CD3-23.

639. The TBM of embodiment 593, wherein ABM2 comprises the VH and/or VL sequences of the binding domain designated as CD3-23.

640. The TBM of embodiment 593, wherein ABM2 comprises the heavy and light chain CDRs (as defined by Kabat, Chothia or a combination thereof) of the binding domain designated as CD3-24.

641. The TBM of embodiment 593, wherein ABM2 comprises the VH and/or VL sequences of the binding domain designated as CD3-24.

642. The TBM of embodiment 593, wherein ABM2 comprises the heavy and light chain CDRs (as defined by Kabat, Chothia or a combination thereof) of the binding domain designated as CD3-25.

643. The TBM of embodiment 593, wherein ABM2 comprises the VH and/or VL sequences of the binding domain designated as CD3-25.

644. The TBM of embodiment 593, wherein ABM2 comprises the heavy and light chain CDRs (as defined by Kabat, Chothia or a combination thereof) of the binding domain designated as CD3-26.

645. The TBM of embodiment 593, wherein ABM2 comprises the VH and/or VL sequences of the binding domain designated as CD3-26.

646. The TBM of embodiment 593, wherein ABM2 comprises the heavy and light chain CDRs (as defined by Kabat, Chothia or a combination thereof) of the binding domain designated as CD3-27.

647. The TBM of embodiment 593, wherein ABM2 comprises the VH and/or VL sequences of the binding domain designated as CD3-27.

648. The TBM of embodiment 593, wherein ABM2 comprises the heavy and light chain CDRs (as defined by Kabat, Chothia or a combination thereof) of the binding domain designated as CD3-28.

649. The TBM of embodiment 593, wherein ABM2 comprises the VH and/or VL sequences of the binding domain designated as CD3-28.

650. The TBM of embodiment 592, wherein ABM2 comprises the heavy and light chain CDRs (as defined by Kabat) of any one of the binding domains designated as CD3-29 through CD3-128.

651. The TBM of embodiment 650, wherein ABM2 comprises the heavy and light chain CDRs (as defined by Kabat) of any one of the binding domains designated as CD3-29 through CD3-38.

652. The TBM of embodiment 650, wherein ABM2 comprises the heavy and light chain CDRs (as defined by Kabat) of any one of the binding domains designated as CD3-39 through CD3-48.

653. The TBM of embodiment 650, wherein ABM2 comprises the heavy and light chain CDRs (as defined by Kabat) of any one of the binding domains designated as CD3-49 through CD3-58.

654. The TBM of embodiment 650, wherein ABM2 comprises the heavy and light chain CDRs (as defined by Kabat) of any one of the binding domains designated as CD3-59 through CD3-68.

655. The TBM of embodiment 650, wherein ABM2 comprises the heavy and light chain CDRs (as defined by Kabat) of any one of the binding domains designated as CD3-69 through CD3-78.

656. The TBM of embodiment 650, wherein ABM2 comprises the heavy and light chain CDRs (as defined by Kabat) of any one of the binding domains designated as CD3-79 through CD3-88.

657. The TBM of embodiment 650, wherein ABM2 comprises the heavy and light chain CDRs (as defined by Kabat) of any one of the binding domains designated as CD3-89 through CD3-98.

658. The TBM of embodiment 650, wherein ABM2 comprises the heavy and light chain CDRs (as defined by Kabat) of any one of the binding domains designated as CD3-99 through CD3-108.

659. The TBM of embodiment 650, wherein ABM2 comprises the heavy and light chain CDRs (as defined by Kabat) of any one of the binding domains designated as CD3-109 through CD3-118.

660. The TBM of embodiment 650, wherein ABM2 comprises the heavy and light chain CDRs (as defined by Kabat) of any one of the binding domains designated as CD3-119 through CD3-128.

661. The TBM of any one of embodiments 1 to 589, wherein the component of a human TCR complex is the alpha subunit of the TCR.

662. The TBM of embodiment 661, wherein ABM2:
(a) an immunoglobulin scaffold-based ABM which is optionally an antibody, an antibody fragment, an scFv, a dsFv, a Fv, a Fab, an scFab, a (Fab′)2, a single domain antibody (SDAB), a VH or VL domain, or a camelid VHH domain; or
(b) a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.

663. The TBM of embodiment 662, wherein ABM2 comprises CDRs corresponding to the heavy and light chain CDRs of the antibody BMA031.

664. The TBM of embodiment 662, wherein ABM2 comprises variable regions corresponding to the VH and VL of the antibody BMA031.

665. The TBM of any one of embodiments 591 to 664, wherein ABM2 is an scFv.

666. The TBM of any one of embodiments 591 to 664, wherein ABM2 is a Fab.

667. The TBM of embodiment 666, wherein the Fab is a Fab heterodimer.

668. The TBM of any one of embodiments 1 to 667, wherein the TAA is a lineage marker.

669. The TBM of any one of embodiments 1 to 667, wherein the TAA is present on normal cells.

670. The TBM of embodiment 669, wherein the TAA is upregulated on cancer cells.

671. The TBM of any one of embodiments 1 to 670, wherein ABM3 is:
(a) an immunoglobulin scaffold-based ABM which is optionally an anti-TAA antibody, an antibody fragment, an scFv, a dsFv, a Fv, a Fab, an scFab, a (Fab′)2, a single domain antibody (SDAB), a VH or VL domain, or a camelid VHH domain; or
(b) a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.

672. The TBM of embodiment 668, wherein the TAA is TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, FLT3, TAAG72, CD22, CD33, GD2, BCMA, gp100Tn, FAP, tyrosinase, EPCAM, CEA, Igf-I receptor, or EphB2.

673. The TBM of embodiment 668, wherein ABM3 comprises the CDRs or variable region sequences of the antibodies set forth in Table 11.

674. The TBM of any one of embodiments 668 to 673, wherein the TAA is BCMA.

675. The TBM of embodiment 674, wherein ABM3 comprises any of the binding sequences set forth in any one of Tables 12A, 12B, 12C, 12D, 12E, 12F or 12G.

676. The TBM of embodiment 675, wherein ABM3 comprises the heavy and light chain CDRs (as defined by Kabat, Chothia or a combination thereof) of the antibody designated BCMA-1.

677. The TBM of embodiment 675, wherein ABM3 comprises the VH and/or VL sequences of the antibody designated as BCMA-1.

678. The TBM of embodiment 675, wherein ABM3 comprises the heavy and light chain CDRs (as defined by Kabat, Chothia or a combination thereof) of the antibody designated BCMA-2.

679. The TBM of embodiment 675, wherein ABM3 comprises the VH and/or VL sequences of the antibody designated as BCMA-2.

680. The TBM of embodiment 675, wherein ABM3 comprises the heavy and light chain CDRs (as defined by Kabat, Chothia or a combination thereof) of the antibody designated BCMA-3.

681. The TBM of embodiment 675, wherein ABM3 comprises the VH and/or VL sequences of the antibody designated as BCMA-3.

682. The TBM of embodiment 675, wherein ABM3 comprises the heavy and light chain CDRs (as defined by Kabat, Chothia or a combination thereof) of the antibody designated BCMA-4.

683. The TBM of embodiment 675, wherein ABM3 comprises the VH and/or VL sequences of the antibody designated as BCMA-4.

684. The TBM of embodiment 675, wherein ABM3 comprises the heavy and light chain CDRs (as defined by Kabat, Chothia or a combination thereof) of the antibody designated BCMA-5.

685. The TBM of embodiment 675, wherein ABM3 comprises the VH and/or VL sequences of the antibody designated as BCMA-5.

686. The TBM of embodiment 675, wherein ABM3 comprises the heavy and light chain CDRs (as defined by Kabat, Chothia or a combination thereof) of the antibody designated BCMA-6.

687. The TBM of embodiment 675, wherein ABM3 comprises the VH and/or VL sequences of the antibody designated as BCMA-6.

688. The TBM of embodiment 675, wherein ABM3 comprises the heavy and light chain CDRs (as defined by Kabat, Chothia or a combination thereof) of the antibody designated BCMA-7.

689. The TBM of embodiment 675, wherein ABM3 comprises the VH and/or VL sequences of the antibody designated as BCMA-7.

690. The TBM of embodiment 675, wherein ABM3 comprises the heavy and light chain CDRs (as defined by Kabat, Chothia or a combination thereof) of the antibody designated BCMA-8.

691. The TBM of embodiment 675, wherein ABM3 comprises the VH and/or VL sequences of the antibody designated as BCMA-8.

692. The TBM of embodiment 675, wherein ABM3 comprises the heavy and light chain CDRs (as defined by Kabat, Chothia or a combination thereof) of the antibody designated BCMA-9.

693. The TBM of embodiment 675, wherein ABM3 comprises the VH and/or VL sequences of the antibody designated as BCMA-9.

694. The TBM of embodiment 675, wherein ABM3 comprises the heavy and light chain CDRs (as defined by Kabat, Chothia or a combination thereof) of the antibody designated BCMA-10.

695. The TBM of embodiment 675, wherein ABM3 comprises the VH and/or VL sequences of the antibody designated as BCMA-10.

696. The TBM of embodiment 675, wherein ABM3 comprises the heavy and light chain CDRs (as defined by Kabat, Chothia or a combination thereof) of the antibody designated BCMA-11.

697. The TBM of embodiment 675, wherein ABM3 comprises the VH and/or VL sequences of the antibody designated as BCMA-11.

698. The TBM of embodiment 675, wherein ABM3 comprises the heavy and light chain CDRs (as defined by Kabat, Chothia or a combination thereof) of the antibody designated BCMA-12.

699. The TBM of embodiment 675, wherein ABM3 comprises the VH and/or VL sequences of the antibody designated as BCMA-12.

700. The TBM of embodiment 675, wherein ABM3 comprises the heavy and light chain CDRs (as defined by Kabat, Chothia or a combination thereof) of the antibody designated BCMA-13.

701. The TBM of embodiment 675, wherein ABM3 comprises the VH and/or VL sequences of the antibody designated as BCMA-13.

702. The TBM of embodiment 675, wherein ABM3 comprises the heavy and light chain CDRs (as defined by Kabat, Chothia or a combination thereof) of the antibody designated BCMA-14.

703. The TBM of embodiment 675, wherein ABM3 comprises the VH and/or VL sequences of the antibody designated as BCMA-14.

704. The TBM of embodiment 675, wherein ABM3 comprises the heavy and light chain CDRs (as defined by Kabat, Chothia or a combination thereof) of the antibody designated BCMA-15.

705. The TBM of embodiment 675, wherein ABM3 comprises the VH and/or VL sequences of the antibody designated as BCMA-15.

706. The TBM of embodiment 675, wherein ABM3 comprises the heavy and light chain CDRs (as defined by Kabat, Chothia or a combination thereof) of the antibody designated BCMA-16.

707. The TBM of embodiment 675, wherein ABM3 comprises the VH and/or VL sequences of the antibody designated as BCMA-16.

708. The TBM of embodiment 675, wherein ABM3 comprises the heavy and light chain CDRs (as defined by Kabat, Chothia or a combination thereof) of the antibody designated BCMA-17.

709. The TBM of embodiment 675, wherein ABM3 comprises the VH and/or VL sequences of the antibody designated as BCMA-17.

710. The TBM of embodiment 675, wherein ABM3 comprises the heavy and light chain CDRs (as defined by Kabat, Chothia or a combination thereof) of the antibody designated BCMA-18.

711. The TBM of embodiment 675, wherein ABM3 comprises the VH and/or VL sequences of the antibody designated as BCMA-18.

712. The TBM of embodiment 675, wherein ABM3 comprises the heavy and light chain CDRs (as defined by Kabat, Chothia or a combination thereof) of the antibody designated BCMA-19.

713. The TBM of embodiment 675, wherein ABM3 comprises the VH and/or VL sequences of the antibody designated as BCMA-19.

714. The TBM of embodiment 675, wherein ABM3 comprises the heavy and light chain CDRs (as defined by Kabat, Chothia or a combination thereof) of the antibody designated BCMA-20.

715. The TBM of embodiment 675, wherein ABM3 comprises the VH and/or VL sequences of the antibody designated as BCMA-20.

716. The TBM of embodiment 675, wherein ABM3 comprises the heavy and light chain CDRs (as defined by Kabat, Chothia or a combination thereof) of the antibody designated BCMA-21.

717. The TBM of embodiment 675, wherein ABM3 comprises the VH and/or VL sequences of the antibody designated as BCMA-21.

718. The TBM of embodiment 675, wherein ABM3 comprises the heavy and light chain CDRs (as defined by Kabat, Chothia or a combination thereof) of the antibody designated BCMA-22.

719. The TBM of embodiment 675, wherein ABM3 comprises the VH and/or VL sequences of the antibody designated as BCMA-22.

720. The TBM of embodiment 675, wherein ABM3 comprises the heavy and light chain CDRs (as defined by Kabat, Chothia or a combination thereof) of the antibody designated BCMA-23.

721. The TBM of embodiment 675, wherein ABM3 comprises the VH and/or VL sequences of the antibody designated as BCMA-23.

722. The TBM of embodiment 675, wherein ABM3 comprises the heavy and light chain CDRs (as defined by Kabat, Chothia or a combination thereof) of the antibody designated BCMA-24.

723. The TBM of embodiment 675, wherein ABM3 comprises the VH and/or VL sequences of the antibody designated as BCMA-24.

724. The TBM of embodiment 675, wherein ABM3 comprises the heavy and light chain CDRs (as defined by Kabat, Chothia or a combination thereof) of the antibody designated BCMA-25.

725. The TBM of embodiment 675, wherein ABM3 comprises the VH and/or VL sequences of the antibody designated as BCMA-25.

726. The TBM of embodiment 675, wherein ABM3 comprises the heavy and light chain CDRs (as defined by Kabat, Chothia or a combination thereof) of the antibody designated BCMA-26.

727. The TBM of embodiment 675, wherein ABM3 comprises the VH and/or VL sequences of the antibody designated as BCMA-26.

728. The TBM of embodiment 675, wherein ABM3 comprises the heavy and light chain CDRs (as defined by Kabat, Chothia or a combination thereof) of the antibody designated BCMA-27.

729. The TBM of embodiment 675, wherein ABM3 comprises the VH and/or VL sequences of the antibody designated as BCMA-27.

730. The TBM of embodiment 675, wherein ABM3 comprises the heavy and light chain CDRs (as defined by Kabat, Chothia or a combination thereof) of the antibody designated BCMA-28.

731. The TBM of embodiment 675, wherein ABM3 comprises the VH and/or VL sequences of the antibody designated as BCMA-28.

732. The TBM of embodiment 675, wherein ABM3 comprises the heavy and light chain CDRs (as defined by Kabat, Chothia or a combination thereof) of the antibody designated BCMA-29.

733. The TBM of embodiment 675, wherein ABM3 comprises the VH and/or VL sequences of the antibody designated as BCMA-29.

734. The TBM of embodiment 675, wherein ABM3 comprises the heavy and light chain CDRs (as defined by Kabat, Chothia or a combination thereof) of the antibody designated BCMA-30.

735. The TBM of embodiment 675, wherein ABM3 comprises the VH and/or VL sequences of the antibody designated as BCMA-30.

736. The TBM of embodiment 675, wherein ABM3 comprises the heavy and light chain CDRs (as defined by Kabat, Chothia or a combination thereof) of the antibody designated BCMA-31.

737. The TBM of embodiment 675, wherein ABM3 comprises the VH and/or VL sequences of the antibody designated as BCMA-31.

738. The TBM of embodiment 675, wherein ABM3 comprises the heavy and light chain CDRs (as defined by Kabat, Chothia or a combination thereof) of the antibody designated BCMA-32.

739. The TBM of embodiment 675, wherein ABM3 comprises the VH and/or VL sequences of the antibody designated as BCMA-32.

740. The TBM of embodiment 675, wherein ABM3 comprises the heavy and light chain CDRs (as defined by Kabat, Chothia or a combination thereof) of the antibody designated BCMA-33.

741. The TBM of embodiment 675, wherein ABM3 comprises the VH and/or VL sequences of the antibody designated as BCMA-33.

742. The TBM of embodiment 675, wherein ABM3 comprises the heavy and light chain CDRs (as defined by Kabat, Chothia or a combination thereof) of the antibody designated BCMA-34.

743. The TBM of embodiment 675, wherein ABM3 comprises the VH and/or VL sequences of the antibody designated as BCMA-34.

744. The TBM of embodiment 675, wherein ABM3 comprises the heavy and light chain CDRs (as defined by Kabat, Chothia or a combination thereof) of the antibody designated BCMA-35.

745. The TBM of embodiment 675, wherein ABM3 comprises the VH and/or VL sequences of the antibody designated as BCMA-35.

746. The TBM of embodiment 675, wherein ABM3 comprises the heavy and light chain CDRs (as defined by Kabat, Chothia or a combination thereof) of the antibody designated BCMA-36.

747. The TBM of embodiment 675, wherein ABM3 comprises the VH and/or VL sequences of the antibody designated as BCMA-36.

748. The TBM of embodiment 675, wherein ABM3 comprises the heavy and light chain CDRs (as defined by Kabat, Chothia or a combination thereof) of the antibody designated BCMA-37.

749. The TBM of embodiment 675, wherein ABM3 comprises the VH and/or VL sequences of the antibody designated as BCMA-37.

750. The TBM of embodiment 675, wherein ABM3 comprises the heavy and light chain CDRs (as defined by Kabat, Chothia or a combination thereof) of the antibody designated BCMA-38.

751. The TBM of embodiment 675, wherein ABM3 comprises the VH and/or VL sequences of the antibody designated as BCMA-38.

752. The TBM of embodiment 675, wherein ABM3 comprises the heavy and light chain CDRs (as defined by Kabat, Chothia or a combination thereof) of the antibody designated BCMA-39.

753. The TBM of embodiment 675, wherein ABM3 comprises the VH and/or VL sequences of the antibody designated as BCMA-39.

754. The TBM of embodiment 675, wherein ABM3 comprises the heavy and light chain CDRs (as defined by Kabat, Chothia or a combination thereof) of the antibody designated BCMA-40.

755. The TBM of embodiment 675, wherein ABM3 comprises the VH and/or VL sequences of the antibody designated as BCMA-40.

756. The TBM of any one of embodiments 668 to 673, wherein the TAA is CD19.

757. The TBM of embodiment 756, wherein ABM3 comprises any of the binding sequences set forth Table 13.

758. The TBM of embodiment 757, wherein ABM3 comprises:
(a) a CDR-H1 having the amino acid sequence of the CDR designated as CD19-H1;
(b) a CDR-H2 having the amino acid sequence of any one of the CDRs designated as CD19-H2A, HD19-H2B, CD19-H2C and CD19-H2D;
(c) a CDR-H3 having the amino acid sequence of the CDR designated as CD19-H3;
(d) a CDR-L1 having the amino acid sequence of the CDR designated as CD19-L1;
(e) a CDR-L2 having the amino acid sequence of the CDR designated as CD19-L2; and
(f) a CDR-L3 having the amino acid sequence of the CDR designated as CD19-L23.

759. The TBM of embodiment 757, wherein ABM3 comprises:
(a) a VH having the amino acid sequence of any one of the VH's designated as CD19-VHA, CD19-VHB, CD19-VHC, and CD19-VHD; and
(b) a VL having the amino acid sequence of any one of the VL's designated as CD19-VLA and CD19-VLB.

760. The TBM of any one of embodiments any one of embodiments 668 to 759, wherein ABM3 is an scFv.

761. The TBM of any one of embodiments 668 to 757, wherein ABM3 is a Fab.

762. The TBM of embodiment 761, wherein the Fab is a Fab heterodimer.

763. The TBM of any one of embodiments 1 to 762 which comprises an Fc domain.

764. The TBM of embodiment 763, wherein the Fc domain is an Fc heterodimer.

765. The TBM of embodiment 764, wherein the Fc heterodimer comprises knob-in-hole (“KIH”) modifications.

766. The TBM of embodiment 765, wherein the KIH modifications are any of the KIH modifications described in Section 4.3.1.5.1 or in Table 2.

767. The TBM of embodiment 765, wherein the KIH modifications are any of the alternative KIH modifications described in Section 4.3.1.5.2 or in Table 2.

768. The TBM of any one of embodiments 764 to 767, which comprises polar bridge modifications.

769. The TBM of embodiment 768, wherein the polar bridge modification are any of the polar bridge modifications described in Section 4.3.1.5.3 or in Table 2.

770. The TBM of any one of embodiments to 764 to 769, which comprises at least one of the Fc modifications designated as Fc 1 through Fc 150.

771. The TBM of embodiment 770, which comprises at least one of the Fc modifications designated as Fc 1 through Fc 5.

772. The TBM of embodiment 770, which comprises at least one of the Fc modifications designated as Fc 6 through Fc 10.

773. The TBM of embodiment 770, which comprises at least one of the Fc modifications designated as Fc 11 through Fc 15.

774. The TBM of embodiment 770, which comprises at least one of the Fc modifications designated as Fc 16 through Fc 20.

775. The TBM of embodiment 770, which comprises at least one of the Fc modifications designated as Fc 21 through Fc 25.

776. The TBM of embodiment 770, which comprises at least one of the Fc modifications designated as Fc 26 through Fc 30.

777. The TBM of embodiment 770, which comprises at least one of the Fc modifications designated as Fc 31 through Fc 35.

778. The TBM of embodiment 770, which comprises at least one of the Fc modifications designated as Fc 36 through Fc 40.

779. The TBM of embodiment 770, which comprises at least one of the Fc modifications designated as Fc 41 through Fc 45.

780. The TBM of embodiment 770, which comprises at least one of the Fc modifications designated as Fc 46 through Fc 50.

781. The TBM of embodiment 770, which comprises at least one of the Fc modifications designated as Fc 51 through Fc 55.

782. The TBM of embodiment 770, which comprises at least one of the Fc modifications designated as Fc 56 through Fc 60.

783. The TBM of embodiment 770, which comprises at least one of the Fc modifications designated as Fc 61 through Fc 65.

784. The TBM of embodiment 770, which comprises at least one of the Fc modifications designated as Fc 66 through Fc 70.

785. The TBM of embodiment 770, which comprises at least one of the Fc modifications designated as Fc 71 through Fc 75.

786. The TBM of embodiment 770, which comprises at least one of the Fc modifications designated as Fc 76 through Fc 80.

787. The TBM of embodiment 770, which comprises at least one of the Fc modifications designated as Fc 81 through Fc 85.

788. The TBM of embodiment 770, which comprises at least one of the Fc modifications designated as Fc 86 through Fc 90.

789. The TBM of embodiment 770, which comprises at least one of the Fc modifications designated as Fc 91 through Fc 95.

790. The TBM of embodiment 770, which comprises at least one of the Fc modifications designated as Fc 96 through Fc 100.

791. The TBM of embodiment 770, which comprises at least one of the Fc modifications designated as Fc 101 through Fc 105.

792. The TBM of embodiment 770, which comprises at least one of the Fc modifications designated as Fc 106 through Fc 110.

793. The TBM of embodiment 770, which comprises at least one of the Fc modifications designated as Fc 111 through Fc 115.

794. The TBM of embodiment 770, which comprises at least one of the Fc modifications designated as Fc 116 through Fc 120.

795. The TBM of embodiment 770, which comprises at least one of the Fc modifications designated as Fc 121 through Fc 125.

796. The TBM of embodiment 770, which comprises at least one of the Fc modifications designated as Fc 126 through Fc 130.

797. The TBM of embodiment 770, which comprises at least one of the Fc modifications designated as Fc 131 through Fc 135.

798. The TBM of embodiment 770, which comprises at least one of the Fc modifications designated as Fc 136 through Fc 140.

799. The TBM of embodiment 770, which comprises at least one of the Fc modifications designated as Fc 141 through Fc 145.

800. The TBM of embodiment 770, which comprises at least one of the Fc modifications designated as Fc 146 through Fc 150.

801. The TBM of any one of embodiments 763 to 800, wherein the Fc domain has altered effector function.

802. The TBM of embodiment 801, wherein the Fc domain has altered binding to one or more Fc receptors.

803. The TBM of embodiment 802, wherein the one or more Fc receptors comprise FcRN.

804. The TBM of embodiment 802 or embodiment 803, wherein the one or more Fc receptors comprise leukocyte receptors.

805. The TBM of any one of embodiments 763 to 804, wherein the Fc has modified disulfide bond architecture.

806. The TBM of any one of embodiments 763 to 805, wherein the Fc has altered glycosylation patterns.

807. The TBM of any one of embodiments 763 to 806, wherein the Fc comprises a hinge region.

808. The TBM of embodiment 807, wherein the hinge region comprises any of the hinge regions described in Section 4.3.2.

809. The TBM of embodiment 808, wherein the hinge region comprises the amino acid sequence of the hinge region designated H1.

810. The TBM of embodiment 808, wherein the hinge region comprises the amino acid sequence of the hinge region designated H2.

811. The TBM of embodiment 808, wherein the hinge region comprises the amino acid sequence of the hinge region designated H3.

812. The TBM of embodiment 808, wherein the hinge region comprises the amino acid sequence of the hinge region designated H4.

813. The TBM of embodiment 808, wherein the hinge region comprises the amino acid sequence of the hinge region designated H5.

814. The TBM of embodiment 808, wherein the hinge region comprises the amino acid sequence of the hinge region designated H6.

815. The TBM of embodiment 808, wherein the hinge region comprises the amino acid sequence of the hinge region designated H7.

816. The TBM of embodiment 808, wherein the hinge region comprises the amino acid sequence of the hinge region designated H8.

817. The TBM of embodiment 808, wherein the hinge region comprises the amino acid sequence of the hinge region designated H9.

818. The TBM of embodiment 808, wherein the hinge region comprises the amino acid sequence of the hinge region designated H10.

819. The TBM of embodiment 808, wherein the hinge region comprises the amino acid sequence of the hinge region designated H11.

820. The TBM of embodiment 808, wherein the hinge region comprises the amino acid sequence of the hinge region designated H12.

821. The TBM of embodiment 808, wherein the hinge region comprises the amino acid sequence of the hinge region designated H13.

822. The TBM of embodiment 808, wherein the hinge region comprises the amino acid sequence of the hinge region designated H14.

823. The TBM of embodiment 808, wherein the hinge region comprises the amino acid sequence of the hinge region designated H15.

824. The TBM of embodiment 808, wherein the hinge region comprises the amino acid sequence of the hinge region designated H16.

825. The TBM of embodiment 808, wherein the hinge region comprises the amino acid sequence of the hinge region designated H17.

826. The TBM of embodiment 808, wherein the hinge region comprises the amino acid sequence of the hinge region designated H18.

827. The TBM of embodiment 808, wherein the hinge region comprises the amino acid sequence of the hinge region designated H19.

828. The TBM of embodiment 808, wherein the hinge region comprises the amino acid sequence of the hinge region designated H20.

829. The TBM of embodiment 808, wherein the hinge region comprises the amino acid sequence of the hinge region designated H21.

830. The TBM of any one of embodiments 1 to 829, which comprises at least one scFv domain.

831. The TBM of embodiment 830, wherein at least one scFv comprises a linker connecting the VH and VL domains.

832. The TBM of embodiment 831, wherein the linker is 5 to 25 amino acids in length.

833. The TBM of embodiment 832, wherein the linker is 12 to 20 amino acids in length.

834. The TBM of any one of embodiments 831 to 833, wherein the linker is a charged linker and/or a flexible linker.

835. The TBM of any one of embodiments 831 to 834, wherein the linker is selected from any one of linkers L1 through L54.

836. The TBM of embodiment 835, wherein the linker region comprises the amino acid sequence of the linker designated L1.

837. The TBM of embodiment 835, wherein the linker region comprises the amino acid sequence of the linker designated L2.

838. The TBM of embodiment 835, wherein the linker region comprises the amino acid sequence of the linker designated L3.

839. The TBM of embodiment 835, wherein the linker region comprises the amino acid sequence of the linker designated L4.

840. The TBM of embodiment 835, wherein the linker region comprises the amino acid sequence of the linker designated L5.

841. The TBM of embodiment 835, wherein the linker region comprises the amino acid sequence of the linker designated L6.

842. The TBM of embodiment 835, wherein the linker region comprises the amino acid sequence of the linker designated L7.

843. The TBM of embodiment 835, wherein the linker region comprises the amino acid sequence of the linker designated L8.

844. The TBM of embodiment 835, wherein the linker region comprises the amino acid sequence of the linker designated L9.

845. The TBM of embodiment 835, wherein the linker region comprises the amino acid sequence of the linker designated L10.

846. The TBM of embodiment 835, wherein the linker region comprises the amino acid sequence of the linker designated L11.

847. The TBM of embodiment 835, wherein the linker region comprises the amino acid sequence of the linker designated L12.

848. The TBM of embodiment 835, wherein the linker region comprises the amino acid sequence of the linker designated L13.

849. The TBM of embodiment 835, wherein the linker region comprises the amino acid sequence of the linker designated L14.

850. The TBM of embodiment 835, wherein the linker region comprises the amino acid sequence of the linker designated L15.

851. The TBM of embodiment 835, wherein the linker region comprises the amino acid sequence of the linker designated L16.

852. The TBM of embodiment 835, wherein the linker region comprises the amino acid sequence of the linker designated L17.

853. The TBM of embodiment 835, wherein the linker region comprises the amino acid sequence of the linker designated L18.

854. The TBM of embodiment 835, wherein the linker region comprises the amino acid sequence of the linker designated L19.

855. The TBM of embodiment 835, wherein the linker region comprises the amino acid sequence of the linker designated L20.

856. The TBM of embodiment 835, wherein the linker region comprises the amino acid sequence of the linker designated L21.

857. The TBM of embodiment 835, wherein the linker region comprises the amino acid sequence of the linker designated L22.

858. The TBM of embodiment 835, wherein the linker region comprises the amino acid sequence of the linker designated L23.

859. The TBM of embodiment 835, wherein the linker region comprises the amino acid sequence of the linker designated L24.

860. The TBM of embodiment 835, wherein the linker region comprises the amino acid sequence of the linker designated L25.

861. The TBM of embodiment 835, wherein the linker region comprises the amino acid sequence of the linker designated L26.

862. The TBM of embodiment 835, wherein the linker region comprises the amino acid sequence of the linker designated L27.

863. The TBM of embodiment 835, wherein the linker region comprises the amino acid sequence of the linker designated L28.

864. The TBM of embodiment 835, wherein the linker region comprises the amino acid sequence of the linker designated L29.

865. The TBM of embodiment 835, wherein the linker region comprises the amino acid sequence of the linker designated L30.

866. The TBM of embodiment 835, wherein the linker region comprises the amino acid sequence of the linker designated L31.

867. The TBM of embodiment 835, wherein the linker region comprises the amino acid sequence of the linker designated L32.

868. The TBM of embodiment 835, wherein the linker region comprises the amino acid sequence of the linker designated L33.

869. The TBM of embodiment 835, wherein the linker region comprises the amino acid sequence of the linker designated L34.

870. The TBM of embodiment 835, wherein the linker region comprises the amino acid sequence of the linker designated L35.

871. The TBM of embodiment 835, wherein the linker region comprises the amino acid sequence of the linker designated L36.

872. The TBM of embodiment 835, wherein the linker region comprises the amino acid sequence of the linker designated L37.

873. The TBM of embodiment 835, wherein the linker region comprises the amino acid sequence of the linker designated L38.

874. The TBM of embodiment 835, wherein the linker region comprises the amino acid sequence of the linker designated L39.

875. The TBM of embodiment 835, wherein the linker region comprises the amino acid sequence of the linker designated L40.

876. The TBM of embodiment 835, wherein the linker region comprises the amino acid sequence of the linker designated L41.

877. The TBM of embodiment 835, wherein the linker region comprises the amino acid sequence of the linker designated L42.

878. The TBM of embodiment 835, wherein the linker region comprises the amino acid sequence of the linker designated L43.

879. The TBM of embodiment 835, wherein the linker region comprises the amino acid sequence of the linker designated L44.

880. The TBM of embodiment 835, wherein the linker region comprises the amino acid sequence of the linker designated L45.

881. The TBM of embodiment 835, wherein the linker region comprises the amino acid sequence of the linker designated L46.

882. The TBM of embodiment 835, wherein the linker region comprises the amino acid sequence of the linker designated L47.

883. The TBM of embodiment 835, wherein the linker region comprises the amino acid sequence of the linker designated L48.

884. The TBM of embodiment 835, wherein the linker region comprises the amino acid sequence of the linker designated L49.

885. The TBM of embodiment 835, wherein the linker region comprises the amino acid sequence of the linker designated L50.

886. The TBM of embodiment 835, wherein the linker region comprises the amino acid sequence of the linker designated L51.

887. The TBM of embodiment 835, wherein the linker region comprises the amino acid sequence of the linker designated L52.

888. The TBM of embodiment 835, wherein the linker region comprises the amino acid sequence of the linker designated L53.

889. The TBM of embodiment 835, wherein the linker region comprises the amino acid sequence of the linker designated L54.

890. The TBM of any one of embodiments 1 to 889, which comprises at least one Fab domain.

891. The TBM of embodiment 890, wherein at least one Fab domain comprises any of the Fab heterodimerization modifications set forth in Table 1.

892. The TBM of embodiment 891, wherein at least one Fab domain comprises the Fab heterodimerization modifications designated as F1.

893. The TBM of embodiment 891, wherein at least one Fab domain comprises the Fab heterodimerization modifications designated as F2.

894. The TBM of embodiment 891, wherein at least one Fab domain comprises the Fab heterodimerization modifications designated as F3.

895. The TBM of embodiment 891, wherein at least one Fab domain comprises the Fab heterodimerization modifications designated as F4.

896. The TBM of embodiment 891, wherein at least one Fab domain comprises the Fab heterodimerization modifications designated as F5.

897. The TBM of embodiment 891, wherein at least one Fab domain comprises the Fab heterodimerization modifications designated as F6.

898. The TBM of embodiment 891, wherein at least one Fab domain comprises the Fab heterodimerization modifications designated as F7.

899. The TBM of any one of embodiments 1 to 898, which comprises at least two ABMs, an ABM and an ABM chain, or two ABM chains connected to one another via a linker.

900. The TBM of embodiment 899, wherein the linker is 5 to 25 amino acids in length.

901. The TBM of embodiment 900, wherein the linker is 12 to 20 amino acids in length.

902. The TBM of any one of embodiments 899 to 901, wherein the linker is a charged linker and/or a flexible linker.

903. The TBM of any one of embodiments 899 to 902, wherein the linker is selected from any one of linkers L1 through L54.

904. The TBM of embodiment 903, wherein the linker region comprises the amino acid sequence of the linker designated L1.

905. The TBM of embodiment 903, wherein the linker region comprises the amino acid sequence of the linker designated L2.

906. The TBM of embodiment 903, wherein the linker region comprises the amino acid sequence of the linker designated L3.

907. The TBM of embodiment 903, wherein the linker region comprises the amino acid sequence of the linker designated L4.

908. The TBM of embodiment 903, wherein the linker region comprises the amino acid sequence of the linker designated L5.

909. The TBM of embodiment 903, wherein the linker region comprises the amino acid sequence of the linker designated L6.

910. The TBM of embodiment 903, wherein the linker region comprises the amino acid sequence of the linker designated L7.

911. The TBM of embodiment 903, wherein the linker region comprises the amino acid sequence of the linker designated L8.

912. The TBM of embodiment 903, wherein the linker region comprises the amino acid sequence of the linker designated L9.

913. The TBM of embodiment 903, wherein the linker region comprises the amino acid sequence of the linker designated L10.

914. The TBM of embodiment 903, wherein the linker region comprises the amino acid sequence of the linker designated L11.

915. The TBM of embodiment 903, wherein the linker region comprises the amino acid sequence of the linker designated L12.

916. The TBM of embodiment 903, wherein the linker region comprises the amino acid sequence of the linker designated L13.

917. The TBM of embodiment 903, wherein the linker region comprises the amino acid sequence of the linker designated L14.

918. The TBM of embodiment 903, wherein the linker region comprises the amino acid sequence of the linker designated L15.

919. The TBM of embodiment 903, wherein the linker region comprises the amino acid sequence of the linker designated L16.

920. The TBM of embodiment 903, wherein the linker region comprises the amino acid sequence of the linker designated L17.

921. The TBM of embodiment 903, wherein the linker region comprises the amino acid sequence of the linker designated L18.

922. The TBM of embodiment 903, wherein the linker region comprises the amino acid sequence of the linker designated L19.

923. The TBM of embodiment 903, wherein the linker region comprises the amino acid sequence of the linker designated L20.

924. The TBM of embodiment 903, wherein the linker region comprises the amino acid sequence of the linker designated L21.

925. The TBM of embodiment 903, wherein the linker region comprises the amino acid sequence of the linker designated L22.

926. The TBM of embodiment 903, wherein the linker region comprises the amino acid sequence of the linker designated L23.

927. The TBM of embodiment 903, wherein the linker region comprises the amino acid sequence of the linker designated L24.

928. The TBM of embodiment 903, wherein the linker region comprises the amino acid sequence of the linker designated L25.

929. The TBM of embodiment 903, wherein the linker region comprises the amino acid sequence of the linker designated L26.

930. The TBM of embodiment 903, wherein the linker region comprises the amino acid sequence of the linker designated L27.

931. The TBM of embodiment 903, wherein the linker region comprises the amino acid sequence of the linker designated L28.

932. The TBM of embodiment 903, wherein the linker region comprises the amino acid sequence of the linker designated L29.

933. The TBM of embodiment 903, wherein the linker region comprises the amino acid sequence of the linker designated L30.

934. The TBM of embodiment 903, wherein the linker region comprises the amino acid sequence of the linker designated L31.

935. The TBM of embodiment 903, wherein the linker region comprises the amino acid sequence of the linker designated L32.

936. The TBM of embodiment 903, wherein the linker region comprises the amino acid sequence of the linker designated L33.

937. The TBM of embodiment 903, wherein the linker region comprises the amino acid sequence of the linker designated L34.

938. The TBM of embodiment 903, wherein the linker region comprises the amino acid sequence of the linker designated L35.

939. The TBM of embodiment 903, wherein the linker region comprises the amino acid sequence of the linker designated L36.

940. The TBM of embodiment 903, wherein the linker region comprises the amino acid sequence of the linker designated L37.

941. The TBM of embodiment 903, wherein the linker region comprises the amino acid sequence of the linker designated L38.

942. The TBM of embodiment 903, wherein the linker region comprises the amino acid sequence of the linker designated L39.

943. The TBM of embodiment 903, wherein the linker region comprises the amino acid sequence of the linker designated L40.

944. The TBM of embodiment 903, wherein the linker region comprises the amino acid sequence of the linker designated L41.

945. The TBM of embodiment 903, wherein the linker region comprises the amino acid sequence of the linker designated L42.

946. The TBM of embodiment 903, wherein the linker region comprises the amino acid sequence of the linker designated L43.

947. The TBM of embodiment 903, wherein the linker region comprises the amino acid sequence of the linker designated L44.

948. The TBM of embodiment 903, wherein the linker region comprises the amino acid sequence of the linker designated L45.

949. The TBM of embodiment 903, wherein the linker region comprises the amino acid sequence of the linker designated L46.

950. The TBM of embodiment 903, wherein the linker region comprises the amino acid sequence of the linker designated L47.

951. The TBM of embodiment 903, wherein the linker region comprises the amino acid sequence of the linker designated L48.

952. The TBM of embodiment 903, wherein the linker region comprises the amino acid sequence of the linker designated L49.

953. The TBM of embodiment 835, wherein the linker region comprises the amino acid sequence of the linker designated L50.

954. The TBM of embodiment 835, wherein the linker region comprises the amino acid sequence of the linker designated L51.

955. The TBM of embodiment 835, wherein the linker region comprises the amino acid sequence of the linker designated L52.

956. The TBM of embodiment 835, wherein the linker region comprises the amino acid sequence of the linker designated L53.

957. The TBM of embodiment 903, wherein the linker region comprises the amino acid sequence of the linker designated L54.

958. The TBM of any one of embodiments 1 to 957, which is a trivalent TBM.

959. The TBM of embodiment 958, wherein the trivalent TBM has any one of the configurations depicted in FIGS. 1B-1O and 1U-1Z.

960. The TBM of embodiment 959, wherein the trivalent TBM has the configuration depicted in FIG. 1B.

961. The TBM of embodiment 959, wherein the trivalent TBM has the configuration depicted in FIG. 1C.

962. The TBM of embodiment 959, wherein the trivalent TBM has the configuration depicted in FIG. 1D.

963. The TBM of embodiment 959, wherein the trivalent TBM has the configuration depicted in FIG. 1E.

964. The TBM of embodiment 959, wherein the trivalent TBM has the configuration depicted in FIG. 1F.

965. The TBM of embodiment 959, wherein the trivalent TBM has the configuration depicted in FIG. 1G.

966. The TBM of embodiment 959, wherein the trivalent TBM has the configuration depicted in FIG. 1H.

967. The TBM of embodiment 959, wherein the trivalent TBM has the configuration depicted in FIG. 1I.

968. The TBM of embodiment 959, wherein the trivalent TBM has the configuration depicted in FIG. 1J.

969. The TBM of embodiment 959, wherein the trivalent TBM has the configuration depicted in FIG. 1K.

970. The TBM of embodiment 959, wherein the trivalent TBM has the configuration depicted in FIG. 1L.

971. The TBM of embodiment 959, wherein the trivalent TBM has the configuration depicted in FIG. 1M.

972. The TBM of embodiment 959, wherein the trivalent TBM has the configuration depicted in FIG. 1N.

973. The TBM of embodiment 959, wherein the trivalent TBM has the configuration depicted in FIG. 1O.

974. The TBM of embodiment 959, wherein the trivalent TBM has the configuration depicted in FIG. 1V.

975. The TBM of embodiment 959, wherein the trivalent TBM has the configuration depicted in FIG. 1W.

976. The TBM of embodiment 959, wherein the trivalent TBM has the configuration depicted in FIG. 1X.

977. The TBM of embodiment 959, wherein the trivalent TBM has the configuration depicted in FIG. 1Y.

978. The TBM of embodiment 959, wherein the trivalent TBM has the configuration depicted in FIG. 1Z.

979. The TBM of any one of embodiments 959 to 978, in which the ABMs have the configuration designated as T1.

980. The TBM of any one of embodiments 959 to 978, in which the ABMs have the configuration designated as T2.

981. The TBM of any one of embodiments 959 to 978, in which the ABMs have the configuration designated as T3.

982. The TBM of any one of embodiments 959 to 978, in which the ABMs have the configuration designated as T4.

983. The TBM of any one of embodiments 959 to 978, in which the ABMs have the configuration designated as T5.

984. The TBM of any one of embodiments 959 to 978, in which the ABMs have the configuration designated as T6.

985. The TBM of any one of embodiments 1 to 957, which is a tetravalent TBM.

986. The TBM of embodiment 985, wherein the tetravalent TBM has any one of the configurations depicted in FIGS. 1P-1R.

987. The TBM of embodiment 986, wherein the tetravalent TBM has the configuration depicted in FIG. 1P.

988. The TBM of embodiment 986, wherein the tetravalent TBM has the configuration depicted in FIG. 1Q.

989. The TBM of embodiment 986, wherein the tetravalent TBM has the configuration depicted in FIG. 1R.

990. The TBM of any one of embodiments 986 to 989, in which the ABMs have any of the configurations designated Tv 1 through Tv 12.

991. The TBM of embodiment 990, in which the ABMs have the configuration designated Tv 1.

992. The TBM of embodiment 990, in which the ABMs have the configuration designated Tv 2.

993. The TBM of embodiment 990, in which the ABMs have the configuration designated Tv 3.

994. The TBM of embodiment 990, in which the ABMs have the configuration designated Tv 4.

995. The TBM of embodiment 990, in which the ABMs have the configuration designated Tv 5.

996. The TBM of embodiment 990, in which the ABMs have the configuration designated Tv 6.

997. The TBM of embodiment 990, in which the ABMs have the configuration designated Tv 7.

998. The TBM of embodiment 990, in which the ABMs have the configuration designated Tv 8.

999. The TBM of embodiment 990, in which the ABMs have the configuration designated Tv 9.

1000. The TBM of embodiment 990, in which the ABMs have the configuration designated Tv 10.

1001. The TBM of embodiment 990, in which the ABMs have the configuration designated Tv 11.

1002. The TBM of embodiment 990, in which the ABMs have the configuration designated Tv 12.

1003. The TBM of any one of embodiments 1 to 957, which is a pentavalent TBM.

1004. The TBM of embodiment 1003, wherein the pentavalent TBM has the configuration depicted in FIG. 1s.

1005. The TBM of embodiment 1004, in which the ABMs have any of the configurations designated Pv 1 through Pv 80.

1006. The TBM of embodiment 1005, in which the ABMs have a configuration selected from any one of the configurations designated Pv 1 through Pv 10.

1007. The TBM of embodiment 1005, in which the ABMs have a configuration selected from any one of the configurations designated Pv 11 through Pv 20.

1008. The TBM of embodiment 1005, in which the ABMs have a configuration selected from any one of the configurations designated Pv 21 through Pv 30.

1009. The TBM of embodiment 1005, in which the ABMs have a configuration selected from any one of the configurations designated Pv 31 through Pv 40.

1010. The TBM of embodiment 1005, in which the ABMs have a configuration selected from any one of the configurations designated Pv 41 through Pv 50.

1011. The TBM of embodiment 1005, in which the ABMs have a configuration selected from any one of the configurations designated Pv 51 through Pv 60.

1012. The TBM of embodiment 1005, in which the ABMs have a configuration selected from any one of the configurations designated Pv 61 through Pv 70.

1013. The TBM of embodiment 1005, in which the ABMs have a configuration selected from any one of the configurations designated Pv 71 through Pv 80.

1014. The TBM of any one of embodiments 1 to 957, which is a hexavalent TBM.

1015. The TBM of embodiment 1014, wherein the hexavalent TBM has the configuration depicted in FIG. 1T or FIG. 1U.

1016. The TBM of embodiment 1015, wherein the hexavalent TBM has the configuration depicted in FIG. 1T.

1017. The TBM of embodiment 1015, wherein the hexavalent TBM has the configuration depicted in FIG. 1U.

1018. The TBM of any one of embodiments 1015 to 1017, in which the ABMs have any of the configurations designated Hv 1 through Hv 240.

1019. The TBM of embodiment 1018, in which the ABMs have a configuration selected from any one of the configurations designated Hv 1 through Hv 10.

1020. The TBM of embodiment 1018, in which the ABMs have a configuration selected from any one of the configurations designated Hv 11 through Hv 20.

1021. The TBM of embodiment 1018, in which the ABMs have a configuration selected from any one of the configurations designated Hv 21 through Hv 30.

1022. The TBM of embodiment 1018, in which the ABMs have a configuration selected from any one of the configurations designated Hv 31 through Hv 40.

1023. The TBM of embodiment 1018, in which the ABMs have a configuration selected from any one of the configurations designated Hv 41 through Hv 50.

1024. The TBM of embodiment 1018, in which the ABMs have a configuration selected from any one of the configurations designated Hv 51 through Hv 60.

1025. The TBM of embodiment 1018, in which the ABMs have a configuration selected from any one of the configurations designated Hv 61 through Hv 70.

1026. The TBM of embodiment 1018, in which the ABMs have a configuration selected from any one of the configurations designated Hv 71 through Hv 80.

1027. The TBM of embodiment 1018, in which the ABMs have a configuration selected from any one of the configurations designated Hv 81 through Hv 90.

1028. The TBM of embodiment 1018, in which the ABMs have a configuration selected from any one of the configurations designated Hv 91 through Hv 100.

1029. The TBM of embodiment 1018, in which the ABMs have a configuration selected from any one of the configurations designated Hv 101 through Hv 110.

1030. The TBM of embodiment 1018, in which the ABMs have a configuration selected from any one of the configurations designated Hv 111 through Hv 120.

1031. The TBM of embodiment 1018, in which the ABMs have a configuration selected from any one of the configurations designated Hv 121 through Hv 130.

1032. The TBM of embodiment 1018, in which the ABMs have a configuration selected from any one of the configurations designated Hv 131 through Hv 140.

1033. The TBM of embodiment 1018, in which the ABMs have a configuration selected from any one of the configurations designated Hv 141 through Hv 150.

1034. The TBM of embodiment 1018, in which the ABMs have a configuration selected from any one of the configurations designated Hv 151 through Hv 160.

1035. The TBM of embodiment 1018, in which the ABMs have a configuration selected from any one of the configurations designated Hv 161 through Hv 70.

1036. The TBM of embodiment 1018, in which the ABMs have a configuration selected from any one of the configurations designated Hv 171 through Hv 80.

1037. The TBM of embodiment 1018, in which the ABMs have a configuration selected from any one of the configurations designated Hv 181 through Hv 90.

1038. The TBM of embodiment 1018, in which the ABMs have a configuration selected from any one of the configurations designated Hv 191 through Hv 200.

1039. The TBM of embodiment 1018, in which the ABMs have a configuration selected from any one of the configurations designated Hv 201 through Hv 210.

1040. The TBM of embodiment 1018, in which the ABMs have a configuration selected from any one of the configurations designated Hv 211 through Hv 220.

1041. The TBM of embodiment 1018, in which the ABMs have a configuration selected from any one of the configurations designated Hv 221 through Hv 230.

1042. The TBM of embodiment 1018, in which the ABMs have a configuration selected from any one of the configurations designated Hv 231 through Hv 240.

1043. The TBM of any one of embodiments 1 to 453 and 575 to 1042, wherein any one, any two, or all three of ABM1, ABM2 and ABM3 has cross-species reactivity.

1044. The TBM of embodiment 1043, wherein ABM1 further binds specifically to CD2 in one or more non-human mammalian species.

1045. The TBM of embodiment 1043 or embodiment 1044, wherein ABM2 further binds specifically to the component of the TCR complex in one or more non-human mammalian species.

1046. The TBM of any one of embodiments 1043 to 1045, wherein ABM3 further binds specifically to the TAA in one or more non-human mammalian species.

1047. The TBM of any one of embodiments 1043 to 1046, wherein the one or more non-human mammalian species comprises one or more non-human primate species.

1048. The TBM of embodiment 1047, wherein the one or more non-human primate species comprises Macaca fascicularis.

1049. The TBM of embodiment 1047, wherein the one or more non-human primate species comprises Macaca mulatta.

1050. The TBM of embodiment 1047, wherein the one or more non-human primate species comprises Macaca nemestrina.

1051. The TBM of any one of embodiments 1043 to 1046, wherein the one or more non-human mammalian species comprises Mus musculus.

1052. The TBM of any one of embodiments 1 to 468 and 575 to 1051, wherein any one, any two, or all three of ABM1, ABM2 and ABM3 does not have cross-species reactivity.

1053. The TBM of any one of embodiments 575 to 1052, which has been recombinantly produced, optionally in a mammalian host cell, which is optionally selected from Vero cells, HeLa cells, COS cells, CHO cells, HEK293 cells, BHK cells and MDCKII cells.

1054. A conjugate comprising the TBM of any one of embodiments 1 to 468 or 575 to 1053 and an agent, optionally a therapeutic agent, a diagnostic agent, a masking moiety, a cleavable moiety, or any combination thereof.

1055. The conjugate of embodiment 1054, wherein the agent is a cytotoxic or cytostatic agent.

1056. The conjugate of embodiment 1055, wherein the agent is any of the agents described in Section 4.9.

1057. The conjugate of embodiment 1055 or 1056, wherein the agent is any of the agents described in Section 4.9.1.

1058. The conjugate of any one of embodiments 1054 to 1057, wherein the TBM is conjugated to a radionuclide.

1059. The conjugate of any one of embodiments 1054 to 1057, wherein the TBM is conjugated to an alkylating agent.

1060. The conjugate of any one of embodiments 1054 to 1057, wherein the TBM is conjugated to a topoisomerase inhibitor, which is optionally a topoisomerase I inhibitor or a topoisomerase II inhibitor.

1061. The conjugate of any one of embodiments 1054 to 1057, wherein the TBM is conjugated to a DNA damaging agent.

1062. The conjugate of any one of embodiments 1054 to 1057, wherein the TBM is conjugated to a DNA intercalating agent, optionally a groove binding agent such as a minor groove binding agent.

1063. The conjugate of any one of embodiments 1054 to 1057, wherein the TBM is conjugated to a RNA/DNA antimetabolite.

1064. The conjugate of any one of embodiments 1054 to 1057, wherein the TBM is conjugated to a kinase inhibitor.

1065. The conjugate of any one of embodiments 1054 to 1057, wherein the TBM is conjugated to a protein synthesis inhibitor.

1066. The conjugate of any one of embodiments 1054 to 1057, wherein the TBM is conjugated to a histone deacetylase (HDAC) inhibitor.

1067. The conjugate of any one of embodiments 1054 to 1057, wherein the TBM is conjugated to a mitochondrial. Inhibitor, which is optionally an inhibitor of a phosphoryl transfer reaction in mitochondria.

1068. The conjugate of any one of embodiments 1054 to 1057, wherein the TBM is conjugated to an antimitotic agent.

1069. The conjugate of any one of embodiments 1054 to 1057, wherein the TBM is conjugated to a maytansinoid.

1070. The conjugate of any one of embodiments 1054 to 1057, wherein the TBM is conjugated to a kinesin inhibitor.

1071. The conjugate of any one of embodiments 1054 to 1057, wherein the TBM is conjugated to a kinesin-like protein KIF11 inhibitor.

1072. The conjugate of any one of embodiments 1054 to 1057, wherein the TBM is conjugated to a V-ATPase (vacuolar-type H+-ATPase) inhibitor.

1073. The conjugate of any one of embodiments 1054 to 1057, wherein the TBM is conjugated to a pro-apoptotic agent.

1074. The conjugate of any one of embodiments 1054 to 1057, wherein the TBM is conjugated to a Bcl2 (B-cell lymphoma 2) inhibitor.

1075. The conjugate of any one of embodiments 1054 to 1057, wherein the TBM is conjugated to an MCL1 (myeloid cell leukemia 1) inhibitor.

1076. The conjugate of any one of embodiments 1054 to 1057, wherein the TBM is conjugated to a HSP90 (heat shock protein 90) inhibitor.

1077. The conjugate of any one of embodiments 1054 to 1057, wherein the TBM is conjugated to an IAP (inhibitor of apoptosis) inhibitor.

1078. The conjugate of any one of embodiments 1054 to 1057, wherein the TBM is conjugated to an mTOR (mechanistic target of rapamycin) inhibitor.

1079. The conjugate of any one of embodiments 1054 to 1057, wherein the TBM is conjugated to a microtubule stabilizer.

1080. The conjugate of any one of embodiments 1054 to 1057, wherein the TBM is conjugated to a microtubule destabilizer.

1081. The conjugate of any one of embodiments 1054 to 1057, wherein the TBM is conjugated to an auristatin.

1082. The conjugate of any one of embodiments 1054 to 1057, wherein the TBM is conjugated to a dolastatin.

1083. The conjugate of any one of embodiments 1054 to 1057, wherein the TBM is conjugated to a MetAP (methionine aminopeptidase).

1084. The conjugate of any one of embodiments 1054 to 1057, wherein the TBM is conjugated to a CRM1 (chromosomal maintenance 1) inhibitor.

1085. The conjugate of any one of embodiments 1054 to 1057, wherein the TBM is conjugated to a DPPIV (dipeptidyl peptidase IV) inhibitor.

1086. The conjugate of any one of embodiments 1054 to 1057, wherein the TBM is conjugated to a proteasome inhibitor.

1087. The conjugate of any one of embodiments 1054 to 1057, wherein the TBM is conjugated to a protein synthesis inhibitor.

1088. The conjugate of any one of embodiments 1054 to 1057, wherein the TBM is conjugated to a CDK2 (cyclin-dependent kinase 2) inhibitor.

1089. The conjugate of any one of embodiments 1054 to 1057, wherein the TBM is conjugated to a CDK9 (cyclin-dependent kinase 9) inhibitor.

1090. The conjugate of any one of embodiments 1054 to 1057, wherein the TBM is conjugated to a RNA polymerase inhibitor.

1091. The conjugate of any one of embodiments 1054 to 1057, wherein the TBM is conjugated to a DHFR (dihydrofolate reductase) inhibitor.

1092. The conjugate of any one of embodiments 1054 to 1057, wherein the agent is attached to the TBM with a linker, which is optionally a cleavable linker or a non-cleavabe linker, e.g.

1093. The conjugate of any one of embodiments 1054 to 1092, wherein the cytotoxic or cytostatic agent is conjugated to the TBM via a linker as described in Section 4.9.2.

1094. A preparation of TBMs comprising a plurality of TBM molecules according to any one of embodiments 575 to 1053 or a plurality of conjugate molecules according to any one of embodiments 1054 to 1093, optionally wherein the plurality comprises at least 100, at least 1,000, at least 10,000, or at least 100,000 TBM molecules or conjugate molecules.

1095. The preparation of embodiment 1094, wherein at least 50% of the trispecific molecules in the preparation have the same primary amino acid sequence.

1096. The preparation of embodiment 1094, wherein at least 60% of the trispecific molecules in the preparation have the same primary amino acid sequence.

1097. The preparation of embodiment 1094, wherein at least 70% of the trispecific molecules in the preparation have the same primary amino acid sequence.

1098. The preparation of embodiment 1094, wherein at least 80% of the trispecific molecules in the preparation have the same primary amino acid sequence.

1099. The preparation of embodiment 1094, wherein at least 90% of the trispecific molecules in the preparation have the same primary amino acid sequence.

1100. The preparation of embodiment 1094, wherein at least 95% of the trispecific molecules in the preparation have the same primary amino acid sequence.

1101. The preparation of embodiment 1094, wherein at least 97% of the trispecific molecules in the preparation have the same primary amino acid sequence.

1102. The preparation of embodiment 1094, wherein at least 98% of the trispecific molecules in the preparation have the same primary amino acid sequence.

1103. The preparation of embodiment 1094, wherein at least 99% of the trispecific molecules in the preparation have the same primary amino acid sequence.

1104. The preparation of embodiment 1094, wherein 50% to 95% of the trispecific molecules in the preparation have the same primary amino acid sequence.

1105. The preparation of embodiment 1094, wherein 50% to 80% of the trispecific molecules in the preparation have the same primary amino acid sequence.

1106. The preparation of embodiment 1094, wherein 50% to 70% of the trispecific molecules in the preparation have the same primary amino acid sequence.

1107. The preparation of embodiment 1094, wherein 60% to 95% of the trispecific molecules in the preparation have the same primary amino acid sequence.

1108. The preparation of embodiment 1094, wherein 60% to 80% of the trispecific molecules in the preparation have the same primary amino acid sequence.

1109. The preparation of embodiment 1094, wherein 60% to 70% of the trispecific molecules in the preparation have the same primary amino acid sequence.

1110. The preparation of embodiment 1094, wherein 70% to 95% of the trispecific molecules in the preparation have the same primary amino acid sequence.

1111. The preparation of embodiment 1094, wherein 70% to 80% of the trispecific molecules in the preparation have the same primary amino acid sequence.

1112. The preparation of embodiment 1094, wherein 80% to 95% of the trispecific molecules in the preparation have the same primary amino acid sequence.

1113. The preparation of embodiment 1094, wherein 95% to 99% of the trispecific molecules in the preparation have the same primary amino acid sequence.

1114. The preparation of any one of embodiments 1094 to 1113, wherein at least 50% of the trispecific molecules in the preparation have the same interchain crosslinks.

1115. The preparation of any one of embodiments 1094 to 1113, wherein at least 60% of the trispecific molecules in the preparation have the same interchain crosslinks.

1116. The preparation of any one of embodiments 1094 to 1113, wherein at least 70% of the trispecific molecules in the preparation have the same interchain crosslinks.

1117. The preparation of any one of embodiments 1094 to 1113, wherein at least 80% of the trispecific molecules in the preparation have the same interchain crosslinks.

1118. The preparation of any one of embodiments 1094 to 1113, wherein at least 90% of the trispecific molecules in the preparation have the same interchain crosslinks.

1119. The preparation of any one of embodiments 1094 to 1113, wherein at least 95% of the trispecific molecules in the preparation have the same interchain crosslinks.

1120. The preparation of any one of embodiments 1094 to 1113, wherein at least 97% of the trispecific molecules in the preparation have the same interchain crosslinks.

1121. The preparation of any one of embodiments 1094 to 1113, wherein at least 98% of the trispecific molecules in the preparation have the same interchain crosslinks.

1122. The preparation of any one of embodiments 1094 to 1113, wherein at least 99% of the trispecific molecules in the preparation have the same interchain crosslinks.

1123. The preparation of any one of embodiments 1094 to 1113, wherein 50% to 95% of the trispecific molecules in the preparation have the same interchain crosslinks.

1124. The preparation of any one of embodiments 1094 to 1113, wherein 50% to 80% of the trispecific molecules in the preparation have the same interchain crosslinks.

1125. The preparation of any one of embodiments 1094 to 1113, wherein 50% to 70% of the trispecific molecules in the preparation have the same interchain crosslinks.

1126. The preparation of any one of embodiments 1094 to 1113, wherein 60% to 95% of the trispecific molecules in the preparation have the same interchain crosslinks.

1127. The preparation of any one of embodiments 1094 to 1113, wherein 60% to 80% of the trispecific molecules in the preparation have the same interchain crosslinks.

1128. The preparation of any one of embodiments 1094 to 1113, wherein 60% to 70% of the trispecific molecules in the preparation have the same interchain crosslinks.

1129. The preparation of any one of embodiments 1094 to 1113, wherein 70% to 95% of the trispecific molecules in the preparation have the same interchain crosslinks.

1130. The preparation of any one of embodiments 1094 to 1113, wherein 70% to 80% of the trispecific molecules in the preparation have the same interchain crosslinks.

1131. The preparation of any one of embodiments 1094 to 1113, wherein 80% to 95% of the trispecific molecules in the preparation have the same interchain crosslinks.

1132. The preparation of any one of embodiments 1094 to 1113, wherein 95% to 99% of the trispecific molecules in the preparation have the same interchain crosslinks.

1133. The preparation of any one of embodiments 1094 to 1132, wherein at least 50% of the trispecific molecules in the preparation have the same ABM1:ABM2:ABM3 ratio.

1134. The preparation of any one of embodiments 1094 to 1132, wherein at least 60% of the trispecific molecules in the preparation have the same ABM1:ABM2:ABM3 ratio.

1135. The preparation of any one of embodiments 1094 to 1132, wherein at least 70% of the trispecific molecules in the preparation have the same ABM1:ABM2:ABM3 ratio.

1136. The preparation of any one of embodiments 1094 to 1132, wherein at least 80% of the trispecific molecules in the preparation have the same ABM1:ABM2:ABM3 ratio.

1137. The preparation of any one of embodiments 1094 to 1132, wherein at least 90% of the trispecific molecules in the preparation have the same ABM1:ABM2:ABM3 ratio.

1138. The preparation of any one of embodiments 1094 to 1132, wherein at least 95% of the trispecific molecules in the preparation have the same ABM1:ABM2:ABM3 ratio.

1139. The preparation of any one of embodiments 1094 to 1132, wherein at least 97% of the trispecific molecules in the preparation have the same ABM1:ABM2:ABM3 ratio.

1140. The preparation of any one of embodiments 1094 to 1132, wherein at least 98% of the trispecific molecules in the preparation have the same ABM1:ABM2:ABM3 ratio.

1141. The preparation of any one of embodiments 1094 to 1132, wherein at least 99% of the trispecific molecules in the preparation have the same ABM1:ABM2:ABM3 ratio.

1142. The preparation of any one of embodiments 1094 to 1132, wherein 50% to 95% of the trispecific molecules in the preparation have the same ABM1:ABM2:ABM3 ratio.

1143. The preparation of any one of embodiments 1094 to 1132, wherein 50% to 80% of the trispecific molecules in the preparation have the same ABM1:ABM2:ABM3 ratio.

1144. The preparation of any one of embodiments 1094 to 1132, wherein 50% to 70% of the trispecific molecules in the preparation have the same ABM1:ABM2:ABM3 ratio.

1145. The preparation of any one of embodiments 1094 to 1132, wherein 60% to 95% of the trispecific molecules in the preparation have the same ABM1:ABM2:ABM3 ratio.

1146. The preparation of any one of embodiments 1094 to 1132, wherein 60% to 80% of the trispecific molecules in the preparation have the same ABM1:ABM2:ABM3 ratio.

1147. The preparation of any one of embodiments 1094 to 1132, wherein 60% to 70% of the trispecific molecules in the preparation have the same ABM1:ABM2:ABM3 ratio.

1148. The preparation of any one of embodiments 1094 to 1132, wherein 70% to 95% of the trispecific molecules in the preparation have the same ABM1:ABM2:ABM3 ratio.

1149. The preparation of any one of embodiments 1094 to 1132, wherein 70% to 80% of the trispecific molecules in the preparation have the same ABM1:ABM2:ABM3 ratio.

1150. The preparation of any one of embodiments 1094 to 1132, wherein 80% to 95% of the trispecific molecules in the preparation have the same ABM1:ABM2:ABM3 ratio.

1151. The preparation of any one of embodiments 1094 to 1132, wherein 95% to 99% of the trispecific molecules in the preparation have the same ABM1:ABM2:ABM3 ratio.

1152. A pharmaceutical composition comprising the TBM of any one of embodiments 1 to 468 or 575 to 1053, the conjugate of any one of embodiments 1054 to 1093, or the preparation of any one of embodiments 1094 to 1151, and an excipient.

1153. A method of treating a subject with cancer, comprising administering to a subject suffering from cancer an effective amount of the TBM of any one of embodiments 1 to 468 or 575 to 1053, the conjugate of any one of embodiments 1054 to 1093, the preparation of any one of embodiments 1094 to 1151, or the pharmaceutical composition of embodiment 1152.

1154. The method of embodiment 1153, wherein the cancer is selected from HER2+ cancer, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

1155. The method of any of embodiment 1153 or embodiment 1154, wherein the TAA and indication are any of the TAA-indication combinations set forth in Table 14.

1156. The method of any one of embodiments 1153 to 1155, further comprising administering at least one additional agent to the subject.

1157. The method of embodiment 1156, wherein the additional agent is a chemotherapeutic agent.

1158. The method of embodiment 1156 or embodiment 1157, wherein the additional agent is an anthracycline.

1159. The method of embodiment 1156 or embodiment 1157, wherein the additional agent is a vinca alkaloid.

1160. The method of embodiment 1156 or embodiment 1157, wherein the additional agent is an alkylating agent.

1161. The method of embodiment 1156 or embodiment 1157, wherein the additional agent is an immune cell antibody.

1162. The method of embodiment 1156 or embodiment 1157, wherein the additional agent is an antimetabolite.

1163. The method of embodiment 1156 or embodiment 1157, wherein the additional agent is an adenosine deaminase inhibitor

1164. The method of embodiment 1156 or embodiment 1157, wherein the additional agent is an mTOR inhibitor.

1165. The method of embodiment 1156 or embodiment 1157, wherein the additional agent is a TNFR glucocorticoid induced TNFR related protein (GITR) agonist.

1166. The method of embodiment 1156 or embodiment 1157, wherein the additional agent is a proteasome inhibitor.

1167. The method of embodiment 1156 or embodiment 1157, wherein the additional agent is an immunomodulatory.

1168. The method of embodiment 1156 or embodiment 1157, wherein the additional agent is a thalidomide derivative.

1169. A nucleic acid or plurality of nucleic acids encoding the TBM of any one of embodiments 1 to 468 and 575 to 1053.

1170. The nucleic acid or plurality of nucleic acids of embodiment 1169 which is a DNA (are DNAs).

1171. The nucleic acid or plurality of nucleic acids of embodiment 1170 which are in the form of one or more vectors, optionally expression vectors.

1172. The nucleic acid or plurality of nucleic acids of embodiment 1169 which is a mRNA (are mRNAs).

1173. A cell engineered to express the TBM of any one of embodiments 1 to 468 and 575 to 1053.

1174. A cell transfected with one or more expression vectors comprising one or more nucleic acid sequences encoding the TBM of any one of embodiments 1 to 468 and 575 to 1053 under the control of one or more promoters.

1175. The cell of embodiment 1173 or embodiment 1174, wherein expression of the TBM is under the control of one or more inducible promoters.

1176. The cell of any one of embodiments 1173 to 1175, wherein the TBM is produced in secretable form.

1177. A method of producing a TBM, comprising:
(a) culturing the cell of any one of embodiments 1173 to 1177 in conditions under which the TBM is expressed; and
(b) recovering the TBM from the cell culture.

1178. An anti-CD3 antibody or antigen-binding fragment thereof, comprising the CDR sequences of CD3-21.

1179. The anti-CD3 antibody or antigen-binding fragment thereof of embodiment 1178, wherein the CDRs are defined by Kabat numbering, as set forth in Table 7B.

1180. The anti-CD3 antibody or antigen-binding fragment thereof of embodiment 1178, wherein the CDRs are defined by Chothia numbering, as set forth in Table 7C.

1181. The anti-CD3 antibody or antigen-binding fragment thereof of embodiment 1178, which comprises the heavy and light chain variable sequences of CD3-21 as set forth in Table 7A.

1182. The anti-CD3 antibody or antigen-binding fragment thereof of any one of embodiments 1178 to 1181, which is in the form of an antibody.

1183. The anti-CD3 antibody or antigen-binding fragment thereof of any one of embodiment 1182, which is in the form of a monospecific antibody.

1184. The anti-CD3 antibody or antigen-binding fragment thereof of any one of embodiment 1182, which is in the form of a multispecific antibody.

1185. The anti-CD3 antibody or antigen-binding fragment thereof of any one of embodiment 1182, which is in the form of a trispecfic antibody.

1186. The anti-CD3 antibody or antigen-binding fragment thereof of any one of embodiments 1178 to 1181, which is in the form of an antibody fragment.

1187. The anti-CD3 antibody or antigen-binding fragment thereof of embodiment 1186, which is in the form of a scFV.

1188. The anti-CD3 antibody or antigen-binding fragment thereof of embodiment 1187, comprising the CD3-21 scFv sequence set forth in Table 7A.

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A trispecific binding molecule (TBM), comprising:
(a) an antigen-binding module 1 (ABM1) that binds specifically to human CD2;
(b) an antigen-binding module 2 (ABM2) that binds specifically to a component of a human T-cell receptor (TCR) complex; and
(c) an antigen-binding module 3 (ABM3) that binds specifically to a human tumor-associated antigen (TAA),

2. The TBM of claim 1, wherein each antigen-binding module is capable of binding its respective target at the same time as each of the other antigen-binding modules is bound to its respective target.

3. The TBM of claim 1, wherein ABM1 is:
(a) an immunoglobulin scaffold-based ABM which is optionally an anti-CD2 antibody, an antibody fragment, an scFv, a dsFv, a Fv, a Fab, an scFab, a (Fab′)2, a single domain antibody (SDAB), a VH or VL domain, or a camelid VHH domain; or
(b) a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.

4. The TBM of claim 3, wherein ABM1 is an scFv or a Fab.

5. The TBM of claim 3, wherein ABM1 comprises any of the binding sequences set forth in Table 9.

6. The TBM of claim 1, wherein ABM1 comprises a receptor binding domain of a CD2 ligand.

7. The TBM of claim 1, wherein ABM1 is a CD58 moiety.

8. The TBM of claim 7, wherein ABM1 comprises amino acids 1-94 of CD58-2.

9. The TBM of claim 1, wherein ABM1 is a CD48 moiety.

10. The TBM of claim 1, wherein the component of a human TCR complex is CD3.

11. The TBM of claim 10, wherein ABM2 is:
(a) an immunoglobulin scaffold-based ABM which is optionally an anti-CD3 antibody, an antibody fragment, an scFv, a dsFv, a Fv, a Fab, an scFab, a (Fab′)2, a single domain antibody (SDAB), a VH or VL domain, or a camelid VHH domain; or
(b) a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.

12. The TBM of claim 11, wherein ABM2 is an scFv or Fab.

13. The TBM of claim 11, wherein ABM2 comprises any of the binding sequences set forth in any one of Tables 7A through 7D.

14. The TBM of claim 13, wherein ABM2 comprises the VH and VL sequences of CD3-21 as set forth in Table 7A.

15. The TBM of claim 1, wherein the component of a human TCR complex is the alpha subunit of the TCR.

16. The TBM of claim 15, wherein ABM2 is an anti-CD3 antibody, an antibody fragment, an scFv, a Fv, a dsFv, a Fab, an scFab, a (Fab′)2, a single domain antibody (SDAB), a VH or VL domain, a camelid VHH domain, a DARPins, an Avimer, an Anticalin/Lipocalin, a Centyrin, a Versabody, a Duocalin or a Fynomer.

17. The TBM of claim 1, wherein if TAA is a receptor, ABM3 comprises a receptor binding domain of a ligand of the receptor, and if TAA is a ligand, ABM3 comprises a ligand binding domain of a receptor of the ligand.

18. The TBM of claim 1, wherein ABM3 is:
(a) an immunoglobulin scaffold-based ABM which is optionally an anti-TAA antibody, an antibody fragment, an scFv, a dsFv, a Fv, a Fab, an scFab, a (Fab′)2, a single domain antibody (SDAB), a VH or VL domain, or a camelid VHH domain; or
(b) a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.

19. The TBM of claim 18, wherein the TAA is TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, EGFRvIII, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, FLT3, TAAG72, CD22, CD33, GD2, BCMA, gp100Tn, FAP, tyrosinase, EPCAM, CEA, Igf-I receptor, Cadherin17, CD32b, GPNMB, GPR64, HER3, LRP6, LYPD8, NKG2D, SLC34A2, SLC39A6, SLITRK6, TACSTD2, or EphB2.

20. The TBM of claim 18, wherein the TAA is BCMA.

21. The TBM of claim 20, wherein ABM3 comprises any of the binding sequences set forth in any one of Tables 12A, 12B, 12C, 12D, 12E, or 12F.

22. The TBM of claim 18, wherein the TAA is CD19.

23. The TBM of claim 22, wherein ABM3 comprises any of the binding sequences set forth Table 13.

24. The TBM of claim 18, wherein the TAA is Her2.

25. The TBM of claim 18, wherein the TAA is mesothelin.

26. The TBM of claim 18, wherein ABM3 is an scFv or a Fab.

27. The TBM of claim 1, which is a trivalent TBM.

28. The TBM of claim 27, wherein the trivalent TBM has any one of the configurations depicted in FIGS. 1B-1U and 1V-1Z.

29. The TBM of claim 28, which has the configuration depicted in FIG. 1I.

30. The TBM of claim 29, in which the ABMs have the configuration designated as T6.

31. The TBM of claim 1, which is a tetravalent TBM.

32. The TBM of claim 1, which is a pentavalent TBM.

33. The TBM of claim 1, which is a hexavalent TBM.

34. A conjugate comprising the TBM of any one of claims 1 to 33 and a cytotoxic or cytostatic agent.

35. A pharmaceutical composition comprising the TBM of any one of claims 1 to 33 and an excipient.

36. A method of treating a subject with cancer, comprising administering to a subject suffering from cancer an effective amount of the TBM of any one of claims 1 to 33.

37. The method of claim 36, wherein the cancer is selected from HER2+ cancer, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

38. A nucleic acid or plurality of nucleic acids encoding the TBM of any one of claims 1 to 33.

39. A cell engineered to express the TBM of any one of claims 1 to 33.

40. A cell transfected with one or more expression vectors comprising one or more nucleic acid sequences encoding the TBM of any one of claims 1 to 33 under the control of one or more promoters.

41. A method of producing a TBM, comprising:
(a) culturing the cell of claim 40 in conditions under which the TBM is expressed; and
(b) recovering the TBM from the cell culture.
